(19) United States
(12) Patent Application Publication
(10) Pub. No.: US 2008/0200535 A1
(43) Pub. Date: Aug. 21, 2008

(54) Amine Compounds

(75) Inventors: Yutaka Ohmori, Tokyo (JP); Masakazu Komatsu, Tokyo (JP)

Correspondence Address: BIRCH STEWART KOLASCH & BIRCH, PO BOX 747, FALLS CHURCH, VA, 22040-0747, US

(73) Assignee: Asahi Kasei Pharma Corporation, Tokyo (JP)

(21) Appl. No.: 11/892,674

(22) Filed: Aug. 24, 2007

Related U.S. Application Data
(60) Provisional application No. 60/841,026, filed on Aug. 29, 2006.

(30) Foreign Application Priority Data
Aug. 25, 2006 (JP) 2006-229187

(51) Int. Cl. A61K 31/381; C07C 217/74; C07D 333/24; A61K 31/135

(52) U.S. Cl. 514/438; 564/389; 514/655; 549/77

(57) ABSTRACT

There is provided a compound exhibiting an activity of suppressing immune response with reduced adverse drug reactions, which compound is useful in the chemotherapy for preventing or treating, for example, a wide range of various autoimmune diseases including systemic erythematodes, chronic rheumatoid arthritis, Type I diabetes, inflammatory bowel disease, biliary cirrhosis, uveitis, multiple sclerosis or other disorders, or chronic inflammatory diseases, or cancers, lymphoma or leukemia, or resistance to organ or tissue transplantation or rejection against transplantation.

Novel amine compounds having an S1P1/Edg1 receptor agonist effect, possible stereoisomers or racemic bodies of the compounds, or pharmacologically acceptable salts, hydrates or solvates of the compound, the stereoisomers or the racemic bodies, or prodrugs of the compounds, the stereoisomers, the racemic bodies, the salts, the hydrates or the solvates, are provided.

### CROSS-REFERENCE TO RELATED APPLICATION

The present patent application is the non-provisional application of US Provisional Patent Application No. 60/841,026, the disclosure of which is incorporated by reference herein.

### TECHNICAL FIELD

The present invention relates to novel amine compounds which have an S1P1/Edg1 receptor agonist effect, and thus are useful as an active ingredient for a pharmaceutical product which shows an immunosuppressive activity by causing lymphocyte sequestration in a secondary lymph tissue, and production intermediates of the compounds.

### BACKGROUND ART

For treatment of rheumatoid arthritis or other autoimmune diseases, anti-inflammatory drugs such as steroids have been used in the inflammatory reactions caused by abnormal immune responses, but these drugs are directed to symptomatic treatment and are not the fundamental remedy. Meanwhile, development of a method for suppressing immune response is very important in suppressing rejection in organ and cell transplantation, as well as in treating and preventing various autoimmune diseases. In fact, immunosuppressants appear to be effective for a wide range of various autoimmune diseases including systemic erythematosus, chronic rheumatoid arthritis, Type I diabetes, inflammatory bowel disease, biliary cirrhosis, uveitis, multiple sclerosis, or other disorders (for example, Crohn's diseases, ulcerative colitis, bullous pemphigoid, sarcoidosis, psoriasis, autoimmune myositis, Wegener's granulomatosis, ichthyosis, Graves' ophthalmopathy, atopic dermatitis, asthma, etc.), or chronic inflammatory diseases.

The respective fundamental etiology for autoimmune diseases are believed to be different, but they are associated in common with the appearance of various autoantibodies and/or autoreactive lymphocytes. Such autoreactiveness is partly caused by the loss of control of homeostasis that a normal immune system functions. Similarly, after bone marrow transplantation or organ transplantation, transplant rejection occurs when lymphocytes of the host recognize the antigens of foreign tissues, thus causing both cellular response, including antibodies, cytokines and cytotoxic lymphocytes, and humoral response.

The process of autoimmune response or the process of rejection leads to the destruction of tissues caused by inflammatory cells and/or intermediary factors released by inflammatory cells. Anti-inflammatory agents such as NSAIDs have an effect of inhibiting the action and secretion of such intermediary factors, but the agents cannot ameliorate immunological basis of the diseases.

Cyclosporine A and tacrolimus are drugs used to suppress the rejection of graft organs. Cyclosporine A and tacrolimus exert their effect by inhibiting the in vivo immune responses that are actuated to reject foreign proteins of the graft. Cyclosporine A and tacrolimus are effective in delaying or suppressing the rejection of grafts, but are known to cause some undesirable adverse drug reactions, including nephrotoxicity, neurotoxicity and gastrointestinal disorders. Therefore, the current status has not seen the development of an immunosuppressant having none of these adverse drug reactions. Based on such background, attempts are being made to find a compound having low toxicity and excellent immunosuppressive effects.

An immunosuppressive compound FTY720 is a lymphocyte sequestrant with which clinical trial is being performed at present.

The agonistic action of FTY720 against sphingosine 1-phosphate receptor induces sequestration of lymphocytes (T-cells and B-cells) in the lymph nodes and Peyer's patch, without being accompanied by depletion of lymph. That is, a sphingosine 1-phosphate receptor agonist may serve as an immunomodulatory material which induces the decrease in lymphocytes derived from the redistribution of lymphocytes from a circulation system to a secondary lymphoid tissues, without inducing systemic immunosuppression. Such immunosuppression is desirable to treat autoimmune disorders, or to suppress the rejection after organ transplantation.

However, it is reported that bradycardia is observed after the administration of FTY720 (Non-Patent Document 1), and thus sufficient care should be taken for the use. Thus, there is a demand for a medicament exhibiting higher effects and higher safety.

Sphingosine 1-phosphate had been considered to be an intermediate metabolic product in the metabolism of sphingosine, but the material was reported to have a cellular proliferation promoting effect or a controlling effect on cell movement function. Thus, it is becoming apparent that the material is a new lipid mediator which exhibits a variety of physiological actions, such as apoptotic action, cytomorphology modulating action and vasoconstriction. Sphingosine 1-phosphate exerts its action through a plurality of G protein conjugate receptors that are present on the surface of cell membrane. At present, five subtypes of sphingosine 1-phosphate receptors have been identified (S1P1, s1P2, S1P3, S1P4 and S1P5; these are also known by the name of endothelial differentiation genes, Edg1, Edg5, Edg3, Edg6 and Edg8), which have wide cellular distribution and tissue distribution, and are well maintained in humans and rodents. Activation of S1P1 and S1P3 through ligand induction appears to promote angiogenesis, chemotaxis, and adhesion conjugation structure, whereas the agonist effect of S1P2 promotes neurite retraction and inhibits chemotaxis of cells. S1P4 is localized in the cells and tissues of the hematopoietic system, whereas expression of S1P5 occurs mainly in neuron receptors, with some expression being observed in lymphoid tissues.

Administration of sphingosine 1-phosphate to animals induces systemic sequestration of peripheral blood lymphocytes to secondary lymphoid organs, thus resulting in therapeutically effective immunosuppression. However, sphingosine 1-phosphate also possesses cardiovascular action and bronchoconstriction action, which restricts the substance's usefulness as a therapeutic agent. Intravenous administration of sphingosine 1-phosphate lowers the heartbeat rate in rats (Non-Patent Document 2). Undesirable effects of sphingosine 1-phosphate are believed to be attributable to the non-selective agonist activity against every SIP receptor.

Under such circumstances, development of a compound which is selective to the S1P receptor subtype is desired.

Furthermore, as the compound having the same effects as the compound of the present invention, the compounds described in Patent Documents 1 to 3 are known, but they all differ from the compound of the present invention in the structural features.

[Patent Document 1] Brochure of International Patent Application Publication No. WO 03/105771

[Patent Document 2] Brochure of International Patent Application Publication No. WO 05/058848

[Patent Document 3] Brochure of International Patent Application Publication No. WO 02/044780

[Non-Patent Document 1] J. Am. Soc. Nephrol., 13, 1073 (2002)

[Non-Patent Document 2] Jpn. J. Pharmacol., 82, 338 (2000)

### DISCLOSURE OF THE INVENTION

### Problems to be Solved by the Invention

The object of the present invention is to provide a novel compound which suppresses immune responses with reduced adverse drug reactions. More particularly, the object is to provide a novel compound which is an S1P1 receptor agonist, that is, a novel compound showing an immunosuppressive activity by inducing lymphocyte sequestration in secondary lymphoid tissues It is another object of the present invention to provide a pharmaceutical product containing the compound as an active ingredient. More particularly, the object is to provide a radically therapeutic and/or prophylactic agent for autoimmune diseases and the like.

### Means to Solve the Problems

In an attempt to solve the problems described above, the inventors of the present invention decided to conduct a search for an agonist having an S1P1/Edg1 receptor-selective agonist activity, in particular, a compound having a high agonist activity against S1P1/Edg1 receptors as compared with S1P3s/Edg3 receptors, and devotedly conducted a search for the selective S1P1 receptor agonist. As a result, the inventors found that amine compounds represented by the following respective formulas, which are novel compounds, have excellent selective S1P1 receptor agonist activity, and that this compound is useful as an immunosuppressant. The present invention was achieved on the basis of the finding.

Thus, the present invention relates to the following.

[A1]

A compound represented by the following general formula (1):

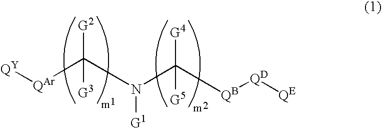

wherein

G¹ represents a hydrogen atom, or a C1-C4 alkyl group, with the proviso that the alkyl group may be substituted with one to three X^{G1} s, and when the alkyl group is substituted with two or more X^{G1}s, X^{G1}s may be the same or different;

X^{G1} represents a group selected from the group consisting of —OH, —CO₂H, —SO₃H, —PO₃H₂, and —OPO(OR^{G1})₂;

R^{G1} independently represents a hydrogen atom, or a C1-C4 alkyl group;

G² represents a hydrogen atom, a fluorine atom, a chlorine atom, or a C1-C4 alkyl group, with the proviso that the alkyl group may be substituted with one to three X^{G2}s, and when the alkyl group is substituted with two or more X^{G2}s, X^{G2}s may be the same or different;

X^{G2} represents a group selected from the group consisting of —OH, —CO₂H, —SO₃H, —PO₃H₂, and —OPO(OR^{G2})₂;

R^{G2} independently represents a hydrogen atom, or a C1-C4 alkyl group;

G³ represents a hydrogen atom, a fluorine atom, a chlorine atom, or a C1-C4 alkyl group, with the proviso that the alkyl group may be substituted with one to three X^{G3}s, and when the alkyl group is substituted with two or more X^{G3}s, X^{G3}s may be the same or different;

X^{G3} represents a group selected from the group consisting of —OH, —CO₂H, —SO₃H, —PO₃H₂, and —OPO(OR^{G3})₂;

R^{G3} independently represents a hydrogen atom, or a C1-C4 alkyl group;

G⁴ and G⁵, which may be the same or different, each independently represent a hydrogen atom, a fluorine atom, or a chlorine atom;

Q^{Ar} represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X^{QAr}s, and when these groups are substituted with two X^{QAr}s, X^{QAr}s may be the same or different;

X^{QAr} represents a fluorine atom, a chlorine atom, a bromine atom, a trifluoromethyl group, —OR^{XQAr}, —SR^{XQAr}, or —R^{XQAr};

R^{XQAr} represents a hydrogen atom, a C1-C6 alkyl group, a C3-C6 cycloalkyl group, or a phenyl group;

Q^{B} represents B^{Q1}, B^{Q2}, B³ or B⁴;

B^{Q1} represents a divalent group obtained by removing two hydrogen atoms from a compound selected from the group consisting of a 3- to 8-membered saturated ring compound composed of carbon atoms, a 3- to 8-membered partially unsaturated ring compound composed of carbon atoms, and a 3- to 8-membered unsaturated ring compound composed of carbon atoms, while these groups may be substituted to a possible extent with one to four X^{BQ1}s, and when these groups are substituted with two or more X^{BQ1}s, X^{BQ1}s may be the same or different;

X^{BQ1} represents a group selected from the group consisting of —OH, —CO₂H, —SO₃H, —PO₃H₂, —OPO(OR^{XBQ1)}₂, a 1H-tetrazol-5-yl group, a fluorine atom, a chlorine atom, an amino group, -G^{XB1}, —OG^{XB1} and —NG^{XB1}G^{XB1′}, or represents a group selected from the group consisting of the following formulas (B3-1) to (B3-5):

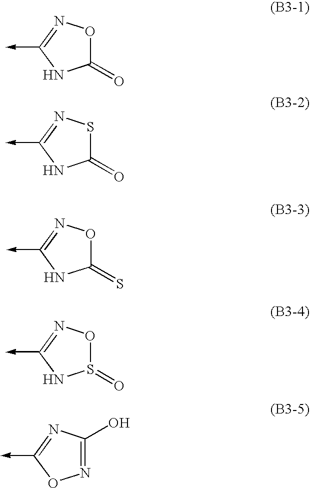

wherein the arrow represents the bonding position;

G^{XB1} and G^{XB1′}, which may be the same or different, each independently represent a C1-C4 alkyl group which may be substituted with 1 to 5 substituents selected from the group consisting of a halogen atom, a hydroxyl group, and an amino group;

R^{XBQ1} independently represents a hydrogen atom, or a C1-C4 alkyl group;

B^{Q2} represents a C1-C4 alkylene group, a C2-C4 alkenylene group, or a C2-C4 alkynylene group, while such group may be substituted to a possible extent with one to four X^{B2}s, and when these groups are substituted with two or more X^{B2}s, X^{B2}s may be the same or different;

X^{B2} represents a group selected from the group consisting of —OH, —CO₂H, —SO₃H, —PO₃H₂, —OPO(OR^{XBQ2})₂, a 1H-tetrazol-5-yl group, a fluorine atom, a chlorine atom, an amino group, -G^{XB2}, —OG^{XB2} and —NG^{XB2}G^{XB2′};

G^{XB2} and G^{XB2′}, which may be the same or different, each independently represent a C1-C4 alkyl group which may be substituted with 1 to 5 substituents selected from a halogen atom, a hydroxyl group, and an amino group;

R^{XBQ2} independently represents a hydrogen atom, or a C1-C4 alkyl group;

B³ represents a divalent group obtained by removing two hydrogen atoms from a compound that has a number of ring constituting atoms of 3 to 8, and that is obtained by substituting one to two carbon atoms in the ring of a compound selected from the group consisting of a saturated monocyclic hydrocarbon ring compound, a partially saturated monocyclic hydrocarbon ring compound, and a monocyclic aromatic hydrocarbon ring compound by an oxygen atom, a sulfur atom or a nitrogen atom, while these groups may be substituted to a possible extent with one to four X^{B3}s, and when these groups are substituted with two or more X^{B3}s, X^{B3}s may be the same or different;

X^{B3} represents a group selected from the group consisting of —OH, —CO₂H, —SO₃H, —PO₃H₂, —OPO(OR^{XBQ3)}₂, a 1H-tetrazol-5-yl group, a fluorine atom, a chlorine atom, an amino group, -G^{XB3}, —OG^{XB3} and —NG^{XB3}G^{XB3′};

R^{XBQ3} independently represents a hydrogen atom, or a C1-C4 alkyl group;

G^{XB3} and G^{XB3′}, which may be the same or different, each independently represent a C1-C4 alkyl group which may be substituted with 1 to 5 substituents selected from a halogen atom, a hydroxyl group, and an amino group;

B⁴ represents a divalent group obtained by removing two hydrogen atoms from a compound that has a number of ring constituting atoms of 7 to 11, and that is obtained by substituting 1 to 5 carbon atoms in the ring of a compound selected from the group consisting of a saturated bicyclic hydrocarbon ring compound, a partially saturated bicyclic hydrocarbon ring compound, and a bicyclic aromatic hydrocarbon ring compound by an oxygen atom, a sulfur atom or a nitrogen atom, while these groups may be substituted to a possible extent with one to four X^{B4}s, and when these groups are substituted with two or more X^{B4}s, X^{B4}s may be the same or different;

X^{B4} represents a group selected from the group consisting of —OH, —CO₂H, —CH₂CO₂H, —SO₃H, —PO₃H₂, —PO₂H₂, —OPO(OR^{XBQ4})₂, a 1H-tetrazol-5-yl group, a fluorine atom, a chlorine atom, an amino group, -G^{XB4}, —OG^{XB4} and —NG^{XB4}G^{XB4′};

R^{XBQ4} independently represents a hydrogen atom, or a C1-C4 alkyl group;

G^{XB4} and G^{XB4′}, which may be the same or different, each independently represent a C1-C4 alkyl group which may be substituted with 1 to 5 substituents selected from the group consisting of a halogen atom, a hydroxyl group, and an amino group;

Q^{D} represents a single bond, or a C1-C3 alkylene group which may be substituted with one to six fluorine atoms or chlorine atoms;

Q^{E} represents a group selected from the group consisting of —OH, CO₂R^{QE}, —CH₂CO₂R^{QE}, —SO₃H, —PO₃H₂, —PO₂H₂, —OPO(OR^{QE})₂ and a 1H-tetrazol-5-yl group, or represents a group selected from the group consisting of the following formulas (B3-1) to (B3-5):

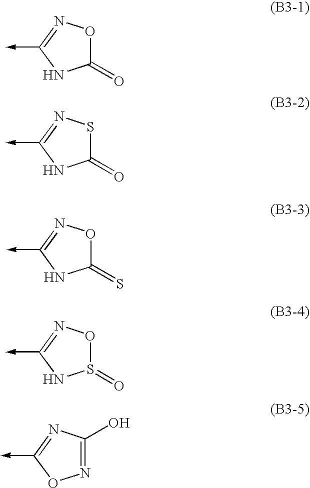

wherein the arrow represents the bonding position;

R^{QE} independently represents a hydrogen atom, a C1-C4 alkyl group, —(CH₂)_{mQ}N(R^{QE1})(R^{QE2}), or —C(R^{QE2)}₂OC(O)A^{QE}R^{QE4};

m^{Q} denotes an integer of 2 or 3;

R^{QE1} and R^{QE2}, which may be the same or different, each independently represent a methyl group, an ethyl group, or a propyl group, or R^{QE1} and R^{QE2} are joined to form a 3- to 6-membered ring together with the nitrogen atom, thus representing a saturated nitrogen-containing cycloalkyl group, or to form a morpholino group together with the nitrogen atom;

R^{QE3} independently represents a hydrogen atom, a methyl group, an ethyl group, or a propyl group;

R^{QE4} represents a C1-C4 alkyl group, a C3-C6 cycloalkyl group, or a phenyl group;

A^{QE} represents a single bond or an oxygen atom;

Q^{Y} represents Q^{W}-Q^{T}-Q^{Z}-(CG⁶G⁷)_{nQ}-Q^{V}-;

Q^{W} represents a hydrogen atom, a C1-C6 alkyl group which may be substituted with 1 to 7-fluorine atoms, a C3-C7 cycloalkyl group which may be substituted with 1 to 7 fluorine atoms, a C1-C6 alkoxy group which may be substituted with NR^{QW}R^{QW2}, a C3-C7 cycloalkoxy group which may be substituted with NR^{QW}R^{QW2}, a C1-C6 alkoxy group which may be substituted with a C3-C7 cycloalkyl group in which one to two carbon atoms in the ring may be substituted with a nitrogen atom(s), or a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X^{QW}s, and X^{QW}s may be the same or different, when the monovalent group is substituted with two X^{QW}s;

X^{QW} represents a fluorine atom, a chlorine atom, a trifluoromethyl group, a cyano group, —OR^{XQW}, —SR^{XQW}, or —R^{XQW};

R^{XQW} represents a hydrogen atom, a C1-C6 alkyl group, a C3-C6 cycloalkyl group, or a phenyl group;

R^{QW} and R^{QW2}, which may be the same or different, each independently represent a hydrogen atom, or a C1-C4 alkyl group;

Q^{T} represents a single bond, a C1-C6 alkylene group, —O—, —OCH₂—, —S—, —SO—, —SO₂—, —NR^{QT}—, —NR^{QT}NHCO—, or —CONR^{QT}—;

R^{QT} represents a hydrogen atom, or a C1-C4 alkyl group;

Q² is a single bond, or represents a C1-C6 alkylene group or a C3-C6 cycloalkylene group, or a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the Q^{Z} may be further substituted with one to four X^{QZ}s, and when Q^{Z} is substituted with two or more X^{QZ}s, X^{QZ}s may be the same or different;

X^{QZ} represents a fluorine atom, a chlorine atom, a trifluoromethyl group, a cyano group, —OR^{XQZ}, —SR^{XQZ}, or —R^{XQZ};

R^{XQZ} represents a hydrogen atom, a C1-C6 alkyl group, a C3-C6 cycloalkyl group, or a phenyl group, with the proviso that the C1-C6 alkyl group, the C3-C6 cycloalkyl group, and the phenyl group may be respectively substituted with a fluorine atom(s);

G⁶ and G⁷, which may be the same or different, each independently represent a hydrogen atom, or a C1-C4 alkyl group which may be substituted with 1 to 5 halogen atoms;

Q^{V} represents a single bond, —CO—, —COCR^{QV}—, —CR^{QV}R^{QV2}—, —S—, —SO—, —SO₂—, —SCH₂—, —SOCH₂—, —SO₂CH₂—, —O—, —CR^{QV}OR^{QV2}—, —CR^{QV}(OR^{QV2})CR^{QV3}R^{QV4}—, —CR^{QV}R^{QV2}CR^{QV3}(OR^{QV4})—, —C≡C—, —CR^{QV}CR^{QV2}, —NR^{QV}—, —NR^{QV}NHCO—, —CONR^{QV}—, or phenylene, or a divalent group obtained by removing two hydrogen atoms from oxadiazole or thiadiazole;

R^{QV}, R^{QV2}, R^{QV3}, and R^{QV4}, which may be the same or different, each independently represent a hydrogen atom, or a C1-C4 alkyl group;

n^{Q} denotes an integer of 0 to 2, with the proviso that when n^{Q} denotes 0, n^{Q} means a single bond;

m¹ denotes an integer of 1 to 3; and

m² denotes an integer of 0 to 3, with the proviso that when m² denotes 0, m² means a single bond,

or a possible stereoisomer or racemic body thereof, or a pharmacologically acceptable salt, hydrate or solvate of the compound, the stereoisomer or the racemic body, or a prodrug thereof.

[A2]

A compound represented by the following formula (2):

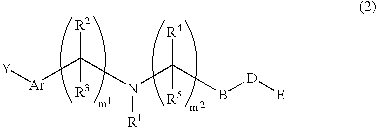

wherein

R¹, R², and R³, which may be the same or different, each independently represent a hydrogen atom, or a C1-C4 alkyl group;

R⁴ and R⁵, which may be the same or different, each independently, a hydrogen atom, a fluorine atom, or a chlorine atom;

Ar represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X¹s, and when these groups are substituted with two X¹s, X¹s may be the same or different;

X¹ represents a fluorine atom, a chlorine atom, a bromine atom, a trifluoromethyl group, —OR^{X1}, —SR^{X1}, or —R^{X1};

R^{X1} represents a hydrogen atom, a C1-C6 alkyl group, a C3-C6 cycloalkyl group, or a phenyl group;

B represents B¹ or B²;

B¹ represents a divalent group obtained by removing two hydrogen atoms from a compound selected from the group consisting of a 3- to 7-membered saturated ring compound composed of carbon atoms, a 3- to 7-membered partially unsaturated ring compound composed of carbon atoms, and a 3- to 7-membered unsaturated ring compound composed of carbon atoms, while these groups may be substituted to a possible extent with one to two X²s, and when these groups are substituted with two X²s, X²s may be the same or different;

X² represents a hydroxyl group, or a carboxyl group;

B² represents a C1-C4 alkylene group, a C2-C4 alkenylene group, or a C2-C4 alkynylene group, while such group may be substituted with one to two X³s, and when the group is substituted with two X³s, X³s may be the same or different;

X³ represents a fluorine atom, a carboxyl group, or a C1-C4 alkyl group which may be substituted with a hydroxyl group or a carboxyl group;

D represents a single bond, a methylene group or an ethylene group;

E represents a hydroxyl group, —CO₂R^{E}, or a 1H-tetrazol-5-yl group;

R^{E} represents a hydrogen atom, a C1-C4 alkyl group, —(CH₂)ₘN(R^{E1})(R^{E2}), or —C(R^{E3})₂OC(O)A^{E}R^{E4};

m denotes an integer of 2 or 3;

R^{E1} E and R^{E2}, which may be the same or different, each independently represents a methyl group, an ethyl group or a propyl group, or R^{E1} and R^{E2} are joined to form a 3- to 6-membered ring together with the nitrogen atom, thus representing a saturated nitrogen-containing cycloalkyl group, or to form a morpholino group together with the nitrogen atom;

R^{E3} represents a hydrogen atom, a methyl group, an ethyl group, or a propyl group;

R^{E4} represents a C1-C4 alkyl group, a C3-C6 cycloalkyl group, or a phenyl group;

A^{E} represents a single bond or an oxygen atom;

Y represents W-T-Z-(CR⁶R⁷)ₙ—V—;

W represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{W}R^{W2}, a C3-C7 cycloalkoxy group which may be substituted with NR^{W}R^{W2}, or a C1-C6 alkoxy group which may be substituted with a C3-C7 cycloalkyl group in which one to two carbon atoms in the ring may be substituted with a nitrogen atom(s), or a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X⁴s and when the monovalent group is substituted with two X⁴s, X⁴s may be the same or different;

X⁴ represents a fluorine atom, a chlorine atom, a trifluoromethyl group, a trifluoromethoxy group, a cyano group, —OR^{X4}, —SR^{X4}, or —R^{X4};

R^{X4} represents a hydrogen atom, a C1-C6 alkyl group, a C3-C6 cycloalkyl group, or a phenyl group;

R^{W} and R^{W2}, which may be the same or different, each independently represent a hydrogen atom, or a C1-C6 alkyl group;

T represents a single bond, a C1-C6 alkylene group, —O—, —OCH₂—, —S—, —SO—, —SO₂—, —NR^{T}—, —NR^{T}NHCO—, or —CONR^{T}—;

R^{T} represents a hydrogen atom, or a C1-C6 alkyl group;

Z represents a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the Z may be further substituted with one to four X⁵s, and when Z is substituted with two or more X⁵s, X⁵s may be the same or different;

X⁵ represents a fluorine atom, a chlorine atom, a trifluoromethyl group, a cyano group, —OR^{X5}, —SR^{X5}, or R^{X5};

R^{X5} represents a hydrogen atom, a C1-C6 alkyl group, a C3-C6 cycloalkyl group, or a phenyl group, with the proviso that the C1-C6 alkyl group, the C3-C6 cycloalkyl group and the phenyl group may be respectively substituted with a fluorine atom(s);

R⁶ and R⁷, which may be the same or different, each independently represent a hydrogen atom, or a C1-C4 alkyl group;

V represents a single bond, —CO—, —COCR^{V}—, —CR^{V}R^{V2}—, —S—, —SO—, —SO₂—, —SCH₂—, —SOCH₂—, —SO₂CH₂—, —O—, —CR^{V}OR^{V2}—, CR^{V}(OR^{V2})CR^{V3}R^{V4}—, —CR^{V}R^{V2}CR^{V3}(OR^{V4})—, —C≡C—, —CR^{V}═CR^{V2}, —NR^{V}—, —NR^{V}NHCO—, or —CONR^{V}—, or represents a divalent group obtained by removing two hydrogen atoms from oxadiazole or thiadiazole;

R^{V}, R^{V2}, R^{V3}, and, R^{V4}, which may be the same or different, each independently represent a hydrogen atom, or a C1-C4 alkyl group;

n denotes an integer of 0 to 2, with the proviso that when n denotes 0, n means a single bond;

m¹ denotes an integer of 1 to 3; and

m² denotes an integer of 0 to 3, with the proviso that when m² denotes 0, m 2 means a single bond,

or a possible stereoisomer or racemic body thereof, or a pharmacologically acceptable salt, hydrate or solvate of the compound, the stereoisomer or the racemic body, or a prodrug thereof.

[A3]

A compound represented by the following general formula (3):

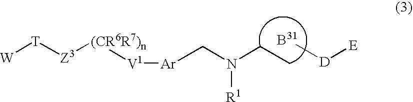

wherein W, T, R⁶, R⁷, n, Ar, R¹, D, and E have the same meanings as the defined above; B³¹ represents a divalent group obtained by removing two hydrogen atoms from a 3- to 7-membered saturated ring compound composed of carbon atoms; Z³ represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine, and imidazo[1,2-a]pyridine;

with the proviso that the Z³ may be further substituted with one to four X^{Z3}s, and when Z³ is substituted with two or more X^{Z3}s, X^{Z3}s may be the same or different;

X^{Z3} represents a fluorine atom, a chlorine atom, a trifluoromethyl group, a cyano group, —OR^{XZ3}, —SR^{XZ3}, or —R^{XZ3};

R^{XZ3} represents a hydrogen atom, a C1-C6 alkyl group, a C3-C6 cycloalkyl group, or a phenyl group, with the proviso that the C1-C6 alkyl group, the C3-C6 cycloalkyl group, the phenyl group may be respectively substituted with a fluorine atom(s);

V¹ represents a single bond, —CO—, —COCR^{V1}R^{V12}—, —CR^{V1}R^{V12}—, —O—, CR^{V1}OR^{V12}—, CR^{V1}(OR^{V12})CR^{V13}R^{V14}—, —CR^{V1}R^{V12}CR^{V13}(OR^{V14})—, C≡C—, —CR^{V1}═CR^{V12}—, —NR^{V1}—, —NR^{V1}NHCO—, or —CONR^{V1}—, or represents a divalent group obtained by removing two hydrogen atoms from oxadiazole or thiadiazole; and

R^{V1}, R^{V12}, R^{V13}, and R^{V14}, which may be the same or different, each independently represent a hydrogen atom, or a C1-C4 alkyl group,

or a possible stereoisomer or racemic body thereof, or a pharmacologically acceptable salt, hydrate or solvate of the compound, the stereoisomer or the racemic body, or a prodrug thereof.

[A4]

The compound according to [A3], wherein B³¹ represents a divalent group obtained by removing two hydrogen atoms from a 4-membered saturated ring compound composed of carbon atoms, and the positions at which B³¹ is bound to —NR¹— and -D-E are (1,3) of the 4-membered saturated ring compound, or a possible stereoisomer or racemic body thereof, or a pharmacologically acceptable salt, hydrate or solvate of the compound, the stereoisomer or the racemic body, or a prodrug thereof.

[A4-2]

The compound according to [A3], wherein B³¹ represents a divalent group obtained by removing two hydrogen atoms from a 4-membered saturated ring compound composed of carbon atoms, the positions at which B³¹ is bound to —NR¹— and -D-E are (1,3) of the 4-membered saturated ring compound, and the relationship between the bond between B³¹ and —NR¹—, and the bond between B³¹ and -D-E is a cis relationship,

or a possible stereoisomer or racemic body thereof, or a pharmacologically acceptable salt, hydrate or solvate of the compound, the stereoisomer or the racemic body, or a prodrug thereof.

[A4-3]

The compound according to [A3], wherein B³¹ represents a divalent group obtained by removing two hydrogen atoms from a 4-membered saturated ring compound composed of carbon atoms, the positions at which B³¹ is bound to —NR¹— and -D-E are (1,3) of the 4-membered saturated ring compound, and the relationship between the bond between B³¹ and —NR¹—, and the bond between B³¹ and -D-E is a trans relationship,

or a possible stereoisomer or racemic body thereof, or a pharmacologically acceptable salt, hydrate or solvate of the compound, the stereoisomer or the racemic body, or a prodrug thereof.

[A5]

The compound described in any one of [A3] to [A4-3], wherein D represents a single bond, E represents CO₂R^{E} (provided that R^{E} has the same meaning as the defined above),

or a possible stereoisomer or racemic body thereof, or a pharmacologically acceptable salt, hydrate or solvate of the compound, the stereoisomer or the racemic body, or a prodrug thereof.

When the item numbers to be cited are shown in a range, such as [A3] to [A4-3], and there are items having branch numbers within the range, such as [A4-2], then it means that the items having branch numbers such as [A4-2] are also cited. The same applies to the descriptions that follows.

[A6]

The compound described in any one of [A3] to [A5], wherein Ar represents a divalent group obtained by removing two hydrogen atoms from a compound selected from the group consisting of benzene and thiophene,

or a possible stereoisomer or racemic body thereof, or a pharmacologically acceptable salt, hydrate or solvate of the compound, the stereoisomer or the racemic body, or a prodrug thereof.

[A6-2]

The compound described in anyone of [A3] to [A5], wherein Ar represents a divalent group obtained by removing two hydrogen atoms from benzene,

or a possible stereoisomer or racemic body thereof, or a pharmacologically acceptable salt, hydrate or solvate of the compound, the stereoisomer or the racemic body, or a prodrug thereof.

[A7]

The compound described in any one of [A3] to [A6-2], wherein V¹ represents a divalent group obtained by removing two hydrogen atoms from oxadiazole or thiadiazole,

or a possible stereoisomer or racemic body thereof, or a pharmacologically acceptable salt, hydrate or solvate of the compound, the stereoisomer or the racemic body, or a prodrug thereof.

[A8]

The compound described in any one of [A3] to [A7], wherein W represents a monovalent group obtained by removing one hydrogen atom from a compound selected from the group consisting of benzene, furan and thiophene, each of which may be substituted with one or two groups each independently selected from the group consisting of a fluorine atom, a chlorine atom, a cyano group, and a trifluoromethyl group,

or a possible stereoisomer or racemic body thereof, or a pharmacologically acceptable salt, hydrate or solvate of the compound, the stereoisomer or the racemic body, or a prodrug thereof.

[A9]

The compound described in anyone of [A3] to [A7], wherein W represents a hydrogen atom, a C1-C6 alkyl group which may be substituted with 1 to 7 fluorine atoms, or a C3-C7 cycloalkyl group which may be substituted with 1 to 7 fluorine atoms,

or a possible stereoisomer or racemic body thereof, or a pharmacologically acceptable salt, hydrate or solvate of the compound, the stereoisomer or the racemic body, or a prodrug thereof.

[A10]

The compound described in anyone of [A3] to [A9], wherein Z³ represents a divalent group obtained by removing two hydrogen atoms from a compound selected from benzene and thiophene, each of which may be substituted with one to four groups each independently selected from the group consisting of a fluorine atom, a chlorine atom, a cyano group, a trifluoromethyl group and a methyl group,

or a possible stereoisomer or racemic body thereof, or a pharmacologically acceptable salt, hydrate or solvate of the compound, the stereoisomer or the racemic body, or a prodrug thereof.

[A11]

The compound according to [A3], wherein

R¹ represents a hydrogen atom;

Ar represents a divalent group obtained by removing two hydrogen atoms from benzene, while the divalent group may be substituted with one to two X¹s, and when the divalent group is substituted with two X¹s, X¹s may be the same or different, with X¹ representing a fluorine atom, a chlorine atom, a bromine atom, —OR^{X1}, or —R^{X1};

R^{X1} represents a hydrogen atom, a methyl group, an ethyl group, a propyl group, or an isopropyl group;

B³¹ represents a divalent group obtained by removing two hydrogen atoms from a 4- to 5-membered saturated ring compound composed of carbon atoms;

D represents a single bond;

E represents CO₂R^{E}; R^{E} represents a hydrogen atom; a methyl group, an ethyl group, —(CH₂)N(R^{E1})(R^{E2}), or —C(R^{E3})₂OC(O)A^{E}R^{E4}; m denotes an integer of 2 or 3; R^{E1} and R^{E2} may be the same or different, and each independently represent a methyl group, an ethyl group or a propyl group, or R^{E1} and R^{E2} are joined to form a 3- to 6-membered ring together with the nitrogen atom, thus representing a saturated nitrogen-containing cycloalkyl group, or to form a morpholino group together with the nitrogen atom; R^{E3} represents a hydrogen atom, a methyl group, an ethyl group, or a propyl group; R^{E4} represents a C1-C4 alkyl group, a C3-C6 cycloalkyl group, or a phenyl group; A^{E} represents a single bond or an oxygen atom;

W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene and pyridine, with the proviso that the monovalent group obtained by removing one hydrogen atom from a compound selected from the group consisting of benzene, naphthalene, furan, thiophene and pyridine, may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group, an ethyl group, a propyl group, an isopropyl group, a cyclopentyl group, a cyclohexyl group or a phenyl group;

T represents a single bond, a methylene group, an ethylene group, or —O—;

Z³ represents a C5-C6 cycloalkylene group, or a divalent group obtained by removing two hydrogen atoms from a compound selected from the group consisting of benzene, thiophene, and pyridine, while Z³ may be substituted with one to four X^{Z3}s, and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group, or a propyl group;

V¹ represents a divalent group obtained by removing two hydrogen atoms from oxadiazole or thiadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-2]

The compound according to [A3], wherein

R¹ represents a hydrogen atom;

Ar represents a divalent group obtained by removing two hydrogen atoms from benzene, while the divalent group may be substituted with one to two X¹s, and when the divalent group is substituted with two X¹s, X¹s may be the same or different, with X¹ representing —OR^{X1} or —R^{X1}, and R^{X1} representing a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1, 3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from a compound selected from the group consisting of benzene and thiophene, while the group may be substituted with one to two X⁴s, and when the group is substituted with two X⁴s, X⁴s may be the same or different, with X⁴ represents a fluorine atom, a trifluoromethyl group, a cyano group or —R^{X4}, and R^{X4} representing a methyl group or an ethyl group,

T represents a single bond or —O—,

Z³ represents a divalent group obtained by removing two hydrogen atoms from benzene or thiophene;

X^{Z3} represents a fluorine atom, a trifluoromethyl group, a cyano group, or —R^{XZ3};

R^{XZ3} represents a methyl group;

V¹ represents a divalent group obtained by removing two hydrogen atoms from [1,2,4]-oxadiazole; and

n is 0,

or a possible stereoisomer or racemic body thereof, or a pharmacologically acceptable salt, hydrate or solvate of the compound, the stereoisomer or the racemic body, or a prodrug thereof.

[A11-3]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom, a chlorine atom, a bromine atom, —OR^{X1} or —R^{X1};

R^{X1} represents a hydrogen atom, a methyl group or an ethyl group,

B³¹ represents a divalent group obtained by removing two hydrogen atoms from a 4-membered saturated ring compound composed of carbon atoms, while the positions at which B³¹ is bound to —NR¹— and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and pyridine, may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group, an ethyl group, a propyl group, an isopropyl group, a cyclopentyl group, a cyclohexyl group or a phenyl group;

T represents a single bond or —O—;

Z³ represents a divalent group obtained by removing two hydrogen atoms from a compound selected from the group consisting of benzene, thiophene and pyridine, while Z³ may be substituted with one to four X^{Z3}s, and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group, or a propyl group;

V¹ represents a divalent group obtained by removing two hydrogen atoms from oxadiazole or thiadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-4]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom, a chlorine atom, a bromine atom, —OR^{X1}, or —R^{X1};

R^{X1} represents a hydrogen atom, a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or R^{X4}, and R^{X4} representing a hydrogen atom, a methyl group, an ethyl group, a propyl group, an isopropyl group, a cyclopentyl group, a cyclohexyl group or a phenyl group;

T represents a single bond;

Z³ represents a divalent group obtained by removing two hydrogen atoms from benzene, while Z³ may be substituted with one to four X^{Z3}s , and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from oxadiazole or thiadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-5]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom, a chlorine atom, a bromine atom, —OR^{X1} or —R^{X1};

R^{X1} represents a hydrogen atom, a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and pyridine, may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when Z³ is substituted with two or more X^{Z3} s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-6]

The compound according to [A3], wherein

R¹ represents a hydrogen atom;

Ar represents a divalent group obtained by removing two hydrogen atoms from benzene, while the group may be substituted with one to two X¹s, and when the divalent group is substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and, pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-7]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹— and -D-E are (1,3) of the 4-membered saturated ring compound, and the relationship between the bond between B³¹ and —NR¹-, and the bond between B³¹ and -D-E is a trans relationship;

D represents a single bond;

E represents CO₂R^{E}, and R^{E} represents a hydrogen atom, a methyl group or an ethyl group;

W represents a C1-C6 alkyl group which may be substituted with 1 to 7 fluorine atoms, or a C3-C7 cycloalkyl group which may be substituted with 1 to 7 fluorine atoms;

T represents a single bond;

Z³ represents a divalent group obtained by removing two hydrogen atoms from benzene, while Z³ may be substituted with one to four X^{Z3}s, and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-8]

The compound according to [A3], wherein

R¹ represents a hydrogen atom;

Ar represents a divalent group obtained by removing two hydrogen atoms from benzene and thiophene, while these groups may be substituted with one to two X¹s, and when these groups are substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when Z³ is substituted with two or more X^{Z3}s, X^{Z5} may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-9]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1,3) of the 4-membered saturated ring compound, and the relationship between the bond between B3′ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from a compound selected from the group consisting of benzene, furan, thiophene and pyridine, with the proviso that the monovalent group obtained by removing one hydrogen atom from a compound selected from the group consisting of benzene, furan, thiophene and pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}, and when Z³ is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-10]

The compound according to [A3], wherein

R¹ represents a hydrogen atom;

Ar represents a divalent group obtained by removing two hydrogen atoms from benzene and thiophene, while these groups may be substituted with one to two X¹s, and when these groups are substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹— and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R⁹, and R^{E} represents a hydrogen atom, a methyl group or an ethyl group;

W represents a monovalent group obtained by removing one hydrogen atom from benzene, with the proviso that the monovalent obtained by removing one hydrogen atom from benzene may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when the divalent group is substituted with two or more X^{Z3}s, X^{Z3}s may be the same or different, with X^{Z3} representing a fluorine atom, a trifluoromethyl group, a cyano group, OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-11]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹— and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from benzene, with the proviso that the monovalent group obtained by removing one hydrogen atom from benzene may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when Z³ is substituted with two or more X^{Z3} s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-12]

The compound according to [A3], wherein

R¹ represents a hydrogen atom;

Ar represents a divalent group obtained by removing two hydrogen atoms from benzene or thiophene, while these groups may be substituted with one to two X¹s, and when these groups are substituted with two X¹s, X¹s may be the same or different, with X¹ representing a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹- and -D-E are (1, 3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from pyridine, with the proviso that the monovalent group obtained by removing on hydrogen atom from pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, OR^{X4} or —R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s and when the divalent group is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-13]

The compound according to [A3], wherein

R¹ represents a hydrogen atom;

Ar represents a divalent group obtained by removing two hydrogen atoms from benzene, while the group may be substituted with one to two X¹s, and when the group is substituted with two X¹s, X¹s may be the same or different, with X¹ represents a fluorine atom or —R^{X1};

R^{X1} represents a methyl group or an ethyl group;

B³¹ represents a divalent group obtained by removing two hydrogen atoms from a 4-membered saturated ring compound composed of carbon atoms, while the positions at which B³¹ is bound to —NR¹ and -D-E are (1,3) of the 4-membered saturated ring compound, and the relationship between the bond between B³¹ and —NR¹—, and the bond between B³¹ and -D-E is a trans relationship;

D represents a single bond;

E represents CO₂R^{E}, and R^{E} represents a hydrogen atom, a methyl group or an ethyl group;

W represents a monovalent group obtained by removing one hydrogen atom from pyridine, with the proviso that the monovalent group obtained by removing one hydrogen atom from pyridine may be substituted with one to two X⁴s, and when the monovalent group is substituted with two X⁴s, X⁴s may be the same or different, with X⁴ representing a fluorine atom, a trifluoromethyl group, a trifluoromethoxy group, a cyano group, —OR^{X4} or R^{X4}, and R^{X4} representing a hydrogen atom, a methyl group or an ethyl group;

T represents a single bond;

Z³ represents a divalent group obtained by removing two hydrogen atoms from benzene, while Z³ may be substituted with one to four X^{Z3}s, and when the divalent group is substituted with two or more X^{Z3}s, X^{Z3}s may be the same or different, with X^{Z3} representing a fluorine atom, a trifluoromethyl group, a cyano group, —OR^{XZ3} or —R^{XZ3}, and R^{XZ3} representing a methyl group, an ethyl group or a propyl group;

V¹ represents a divalent group obtained by removing two hydrogen atoms from [1,2,4]-oxadiazole; and

n denotes 0 or 1,

or a possible stereoisomer or racemic body thereof, or a pharmacologically acceptable salt, hydrate or solvate of the compound, the stereoisomer or the racemic body, or a prodrug thereof.

[A11-14]

The compound according to any one of [A11-2], or [A11-5] to [A11-13], wherein the binding positions of W-T-Z³-(CR⁶R⁷)ⁿ— and —Ar—CH₂—NR¹—B^{B31}-D-E with respect to the divalent group obtained by removing two hydrogen atoms from [1, 2,4]-oxadiazole are (5, 3),

or a possible stereoisomer or racemic body thereof, or a pharmacologically acceptable salt, hydrate or solvate of the compound, the stereoisomer or the racemic body, or a prodrug thereof.

[A12]

A compound represented by the following general formula (6):

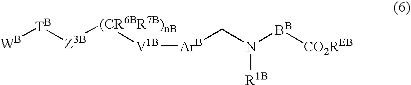

wherein

R^{1B} represents a hydrogen atom, or a C1-C4 alkyl group;

Ar^{B} represents a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine;

these groups may be substituted with one to two X^{1B}s, and when these groups are substituted with two X^{1B}s, X^{1B}s may be the same or different;

X¹⁸ represents a fluorine atom, a chlorine atom, a bromine atom, a trifluoromethyl group, —OR^{X1B}, —SR^{X1B}, or —R^{X1B};

R^{X1B} represents a hydrogen atom, a C1-C6 alkyl group, a C3-C6 cycloalkyl group, or a phenyl group;

B^{B} represents a C2 alkylene group, while the group may be substituted to a possible extent with one to four X^{BB}s, and when the group is substituted with two or more X^{BB}s, X^{BB}s may be the same or different;

X^{BB} represents a fluorine atom, or a C1-C4 alkyl group which may be substituted with one to five G^{XBB}s, and when the alkyl group is substituted with two or more G^{XBB}s, G^{XBB}s may be the same or different;

G^{XBB} represents a halogen atom, a hydroxyl group or an amino group;

R^{EB} represents a hydrogen atom, a C1-C4 alkyl group, —(CH₂)_{mB}N(R^{EB1})(R^{EB2}, or —C(R^{EB3})₂OC(O)A^{EB}R^{EB4};

m^{B} denotes an integer of 2 or 3;

R^{EB1} and REB², which may be the same or different, each independently represent a methyl group, an ethyl group or a propyl group, or R^{EB1} and R^{EB2} are joined to form a 3- to 6-membered ring together with the nitrogen atom, thus representing a saturated nitrogen-containing cycloalkyl group, or to form a morpholino group together with the nitrogen atom;

R^{EB3} represents a hydrogen atom, a methyl group, an ethyl group or a propyl group;

R^{EB4} represents a C1-C4 alkyl group, a C3-C6 cycloalkyl group or a phenyl group;

A^{EB} represents a single bond or an oxygen atom;

V^{1B} represents a single bond, or a divalent group obtained by removing two hydrogen atoms from a compound selected from the group consisting of oxadiazole and thiadiazole;

W^{B} represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{WB}R^{WB2}, a C3-C7 cycloalkoxy group which may be substituted with NR^{WB}R^{WB2}, or a C1-C6 alkoxy group which may be substituted with a C3-C7 cycloalkyl group in which one to two carbon atoms in the ring may be substituted with a nitrogen atom(s), or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring may be substituted with one to two X^{4B}s, and when the monovalent group is substituted with two X^{4B}s, X^{4B}s may be the same or different;

X^{4B} represents a fluorine atom, a chlorine atom, a trifluoromethyl group, a trifluoromethoxy group, a cyano group, —OR^{X4B}, —SR^{X4B}, or R^{X4B};

R^{X4B} represents a hydrogen atom, a C1-C6 alkyl group, a C3-C6 cycloalkyl group, or a phenyl group;

R^{WB} and R^{WB2}, which may be the same or different, each independently represent a hydrogen atom, or a C1-C6 alkyl group;

T^{B} represents a single bond, a C1-C6 alkylene group, —O—, —OCH₂—, —NR^{TB}—, —NR^{TB}NHCO—, or —CONR^{TB}—;

R^{TB} represents a hydrogen atom, or a C1-C6 alkyl group;

Z^{3B} represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalener benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine and imidazo[1,2-a]pyridine;

with the proviso that Z^{3B} may be further substituted with one to four X^{Z3B}s, and when Z^{3B} is substituted with two or more X^{Z3B}s, X^{Z3B}s may be same or different;

X^{Z3B} represents a fluorine atom, a chlorine atom, a trifluoromethyl group, a cyano group, —OR^{XZ3B}, —SR^{XZ3B}, or —R^{XZ3B};

R^{XZ3B} represents a hydrogen atom, a C1-C6 alkyl group, a C3-C6 cycloalkyl group or a phenyl group, provided that the C1-C6 alkyl group, the C3-C6 cycloalkyl group and the phenyl group may be respectively substituted with a fluorine atom(s);

when V^{1B} is a single bond, n^{B} denotes 0, and Z^{3B} is a single bond, or a divalent group obtained by removing two hydrogen atoms from a compound selected from the group consisting of furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine;

when V^{1B} is a single bond, and Z^{3B} is a single bond, T^{B} represents a single bond, and W^{B} represents a divalent group obtained by removing one hydrogen atom from a monocyclic aromatic heterocyclic compound;

R^{6B} and R^{7B}, which may be the same or different, each independently represent a hydrogen atom or a C1-C4 alkyl group; and

n^{B} denotes an integer of 0 to 2, with the proviso that when n^{B} denotes 0, it means a single bond,

or a possible stereoisomer or racemic body thereof, or a pharmacologically acceptable salt, hydrate or solvate of the compound, the stereoisomer or the racemic body, or a prodrug thereof.

[A13]

The compound according to [A12], wherein B^{B} represents a C2 alkylene group, while the group may be substituted with one to four C1-C4 alkyl groups, and when the group is substituted with two or more C1-C4 alkyl groups, the C1-C4 alkyl groups may be the same or different,

or a possible stereoisomer or racemic body thereof, or a pharmacologically acceptable salt, hydrate or solvate of the compound, the stereoisomer or the racemic body, or a prodrug thereof.

[A14]

The compound according to [A12] or [A13], wherein Ar^{B} represents a divalent group obtained by removing two hydrogen atoms from a compound selected from the group consisting of benzene and thiophene,

or a possible stereoisomer or racemic body thereof, or a pharmacologically acceptable salt, hydrate or solvate of the compound, the stereoisomer or the racemic body, or a prodrug thereof.

[A14-2]

The compound according to [A12] or [A13], wherein Ar^{B} represents a divalent group obtained by removing two hydrogen atoms from benzene,

or a possible stereoisomer or racemic body thereof, or a pharmacologically acceptable salt, hydrate or solvate of the compound, the stereoisomer or the racemic body, or a prodrug thereof.

[A15]

The compound described in any one of [A12] to [A14-2], wherein V^{1B} represents a divalent group obtained by removing two hydrogen atoms from a compound selected from the group consisting of [1,2,4]-oxadiazole, [1,3,4]-oxadiazole, [1,2,4]-thiadiazole and [1,3,4]-thiadiazole, or a possible stereoisomer or racemic body thereof, or a pharmacologically acceptable salt, hydrate or solvate of the compound, the stereoisomer or the racemic body, or a prodrug thereof.

[A16]

The compound described in any one of [A12] to [A14-2], wherein V^{1B} represents a single bond; n^{B} denotes 0; and Z^{3B} represents a divalent group obtained by removing two hydrogen atoms from a compound selected from the group consisting of furan, pyrrole, oxazole, thiazole, isothiazole, pyran, pyridine, pyridazine, pyrimidine, pyrazine, oxazine and thiazine,

or a possible stereoisomer or racemic body thereof, or a pharmacologically acceptable salt, hydrate or solvate of the compound, the stereoisomer or the racemic body, or a prodrug thereof.

[A17]

The compound described in anyone of [A12] to [A16], wherein B^{B} represents a C2 alkylene group; and Ar^{B} represents a divalent group obtained by removing two hydrogen atoms from benzene,

or a possible stereoisomer or racemic body thereof, or a pharmacologically acceptable salt, hydrate or solvate of the compound, the stereoisomer or the racemic body, or a prodrug thereof.

[A18]

The compound described in any one of [A12] to [A17], wherein Z^{3B} represents a C3-C6 cycloalkylene group, or a divalent group obtained by removing two hydrogen atoms from a compound selected from the group consisting of benzene, thiophene, oxadiazole, thiazole, isothiazole, thiadiazole, pyran, pyridine, pyridazine, pyrimidine and pyrazine,

or a possible stereoisomer or racemic body thereof, or a pharmacologically acceptable salt, hydrate or solvate of the compound, the stereoisomer or the racemic body, or a prodrug thereof.

[A19]

The compound described in any one of [A12] to [A17], wherein Z^{3B} represents a C3-C6 cycloalkylene group, or a divalent group obtained by removing two hydrogen atoms from a compound selected from the group consisting of benzene, thiophene and pyridine,

or a possible stereoisomer or racemic body thereof, or a pharmacologically acceptable salt, hydrate or solvate of the compound, the stereoisomer or the racemic body, or a prodrug thereof.

[A19-2]

The compound described in any one of [A12] to [A17], wherein Z^{3B} represents a divalent group obtained by removing two hydrogen atoms from a compound selected from the group consisting of benzene and pyridine,

or a possible stereoisomer or racemic body thereof, or a pharmacologically acceptable salt, hydrate or solvate of the compound, the stereoisomer or the racemic body, or a prodrug thereof.

[A20]

The compound according to [A12], wherein

Ar⁸ represents a divalent group obtained by removing two hydrogen atoms from benzene, while the group may be substituted with one to two X^{1B}s and when the group is substituted with two X^{1B}s, X^{1B}s may be the same or different, with X¹B representing a fluorine atom, a chlorine atom, a bromine atom, —OR^{X1B} or —R^{X1B}, and R^{X1B} representing a hydrogen atom, a methyl group, an ethyl group, a propyl group or an isopropyl group;

B^{B} represents a C2 alkylene group;

R^{EB} represents a hydrogen atom, a methyl group, an ethyl group, —(CH₂)_{mB}N(R^{EB1})(R^{EB2}) or —C(R^{EB3})₂OC(O)A^{EB}R^{EB4};

W^{B} represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine, with the proviso that the monovalent group obtained by removing one hydrogen atom from a compound selected from the group consisting of benzene, naphthalene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine may be substituted with one to two X^{4B}s, and when the monovalent group is substituted with two X^{4B}s, X^{4B}s may be the same or different, with X^{4B} representing a fluorine atom, a chlorine atom, a trifluoromethyl group, a trifluoromethoxy group, a cyano group, —OR^{X4B} or —R^{X4B}, and R^{X4B} representing a hydrogen atom, a methyl group, an ethyl group, a propyl group, an isopropyl group, a cyclopentyl group, a cyclohexyl group or a phenyl group;

T^{B} represents a single bond, a methylene group, an ethylene group or —O—;

Z^{3B} represents a divalent group obtained by removing two hydrogen atoms from a compound selected from the group consisting of benzene and pyridine, with the proviso that Z^{3B} may be further substituted with one to four X^{Z3B}s, and when the divalent group is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{X23B}, and R^{XZ3B} representing a hydrogen atom, a C1-C6 alkyl group or a C3-C6 cycloalkyl group, provided that the C1-C6 alkyl group and the C3-C6 cycloalkyl group may be respectively substituted with a fluorine atom(s); and

V^{1B} represents a divalent group obtained by removing two hydrogen atoms from a compound selected from the group consisting of [1,2,4]-oxadiazole, [1,3,4]-oxadiazole, [1,2,4]-thiadiazole and [1,3,4]-thiadiazole,

or a possible stereoisomer or racemic body thereof, or a pharmacologically acceptable salt, hydrate or solvate of the compound, the stereoisomer or the racemic body, or a prodrug thereof.

[A20-2]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or R^{X1B}, and R^{X1B} representing a methyl group or an ethyl group;

B^{B} represents a C2 alkylene group;

R^{E2} represents a hydrogen atom, a methyl group or an ethyl group;

W^{B} represents a monovalent group obtained by removing one hydrogen atom from a compound selected from the group consisting of furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine, while these groups may be substituted with one to two X^{4B}s, and when these groups are substituted with two X^{4B}s, X^{4B}s may be the same or different, with X^{4B} representing a fluorine atom, a chlorine atom, a trifluoromethyl group, a trifluoromethoxy group, a cyano group, —OR^{X4B} or —R^{X4B}, and R^{X4B} representing a hydrogen atom, a methyl group, an ethyl group, a propyl group, an isopropyl group, a cyclopentyl group, or a cyclohexyl group;

T^{B} represents a single bond or —O—;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group, provided that the C1-C6 alkyl group and the C3-C6 cycloalkyl group may be substituted with a fluorine atom(s); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-3]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B}s or —R^{X1B}, and R^{X1B} representing a methyl group or an ethyl group;

B^{B} represents a C2 alkylene group;

R^{E8} represents a hydrogen atom, a methyl group or an ethyl group;

W⁸ represents a C5-C6 alkyl group which may be substituted with 1 to 7 fluorine atoms, or a C5-C7 cycloalkyl group which may be substituted with 1 to 7 fluorine atoms;

T^{B} represents a single bond or —O—;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} s may be the same or different, with X^{Z3B} representing a fluorine atom, a chlorine atom, a trifluoromethyl group, a cyano group, —OR^{XZ3}s, —SR^{XZ3B} or —R^{XZ3B}, and R^{XZ3B} representing a hydrogen atom, a C1-C6 alkyl group or a C3-C6 cycloalkyl group, provided that the C1-C6 alkyl group and the C3-C6 cycloalkyl group may be respectively substituted with a fluorine atom(s); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-4]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B² represents a C2 alkylene group;

R^{EB} represents a hydrogen atom, a methyl group, or an ethyl group;

W^{B} represents a monovalent group obtained by removing one hydrogen atom from benzene, while the group may be substituted with one to two X^{4B}s, and when the group is substituted with two X^{4B}s, X^{4B}s may be the same or different, with X^{4B} representing a trifluoromethoxy group, a cyano group or —OR^{X4B}, and R^{X4B} representing a hydrogen atom, a methyl group or an ethyl group;

T^{B} represents a single bond or —O—;

Z^{3B} represents a divalent group obtained by removing two hydrogen atoms from benzene, with the proviso that Z^{3B} may be further substituted with one to four X^{Z3B}s and when Z^{3B} is substituted with two or more X^{Z3B}s, X^{Z3B} s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-5]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from a compound selected from the group consisting of furan, thiophene and pyridine, while these groups may be substituted with one to two X^{4B}s, and when these groups are substituted with two X^{4B}s, X^{4B}s may be the same or different, with X^{4B} representing a fluorine atom, a trifluoromethyl group, a cyano group or —R^{X4B}, and R^{X4B} representing a methyl group or an ethyl group;

TB represents a single bond or —O—;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-6]

The compound according to [A12], wherein

Ar^{B} represents a divalent group obtained by removing two hydrogen atoms from benzene, while the group may be substituted with one to two X^{1B}s and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from benzene, while the group may be substituted with one to two X^{4B}s, and when the group is substituted with two X^{4B}s, X^{4B}s may be the same or different, with X⁴ representing a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-7]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from benzene, while the group may be substituted with one to two X^{4B}s, and when the group is substituted with two X^{4B}s, X^{4B}s may be the same or different, with X^{4B} representing a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-8]

The compound according to [A12], wherein

Ar^{B} represents a divalent group obtained by removing two hydrogen atoms from benzene and thiophene, while these groups may be substituted with one to two X^{1B}s, and when these groups are substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from benzene, while the group may be substituted with one to two X^{4B}s, and when the group is substituted with two X^{4B}s, X^{4B}s may be the same or different, with X^{4B} representing a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} s may be the same or different, with X^{Z3B} representing a fluorine atom, a chlorine atom, a trifluoromethyl group, a cyano group, —OR^{XZ3B}, —SR^{XZ3B} or R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-9]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from benzene, while the group may be substituted with one to two X^{4B}s, and when the group is substituted with two X^{4B}s, X^{4B}s may be the same or different, with X⁴³ representing a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-10]

The compound according to [A12], wherein

Ar^{B} represents a divalent group obtained by removing two hydrogen atoms from benzene and thiophene, while these groups may be substituted with one to two X^{1B}s, and when these groups are substituted with two X^{1B}s, X^{1B}s may be the same or different, with X^{1B} representing —OR^{X1B} or —R^{X1B}, and R^{X1B} representing a methyl group or an ethyl group;

B^{B} are presents a C2 alkylene group;

R^{EB} represents a hydrogen atom, a methyl group, or an ethyl group;

W^{B} represents a monovalent group obtained by removing one hydrogen atom from a compound selected from the group consisting of furan and thiophene, while these groups may be substituted with one to two X^{4B}s, and when these groups are substituted with two X^{4B}s, X^{4B}s may be the same or different, with X^{4B} representing a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B} s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be further substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-11]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from a compound selected from the group consisting of furan and thiophene, while these groups may be substituted with one to two X^{4B}s, and when these groups are substituted with two X^{4B}Bs, X^{4B}s may be the same or different with X^{4B} representing a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group or the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-12]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from a compound selected from the group consisting of oxazole, isoxazole, oxadiazole, thiazole, isothiazole, thiadiazole, pyridazine, pyrimidine and pyrazine, while these groups may be substituted with one to two X^{4B}s, and when these groups are substituted with two X^{4B}s, X^{4B}s may be the same or different, with X^{4B} representing a fluorine atom, a chlorine atom, a trifluoromethyl group, a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}, and when Z^{3B} is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be further substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-13]

The compound according to [A12], wherein

Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from pyridine, while the group may be substituted with one to two X^{4B}s, and when the group is substituted with two X^{4B}s, X^{4B}s may be the same or different, with X^{4B} representing a fluorine atom, a chlorine atom, a trifluoromethyl group, a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene and thiophene, with the proviso that Z^{3B} may be further substituted with one to four X^{Z3B}s, and when Z^{3B} is substituted with two or more X^{Z3B}s, X^{Z3B}s may be the same or different, with X^{Z3B} representing a fluorine atom, a chlorine atom, a trifluoromethyl group, a cyano group, —OR^{XZ3B}, SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole, or a possible stereoisomer or racemic body thereof, or a pharmacologically acceptable salt, hydrate or solvate of the compound, the stereoisomer or the racemic body, or a prodrug thereof.

[A20-14]

The compound according to [A12], wherein

Ar⁶ represents a divalent group obtained by removing two hydrogen atoms from benzene, while the group may be substituted with one to two X^{1B}s, and when the group is substituted with two X^{1B}s, X^{1B}s may be the same or different, with X^{1B} representing —OR^{X1B} or —R^{X1B}, and R^{X1B} representing a methyl group or an ethyl group;

B^{B} represents a C2 alkylene group;

R^{EB} represents a hydrogen atom, a methyl group, or an ethyl group;

W^{B} represents a monovalent group obtained by removing one hydrogen atom from pyridine, while the group may be substituted with one to two X^{4B}s, and when the group is substituted with two X^{4B}s, X^{4B}s may be the same or different, with X^{4B} representing a fluorine atom, a chlorine atom, a trifluoromethyl group, a trifluoromethoxy group, a cyano group or —OR^{X4B}, and R^{X4B} representing a hydrogen atom, a methyl group or an ethyl group;

T^{B} represents a single bond;

Z^{3B} represents a divalent group obtained by removing two hydrogen atoms from benzene, with the proviso that Z^{3B} may be further substituted with one to four X^{Z3B}s, and when Z^{3B} is substituted with two or more X^{Z3B}s, X^{Z3B} may be the same or different, with X^{Z3B} representing a fluorine atom, a chlorine atom, a trifluoromethyl group, a cyano group, —OR^{XZ3B}, —SR^{XZ3B} or —R^{XZ3B}, and R^{XZ3B} representing a hydrogen atom, a C1-C6 alkyl group or a C3-C6 cycloalkyl group (provided that the C1-C6 alkyl group and the C3-C6 cycloalkyl group may be respectively substituted with a fluorine atom(s)); and

V^{1B} represents a divalent group obtained by removing two hydrogen atoms from [1,2,4]-oxadiazole,

or a possible stereoisomer or racemic body thereof, or a pharmacologically acceptable salt, hydrate or solvate of the compound, the stereoisomer or the racemic body, or a prodrug thereof.

[A20-15]

The compound described in any one of [A20-2] to [A20-14], wherein the binding positions of W^{B}-T^{B}-Z^{3B}-(CR^{6B}R^{7B})ₙ^{B}- and —Ar^{B}—CH₂—NR^{1B}—B^{B}—CO₂R^{EB} with respect to the divalent group obtained by removing two hydrogen atoms from [1,2,4]-oxadiazole are (5,3),

or a possible stereoisomer or racemic body thereof, or a pharmacologically acceptable salt, hydrate or solvate of the compound, the stereoisomer or the racemic body, or a prodrug thereof.

[A21]

A pharmaceutical product comprising, as an active ingredient, the compound described in any one of [A2] to [A20-15], or a possible stereoisomer or racemic body thereof, or a pharmacologically acceptable salt, hydrate or solvate of the compound, the stereoisomer or the racemic body, or a prodrug thereof.

[A22]

An S1P1/Edg1 receptor activating agent comprising, as an active ingredient, the compound described in any one of [A2] to [A20-15], or a pharmacologically acceptable salt thereof.

[A23]

A pharmaceutical product according to any one of [A2] to [A20-15], which is a prophylactic and/or therapeutic agent for autoimmune diseases in a mammal.

[A24]

A method for preventing and/or treating autoimmune diseases in a mammal , the method comprising administering to the mammal including human an effective amount of the compound described in any one of [A2] to [A20-15], or a possible stereoisomer or racemic body thereof, or a pharmacologically acceptable salt, hydrate or solvate of the compound, the stereoisomer or the racemic body, or a prodrug thereof.

A compound represented by the following general formula (1):

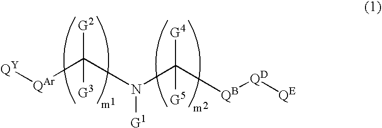

wherein

G¹ represents a hydrogen atom or a C1-C4 alkyl group, with the proviso that the alkyl group may be substituted with one to three X^{G1}s, and when the alkyl group is substituted with two or more X^{G1}s, X^{G1}s may be the same or different;

X^{G1} represents a group selected from the group consisting of —OH, —CO₂H, —SO₃H, —PO₃H₂, and —OPO(OR^{G1})₂;

R^{G2} independently represents a hydrogen atom, or a C1-C4 alkyl group;

G² represents a hydrogen atom, a fluorine atom, a chlorine atom, or a C1-C4 alkyl group, with the proviso that the alkyl group may be substituted with one to three X^{G2}s, and when the alkyl group is substituted with two or more X^{G2}s, X^{G2}s may be the same or different;

X^{G2} represents a group selected from the group consisting of —OH, —CO₂H, —SO₃H, —PO₃H₂, and —OPO(OR^{G2})₂;

R^{G2} independently represents a hydrogen atom, or a C1-C4 alkyl group;

G³ represents a hydrogen atom, a fluorine atom, a chlorine atom, or a C1-C4 alkyl group, with the proviso that the alkyl group may be substituted with one to three X^{G3}s, and when the alkyl group is substituted with two or more X^{G3}s, X^{G3}s may be the same or different;

X^{G3} independently represents a group selected from the group consisting of —OH, —CO₂H, —SO₃H, —PO₃H₂, and —OPO(OR^{G3})₂;

R^{G3} represents a hydrogen atom, or a C1-C4 alkyl group;

G⁴ and G⁵, which may be the same or different, each independently represent a hydrogen atom, a fluorine atom, or a chlorine atom;

Q^{Ar} represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X^{QAr}s, and when these groups are substituted with two X^{QAr}s, X^{QAr}s may be the same or different; XQAr represents a fluorine atom, a chlorine atom, a bromine atom, a trifluoromethyl group, —OR^{XQAr}, —SR^{XQAr}, or —R^{XQAr};

R^{XQAr} represents a hydrogen atom, a C1-C6 alkyl group, a C3-C6 cycloalkyl group, or a phenyl group;

Q^{B} represents B^{Q1}, B^{Q2}, B³ or B4;

B^{Q1} represents a divalent group obtained by removing two hydrogen atoms from a compound selected from the group consisting of a 3- to 8-membered saturated ring compound composed of carbon atoms, a 3- to 8-membered partially unsaturated ring compound composed of carbon atoms, and a 3- to 8-membered unsaturated ring compound composed of carbon atoms, while these groups may be substituted to a possible extent with one to four X^{BQ1}s, and when these groups are substituted with two or more X^{BQ1}s, X^{BQ1}s may be the same or different;

X^{BQ1} represents a group selected from the group consisting of —OH, —CO₂H, —SO₃H, —PO₃H₂, —OPO(OR^{XBQ1})₂, a 1H-tetrazol-5-yl group, a fluorine atom, a chlorine atom, an amino group, -G^{XB1}, —OG^{XB1} and —NG^{XB1}G^{XB1}, or represents a group selected from the group consisting of the following formulas (B3-1) to (B3-5):

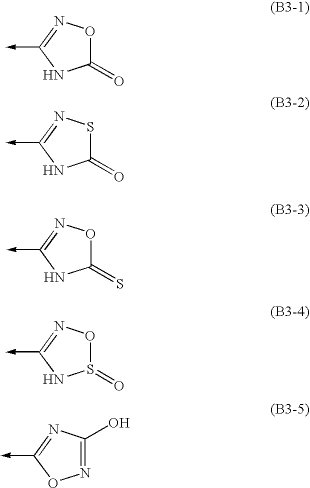

wherein the arrow represents the binding position;

G^{XB1} and G^{XB1}, which may be the same or different, each independently represent a C1-C4 alkyl group which may be substituted with 1 to 5 substituents selected from the group consisting of a halogen atom, a hydroxyl group and an amino group;

R^{XBQ1} independently represents a hydrogen atom, or a C1-C4 alkyl group;

B^{Q2} represents a C1-C4 alkylene group, a C2-C4 alkenylene group, or a C2-C4 alkynylene group, while these groups may be substituted to a possible extent with one to four X^{B2}s, and when these groups are substituted with two or more X^{B2}s, X^{B2}s may be the same or different;

X^{B2} represents a group selected from the group consisting of —OH, —CO₂H, —SO₃ Hr —PO₃H₂, —OPO(OR^{XBQ2})₂, a 1H-tetrazol-5-yl group, a fluorine atom, a chlorine atom, an amino group, -G^{XB2}, —OG^{XB2}, and —NG^{XB2}G^{XB2′};

G^{XB2} and G^{XB2}, which may be the same or different, each independently represent a C1-C4 alkyl group which may be substituted with 1 to 5 substituents selected from the group consisting of a halogen atom, a hydroxyl group and an amino group;

R^{XBQ2} independently represents a hydrogen atom, or a C1-C4 alkyl group;

B³ represents a divalent group obtained by removing two hydrogen atoms from a compound that has a number of ring constituting atoms of 3 to 8, and that is obtained by substituting one to two carbon atoms in the ring of a compound selected from the group consisting of a saturated monocyclic hydrocarbon ring compound, a partially saturated monocyclic hydrocarbon ring compound, and a monocyclic aromatic hydrocarbon ring compound by an oxygen atom, a sulfur atom or a nitrogen atom, while these groups may be substituted to a possible extent with one to four X^{B3}s, and when these groups are substituted with two or more X^{B3}s, X^{B3} s may be the same or different;

X^{B3} represents a group selected from the group consisting of —OH, —CO₂H, —SO₃H, —PO₃H₂, —OPO(OR^{XBQ3})₂, a 1H-tetrazol-5-yl group, a fluorine atom, a chlorine atom, an amino group, -G^{XB3}, —OG^{XB3}, and —NG^{XB3}G^{XB3′};

R^{XBQ3} independently represents a hydrogen atom, or a C1-C4 alkyl group;

G^{XB3} and G^{XB3′}, which may be the same or different, each independently represent a C1-C4 alkyl group which may be substituted with 1 to 5 substituents selected from the group consisting of a halogen atom, a hydroxyl group and an amino group;

B⁴ represents a divalent group obtained by removing two hydrogen atoms from a compound that has a number of ring constituting atoms of 7 to 11, and that is obtained by substituting one to five carbon atoms in the ring of a compound selected from the group consisting of a saturated bicyclic hydrocarbon ring compound, a partially saturated bicyclic hydrocarbon ring compound, and a bicyclic aromatic hydrocarbon ring compound by an oxygen atom, a sulfur atom or a nitrogen atom, while these groups may be substituted to a possible extent with one to four X^{B4}s, and when these groups are substituted with two or more X^{B4}s, X^{B4}s may be the same or different;

X^{B4} represents a group selected from the group consisting of —OH, —CO₂H, —CH₂CO₂H, —SO₃H, —PO₃H₂, —PO₂H₂, —OPO(OR^{XBQ4})₂, a 1H-tetrazol-5-yl group, a fluorine atom, a chlorine atom, an amino group, -G^{XB4}, —OG^{XB4}, and —NG^{XB4}G^{XB4′};

G^{XBQ4} independently represents a hydrogen atom, or a C1-C4 alkyl group;

G^{XB4} and G^{XB4′}, which may be the same or different, each independently represent a C1-C4 alkyl group which may be substituted with 1 to 5 substituents selected from the group consisting of a halogen atom, a hydroxyl group and an amino group;

Q^{D} represents a single bond, or a C1-C3 alkylene group which may be substituted with 1 to 6 fluorine atoms or chlorine atoms;

Q^{E} represents a group selected from the group consisting of —OH, —CO₂R^{QE}, —CH₂CO₂R^{QE}, —SO₃H, —PO₃H₂, —PO₂H₂, —OPO(OR^{QE})₂ and a 1H-tetrazol-5-yl group, or represents a group selected from the group consisting of the following formulas (B3-1) to (B3-5):

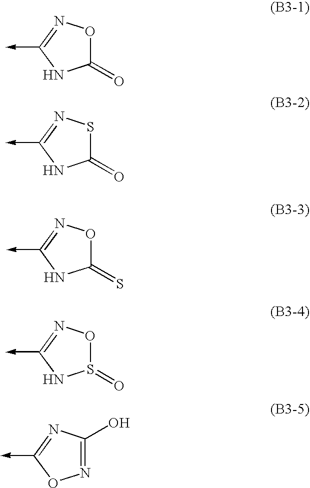

wherein the arrow represents the binding position;

R^{QE} represents a hydrogen atom, a C1-C4 alkyl group, —(CH₂)ₘN(R^{QE1})(R^{QE2}), or —C(R^{QE3})₂OC(O)A^{QE}R^{QE4};

m denotes an integer of 2 or 3;

R^{QE1} and R^{QE2}, which may be the same or different, each independently represent a methyl group, an ethyl group or a propyl group, or R^{QE1} and R^{QE2} may be joined to form a 3- to 6-membered ring together with the nitrogen atom, thus representing a saturated nitrogen-containing cycloalkyl group, or to form a morpholino group together with the nitrogen atom;

R^{QE3} independently represents a hydrogen atom, a methyl group, an ethyl group, or a propyl group;

R^{QE4} represents a C1-C4 alkyl group, a C3-C6 cycloalkyl group, or a phenyl group;

A^{QE} represents a single bond or an oxygen atom;

Q^{Y} represents Q^{W}-Q^{T}-Q^{Z}-(CG⁶G⁷)ₙ-Q^{V}-;

Q^{W} represents a hydrogen atom, a C1-C6 alkyl group which may be substituted with 1 to 7 fluorine atoms, aC3-C7 cycloalkyl group which may be substituted with 1 to 7 fluorine atoms, a C1-C6 alkoxy group which may be substituted with NR^{QW}R^{QW2}, a C3-C7 cycloalkoxy group which may be substituted with NR^{QW}R^{QW2}, or a C1-C6 alkoxy group which may be substituted with a C3-C7 cycloalkyl group in which one to two carbon atoms in the ring may be substituted with a nitrogen atom(s), or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X^{QW}s, and when the monovalent group is substituted with two X^{QW}s, X^{QW}s may be the same or different;

X^{QW} represents a fluorine atom, a chlorine atom, a trifluoromethyl group, —OR^{XQW}, —SR^{XQW}, or —R^{XQW};

R^{XQW} represents a hydrogen atom, a C1-C6 alkyl group, a C3-C6 cycloalkyl group, or a phenyl group;

R^{QW} and R^{QW2}, which may be the same or different, each independently represent a hydrogen atom, or a C1-C4 alkyl group;

Q^{T} represents a single bond, a C1-C6 alkylene group, —O—, —OCH₂—, —S—, —SO—, —SO₂—, —NR^{QT}—, —NR^{QT}NHCO—, or —CONR^{QT}—;

R^{QT} represents a hydrogen atom, or a C1-C4 alkyl group;

Q² is a single bond, or represents a C1-C6 alkylene group or a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that Q^{Z} may be further substituted with one to two X^{QZ}s, and when Q^{Z} is substituted with two X^{QZ}s, X^{Q2}s may be the same or different;

X^{QZ} represents a fluorine atom, a chlorine atom, a trifluoromethyl group, a cyano group, —OR^{XQZ}, —SR^{XQZ}, or —R^{XQZ};

R^{XQZ} represents a hydrogen atom, a C1-C6 alkyl group, a C3-C6 cycloalkyl group, or a phenyl group (provided that the C1-C6 alkyl group, the C3-C6 cycloalkyl group and the phenyl group may be respectively substituted with a fluorine atom);

G⁶ and G⁷, which may be the same or different, each independently represent a hydrogen atom, or a C1-C4 alkyl group which may be substituted with 1 to 5 halogen atoms;

Q^{V} represents a single bond, —CO—, —COCR^{QV}—, —CR^{QV}R^{QV2}—, —S—, —SO—, —SO₂—, —SCH₂—, —SOCH₂—, —SO₂CH₂—, —O—, —CR^{QV}OR^{QV2}—, —CR^{QV}(OR^{QV2})CR^{QV3}R^{QV4}—, —CR^{QV}R^{QV2}CR^{QV3}(OR^{QV4})—, —C≡C—, —CR^{QV}═CR^{QV2}, —NR^{QV}—, —NR^{QV}NHCO—, —CONR^{QV}- or phenylene, or a divalent group obtained by removing two hydrogen atoms from oxadiazole or thiadiazole;

R^{QV}, R^{QV2}, R^{QV3}, and R^{QV4}, which may be the same or different, each independently represent a hydrogen atom, or a C1-C4 alkyl group;

n denotes an integer of 0 to 2, with the proviso that when n denotes 0, it means a single bond;

m¹ denotes an integer of 1 to 3; and

m² denotes an integer of 0 to 3, with the proviso that when m² denotes 0, it means a single bond,

or a possible stereoisomer or racemic body thereof, or a pharmacologically acceptable salt, hydrate or soivate of the compound, the stereoisomer or the racemic body, or a prodrug thereof.

[B2]

A compound represented by the following formula (2):

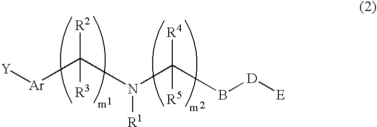

wherein

R¹, R² and R³, which may be the same or different, each independently represent a hydrogen atom, or a C1-C4 alkyl group;

R⁴ and R⁵, which may be the same or different, independently represent a hydrogen atom, a fluorine atom, or a chlorine atom;

Ar represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X¹s, and when these groups are substituted with two X¹s, X¹s may be the same or different;

X¹ represents a fluorine atom, a chlorine atom, a bromine atom, a trifluoromethyl group, —OR^{X1}, —SR^{X1}, or —R^{X1};

R^{X1} represents a hydrogen atom, a C1-C6 alkyl group, a C3-C6 cycloalkyl group, or a phenyl group;

B represents B¹ or B²;

B¹ represents a divalent group obtained by removing two hydrogen atoms from a compound selected from the group consisting of a 3- to 7-membered saturated ring compound composed of carbon atoms, a 3- to 7-membered partially unsaturated ring compound composed of carbon atoms, and a 3- to 7-membered unsaturated ring compound composed of carbon atoms, while these groups may be substituted to a possible extent with one to two X²s, and when these groups are substituted with two X²s, X²s may be the same or different;

X² represents a hydroxyl group, or a carboxyl group;

B² represents a C1-C4 alkylene group, a C2-C4 alkenylene group, or a C2-C4 alkynylene group, while such group may be substituted with one to two X³s, and when the group is substituted with two X³s, X³s may be the same or different;

X³ represents a fluorine atom, a carboxyl group, or a C1-C4 alkyl group which may be substituted with a hydroxyl group or a carboxyl group;

D represents a single bond, a methylene group, or an ethylene group;

E represents a hydroxyl group, —CO₂R^{E}, or a 1H-tetrazol-5-yl group;

R^{E} represents a hydrogen atom, a C1-C4 alkyl group, —(CH₂)ₘN(R^{E1})(R^{E2}), or —C(R^{E3})₂OC(O)A^{E}R^{E4};

m denotes an integer of 2 or 3;

R^{E1} and R^{E2}, which may be the same or different, each independently represent a methyl group, an ethyl group or a propyl group, or R^{E1} and R^{E2} may be joined to form a 3- to 6-membered ring together with the nitrogen atom, thus representing a saturated nitrogen-containing cycloalkyl group, or to form a morpholino group together with the nitrogen atom;

R^{E3} represents a hydrogen atom, a methyl group, an ethyl group or a propyl group;

R^{E4} represents a C1-C4 alkyl group, a C3-C6 cycloalkyl group, or a phenyl group;

A^{E} represents a single bond or an oxygen atom;

Y represents W-T-Z-(CR⁶R⁷)ₙ—V—;

W represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{W}R^{W2}, a C3-C7 cycloalkoxy group which may be substituted with NR_{W}R^{W2}, or a C1-C6 alkoxy group which may be substituted with a C3-C7 cycloalkyl group in which one to two carbon atoms in the ring may be substituted with a nitrogen atom(s), or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X⁴s, and when the monovalent group is substituted with two X⁴s, X⁴s may be the same or different;

X⁴ represents a fluorine atom, a chlorine atom, a trifluoromethyl group, —OR^{X4}, —SR^{X4}, or R^{X4};

R^{X4} represents a hydrogen atom, a C1-C6 alkyl group, a C3-C6 cycloalkyl group, or a phenyl group;

R^{W} and R^{W2}, which may be the same or different, each independently represent a hydrogen atom, or a C1-C6 alkyl group;

T represents a single bond, a C1-C6 alkylene group, —O—, —OCH₂—, —S—, —SO—, —SO₂—, —NR^{T}—, —NR^{T}NHCO—, or CONR^{T}—;

R^{T} represents a hydrogen atom, or a C1-C6 alkyl group;

Z represents a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that Z may be further substituted with one to two X⁵s, and when Z is substituted with two X⁵s, X⁵s may be the same or different;

X⁵ represents a fluorine atom, a chlorine atom, a trifluoromethyl group, a cyano group, —OR^{X5}, —SR^{X5}, or —R^{X5};

R^{X5} represents a hydrogen atom, a C1-C6 alkyl group, a C3-C6 cycloalkyl group, or a phenyl group (provided that the C1-C6 alkyl group, the C3-C6 cycloalkyl group and the phenyl group may be respectively substituted with a fluorine atom;

R⁶ and R⁷, which may be the same or different, each independently represent a hydrogen atom, or a C1-C4 alkyl group;

V represents a single bond, —CO—, —COCR^{V}—, —CR^{V}R^{V2}—, —S—, —SO—, —SO₂—, —SCH₂—, —SOCH₂—, —SO₂CH₂—, —O—, —CR^{V}OR^{V2}—, —CR^{V}(OR^{V2})CR^{V3}R^{V4}—, —CR^{V}R^{V2}CR^{V3}(OR^{V4})—, —C≡C—, —CR^{V}═CR^{V2}, —NR^{V}—, —NR^{V}NHCO— or —CONR^{V}—, or represents a divalent group obtained by removing two hydrogen atoms from oxadiazole or thiadiazole;

R^{V}, R^{V2}, R^{V3} and R^{V4}, which may be the same or different, each independently represent a hydrogen atom, or a C1-C4 alkyl group;

n denotes an integer of 0 to 2, with the proviso that when n denotes 0, it means a single bond;

m¹ denotes an integer of 1 to 3; and

m² denotes an integer of 0 to 3, with the proviso that when m² denotes 0, it means a single bond,

or a possible stereoisomer or racemic body thereof, or a pharmacologically acceptable salt, hydrate or solvate of the compound, the stereoisomer or the racemic body, or a prodrug thereof.

[B3]

A compound represented by the following formula (3B):

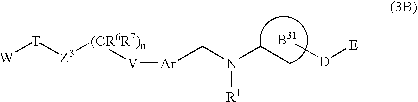

wherein W, T, R⁶, R⁷, n, V, Ar, R¹, D and E have the same meanings as the defined above; B³¹ represents a divalent group obtained by removing two hydrogen atoms from a 3- to 7-membered saturated ring compound composed of carbon atoms; and Z³ represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo(1,2,5)thiadiazole, quinoline, isoquinoline, cinnoline, quinazoline, quinoxaline, phthalazine, and imidazo[1,2-a]pyridine, with the proviso that Z³ may be further substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B4]

A compound represented by the following formula (4):

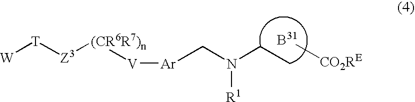

wherein W, T, Z³, R⁶, R⁷, n, V, Ar, R¹, B³¹ and R^{E} have the same meanings as the defined above,

or a possible stereoisomer or racemic body thereof, or a pharmacologically acceptable salt, hydrate or solvate of the compound, the stereoisomer or the racemic body, or a prodrug thereof.

[B5]

A compound represented by the following formula (5):

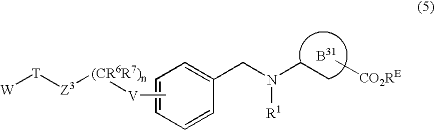

wherein W, T, Z³, R⁶, R⁷, n, V, R¹, B³¹ and R^{E} have the same meanings as the defined above,

or a possible stereoisomer or racemic body thereof, or a pharmacologically acceptable salt, hydrate or solvate of the compound, the stereoisomer or the racemic body, or a prodrug thereof.

[B6]

The compound according to [B5], wherein T is a single bond, a C1-C6 alkylene group, —O—, —OCH₂—, —NR^{T}—, —NR^{T}NHCO—, or —CONR^{T}—; and V represents a single bond, —CO—, —COCR^{V}—, —CR^{V}R^{V2}—, —O—, —CR^{V}OR^{V2}—, —CR^{V}(OR^{V2})CR^{V3}R^{V4}—, —CR^{V}R^{V2}CR^{V3}(OR^{V4})—, —C≡C—, —CR^{V}CR^{V2}—, —NR^{V}—, —NR^{V}NHCO—, or —CONR^{V}—, or is a divalent group obtained by removing two hydrogen atoms from oxadiazole or thiadiazole,

or a possible stereoisomer or racemic body thereof, or a pharmacologically acceptable salt, hydrate or solvate of the compound, the stereoisomer or the racemic body, or a prodrug thereof.

[B7]

The compound described in any one of [B3] to [B6], wherein W represents a hydrogen atom, a C1-C6 alkyl group which may be substituted with 1 to 7 fluorine atoms, or a C3-C7 cycloalkyl group which may be substituted with 1 to 7 fluorine atoms, or is a monovalent group obtained by removing one hydrogen atom from a compound selected from the group consisting of benzene, naphthalene, benzothiophene, thiophene, isoquinoline and indazole, each of which may be substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B8]

The compound according to [B7], wherein W is a monovalent group obtained by removing one hydrogen atom from a compound selected from the group consisting of benzene, naphthalene, benzothiophene, thiophene, isoquinoline and indazole, each of which may be substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B9]

The compound according to [B7], wherein W is a hydrogen atom, a C1-C6 alkyl group which may be substituted with 1 to 7 fluorine atoms, or a C3-C7 cycloalkyl group which may be substituted with 1 to 7 fluorine atoms,

or a possible stereoisomer or racemic body thereof, or a pharmacologically acceptable salt, hydrate or solvate of the compound, the stereoisomer or the racemic body, or a prodrug thereof.

[B10]

The compound described in any one of [B3] to [B6], wherein Z³ represents a C3-C6 cycloalkylene group which may be substituted with one or two groups each independently selected from the group consisting of a fluorine atom, a chlorine atom and a trifluoromethyl group, or is a divalent group obtained by removing two hydrogen atoms from a compound selected from the group consisting of benzene, thiophene, furan, pyridine, oxazole, thiazole, benzothiophene, isoquinoline and indazole, each of which may be substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B11]

The compound according to [B10], wherein Z³ is a divalent group obtained by removing two hydrogen atoms from a compound selected from the group consisting of benzene and thiophene, each of which may be substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B12]

The compound according to [B10] or [B11], wherein Z³ is substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B13]

The compound according to [B2], wherein E is —CO₂R^{E}; B is a 3- to 7-membered saturated ring composed of carbon atoms; V in Y is —O—; W in Y is phenylene; Z in Y is a divalent group obtained by removing two hydrogen atoms from thiophene which may be substituted with one or two groups each independently selected from the group consisting of a fluorine atom, a chlorine atom and a trifluoromethyl group,

or a possible stereoisomer or racemic body thereof, or a pharmacologically acceptable salt, hydrate or solvate of the compound, the stereoisomer or the racemic body, or a prodrug thereof.

[B14]

The compound according to [B13], wherein B is a 3- to 5-membered saturated ring composed of carbon atoms,

or a possible stereoisomer or racemic body thereof, or a pharmacologically acceptable salt, hydrate or solvate of the compound, the stereoisomer or the racemic body, or a prodrug thereof.

[B15]

A pharmaceutical product comprising, as an active ingredient, the compound described in any one of [B2] to [B14], or a possible stereoisomer or racemic body thereof, or a pharmacologically acceptable salt, hydrate or solvate of the compound, the stereoisomer or the racemic body, or a prodrug thereof.

[B16]

An S1P1/Edg1 receptor activating agent comprising, as an active ingredient, the compound described in any one of [B2] to [B14], or a pharmacologically acceptable salt thereof.

[B17]

A pharmaceutical product according to any one of [B2] to [B14], which is a prophylactic and/or therapeutic agent for autoimmune diseases of a mammal.

### EFFECTS OF THE INVENTION

The compound of the present invention, when administered to a human or an animal in a free form or in the form of a salt thereof, has a potent immunosuppressive action, and is useful for a variety of various autoimmune diseases or chronic inflammatory diseases, including systematic erythematodes, chronic rheumatoid arthritis, Type I diabetes, inflammatory bowel disease, biliary cirrhosis, uveitis, multiple sclerosis or other disorders, or for chemotherapy to treat cancers, lymphoma or leukemia.

### BEST MODE FOR CARRYING OUT THE INVENTION

Hereinafter, the present invention will be described in detail.

In the present specification, a carbon atom may be represented simply by “C”, a hydrogen atom by “H”, an oxygen atom by “O”, a sulfur atom by “S”, and a nitrogen atom by “N”. Furthermore, a carbonyl group may be represented simply by “—CO—”, a carboxyl group by “—CO₂—” a sulfinyl group by “—SO—”, a sulfonyl group by “—SO₂—”, an ether bond by “—O—”, and a thioether bond by “—S-” (in these cases, “−” represents a bond).

In the present specification, a C1-C4 alkyl group means an alkyl group having 1 to 4 carbon atoms, which may be straight-chained or branched, and examples thereof include a methyl group, an ethyl group, a propyl group, a butyl group, and isomers thereof [normal (n), iso (iso), secondary (sec), tertiary (t), etc.].

In the present specification, a C1-C6 alkyl group means an alkyl group having 1 to 6 carbon atoms, which may be straight-chained or branched, and examples thereof include a methyl group, an ethyl group, a propyl group, a butyl group, a pentyl group, a hexyl group, and isomers thereof [normal (n), iso (iso), secondary (sec), tertiary (t), etc.].

In the present specification, a C1-C3 alkylene group may be exemplified by a methylene group, an ethylene group, or a trimethylene group.

In the present specification, a C1-C4 alkylene group may be exemplified by a methylene group, an ethylene group, a trimethylene group, a tetra methylene group, or isomers thereof.

In the present specification, a C1-C6 alkylene group may be exemplified by a methylene group, an ethylene group, a trimethylene group, a tetra methylene group, a pentamethylene group, a hexamethylene group, or isomers thereof.

In the present specification, a C2-C4 alkenylene group may be exemplified by an ethenylene group, a propenylene group, a butenylene group, or a butadienylene group.

In the present specification, a C2-C4 alkynylene group may be exemplified by an ethynylene group, a propynylene group, a butynylene group, or a butadiynylene group.

In the present specification, a C3-C6 cycloalkyl group may be exemplified by a cyclopropyl group, a cyclobutyl group, a cyclopentyl group, or a cyclohexyl group.

In the present specification, a C3-C6 cycloalkylene group may be exemplified by a cyclopropylene group, a cyclobutylene group, a cyclopentylene group, or a cyclohexylene group.

In the present specification, a C3-C7 cycloalkyl group may be exemplified by a cyclopropyl group, a cyclobutyl group, a cyclopentyl group, a cyclohexyl group, or a cycloheptyl group.

In the present specification, a C1-C6 alkoxy group may be exemplified by a methoxy group, an ethoxy group, a propoxy group, a butoxy group or the like, or isomers thereof.

A C3-C7 cycloalkoxy group may be exemplified by a cyclopropyloxy group, a cyclobutyloxy group, a cyclopentyloxy group, a cyclohexyloxy group, or a cycloheptyloxy group.

In the present specification, a halogen atom may be exemplified by a fluorine atom, a chlorine atom, a bromine atom, or an iodine atom.

According to the present invention, unless otherwise specified, all isomers are included. For example, an alkyl group, an alkenyl group, an alkynyl group, an alkoxy group, an alkylene group, an alkenylene group and an alkynylene group include straight-chained groups and branched groups. Furthermore, isomers based on double bond, ring or fused ring (E or Z isomers, or cis or trans isomers), isomers based on the presence of asymmetric carbon or the like (R- or S-isomers, isomers based on α- or β-configuration, enantiomers, or diastereomers, etc.), optically active isomer shaving optical rotation (D- or L-isomers, or d- or l-isomers), isomers based on the difference in polarity due to chromatographic separation (more polar isomers or less polar isomers), equilibrium compounds, rotamers, tautomers, or mixtures thereof at arbitrary ratios, or racemic mixtures are all included in the present invention.

A specific example of the isomer based on a ring in the present invention may be a cis-isomer in which the binding relationship between two substituents based on the plane formed by the ring is in the same direction. The corresponding binding relationship may be referred to as a cis relationship. For example, when B31 is bound to —NR1- at the 1-position and to -D-E at the 3-position of a 4-membered saturated ring compound, the structure is expressed as shown in the following formula (I-1). Also, another example may be a trans-isomer in which the binding relationship between two substituents based on the plane formed by the ring is in the opposite direction. The corresponding binding relationship may be referred to as a trans relationship. For example, when B31 is bound to —NR1- at the 1-position and to -D-E at the 3-position of a 4-membered saturated ring compound, the structure is expressed as shown in the following formula (I-2).

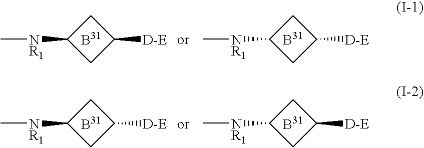

According to the present specification, unless otherwise indicated, as obvious to those having ordinary skill in the art, the symbol:

[symbol]

represents that binding is achieved from the back side of the paper (i.e., α-configuration), the symbol: [symbol]
represents that binding is achieved from the front side of the paper (i.e., β-configuration), the symbol: [symbol]
represents any of the α-configuration or the β-configuration, or a mixture thereof, and the symbol: [symbol]
represents a mixture of the α-configuration and the β-configuration.

The salt of the compound of the present invention is preferably a pharmacologically acceptable salt. If a compound contains a proton-donating substituent, for example, a carboxyl group, a phenolic hydroxyl group or a tetrazole group, a salt in which an arbitrary number of bases is added in accordance with the number of such acidic groups, can be formed. For example, a salt with a metal such as sodium, with an inorganic base such as ammonia, or with an organic base such as triethylamine, may be listed. Furthermore, when a compound contains a substituted or unsubstituted amino group, or contains a basic cyclic structure such as a pyridine ring or a quinoline ring, a salt in which an arbitrary number of acids is added in accordance with the number of such basic groups, is formed. For example, an acid with an inorganic acid such as hydrochloric acid or sulfuric acid, or with an organic acid such as acetic acid or citric acid, may be listed.

Hereinafter, the general formula (1) will be described in detail.

G¹ represents a hydrogen atom, or a C1-C4 alkyl group, with the proviso that the alkyl group may be substituted with one to three X^{G1}s, and when the alkyl group is substituted with two or more X^{G1}s, X^{G1}s may be the same or different;

X^{G1} represents a group selected from the group consisting of —OH, —CO₂H, —SO₃H, —PO₃H₂, and —OPO(OR^{G1})₂; and

R^{G1} independently represents a hydrogen atom, or a C1-C4 alkyl group.

The C1-C4 alkyl group for G¹ is preferably a methyl group or an ethyl group, and particularly preferably a methyl group.

X^{G1} is preferably —OH or —CO₂H, and particularly preferably —OH. Also, —CO₂H may be preferred in some embodiments.

R^{G1} is preferably a hydrogen atom, a methyl group or an ethyl group, more preferably a hydrogen atom or a methyl group, and most preferably a hydrogen atom.

G¹ is preferably a hydrogen atom, or a methyl group or an ethyl group which may be substituted with one of —OH or —CO₂H; more preferably a hydrogen atom, or a methyl group which may be substituted with one of —OH or —CO₂H; still more preferably a hydrogen atom, a methyl group or an ethyl group; even more preferably a hydrogen atom or a methyl group; and most preferably a hydrogen atom.

G² represents a hydrogen atom, a fluorine atom, a chlorine atom, or a C1-C4 alkyl group, with the proviso that the alkyl group may be substituted with one to three X^{G2}s, and when the alkyl group is substituted with two or more X^{G2}s, X^{G2}s may be the same or different;

X^{G2} represents a group selected from the group consisting of —OH, —CO₂H, —SO₃H, —PO₃H₂ and —OPO(OR^{G2})₂; and

R^{G2} independently represents a hydrogen atom, or a C1-C4 alkyl group.

The C1-C4 alkyl group for G² is preferably a methyl group or an ethyl group, and particularly preferably a methyl group.

X^{G2} is preferably —OH or —CO₂H, and particularly preferably —OH. Furthermore, —CO₂H may be preferred in some embodiments.

R^{G2} is preferably a hydrogen atom, a methyl group, or an ethyl group; more preferably a hydrogen atom, or a methyl group; and most preferably a hydrogen atom.

G² is preferably a hydrogen atom, a fluorine atom, or a methyl group which may be substituted with one of —OH or —CO₂H; more preferably a hydrogen atom, or a methyl group which may be substituted with one of —OH or —CO₂H; still more preferably a hydrogen atom, a fluorine atom, or a methyl group; even more preferably a hydrogen atom, or a methyl group; and most preferably a hydrogen atom. Furthermore, a methyl group may be preferred in some embodiments, and in some cases, a fluorine atom may be preferred.

G³ represents a hydrogen atom, a fluorine atom, a chlorine atom, or a C1-C4 alkyl group, with the proviso that the alkyl group may be substituted with one to three X^{G3}s, and when the alkyl group is substituted with two or more X^{G3}s X^{G3}s may be the same or different;

X^{G3} represents a group selected from the group consisting of —OH, —CO₂H, —SO₃H, —PO₃H₂, and OPO OR^{G3})₂; and

R^{G3} independently represents a hydrogen atom or a C1-C4 alkyl group.

The C1-C4 alkyl group for G³ is preferably a methyl group or an ethyl group, and particularly preferably a methyl group.

X^{G3} is preferably —OH or —CO₂H.

R^{G3} is preferably a hydrogen atom, a methyl group, or an ethyl group; more preferably a hydrogen atom or a methyl group; and most preferably a hydrogen atom.

G³ is preferably a hydrogen atom, a fluorine atom, or a methyl group which may be substituted with one of —OH or —CO₂H; more preferably a hydrogen atom, or a methyl group which may be substituted with one of —OH or —CO₂H; still more preferably a hydrogen atom, a fluorine atom, or a methyl group; even more preferably a hydrogen atom or a methyl group; and most preferably a hydrogen atom. Furthermore, a methyl group may be preferred in some embodiments, and in some cases, a fluorine atom may be preferred.

G⁴ and G⁵, which may be the same or different, each independently represent a hydrogen atom, a fluorine atom, or a chlorine atom, but among these, a hydrogen atom or a fluorine atom is preferred, and a hydrogen atom is more preferred.

Q^{Ar} represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X^{QAr}s and when these groups are substituted with two X^{QAr}s, X^{QAr}s may be the same or different;

X^{QAr} represents a fluorine atom, a chlorine atom, a bromine atom, a trifluoromethyl group, —OR^{XQAr}, —SR^{XQAr}, or —R^{XQAr}; and

R^{XQAr} represents a hydrogen atom, a C1-C6 alkyl group, a C3-C6 cycloalkyl group, or a phenyl group.

Specific examples of the compound represented by Q^{Ar}, which is selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, include benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine, imidazo[1,2-a]pyridine, 1H-pyrrolo[2,3-b]pyridine, 1H-pyrrolo[3,2-b]pyridine, 1H-pyrrolo[3,2-c]pyridine, 1H-pyrrolo[2,3-c]pyridine, 1H-pyrazolo[4,3-b]pyridine, 1H-pyrazolo[4,3-c]pyridine, 1H-pyrazolo[3,4-c]pyridine, 1H-pyrazolo[3,4-b]pyridine[1,2,4]triazolo[4,3-a]pyridine, thieno[3,2-c]pyridine, thieno[3,2-b]pyridine, 1H-thieno[3,2-c]pyrazole, 1H-pyrazolo[3,4-d]thiazole[1,2,4]triazolo[1,5-a]pyrimidine, 1H-pyrazolo[3,4-b]pyrazine, 1H-imidazo[4,5-b]pyrazine, 7H-purine[1,8]naphthalidine, or [1,5]naphthalidine, indane, tetrahydronaphthalene, dihydrobenzofuran, 1,3-dioxaindane, chromane, 1,3-dihydrobenzimidazole, dihydro-3H-benzoxazole, dihydro-3H-benzothiazole, dihydro-1H-quinoline, dihydro-2H-isoquinoline, 1,3-dihydropyrrolo[2,3-b]pyridine, and the like.

X^{QAr} is preferably a fluorine atom, a chlorine atom, a bromine atom, a trifluoromethyl group, or —OR^{XQAr}; more preferably a fluorine atom, a chlorine atom, or a trifluoromethyl group; and particularly preferably a fluorine atom. Furthermore, a chlorine atom may be preferred in some embodiments, or a trifluoromethyl group may be preferred in some embodiments.

R^{XQAr} is preferably a hydrogen atom or a C1-C6 alkyl group; more preferably a hydrogen atom, a methyl group, an ethyl group, or a propyl group; and even more preferably a methyl group, an ethyl group, or a propyl group.

Q^{B} represents B^{Q1}, B^{Q2}, B3 or B⁴, but among these, B^{Q1} or B^{Q2} is preferred, and B^{Q1} is even more preferred.

B^{Q1} represents a divalent group obtained by removing two hydrogen atoms from a compound selected from the group consisting of a 3- to 8-membered saturated ring compound composed of carbon atoms, a 3- to 8-membered partially unsaturated ring compound composed of carbon atoms, and a 3- to 8-membered unsaturated ring compound composed of carbon atoms, while these groups may be substituted to a possible extent with one to four X^{BQ1}s, and when these groups are substituted with two or more X^{BQ1}s, X^{BQ1}s may be the same or different;

X^{BQ1} represents a group selected from the group consisting of —OH, —CO₂H, —SO₃H, —PO₃H₂, —OPO(OR^{XBQ1})₂, a 1H-tetrazol-5-yl group, a fluorine atom, a chlorine atom, an amino group, -G^{XB1}, —OG^{XB1}, and —NG^{XB1}G^{XB1′}, or represents a group selected from the group consisting of the following formulas (B3-1) to (B3-5):

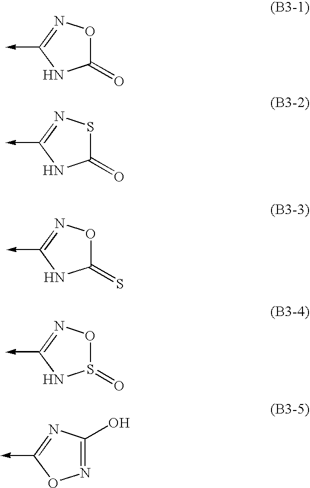

wherein the arrow represents the binding position;

G^{XB1} and G^{XB1′}, which may be the same or different, each independently represent a C1-C4 alkyl group which may be substituted with 1 to 5 substituents selected from the group consisting of a halogen atom, a hydroxyl group and an amino group; and

R^{XBQ1} independently represents a hydrogen atom or a C1-C4 alkyl group.

The compound represented by B^{Q1}, which is selected from the group consisting of a 3- to 8-membered saturated ring compound composed of carbon atoms, a 3- to 8-membered partially unsaturated ring compound composed of carbon atoms, and a 3- to 8-membered unsaturated ring compound composed of carbon atoms, is preferably a 3- to 7-membered saturated ring compound composed of carbon atoms, or a 3- to 7-membered partially unsaturated ring compound composed of carbon atoms, and is preferably a 3- to 7-membered saturated ring compound composed of carbon atoms. Specifically, cyclopropane, cyclobutane, cyclopentane, cyclohexane, cycloheptane, cyclooctane, cyclobutene, cyclopentene, cyclohexene, cycloheptene, cyclooctene, cyclohexadiene, benzene, or pentalene is preferred.

X^{BQ1} is preferably —OH, —CO₂H, a fluorine atom, a chlorine atom, or an amino group; more preferably —OH, a fluorine atom, or an amino group; and most preferably —OH.

R^{XBQ1} is preferably a hydrogen atom, a methyl group, or an ethyl group; more preferably a hydrogen atom, or a methyl group; and most preferably a hydrogen atom.

B^{Q2} represents a C1-C4 alkylene group, a C2-C4 alkenylene group, or a C2-C4 alkynylene group, while such group may be substituted to a possible extent with one to four X^{B2}s, and when the group is substituted with two or more X^{B2}s, X^{B2}s may be the same or different;

X^{B2} represents a group selected from the group consisting of —OH, —CO₂H, —SO₃H, —PO₃H₂, —OPO(OR^{XBQ2})₂, a 1H-tetrazol-5-yl group, a fluorine atom, a chlorine atom, an amino group, -G^{XB2}, —OG^{XB2}, and —NG^{XB2} G^{XB2′};

G^{XB2} and G^{XB2′}, which may be the same or different, each independently represent a C1-C4 alkyl group which may be substituted with 1 to 5 substituents selected from the group consisting of a halogen atom, a hydroxyl group and an amino group; and

R^{XBQ2} independently represents a hydrogen atom or a C1-C4 alkyl group.

B^{Q2} is preferably a C1-C4 alkylene group or a C2-C4 alkenylene group; more preferably a C1-C4 alkylene group; and most preferably an ethylene group.

X^{B2} is preferably —OH, —CO₂H, a fluorine atom, a chlorine atom, or an amino group; more preferably —OH, a fluorine atom, or an amino group; and most preferably —OH.

R^{XBQ2} is preferably a hydrogen atom, a methyl group, or an ethyl group; more preferably a hydrogen atom, or a methyl group; and most preferably a hydrogen atom.

B³ represents a divalent group obtained by removing two hydrogen atoms from a compound that has a number of ring constituting atoms of 3 to 8, and that is obtained by substituting one to two carbon atoms in the ring of a compound selected from the group consisting of a saturated monocyclic hydrocarbon ring compound, a partially saturated monocyclic hydrocarbon ring compound and a monocyclic aromatic hydrocarbon ring compound by an oxygen atom, a sulfur atom or a nitrogen atom, while these groups may be substituted to a possible extent with one to four X^{B3} s, and when these groups are substituted with two or more X^{B3}s, X^{B3}s may be the same or different;

X^{B3} represents a group selected from the group consisting of —OH, —CO₂H, —SO₃H, —PO₃H₂, —OPO(OR^{XBQ3})₂, a 1H-tetrazol-5-yl group, a fluorine atom, a chlorine atom, an amino group, -G^{XB3}, —OG^{XB3}, and —NG^{XB3}G^{XB3′};

R^{XBQ3} independently represents a hydrogen atom or a C1-C4 alkyl group; and

G^{XB3} and G^{XB3′}, which may be the same or different, each independently represent a C1-C4 alkyl group which may be substituted with 1 to 5 substituents selected from the group consisting of a halogen atom, a hydroxyl group and an amino group.

With regard to B³, preferred specific examples of the compound that has a number of ring constituting atoms of 3 to 8, and that is obtained by substituting one to two carbon atoms in the ring of a compound selected from the group consisting of a saturated monocyclic hydrocarbon ring compound, a partially saturated monocyclic hydrocarbon ring compound and a monocyclic aromatic hydrocarbon ring compound by an oxygen atom, a sulfur atom or a nitrogen atom include azilidine, azetidine, pyrrolidine, pyrroline, imidazoline, imidazolidine, pyrazoline, pyrazolidine, piperidine, piperazine, homopiperidine, homopiperazine, azepine, diazepine, morpholine, thiomorpholine, oxolane, thiolane, oxathiane, furan, thiophene, pyrrole, oxazole, isoxazole, thiazole, isothiazole, imidazole, pyrazole, pyran, pyridine, pyridazine, pyrimidine, pyrazine, oxazine or thiazine.

X^{B3} is preferably —OH, —CO₂H, a fluorine atom, a chlorine atom, or an amino group; more preferably —OH, a fluorine atom, or an amino group; and most preferably —OH.

R^{XBQ3} is preferably a hydrogen atom, a methyl group, or an ethyl group; more preferably a hydrogen atom, or a methyl group; and most preferably a hydrogen atom.

B⁴ represents a divalent group obtained by removing two hydrogen atoms from a compound that has a number of ring constituting atoms of 7 to 11, and that is obtained by substituting one to five carbon atoms in the ring of a compound selected from the group consisting of a saturated bicyclic hydrocarbon ring compound, a partially saturated bicyclic hydrocarbon ring compound and a bicyclic aromatic hydrocarbon ring compound by an oxygen atom, a sulfur atom, or a nitrogen atom, while these groups may be substituted to a possible extent with one to four X^{B4}s, and when these groups are substituted with two or more X^{B4}s, X^{B4}s may be the same or different;

X^{B4} represents a group selected from the group consisting of —OH, —CO₂H, —CH₂CO₂H, —SO₃H, —PO₃H₂, —PO₂H₂, —OPO(OR^{XBQ4})₂, a 1H-tetrazol-5-yl group, a fluorine atom, a chlorine atom, an amino group, -G^{XB4}, —OG^{XB4}, and —NG^{XB4}G^{XB4};

R^{XBQ4} independently represents a hydrogen atom or a C1-C4 alkyl group; and

G^{XB4} and G^{XB4′}, which may be the same or different, each independently represent a C1-C4 alkyl group which may be substituted with 1 to 5 substituents selected from the group consisting of a halogen atom, a hydroxyl group and an amino group.

With regard to B⁴, preferred specific examples of the compound that has a number of ring constituting atoms of 7 to 11, and that is obtained by substituting one to five carbon atoms in the ring of a compound selected from the group consisting of a saturated bicyclic hydrocarbon ring compound, a partially saturated bicyclic hydrocarbon ring compound and a bicyclic aromatic hydrocarbon ring compound by an oxygen atom, a sulfur atom or a nitrogen atom include quinuclidine, indoline, isoindoline, dihydrobenzofuran, 1,3-dioxaindane, chromane, 4H-chromene, benzofuran, benzo[b]thiophene, indole, isoindole, indolizine, 1-indazole, 2H-indazole, 1H-benzimidazole, 1,3-dihydrobenzimidazole, benzoxazole, dihydro-3H-benzoxazole, benzo[d]isoxazole, benzo[c]isoxazole, benzothiazole, dihydro-3H-benzothiazole, benzo[d]isothiazole, benzo[c]isothiazole, 1H-benzotriazole, benzo[1,2,5]thiadiazole, quinoline, dihydro-1H-quinoline, isoquinoline, dihydro-2H-isoquinoline, cinnoline, quinazoline, quinoxaline, phthalazine, imidazo[1,2-a]pyridine, 1H-pyrrolo[2,3-b]pyridine, 1H-pyrrolo[3,2-b]pyridine, 1,3-dihydropyrrolo[2,3-b]pyridine, 1H-pyrrolo[3,2-c]pyridine, 1H-pyrrolo[2,3-c]pyridine, 1H-pyrazolo[4,3-b]pyridine, 1H-pyrazolo[4,3-c]pyridine, 1H-pyrazolo[3,4-c]pyridine, 1H-pyrazolo[3,4-b]pyridine, [1,2,4]triazolo[4,3-a]pyridine, thieno[3,2-c]pyridine, thieno[3,2-b]pyridine, 1H-thieno[3,2-c]pyrazole, 1H-pyrazolo[3,4-d]thiazole, [1,2,4]triazolo[1,5-a]pyrimidine, 1H-pyrazolo[3,4-b]pyrazine, 1H-imidazo[4,5-b]pyrazine, 7H-purine[1,8]naphthalidine, or [1,5]naphthalidine.

X^{B4} is preferably —OH, —CO₂H, a fluorine atom, a chlorine atom, or an amino group; more preferably —OH, a fluorine atom, or an amino group; and most preferably —OH.

R^{XBQ4} is preferably a hydrogen atom, a methyl group, or an ethyl group; more preferably a hydrogen atom, or a methyl group; and most preferably a hydrogen atom.

G^{XB1}, G^{XB1′}, G^{XB2}, G^{XB2′}, G^{XB3}, G^{XB3′}, G^{XB4} and G^{XB4′}, which may be the same or different, each independently represent a C1-C4 alkyl group which may be substituted with 1 to 5 substituents selected from the group consisting of a halogen atom, a hydroxyl group and an amino group, but among these, preferred is a methyl group or an ethyl group which may be substituted with 1 to 5 substituents selected from the group consisting of a halogen atom and a hydroxyl group; more preferred is a methyl group or an ethyl group which may be substituted with 1 to 5 substituents selected from the group consisting of a fluorine atom and a hydroxyl group; and most preferred is a methyl group or an ethyl group which may be substituted with one hydroxyl group.

Q^{D} represents a single bond, or a C1-C3 alkylene group which may be substituted with one to six fluorine atoms or chlorine atoms, but among these, preferred is a single bond, a methylene group or an ethylene group; more preferred is a single bond or a methylene group; and most preferred is a single bond.

Q^{E} represents a group selected from the group consisting of —OH, —CO₂R^{QE}, —CH₂CO₂R^{QE}, —SO₃H, —PO₃H₂, —PO₂H₂, —OPO(OR^{QE})₂, and a 1H-tetrazol-5-yl group, or represents a group selected from the following formulas (B3-1) to (B3-5):

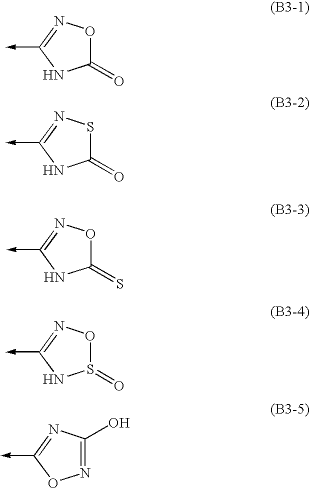

wherein the arrow represents the binding position;

R^{QE} independently represents a hydrogen atom, a C1-C4 alkyl group, —(CH₂)_{mQ}N(R^{QE1})(R^{QE2}), or —C(R^{QE3})₂OC(O)A^{QE}R^{QE4};

m^{Q} denotes an integer of 2 or 3;

R^{QE1} and R^{QE2}, which may be the same or different, each independently represent a methyl group, an ethyl group or a propyl group, or R^{QE1} and R^{QE2} are joined to form a 3- to 6-membered ring together with the nitrogen atom, thus representing a saturated nitrogen-containing cycloalkyl group, or to form a morpholino group together with the nitrogen atom;

R^{QE3} independently represents a hydrogen atom, a methyl group, an ethyl group, or a propyl group;

R^{QE4} represents a C1-C4 alkyl group, a C3-C6 cycloalkyl group, or a phenyl group; and

A^{QE} represents a single bond or an oxygen atom.

Q^{E} is preferably —OH, —CO₂R^{QE}, —CH₂CO₂R^{QE}, —PO₃H₂, —OPO(OR^{QE})₂, or a 1H-tetrazol-5-yl group; more preferably —OH, —CO₂R^{QE}, or a 1H-tetrazol-5-yl group; and most preferably —CO₂R^{QE}.

R^{QE} is preferably a hydrogen atom, a methyl group, an ethyl group, or —(CH₂)_{mq}N(R^{QE1})(R^{QE2}); more preferably a hydrogen atom, a methyl group or an ethyl group; and particularly preferably a hydrogen atom.

Examples of —(CH₂)_{mQ}N(R^{QE1})(R^{QE2}) include a 2-(N,N-dimethylamino)ethyl group, a 2-(N,N-diethylamino)ethyl group, a 2-(N,N-dipropylamino)ethyl group, a 3-(N,N-dimethylamino)propyl group, a 3-(N,N-diethylamino)propyl group, a 2-(N,N-dipropylamino)propyl group, a 2-pyrrolidin-1-ylethyl group, a 2-piperidin-1-ylethyl group, a 2-morpholin-4-ylethyl group, a 3-pyrrolidin-1-ylpropyl group, a 3-piperidin-1-ylpropyl group, a 3-morpholin-4-ylpropyl group, and the like.

Examples of —C(R^{QE3})₂OC(O)A^{QE}R^{QE4} include an acetoxymethyl group, a propionyloxymethyl group, a butylyloxymethyl group, a (2-methylpropionyl)oxymethyl group, a (2,2-dimethylpropionyl)oxymethyl group, a cyclopropionyloxymethyl group, a cyclopentanoyloxymethyl group, a cyclohexanoyloxymethyl group, a phenylcarboxymethyl group, a 1-acetoxy-1-methylethyl group, a 1-methyl-1-(2-methylpropionyloxy)ethyl group, a 1-cyclopentanoyloxy-1-methylethyl group, a 1-cyclohexanoyloxy-1-methylethyl group, a methoxycarbonyloxymethyl group, an ethoxycarbonyloxymethyl group, an isopropyloxycarbonyloxymethyl group, a t-butyloxycarbonyloxymethyl group, a cyclopropyloxycarbonyloxymethyl group, a cyclopentyloxycarbonyloxymethyl group, a cyclohexyloxycarbonyloxymethyl group, a phenyloxycarbonyloxymethyl group, a 1-methoxycarbonyloxy-1-methylethyl group, a 1-ethoxycarbonyloxy-1-methylethyl group, a 1-isopropyloxycarbonyloxy-1-methylethyl group, a 1-t-butyloxycarbonyloxy-1-methylethyl group, a 1-cyclopropyloxycarbonyloxy-1-methylethyl group, a 1-cyclopentyloxycarbonyloxy-1-methylethyl group, a 1-cyclohexyloxycarbonyloxy-1-methylethyl group, or a 1-methyl-1-phenyloxycarbonyloxyethyl group, and the like.

Q^{Y} represents Q^{W}-Q^{T}-Q^{Z}-(CG⁶G⁷)_{nQ}-Q^{V}-;

Q^{W} represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{QW}R^{QW2}, a C3-C7 cycloalkoxy group which may be substituted with NR^{QW}R^{QW2}, or a C1-C6 alkoxy group which may be substituted with a C3-C7 cycloalkyl group in which one to two carbon atoms in the ring may be substituted with a nitrogen atom(s), or a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring may be substituted with one to two X^{QW}s, and when the monovalent group is substituted with two X^{QW}s, X^{QW}s may be the same or different;

X^{QW} represents a fluorine atom, a chlorine atom, a trifluoromethyl group, a trifluoromethoxy group, a cyano group, —OR^{XQW}, —SR^{XQW}, or —R^{XQW};

R^{XQW} represents a hydrogen atom, a C1-C6 alkyl group, a C3-C6 cycloalkyl group, or a phenyl group; and

R^{QW} and R^{QW2}, which may be the same or different, each independently represent a hydrogen atom or a C1-C4 alkyl group.

X^{QW} is preferably a fluorine atom, a chlorine atom, a trifluoromethyl group or —OR^{XQW}; and more preferably a fluorine atom, or a trifluoromethyl group. Apart from these, a fluorine atom, a chlorine atom, a trifluoromethyl group, a trifluoromethoxy group, a cyano group or —OR^{XQW} is preferred; a fluorine atom, a trifluoromethyl group or a cyano group is more preferred; a fluorine atom or a cyano group is even more preferred; and a fluorine atom is particularly preferred. Furthermore, a cyano group may be particularly preferred in some embodiments.

R^{XQW} is preferably a C1-C6 alkyl group or a C3-C6 cycloalkyl group.

R^{QW} and R^{QW2}, which may be the same or different, are each independently preferably a hydrogen atom, a methyl group, or an ethyl group.

Q^{W} is preferably a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, thiophene, benzothiophene, benzofuran, quinoline, tetrahydroquinoline, isoquinoline, tetrahydroisoquinoline, pyridine, indole and indazole; and more preferably a C1-C6 alkyl group which may be substituted with 1 to 7 fluorine atoms, a C3-C7 cycloalkyl group which may be substituted with 1 to 7 fluorine atoms, or a monovalent obtained by removing one hydrogen atom from a compound selected from the group consisting of benzene, naphthalene, thiophene, benzothiophene, isoquinoline and indazole.

Q^{T} represents a single bond, a C1-C6 alkylene group, —O—, —OCH₂—, —S—, —SO—, —SO₂—, —NR^{QT}—, —NR^{QT}NHCO—, or —CONR^{QT}-; and

R^{QT} represents a hydrogen atom or a C1-C4 alkyl group.

Q^{T} is preferably a single bond, —O—, —OCH₂—, or —CONR^{QT}-.

R^{QT} is preferably a hydrogen atom, a methyl group, or an ethyl group.

Q^{Z} is a single bond, or represents a C1-C6 alkylene group or a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that Q^{Z} may be further substituted with one to four x^{QZ}s, there is another embodiment in which QZ is substituted with one to two X^{QZ}s, and X^{QZ} when Q^{Z} is substituted with two or more X^{QZ}s may be the same or different;

X^{QZ} represents a fluorine atom, a chlorine atom, a trifluoromethyl group, a cyano group, —OR^{XQZ}, —SR^{XQZ}, or —R^{XQZ}; and

R^{XQZ} represents a hydrogen atom, a C1-C6 alkyl group, a C3-C6 cycloalkyl group, or a phenyl group (provided that the C1-C8 alkyl group, the C3-C6 cycloalkyl group and the phenyl group may be respectively substituted with a fluorine atom)

X^{QZ} is preferably a fluorine atom, a trifluoromethyl group, a cyano group, or —R^{XQZ}; and more preferably a trifluoromethyl group. Apart from these, a fluorine atom, a trifluoromethyl group, a trifluoromethoxy group, a cyano group, —OR^{XQZ} or —R^{XQZ} is preferred; a fluorine atom, a trifluoromethoxy group, a cyano group, or —R^{XQZ} is more preferred, and a fluorine atom is even more preferred. Furthermore, a trifluoromethoxy group may be more preferred in some embodiments. Furthermore, —R^{XQZ} may be more preferred in some embodiments.

R^{XQZ} is preferably a hydrogen atom or a C1-C6 alkyl group; and more preferably a hydrogen atom, a methyl group, or an ethyl group. Apart from these, a hydrogen atom, a methyl group, an ethyl group, a propyl group, an isopropyl group, a cyclopentyl group, a cyclohexyl group, or a phenyl group is preferred; a methyl group, an ethyl group, or an isopropyl group is more preferred; a methyl group, or an ethyl group is even more preferred; and a methyl group is particularly preferred.

When Q^{Z} is substituted with two or more X^{QZ}s, X^{QZ} is highly preferably a combination of the above-described preferred examples.

Q^{Z} is preferably a single bond, a C1-C6 alkylene group, a C3-C6 cycloalkylene group, or a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, and a bicyclic aromatic heterocyclic compound; more preferably a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine, and imidazo[1,2-a]pyridine; and is more preferably a C5-C6 cycloalkylene group, or a divalent group obtained by removing two hydrogen atoms from benzene, furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, or isoquinoline.

G⁶ and G⁷, which may be the same or different, each independently represent a hydrogen atom, or a C1-C4 alkyl group which may be substituted with 1 to 5 halogen atoms, but among these, are each preferably a hydrogen atom, a methyl group or an ethyl group, and more preferably a hydrogen atom.

Q^{V} represents a single bond, —CO—, —COCR^{QV}—, —CR^{QV}R^{QV2}-, —S—, —SO—, —SO₂—, —SCH₂—, —SOCH₂—, —SO₂CH₂—, —O—, —CR^{QV}OR^{QV2}—, —CR^{QV}(OR^{QV2})CR^{QV3}R^{QV4}, —CR^{QV}R^{QV2}CR^{QV3}(OR^{QV4})—, —C≡C—, —CR^{QV}═CR^{QV2}—, —NR^{QV}-, —NR^{QV}NHCO—, —CONR^{QV}-, or phenylene, or represents a divalent group obtained by removing two hydrogen atoms from oxadiazole or thiadiazole; and

R^{QV}, R^{QV2}, R^{QV3} and R^{QV4}, which may be the same or different, each independently represent a hydrogen atom or a C1-C4 alkyl group.

Q^{V} is preferably a single bond, —CO—, —COCR^{QV}-, —CR^{QV}R^{QV2}-, —S—, —SO—, —SO₂—, —O—, —CR^{QV}(OR^{QV2})CR^{QV3}R^{QV4}-, —CR^{QV}R^{QV2}CR^{QV3}(OR^{QV4})—, —C≡C—, —CR^{QV}═CR^{QV2}- or phenylene, or a divalent group obtained by removing two hydrogen atoms from oxadiazole or thiadiazole; more preferably a single bond, —CO—, —COCR^{QV}—, —CR^{QV}R^{QV2}—, —O—, —CR^{QV}(OR^{QV2})CR^{QV3}R^{QV4}-, CR^{QV}R^{QV2}CR^{QV3}(OR^{QV4})—, —C≡C—, —CR^{QV}∀CR^{QV2}- or phenylene, or a divalent group obtained by removing two hydrogen atoms from oxadiazole or thiadiazole; and even more preferably a single bond, —CO—, —O—, —C≡C—, —CR^{QV}═CR^{QV2}- or phenylene, or a divalent group obtained by removing two hydrogen atoms from oxadiazole or thiadiazole.

The divalent group obtained by removing two hydrogen atoms from oxadiazole or thiadiazole for Q^{V} is preferably a divalent group obtained by removing two hydrogen atoms from [1,2,4]-oxadiazole, [1,3,4]-oxadiazole, [1,2,4]-thiadiazole, or [1,3,4]-thiadiazole; more preferably a divalent group obtained by removing two hydrogen atoms from [1, 2, 4]-oxadiazole or [1,3,4]-thiadiazole; and even more preferably a divalent group obtained by removing two hydrogen atoms from [1,2,4]-oxadiazole.

R^{QV}, R^{QV2}, R^{QV3} and R^{QV4} are each preferably a hydrogen atom, a methyl group, or an ethyl group; and more preferably a hydrogen atom.

n^{Q} denotes an integer of 0 to 2, with the proviso that when n^{Q} denotes 0, it means a single bond. n^{Q} is preferably 0 or 1.

m¹ denotes an integer of 1 to 3, and is preferably 1 or 2, and more preferably 1.

m² denotes an integer of 0 to 3, with the proviso that when m² denotes 0, it means a single bond. m² is preferably 0 or 1, and more preferably 0.

Hereinafter, general formula (2) will be described in detail.

R¹, R² and R³, which may be the same or different, each independently represent a hydrogen atom or a C1-C4 alkyl group, but among these, R¹ is preferably a hydrogen atom, a methyl group or an ethyl group; more preferably a hydrogen atom or a methyl group; and particularly preferably a hydrogen atom. R² and R³ are each preferably a hydrogen atom, a methyl group or an ethyl group; more preferably a hydrogen atom or a methyl group; and particularly preferably a hydrogen atom.

R⁴ and R⁵, which may be the same or different, each independently represent a hydrogen atom, a fluorine atom or a chlorine atom, but among these, a hydrogen atom or a fluorine atom is preferred, and a hydrogen atom is more preferred.

Ar represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X¹s, and when these groups are substituted with two X¹s, X¹s may be the same or different;

X¹ represents a fluorine atom, a chlorine atom, a bromine atom, a trifluoromethyl group, —OR^{X1}, —SR^{X1}, or —R^{X1}; and

R^{X1} represents a hydrogen atom, a C1-C6 alkyl group, a C3-C6 cycloalkyl group, or a phenyl group.

For the basic skeleton represented by Ar, a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound and a bicyclic aromatic heterocyclic compound, is preferred; a divalent group obtained by removing two hydrogen atoms from naphthalene, benzothiophene, quinoline, benzene, isoquinoline, indazole, pyridine, furan or thiophene, is more preferred; and a divalent group obtained by removing two hydrogen atoms from naphthalene, benzene, isoquinoline, indazole, pyridine, furan or thiophene, is even more preferred. Addition to these, for the basic skeleton represented by Ar, a divalent group obtained by removing two hydrogen atoms from a compound selected from the group consisting of benzene, furan, thiophene and pyridine, is preferred; a divalent group obtained by removing two hydrogen atoms from a compound selected from the group consisting of benzene and thiophene, is more preferred; and a divalent group obtained by removing two hydrogen atoms from benzene is even more preferred. Furthermore, a divalent group obtained by removing two hydrogen atoms from thiophene may be more preferred in some embodiments. Moreover, a divalent group obtained by removing two hydrogen atoms from pyridine may be more preferred in some embodiments.

In the general formula (2), preferred examples of the positions at which Ar is bound to Y and to —(CR²R³)m¹—NR¹(CR⁴R⁵)m²—B-D-E, will be presented in the following as (binding position for Y, binding position for (CR²R³)m¹—NR¹(CR⁴R⁵)ₘ²—B-D-E).

When Ar is a divalent group obtained by removing two hydrogen atoms from naphthalene, (1, 4), (1, 5), (2, 5), (2, 6) or (2, 7) is preferred; (1, 4), (1, 5) or (2, 6) is more preferred; and (1, 4) or (2, 6) is even more preferred.

When Ar is a divalent group obtained by removing two hydrogen atoms from benzothiophene or benzofuran, (2, 4), (2, 5), (2, 6), (3, 6), (4, 7), (4, 2), (5, 2), (6, 2), (6, 3) or (7, 4) is preferred; (2, 5), (2, 6), (4, 7), (5, 2), (6, 2), (7, 4) is more preferred; and (2, 6) or (6, 2) is even more preferred.

When Ar is a divalent group obtained by removing two hydrogen atoms from quinoline or tetrahydroquinoline, (2, 6), (2, 7), (3, 6), (3, 7), (4, 8), (5, 8), (6, 2), (6, 3), (7, 2), (7, 3), (8, 4) or (8, 5) is preferred; (2, 6), (3, 7), (5, 6), (6, 2), (7, 3) or (8, 5) is more preferred; and (2, 6), (3, 7), (6, 2) or (7, 3) is even more preferred.

When Ar is a divalent group obtained by removing two hydrogen atoms from benzene, (1, 3) or (1, 4) is preferred; and (1, 4) is more preferred.

When Ar is a divalent group obtained by removing two hydrogen atoms from isoquinoline, (1, 4), (1, 5), (3, 6), (3, 7), (4, 8), (5, 8), (4, 1), (5, 1), (6, 3), (7, 3), (8, 4) or (8, 5) is preferred; (1, 4), (1, 5), (3, 7), (5, 8), (4, 1), (5, 1), (7, 3) or (8, 5) is more preferred; and (1, 4), (1, 5), (3, 7), (4, 1), (5,1) or (7, 3) is even more preferred.

When Ar is a divalent group obtained by removing two hydrogen atoms from indazole, (1, 3), (1, 4), (1, 5), (2, 5), (2, 6), (3, 7), (4, 7), (3, 1), (4, 1), (5, 1), (5, 2), (6, 2), (7, 3) or (7, 4) is preferred; (1, 3), (1, 5), (2, 6), (3, 7), (3, 1), (5, 1), (6, 2) or (7, 3) is more preferred; and (1, 3), (1, 5), (2, 6), (3,1), (5, 1) or (6,2) is even more preferred.

When Ar is a divalent group obtained by removing two hydrogen atoms from pyridine, (2, 4), (2, 5), (2, 6), (3, 5), (3, 6) or (4, 2) is preferred; (2, 5), (2, 6), (3, 5) or (3, 6) is more preferred; and (2, 5), (2, 6) or (3, 6) is even more preferred.

When Ar is a divalent group obtained by removing two hydrogen atoms from thiophene or furan, (2, 4), (2, 5) or (3, 5) is preferred; and (2, 5) is more preferred.

X¹ is preferably a fluorine atom, a chlorine atom, a bromine atom, —OR^{X1} or —R^{X1}; and more preferably a chlorine atom, a bromine atom or —OR^{X1}. Apart from these, X¹ is more preferably a fluorine atom, a bromine atom, —OR^{X1} or —R^{X1}; even more preferably —OR^{X1} or —R^{X1}; and particularly preferably —R^{X1}. Furthermore, a fluorine atom may be particularly preferred in some embodiments. Moreover, a bromine atom may be particularly preferred in some embodiments.

R^{X1} is preferably a hydrogen atom or a C1-C6 alkyl group; more preferably a hydrogen atom, a methyl group, an ethyl group, a propyl group or an isopropyl group; and even more preferably a methyl group or an ethyl group.

When the substitution is made with two or more of X¹, X¹ is highly preferably a combination of the above-described preferred examples.

Furthermore, Ar is preferably a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound and a bicyclic aromatic heterocyclic compound, which may be substituted with one to two of a fluorine atom, a chlorine atom, a bromine atom, a hydroxyl group, a methoxy group, a methyl group or an ethyl group; more preferably a divalent group obtained by removing two hydrogen atoms from a compound selected from the group consisting of naphthalene, benzothiophene, quinoline, benzene, isoquinoline, indazole, pyridine, furan and thiophene, which may be substituted with one to two of a fluorine atom, a chlorine atom, a bromine atom, a hydroxyl group, a methoxy group, a methyl group or an ethyl group; even more preferably a divalent group obtained by removing two hydrogen atoms from a compound selected from the group consisting of naphthalene, benzene, isoquinoline, indazole, pyridine and thiophene, which may be substituted with one to two of a chlorine atom, a bromine atom, a methoxy group, a methyl group or an ethyl group; and particularly preferably a divalent group obtained by removing two hydrogen atoms from a compound selected from the group consisting of naphthalene, benzene and thiophene, which may be substituted with one to two of a chlorine atom, a bromine atom, a methoxy group, a methyl group or an ethyl group.

B represents B¹ or B², but B¹ is preferred.

B1 represents a divalent group obtained by removing two hydrogen atoms from a compound selected from the group consisting of a 3 to 7-membered saturated ring compound composed of carbon atoms, a 3- to 7-membered partially unsaturated ring compound composed of carbon atoms, and a 3- to 7-membered unsaturated ring compound composed of carbon atoms, while these groups may be substituted to a possible extent with one to two X²s, and when these groups are substituted with two X²s, X²s may be the same or different; and

X² represents a hydroxyl group or a carboxyl group.

As the basic skeleton for B¹, a divalent group obtained by removing two hydrogen atoms from a 3- to 7-membered saturated ring compound composed of carbon atoms, or a 3- to 7-membered partially unsaturated ring compound composed of carbon atoms, is preferred; a divalent group obtained by removing two hydrogen atoms from a 3- to 7-membered saturated ring compound composed/of carbon atoms is more preferred; a divalent group obtained by removing two hydrogen atoms from a 4- to 5-membered saturated ring compound composed of carbon atoms is even more preferred; and a divalent group obtained by removing two hydrogen atoms from a 4-membered saturated ring compound composed of carbon atoms is most preferred.

Preferred examples of the positions at which B¹ is bound to —NR¹— and to -D-E will be presented in the following as (binding position for —NR¹—, binding position for -D-E). In the case where B¹ is a divalent group obtained by removing two hydrogen atoms from a 4-membered saturated ring compound composed of carbon atoms, (1, 2) and (1, 3) are preferred; and (1, 3) is more preferred. In the case of a divalent group obtained by removing two hydrogen atoms from a 5-membered saturated ring compound composed of carbon atoms, (1, 2) and (1, 3) are preferred; and (1, 3) is more preferred. In the case of a divalent group obtained by removing two hydrogen atoms from a 6-membered saturated ring compound composed of carbon atoms, (1, 2) and (1, 3) are preferred; and (1, 3) is more preferred. In the case of a divalent group obtained by removing two hydrogen atoms from a 7-membered saturated ring compound composed of carbon atoms, (1, 3) and (1, 4) are preferred; and (1, 3) is more preferred.

Furthermore, the relationship between the bond between B¹ and —NR¹—, and the bond between B¹ and -D-E may be exemplified by a cis relationship or a trans relationship, and a cis relationship is preferred. Also, a trans relationship may be preferred in some embodiments.

For B¹, specific examples of the compound selected from the group consisting of a 3- to 7-membered saturated ring compound composed of carbon atoms, a 3- to 7-membered partially unsaturated ring compound composed of carbon atoms, and a 3- to 7-membered unsaturated ring compound composed of carbon atoms include cyclopropane, cyclobutane, cyclopentane, cyclohexane, cycloheptane, cyclobutene, cyclopentene, cyclohexene, cycloheptene, cyclohexadiene, benzene, and the like.

X² is preferably a hydroxyl group.

B² represents a C1-C4 alkylene group, a C2-C4 alkenylene group or a C2-C4 alkynylene group, while such group may be substituted with one to two X³s, and when the group is substituted with two X³s, X³s may be the same or different; and

X³ represents a fluorine atom, a carboxyl group, or a C1-C4 alkyl group which may be substituted with a hydroxyl group or a carboxyl group.

The main chain of B² is preferably a C1-C4 alkylene group or a C2-C4 alkylene group; more preferably a C1-C4 alkylene group; and particularly preferably an ethylene group.

X³ is preferably a methyl group or an ethyl group, which may be substituted with a hydroxyl group or a carboxyl group.

D represents a single bond, a methylene group or an ethylene group; but among these, a single bond or a methylene group is preferred, and a single bond is more preferred.

E represents a hydroxyl group, —CO₂R^{E}, or a 1H-tetrazol-5-yl group;

R^{E} represents a hydrogen atom, a C1-C4 alkyl group, —(CH₂)ₘN(R^{E1})(R^{E2}), or —C(R^{E3})₂OC(O)A^{E}R^{E4};

m denotes an integer of 2 or 3;

R^{E1} and R^{E2}, which may be the same or different, each independently represent a methyl group, an ethyl group or a propyl group, or R^{E1} and R^{E2} are joined to form a 3- to 6-membered ring together with the nitrogen atom, thus representing a saturated nitrogen-containing cycloalkyl group, or to form a morpholino group together with the nitrogen atom;

R^{E3} represents a hydrogen atom, a methyl group, an ethyl group, or a propyl group;

R^{E4} represents a C1-C4 alkyl group, a C3-C6 cycloalkyl group, or a phenyl group; and

A^{E} represents a single bond or an oxygen atom.

E is preferably a hydroxyl group or —CO₂R^{E}, and particularly preferably —CO₂R^{E}.

R^{E} is preferably a hydrogen atom, a methyl group, an ethyl group or —(CH₂)ₘN(R^{E1})(R^{E2}); more preferably a hydrogen atom, a methyl group or an ethyl group; and particularly preferably a hydrogen atom. Examples of —(CH₂)ₘN(R^{E1})(R^{E2}) include a 2-(N,N-dimethylamino)ethyl group, a 2-(N,N-diethylamino)ethyl group, a 2-(N,N-dipropylamino)ethyl group, a 3-(N,N-dimethylamino)propyl group, a 3-(N,N-diethylamino)propyl group, a 2-(N,N-dipropylamino)propyl group, a 2-pyrrolidin-1-ylethyl group, a 2-piperidin-1-lyethyl group, a 2-morpholin-4-ylethyl group, a 3-pyrrolidin-1-ylpropyl group, a 3-piperidin-1-ylpropyl group, a 3-morpholin-4-ylpropyl group, and the like.

Examples of —C(R^{E3})₂OC(O)A^{E}R^{E4} include an acetoxymethyl group, a propionyloxymethyl group, a butyryloxymethyl group, a (2-methylpropionyl)oxymethyl group, a (2,2-dimethylpropionyl)oxymethyl group, a cyclopropionyloxymethyl group, a cyclopentanoyloxymethyl group, a cyclohexanoyloxymethyl group, a phenylcarboxymethyl group, a 1-acetoxy-1-methylethyl group, a 1-methyl-1-(2-methylpropionyloxy)ethyl group, a 1-cyclopentanoyloxy-1-methylethyl group, a 1-cyclohexanoyloxy-1-methylethyl group, a methoxycarbonyloxymethyl group, an ethoxycarbonyloxymethyl group, an isopropyloxycarbonyloxymethyl group, a t-butyloxycarbonyloxymethyl group, a cyclopropyloxycarbonyloxymethyl group, a cyclopentyloxycarbonyloxymethyl group, a cyclohexyloxycarbonyloxymethyl group, a phenyloxycarbonyloxymethyl group, a 1-methoxycarbonyloxy-1-methylethyl group, a 1-ethoxycarbonyloxy-1-methylethyl group, a 1-isopropyloxycarbonyloxy-1-methylethyl group, a 1-t-butyloxycarbonyloxy-1-methylethyl group, a 1-cyclopropyloxycarbonyloxy-1-methylethyl group, a 1-cyclopentyloxycarbonyloxy-1-methylethyl group, a 1-cyclohexyloxycarbonyloxy-1-methylethyl group, or a 1-methyl-1-phenyloxycarbonyloxyethyl group, and the like.

Y represents W-T-Z-(CR⁶R⁷)ₙ—V—.

W represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{W}R^{W2}, a C3-C7 cycloalkoxy group which may be substituted with NR^{W}R^{W2}, or a C1-C6 alkoxy group which may be substituted with a C3-C7 cycloalkyl group in which one to two carbon atoms in the ring may be substituted with a nitrogen atom(s), or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X⁴s, and when the monovalent group is substituted with two X⁴s, X⁴s may be the same or different;

X⁴ represents a fluorine atom, a chlorine atom, a trifluoromethyl group, a trifluoromethoxy group, a cyano group, —OR^{X4}, —SR^{X4} or —R^{X4}—;

R^{X4} represents a hydrogen atom, a C1-C6 alkyl group, a C3-C6 cycloalkyl group, or a phenyl group; and

R^{W} and R^{W2}, which may be the same or different, each independently represent a hydrogen atom or a C1-C6 alkyl group.

X⁴ is preferably a fluorine atom, a trifluoromethyl group, —OR^{X4} or R^{X4}. Apart from these, X⁴ is preferably a fluorine atom, a chlorine atom, a trifluoromethyl group, a trifluoromethoxy group, a cyano group, or —ORX4; more preferably a fluorine atom, a trifluoromethyl group, or a cyano group; even more preferably a fluorine atom or a cyano group; and particularly preferably a fluorine atom. A cyano group may be particularly preferred in some embodiments.

R^{X4} is preferably a hydrogen atom, a methyl group, an ethyl group, a propyl group, an isopropyl group, a cyclopentyl group, a cyclohexyl group, or a phenyl group; and more preferably a methyl group, an ethyl group, or a propyl group.

R^{W} and R^{W2} are each preferably a hydrogen atom, a methyl group, or an ethyl group; and more preferably a hydrogen atom.

W is preferably a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, thiophene, benzothiophene, benzofuran, quinoline, tetrahydroquinoline, isoquinoline, tetrahydroisoquinoline, pyridine, indole and indazole; more preferably a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, thiophene, benzothiophene, isoquinoline and indazole; and even more preferably a C1-C6 alkyl group which may be substituted with 1 to 7 fluorine atoms, or a C3-C7 cycloalkyl group which may be substituted with 1 to 7 fluorine atoms. A monovalent group obtained by removing one hydrogen atom from a compound selected from the group consisting of benzene, naphthalene, thiophene, benzothiophene, isoquinoline and indazole, may be more preferred in some embodiments.

Apart from these, W is preferably a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene and pyridine; more preferably a C1-C6 alkyl group which may be substituted with 1 to 7 fluorine atoms, or a C3-C7 cycloalkyl group which may be substituted with 1 to 7 fluorine atoms; and even more preferably a C5-C6 alkyl group which may be substituted with 1 to 7 fluorine atoms, or a C5-C7 cycloalkyl group which may be substituted with 1 to 7 fluorine atoms. Furthermore, a monovalent group obtained by removing one hydrogen atom from a compound selected from the group consisting of benzene and thiophene, may be more preferred in some embodiments. Also, a monovalent group obtained by removing one hydrogen atom from pyridine, may be more preferred in some embodiments. Moreover, a monovalent group obtained by removing one hydrogen atom from benzene may be more preferred in some embodiments.

Furthermore, a monovalent group obtained by removing one hydrogen atom from a compound selected from the group consisting of benzene, naphthalene, thiophene, benzothiophene, benzofuran, quinoline, tetrahydroquinoline, isoquinoline, tetrahydroisoquinoline, pyridine, indole and indazole, which is substituted with the preferred examples of X⁴ or R^{X4} aforementioned, is preferred; and a monovalent group obtained by removing one hydrogen atom from a compound selected from the group consisting of benzene, naphthalene, thiophene, benzothiophene, isoquinoline and indazole, which is substituted with the preferred examples of X⁴ or R^{X4} aforementioned, may be more preferred in some embodiments.

T represents a single bond, a C1-C6 alkylene group, —O—, —OCH₂—, —S—, —SO—, —SO₂—, —NR^{T}—, —NR^{T}NHCO—, or —CONR^{T}—;

R^{T} represents a hydrogen atom, or a C1-C6 alkyl group.

T is preferably a single bond, a C1-C6 alkylene group, —O—, —OCH₂—, —NR^{T}—, —NR^{T}NHCO— or —CONR^{T}—; more preferably a single bond, a methylene group, an ethylene group or —O—; and even more preferably a single bond or —O—. —CONR^{T}— may be more preferred in some embodiments.

R^{T} is preferably a hydrogen atom, a methyl group, or a C4-C6 alkyl group; and more preferably a hydrogen atom or a methyl group. A C4-C6 alkyl group may be more preferred in some embodiments.

Z represents a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that Z may be further substituted with one to four X⁵s, and more preferably may be substituted with one to two X⁵s, and when Z is substituted with two or more X⁵s, X⁵s may be the same or different;

X⁵ represents a fluorine atom, a chlorine atom, a trifluoromethyl group, a cyano group, —OR^{X5}, —SR^{X5}, or —R^{X5}; and

R^{X5} represents a hydrogen atom, a C1-C6 alkyl group, a C3-C6 cycloalkyl group, or a phenyl group (provided that the C1-C6 alkyl group, the C3-C6 cycloalkyl group and the phenyl group may be respectively substituted with a fluorine atom)

X⁵ is preferably a fluorine atom, a trifluoromethyl group, a cyano group, —OR^{X5}, or —R^{X5}; more preferably a trifluoromethyl group, or —R^{X5}; and particularly preferably a trifluoromethyl group.

Apart from these, X⁵ is preferably a fluorine atom, a chlorine atom, a trifluoromethyl group, a trifluoromethoxy group, a cyano group, —OR^{X5}, or —R^{X5}; more preferably a fluorine atom, a trifluoromethoxy group, a cyano group, or —R^{X5}; even more preferably a fluorine atom, a cyano group, or —R^{X5}; and particularly preferably a fluorine atom or a cyano group.

Furthermore, a trifluoromethoxy group may be particularly preferred in some embodiments. Moreover, —R^{X5} may be particularly preferred in some embodiments.

R^{X5} is preferably a hydrogen atom, a methyl group, an ethyl group, a propyl group, an isopropyl group, a cyclopentyl group, a cyclohexyl group, or a phenyl group.

Apart from these, R^{X5} is preferably a hydrogen atom, or a C1-C6 alkyl group; more preferably a methyl group, an ethyl group, or a propyl group; even more preferably a methyl group or an ethyl group; and particularly preferably a methyl group. Furthermore, a methyl group substituted with a fluorine atom may be particularly preferred in some embodiments.

When the substitution is made with two or more X⁵s, X⁵s may be highly preferably a combination of the above-described preferred examples.

Z is preferably a C3-C6 cycloalkylene group, or a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound and a bicyclic aromatic heterocyclic compound; more preferably a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine and imidazo[1,2-a]pyridine; even more preferably a C5-C6 cycloalkylene group, or a divalent group obtained by removing two hydrogen atoms from benzene, furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline or isoquinoline; and even more preferably a C5-C6 cycloalkylene group, or a divalent group obtained by removing two hydrogen atoms from furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, benzo[d]isoxazole, benzo[c]isoxazole, benzo[d]isothiazole, benzo[c]isothiazole, 1H-benzotriazole, benzo[1,2,5]thiadiazole, quinoline or isoquinoline.

Furthermore, the preferred examples of Z which have been substituted with the preferred X⁵ or R^{X5} are also highly preferred.

R⁶ and R⁷, which may be the same or different, each independently represent a hydrogen atom or a C1-C4 alkyl group, but among these, a hydrogen atom, a methyl group or an ethyl group is preferred, and a hydrogen atom is more preferred.

V represents a single bond, —CO—, —COCR^{V}—, —CR^{V}R^{V2}, —S—, —SO—, —SO₂—, —SCH₂—, —SOCH₂—, —SO₂CH₂—, —O—, —CR^{V}OR^{V2}—, —CR^{V}(OR^{V2})CR^{V3}R^{V4}—, —CR^{V}R^{V2}CR^{V3}(OR^{V4})—, —C≡C—, —CR^{V}═CR^{V}═CR^{V2}—, —NR^{V}—, —NR^{V}NHCO— or —CONR^{V}—, or a divalent group obtained by removing two hydrogen atoms from oxadiazole or thiadiazole; and

R^{V}, R^{V2}, R^{V3} and R^{V4}, which may be the same or different, each independently represent a hydrogen atom or a C1-C4 alkyl group.

V is preferably a single bond, —CO—, —COCR^{V}—, —CR^{V}R^{V2}—, —S—, —SO—, —SO₂—, —O—, —CR^{V}(OR^{V2})CR^{V3}R^{V4}—, —CR^{V}CR^{V2}CR^{V3}(OR^{V4})—, —C≡C— or —CR^{V}═CR^{V2}—, or a divalent group obtained by removing two hydrogen atoms from oxadiazole or thiadiazole; more preferably a single bond, —CO—, —COCR^{V}—, —CR^{V}R^{V2}—, —O—, —CR^{V}(OR^{V2})CR^{V3}R^{V4}—, —CR^{V}R^{V2}CR^{V3}(OR^{V4})—, —C≡C— or —CR^{V}═CR²—, or a divalent group obtained by removing two hydrogen atoms from oxadiazole or thiadiazole; even more preferably a single bond, —CO—, —O—, —CR^{V}(OCR^{V2})CR^{V3}CR^{V4}, —C≡C— or —CR^{V}═CR^{V2}—, or a divalent group obtained by removing two hydrogen atoms from oxadiazole or thiadiazole; and still more preferably a single bond, —CO—, —O—, —C≡C— or —CR^{V}═CR^{V2}—, or a divalent group obtained by removing two hydrogen atoms from oxadiazole or thiadiazole.

For V, the divalent group obtained by removing two hydrogen atoms from oxadiazole or thiadiazole is preferably a divalent group obtained by removing two hydrogen atoms from [1,2,4]-oxadiazole, [1,3,4]-oxadiazole, [1,2,4]-thiadiazole or [1,3,4]-thiadiazole; more preferably a divalent group obtained by removing two hydrogen atoms from [1,2,4]-oxadiazole or [1,3,4]-thiadiazole; and even more preferably a divalent group obtained by removing two hydrogen atoms from [1,2,4]-oxadiazole.

In the case where V is a divalent group obtained by removing two hydrogen atoms from a compound selected from the group consisting of oxadiazole and thiadiazole, preferred examples of the positions at which V is bound to W-T-Z-(CR⁶R⁷)ₙ— and to —Ar— will be presented in the following as (binding position for W-T-Z-(CR⁶R⁷)ₙ—, binding positions for —Ar—).

In the case where V is a divalent group obtained by removing two hydrogen atoms from [2, 4]-oxadiazole, (5, 3) is preferred. Furthermore, (3, 5) may be preferred in some embodiments. The expression “the positions at which V is bound to W-T-Z-(CR⁶R⁷)ₙ— and to —Ar— are the 5-position and 3-position of V, respectively” may be used in place of the expression (5,3).

In the case where V is a divalent group obtained by removing two hydrogen atoms from [1, 2, 4]-thiadiazole, (5, 3) is preferred. Furthermore, (3, 5) may be preferred in some embodiments. The expression “the positions at which V is bound to W-T-Z-(CR⁶R⁷)ₙ— and to —Ar— are the 5-position and 3-position of V, respectively” may be used in place of the expression (5,3).

R^{V}, R^{V2}, R^{V3} and R^{V4}, which may be the same or different, are each independently preferably a hydrogen atom, a methyl group or an ethyl group; more preferably a hydrogen atom or a methyl group; and particularly preferably a hydrogen atom.

Furthermore, the preferred examples of V having the preferred R^{V}, R^{V2}, R^{V3} or R^{V4} are also highly preferred.

n denotes an integer of 0 to 2, with the proviso that when n denotes 0, it means a single bond. n is preferably 0 or 1.

m¹ denotes an integer of 1 to 3, and is preferably 1 or 2, and more preferably 1.

m² denotes an integer of 0 to 3, with the proviso that when m2 denotes 0, it means a single bond. m2 is preferably 0 or 1, and more preferably 0.

Hereinafter, general formula (3) will be described in detail.

In the general formula (3), W, T, R⁶, R⁷, n, Ar, R¹, D and E have the same meanings as the aforementioned.

B³¹ represents a divalent group obtained by removing two hydrogen atoms from a 3- to 7-membered saturated ring compound composed of carbon atoms.

B³¹ is preferably a divalent group obtained by removing two hydrogen atoms from a 4- to 5-membered saturated ring compound composed of carbon atoms; and more preferably a divalent group obtained by removing two hydrogen atoms from a 4-membered saturated ring compound composed of carbon atoms.

Preferred examples of the positions at which B³¹ is bound to —NR¹— and to -D-E will be presented in the following as (binding position for —NR¹—, binding position for -D-E).

In the case where B³¹ is a divalent group obtained by removing two hydrogen atoms from a 4-membered saturated ring compound composed of carbon atoms, (1, 2) and (1, 3) are preferred; and (1, 3) is more preferred. In the case of a divalent group obtained by removing two hydrogen atoms from a 5-membered saturated ring compound composed of carbon atoms, (1, 2) and (1, 3) are preferred, and (1, 3) is more preferred. In the case of a divalent group obtained by removing two hydrogen atoms from a 6-membered saturated ring compound composed of carbon atoms, (1, 2) and (1, 3) are preferred, and (1, 3) is more preferred. In the case of a divalent group obtained by removing two hydrogen atoms from a 7-membered saturated ring compound composed of carbon atoms, (1, 3) and (1, 4) are preferred, and (1, 3) is more preferred. The expression “the positions at which B³¹ is bound to —NR¹— and to -D-E are the 1-position and 3-position, respectively” may be used in place of the expression (1,3).

Furthermore, the relationship between the bond between B¹³ and —NR¹—, and the bond between B¹³ and -D-E may be exemplified by a cis relationship or a trans relationship, and a cis relationship is preferred. Also, a trans relationship may be preferred in some embodiments.

Z³ represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine and imidazo[1,2-a]pyridine, with the proviso that Z³ may be further substituted with one to four X^{Z3}s, and Z³ is substituted with one to two X^{Z3}s in some embodiments, and when Z³ is substituted with two or more X^{Z3}s, X^{Z3}s may be the same or different;

X^{Z3} represents a fluorine atom, a chlorine atom, a trifluoromethyl group, a cyano group, —OR^{XZ3}, —SR^{XZ3}, or —R^{XZ3}; and

R^{XZ3} represents a hydrogen atom, a C1-C6 alkyl group, a C3-C6 cycloalkyl group, or a phenyl group (provided that the C1-C6 alkyl group, the C3-C6 cycloalkyl group and the phenyl group may be respectively substituted with a fluorine atom).

X²³ is preferably a fluorine atom, a chlorine atom, a trifluoromethyl group, a trifluoromethoxy group, a cyano group, —OR^{XZ3} or —R^{XZ3}; more preferably a fluorine atom, a trifluoromethoxy group, a cyano group, or —R^{XZ3}; even more preferably a fluorine atom, a cyano group or —R^{XZ3}; and particularly preferably a fluorine atom or a cyano group. Furthermore, a trifluoromethoxy group may be particularly preferred in some embodiments. Moreover, R^{XZ3} may be particularly preferred in some embodiments.

R^{XZ3} is preferably a hydrogen atom or a C1-C6 alkyl group; more preferably a methyl group, an ethyl group or a propyl group; even more preferably a methyl group or an ethyl group; and particularly preferably a methyl group. Furthermore, a methyl group substituted with a fluorine atom may be particularly preferred in some embodiments.

When the substitution is made with two or more X^{Z3}s, X^{Z3}s may be highly preferably a combination of the above-described preferred examples.

Z³ is preferably a C5-C6 cycloalkylene group, or a divalent group obtained by removing two hydrogen atoms from benzene, furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline or isoquinoline; more preferably a C5-C6 cycloalkylene group, or a divalent group obtained by removing two hydrogen atoms from furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, benzo[d]isoxazole, benzo[d]isoxazole, benzo[d]isothiazole, benzo[c]isothiazole, 1H-benzotriazole, benzo[1,2,5]thiadiazole, quinoline or isoquinoline; even more preferably a C5-C6 cycloalkylene group, or a divalent group obtained by removing two hydrogen atoms from furan, thiophene, oxazole, isoxazole, oxadiazole, thiazole, isothiazole, thiadiazole, imidazole, pyrazole, pyran, pyridine, pyrimidine, pyrazine, naphthalene, benzofuran, benzo[b]thiophene, indole, 1H-indazole, 2H-indazole, benzo[d]isoxazole, benzo[c] isoxazole, benzo[d]isothiazole, benzo[c]isothiazole, benzo[1,2,5]thiadiazole, quinoline or isoquinoline; still more preferably a divalent group obtained by removing two hydrogen atoms from thiophene, oxazole, oxadiazole, thiazole, thiadiazole, pyridine, naphthalene, benzo[b]thiophene, indole, 1H-indazole, 2H-indazole, quinoline or isoquinoline; and most preferably a divalent group obtained by removing two hydrogen atoms from thiophene, oxazole, oxadiazole, pyridine, naphthalene, benzo[b]thiophene, 1H-indazole or 2H-indazole. Furthermore, benzene or thiophene may be most preferred in some embodiments, and also, benzene may be most preferred in some embodiments.

The preferred examples of Z³ substituted with the aforementioned preferred X^{Z3}, are also highly preferred.

V¹ represents a single bond, —CO—, COCR^{V1}R^{V12}—, —CR^{V1}R^{V12}—; —O—, —CR^{V1}OR^{V12}—, —CR^{V1}(OR^{V12})CR^{V13}R^{V14}—, —CR^{V1}R^{V12} CR^{V13}(OR^{V14})—, —C≡C—, CR^{V1}═CR^{V12}—, NR^{V1}—, —NR^{V1}NHCO— or —CONR^{V1}—, or a divalent group obtained by removing two hydrogen atoms from oxadiazole or thiadiazole; and

R^{V1}, R^{V12}, R^{V13} and R^{V14}, which may be the same or different, each independently represent a hydrogen atom or a C1-C4 alkyl group.

V¹ is preferably a single bond, —CO—, —CR^{V1}R^{V12}—, —O—, —CR^{V1}(OR^{V12})CR^{V13}R^{V14}—, —CR^{V1}R^{V2}CR^{V13}(OR^{V4})—, —C≡C— or —CR^{V1}═CR^{V12}—, or a divalent group obtained by removing two hydrogen atoms from oxadiazole or thiadiazole; more preferably a single bond, —CO—, —O—, —C≡C— or —CR^{V1}═CR^{V12}, or a divalent group obtained by removing two hydrogen atoms from oxadiazole or thiadiazole; even more preferably a single bond or —O—, or a divalent group obtained by removing two hydrogen atoms from oxadiazole or thiadiazole; particularly preferably a divalent group obtained by removing two hydrogen atoms from oxadiazole or thiadiazole; and still more preferably a divalent group obtained by removing two hydrogen atoms from oxadiazole.

The divalent group obtained by removing two hydrogen atoms from oxadiazole for V¹ is preferably a divalent group obtained by removing two hydrogen atoms from [1,2,4]-oxadiazole or [1,3,4]-oxadiazole; and more preferably a divalent group obtained by removing two hydrogen atoms from [1,2,4]-oxadiazole.

The divalent group obtained by removing two hydrogen atoms from thiadiazole for V¹ is preferably a divalent group obtained by removing two hydrogen atoms from [1,2,4]-thiadiazole or [1,3,4]-thiadiazole; and more preferably a divalent group obtained by removing two hydrogen atoms from [1,3,4]-thiadiazole.

In the case where V¹ is a divalent group obtained by removing two hydrogen atoms from a compound selected from the group consisting of oxadiazole and thiadiazole, preferred examples of the positions at which V¹ is bound to W-T-Z³-(CR⁶R⁷)ₙ— and to —Ar—CH₂—NR¹—B^{B31}-D-E will be presented in the following as (binding position for W-T-Z³-(CR⁶R⁷)ₙ—, binding position for —Ar—CH₂—NR¹—B^{B31}-D-E).

In the case where V¹ is a divalent group obtained by removing two hydrogen atoms from [1,2,4]-oxadiazole, (5, 3) is preferred, and (3, 5) may be preferred in some embodiments. The expression “the positions at which V¹ is bound to W-T-Z³-(CR⁶R⁷)ₙ— and to —Ar—CH₂—NR¹—B^{B31}-D-E are the 5-position and 3-position of V¹, respectively” may be used in place of the expression (5, 3).

In the case where V¹ is a divalent group obtained by removing two hydrogen atoms from [1,2,4]-thiadiazole, (5, 3) is preferred, and (3, 5) may be preferred in some embodiments. The expression “the positions at which V¹ is bound to W-T-Z³-(CR⁶R⁷)ₙ— and to —Ar—CH₂—NR¹—B^{B31}D-E are the 5-position and 3-position of V¹, respectively” may be used in place of the expression (5, 3)

Hereinafter, general formula (3B) will be described in detail.

In the general formula (3B), W, T, Z³, R⁶, R⁷, n, V, Ar, R¹, D and E have the same meanings as the aforementioned.

Hereinafter, general formula (4) and general formula (5) will be described in detail.

In the general formula (4) and general formula (5), W, T, Z³, R⁶, R⁷, n, V, Ar, R¹, B³¹ and R^{E} have the same meanings as the aforementioned.

Hereinafter, general formula (6) will be described in detail.

R^{1B} represents a hydrogen atom or a C1-C4 alkyl group, but among these, a hydrogen atom, a methyl group or an ethyl group is preferred; a hydrogen atom or a methyl group is more preferred; and a hydrogen atom is particularly preferred.

Ar^{B} represents a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine;

this group may be substituted with one to two X^{1B}s, and when the group is substituted with two X^{1B}s, X^{1B}s may be the same or different;

X^{1B} represents a fluorine atom, a chlorine atom, a bromine atom, a trifluoromethyl group, —OR^{X1B}, —SR^{X1B} or —R^{X1B}; and

R^{X1B} represents a hydrogen atom, a C1-C6 alkyl group, a C3-C6 cycloalkyl group or a phenyl group.

Ar^{B} is preferably a divalent group obtained by removing two hydrogen atoms from a compound selected from the group consisting of benzene, furan, thiophene and pyridine; more preferably a divalent group obtained by removing two hydrogen atoms from a compound selected from the group consisting of benzene and thiophene; and even more preferably a divalent group obtained by removing two hydrogen atoms from benzene. Furthermore, a divalent group obtained by removing two hydrogen atoms from thiophene may be more preferred in some embodiments. Moreover, a divalent group obtained by removing two hydrogen atoms from pyridine may be more preferred in some embodiments.

X^{1B} is preferably a fluorine atom, a chlorine atom, a bromine atom, —OR^{X1B} or —R^{X1B}; more preferably a fluorine atom, a bromine atom, —OR^{X1B} or —R^{X1B}; even more preferably —OR^{X1B} or —R^{X1B}; and particularly preferably —R^{X1B}. Furthermore, a fluorine atom may be particularly preferred in some embodiments. Moreover, a bromine atom may be particularly preferred in some embodiments.

R^{X1B} is preferably a hydrogen atom or a C1-C6 alkyl group; more preferably a hydrogen atom, a methyl group, an ethyl group, a propyl group or an isopropyl group; and even more preferably a methyl group or an ethyl group.

When the substitution is made with two or more X^{1B}s, X^{1B}s may be highly preferably a combination of the aforementioned preferred examples.

B^{B} may be exemplified by a C2 alkylene group, while the group may be substituted to a possible extent with one to four X^{BB}s, and when the group is substituted with two or more X^{BB}s, X^{BB}s may be the same or different;

X^{BB} represents a fluorine atom or a C1-C4 alkyl group which may be substituted with one to five G^{XBB}s, and when the alkyl group is substituted with two or more G^{XBB}s, G^{XBB}s may be the same or different; and

G^{XBB} represents a halogen atom, a hydroxyl group, or an amino group.

X^{BB} is preferably a fluorine atom, a methyl group or an ethyl group; more preferably a fluorine atom or a methyl group; and even more preferably a fluorine atom.

When the substitution is made with two or more X^{BB}s, X^{BB}s is highly preferably a combination of the aforementioned preferred examples.

G^{XBB} is preferably a fluorine atom, a hydroxyl group or an amino group; more preferably a fluorine atom or a hydroxyl group; and even more preferably a fluorine atom.

When the substitution is made with two or more G^{XBB}s, G^{XBB}S is highly preferably a combination of the a aforementioned preferred examples.

R^{EB} represents a hydrogen atom, a C1-C4 alkyl group, —(CH₂)_{mB}N(R^{EB1})(R^{EB2}) or —C(R^{EB3})₂OC(O)A^{EB}R^{EB4};

m^{B} denotes an integer of 2 or 3;

R^{EB1} and R^{EB2}, which may be the same or different, each independently represent a methyl group, an ethyl group or a propyl group, or R^{EB1} and R^{EB2} may be joined to form a 3- to 6-membered ring together with the nitrogen atom, thus representing a saturated nitrogen-containing cycloalkyl group, or to form a morpholino group together with the nitrogen atom;

R^{EB3} represents a hydrogen atom, a methyl group, an ethyl group or a propyl group;

R^{EB4} represents a C1-C4 alkyl group, a C3-C6 cycloalkyl group or a phenyl group; and

A^{EB} represents a single bond or an oxygen atom.

R^{EB} is preferably a hydrogen atom, a methyl group, an ethyl group or -(CH₂)_{mB}N(R^{EB1})(R^{EB2}); more preferably a hydrogen atom, a methyl group or an ethyl group; and particularly preferably a hydrogen atom. Examples of —(CH₂)_{mB}N(R^{EB1})(R^{EB2}) include a 2-(N,N-dimethylamino)ethyl group, a 2-(N,N-diethylamino)ethyl group, a 2-(N,N-dipropylamino)ethyl group, a 3-(N,N-dimethylamino)propyl group, a 3-(N,N-diethylamino)propyl group, a 2-(N,N-dipropylamino)propyl group, a 2-pyrrolidin-1-ylethyl group, a 2-piperidin-1-ylethyl group, a 2-morpholin-4-ylethyl group, a 3-pyrrolidin-1-ylpropyl group, a 3-piperidin-1-ylpropyl group, a 3-morpholin-4-ylpropyl group, and the like.

Examples of —C(R^{EB3})₂OC(O)A^{EB}R^{EB4} include an acetoxymethyl group, a propionyloxymethyl group, a butyryloxymethyl group, a (2-methylpropionyl)oxymethyl group, a (2,2-dimethylpropionyl)oxymethyl group, a cyclopropionyloxymethyl group, a cyclopentanoyloxymethyl group, a cyclohexanoyloxymethyl group, a phenylcarboxymethyl group, a 1-acetoxy-1-methylethyl group, a 1-methyl-1-(2-methylpropionyloxy)ethyl group, a 1-cyclopentanoyloxy-1-methylethyl group, a 1-cyclohexanoyloxy-1-methylethyl group, a methoxycarbonyloxymethyl group, an ethoxycarbonyloxymethyl group, an isopropyloxycarbonyloxymethyl group, a t-butyloxycarbonyloxymethyl group, a cyclopropyloxycarbonyloxymethyl group, a cyclopentyloxycarbonyloxymethyl group, a cyclohexyloxycarbonyloxymethyl group, a phenyloxycarbonyloxymethyl group, a 1-methoxycarbonyloxy-1-methylethyl group, a 1-ethoxycarbonyloxy-1-methylethyl group, a 1-isopropyloxycarbonyloxy-1-methylethyl group, a 1-t-butyloxycarbonyloxy-1-methylethyl group, a 1-cyclopropyloxycarbonyloxy-1-methylethyl group, a 1-cyclopentyloxycarbonyloxy-1-methylethyl group, a 1-cyclohexyloxycarbonyloxy-1-methylethyl group, a 1-methyl-1-phenyloxycarbonyloxyethyl group, and the like.

V^{1B} represents a single bond, or a divalent group obtained by removing two hydrogen atoms from a compound selected from the group consisting of oxadiazole and thiadiazole, but among these, a divalent group obtained by removing two hydrogen atoms from a compound selected from the group consisting of [1,2,4]-oxadiazole, [1,3,4]-oxadiazole, [1,2,4]-thiadiazole and [1,3,4]-thiadiazole is preferred; a divalent group obtained by removing two hydrogen atoms from a compound selected from the group consisting of [1,2,4]-oxadiazole and [1,3,4]-thiadiazole is more preferred; and a divalent group obtained by removing two hydrogen atoms from [1,2,4]-oxadiazole is even more preferred.

When V^{1B} is a divalent group obtained by removing two hydrogen atoms from a compound selected from the group consisting of oxadiazole and thiadiazole, preferred examples of the positions at which V^{1B} is bound to W^{B}-T^{B}-Z^{3B}-(CR^{6B}R^{7B})ₙ^{B}- and to —Ar^{B}-CH₂—NR^{1B}—B^{B}—CO₂R^{EB} are presented in the following as (binding position for W^{B}-T^{B}-Z^{3B}-(CR^{6B}R^{7B})ₙ^{B}-, binding position for —Ar^{B}-CH₂—NR^{1B}—CO₂R^{EB}).

When V^{1B} is a divalent group obtained by removing two hydrogen atoms from [1,2,4]-oxadiazole, (5,3) is preferred, and (3, 5) may be preferred in some embodiments. The expression “the positions at which V^{1B} is bound to W^{B}-T^{B}-Z^{3B}-(CR^{6B}R^{7B})ₙ^{B}- and to —Ar^{B}-CH₂—NR^{1B}—B^{B}—CO₂R^{EB} are the 5-position and 3-position of V^{1B}, respectively” may be used in place of the expression (5, 3).

When V^{1B} is a divalent group obtained by removing two hydrogen atoms from [1,2,4]-thiadiazole, (5, 3) is preferred, and (3, 5) may be preferred in some embodiments. The expression “the positions at which V¹⁸ is bound to W^{B}-T^{B}-Z^{3B}-(CR^{6B}R^{7B})ₙ^{B}- and to —Ar^{1B}-CH₂—NR^{1B}—B^{B}—CO₂R^{EB} are the 5-position and 3-position of V^{1B}, respectively” may be used in place of the expression (5, 3).

W^{B} represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{WB}R^{WB2}, a C3-C7 cycloalkoxy group which may be substituted with NR^{WB}R^{WB2}, or a C1-C6 alkoxy group which may be substituted with a C3-C7 cycloalkyl group in which one to two carbon atoms in the ring may be substituted with a nitrogen atom(s), or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring may be substituted with one to two X^{4B}s, and when the monovalent group is substituted with two X^{4B}s, X^{4B}s may be the same or different;

X^{4B} represents a fluorine atom, a chlorine atom, a trifluoromethyl group, a trifluoromethoxy group, a cyano group, —OR^{X4B}, —SR^{X4B}, or —R^{X4B};

R^{X4B} represents a hydrogen atom, a C1-C6 alkyl group, a C3-C6 cycloalkyl group, or a phenyl group;

R^{WB} and R^{WB2}, which may be the same or different, each independently represent a hydrogen atom or a C1-C6 alkyl group.

X^{4B} is preferably a fluorine atom, a trifluoromethyl group, a trifluoromethoxy group, a cyano group, —OR^{X4B l or —R}^{X4B}; more preferably a fluorine atom, a trifluoromethyl group or a cyano group; and even more preferably a fluorine atom or a cyano group. Furthermore, —OR^{X4B} or —R^{X4B} may be more preferred in some embodiments. Moreover, a trifluoromethyl group may be more preferred in some embodiments. Apart from these, X^{4B} may be preferably a trifluoromethoxy group, a cyano group, —OR^{X4B} or —R^{X4B}; and more preferably a trifluoromethoxy group or a cyano group in some embodiments. Furthermore, a cyano group or —OR^{X4B} may be preferred in some embodiments.

R^{X4B} is preferably a hydrogen atom, a methyl group, an ethyl group, a propyl group, an isopropyl group, a cyclopentyl group, a cyclohexyl group or a phenyl group; more preferably a methyl group, an ethyl group, a propyl group or an isopropyl group; even more preferably a methyl group or an ethyl group; and particularly preferably a methyl group.

W^{B} is preferably a hydrogen atom, a C1-C6 alkyl group which may substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene and pyridine; more preferably a C1-C6 alkyl group which may be substituted with 1 to 7 fluorine atoms, or a C3-C7 cycloalkyl group which may be substituted with 1 to 7 fluorine atoms; and even more preferably a C5-C6 alkyl group which may be substituted with 1 to 7 fluorine atoms, or a C5-C7 cycloalkyl group which may be substituted with 1 to 7 fluorine atoms. Furthermore, a monovalent group obtained by removing one hydrogen atom from a compound selected from the group consisting of benzene and thiophene may be more preferred in some embodiments. Moreover, a monovalent group obtained by removing one hydrogen atom from pyridine may be more preferred in some embodiments. In addition to these, a monovalent group obtained by removing one hydrogen atom from benzene may be more preferred in some embodiments.

Furthermore, a monovalent group obtained by removing one hydrogen atom from a compound selected from the group consisting of benzene, naphthalene, furan, thiophene and pyridine, which monovalent group is substituted with the above-described preferred X^{4B} or R^{X4B}, is preferred; a monovalent group obtained by removing one hydrogen atom from a compound selected from the group consisting of benzene, thiophene and pyridine, which monovalent group is substituted with the above-described preferred X^{4B} or R^{X4B}, is more preferred; and a monovalent group obtained by removing one hydrogen atom from a compound selected from the group consisting of benzene and thiophene, which monovalent group is substituted with the above-described preferred X^{4B} or R^{X4B}, is more preferred in some embodiments.

T^{B} represents a single bond, a C1-C6 alkylene group, —O—, —OCH₂—, —NR^{TB}-, —NR^{TB}NHCO— or —CONR^{TB}-; and

R^{TB} represents a hydrogen atom or a C1-C6 alkyl group.

T^{B} is preferably a single bond, a C1-C6 alkylene group, —O—, —OCH₂— or —CONR^{TB}-; more preferably a single bond, a methylene group, an ethylene group or —O—; even more preferably a single bond or —O—; and particularly preferably a single bond. Furthermore, —CONR^{TB} is more preferred in some embodiments.

Z^{3B} represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine and imidazo(1,2-a)pyridine;

with the proviso that Z^{3B} may be further substituted with one to four X^{Z3B}s, and when Z^{3B} is substituted with two or more X^{Z3B}s, X^{Z3B}s may be the same or different;

X^{Z3B} represents a fluorine atom, a chlorine atom, a trifluoromethyl group, a cyano group, —OR^{XZ3B}, —SR^{XZ3B} or —R^{XZ3B};

R^{XZ3B} represents a hydrogen atom, a C1-C6 alkyl group, a C3-C6 cycloalkyl group or a phenyl group (provided that the C1-C6 alkyl group, the C3-C6 cycloalkyl group or the phenyl group may be respectively substituted with a fluorine atom).

Z^{3B} is preferably a C5-C6 cycloalkylene group, or a divalent group obtained by removing two hydrogen atoms from a compound selected from the group consisting of benzene, furan, thiophene, oxazole, isoxazole, oxadiazole, thiazole, isothiazole, thiadiazole, imidazole, pyrazole, pyran, pyridine, pyridazine, pyrimidine, pyrazine, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline and isoquinoline; more preferably a C5-C6 cycloalkylene group, or a divalent group obtained by removing two hydrogen atoms from a compound selected from the group consisting of benzene, furan, thiophene, oxazole, isoxazole, oxadiazole, thiazole, isothiazole, thiadiazole, imidazole, pyrazole, pyran, pyridine, pyridazine, pyrimidine and pyrazine; even more preferably a C5-C6 cycloalkylene group, or a divalent group obtained by removing two hydrogen atoms from a compound selected from the group consisting of benzene, thiophene and pyridine; still more preferably a divalent group obtained by removing two hydrogen atoms from a compound selected from the group consisting of benzene and thiophene; and most preferably a divalent group obtained by removing two hydrogen atoms from benzene. Furthermore, a divalent group obtained by removing two hydrogen atoms from pyridine is most preferred in some embodiments. Also, the preferred examples of Z^{3B} which are each independently substituted with one or two of X^{Z3B} are also highly preferred.

X^{Z3B} is preferably a fluorine atom, a chlorine atom, a trifluoromethyl group, a trifluoromethoxy group, a cyano group, —OR^{XZ3B} or —R^{XZ3B}; more preferably a fluorine atom, a trifluoromethoxy group, a cyano group or —R^{XZ3B}; even more preferably a fluorine atom, a cyano group or —R^{XZ3B}; and particularly preferably a fluorine atom or a cyano group. Furthermore, a trifluoromethoxy group is particularly preferred in some embodiments. Moreover, —R^{XZ3B} is particularly preferred in some embodiments.

R^{XZ3B} is preferably a hydrogen atom or a C1-C6 alkyl group; more preferably a methyl group, an ethyl group or a propyl group; even more preferably a methyl group or an ethyl group; and particularly preferably a methyl group. Furthermore, a methyl group substituted with a fluorine atom is particularly preferred in some embodiments.

n^{B} is preferably 0 or 1; and more preferably 0.

For the compound represented by general formula (2), the respective combinations of substituents are not particularly limited, but for example:

<A1> a compound wherein Ar is a divalent group obtained by removing two hydrogen atoms from naphthalene which may be substituted with one to two of a fluorine atom, a chlorine atom, a bromine atom, a hydroxyl group, a methoxy group, a methyl group or an ethyl group;

<A2> a compound wherein Ar is a divalent group obtained by removing two hydrogen atoms from benzothiophene which may be substituted with one to two of a fluorine atom, a chlorine atom, a bromine atom, a hydroxyl group, a methoxy group, a methyl group or an ethyl group;

<A3> a compound wherein Ar is a divalent group obtained by removing two hydrogen atoms from quinoline which may be substituted with one to two of a fluorine atom, a chlorine atom, a bromine atom, a hydroxyl group, a methoxy group, a methyl group or an ethyl group;

<A4> a compound wherein Ar is a divalent group obtained by removing two hydrogen atoms from benzene which may be substituted with one to two of a fluorine atom, a chlorine atom, a bromine atom, a hydroxyl group, a methoxy group, a methyl group or an ethyl group;

<A5> a compound wherein Ar is a divalent group obtained by removing two hydrogen atoms from isoquinoline which may be substituted with one to two of a fluorine atom, a chlorine atom, a bromine atom, a hydroxyl group, a methoxy group, a methyl group or an ethyl group;

<A6> a compound wherein Ar is a divalent group obtained by removing two hydrogen atoms from indazole which may be substituted with one to two of a fluorine atom, a chlorine atom, a bromine atom, a hydroxyl group, a methoxy group, a methyl group or an ethyl group;

<A7> a compound wherein Ar is a divalent group obtained by removing two hydrogen atoms from pyridine which may be substituted with one to two of a fluorine atom, a chlorine atom, a bromine atom, a hydroxyl group, a methoxy group, a methyl group or an ethyl group;

<A8> a compound wherein Ar is a divalent group obtained by removing two hydrogen atoms from furan which may be substituted with one to two of a fluorine atom, a chlorine atom, a bromine atom, a hydroxyl group, a methoxy group, a methyl group or an ethyl group;

<A9> a compound wherein Ar is a divalent group obtained by removing two hydrogen atoms from thiophene which may be substituted with one to two of a fluorine atom, a chlorine atom, a bromine atom, a hydroxyl group, a methoxy group, a methyl group or an ethyl group;

<A10> a compound wherein Ar is a divalent group obtained by removing two hydrogen atoms from naphthalene which may be substituted with one to two of a chlorine atom, a bromine atom, a methoxy group, a methyl group or an ethyl group;

<A11> a compound wherein Ar is a divalent group obtained by removing two hydrogen atoms from benzothiophene which may be substituted with one to two of a chlorine atom, a bromine atom, a methoxy group, a methyl group or an ethyl group;

<A12> a compound wherein Ar is a divalent group obtained by removing two hydrogen atoms from quinoline which may be substituted with one to two of a chlorine atom, a bromine atom, a methoxy group, a methyl group or an ethyl group;

<A13> a compound wherein Ar is a divalent group obtained by removing two hydrogen atoms from benzene which may be substituted with one to two of a chlorine atom, a bromine atom, a methoxy group, a methyl group or an ethyl group;

<A14> a compound wherein Ar is a divalent group obtained by removing two hydrogen atoms from pyridine which may be substituted with one to two of a chlorine atom, a bromine atom, a methoxy group, a methyl group or an ethyl group;

<A15> a compound wherein Ar is a divalent group obtained by removing two hydrogen atoms from furan which may be substituted with one to two of a chlorine atom, a bromine atom, a methoxy group, a methyl group or an ethyl group;

<A16> a compound wherein Ar is a divalent group obtained by removing two hydrogen atoms from thiophene which may be substituted with one to two of a chlorine atom, a bromine atom, a methoxy group, a methyl group or an ethyl group;

<A17> a compound wherein Ar is a divalent group obtained by removing two hydrogen atoms from naphthalene, benzothiophene, quinoline, benzene, isoquinoline, indazole, pyridine, furan or thiophene, with the proviso that the compound may be substituted with one to two of a fluorine atom, a chlorine atom, a bromine atom, a hydroxyl group, a methoxy group, a methyl group or an ethyl group;

<A18> a compound wherein Ar is a divalent group obtained by removing two hydrogen atoms from naphthalene, benzothiophene, quinoline, benzene, pyridine, furan or thiophene, with the proviso that the compound may be substituted with one to two of a chlorine atom, a bromine atom, a methoxy group, a methyl group or an ethyl group;

<A19> the compound according to <A1> or <A10>, wherein the positions at which the divalent group obtained by removing two hydrogen atoms from naphthalene is bound to Y and to —(CR²R³)m¹—NR¹—(CR⁴R⁵)ₘ²—B-D-E are (1, 4) or (2, 6);

<A20> the compound according to <A2> or <A11>, wherein the positions at which the divalent group obtained by removing two hydrogen atoms from benzothiophene is bound to Y and to —(CR²R³)m¹—NR¹—(CR⁴R⁵)m²—B-D-E are (2, 6) or (6, 2);

<A21> the compound according to <A3> or <A12>, wherein the positions at which the divalent group obtained by removing two hydrogen atoms from quinoline is bound to Y and to —(CR²R³)m¹—NR¹—(CR⁴R⁵)m²—B-D-E are (2, 6), (3, 7), (6, 2), or (7, 3);

<A22> the compound according to <A4> or <A13>, wherein the positions at which the divalent group obtained by removing two hydrogen atoms from benzene is bound to Y and to —(CR²R³)m¹—NR¹—(CR⁴R⁵)m²—B-D-E are (1, 3) or (1, 4);

<A23> the compound according to <A4> or <A13>, wherein the positions at which the divalent group obtained by removing two hydrogen atoms from benzene is bound to Y and to —(CR²R³)m¹—NR¹—(CR⁴R⁵)m²—B-D-E are (1, 4);

<A24> the compound according to <A5>, wherein the positions at which the divalent group obtained by removing two hydrogen atoms from isoquinoline is bound to Y and to —(CR²R³)m¹—NR¹—(CR⁴R⁵)m²—B-D-E are (1, 4), (1, 5), (3, 7), (4, 1), (5, 1) or (7, 3);

<A25> the compound according to <A6>, wherein the positions at which the divalent group obtained by removing two hydrogen atoms from indazole is bound to Y and to —(CR²R³)m¹—NR¹—(CR⁴R⁵)m²—B-D-E are (1, 3), (1, 5), (2, 6), (3, 1), (5, 1) or (6, 2);

<A26> the compound according to <A7> or <A14>, wherein the positions at which the divalent group obtained by removing two hydrogen atoms from pyridine is bound to Y and to —(CR²R³)ₘ¹—NR¹—(CR⁴R⁵)m²—B-D-E are (2, 5), (2, 6) or (3, 6);

<A27> the compound according to <A9> or <A16>, wherein the positions at which the divalent group obtained by removing two hydrogen atoms from furan is bound to Y and to —(CR²R³)m¹NR¹—(CR⁴R⁵)m²—B-D-E are (2, 5);

<A28> the compound according to <A9> or <A16>, wherein the positions at which the divalent group obtained by removing two hydrogen atoms from thiophene is bound to Y and to —(CR²R³) m¹—NR¹—(CR⁴R⁵)m²—B-D-E are (2, 5);

<B1> a compound wherein B is B¹;

<B2> a compound wherein B is a divalent group obtained by removing two hydrogen atoms from a 3- to 7-membered saturated ring compound composed of carbon atoms;

<B3> a compound wherein B is a divalent group obtained by removing two hydrogen atoms from a 3- to 7-membered partially unsaturated ring compound composed of carbon atoms;

<B4> a compound wherein B is a divalent group obtained by removing two hydrogen atoms from a 3- to 6-membered saturated ring compound composed of carbon atoms;

<B5> a compound wherein B is a divalent group obtained by removing two hydrogen atoms from a 4- to 5-membered saturated ring compound composed of carbon atoms;

<B6> a compound wherein B is a divalent group obtained by removing two hydrogen atoms from a 4-membered saturated ring compound composed of carbon atoms;

<B7> a compound wherein B is a divalent group obtained by removing two hydrogen atoms from a 5-membered saturated ring compound composed of carbon atoms;

<B8> a compound wherein B is a divalent group obtained by removing two hydrogen atoms from a 5-membered partially unsaturated ring compound composed of carbon atoms;

<B9> a compound wherein B is a divalent group obtained by removing two hydrogen atoms from a 3- to 6-membered saturated ring compound composed of carbon atoms, which compound is substituted with one to two of X²;

<B10> a compound wherein B is a divalent group obtained by removing two hydrogen atoms from a 4- to 5-membered saturated ring compound composed of carbon atoms, which compound is substituted with one to two of X²;

<B11> a compound wherein B is a divalent group obtained by removing two hydrogen atoms from a 4-membered saturated ring compound composed of carbon atoms, which compound is substituted with one to two of X²;

<B12> a compound wherein B is a divalent group obtained by removing two hydrogen atoms from a 5-membered saturated ring compound composed of carbon atoms, which compound is substituted with one to two of X²;

<B13> the compound according to <B6> or <B11>, wherein B is a divalent group obtained by removing two hydrogen atoms from a 4-membered saturated ring compound composed of carbon atoms, and the positions at which the divalent group is bound to —NR¹— and to -D-E are (1, 3);

<B14> the compound according to <B7> or <B12>, wherein B is a divalent group obtained by removing two hydrogen atoms from a 5-membered saturated ring compound composed of carbon atoms, and the positions at which the divalent group is bound to —NR¹- and to -D-E are (1, 3);

<B15> the compound according to <B6>, <B11> or <B13>, wherein the relationship between the bond between B and —NR¹—, and the bond between B and -D-E is a cis relationship;

<B16> the compound according to <B6>, <B11 > or <B13>, wherein the relationship between B and —NR¹—, and the bond between B and -D-E is a trans relationship;

<B17> the compound according to <B7>, <B12> or <B14>, wherein the relationship between B and —NR¹—, and the bond between B and -D-E is a cis relationship;

<B18> the compound according to <B7>, <B12> or <B14>, wherein the relationship between B and —NR¹—, and the bond between B and -D-E is a trans relationship;

<C1> a compound which is <B1> in conjunction with any one of <A1> to <A28>;

<C2> a compound which is <B2> in conjunction with any one of <A1> to <A28>;

<C3> a compound which is <B3> in conjunction with any one of <A1> to <A28>;

<C4> a compound which is <B4> in conjunction with any one of <A1> to <A28>;

<C5> a compound which is <B5> in conjunction with any one of <A1> to <A28>;

<C6> a compound which is <B6> in conjunction with any one of <A1> to <A28>;

<C7> a compound which is <B7> in conjunction with any one of <A1> to <A28>;

<C8> a compound which is <B8> in conjunction with any one of <A1> to <A28>;

<C9> a compound which is <B9> in conjunction with any one of <A1> to <A28>;

<C10> a compound which is <B10> in conjunction with any one of <A1> to <A28>;

<C11 > a compound which is <B11> in conjunction with any one of <A1> to <A28>;

<C12> a compound which is <B12> in conjunction with any one of <A1> to <A28>;

<C13> a compound which is <B13> in conjunction with any one of <A1> to <A28>;

<C14> a compound which is <B14> in conjunction with any one of <A1> to <A28>;

<C15> a compound which is <B15> in conjunction with any one of <A1> to <A28>;

<C16> a compound which is <B16> in conjunction with any one of <A1> to <A28>;

<C17> a compound which is <B17> in conjunction with any one of <A1> to <A28>;

<C18> a compound which is <B18> in conjunction with any one of <A1> to <A28>;

<D1> a compound wherein E is a hydroxyl group;

<D2> a compound wherein E is —CO₂RE;

<D3> a compound wherein E is —CO₂H;

<D4> a compound wherein E is —CO₂Me;

<D5> a compound wherein E is —CO₂Et;

<D6> a compound wherein E is a 1H-tetrazol-5-yl group;

<E1> a compound which is <D1> in conjunction with any one of <A1> to <C18>;

<E2> a compound which is <D2> in conjunction with any one of <A1> to <C1B>;

<E3> a compound which is <D3> in conjunction with any one of <A1> to <C18>;

<E4> a compound which is <D4> in conjunction with any one of <A1> to <C18>;

<E5> a compound which is <D5> in conjunction with any one of <A1> to <C18>;

<E6> a compound which is <D6> in conjunction with any one of <A1> to <C18>;

<F1> a compound wherein Z in Y is a C3-C6 cycloalkylene group;

<F2> a compound wherein Z in Y is a C5-C6 cycloalkylene group;

<F3> a compound wherein Z in Y is a divalent group obtained by removing two hydrogen atoms from benzene;

<F4> a compound wherein Z in Y is a divalent group obtained by removing two hydrogen atoms from a monocyclic aromatic heterocyclic compound;

<F5> a compound wherein Z in Y is a divalent group obtained by removing two hydrogen atoms from a bicyclic aromatic hydrocarbon ring compound;

<F6> a compound wherein Z in Y is a divalent group obtained by removing two hydrogen atoms from a bicyclic aromatic heterocyclic compound;

<F7> a compound wherein Z in Y is a divalent group obtained by removing two hydrogen atoms from furan;

<F8> a compound wherein Z in Y is a divalent group obtained by removing two hydrogen atoms from thiophene;

<F9> a compound wherein Z in Y is a divalent group obtained by removing two hydrogen atoms from oxazole;

<F10> a compound wherein Z in Y is a divalent group obtained by removing two hydrogen atoms from isoxazole;

<F11 > a compound wherein Z in Y is a divalent group obtained by removing two hydrogen atoms from oxadiazole;

<F12> a compound wherein Z in Y is a divalent group obtained by removing two hydrogen atoms from thiazole;

<F13> a compound wherein Z in Y is a divalent group obtained by removing two hydrogen atoms from isothiazole;

<F14> a compound wherein Z in Y is a divalent group obtained by removing two hydrogen atoms from thiadiazole;

<F15> a compound wherein Z in Y is a divalent group obtained by removing two hydrogen atoms from imidazole;

<F16> a compound wherein Z in Y is a divalent group obtained by removing two hydrogen atoms from pyrazole;

<P17> a compound wherein Z in Y is a divalent group obtained by removing two hydrogen atoms from pyran;

<F18> a compound wherein Z in Y is a divalent group obtained by removing two hydrogen atoms from pyridine;

<P19> a compound wherein Z in Y is a divalent group obtained by removing two hydrogen atoms from pyridazine;

<F20> a compound wherein Z in Y is a divalent group obtained by removing two hydrogen atoms from pyrimidine;

<F21> a compound wherein Z in Y is a divalent group obtained by removing two hydrogen atoms from pyrazine;

<F22> a compound wherein Z in Y is a divalent group obtained by removing two hydrogen atoms from naphthalene;

<F23> a compound wherein Z in Y is a divalent group obtained by removing two hydrogen atoms from benzofuran;

<F24> a compound wherein Z in Y is a divalent group obtained by removing two hydrogen atoms from benzo[b]thiophene;

<F25> a compound wherein Z in Y is a divalent group obtained by removing two hydrogen atoms from indole;

<F26> a compound wherein Z in Y is a divalent group obtained by removing two hydrogen atoms from isoindole;

<F27> a compound wherein Z in Y is a divalent group obtained by removing two hydrogen atoms from indolizine;

<F28> a compound wherein Z in Y is a divalent group obtained by removing two hydrogen atoms from 1H-indazole;

<F29> a compound wherein Z in Y is a divalent group obtained by removing two hydrogen atoms from 2H-indazole;

<F30> a compound wherein Z in Y is a divalent group obtained by removing two hydrogen atoms from 1H-benzimidazole;

<F31> a compound wherein Z in Y is a divalent group obtained by removing two hydrogen atoms from benzoxazole;

<F32> a compound wherein Z in Y is a divalent group obtained by removing two hydrogen atoms from benzo[d]isoxazole;

<F33> a compound wherein Z in Y is a divalent group obtained by removing two hydrogen atoms from benzo[c]isoxazole;

<F34> a compound wherein Z in Y is a divalent group obtained by removing two hydrogen atoms from benzothiazole;

<F35> a compound wherein Z in Y is a divalent group obtained by removing two hydrogen atoms from benzo[d]isothiazole;

<F36> a compound wherein Z in Y is a divalent group obtained by removing two hydrogen atoms from benzo[c]isothiazole;

<F37> a compound wherein Z in Y is a divalent group obtained by removing two hydrogen atoms from 1H-benzotriazole;

<F38> a compound wherein Z in Y is a divalent group obtained by removing two hydrogen atoms from benzo[1,2,5]thiadiazole;

<F39> a compound wherein Z in Y is a divalent group obtained by removing two hydrogen atoms from quinoline;

<F40> a compound wherein Z in Y is a divalent group obtained by removing two hydrogen atoms from isoquinoline;

<F41> the compound according to <F1> to <F40>, wherein Z is substituted with one to two substituent X⁵;

<F42> the compound according to <F41>, wherein Z is substituted with one substituent X⁵;

<F43> the compound according to <F41> or <F42>, wherein X⁵ is a fluorine atom, a trifluoromethyl group, a cyano group, —OR^{X5}, or R^{X5};

<F44> the compound according to <F41> or <F42>, wherein X⁵ is a trifluoromethyl group, or —R^{X5};

<F45> the compound according to <F43> or <F44>, wherein R^{X5} is a hydrogen atom, a methyl group, an ethyl group, a propyl group, an isopropyl group, a cyclopentyl group, a cyclohexyl group or a phenyl group;

<F46> the compound according to <F41> or <F42>, wherein X⁵ is a trifluoromethyl group;

<G1> a compound which is <F1> in conjunction with any one of <A1> to <E6>;

<G2> a compound which is <F2> in conjunction with any one of <A1> to <E6>;

<G3> a compound which is <F3> in conjunction with any one of <A1> to <E6>;

<G4> a compound which is <F4> in conjunction with any one of <A1> to <E6>;

<G5> a compound which is <F5> in conjunction with any one of <A1> to <E6>;

<G6> a compound which is <F6> in conjunction with any one of <A1> to <E6>;

<G7> a compound which is <F7> in conjunction with any one of <A1> to <E6>;

<G8> a compound which is <F8> in conjunction with any one of <A1> to <E6>;

<G9> a compound which is <F9> in conjunction with any one of <A1> to <E6>;

<G10> a compound which is <F10> in conjunction with any one of <A1> to <E6>;

<G11> a compound which is <F11 > in conjunction with any one of <A1> to <E6>;

<G12> a compound which is <F12> in conjunction with any one of <A1> to <E6>;

<G13> a compound which is <F13> in conjunction with any one of <A1> to <E6>;

<G14> a compound which is <F14> in conjunction with any one of <A1> to <E6>;

<G15> a compound which is <F15> in conjunction with any one of <A1> to <E6>;

<G16> a compound which is <F16> in conjunction with any one of <A1> to <E6>;

<G17> a compound which is <F17> in conjunction with any one of <A1> to <E6>;

<G18> a compound which is <F18> in conjunction with any one of <A1> to <E6>;

<G19> a compound which is <F19> in conjunction with any one of <A1> to <E6>;

<G20> a compound which is <F20> in conjunction with any one of <A1> to <E6>;

<G21> a compound which is <F21> in conjunction with any one of <A1> to <E6>;

<G22> a compound which is <F22> in conjunction with any one of <A1> to <E6>;

<G23> a compound which is <F23> in conjunction with any one of <A1> to <E6>;

<G24> a compound which is <F24> in conjunction with any one of <A1> to <E6>;

<G25> a compound which is <F25> in conjunction with any one of <A1> to <E6>;

<G26> a compound which is <F26> in conjunction with any one of <A1> to <E6>;

<G27> a compound which is <F27> in conjunction with any one of <A1> to <E6>;

<G28> a compound which is <F28> in conjunction with any one of <A1> to <E6>;

<G29> a compound which is <F29> in conjunction with any one of <A1> to <E6>;

<G30> a compound which is <F30> in conjunction with any one of <A1> to <E6>;

<G31> a compound which is <F31> in conjunction with any one of <A1> to <E6>;

<G32> a compound which is <F32> in conjunction with any one of <A1> to <E6>;

<G33> a compound which is <F33> in conjunction with any one of <A1> to <E6>;

<G34> a compound which is <F34> in conjunction with any one of <A1> to <E6>;

<G35> a compound which is <F35> in conjunction with any one of <A1> to <E6>;

<G36> a compound which is <F36> in conjunction with any one of <A1> to <E6>;

<G37> a compound which is <F37> in conjunction with any one of <A1> to <E6>;

<G38> a compound which is <F38> in conjunction with any one of <A1> to <E6>;

<G39> a compound which is <F39> in conjunction with any one of <A1> to <E6>;

<G40> a compound which is <F40> in conjunction with any one of <A1> to <E6>;

<G41> a compound which is <F41> in conjunction with any one of <A1> to <E6>;

<G42> a compound which is <F42> in conjunction with any one of <A1> to <E6>;

<G43> a compound which is <F43> in conjunction with any one of <A1> to <E6>;

<G44> a compound which is <F44> in conjunction with any one of <A1> to <E6>;

<G45> a compound which is <F45> in conjunction with any one of <A1> to <E6>;

<G46> a compound which is <F46> in conjunction with any one of <A1> to <E6>;

<H1> a compound wherein W is a hydrogen atom;

<H2> a compound wherein W is a C1-C6 alkyl group which may be substituted with 1 to 7 fluorine atoms;

<H3> a compound wherein W is a C3-C7 cycloalkyl group which may be substituted with 1 to 7 fluorine atoms;

<H4> a compound wherein W is a monovalent group obtained by removing one hydrogen atom from benzene which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H5> a compound wherein W is a monovalent group obtained by removing one hydrogen atom from naphthalene which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H6> a compound wherein W is a monovalent group obtained by removing one hydrogen atom from thiophene which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H7> a compound wherein W is a monovalent group obtained by removing one hydrogen atom from benzothiophene which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H8> a compound wherein W is a monovalent group obtained by removing one hydrogen atom from benzofuran which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H9> a compound wherein W is a monovalent group obtained by removing one hydrogen atom from quinoline which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<R10 > a compound wherein W is a monovalent group obtained by removing one hydrogen atom from tetrahydroquinoline which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H11> a compound wherein W is a monovalent group obtained by removing one hydrogen atom from isoquinoline which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H12> a compound wherein W is a monovalent group obtained by removing one hydrogen atom from tetrahydroisoquinoline which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H13> a compound wherein W is a monovalent group obtained by removing one hydrogen atom from pyridine which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H14> a compound wherein W is a monovalent group obtained by removing one hydrogen atom from indole which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H15> a compound wherein W is a monovalent group obtained by removing one hydrogen atom from indazole which may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<H16> a compound wherein W is a monovalent group obtained by removing one hydrogen atom from a compound selected from the group consisting of a monocyclic aromatic heterocyclic compound, a bicyclic aromatic hydrocarbon ring compound, a bicyclic aromatic heterocyclic compound, and a bicyclic compound obtained by fusing a saturated heterocyclic ring with a monocyclic aromatic hydrocarbon ring, with the proviso that this group may be substituted with one to two of a fluorine atom, a chlorine atom, a trifluoromethyl group, a methoxy group, an ethoxy group, a propoxy group, an isopropoxy group, a cyclopentyloxy group, a cyclohexyloxy group or a phenoxy group;

<I1> a compound which is <H1> in conjunction with any one of <A1> to <G46>;

<I2> a compound which is <H2> in conjunction with any one of <A1> to <G46>;

<I3> a compound which is <H3> in conjunction with any one of <A1> to <G46>;

<I4> a compound which is <H4> in conjunction with any one of <A1> to <G46>;

<I5> a compound which is <H5> in conjunction with any one of <A1> to <G46>;

<I6> a compound which is <H6> in conjunction with any one of <A1> to <G46>;

<I7> a compound which is <H7> in conjunction with any one of <A1> to <G46>;

<I8> a compound which is <H8> in conjunction with any one of <A1> to <G46>;

<I9> a compound which is <H9> in conjunction with any one of <A1> to <G46>;

<I10> a compound which is <H10> in conjunction with any one of <A1> to <G46>;

<I11> a compound which is <H11> in conjunction with any one of <A1> to <G46>;

<I12> a compound which is <H12> in conjunction with any one of <A1> to <G46>;

<I13> a compound which is <H₁₃> in conjunction with any one of <A1> to <G46>;

<I14> a compound which is <H14> in conjunction with any one of <A1> to <G46>;

<I15> a compound which is <H15> in conjunction with any one of <A1> to <G46>;

<I16> a compound which is <H16> in conjunction with any one of <A1> to <G46>;

<J1> a compound wherein V is a single bond;

<J2> a compound wherein V is —O—;

<J3> a compound wherein V is —CO—;

<J4> a compound wherein V is —CR^{V}(OCR^{V2})CR^{V3}CR^{V4}-;

<J5> a compound wherein V is —CR^{V}═CR^{V2}—;

<J6> a compound wherein V is —C≡C—;

<J7> a compound wherein V is —CONR^{V}—;

<J8> a compound wherein V is a divalent group obtained by removing two hydrogen atoms from oxadiazole;

<J9> a compound wherein V is a divalent group obtained by removing two hydrogen atoms from thiadiazole;

<J10> the compound according to <J4>, <J5> or <J7>, wherein R^{V}, R^{V2}, R^{V3} and R^{V4} are, if present, each independently a hydrogen atom, a methyl group, or an ethyl group;

<J11 > the compound according to <J4>, <J5> or <J7>, wherein R^{V}, R^{V2}, R^{V3} and R^{V4} are, if present, each independently a hydrogen atom, or a methyl group;

<J12> the compound according to <J4>, <J5> or <J7>, wherein R^{V}, R^{V2}, R^{V3} and R^{V4} are, if present, respectively a hydrogen atom;

<K1> a compound which is <J1> in conjunction with any one of <A1> to <116>;

<K2> a compound which is <J2> in conjunction with any one of <A1> to <I16>;

<K3> a compound which is <J3> in conjunction with any one of <A1> to <I16>;

<K4> a compound which is <J4> in conjunction with any one of <A1> to <I16>;

<K5> a compound which is <J5> in conjunction with any one of <A1> to <I16>;

<K6> a compound which is <J6> in conjunction with any one of <A1> to <I16>;

<K7> a compound which is <J7> in conjunction with any one of <A1> to <I16>;

<K8> a compound which is <J8> in conjunction with any one of <A1> to <I16>;

<K9> a compound which is <J9> in conjunction with any one of <A1> to <I16>;

<K10> a compound which is <J10> in conjunction with any one of <A1> to <I16>;

<K11 > a compound which is <J11> in conjunction with any one of <A1> to <116>;

<K12> a compound which is <J12> in conjunction with any one of <A1> to <I16>;

<L1> a compound wherein X⁵ in Z is a fluorine atom;

<L2> a compound wherein X⁵ in Z is a chlorine atom;

<L3> a compound wherein X⁵ in Z is —OR^{X5};

<L4> a compound wherein X⁵ in Z is —R^{X5};

<L5> a compound wherein X⁵ in Z is a cyano group;

<M1> a compound which is <L1> in conjunction with any one of <A1> to <K12> (provided that <F44> to <F46> are excluded);

<M2> a compound which is <L2> in conjunction with any one of <A1> to <K12> (provided that <F43> to <F46> are excluded);

<M3> a compound which is <L3> in conjunction with any one of <A1> to <K12> (provided that <F44> to <F46> are excluded);

<M4> a compound which is <L4> in conjunction with any one of <A1> to <K12> (provided that <F44> to <F46> are excluded);

<M5> a compound which is <L5> in conjunction with any one of <A1> to <K12> (provided that <F44> to <F46> are excluded);

<N1> a compound wherein R¹ to R⁷ are hydrogen atoms;

<O1> a compound which is <N1> in conjunction with any one of <A1> to <M5>;

<P1> a compound wherein m¹=1;

<Q1> a compound which is <P1> in conjunction with any one of <A1> to <O1>;

<R1 > a compound wherein m²=0;

<S1> a compound which is <R1 > in conjunction with any one of <A1> to <Q1>;

<T1> a compound wherein D is a single bond;

<U1> a compound which is <T1> in conjunction with any one of <A1> to <S1>;

<V1> a compound wherein T is a single bond;

<V2> a compound wherein T is a methylene group;

<V3> a compound wherein T is an ethylene group;

<V4> a compound wherein T is —O—;

<V5> a compound wherein T is —CONR^{T}—;

<W1> a compound which is <V1> in conjunction with any one of <A1> to <U1> above;

<W2> a compound which is <V2> in conjunction with any one of <A1> to <U1> above;

<W3> a compound which is <V3> in conjunction with any one of <A1> to <U1> above;

<W4> a compound which is <V4> in conjunction with any one of <A1> to <U1> above; and

<W5> a compound which is <V5> in conjunction with any one of <A1> to <U1>, are preferred.

Furthermore, the respective combinations of substituents for the compound represented by general formula (3) are not particularly limited, but for example, with regard to W, T, R⁶, R⁷, n, V, Ar, R¹ and E, it is preferable that the respective combinations of substituents for the compound represented by the above-described general formula (2) are employed.

With regard to B³¹ and Z³, for example:

<AA1> a compound wherein B³¹ is a divalent group obtained by removing two hydrogen atoms from a 3- to 6-membered saturated ring compound composed of carbon atoms;

<AA2> a compound wherein B³¹ is a divalent group obtained by removing two hydrogen atoms from a 4- to 5-membered saturated ring compound composed of carbon atoms;

<AA3> a compound wherein B³¹ is a divalent group obtained by removing two hydrogen atoms from a 4-membered saturated ring compound composed of carbon atoms;

<AA4> a compound wherein B³¹ is a divalent group obtained by removing two hydrogen atoms from a 5-membered saturated ring compound composed of carbon atoms;

<AA5> the compound according to <AA3>, wherein B³¹ is a divalent group obtained by removing two hydrogen atoms from a 4-membered saturated ring compound composed of carbon atoms, and the positions at which the divalent group is bound to —NR¹— and to -D-E are (1, 3);

<AA6> the compound according to <AA4>, wherein B³¹ is a divalent group obtained by removing two hydrogen atoms from a 5-membered saturated ring compound composed of carbon atoms, and the positions at which the divalent group is bound to —NR¹— and to -D-E are (1, 3);

<AA7> the compound according to <AA3> or <AA5>, wherein the relationship between the bond between B³¹ and —NR¹—, and the bond between B³¹ and -D-E is a cis relationship;

<AA8> the compound according to <AA3> or <AA5>, wherein the relationship between the bond between B³¹ and —NR¹—, and the bond between B³¹ and -D-E is a trans relationship;

<AA9> the compound according to <AA4> or <AA6>, wherein the relationship between the bond between B³¹ and —NR¹—, and the bond between B³¹ and -D-E is a cis relationship;

<AA10> the compound according to <AA4> or <AA6>, wherein the relationship between the bond between B³¹ and —NR¹—, and the bond between B³¹ and -D-E is a trans relationship;

<AB1> a compound wherein Z³ is a C5-C6 cycloalkylene group;

<AB2> a compound wherein Z³ is a divalent group obtained by removing two hydrogen atoms from benzene;

<AB3> a compound wherein Z³ is a divalent group obtained by removing two hydrogen atoms from furan;

<AB4> a compound wherein Z³ is a divalent group obtained by removing two hydrogen atoms from thiophene;

<AB5> a compound wherein Z³ is a divalent group obtained by removing two hydrogen atoms from oxazole;

<AB6> a compound wherein Z³ is a divalent group obtained by removing two hydrogen atoms from isoxazole;

<AB7> a compound wherein Z³ is a divalent group obtained by removing two hydrogen atoms from oxadiazole;

<AB8> a compound wherein Z³ is a divalent group obtained by removing two hydrogen atoms from thiazole;

<AB9> a compound wherein Z³ is a divalent group obtained by removing two hydrogen atoms from isothiazole;

<AB10> a compound wherein Z³ is a divalent group obtained by removing two hydrogen atoms from thiadiazole;

<AB11> a compound wherein Z³ is a divalent group obtained by removing two hydrogen atoms from imidazole;

<AB12> a compound wherein Z³ is a divalent group obtained by removing two hydrogen atoms from pyrazole;

<AB13> a compound wherein Z³ is a divalent group obtained by removing two hydrogen atoms from pyran;

<AB14> a compound wherein Z³ is a divalent group obtained by removing two hydrogen atoms from pyridine;

<AB15> a compound wherein Z³ is a divalent group obtained by removing two hydrogen atoms from pyridazine;

<AB16> a compound wherein Z³ is a divalent group obtained by removing two hydrogen atoms from pyrimidine;

<AB17> a compound wherein Z³ is a divalent group obtained by removing two hydrogen atoms from pyrazine;

<AB18> a compound wherein Z³ is a divalent group obtained by removing two hydrogen atoms from naphthalene;

<AB19> a compound wherein Z³ is a divalent group obtained by removing two hydrogen atoms from benzofuran;

<AB20> a compound wherein Z³ is a divalent group obtained by removing two hydrogen atoms from benzo[b]thiophene;

<AB21> a compound wherein Z³ is a divalent group obtained by removing two hydrogen atoms from indole;

<AB22> a compound wherein Z³ is a divalent group obtained by removing two hydrogen atoms from isoindole;

<AB23> a compound wherein Z³ is a divalent group obtained by removing two hydrogen atoms from indolizine;

<AB24> a compound wherein Z³ is a divalent group obtained by removing two hydrogen atoms from 1H-indazole;

<AB25> a compound wherein Z³ is a divalent group obtained by removing two hydrogen atoms from 2H-indazole;

<AB26> a compound wherein Z³ is a divalent group obtained by removing two hydrogen atoms from I1H-benzimidazole;

<AB27> a compound wherein Z³ is a divalent group obtained by removing two hydrogen atoms from benzoxazole;

<AB28> a compound wherein Z³ is a divalent group obtained by removing two hydrogen atoms from benzo[d]isoxazole;

<AB29> a compound wherein Z³ is a divalent group obtained by removing two hydrogen atoms from benzo[c]isoxazole;

<AB30> a compound wherein Z³ is a divalent group obtained by removing two hydrogen atoms from benzothiazole;

<AB31> a compound wherein Z³ is a divalent group obtained by removing two hydrogen atoms from benzo[d]isothiazole;

<AB32> a compound wherein Z³ is a divalent group obtained by removing two hydrogen atoms from benzo[c]isothiazole;

<AB33> a compound wherein Z³ is a divalent group obtained by removing two hydrogen atoms from 1H-benzotriazole;

<AB34> a compound wherein Z³ is a divalent group obtained by removing two hydrogen atoms from benzo[1,2,5]thiadiazole;

<AB35> a compound wherein Z³ is a divalent group obtained by removing two hydrogen atoms from quinoline;

<AB36> a compound wherein Z³ is a divalent group obtained by removing two hydrogen atoms from isoquinoline;

<AB37> a compound wherein Z³ is substituted with one or two groups each independently selected from the group consisting of a fluorine atom, a chlorine atom and a trifluoromethyl group;

<AC1> a compound which is <AA1> in conjunction with any one of <AB1> to <AB37>;

<AC2> a compound which is <AA2> in conjunction with any one of <AB1> to <AB37>;

<AC3> a compound which is <AA3> in conjunction with any one of <AB1> to <AB37>;

<AC4> a compound which is <AA4> in conjunction with any one of <AB1> to <AB37>;

<AC5> a compound which is <AA5> in conjunction with any one of <AB1> to <AB37>;

<AC6> a compound which is <AA6> in conjunction with any one of <AB1> to <AB37>;

<AC7> a compound which is <AA7> in conjunction with any one of <AB1> to <AB37>;

<AC8> a compound which is <AA8> in conjunction with any one of <AB1> to <AB37>;

<AC9> a compound which is <AA9> in conjunction with any one of <AB1> to <AB37>; and

<AC10> a compound which is <AA10> in conjunction with any one of <AB1> to <AB37>, are preferred.

Furthermore, the respective combinations of substituents for the compound represented by general formula (3) are not particularly limited, but for example, with regard to W, T, R⁶, R⁷, n, V, Ar, R¹, D and E, compounds in which the respective combinations of substituents for the compound represented by the aforementioned general formula (2) are employed, and which are <AA1> to <AC10> are also preferable.

Furthermore, the respective combinations of substituents for the compound represented by general formula (4) are not particularly limited, but for example, with regard to W, T, Z³, R⁶, R⁷, n, V, Ar, R¹, B³¹ and R^{E}, it is preferable that the respective combinations of substituents for the compound represented by the aforementioned general formula (2) are employed.

With regard to B³¹ and Z³, for example:

<BA1> a compound wherein B³¹ is a divalent group obtained by removing two hydrogen atoms from a 3- to 6-membered saturated ring compound composed of carbon atoms;

<BA2> a compound wherein B31 is a divalent group obtained by removing two hydrogen atoms from a 4- to 5-membered saturated ring compound composed of carbon atoms;

<BA3> a compound wherein B31 is a divalent group obtained by removing two hydrogen atoms from a 4-membered saturated ring compound composed of carbon atoms;

<BA4> a compound wherein B31 is a divalent group obtained by removing two hydrogen atoms from a 5-membered saturated ring compound composed of carbon atoms;

<BA5> the compound according to <BA3>, wherein B31 is a divalent group obtained by removing two hydrogen atoms from a 4-membered saturated ring compound composed of carbon atoms, and the positions at which the divalent group is bound to —NR¹— and to —CO₂R^{E} are (1, 3);

<BA6> the compound according to <BA4>, wherein B³¹ is a divalent group obtained by removing two hydrogen atoms from a 5-membered saturated ring compound composed of carbon atoms, and the positions at which the divalent group is bound to —NR¹— and to —CO₂R^{E} are (1, 3);

<BB1> a compound wherein Z³ is a C5-C6 cycloalkylene group;

<BB2> a compound wherein Z³ is a divalent group obtained by removing two hydrogen atoms from benzene;

<BB3> a compound wherein Z³ is a divalent group obtained by removing two hydrogen atoms from furan;

<BB4> a compound wherein Z³ is a divalent group obtained by removing two hydrogen atoms from thiophene;

<BB5> a compound wherein Z³ is a divalent group obtained by removing two hydrogen atoms from oxazole;

<BB6> a compound wherein Z³ is a divalent group obtained by removing two hydrogen atoms from isoxazole;

<BB7> a compound wherein Z³ is a divalent group obtained by removing two hydrogen atoms from oxadiazole;

<BB8> a compound wherein Z³ is a divalent group obtained by removing two hydrogen atoms from thiazole;

<BB9> a compound wherein Z³ is a divalent group obtained by removing two hydrogen atoms from isothiazole;

<BB10> a compound wherein Z³ is a divalent group obtained by removing two hydrogen atoms from thiadiazole;

<BB11> a compound wherein Z³ is a divalent group obtained by removing two hydrogen atoms from imidazole;

<BB12> a compound wherein Z³ is a divalent group obtained by removing two hydrogen atoms from pyrazole;

<BB13> a compound wherein Z³ is a divalent group obtained by removing two hydrogen atoms from pyran;

<BB14> a compound wherein Z³ is a divalent group obtained by removing two hydrogen atoms from pyridine;

<BB15> a compound wherein Z³ is a divalent group obtained by removing two hydrogen atoms from pyridazine;

<BB16> a compound wherein Z³ is a divalent group obtained by removing two hydrogen atoms from pyrimidine;

<BB17> a compound wherein Z³ is a divalent group obtained by removing two hydrogen atoms from pyrazine;

<BB18> a compound wherein Z³ is a divalent group obtained by removing two hydrogen atoms from naphthalene;

<BB19> a compound wherein Z³ is a divalent group obtained by removing two hydrogen atoms from benzofuran;

<BB20> a compound wherein Z³ is a divalent group obtained by removing two hydrogen atoms from benzo[b]thiophene;

<BB21> a compound wherein Z³ is a divalent group obtained by removing two hydrogen atoms from indole;

<BB22> a compound wherein Z³ is a divalent group obtained by removing two hydrogen atoms from isoindole;

<BB23> a compound wherein Z³ is a divalent group obtained by removing two hydrogen atoms from indolizine;

<BB24> a compound wherein Z³ is a divalent group obtained by removing two hydrogen atoms from 1H-indazole;

<BB25> a compound wherein Z³ is a divalent group obtained by removing two hydrogen atoms from 2H-indazole;

<BB26> a compound wherein Z³ is a divalent group obtained by removing two hydrogen atoms from 1H-benzimidazole;

<BB27> a compound wherein Z³ is a divalent group obtained by removing two hydrogen atoms from benzoxazole;

<BB28> a compound wherein Z³ is a divalent group obtained by removing two hydrogen atoms from benzo[c]isoxazole;

<BB29> a compound wherein Z³ is a divalent group obtained by removing two hydrogen atoms from benzo[c]isoxazole;

<BB30> a compound wherein Z³ is a divalent group obtained by removing two hydrogen atoms from benzothiazole;

<BB31> a compound wherein Z³ is a divalent group obtained by removing two hydrogen atoms from benzo[d]isothiazole;

<BB32> a compound wherein Z³ is a divalent group obtained by removing two hydrogen atoms from benzo[c]isothiazole;

<BB33> a compound wherein Z³ is a divalent group obtained by removing two hydrogen atoms from 1H-benzotriazole;

<BB34> a compound wherein Z³ is a divalent group obtained by removing two hydrogen atoms from benzo[1,2,5]thiadiazole;

<BB35> a compound wherein Z³ is a divalent group obtained by removing two hydrogen atoms from quinoline;

<BB36> a compound wherein Z³ is a divalent group obtained by removing two hydrogen atoms from isoquinoline;

<BB37> a compound wherein Z³ is substituted with one or two groups each independently selected from the group consisting of a fluorine atom, a chlorine atom and a trifluoromethyl group;

<BC1> a compound which is <BA1> in conjunction with any one of <BB1> to <BB37>;

<BC2> a compound which is <BA2> in conjunction with any one of <BB1> to <BB37>;

<BC3> a compound which is <BA3> in conjunction with any one of <BB1> to <BB37>;

<BC4> a compound which is <BA4> in conjunction with any one of <BB1> to <BB37>;

<BC5> a compound which is <BA5> in conjunction with any one of <BB1> to <BB37>; and

<BC6> a compound which is <BA6> in conjunction with any one of <BB1> to <BB37>, are preferred.

Also, the respective combinations of substituents for the compound represented by general formula (4) are not particularly limited, but for example, with regard to W, T, Z³, R⁶, R⁷, n, V, Ar, R¹, B³¹ and R^{E}, compounds in which the respective combinations of substituents for the compound represented by the aforementioned general formula (2) are employed, and which are <BA1> to <BC6> are also preferable. Furthermore, the respective combinations of substituents for the compound represented by general formula (5) are not particularly limited, but for example, with regard to W, T, Z³, R⁶, R⁷, n, V, R¹, B³¹ and R^{E}, it is preferable that the respective combinations of substituents for the compound represented by the aforementioned general formula (2) are employed.

With regard to B³¹ and Z³, for example:

<CA1> a compound wherein B³¹ is a divalent group obtained by removing two hydrogen atoms from a 3- to 6-membered saturated ring compound composed of carbon atoms;

<CA2> a compound wherein B³¹ is a divalent group obtained by removing two hydrogen atoms from a 4- to 5-membered saturated ring compound composed of carbon atoms;

<CA3> a compound wherein B³¹ is a divalent group obtained by removing two hydrogen atoms from a 4-membered saturated ring compound composed of carbon atoms;

<CA4> a compound wherein B³¹ is a divalent group obtained by removing two hydrogen atoms from a 5-membered saturated ring compound composed of carbon atoms;

<CA5> the compound according to <CA3>, wherein B³¹ is a divalent group obtained by removing two hydrogen atoms from a 4-membered saturated ring compound composed of carbon atoms, and the positions at which the divalent group is bound to —NR¹— and to —CO₂R^{E} are (1, 3);

<CA6> the compound according to <CA4>, wherein B³¹ is a divalent group obtained by removing two hydrogen atoms from a 5-membered saturated ring compound composed of carbon atoms, and the positions at which the divalent group is bound to —NR¹— and to —CO₂R^{E} are (1, 3);

<CA7> the compound according to <CA3> or <CA5>, wherein the relationship between the bond between B³¹ and —NR¹—, and the bond between B³¹ and -D-E is a cis relationship;

<CA8> the compound according to <CA3> or <CA5>, wherein the relationship between the bond between B³¹ and —NR¹—, and the bond between B31 and -D-E is a trans relationship;

<CA9> the compound according to <CA4> or <CA6>, wherein the relationship between the bond between B³¹ and —NR¹—, and the bond between B³¹ and -D-E is a cis relationship;

<CA10> the compound according to <CA4> or <CA6>, wherein the relationship between the bond between B³¹ and —NR¹—, and the bond between B³¹ and -D-E is a trans relationship;

<CB1> a compound wherein Z³ is a C5-C6 cycloalkylene group;

<CB2> a compound wherein Z³ is a divalent group obtained by removing two hydrogen atoms from benzene;

<CB3> a compound wherein Z³ is a divalent group obtained by removing two hydrogen atoms from furan;

<CB4> a compound wherein Z³ is a divalent group obtained by removing two hydrogen atoms from thiophene;

<CB5> a compound wherein Z³ is a divalent group obtained by removing two hydrogen atoms from oxazole;

<CB6> a compound wherein Z³ is a divalent group obtained by removing two hydrogen atoms from isoxazole;

<CB7> a compound wherein Z³ is a divalent group obtained by removing two hydrogen atoms from oxadiazole;

<CB8> a compound wherein Z³ is a divalent group obtained by removing two hydrogen atoms from thiazole;

<CB9> a compound wherein Z³ is a divalent group obtained by removing two hydrogen atoms from isothiazole;

<CB10> a compound wherein Z³ is a divalent group obtained by removing two hydrogen atoms from thiadiazole;

<CB11> a compound wherein Z³ is a divalent group obtained by removing two hydrogen atoms from imidazole;

<CB12> a compound wherein Z³ is a divalent group obtained by removing two hydrogen atoms from pyrazole;

<CB13> a compound wherein Z³ is a divalent group obtained by removing two hydrogen atoms from pyran;

<CB14> a compound wherein Z³ is a divalent group obtained by removing two hydrogen atoms from pyridine;

<CB15> a compound wherein Z³ is a divalent group obtained by removing two hydrogen atoms from pyridazine;

<CB16> a compound wherein Z³ is a divalent group obtained by removing two hydrogen atoms from pyrimidine;

<CB17> a compound wherein Z³ is a divalent group obtained by removing two hydrogen atoms from pyrazine;

<CB18> a compound wherein Z³ is a divalent group obtained by removing two hydrogen atoms from naphthalene;

<CB19> a compound wherein Z³ is a divalent group obtained by removing two hydrogen atoms from benzofuran;

<CB20> a compound wherein Z³ is a divalent group obtained by removing two hydrogen atoms from benzo[b]thiophene;

<CB21> a compound wherein Z³ is a divalent group obtained by removing two hydrogen atoms from indole;

<CB22> a compound wherein Z³ is a divalent group obtained by removing two hydrogen atoms from isoindole;

<CB23> a compound wherein Z³ is a divalent group obtained by removing two hydrogen atoms from indolizine;

<CB24> a compound wherein Z³ is a divalent group obtained by removing two hydrogen atoms from 1H-indazole;

<CB25> a compound wherein Z³ is a divalent group obtained by removing two hydrogen atoms from 2H-indazole;

<CB26> a compound wherein Z³ is a divalent group obtained by removing two hydrogen atoms from 1H-benzimidazole;

<CB27> a compound wherein Z³ is a divalent group obtained by removing two hydrogen atoms from benzoxazole;

<CB28> a compound wherein Z³ is a divalent group obtained by removing two hydrogen atoms from benzo[d]isoxazole;

<CB29> a compound wherein Z³ is a divalent group obtained by removing two hydrogen atoms from benzo[c]isoxazole;

<CB30> a compound wherein Z³ is a divalent group obtained by removing two hydrogen atoms from benzothiazole;

<CB31> a compound wherein Z³ is a divalent group obtained by removing two hydrogen atoms from benzo[d]isothiazole;

<CB32> a compound wherein Z³ is a divalent group obtained by removing two hydrogen atoms from benzo[c]isothiazole;

<CB33> a compound wherein Z³ is a divalent group obtained by removing two hydrogen atoms from 1H-benzotriazole;

<CB34> a compound wherein Z³ is a divalent group obtained by removing two hydrogen atoms from benzo[1,2,5]thiadiazole;

<CB35> a compound wherein Z³ is a divalent group obtained by removing two hydrogen atoms from quinoline;

<CB36> a compound wherein Z³ is a divalent group obtained by removing two hydrogen atoms from isoquinoline;

<CB37> a compound wherein Z³ is substituted with one or two groups each independently selected from the group consisting of a fluorine atom, a chlorine atom and a trifluoromethyl group;

<CC1> a compound which is <CA1> in conjunction with any one of <CB1> to <CB37>;

<CC2> a compound which is <CA2> in conjunction with any one of <CB1> to <CB37>;

<CC3> a compound which is <CA3> in conjunction with any one of <CB1> to <CB37>;

<CC4> a compound which is <CA4> in conjunction with any one of <CB1> to <CB37>;

<CC5> a compound which is <CA5> in conjunction with any one of <CB1> to <CB37>;

<CC6> a compound which is <CA6> in conjunction with any one of <CB1> to <CB37>;

<CC7> a compound which is <CA7> in conjunction with any one of <CB1> to <CB37>;

<CC8> a compound which is <CA8> in conjunction with any one of <CB1> to <CB37>;

<CC9> a compound which is <CA9> in conjunction with any one of <CB1> to <CB37>; and

<CC10> a compound which is <CA10> in conjunction with any one of <CB1> to <CB37>, are preferred.

Furthermore, the respective combinations of substituents for the compound represented by general formula (5) are not particularly limited, but for example, with regard to W, T, Z³, R⁶, R⁷, n, V, R¹, B³¹ and R^{E}, compounds in which the respective combinations of substituents for the compound represented by the aforementioned general formula (2) are employed, and which are <CA1> to <CC6> are also preferable.

Also, the compound represented by the general formula (2); or the compounds of <A1> to <W5> in which the respective combinations of substituents for the compound represented by the general formula (2) have been defined; or the compound represented by the general formula (3), the general formula (3B), the general formula (4), the general formula (5), or the general formula (6); or the compounds described above, in which the respective combinations of substituents for the compound represented by the general formula (3), the general formula (3B), the general formula (4), the general formula (5), or the general formula (6) have been defined; and salts thereof;

pharmacologically acceptable salts of the compound represented by the general formula (2); or of the compounds of <A1> to <W5>, in which the respective combinations of substituents for the compound represented by the general formula (2) have been defined; or of the compound represented by the general formula (3), the general formula (3B), the general formula (4), the general formula (5), or the general formula (6); or of the compounds described above, in which the respective combinations of substituents for the compound represented by the general formula (3), the general formula (3B), the general formula (4), the general formula (5), or the general formula (6) have been defined;

prodrugs of the compound represented by general formula (2); or the compounds of <A1> to <W5>, which compounds define the respective combinations of substituents for the compound represented by general formula (2); or of the compounds represented by general formula (3), general formula (3B), general formula (4), general formula (5) or the compound represented by general formula (6); or of the compounds described above, which define the respective combinations of substituents for the compounds represented by general formula (3), general formula (3B), general formula (4), general formula (5) or general formula (6);

prodrugs of the salts of the compound represented by general formula (2); or of the compounds of <A1> to <W5>, which compounds define the respective combinations of substituents for the compound represented by general formula (2); or of the compounds represented by general formula (3), general formula (3B), general formula (4), general formula (5) or general formula (6); or the compounds described above which define the respective combinations of substituents for the compounds represented by general formula (3), general formula (3B), general formula (4), general formula (5) or general formula (6); or

prodrugs of the pharmacologically acceptable salts of the compound represented by general formula (2); or the compounds <A1> to <W5>, which compounds define the respective combinations of substituents for the compound represented by general formula (2); or of the compounds represented by general formula (3), general formula (3B), general formula (4), general formula (5) or general formula (6); or the compounds described above which define the respective combinations of substituents for the compounds represented by general formula (3), general formula (3B), general formula (4), general formula (5) or general formula (6), are preferable.

Furthermore, the following pharmaceutical products, pharmaceutical compositions and methods for treatment using the compounds represented by general formulas (1) to (6), or the compounds described above which define the respective combinations of substituents for the compounds described above which define the respective combinations of substituents for the compounds represented by general formulas (2) to (6), or the possible stereoisomers or racemic bodies thereof, or pharmacologically acceptable salts, hydrates, solvates, or prodrugs of the compounds, the stereoisomers or the racemic bodies, or prodrugs thereof are also within the scope of the present invention. The compounds represented by the general formulas (1) to (6), or the compounds represented by the general formulas (2) to (6) also include the compounds represented by the general formula (3B).

<DA1> A pharmaceutical product for the prevention and/or treatment of allergic diseases of mammals, comprising, as an active ingredient, the compounds represented by general formulas (1) to (6) or the compounds described above which define the respective combinations of substituents for the compounds represented by general formulas (2) to (6), or possible stereoisomers or racemic bodies thereof, or pharmacologically acceptable salts, hydrates, solvates of the compounds, the stereoisomers or the racemic bodies, or prodrugs thereof.

<DA2> A pharmaceutical composition for the prevention and/or treatment of a biological condition in a mammal, in which condition acute or chronic autoimmune diseases are recognized, which composition comprises the compounds represented by general formulas (1) to (6) or the compounds described above which define the respective combinations of substituents for the compounds represented by general formulas (2) to (6), or possible stereoisomers or racematic bodies thereof, or pharmacologically acceptable salts, hydrates, solvates of the compounds, the sterecisomers or the racemic doeis, or prodrugs thereof, in an amount effective for the prevention and/or treatment.

<DA3> A method for preventing and/or treating a biological condition in a mammal, in which acute or chronic autoimmune diseases are recognized, which method comprises administering to the mammal the compounds represented by general formulas (1) to (6) or the compounds described above which define the respective combinations of substituents for the compounds represented by general formulas (2) to (6), or possible stereoisomers or racemic bodies thereof, or pharmacologically acceptable salts, hydrates, solvates of the compounds, the stereoisomers or the racemic bodies, or prodrugs thereof, in an amount effective for the prevention and/or treatment.

In addition, the respective combinations of substituents for the compound represented by general formula (3) are not particularly limited, but for example:

<EA1> a compound wherein R¹ is a hydrogen atom;

<EA2> a compound wherein R¹ is a methyl group;

<EA3> a compound wherein R¹ is an ethyl group;

<EB1> a compound wherein X¹ is a methyl group;

<EB2> a compound wherein X¹ is an ethyl group;

<EB3> a compound wherein X¹ is a methoxy group;

<EB4> a compound wherein X¹ is a fluorine atom;

<EB5> a compound wherein X¹ is a chlorine atom;

<EB6> a compound wherein X¹ is a bromine atom;

<EC1> a compound which is <EA1> in conjunction with any one of <EB1> to <EB6>;

<EC2> a compound which is <EA2> in conjunction with any one of <EB1> to <EB6>;

<EC3> a compound which is <EA3> in conjunction with any one of <EB1> to <EB6>;

<ED1> a compound wherein Ar is a divalent group obtained by removing two hydrogen atoms from benzene;

<ED2> a compound wherein Ar is a divalent group obtained by removing two hydrogen atoms from thiophene;

<ED3> a compound wherein Ar is a divalent group obtained by removing two hydrogen atoms from pyridine;

<ED4> the compound according to <ED1>, wherein Ar is a divalent group obtained by removing two hydrogen atoms from benzene, and the positions at which the divalent group is bound to W-T-Z³-(CR⁶R⁷)ₙ—V¹- and to CH₂—N(R¹)B—CO₂R^{E} are (1, 4);

<ED5> the compound according to <ED2>, wherein Ar is a divalent group obtained by removing two hydrogen atoms from thiophene, and the positions at which the divalent group is bound to W-T-Z³-(CR⁶R⁷)ₙ—V¹- and to —CH₂—N(R¹)B—CO₂R^{E} are (2, 5);

<ED6> the compound according to <ED3>, wherein Ar is a divalent group obtained by removing two hydrogen atoms from pyridine, and the positions at which the divalent group is bound to W-T-Z³-(CR⁶R⁷)ₙ—V¹- and to —CH₂—N(R¹)B—CO₂R^{E} are (2, 5), or (3, 6);

<EE1> a compound which is <ED1> in conjunction with any one of <EA1> to <EC3>;

<EE2> a compound which is <ED2> in conjunction with any one of <EA1> to <EC3>;

<EE3> a compound which is <ED3> in conjunction with any one of <EA1> to <EC3>;

<EE4> a compound which is <ED4> in conjunction with any one of <EA1> to <EC3>;

<EE5> a compound which is <ED5> in conjunction with any one of <EA1> to <EC3>;

<EE6> a compound which is <ED6> in conjunction with any one of <EA1> to <EC3>;

<EF1> a compound wherein B³¹ is a divalent group obtained by removing two hydrogen atoms from a 4- to 5-membered saturated ring compound composed of carbon atoms;

<EF2> a compound wherein B³¹ is a divalent group obtained by removing two hydrogen atoms from a 4-membered saturated ring compound composed of carbon atoms;

<EF3> a compound wherein B³¹ is a divalent group obtained by removing two hydrogen atoms from a 5-membered saturated ring compound composed of carbon atoms;

<EF4> the compound according to <EF2>, wherein B³¹ is a divalent group obtained by removing two hydrogen atoms from a 4-membered saturated ring compound composed of carbon atoms, and the positions at which the divalent group is bound to —NR¹- and to -D-E are (1,3);

<EF5> the compound according to <EF3>, wherein B³¹ is a divalent group obtained by removing two hydrogen atoms from a 5-membered saturated ring compound composed of carbon atoms, and the positions at which the divalent group is bound to —NR¹— and to -D-E are (1,3);

<EF6> the compound according to <EF2> or <EF4>, wherein the relationship between the bond between B³¹ and —NR¹-, and the bond between B³¹ and -D-E is a cis relationship;

<EF7> the compound according to <EF3> or <EF5>, wherein the relationship between the bond between B³¹ and —NR¹—, and the bond between B³¹ and -D-E is a trans relationship;

<EG1> a compound which is <EF1> in conjunction with any one of <EA1> to <EE6>;

<EG2> a compound which is <EF2> in conjunction with any one of <EA1> to <EE6>;

<EG3> a compound which is <EF3> in conjunction with any one of <EA1> to <EE6>;

<EG4> a compound which is <EF4> in conjunction with any one of <EA1> to <EE6>;

<EG5> a compound which is <EF5> in conjunction with any one of <EA1> to <EE6>;

<EG6> a compound which is <EF6> in conjunction with any one of <EA1> to <EE6>;

<EG7> a compound which is <EF7> in conjunction with any one of <EA1> to <EE6>;

<EH1> a compound wherein D is a single bond;

<EH2> a compound wherein D is a methylene group;

<E1> a compound which is <EH1> in conjunction with any one <EA1> to <EG7>;

<EI2> a compound which is <EH2> in conjunction with any one <EA1> to <EG7>;

<EJ1> a compound wherein E is —CO₂R^{E};

<EJ2> a compound wherein E is —CO₂H;

<EJ3> a compound wherein E is —CO₂Me;

<EJ4> a compound wherein E is —CO₂Et;

<EK1> a compound which is <EJ1> in conjunction with any one of <EA1> to <EI2>;

<EK2> a compound which is <EJ2> in conjunction with any one of <EA1> to <EI2>;

<EK3> a compound which is <EJ3> in conjunction with any one of <EA1> to <EI2>;

<EK4> a compound which is <EJ4> in conjunction with any one of <EA1> to <EI2>;

<EL1> a compound wherein X⁴ is a fluorine atom;

<EL2> a compound wherein X⁴ is a cyano group;

<EL3> a compound wherein X⁴ is a methyl group;

<EL4> a compound wherein X⁴ is a trifluoromethoxy group;

<EM1> a compound which is <EL1> in conjunction with any one of <EA1> to <EK4>;

<EM2> a compound which is <EL2> in conjunction with any one of <EA1> to <EK4>;

<EM3> a compound which is <EL3> in conjunction with any one of <EA1> to <EK4>;

<EM4> a compound which is <EL4> in conjunction with any one of <EA1> to <EK4>;

<EN1> a compound wherein W is a C1-C6 alkyl group which may be substituted with 1 to 7 fluorine atoms;

<EN2> a compound wherein W is a C5-C6 alkyl group which may be substituted with 1 to 7 fluorine atoms;

<EN3> a compound wherein W is a C3-C7 cycloalkyl group which may be substituted with 1 to 7 fluorine atoms;

<EN4> a compound wherein W is a C5-C7 cycloalkyl group which may be substituted with 1 to 7 fluorine atoms;

<EN5> a compound wherein W is a monovalent group obtained by removing one hydrogen atom from benzene;

<EN6> a compound wherein W is a monovalent group obtained by removing one hydrogen atom from naphthalene;

<EN7> a compound wherein W is a monovalent group obtained by removing one hydrogen atom from furan;

<EN8> a compound wherein W is a monovalent group obtained by removing one hydrogen atom from thiophene;

<EN9> a compound wherein W is a monovalent group obtained by removing one hydrogen atom from pyrrole;

<EN10> a compound wherein W is a monovalent group obtained by removing one hydrogen atom from oxazole;

<EN11> a compound wherein W is a monovalent group obtained by removing one hydrogen atom from isoxazole;

<EN12> a compound wherein W is a monovalent group obtained by removing one hydrogen atom from oxadiazole;

<EN13> a compound wherein W is a monovalent group obtained by removing one hydrogen atom from thiazole;

<EN14> a compound wherein W is a monovalent group obtained by removing one hydrogen atom from isothiazole;

<EN15> a compound wherein W is a monovalent group obtained by removing one hydrogen atom from thiadiazole;

<EN16> a compound wherein w is a monovalent group obtained by removing one hydrogen atom from imidazole;

<EN17> a compound wherein W is a monovalent group obtained by removing one hydrogen atom from pyrazole;

<EN18> a compound wherein W is a monovalent group obtained by removing one hydrogen atom from imidazole;

<EN19> a compound wherein W is a monovalent group obtained by removing one hydrogen atom from pyrazole;

<EN20> a compound wherein W is a monovalent group obtained by removing one hydrogen atom from pyran;

<EN21> a compound wherein W is a monovalent group obtained by removing one hydrogen atom from pyridine;

<EN22> a compound wherein W is a monovalent group obtained by removing one hydrogen atom from pyridazine;

<EN23> a compound wherein W is a monovalent group obtained by removing one hydrogen atom from pyrimidine;

<EN24> a compound wherein W is a monovalent group obtained by removing one hydrogen atom from pyrazine;

<EN25> a compound wherein W is a monovalent group obtained by removing one hydrogen atom from oxazine;

<EN26> a compound wherein W is a monovalent group obtained by removing one hydrogen atom from thiazine;

<EO1> a compound which is <EN1> in conjunction with any one of <EA1> to <EM4>;

<EO2> a compound which is <EN2> in conjunction with any one of <EA1> to <EM4>;

<EO3> a compound which is <EN3> in conjunction with any one of <EA1> to <EM4>;

<EO4> a compound which is <EN4> in conjunction with any one of <EA1> to <EM4> above;

<EO5> a compound which is <EN5> in conjunction with any one of <EA1> to <EM4>;

<EO6> a compound which is <EN6> in conjunction with any one of <EA1> to <EM4>;

<EO7> a compound which is <EN7> in conjunction with any one of <EA1> to <EM4>;

<EO8> a compound which is <EN8> in conjunction with any one of <EA1> to <EM4>;

<EO9> a compound which is <EN9> in conjunction with any one of <EA1> to <EM4>;

<EO10> a compound which is <EN10> in conjunction with any one of <EA1> to <EM4>;

<EO11> a compound which is <EN11> in conjunction with any one of <EA1> to <EM4>;

<EO12> a compound which is <EN12> in conjunction with any one of <EA1> to <EM4>;

<EO13> a compound which is <EN13> in conjunction with any one of <EA1> to <EM4>;

<EO14> a compound which is <EN14> in conjunction with any one of <EA1> to <EM4>;

<EO15> a compound which is <EN15> in conjunction with any one of <EA1> to <EM4>;

<EO16> a compound which is <EN16> in conjunction with any one of <EA1> to <EM4>;

<EO17> a compound which is <EN17> in conjunction with any one of <EA1> to <EM4>;

<EO18> a compound which is <EN18> in conjunction with any one of <EA1> to <EM4>;

<EO19> a compound which is <EN19> in conjunction with any one of <EA1> to <EM4>;

<EO20> a compound which is <EN20> in conjunction with any one of <EA1> to <EM4>;

<EO21> a compound which is <EN21> in conjunction with any one of <EA1> to <EM4>;

<EO22> a compound which is <EN22> in conjunction with any one of <EA1> to <EM4>;

<EO23> a compound which is <EN23> in conjunction with any one of <EA1> to <EM4>;

<EO24> a compound which is <EN24> in conjunction with any one of <EA1> to <EM4>;

<EO25> a compound which is <EN2s> in conjunction with any one of <EA1> to <EM4>;

<EO26> a compound which is <EN26> in conjunction with any one of <EA1> to <EM4>;

<EP1> a compound wherein T is a single bond;

<EP2> a compound wherein T is a methylene group;

<EP3> a compound wherein T is an ethylene group;

<EP4> a compound wherein T is —O—;

<EP5> a compound wherein T is —CONR^{T}—;

<EQ1> a compound which is <EP1> in conjunction with any one of <EA1> to <EO26>;

<EQ2> a compound which is <EP2> in conjunction with any one of <EA1> to <EO²⁶>;

<EQ3> a compound which is <EP3> in conjunction with any one of <EA1> to <EO26>;

<EQ4> a compound which is <EP4> in conjunction with any one of <EA1> to <EO26>;

<EQ5> a compound which is <EP5> in conjunction with any one of <EA1> to <EO26>;

<ER1> a compound wherein X^{Z3} is a fluorine atom;

<ER2> a compound wherein X^{Z3} is a chlorine atom;

<ER3> a compound wherein X^{Z3} is a trifluoromethyl group;

<ER4> a compound wherein X^{Z3} is a C1-C6 alkyl group substituted with a fluorine atom;

<ER5> a compound wherein X^{Z3} is a cyano group;

<ER6> a compound wherein X^{Z3} is a methoxy group;

<ER7> a compound wherein X^{Z3} is a methyl group;

<ES1> a compound which is <ER1 > in conjunction with any one of <EA1> to <EQ5>;

<ES2> a compound which is <ER2> in conjunction with any one of <EA1> to <EQ5>;

<ES3> a compound which is <ER3> in conjunction with any one of <EA1> to <EQ5>;

<ES4> a compound which is <ER4> in conjunction with any one of <EA1> to <EQ5>;

<ES5> a compound which is <ER5> in conjunction with any one of <EA1> to <EQ5>;

<ES6> a compound which is <ER6> in conjunction with any one of <EA1> to <EQ5>;

<ES7> a compound which is <ER7> in conjunction with any one of <EA1> to <EQ5>;

<ET1> a compound wherein Z³ is a C5-C6 cycloalkylene group;

<ET2> a compound wherein Z³ is a divalent group obtained by removing two hydrogen atoms from benzene;

<ET3> a compound wherein Z³ is a divalent group obtained by removing two hydrogen atoms from furan;

<ET4> a compound wherein Z³ is a divalent group obtained by removing two hydrogen atoms from thiophene;

<ET5> a compound wherein Z³ is a divalent group obtained by removing two hydrogen atoms from oxazole;

<ET6> a compound wherein Z³ is a divalent group obtained by removing two hydrogen atoms from isoxazole;

<ET7> a compound wherein Z³ is a divalent group obtained by removing two hydrogen atoms from oxadiazole;

<ET8> a compound wherein Z³ is a divalent group obtained by removing two hydrogen atoms from thiazole;

<ET9> a compound wherein Z³ is a divalent group obtained by removing two hydrogen atoms from isothiazole;

<ET10> a compound wherein Z³ is a divalent group obtained by removing two hydrogen atoms from thiadiazole;

<ET11> a compound wherein Z³ is a divalent group obtained by removing two hydrogen atoms from imidazole;

<ET12> a compound wherein Z³ is a divalent group obtained by removing two hydrogen atoms from pyrazole;

<ET13> a compound wherein Z³ is a divalent group obtained by removing two hydrogen atoms from pyran;

<ET14> a compound wherein Z³ is a divalent group obtained by removing two hydrogen atoms from pyridine;

<ET15> a compound wherein Z³ is a divalent group obtained by removing two hydrogen atoms from pyridazine;

<ET16> a compound wherein Z³ is a divalent group obtained by removing two hydrogen atoms from pyrimidine;

<ET17> a compound wherein Z³ is a divalent group obtained by removing two hydrogen atoms from pyrazine;

<EU1> a compound which is <ET1> in conjunction with any one of <EA1> to <ES7>;

<EU2> a compound which is <ET2> in conjunction with any one of <EA1> to <ES7>;

<EU3> a compound which is <ET3> in conjunction with any one of <EA1> to <ES7>;

<EU4> a compound which is <ET4> in conjunction with any one of <EA1> to <ES7>;

<EU5> a compound which is <ET5> in conjunction with any one of <EA1> to <ES7>;

<EU6> a compound which is <ET6> in conjunction with any one of <EA1> to <ES7>;

<EU7> a compound which is <ET7> in conjunction with any one of <EA1> to <ES7>;

<EU8> a compound which is <ET8> in conjunction with any one of <EA1> to <ES7>;

<EU9> a compound which is <ET9> in conjunction with any one of <EA1> to <ES7>;

<EU10> a compound which is <ET10> in conjunction with any one of <EA1> to <ES7>;

<EU11> a compound which is <ET11> in conjunction with any one of <EA1> to <ES7>;

<EU12> a compound which is <ET12> in conjunction with any one of <EA1> to <ES7>;

<EU13> a compound which is <ET13> in conjunction with any one of <EA1> to <ES7>;

<EU14> a compound which is <ET14> in conjunction with any one of <EA1> to <ES7>;

<EU15> a compound which is <ET15> in conjunction with any one of <EA1> to <ES7>;

<EU16> a compound which is <ET16> in conjunction with any one of <EA1> to <ES7>;

<EU17> a compound which is <ET17> in conjunction with any one of <EA1> to <ES7>;

<EV1> a compound wherein V¹ is a single bond;

<EV2> a compound wherein V¹ is —O—;

<EV3> a compound wherein V¹ is —CO—;

<EV4> a compound wherein V¹ is —CR^{V1} (OCR^{V12})CR^{V13}CR^{V14}—;

<EV5> a compound wherein V¹ is —CR^{V1}═CR^{V12}—;

<EV6> a compound wherein V¹ is —C≡C—;

<EV7> a compound wherein V¹ is —CONR^{V1}—;

<EV8> a compound wherein V¹ is a divalent group obtained by removing two hydrogen atoms from [1,2,4]-oxadiazole;

<EV9> a compound wherein V¹ is a divalent group obtained by removing two hydrogen atoms from [1,3,4]-oxadiazole;

<EV10> a compound wherein V¹ is a divalent group obtained by removing two hydrogen atoms from [1,2,4]-thiadiazole;

<EV11> a compound wherein V¹ is a divalent group obtained by removing two hydrogen atoms from [1,3,4]-thiadiazole;

<EV12> the compound according to <EV8>, wherein the positions at which the divalent group obtained by removing two hydrogen atoms from [1,2,4]-oxadiazole is bound to W-T-Z³-(CR⁶R⁷)ₙ— and to —Ar—CH₂—NR¹—B^{B31}-D-E are (5, 3);

<EV13> the compound according to <EV8>, wherein the positions at which the divalent group obtained by removing two hydrogen atoms from [1,2,4]-oxadiazole is bound to W-T-Z³-(CR⁶R⁷)ₙ— and to —Ar—CH₂—NR¹—B^{B31}-D-E are (3, 5);

<EV14> the compound according to <EV10>, wherein the positions at which the divalent group obtained by removing two hydrogen atoms from [1,2,4]-thiadiazole is bound to W-T-Z³-(CR⁶R⁷)ₙ— and to —Ar—CH₂—NR¹—B^{B31}-D-E are (5, 3);

<EV15> the compound according to <EV10>, wherein the positions at which the divalent group obtained by removing two hydrogen atoms from [1,2,4]-thiadiazole is bound to W-T-Z³-(CR⁶R⁷)ₙ— and to —Ar—CH₂—NR¹—B^{B31}-D-E are (3, 5);

<EW1> a compound which is <EV1> in conjunction with any one of <EA1> to <EU17>;

<EW2> a compound which is <EV2> in conjunction with any one of <EA1> to <EU17>;

<EW3> a compound which is <EV3> in conjunction with any one of <EA1> to <EU17>;

<EW4> a compound which is <EV4> in conjunction with any one of <EA1> to <EU17>;

<EW5> a compound which is <EV5> in conjunction with any one of <EA1> to <EU17>;

<EW6> a compound which is <EV6> in conjunction with any one of <EA1> to <EU17>;

<EW7> a compound which is <EV7> in conjunction with any one of <EA1> to <EU17>;

<EW8> a compound which is <EV8> in conjunction with any one of <EA1> to <EU17>;

<EW9> a compound which is <EV9> in conjunction with any one of <EA1> to <EU17>;

<EW10> a compound which is <EV10> in conjunction with any one of <EA1> to <EU17>;

<EW11> a compound which is <EV11> in conjunction with any one of <EA1> to <EU17>;

<EW12> a compound which is <EV12> in conjunction with any one of <EA1> to <EU17>;

<EW13> a compound which is <EV13> in conjunction with any one of <EA1> to <EU17>;

<EW14> a compound which is <EV14> in conjunction with any one of <EA1> to <EU17>;

<EW15> a compound which is <EV15> in conjunction with any one of <EA1> to <EU17>;

<EX1> a compound wherein n is 0;

<EX2> a compound wherein n is 1;

<EY1> a compound which is <EX1> in conjunction with any one of <EA1> to <EW15>; and

<EY2> a compound which is <EX2> in conjunction with any one of <EA1> to <EW15>, are preferable.

The respective combinations of substituents for the compound represented by general formula (6) are not particularly limited, but for example:

<ZA1> a compound wherein R^{1B} is a hydrogen atom;

<ZA2> a compound wherein R^{1B} is a methyl group;

<ZA3> a compound wherein R^{1B} is an ethyl group;

<ZB1> a compound wherein X^{1B} is a methyl group;

<ZB2> a compound wherein X^{1B} is an ethyl group;

<ZB3> a compound wherein X^{1B} is a methoxy group;

<ZB4> a compound wherein X^{1B} is a fluorine atom;

<ZB5> a compound wherein X^{1B} is a chlorine atom;

<ZB6> a compound wherein X^{1B} is a bromine atom;

<ZB7> a compound which is anyone of <ZB1> to <ZB6>, wherein when there are two X^{1B}s, one of X^{1B}s is a methyl group;

<ZB8> a compound which is any one of <ZB1> to <ZB6>, wherein when there are two X^{1B}s, one of X^{1B}s is an ethyl group;

<ZC1> a compound which is <ZB1> in conjunction with any one of <ZA1> to <ZA3>;

<ZC2> a compound which is <ZB2> in conjunction with any one of <ZA1> to <ZA3>;

<ZC3> a compound which is <ZB3> in conjunction with any one of <ZA1> to <ZA3>;

<ZC4> a compound which is <ZB4> in conjunction with any one of <ZA1> to <ZA3>;

<ZC5> a compound which is <ZB5> in conjunction with any one of <ZA1> to <ZA3>;

<ZC6> a compound which is <ZB6> in conjunction with any one of <ZA1> to <ZA3>;

<ZC7> a compound which is <ZB7> in conjunction with any one of <ZA1> to <ZA3>;

<ZC8> a compound which is <ZB8> in conjunction with any one of <ZA1> to <ZA3>;

<ZD1> a compound wherein Ar⁸ is a divalent group obtained by removing two hydrogen atoms from benzene;

<ZD2> a compound wherein Ar⁸ is a divalent group obtained by removing two hydrogen atoms from thiophene;

<ZD3> a compound wherein Ar⁸ is a divalent group obtained by removing two hydrogen atoms from pyridine;

<ZD4> the compound according to <ZD1>, wherein Ar⁸ is a divalent group obtained by removing two hydrogen atoms from benzene, and the positions at which the divalent group is bound to W^{B}-T^{B}-Z^{3B}-(CR^{6B}R^{7B})ₙ^{B}—V^{1B}-, and to —CH₂—N(R^{1B}) B^{B}—CO₂R^{EB} are (1, 4);

<ZD5> the compound according to <ZD2>, wherein Ar^{B} is a divalent group obtained by removing two hydrogen atoms from thiophene, and the positions at which the divalent group is bound to W^{B}-T^{B}-Z^{3B}-(CR^{6B}R^{7B})ₙ^{B}—V^{1B}, and to —CH₂—N(R^{1B})B^{B}—CO₂R^{EB} are (2, 5);

<ZD6> the compound according to <ZD3>, wherein Ar^{B} is a divalent group obtained by removing two hydrogen atoms from pyridine, and the positions at which the divalent group is bound to W^{B}-T^{B}-Z^{3B}-(CR^{6B}R^{7B})ₙ^{B}—V^{1B}, and to —CH₂—N(R^{1B})B^{B}—CO₂R^{EB} are (2, 5) or (3, 6);

<ZE1> a compound which is <ZD1> in conjunction with any one of <ZA1> to <ZC8>;

<ZE2> a compound which is <ZD2> in conjunction with any one of <ZA1> to <ZC8>;

<ZE3> a compound which is <ZD3> in conjunction with any one of <ZA1> to <ZC8>;

<ZE4> a compound which is <ZD4> in conjunction with any one of <ZA1> to <ZC8>;

<ZE5> a compound which is <ZD5> in conjunction with any one of <ZA1> to <ZC8>;

<ZE6> a compound which is <ZD6> in conjunction with any one of <ZA1> to <ZC8>;

<ZF1> a compound wherein B^{B} is an unsubstituted C2 alkylene group;

<ZF2> a compound wherein B^{B} is a C2 alkylene group substituted with a fluorine atom;

<ZF3> a compound wherein BB is a C2 alkylene group substituted with a methyl group;

<ZF4> a compound wherein B^{B} is a C2 alkylene group substituted with an ethyl group;

<ZG1> a compound which is <ZF1> in conjunction with any one of <ZA1> to <ZE6>;

<ZG2> a compound which is <ZF2> in conjunction with any one of <ZA1> to <ZE6>;

<ZG3> a compound which is <ZF3> in conjunction with any one of <ZA1> to <ZE6>;

<ZG4> a compound which is <ZF4> in conjunction with any one of <ZA1> to <ZE6>;

<ZH1> a compound wherein R^{EB} is a hydrogen atom;

<ZH2> a compound wherein R^{EB} is a methyl group;

<ZH3> a compound wherein R^{EB} is an ethyl group;

<ZI1> a compound which is <ZH1> in conjunction with any one of <ZA1> to <ZG4>;

<ZI2> a compound which is <ZH2> in conjunction with any one of <ZA1> to <ZG4>;

<ZI3> a compound which is <ZH3> in conjunction with any one of <ZA1> to <ZG4>;

<ZJ1> a compound wherein V¹⁹ is a divalent group obtained by removing two hydrogen atoms from [1,2,4]-oxadiazole;

<ZJ2> a compound wherein V^{1B} is a divalent group obtained by removing two hydrogen atoms [1,3,4]-oxadiazole;

<ZJ3> a compound wherein V^{1B} is a divalent group obtained by removing two hydrogen atoms [1,2,4]-thiadiazole;

<ZJ4> a compound wherein V^{1B} is a divalent group obtained by removing two hydrogen atoms [1,3,4]-thiadiazole;

<ZJ5> the compound according to <ZJ1>, wherein the positions at which the divalent group obtained by removing two hydrogen atoms from [1,2,4]-oxadiazole is bound to W^{B}-T^{B}-Z^{3B}-(CR^{6B}R^{7B})ₙ^{B}- and to —Ar^{B}-CH₂—NR^{1B}—B^{B}—CO₂R^{EB} are (5, 3);

<ZJ6> the compound according to <ZJ1>, wherein the positions at which the divalent group obtained by removing two hydrogen atoms from [1,2,4]-oxadiazole is bound to W^{B}-T^{B}-Z^{3B}-(CR^{6B}R^{7B})ₙ^{B} and to —Ar^{B}-CH₂—NR^{1B}—B^{B}—CO₂R^{EB} are (3, 5);

<ZJ7> the compound according to <ZJ3>, wherein the positions at which the divalent group obtained by removing two hydrogen atoms from [1,2,4]-thiadiazole is bound to W^{B}-T^{B}-Z^{3B}-(CR^{6B}R^{7B})ₙ^{B}- and to —Ar^{B}-CH₂—NR^{1B}—B^{B}—CO₂R^{EB} are (5, 3);

<ZJ8> the compound according to <ZJ3>, wherein the positions at which the divalent group obtained by removing two hydrogen atoms from [1,2,4]-thiadiazole is bound to W^{B}-T^{B}-Z^{3B}-(CR^{6B}R^{7B})ₙ^{B}- and to —Ar^{B}-CH₂—NR^{1B}—B^{B}—CO₂R^{EB} are (3, 5);

<ZK1> a compound which is <ZJ1> in conjunction with any one of <ZA1> to <ZI3>;

<ZK2> a compound which is <ZJ2> in conjunction with any one of <ZA1> to <ZI3>;

<ZK3> a compound which is <ZJ3> in conjunction with any one of <ZA1> to <ZI3>;

<ZK4> a compound which is <ZJ4> in conjunction with any one of <ZA1> to <ZI3>;

<ZK5> a compound which is <ZJ5> in conjunction with any one of <ZA1> to <ZI3>;

<ZK6> a compound which is <ZJ6> in conjunction with any one of <ZA1> to <ZI3>;

<ZK7> a compound which is <ZJ7> in conjunction with any one of <ZA1> to <ZI3>;

<ZK8> a compound which is <ZJ8> in conjunction with any one of <ZA1> to <ZI3>;

<ZL1> a compound wherein X^{4B} is a fluorine atom;

<ZL2> a compound wherein X^{4E} is a cyano group;

<ZL3> a compound wherein X^{4B} is a methyl group;

<ZL4> a compound wherein X^{4B} is a trifluoromethoxy group;

<ZM1> a compound which is <ZL1> in conjunction with any one of <ZA1> to <ZK8>;

<ZM2> a compound which is <ZL2> in conjunction with any one of <ZA1> to <ZK0>;

<ZM3> a compound which is <ZL3> in conjunction with any one of <ZA1> to <ZK8>;

<ZM4> a compound which is <ZL4> in conjunction with any one of <ZA1> to <ZK8>;

<ZN1> a compound wherein W^{B} is a C1-C6 alkyl group which may be substituted with 1 to 7 fluorine atoms;

<ZN2> a compound wherein W^{B} is a C5-C6 alkyl group which may be substituted with 1 to 7 fluorine atoms;

<ZN3> a compound wherein W^{B} is a C3-C7 cycloalkyl group which may be substituted with 1 to 7 fluorine atoms;

<ZN4> a compound wherein W^{B} is a C5-C7 cycloalkyl group which may be substituted with 1 to 7 fluorine atoms;

<ZN5> a compound wherein W^{B} is a monovalent group obtained by removing one hydrogen atom from benzene;

<ZN6> a compound wherein W^{B} is a monovalent group obtained by removing one hydrogen atom from naphthalene;

<ZN7> a compound wherein W^{B} is a monovalent group obtained by removing one hydrogen atom from furan;

<ZN8> a compound wherein W^{B} is a monovalent group obtained by removing one hydrogen atom from thiophene;

<ZN9> a compound wherein W^{B} is a monovalent group obtained by removing one hydrogen atom from pyrrole;

<ZN10> a compound wherein W^{B} is a monovalent group obtained by removing one hydrogen atom from oxazole;

<ZN11> a compound wherein W^{B} is a monovalent group obtained by removing one hydrogen atom from isoxazole;

<ZN12> a compound wherein W^{B} is a monovalent group obtained by removing one hydrogen atom from oxadiazole;

<ZN13> a compound wherein W^{B} is a monovalent group obtained by removing one hydrogen atom from thiazole;

<ZN14> a compound wherein W^{B} is a monovalent group obtained by removing one hydrogen atom from isothiazole;

<ZN15> a compound wherein W^{B} is a monovalent group obtained by removing one hydrogen atom from thiadiazole;

<ZN16> a compound wherein W^{B} is a monovalent group obtained by removing one hydrogen atom from imidazole;

<ZN17> a compound wherein W^{B} is a monovalent group obtained by removing one hydrogen atom from pyrazole;

<ZN18> a compound wherein W^{B} is a monovalent group obtained by removing one hydrogen atom from imidazole;

<ZN19> a compound wherein W^{B} is a monovalent group obtained by removing one hydrogen atom from pyrazole;

<ZN20> a compound wherein W^{B} is a monovalent group obtained by removing one hydrogen atom from pyran;

<ZN21> a compound wherein W^{B} is a monovalent group obtained by removing one hydrogen atom from pyridine;

<ZN22> a compound wherein W^{B} is a monovalent group obtained by removing one hydrogen atom from pyridazine;

<ZN23> a compound wherein W^{B} is a monovalent group obtained by removing one hydrogen atom from pyrimidine;

<ZN24> a compound wherein W^{B} is a monovalent group obtained by removing one hydrogen atom from pyrazine;

<ZN25> a compound wherein W^{B} is a monovalent group obtained by removing one hydrogen atom from oxazine;

<ZN26> a compound wherein W^{B} is a monovalent group obtained by removing one hydrogen atom from thiazine;

<ZO1> a compound which is <ZN1> in conjunction with any one of <ZA1> to <ZM4>;

<ZO2> a compound which is <ZN2> in conjunction with any one of <ZA1> to <ZM4>;

<ZO3> a compound which is <ZN3> in conjunction with any one of <ZA1> to <ZM4>;

<ZO4> a compound which is <ZN4> in conjunction with any one of <ZA1> to <ZM4>;

<ZO5> a compound which is <ZN5> in conjunction with any one of <ZA1> to <ZM4>;

<ZO6> a compound which is <ZN6> in conjunction with any one of <ZA1> to <ZM4>;

<ZO7> a compound which is <ZN7> in conjunction with any one of <ZA1> to <ZM4>;

<ZO8> a compound which is <ZN8> in conjunction with any one of <ZA1> to <ZM4>;

<ZO9> a compound which is <ZN9> in conjunction with any one of <ZA1> to <ZM4>;

<ZO10> a compound which is <ZN10> in conjunction with any one of <ZA1> to <ZM4>;

<ZO11> a compound which is <ZN11> in conjunction with any one of <ZA1> to <ZM4>;

<ZO12> a compound which is <ZN12> in conjunction with any one of <ZA1> to <ZM4>;

<ZO13> a compound which is <ZN13> in conjunction with any one of <ZA1> to <ZM4>;

<ZO14> a compound which is <ZN14> in conjunction with any one of <ZA1> to <ZM4>;

<ZO15> a compound which is <ZN15> in conjunction with any one of <ZA1> to <ZM4>;

<ZO16> a compound which is <ZN16> in conjunction with any one of <ZA1> to <ZM4>;

<ZO17> a compound which is <ZN17> in conjunction with any one of <ZA1> to <ZM4>;

<ZO18> a compound which is <ZN18> in conjunction with any one of <ZA1> to <ZM4>;

<ZO19> a compound which is <ZN19> in conjunction with any one of <ZA1> to <ZM4>;

<ZO20> a compound which is <ZN20> in conjunction with any one of <ZA1> to <ZM4>;

<ZO21> a compound which is <ZN21> in conjunction with any one of <ZA1> to <ZM4>;

<ZO22> a compound which is <ZN22> in conjunction with any one of <ZA1> to <ZM4>;

<ZO23> a compound which is <ZN23> in conjunction with any one of <ZA1> to <ZM4>;

<ZO24> a compound which is <ZN24> in conjunction with any one of <ZA1> to <ZM4>;

<ZO25> a compound which is <ZN25> in conjunction with any one of <ZA1> to <ZM4>;

<ZO26> a compound which is <ZN26> in conjunction with any one of <ZA1> to <ZM4>;

<ZP1> a compound wherein T^{B} is a single bond;

<ZP2> a compound wherein T^{B} is —O—;

<ZP3> a compound wherein T^{B} is —CONR^{TB}-;

<ZQ1> a compound which is <ZP1> in conjunction with any one of <ZA1> to <ZO26>;

<ZQ2> a compound which is <ZP2> in conjunction with any one of <ZA1> to <ZO26>;

<ZQ3> a compound which is <ZP3> in conjunction with any one of <ZA1> to <ZO26>;

<ZR1> a compound wherein X^{Z3B} is a fluorine atom;

<ZR2> a compound wherein X^{Z3B} is a cyano group;

<ZR3> a compound wherein X^{Z3B} is a trifluoromethoxy group;

<ZR4> a compound wherein X^{Z3B} is a methyl group;

<ZS1> a compound which is <ZR1> in conjunction with any one of <ZA1> to <ZQ3>;

<ZS2> a compound which is <ZR2> in conjunction with any one of <ZA1> to <ZQ3>;

<ZS3> a compound which is <ZR3> in conjunction with any one of <ZA1> to <ZQ3>;

<ZS4> a compound which is <ZR4> in conjunction with any one of <ZA1> to <ZQ3>;

<ZT1> a compound wherein Z^{3B} is a C5-C6 cycloalkylene group;

<ZT2> a compound wherein Z^{3B} is a divalent group obtained by removing two hydrogen atoms from benzene;

<ZT3> a compound wherein Z^{3B} is a divalent group obtained by removing two hydrogen atoms from thiophene;

<ZT4> a compound wherein Z^{3B} is a divalent group obtained by removing two hydrogen atoms from pyridine;

<ZU1> a compound which is <ZT1> in conjunction with any one of <ZA1> to <ZS4>;

<ZU2> a compound which is <ZT2> in conjunction with any one of <ZA1> to <ZS4>;

<ZU3> a compound which is <ZT3> in conjunction with any one of <ZA1> to <ZS4>;

<ZU3> a compound which is <ZT4> in conjunction with any one of <ZA1> to <ZS4>;

<ZV1> a compound wherein n^{B} is 0;

<ZV2> a compound wherein n^{B} is 1;

<ZW1> a compound which is <ZV1> in conjunction with any one of <ZA1> to <ZU3>; and

<ZW2> a compound which is <ZV2> in conjunction with any one of <ZA1> to <ZU3>;

Specifically, preferred examples among the compounds (1) of the present invention include the following compounds:

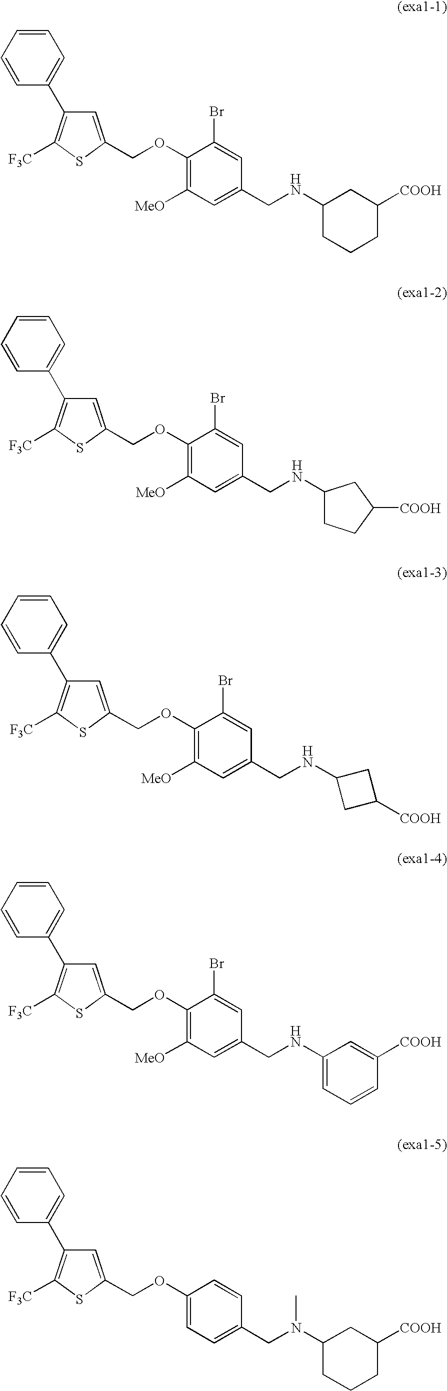

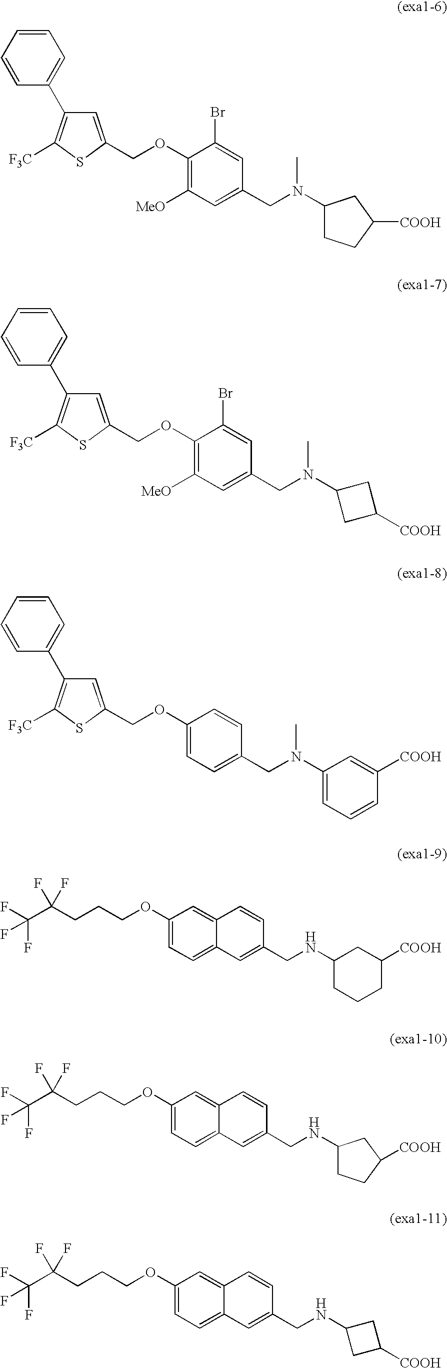

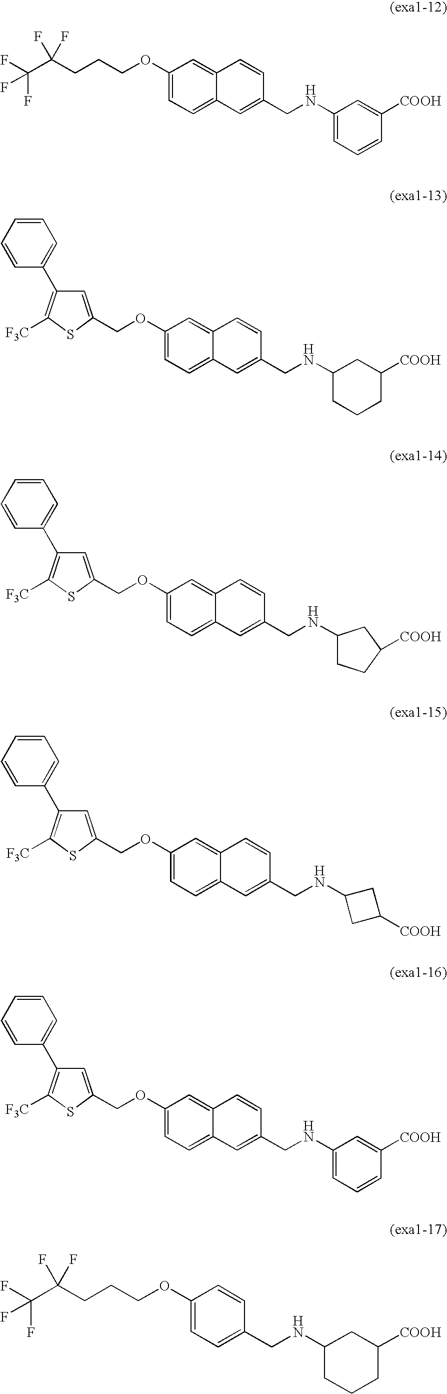

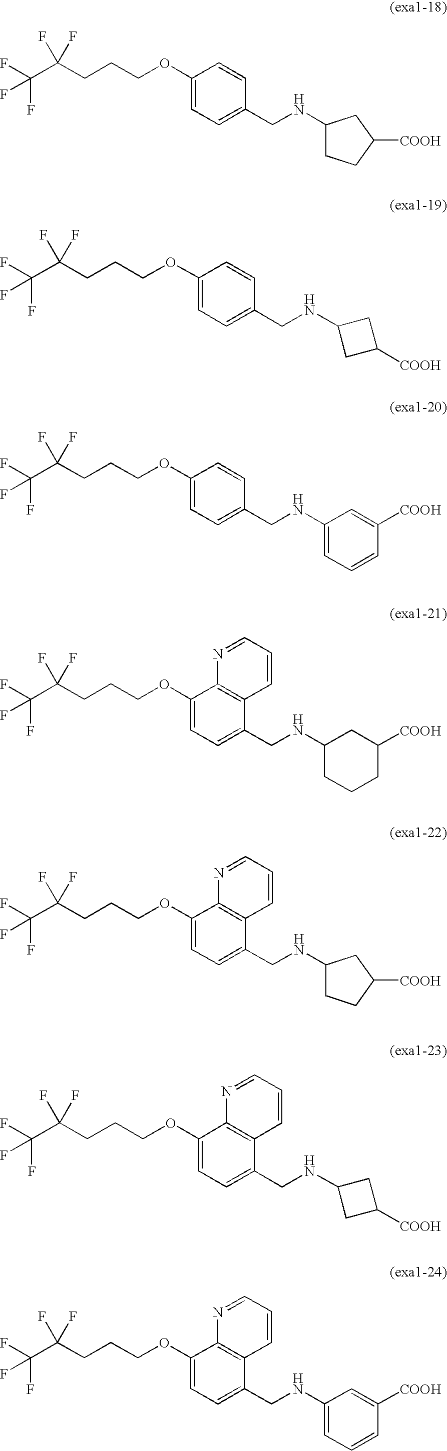

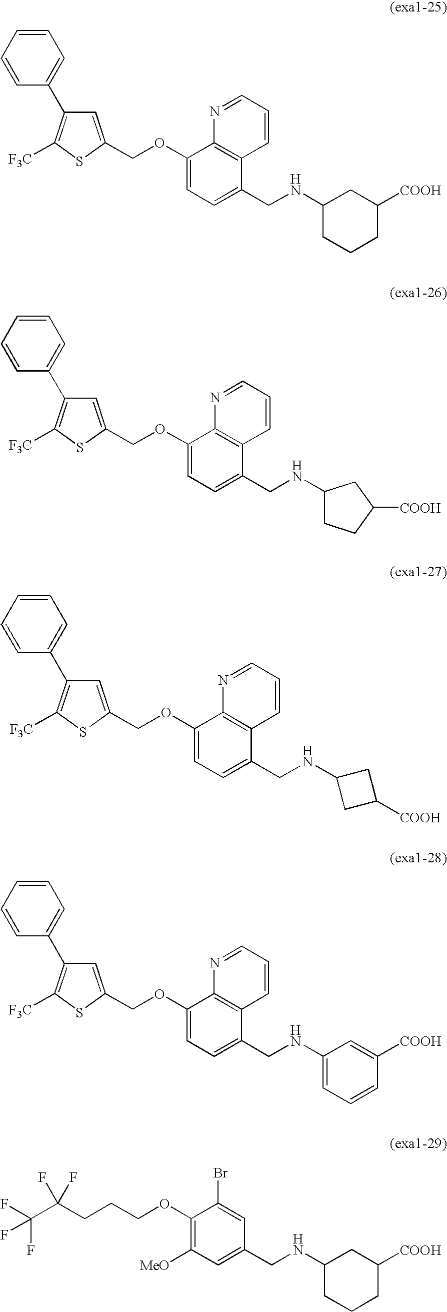

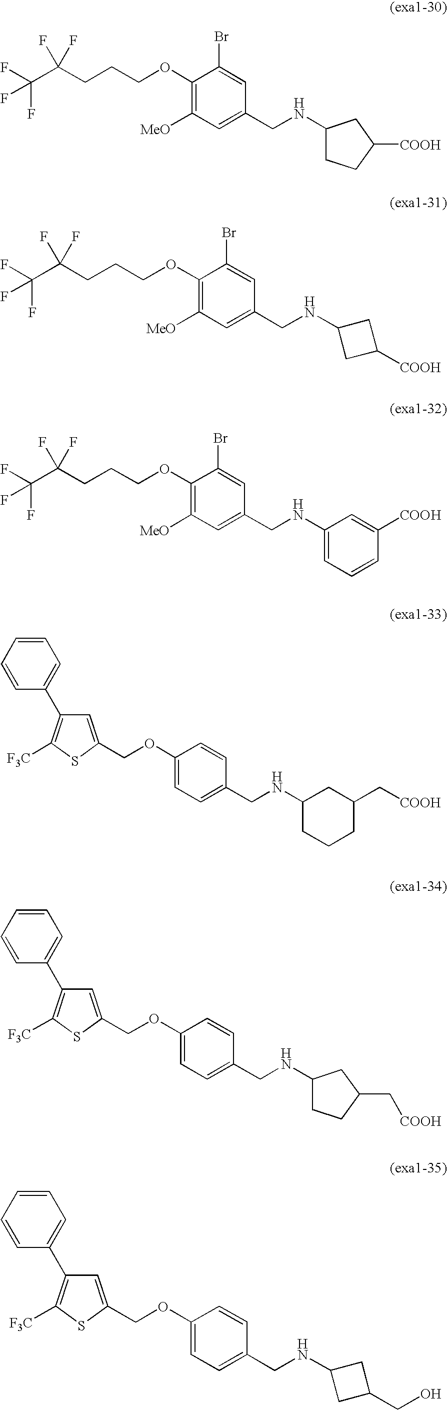

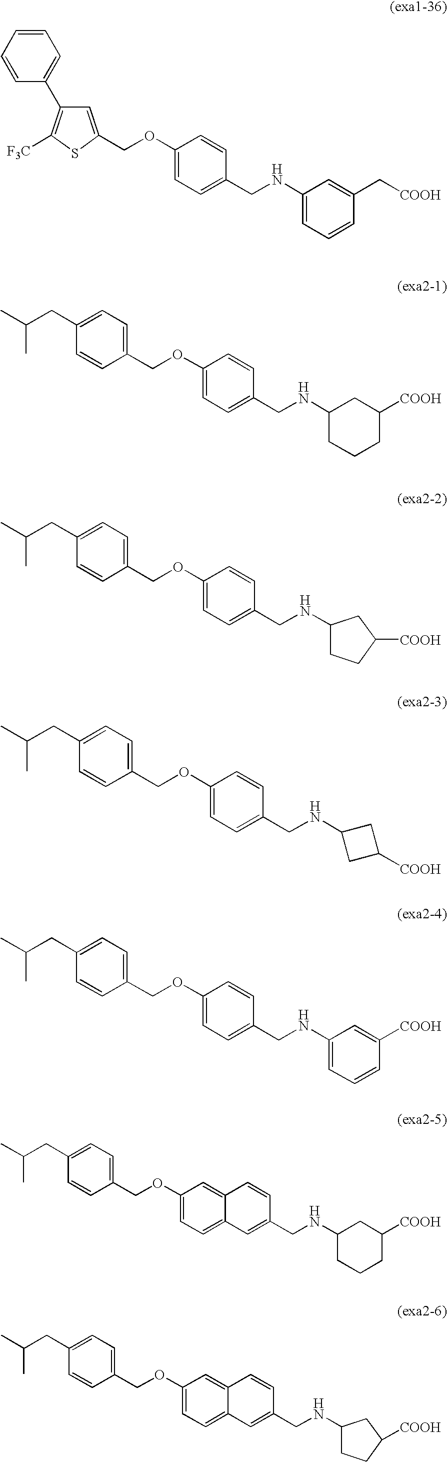

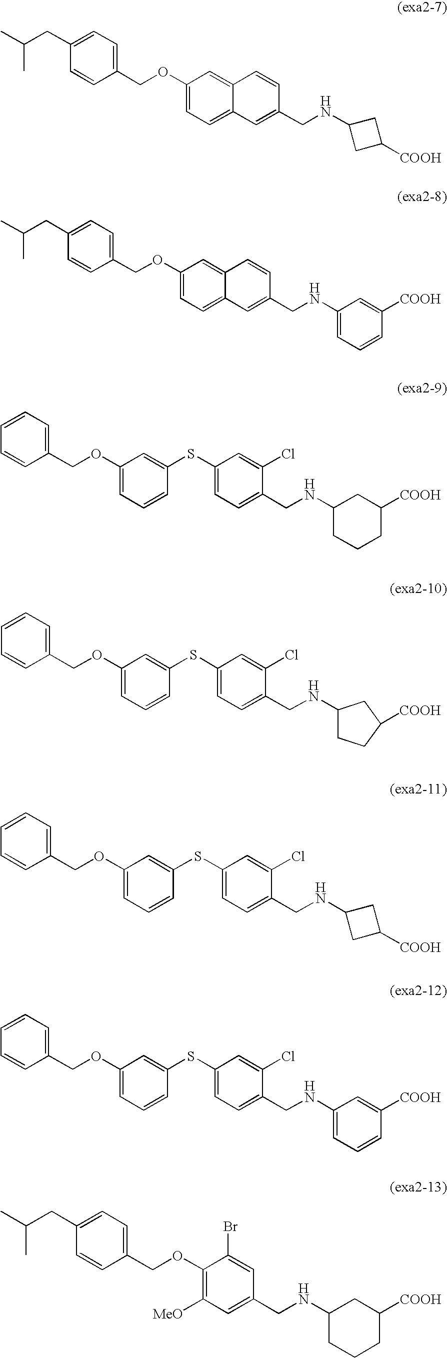

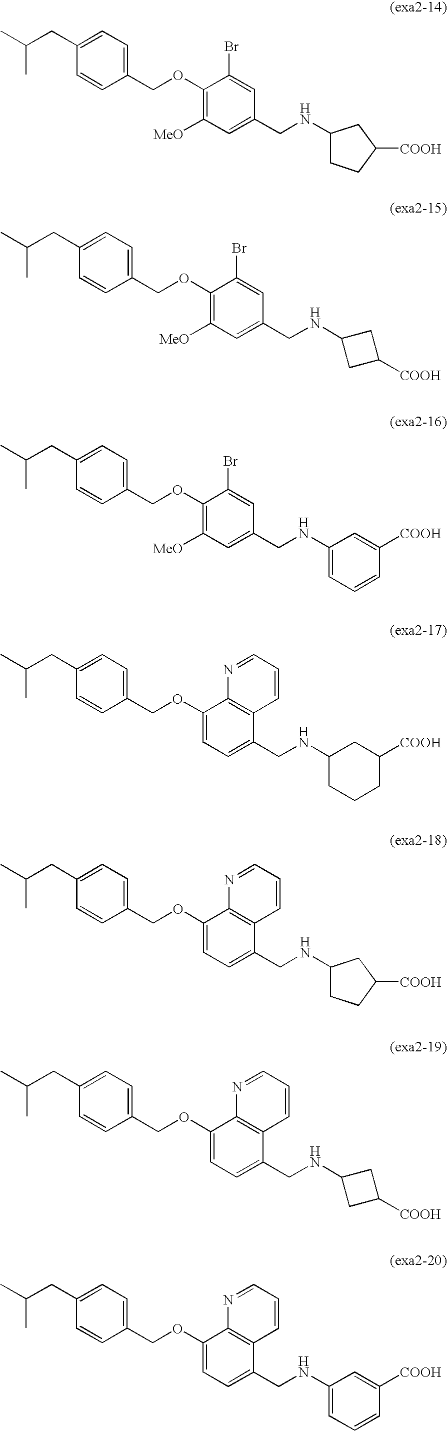

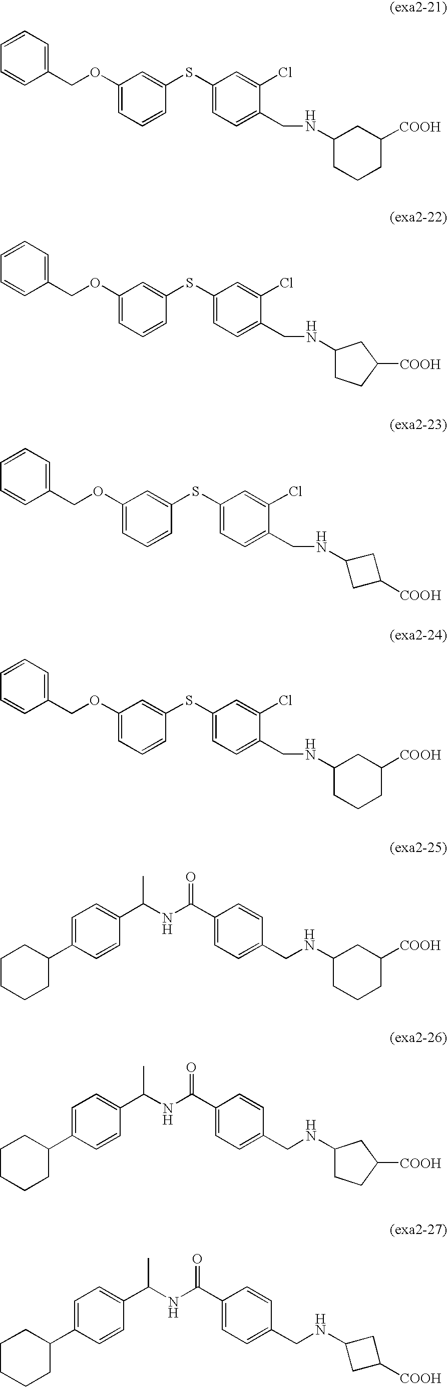

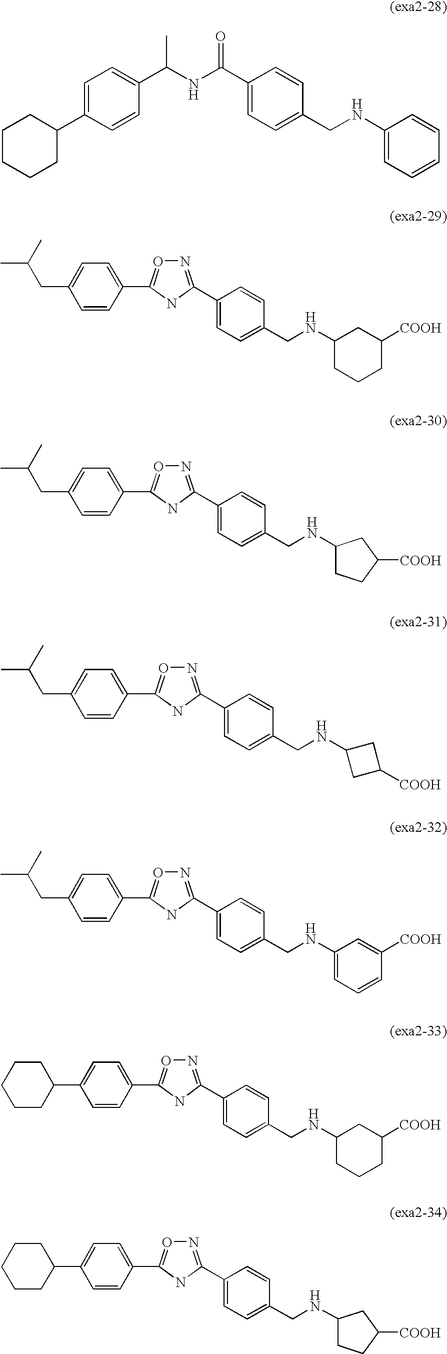

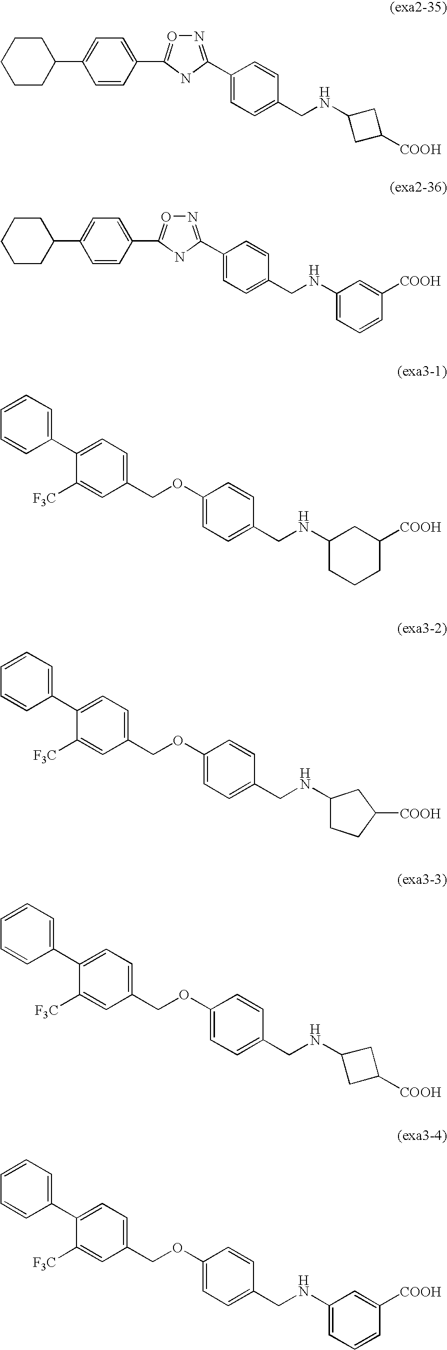

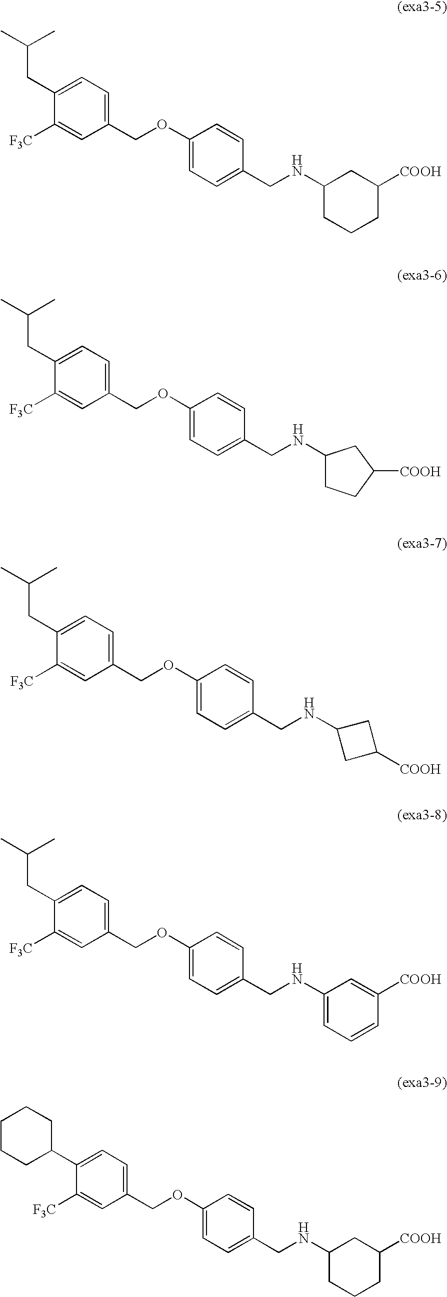

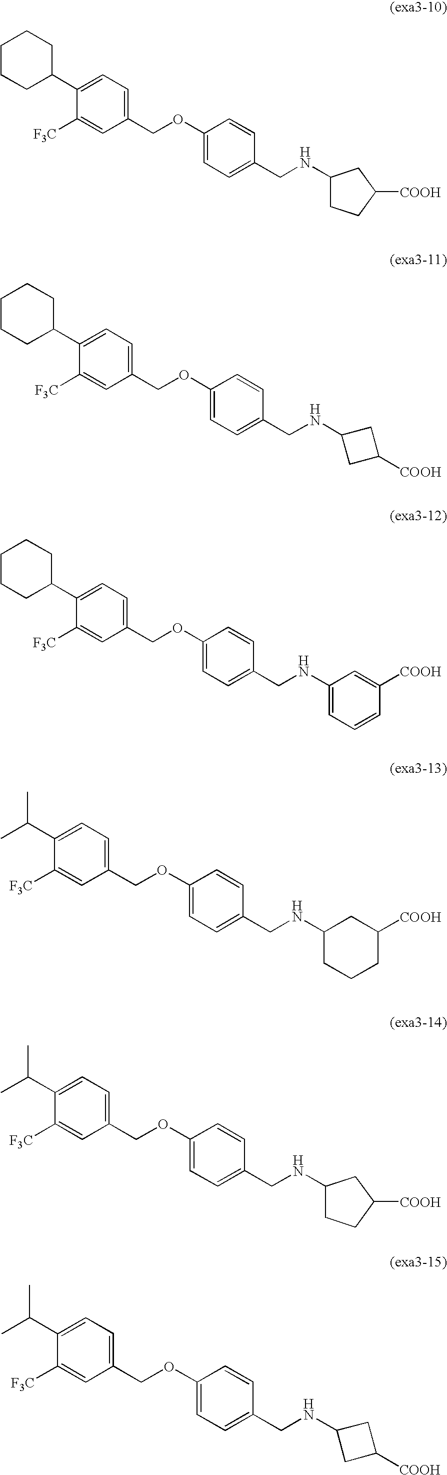

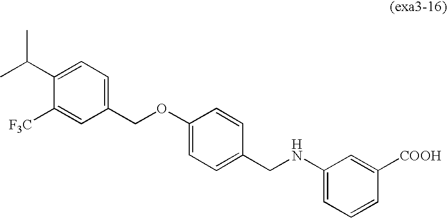

but the scope of the present invention is not to be limited to these preferred examples.

Furthermore, possible stereoisomers or racemic bodies of these compounds, or pharmacologically acceptable salts, hydrates, solvates of the compounds, the stereoisomers or the racemic bodies, or prodrugs of these compounds, stereoisomers, racemic bodies, salts, hydrates and solvates are also included in the scope of the present invention.

The compound represented by the general formula (2) of the present invention can be prepared using, for example, the reactions of the following general methods. Furthermore, the compound represented by the general formula (1) of the present invention can be prepared on the basis of the method for production of the compound represented by the general formula (2).

The compound of the present invention represented by the general formula (2) can be produced, for example, by the following methods, but the method for preparing the compound of the present invention is not to be particularly limited to the methods described below.

For each of the reactions, there is no particular limit to reaction time, but since the progress of reaction can be easily traced by means of known analysis techniques, it is desirable to terminate the reaction at a time point where the yield of the desired product obtained reaches the maximum.

The compound represented by the general formula (2) can be produced, for example, by the process as shown in the reaction scheme of Production Method A:

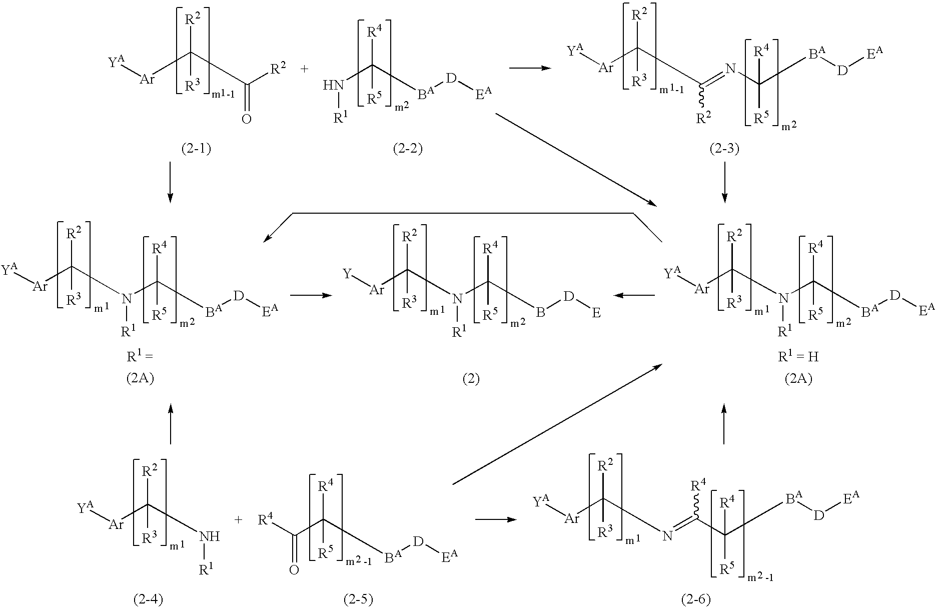

wherein R¹, R², R³, R⁴, R⁵, Ar, Y, B, D, E, m¹ and m² have the same meanings as the aforementioned; and Y^{A}, B^{A} and E^{A} have the same meanings as Y, B, and E aforementioned, respectively, or one or more groups among these may be protected. A compound represented by general formula (2A) can be produced by a reductive amination reaction between a compound represented by general formula (2-1) and a compound represented by general formula (2-2), or by a reductive amination reaction between a compound represented by general formula (2-4) and a compound represented by general formula (2-5).

The method for producing the compound represented by general formula (2A) by a reductive amination reaction can be carried out on the basis of the known methods for reduction amination reaction described in, for example, textbooks (the Fourth Series of Lectures on Experimental Chemistry, Vol. 20, Ch. 6, Maruzen) or literatures (Robert, M. B. et al., Tetrahedron Letters, 39, 3451 (1998)).

There is no particular limit to the type of the reducing agent used in the reductive amination reaction, but examples thereof include hydrogen, lithium aluminum hydride, sodium borohydride, sodium cyanoborohydride, borohydride triacetate, borane, and formic acid-triethylamine complexes. Preferred examples include hydrogen, sodium borohydride, sodium cyanoborohydride, borohydride triacetate, borane, or formic acid-triethylamine complexes.

The type of the solvent used in the reaction is not particularly limited as long as the solvent is inactive to the reduction reaction. However, examples thereof include alcohol solvents, saturated hydrocarbon solvents, halogenated hydrocarbon solvents, ether solvents, aromatic hydrocarbon solvents, N,N-dimethylformamide, or dimethylsulfoxide. These solvents may also be used individually or as solvent mixtures at any ratio. The alcohol solvent may be exemplified by methanol, ethanol, 2-propanol or the like; the saturated hydrocarbon solvent may be exemplified by pentane, hexane, heptane, cyclohexane or the like; and the halogenated hydrocarbon solvent may be exemplified by dichloromethane, chloroform, 1,2-dichloroethane or the like. The ether solvent may be exemplified by tetrahydrofuran, diethyl ether, 1,4-dioxane or the like; and the aromatic hydrocarbon solvent may be exemplified by toluene, xylene or the like. Preferred examples include 2-propanol, dichloromethane, tetrahydrofuran, toluene, N,N-dimethylformamide and the like.

The amount of the reducing agent is preferably 0.1 moles or more, and more preferably an equimolar or more amount, relative to the compound represented by general formula (2-1) or general formula (2-4). Furthermore, an amount of 100-fold or less the molar amount of the compound is preferable, and an amount of 10-fold or less the molar amount of the compound is more preferable. The reaction temperature is not particularly limited, but it is preferable to perform the reaction at −20° C. or higher, and more preferably at 0° C. or higher.

The reaction time may vary depending on the raw material compound, solvent, reaction temperature or the like, but is usually 30 minutes to 72 hours, and preferably 1 hour to 48 hours.

Furthermore, when the reductive amination reaction is performed using a compound represented by general formula (2-2) or by general formula (2-4) wherein R¹ is a hydrogen atom, the reduction reaction can be performed usually without isolating the compound represented by general formula (2-3) or by general formula (2-6), which is generated as an intermediate. However, if the intermediate can be isolated, the compound may be isolated and then supplied to the reduction reaction.

Also, among the compounds represented by the general formula (2A), a compound in which R¹ is a C1-4 alkyl group can also be produced by a reductive amination reaction between a compound represented by the general formula (2A) in which R¹ is a hydrogen atom, and a known C1-4 saturated aldehyde or a C1-4 saturated ketone. For the reductive amination reaction, the methods as described above may be listed for example.

Furthermore, as another method for the process described above, a compound represented by general formula (2A) in which R¹ is a C1-4 alkyl group, can also be produced by an alkylation reaction between a compound represented by general formula (2A) in which R¹ is a hydrogen atom, and a known compound represented by R¹L¹ (wherein R¹ represents a C1-4 alkyl group, and L¹ represents a leaving group). For the alkylation reaction, a base may be present according to necessity.

The leaving group, L¹, may be exemplified by a halogen atom, an acyloxy group, or the like. The halogen atom is preferably a chlorine atom, a bromine atom, or an iodine atom. As the acyloxy group, an alkylsulfonyloxy group which may be halogenated, an arylsulfonyloxy group which may be substituted, an alkyloxy sulfonyloxy group, or the like is preferred. The alkylsulfonyloxy group which may be halogenated is preferably a methanesulfonyloxy group, a trifluoromethanesulfonyloxy group, or the like. The arylsulfonyloxy group which may be substituted is preferably a benzenesulfonyloxy group, a para-toluenesulfonyloxy group, or the like. The alkyloxysulfonyloxy group is preferably a methoxysulfonyloxy group, an ethoxysulfonyloxy group, or the like.

With regard to the alkylation reaction, the amount of use of the compound represented by R¹L¹ is typically 0.9- to 10-fold the molar amount, and preferably 1- to 3-fold the molar amount, of the compound represented by general formula (2A) in which R¹ is a hydrogen atom. Examples of the inactive solvent used herein include halogenated hydrocarbons such as dichloromethane or chloroform; ethers such as tetrahydrofuran, dioxane or diethyl ether; dimethylsulfoxide, N,N-dimethylformamide, and acetonitrile. These can be used individually or as solvent mixtures. Examples of the base used in the reaction described above include alkali metal compounds such as sodium hydrogen carbonate, sodium hydroxide, sodium hydride, potassium carbonate, sodium carbonate, potassium hydroxide or sodium methylate; and organic tertiary amines such as pyridine, trimethylamine, triethylamine, N,N-diisopropylethylamine or N-methylmorpholine. The amount of use thereof is, for example, typically 1- to 20-fold the molar amount, and preferably 1- to 10-fold the molar amount, of the compound represented by general formula (2A) in which R¹ is a hydrogen atom. The reaction temperature is preferably −30° C. or higher, and more preferably 0° C. or higher. Also, 150° C. or lower is preferable, and 120° C. or lower is more preferable.

The reaction time may vary depending on the raw material compound, base, solvent, reaction temperature, or the like, but for example, the reaction time is typically from 30 minutes to 72 hours, and preferably from 1 hour to 48 hours.

The compound represented by general formula (2) can be produced, if one or more of protective groups are present on the compound represented by general formula (2A), by deprotecting all of the protective groups simultaneously or sequentially. The deprotection reaction may be favorably performed on the basis of known methods, for example, the methods described in Protective Groups in Organic Synthesis, published by John Wiley and Sons (1999). If there is no protective group present on the compound represented by general formula (2A), it will be easily understood by those having ordinary skill in the art that the compound represented by general formula (2A) corresponds to the compound represented by general formula (2).

Furthermore, in another method, the compound represented by general formula (2A) can be produced, for example, as shown in the reaction scheme of Production Method B:

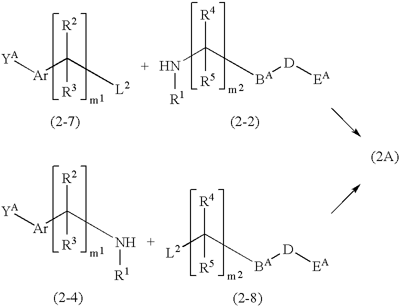

wherein R¹, R², R³, R⁴, R⁵, Ar, D, m¹ and m² have the same meanings as the defined above; and Y^{A}, B^{A} and E^{A} have the same meanings as Y, B and E described above, respectively, or one or more groups among these may be protected, L² represents a leaving group.

through an alkylation reaction between a compound represented by general formula (2-7) and a compound represented by general formula (2-2), or through an alkylation reaction between a compound represented by general formula (2-4) and a compound represented by general formula (2-8). For the alkylation reaction, a base may be present according to necessity. With regard to the compounds represented by general formula (2-7) and general formula (2-8), the leaving group, L², may be exemplified by a halogen atom, an acyloxy group, or the like. The halogen atom is preferably a chlorine atom, a bromine atom, or an iodine atom, while the acyloxy group is preferably an alkylsulfonyloxy group which may be halogenated, an arylsulfonyloxy group which may be substituted, or the like. The alkylsulfonyloxy group which may be halogenated is preferably a methanesulfonyloxy group, a trifluoromethanesulfonyloxy group, or the like. The arylsulfonyloxy group which may be substituted is preferably a benzenesulfonyloxy group, a para-toluenesulfonyloxy group, or the like. For the alkylation reaction, the methods as described above may be listed for example.

In the reaction scheme for the Production Method A described above, the compound represented by general formula (2-1) can be produced, for example, by the method shown in the reaction scheme for Production Method C:

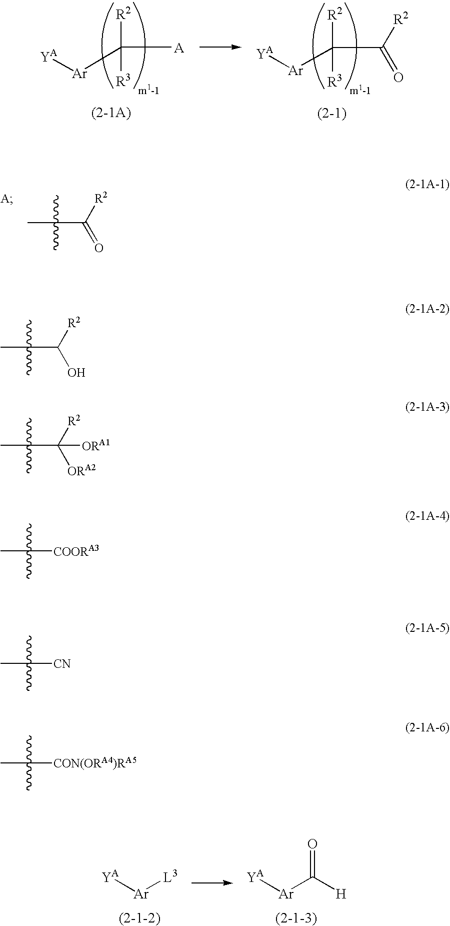

wherein , R², R³, Ar, Y^{A} and m¹ have the same meanings as the defined above; A represents the group (2-1A-1), group (2-1A-2), group (2-1A-3), group (21A-4) r group (2-1A-5), or group (2-1A-6) as illustrated in the reaction scheme for Production Method C; R^{A1} and R^{A2}, which may be the same or different, each represent a C1-4 alkyl group, or R^{A1} and R^{A2} may be joined to form a C2-C5 alkylene group; R^{A3} represents a C1-4 alkyl group; R^{A4} and R^{A5}, which may be the same or different, each represent a C1-4 alkyl group; and L³ represents a leaving group.

For R^{A1} and R^{A2}, the C1-4 alkyl group may be exemplified by a methyl group, an ethyl group, or the like; and the C2-C5 alkylene group may be exemplified by an ethylene group, a propylene group, or the like. The leaving group, L³, may be exemplified by a chlorine atom, a bromine atom, or an iodine atom.

The compound represented by the general formula (2-1) can be produced by converting a compound represented by general formula (2-1A) by a functionality conversion reaction. In addition, it is easily understood by those having ordinary skill in the art that the compound represented by general formula (2-1A-1) corresponds to the compound represented by general formula (2-1).

A significant number of the compounds represented by general formula (2-1A) in which A is the group (2-1A-2), the group (2-1A-4) or the group (2-1A-5), are commercially available, and a person ordinarily skilled in the art can produce the compound represented by the general formula (2-1) using known methods.

A compound represented by general formula (2-1) in which the terminal R² is a hydrogen atom, can be produced, for example, by reducing a compound represented by general formula (2-1A) in which A is the group (2-1A-4), the group (2-1A-5) or the group (2-1A-6), with a metal hydride, a metal-hydrogen complex compound or the like.

The reducing agent used in the reduction reaction of the compound represented by general formula (2-1A) in which A is the group (2-1A-4), is preferably a metal hydride, and the metal hydride is preferably diisobutylaluminum hydride, or the like.

The reducing agent used in the reduction reaction of the compound represented by general formula (2-1A) in which A is the group (2-1A-6), is preferably a metal-hydrogen complex compound, and the metal-hydrogen complex compound is preferably lithium aluminum hydride, or the like.

The type of the solvent used in the reduction reaction of the compound represented by general formula (2-1A) in which A is the group (2-1A-4) or the group (2-1A-6), is not particularly limited as long as the solvent is inactive to the reduction reaction. Examples thereof include saturated hydrocarbon solvents, halogenated hydrocarbon solvents, ether solvents, and aromatic hydrocarbon solvents. These solvents may be used individually or as solvent mixtures at any ratio. The saturated hydrocarbon solvent may be exemplified by pentane, hexane, heptane, cyclohexane, or the like; and the halogenated hydrocarbon solvent may be exemplified by dichloromethane, chloroform, 1,2-dichloroethane, or the like. The ether solvent may be exemplified by tetrahydrofuran, diethyl ether, 1,4-dioxane, or the like; and the aromatic hydrocarbon solvent may be exemplified by toluene, xylene, or the like. Preferred examples include diethyl ether, tetrahydrofuran, toluene, or solvent mixtures of these solvents at any ratio.

The amount of the reducing agent is preferably 0.1-fold or more the molar amount, and more preferably an equimolar or more amount, relative to the compound represented by general formula (2-1A) in which A is the group (2-1A-4) or the group (2-1A-6). Also, the amount is preferably 100-fold or less the molar amount of the compound, and more preferably 10-fold or less the molar amount of the compound.

The reaction temperature may vary depending on the raw material compound, reducing agent, solvent or the like, but it is preferable to perform the reaction typically at −100° C. or higher, and it is also preferable to perform the reaction at 50° C. or lower.

The reaction time may vary depending on the raw material compound, reducing agent, solvent, reaction temperature or the like, but the time may be typically from 5 minutes to 12 hours, for example.

A compound represented by general formula (2-1) in which the terminal R² is a C1-4 alkyl group, can be produced, for example, through a reaction between a compound represented by general formula (2-1A) in which A is the group (2-1A-5) or the group (2-1A-6), and an organometallic reagent represented by R²M (wherein R² represents a C1-4 alkyl group; and M represents a metal atom or a halogenated metal atom).

The metal atom or halogenated metal atom, M, may be exemplified by an alkali metal atom, or a halogenated alkaline earth metal atom. The alkali metal atom is preferably Li, while the halogenated alkaline earth metal atom is preferably MgCl, MgBr, or MgI.

The type of the solvent used in the reaction is not particularly limited as long as the solvent is inactive to the reaction. Examples thereof include saturated hydrocarbon solvents, aromatic hydrocarbon solvents, and ether solvents. The saturated hydrocarbon solvent may be exemplified by pentane, hexane, heptane, cyclohexane, or the like; the aromatic hydrocarbon solvent may be exemplified by toluene, xylene, or the like; the ether solvent may be exemplified by tetrahydrofuran, diethyl ether, 1,2-dimethoxyethane, 1,4-dioxane, or the like. Hexane, cyclohexane, toluene, tetrahydrofuran, diethyl ether, or solvent mixtures of these solvents at any ratio are preferred.

The amount of the organometallic reagent represented by R²M, which is used in the reaction, is preferably 0.1-fold or more the molar amount, and more preferably an equimolar or more amount, relative to the compound represented by general formula (2-1A-5) or general formula (2-1A) in which A is the group (2-1A-6), Also, the amount is preferably 100-fold or less the molar amount, and more preferably 10-fold or less the molar amount.

The reaction temperature may vary depending on the raw material compound, solvent or the like, but typically, it is preferable to perform the reaction at −50° C. or higher, and it is preferable to perform the reaction at 100° C. or lower.

The reaction time may vary depending on the raw material compound, solvent, reaction temperature or the like, but the time may be typically from 10 minutes to 24 hours, for example.

The compound represented by general formula (2-1) can also be produced, for example, by oxidizing a compound represented by general formula (2-1A) in which A is the group (2-1A-2).

A significant number of the compounds represented by general formula (2-1A) in which A is the group (2-1A-2), are commercially available, and a person ordinarily skill in the art can produce the compound represented by general formula (2-1) using known oxidation reactions.

The oxidation reaction may be exemplified by the oxidation reaction using the Dess-Martin reagent.

The solvent used in the oxidation reaction is not particularly limited as long as the solvent is inactive to the oxidation reaction. The solvent is, for example, dichloromethane.

The amount of the oxidizing agent used in the oxidation reaction is preferably 0.1-fold or more the molar amount, and more preferably an equimolar or more amount, relative to the compound represented by general formula (2-1A) in which A is the group (2-1A-2). Also, the amount is preferably 100-fold or less the molar amount of the compound, and more preferably 10-fold or less the molar amount of the compound.

The reaction temperature may vary depending on the raw material compound, solvent or the like, but typically, it is preferable to perform the reaction at −50° C. or higher, and it is preferable to perform the reaction at 50° C. or lower.

The reaction time may vary depending on the raw material compound, solvent, reaction temperature or the like, but the time may be typically from 1 hour to 24 hours, for example.

Furthermore, the compound represented by general formula (2-1) can be produced by deprotecting a compound represented by general formula (2-1A) in which A is the group (2-1A-3) in the presence of an acid catalyst.

The acid catalyst used in the deprotection reaction is not particularly limited, but is, for example, para-toluenesulfonic acid.

The solvent used in the deprotection reaction is not particularly limited, but is, for example, water, acetone, or solvent mixtures of these solvents at any ratio.

The amount of the acid catalyst used in the deprotection reaction is preferably 0.001-fold or more the molar amount of the compound represented by general formula (2-1A) in which A is the group (2-1A-3).

The reaction temperature may vary depending on the raw material compound, solvent or the like, but typically, it is preferable to perform the reaction at ambient temperature.

The reaction time may vary depending on the raw material compound, solvent, reaction temperature or the like, but the time may be typically from 1 hour to 48 hours, for example.

A compound represented by general formula (2-1-3), which is a compound represented by general formula (2-1) in which R² is a hydrogen atom and m¹ is 1, can be produced by treating a solution of an organometallic compound that can be obtained by metal-halogen exchange between a compound represented by formula (2-1-2) and an organolithium compound, with N,N-dimethylformamide to perform a formylation reaction.

The organolithium compound used in the metal-halogen exchange reaction may be exemplified by n-butyllithium, sec-butyllithium, tert-butyllithium, or the like.

The type of the solvent used in the metal-halogen exchange reaction is not particularly limited as long as the solvent is inactive to the reaction. Examples thereof include saturated hydrocarbon solvents, aromatic hydrocarbon solvents, and ether solvents. The saturated hydrocarbon solvent may be exemplified by pentane, hexane, heptane, cyclohexane, or the like; the aromatic hydrocarbon solvent may be exemplified by toluene, xylene, or the like; and the ether solvent may be exemplified by tetrahydrofuran, diethyl ether, 1,2-dimethoxyethane, 1,4-dioxane, or the like. Pentane, hexane, cyclohexane, toluene, tetrahydrofuran, 1,2-dimethoxyethane, diethyl ether, or solvent mixtures of these solvents at any ratio are preferred.

The amount of the organometallic reagent used in the metal-halogen exchange reaction is preferably 0.1-fold or more the molar amount, and more preferably 0.5-fold or more the molar amount, of the compound represented by general formula (2-1-2). Also, the amount is preferably 10-fold or less the molar amount, and more preferably 5-fold or less the molar amount, of the compound.

For the metal-halogen exchange reaction, the reaction temperature may vary depending on the raw material compound, solvent or the like, but typically, it is preferable to perform the reaction at −100° C. or higher, and it is also preferable to perform the reaction at 0° C. or lower. The reaction time may vary depending on the raw material compound, solvent, reaction temperature or the like, but the time may be typically from 5 minutes to 12 hours, for example.

The amount of N,N-dimethylformamide used in the formylation reaction is preferably 0.1-fold or more the molar amount, and more preferably 0.5-fold or more the molar amount, of the compound represented by general formula (2-1-2). Also, the amount is preferably 10-fold or less the molar amount, and more preferably 5-fold or less the molar amount, of the compound.

For the formylation reaction, the reaction temperature may vary depending on the raw material compound, solvent or the like, but typically, it is preferable to perform the reaction at −100° C. or higher, and it is also preferable to perform the reaction at 50° C. or lower. The reaction time may vary depending on the raw material compound, solvent, reaction temperature or the like, but the time may be typically from 5 minutes to 12 hours, for example.

In the reaction scheme for the Production Method B aforementioned, compounds represented by general formulas (2-7) and (2-4) can be produced, for example, by the method shown in the reaction scheme for Production Method D:

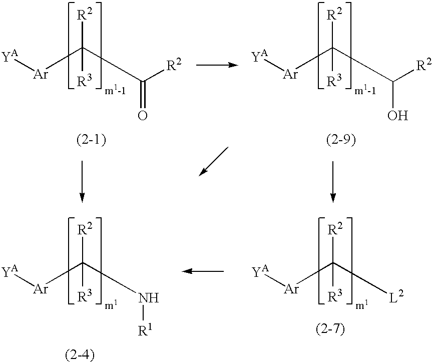

wherein R¹, R², R³, Ar, Y^{A}, m¹ and L² have the same meanings as the defined above, with the proviso that among the groups R³, the group R³ substituting the methylene group which is directly bound to L², and the group R³ substituting the methylene group which is directly bound to the nitrogen atom bound to R¹, each represent a hydrogen atom.

A compound represented by general formula (2-9) can be produced by reducing the compound represented by general formula (2-1).

The type of the reducing agent used in the reduction reaction is not particularly limited, but the reducing agent is, for example, a metal-hydrogen complex compound. The metal-hydrogen complex compound may be exemplified by sodium borohydride or the like.

The type of the solvent used in the reaction is not particularly limited as long as the solvent is inactive to the reduction reaction, but the solvent is, for example, alcohol solvent. The alcohol solvent may be exemplified by methanol, ethanol, or the like.

The amount of the reducing agent is preferably 0.1-fold or more the molar amount, and more preferably an equimolar or more amount, of the compound represented by general formula (2-1).

Also, the amount is preferably 100-fold or less the molar amount, and more preferably 10-fold or less the molar amount, of the compound.

The reaction temperature may vary depending on the raw material compound, reducing agent, solvent or the like, but is typically from −20° C. to room temperature.

The reaction time may vary depending on the raw material compound, reducing agent, solvent, reaction temperature or the like, but the time may be typically from 1 minute to 2 hours, for example.

A compound represented by general formula (2-7) can be produced by converting the hydroxyl group of a compound represented by general formula (2-9) to L².

With regard to the compound represented by general formula (2-7), when L² is an acyloxy group, the compound represented by general formula (2-7) can be produced, for example, by treating the compound represented by general formula (2-9) with a corresponding acyl halide in an inactive solvent, in the presence of a base. The acyl halide may be exemplified by para-toluenesulfonyl chloride, methanesulfonyl chloride, or the like.

The base used in the acylation reaction may be exemplified by triethylamine, diisopropylethylamine, pyridine, or the like.

The type of the solvent used in the acylation reaction is not particularly limited as long as the solvent is inactive to the acylation reaction. Examples thereof include saturated hydrocarbon solvents, halogenated hydrocarbon solvents, ether solvents, and aromatic hydrocarbon solvents. These solvents may be used individually or as solvent mixtures at any ratio. The saturated hydrocarbon solvent may be exemplified by pentane, hexane, heptane, cyclohexane, or the like; and the halogenated hydrocarbon solvent may be exemplified by dichloromethane, chloroform, 1,2-dichloroethane, or the like. The ether solvent may be exemplified by tetrahydrofuran, diethyl ether, or 1,4-dioxane; and the aromatic hydrocarbon solvent may be exemplified by toluene, xylene, or the like. Preferred examples include dichloromethane, chloroform, diethyl ether, tetrahydrofuran, toluene, and the like.

The amount of the acyl halide used in the acylation reaction is preferably 0.5-fold or more the molar amount, and more preferably an equimolar or more amount, of the compound represented by general formula (2-9). Also, the amount is preferably 10-fold or less the molar amount, and more preferably 2-fold or less the molar amount, of the compound.

The amount of the base used in the acylation reaction is preferably an equimolar or more amount of the acyl halide, and also preferably 2-fold or less the molar amount of the acylhalide.

The reaction temperature may vary depending on the raw material compound, solvent or the like, but typically, it is preferable to perform the reaction at a temperature within the range of −30° C. to room temperature.

The reaction time may vary depending on the raw material compound, solvent, reaction temperature or the like, but the time may be typically from 1 minute to 12 hours, for example.

With regard to the compound represented by general formula (2-7), when L² is a bromine atom, the compound represented by general formula (2-7) can be produced, for example, by treating the compound represented by general formula (2-9) with carbon tetrabromide in an inactive solvent in the presence of triphenylphosphine.

The type of the solvent used in the halogenation reaction is not particularly limited as long as the solvent is inactive to the halogenation reaction. Examples thereof include saturated hydrocarbon solvents, halogenated hydrocarbon solvents, ether solvents, and aromatic hydrocarbon solvents. These solvents may be used individually or as solvent mixtures at any ratio. The saturated hydrocarbon solvent may be exemplified by pentane, hexane, heptane, or cyclohexane; and the halogenated hydrocarbon solvent may be exemplified by dichloromethane, chloroform, or 1,2-dichloroethane. The ether solvent may be exemplified by tetrahydrofuran, diethyl ether, or 1,4-dioxane; and the aromatic hydrocarbon solvent may be exemplified by toluene, xylene, or the like. Preferred examples include dichloromethane, chloroform, diethyl ether, tetrahydrofuran, toluene, and the like.

The amount of carbon tetrabromide used in the halogenation reaction is preferably 0.5-fold or more the molar amount, and more preferably an equimolar or more amount, of the compound represented by general formula (2-9). Also, the amount is preferably 10-fold or less the molar amount, and more preferably 5-fold or less the molar amount, of the compound.

The amount of triphenylphosphine used in the halogenation reaction is preferably an equimolar or more amount, and also 5-fold or less the molar amount, of the carbon tetrabromide.

The reaction temperature may vary depending on the raw material compound, solvent or the like, but typically, it is preferable to perform the reaction at −30° C. or higher, and it is also preferable to perform the reaction at 50° C. or lower.

The reaction time may vary depending on the raw material compound, solvent, reaction temperature or the like, but the time may be typically from 1 minute to 12 hours, for example.

The compound represented by general formula (2-4) can be produced through an alkylation reaction between the compound represented by general formula (2-7), and a compound represented by general formula R¹NH₂ (wherein R¹ represents a C1-4 alkyl group).

For the alkylation reaction, for example, the methods as described above may be listed.

Furthermore, in another method, the compound represented by general formula (2-4) can be produced through a reductive amination reaction between the compound represented by general formula (2-1) and a compound represented by general formula R¹NH₂.

For the reductive amination reaction, for example, the methods as described above may be listed.

Also, the compound represented by general formula (2-4) can be produced by performing a Mitsunobu reaction using a compound represented by general formula (2-9) and an imide compound, and then performing a deprotection reaction.

The imide compound used in the Mitsunobu reaction may be exemplified by phthalimide, nitrophthalimide, or the like.

The amount of use of the imide compound used in the Mitsunobu reaction is preferably 0.5-fold or more the molar amount, and more preferably 1-fold or more the molar amount, of the compound represented by general formula (2-9). Also, the amount is preferably 20-fold or less the molar amount, and more preferably 10-fold or less the molar amount, of the compound.

The azo compound used in the Mitsunobu reaction may be exemplified by ethyl azodicarboxylate, diisopropyl azodicarboxylate, N,N,N′,N′-tetramethylazodicarboxamide, N,N,N′,N′-tetraisopropylazodicarboxamide, or the like.

The amount of use of the azo compound used in the Mitsunobu reaction is preferably 0.5-fold or more the molar amount, and more preferably 1-fold or more the molar amount, of the compound represented by general formula (2-9). Also, the amount is preferably 20-fold or less the molar amount, and more preferably 10-fold or less the molar amount, of the compound.

The phosphine reagent used in the Mitsunobu reaction may be exemplified by triphenylphosphine, tri-n-butylphosphine, or the like.

The amount of use of the phosphine reagent used in the Mitsunobu reaction is preferably 0.5-fold or more the molar amount, and more preferably 1-fold or more the molar amount, of the compound represented by general formula (2-9). Also, the amount is preferably 20-fold or less the molar amount, and molar preferably 10-molar or less the molar amount, of the compound.

The type of the solvent used in the Mitsunobu reaction is not particularly limited as long as the solvent is inactive to the reaction. Examples thereof include saturated hydrocarbon solvents, halogenated hydrocarbon solvents, ether solvents, and aromatic hydrocarbon solvents, and also these solvents may be used individually or as solvent mixtures at any ratio. The saturated hydrocarbon solvent may be exemplified by pentane, hexane, heptane, cyclohexane, or the like; and the halogenated hydrocarbon solvent may be exemplified by dichloromethane, chloroform, 1,2-dichloroethane, or the like. The ether solvent may be exemplified by tetrahydrofuran, diethyl ether, 1,4-dioxane, or the like; and the aromatic hydrocarbon solvent may be exemplified by toluene, xylene, or the like. Preferred examples include hexane, dichloromethane, chloroform, tetrahydrofuran, diethyl ether, toluene, or solvent mixtures of these solvents at any ratio.

For the Mitsunobu reaction, the reaction temperature is preferably −50° C. or higher, and more preferably −30° C. or higher. Also, the temperature is preferably the boiling point of the solvent used in the reaction or below, and more preferably 30° C. or lower.

For the deprotection reaction, hydrazine, hydrazine hydrate, methylhydrazine, phenylhydrazine, amines such as methylamine or methylethylenediamine, sodium sulfide, or the like may be listed.

The amount of use of the amine or the like used in the deprotection reaction is preferably 0.5-fold or more the molar amount, and more preferably 1-fold or more the molar amount, of the compound represented by general formula (2-9). Also, the amount is preferably 20-fold or less the molar amount, and more preferably 10-fold or less the molar amount, of the compound.

The type of the solvent used in the deprotection reaction is not particularly limited as long as the solvent is inactive to the reaction. Examples thereof include water, alcohol solvents, ether solvents, and aromatic hydrocarbon solvents, and these solvents may be used individually or as solvent mixtures at any ratio. The alcohol solvent may be exemplified by methanol, ethanol, 2-propanol, or the like. The ether solvent may be exemplified by tetrahydrofuran, diethyl ether, 1,4-dioxane, or the like; and the aromatic hydrocarbon solvent may be exemplified by toluene, xylene, or the like. Preferred examples include water, methanol, ethanol, tetrahydrofuran, toluene, or solvent mixtures of these solvents at any ratio.

For the deprotection reaction, the reaction temperature is preferably 0° C. or higher. Also, the temperature is preferably the boiling point of the solvent used in the reaction, or below.

In the reaction scheme for Production Method C aforementioned, a compound represented by general formula (N2-5), which is a compound of the general formula (2-1A) wherein V in Y^{A} is an oxygen atom, can be produced, for example, by the method shown in the reaction scheme for Production Method N1:

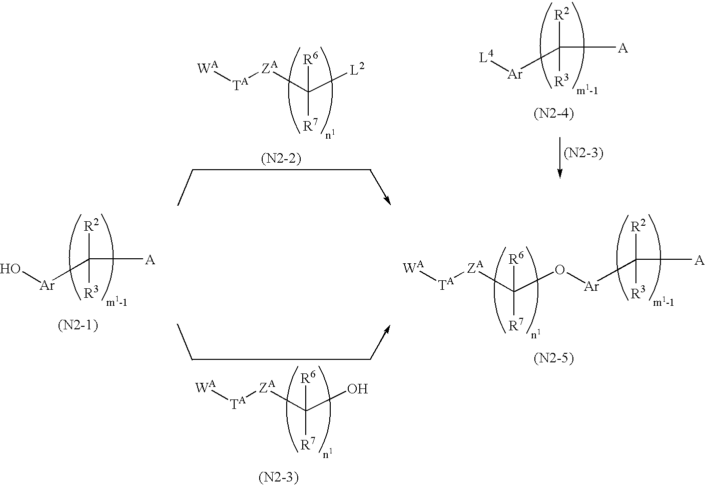

wherein R², R³, R⁶, R⁷, Ar, m¹, L² and A have the same meanings as the defined above; W^{A}, T^{A} and Z^{A} have the same meanings as the defined above for W, T and Z, respectively; n¹ is 1 or 2; and L⁴ represents a leaving group. The leaving group, L⁴, is preferably a bromine atom, an iodine atom, or the like.

A compound represented by general formula (N2-5) can be produced by an alkylation reaction between a compound represented by general formula (N2-1) and a compound represented by general formula (N2-2).

For the alkylation reaction, a method which is the same as the alkylation reaction described in the reaction scheme for the Production Method A may be listed, for example.

In another method for the above-described method, the compound represented by general formula (N2-5) can be produced by a Mitsunobu reaction between a compound represented by general formula (N2-1) and a compound represented by general formula (N2-3).

The azo compound used in the Mitsunobu reaction may be exemplified by ethyl azodicarboxylate, diisopropyl azodicarboxylate, N,N,N′,N′-tetramethylazodicarboxamide, N,N,N′,N′-tetraisopropylazodicarboxamide, or the like.

The amount of use of the azo compound used in the Mitsunobu reaction is preferably 0.5-fold or more the molar amount, and more preferably 1-fold or more the molar amount, of the compound represented by general formula (N2-1). Also, the amount is preferably 20-fold or less the molar amount, and more preferably 10-fold or less the molar amount, of the compound.

The phosphine reagent used in the Mitsunobu reaction may be exemplified by triphenylphosphine, tri-n-butylphosphine, or the like.

The amount of use of the phosphine reagent used in the Mitsunobu reaction is preferably 0.5-fold or more the molar amount, and more preferably 1-fold or more the molar amount, of the compound represented by general formula (N2-1). Also, the amount is preferably 20-fold or less the molar amount, and more preferably 10-fold or less the molar amount, of the compound.

The type of the solvent used in the Mitsunobu reaction is not particularly limited as long as the solvent is inactive to the reaction. Examples thereof include saturated hydrocarbon solvents, halogenated hydrocarbon solvents, ether solvents, and aromatic hydrocarbon solvents. These solvents may be used individually or as solvent mixtures at any ratio. The saturated hydrocarbon solvent may be exemplified by pentane, hexane, heptane, or cyclohexane; and the halogenated hydrocarbon solvent may be exemplified by dichloromethane, chloroform, or 1,2-dichloroethane. The ether solvent may be exemplified by tetrahydrofuran, diethylether, or 1,4-dioxane; and the aromatic hydrocarbon solvent may be exemplified by toluene, xylene, or the like. Preferred examples include hexane, dichloromethane, chloroform, tetrahydrofuran, diethyl ether, toluene, and solvent mixtures of these solvents at any ratio.

For the Mitsunobu reaction, the reaction temperature is preferably −50° C. or higher, and more preferably −30° C. or higher. Also, the temperature is preferably the boiling point of the solvent used in the reaction or below, and more preferably 30° C. or lower.

For the Mitsunobu reaction, the reaction time may vary depending on the raw material compound, base, solvent, reaction temperature or the like, but the time may be typically from 5 minutes to 6 hours, for example.

Furthermore, in another method, the compound represented by general formula (N2-5) can be produced by subjecting a compound represented by general formula (N2-4) and a compound represented by general formula (N2-3) to a coupling reaction in the presence of a base and a copper catalyst, without solvent or in an inactive solvent.

The solvent used in the coupling reaction is not particularly limited as long as the solvent is inactive to the present reaction Examples thereof include ether solvents, and pyridine solvents. The ether solvent may be exemplified by diethyl ether, dioxane, tetrahydrofuran, 1,2-dimethoxyethane, diethylene glycol dimethyl ether, or the like; and the pyridine solvent may be exemplified by pyridine, picoline, lutidine, collidine, or the like. However, a solvent-free condition is preferred. The copper catalyst used in the coupling reaction may be exemplified by cuprous iodide, cuprous bromide, cuprous oxide, cupric oxide, or the like, and cuprous oxide is preferred.

The alkali metal salt of the compound (N2-3) used in the coupling reaction is produced from the compound of general formula (N2-3), and an alkali metal or an alkali metal compound. The alkali metal may be exemplified by lithium, sodium, potassium, or the like; and the alkali metal compound may be exemplified by an alkali metal hydride such as lithium hydride, sodium hydride or potassium hydride. The compound is appropriately produced using sodium hydride. Also, when potassium iodide is used as an additive, the yield can be improved.

The reaction temperature may vary depending on the type of the raw material compound, catalyst or solvent, but typically, the temperature is preferably room temperature or above, and more preferably 60° C. or higher. Also, the temperature is preferably 150° C. or lower, and more preferably 120° C. or lower.

The reaction time may vary depending on the type of the raw material compound, catalyst or solvent, but typically, the time is preferably 1 hour or more, and more preferably 3 hours or more. Also, the time is preferably 7 days or less, and more preferably 72 hours or less.

In the reaction scheme for Production Method C, a compound represented by general formula (N2-9), which is a compound of the general formula (2-1A) wherein V in Y^{A} is an oxygen atom and n is 0, can be produced, for example, by the method shown in the reaction scheme for Production Method N2:

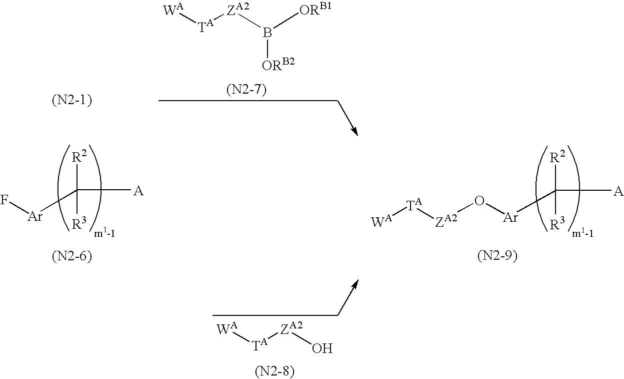

wherein R², R³, Ar, T^{A}, W^{A}, m¹ and A have the same meanings as the defined above; Z^{A2} represents a divalent group obtained by removing two hydrogen atoms from a compound selected from the group consisting of benzene, thiophene, furan, benzothiophene, isoquinoline and indazole, while these groups may be substituted with one to two X⁵s (wherein X⁵ has the same meaning as the defined above), and one or more of these groups may be protected; and R^{B1} and R^{B2}, which may be the same or different, each represent a hydrogen atom or a C1-4 alkyl group, or R^{B1} and R^{B2} may be joined to form a 1,1,2,2-tetra methylethylene group.

The compound represented by formula (N2-9) can be produced by treating the compound represented by formula (N2-1) with a compound represented by formula (N2-7) in the presence of a base and copper acetate. The base is preferably an organic base, and the organic base is preferably triethylamine, diisopropylethylamine, or the like. The solvent used in the reaction is not particularly limited as long as the solvent is inactive to the present reaction. However, the solvent is preferably a halogenated hydrocarbon solvent or the like, and the halogenated hydrocarbon solvent is preferably dichloromethane, chloroform or the like. Furthermore, if necessary, it is preferable to add molecular sieves. The reaction temperature is preferably 0° C. or higher, and also preferably the boiling point of the solvent used, or below. The reaction time may vary depending on the raw material compound, base, solvent, reaction temperature or the like, but typically, the time may be, for example, from 1 hour to 72 hours.

Furthermore, in another method, the compound represented by formula (N2-9) can also be produced by treating a compound represented by general formula (N2-6) with a compound represented by formula (N2-8) in the presence of a base. The base is preferably an inorganic base, and the inorganic base is preferably potassium carbonate, sodium carbonate or the like. The solvent used in the reaction is not particularly limited as long as the solvent is inactive to the present reaction. However, N,N-dimethylformamide or the like is preferred. The reaction temperature is preferably 0° C. or higher, and more preferably ambient temperature or more. Also, the temperature is preferably 200° C. or lower, and more preferably 160° C. or lower. The reaction time may vary depending on the raw material compound, base, solvent, reaction temperature or the like, but typically, the time may be, for example, from 1 hour to 72 hours.

In the reaction scheme for Production Method C, compounds represented by general formulas (N2-11) and (N2-16), which are compounds of the general formula (2-1A) wherein V in Y^{A} is a sulfur atom, can be produced, for example, by the method shown in the reaction scheme for Production Method N3:

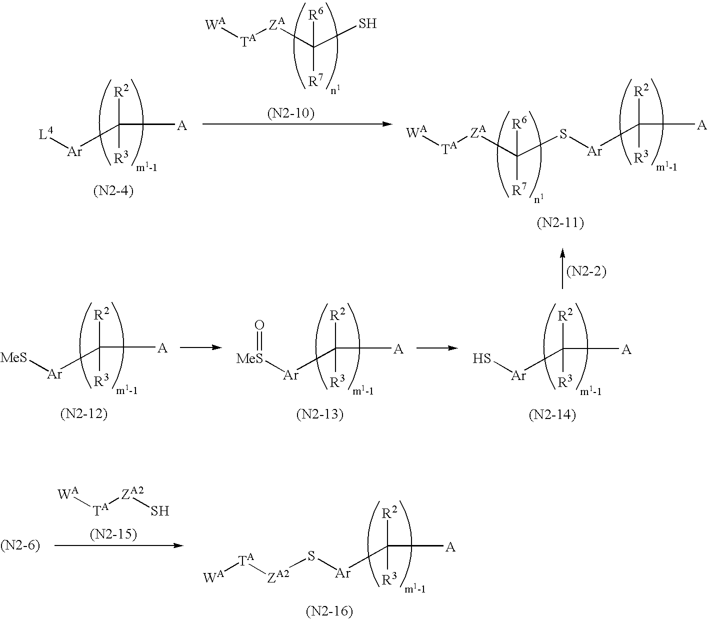

wherein R², R³, R⁵, R⁷, Ar, Z^{A}, Z^{A2}, T^{A}W^{A}, m¹, n¹, L², L², and A have the same meanings as the defined above.

A compound represented by general formula (N2-11) can be produced by subjecting a compound represented by general formula (N2-4) and a compound represented by general formula (N2-10) to a coupling reaction in the presence of a base and a copper catalyst, without solvent or in an inactive solvent. For the coupling reaction, there may be listed a method which is the same as the method for producing the compound represented by general formula (N2-5) in the reaction scheme for the Production Method N1, by subjecting the compound represented by general formula (N2-4) and the compound represented by general formula (N2-3) to a coupling reaction in the presence of a base and a copper catalyst, without solvent or in an inactive solvent.

Furthermore, in another method, the compound represented by general formula (N2-11) can also be produced from a compound of general formula (N2-12) serving as the starting raw material. A compound represented by general formula (N2-13) can be produced by an oxidation reaction of a compound represented by general formula (N2-12). The oxidizing agent used in the oxidation reaction may be exemplified by an iodine oxidizing agent, an organic peracid oxidizing agent, or the like. The iodine oxidizing agent is preferably sodium metaperiodate, and the organic peracid oxidizing agent is preferably meta-chloroperbenzoic acid or the like. For example, the solvent used for the oxidation reaction which uses sodium metaperiodate is not particularly limited as long as the solvent is inactive to the present reaction. However, water, alcohol solvents, or solvent mixtures of these solvents at any ratio are preferred, and the alcohol solvent is preferably methanol, ethanol or the like. Furthermore, solvent mixtures of these solvents and water at any ratio are also preferable. The amount of sodium metaperiodate used in the oxidation reaction is preferably 0.5-fold or more the molar amount, and more preferably 1-fold or more the molar amount, of the compound represented by general formula (N2-12). Also, the amount is preferably 20-fold or less the molar amount, and more preferably 10-fold or less the molar amount, of the compound. The reaction temperature is preferably −20° C. or higher, and preferably 50° C. or lower. The reaction time may vary depending on the raw material compound, solvent, reaction temperature or the like, but typically, the time is preferably 1 hour or more, and preferably 24 hours or less.

A compound represented by general formula (N2-14) can be produced by treating a compound represented by general formula (N2-13) with an acid anhydride such as trifluoroacetic anhydride, in an inactive solvent such as acetonitrile at preferably −20° C. or higher and 0° C. or lower, for example, in the presence of a base such as 2,6-lutidine, and decomposing the resulting Pummerer rearrangement product with an alcohol such as methanol at preferably −20° C. or higher and room temperature or lower in the presence of large excess of a base such as triethylamine.

The compound represented by general formula (N2-11) can be produced through an alkylation reaction between the compound represented by general formula (N2-14) and the compound represented by general formula (N2-2). For the alkylation reaction, a method which is the same as the alkylation reaction described in the reaction scheme for the Production Method A may be listed, for example.

Meanwhile, the compound represented by general formula (N2-16) can be produced by treating a compound represented by general formula (N2-6) with a compound represented by formula (N2-15) in the presence of a base. For the reaction, a method which is the same as the method for producing the compound represented by formula (N2-9) in the reaction scheme for the Production Method N2, by treating the compound represented by general formula (N2-6) with a compound represented by formula (N2-8) in the presence of a base.

In the reaction scheme for the Production Method C aforementioned, a compound represented by general formula (N2-20), which is a compound of the general formula (2-1A) wherein V in Y^{A} is —SCH₂— and n is 0, can be produced, for example, by the method shown in the reaction scheme for Production Method N4:

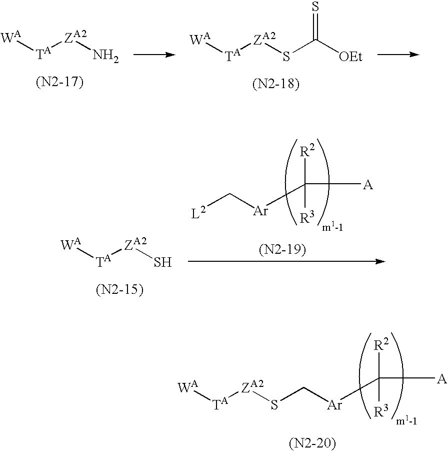

wherein R², R³, Ar, Z^{A2}, T_{A}, W^{A}, m¹, L², and A have the same meanings as the defined above.

The compound represented by general formula (N2-18) can be produced by treating a compound represented by general formula (N2-17) with sodium nitrite in water, methanol, ethanol or a solvent mixture of these solvents at any ratio, in the presence of a mineral acid such as hydrochloric acid, at from −10° C. to 5° C. to generate a diazonium salt, and then treating the diazonium salt with potassium O-ethyl dithiocarbonate at from room temperature to 80° C.

The compound represented by general formula (N2-15) can be obtained by subjecting a compound represented by general formula (N2-18) to a hydrolysis reaction in water, methanol, ethanol or a solvent mixture of these solvents at any ratio, using a base such as sodium hydroxide or potassium hydroxide, at room temperature to 100° C.

The compound represented by general formula (N2-20) can be produced through an alkylation reaction between the compound represented by general formula (N2-15) and the compound represented by general formula (N2-19). For the alkylation reaction, there may be listed, for example, a method which is the same as the alkylation reaction described in the reaction scheme for the Production Method A aforementioned.

Furthermore, for example, by oxidizing the compound represented by general formula (N2-11), (N2-16) or (N2-20) using an appropriate oxidizing agent in the method shown in the reaction scheme for Production Method N5:

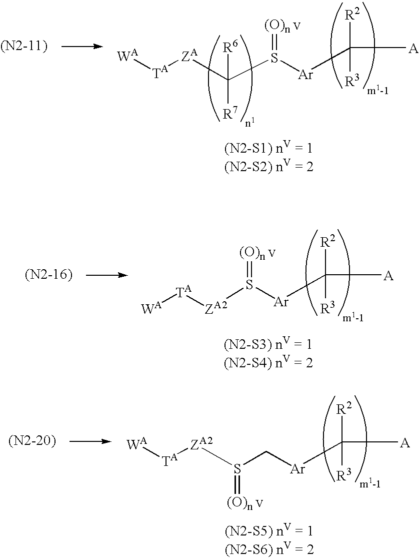

wherein R², R³, R⁶, R⁷, Ar, Z^{A}, Z^{A2}, T^{A}, W^{A}, m¹, n¹, and A have the same meanings as the defined above; and n^{V} denotes 1 or 2,

the corresponding compound represented by general formula (N2-S1), (N2-S2); (N2-S3), (N2-S4); or (N2-S5), (N2-S6) can be respectively produced.

For the reaction for producing the corresponding compound represented by general formula (N2-S1), (N2-S3) or (N2-S5) by oxidizing the compound represented by general formula (N2-11), (N2-16) or (N2-20), respectively, a method which is the same as the method for oxidizing the compound represented by general formula (N2-12) to form the compound represented by general formula (N2-13) in the reaction scheme for Production Method N3.

The oxidizing agent used in the reaction for producing the corresponding compound represented by general formula (N2-S2), (N2-S4) or (N2-S6) by oxidizing the compound represented by general formula (N2-11), (N2-16) or (N2-20), respectively, is, for example, oxone, or an organic peracid oxidizing agent. The organic peracid oxidizing agent is preferably meta-chloroperbenzoic acid, peracetic acid or the like. For example, the solvent used in the oxidation reaction using oxone is not particularly limited as long as the solvent is inactive to the present reaction. Water, alcohol solvents, or solvent mixtures of these solvents at any ratio are preferred, and the alcohol solvent is preferably methanol or ethanol. Also, a solvent mixture of these solvents and water at any ratio is also preferable. The amount of Oxone used in the oxidation reaction is preferably 1-fold or more the molar amount, and more preferably 2-fold or more the molar amount, of the compound represented by general formula (N2-11), (N2-16), or (N2-20). Also, the amount is preferably 20-fold or less the molar amount, and more preferably 10-fold or less the molar amount, of the compound. The reaction temperature is preferably −20° C. or higher, and preferably 50° C. or lower. The reaction time may vary depending on the raw material compound, solvent, reaction temperature or the like, but typically the time is preferably 1 hour or more, and preferably 3 days or less.

In the reaction scheme for Production Method C, a compound represented by general formula (N2-27), which is a compound of the general formula (2-1A) wherein V in Y^{A} is —NH— and n is 0, can be produced, for example, by the method shown in the reaction scheme for Production Method N6:

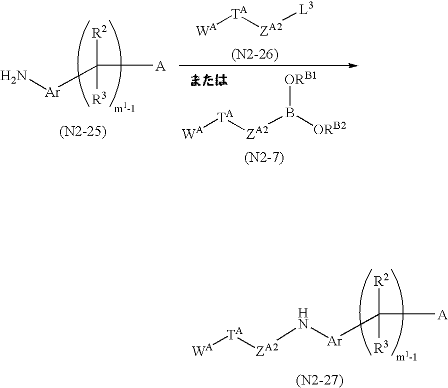

wherein R², R³, Ar, Z^{A2}, T^{A}, W^{A}, m¹, L³, R^{B1}, R^{B2}, and A have the same meanings as the defined above.

The compound represented by general formula (N2-27) can be produced by treating a compound represented by general formula (N2-25) with a compound represented by general formula (N2-26) in the presence of an acid catalyst. The acid catalyst used in the reaction is preferably pyridinium para-toluenesulfonate or the like. The solvent used in the reaction is not particularly limited as long as the solvent is inactive to the reaction. However, N,N-dimethylformamide, N,N-dimethylimidazolidinone, or the like is preferred, and it is also preferable to perform the reaction without solvent. The reaction temperature is preferably 0° C. or higher, and more preferably ambient temperature or above. Also, the temperature is preferably 200° C. or lower, and more preferably 160° C. or lower. The reaction time may vary depending on the raw material compound, solvent, reaction temperature or the like, but typically, the time may be, for example, from 1 hour to 72 hours.

Furthermore, the compound represented by general formula (N2-27) can be produced by treating the compound represented by general formula (N2-25) with the compound represented by general formula (N2-7) in the presence of a base and a copper catalyst. The copper catalyst used in the reaction is preferably anhydrous cupric acetate. The base used in the reaction is preferably an organic base, and the organic base is preferably triethylamine, diisopropylethylamine, pyridine, or the like. The solvent used in the reaction is not particularly limited as long as the solvent is inactive to the reaction. However, the solvent is preferably a halogenated hydrocarbon solvent or the like, and the halogenated hydrocarbon solvent is preferably dichloromethane, chloroform, or the like. The reaction temperature is preferably −20° C. or higher, and more preferably 0° C. or higher. Also, the temperature is preferably 50° C. or lower, and more preferably ambient temperature or below. The reaction time may vary depending on the raw material compound, solvent, reaction temperature, or the like, but typically the time may be, for example, from 1 hour to 72 hours.

In the reaction scheme for Production Method C, a compound represented by general formula (N2-30), which is a compound of the general formula (2-1A) wherein V in Y^{A} is —CONR^{V}-, and a compound represented by general formula (N2-33), which is a compound represented by general formula (2-1A) in which V is —NR^{V}CO—, can be produced, for example, by the methods shown in the reaction scheme for Production Method N7:

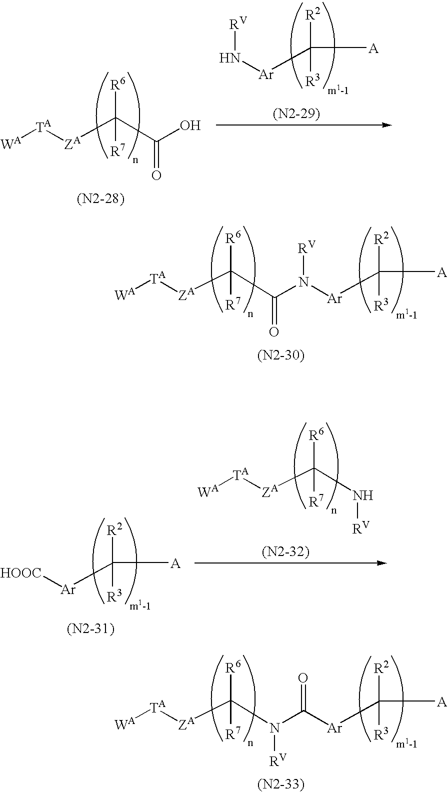

wherein R², R³, R⁶, R⁷, R^{V}, Ar, Z^{A}, T^{A}, W^{A}, m¹, n, and A have the same meanings as the defined above.

The compound represented by general formula (N2-30) can be produced by subjecting a compound represented by general formula (N2-28) and a compound represented by general formula (N2-29) to a condensation reaction in the presence of dehydrating-condensing agent. For the condensation reaction, the reaction may be performed in the co-presence of 1 to 1.5 equivalents of 1-hydroxybenzotriazole (HOBT) and/or a catalytic amount to 5 equivalents of a base, if necessary. The dehydrating-condensing agent may be exemplified by dicyclohexylcarbodiimide (DCC), 1-ethyl-3-(3-dimethylaminopropyl)carbodiimide hydrochloride (WSC), or the like. Among them, WSC is preferred. The inactive solvent used in the condensation reaction is not particularly limited as long as the solvent is inactive to the reaction. Examples thereof include nitrile solvents, amide solvents, halogenated hydrocarbon solvents, or ether solvents. These may be used as mixtures of two or more kinds of them at appropriate ratios. The nitrile solvent is preferably acetonitrile or the like; the amide solvent is preferably N,N-dimethylformamide or the like; and the ether solvent is preferably tetrahydrofuran or the like.

Examples of the base include strong bases such as hydrides of alkali metals or alkaline earth metals, amides of alkali metals or alkaline earth metals, and lower alkoxides of alkali metals or alkaline earth metals; inorganic bases such as hydroxides of alkali metals or alkaline earth metals, carbonates of alkali metals or alkaline earth metals, and hydrogen carbonates of alkali metals or alkaline earth metals; and organic bases such as organic amines or basic heterocyclic compounds. Examples of the hydrides of alkali metals or alkaline earth metals include lithium hydride, sodium hydride, potassium hydride, calcium hydride and the like; examples of the amides of alkali metals or alkaline earth metals include lithium amide, sodium amide, lithium diisopropylamide, lithium dicyclohexylamide, lithium hexamethyldisilazide, sodium hexamethyldisilazide, potassium hexamethyldisilazide and the like; and examples of the lower alkoxides of alkali metals or alkaline earth metals include sodium methoxide, sodium ethoxide, potassium tert-butoxide and the like. Examples of the hydroxides of alkali metals or alkaline earth metals include sodium hydroxide, potassium hydroxide, lithium hydroxide, barium hydroxide and the like; examples of the carbonates of alkali metals or alkaline earth metals include sodium carbonate, potassium carbonate, cesium carbonate and the like; and examples of the hydrogen carbonates of alkali metals or alkaline earth metals include sodium hydrogen carbonate, potassium hydrogen carbonate and the like. Examples of the organic amines include triethylamine, diisopropylethylamine, N-methylmorpholine, 4-dimethylaminopyridine, DBU (1,8-diazabicyclo[5.4.0]undec-7-ene), DBN (1,8-diazabicyclo[4.3.0]non-5-ene) and the like; and examples of the organic bases, such as basic heterocyclic compounds, include pyridine, imidazole, 2,6-lutidine and the like. Among the bases described above, triethylamine, diisopropylethylamine, 4-dimethylaminopyridine and the like are preferred. The reaction temperature is preferably 0° C. or higher, and preferably 30° C. or lower. The reaction time may vary depending on the raw material compound, base, solvent, reaction temperature or the like, but the time is preferably 1 hour or more, and also preferably 24 hours or less.

Furthermore, in another method for the production method described above, the compound represented by general formula (N2-30) can also be produced by allowing a reactive derivative of the compound represented by general formula (N2-28) to react with the compound represented by general formula (N2-29) in an inactive solvent. According to necessity, the reaction may be performed in the co-presence of 1 to 10 equivalents, preferably 1 to 3 equivalents, of a base. Examples of the reactive derivative of the compound represented by general formula (N2-28) include acid halides, mixed acid anhydrides, active esters and the like. The acid halide may be exemplified by acid chlorides, acid bromides, or the like; the mixed acid anhydride may be exemplified by acid an hydrides with C1-6 alkyl-carboxylic acids, C6-10 aryl-carboxylic acids, C1-6 alkylcarbonic acids or the like; and the active ester may be exemplified by esters with phenol which may be substituted, 1-hydroxybenzotriazole, N-hydroxysuccinimide or the like. The substituent of the phenol which may be substituted may be exemplified by a halogen atom (for example, fluorine, chlorine, bromine, or iodine), a nitro group, a C1-6 alkyl group which ma be halogenated, a C1-6 alkoxy group which may be halogenated, or the like. Specific examples of the phenol which may be substituted include phenol, pentachlorophenol, pentafluorophenol, p-nitrophenol and the like. The reactive derivative is preferably an acid halide.

The inactive solvent used in the reaction is not particularly limited as long as the solvent is inactive to the reaction. Examples thereof include ether solvents, halogenated hydrocarbon solvents, aromatic solvents, nitrile solvents, amide solvents, ketone solvents, sulfoxide solvents, and water. These may be used as mixtures of two or more kinds of them at appropriate ratios. Among them, acetonitrile, tetrahydrofuran, dichloromethane, chloroform and the like are preferred. For the base, there are used the same bases as those used when performing the aforementioned condensation reaction between the compound represented by general formula (N2-28) and the compound represented by general formula (N2-29) in the presence of a dehydrating-condensing agent. The base is preferably sodium hydride, potassium carbonate, sodium carbonate, sodium hydroxide, potassium hydroxide, sodium hydrogen carbonate, potassium hydrogen carbonate, triethylamine, pyridine, or the like. The reaction temperature is typically preferably −20° C. or higher, and preferably 50° C. or lower. The temperature is more preferably ambient temperature. The reaction time may vary depending on the raw material compound, base, solvent, reaction temperature, or the like, but typically, the time is preferably 5 minutes or more, and more preferably 1 hour or more. Also, the time is preferably 40 hours or less, and more preferably 18 hours or less.

A compound represented by general formula (N2-33) can be produced by subjecting a compound represented by general formula (N2-31) and a compound represented by general formula (N2-32) to a condensation reaction in the presence of a dehydrating-condensing agent. The condensation reaction is, for example, a method which is the same as the aforementioned method of performing a condensation reaction between the compound represented by general formula (N2-28) and the compound represented by general formula (N2-29) in the presence of a dehydrating-condensing agent.

Furthermore, in another production method, the compound represented by general formula (N2-33) can also be produced by allowing a reactive derivative of the compound represented by general formula (N2-31) to react with the compound represented by general formula (N2-32) in an inactive solvent. The reaction is, for example, a method which is the same as the aforementioned method of reacting a reactive derivative of the compound represented by general formula (N2-28) with the compound represented by general formula (N2-29) in an inactive solvent.

In the reaction scheme for Production Method C, a compound represented by general formula (N2-37), which is a compound of the general formula (2-1A) in which V in Y^{A} is —NH—, n is 0 and Z is a divalent group obtained by removing the two hydrogen atoms from the 2-position and 4-position of thiazole, can be produced by the method shown in the Reaction Scheme for Production Method N8:

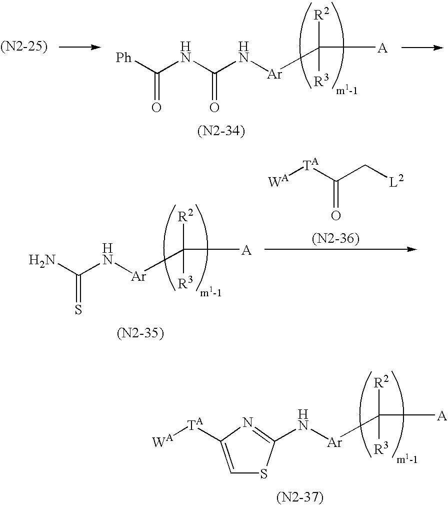

wherein R², R³, Ar, T^{A}, W^{A}, m¹, L², and A have the same meanings as the defined above.

A compound represented by general formula (N2-34) can be produced by treating the compound represented by general formula (N2-25) with benzoyl isothiocyanate. The solvent used in the reaction is not particularly limited as long as the solvent is inactive to the reaction. Examples thereof include saturated hydrocarbon solvents such as pentane, hexane, heptane, and cyclohexane; ketone solvents such as acetone; halogenated hydrocarbon solvents such as dichloromethane, chloroform, and 1,2-dichloroethane; aromatic hydrocarbon solvents such as benzene, toluene, and xylene; ether solvents such as diethyl ether, tetrahydrofuran, and 1,4-dioxane; nitrile solvents such as acetonitrile; and amide solvents such as N,N-dimethylformamide. Acetone, dichloromethane, chloroform, or toluene is preferable. The amount of benzoyl isothiocyanate used in the reaction is preferably 0.5-fold or more the molar amount, and more preferably an equimolar or more amount, of the compound represented by general formula (N2-25). Also, the amount is preferably 10-fold or less the molar amount, and more preferably 5-fold or less the molar amount, of the compound. The reaction temperature may vary depending on the raw material compound, solvent or the like, but typically, the temperature may be from 0° C. to room temperature, for example.

A compound represented by general formula (N2-35) can be produced by hydrolyzing the compound represented by general formula (N2-34). The base used in the hydrolysis reaction may be exemplified by metal hydroxides such as lithium hydroxide, sodium hydroxide, or potassium hydroxide. The solvent system used in the reaction may be exemplified by water, water-containing organic solvent systems or organic solvent systems.

The organic solvent used in the solvent system is not particularly limited as long as the solvent is inactive to the hydrolysis reaction. Examples thereof include alcohol solvents such as methanol, ethanol, 2-propanol and the like; ether solvents such as tetrahydrofuran, 1,4-dioxane and the like; or solvent mixtures of these solvents at any ratio. The amount of the base used in the reaction is preferably 0.5-fold or more the molar amount, and more preferably an equimolar or more amount, of the compound represented by general formula (N2-34). Also, the amount is preferably 50-fold or less the molar amount, and more preferably 10-fold or less the molar amount, of the compound. The reaction temperature may vary depending on the raw material compound, base, solvent or the like, but typically, for example, the temperature may be from 0° C. to the reflux temperature of the solvent.

A compound represented by general formula (N2-37) can be produced by treating the compound represented by general formula (N2-35) with the compound represented by general formula (N2-36) in an inactive solvent, optionally in the presence of a dehydrating agent. The dehydrating agent used in the reaction is, for example, anhydrous magnesium sulfate or anhydrous sodium sulfate. The solven used in the reaction is not particularly limited as long as the solvent is inactive to the reaction. The solvent is, for example, acetone or tetrahydrofuran. The reaction temperature may vary depending on the raw material compound, solvent or the like, but typically, the temperature may be from room temperature to the reflux temperature of the solvent, for example. The reaction time may vary depending on the raw material compound, solvent, reaction temperature or the like, but typically, for example, the time may be from 1 hour to 72 hours.

In the reaction scheme for Production Method C, a compound represented by general formula (O2-3), which is a compound of the general formula (2-1A) wherein V in Y^{A} is —CR^{V}═CH—, can be produced by the method shown in the reaction scheme for Production method O1:

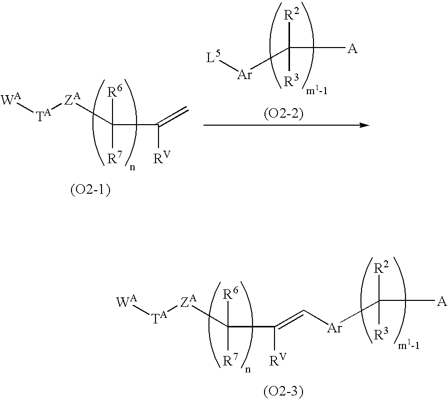

wherein R², R³, R⁶, R⁷, R^{V}, Ar, Z^{A}, T^{A}, W^{A}, m¹, n, and A have the same meanings as the defined above; and L⁵ represents a leaving group. The leaving group, L⁵, is preferably a chlorine atom, a bromine atom, an iodine atom, a trifluoromethanesulfonyloxy group, or the like.

The compound represented by general formula (O2-3) can be produced through a Heck reaction between a compound represented by general formula (O2-1) and a compound represented by general formula (O2-2). The catalyst used in the reaction may be exemplified by tetrakis(triphenylphosphine)palladium(0), palladium acetate/tri-t-butylphosphine, or the like. The base used in the reaction may be exemplified by organic amine bases or inorganic bases. The organic amine base may be exemplified by triethylamine, diisopropylethylamine or the like; and the inorganic base may be exemplified by potassium carbonate or the like. The solvent used in the reaction may be exemplified by ether solvents such as diethyl ether, tetrahydrofuran, 1,4-dioxane, or 1,2-dimethoxyethane. The reaction temperature is preferably room temperature or above, and preferably the boiling point of the solvent used, or below. The reaction time may vary depending on the raw material compound, base, solvent, reaction temperature or the like, but typically, the time is preferably 1 hour or more, and preferably 3 days or less.

In the reaction scheme for Production Method C, a compound represented by general formula (O2-5), which is a compound of the general formula (2-1A) wherein V in Y^{A} is —C≡C—, a compound represented by general formula (O2-6), which is a compound of the general formula (2-1A) wherein V is —HC═CH—, a compound represented by general formula (O2-7), which is a compound of the general formula (2-1A) wherein V is —CH₂CH₂—, a compound represented by general formula (O2-8), which is a compound of the general formula (2-1A) wherein V is —CH₂CO—, and a compound represented by general formula (O2-9), which is a compound of the general formula (2-1A) wherein V is —CH₂CH(OH)—, can be produced by the methods shown in the reaction scheme for Production Method O2:

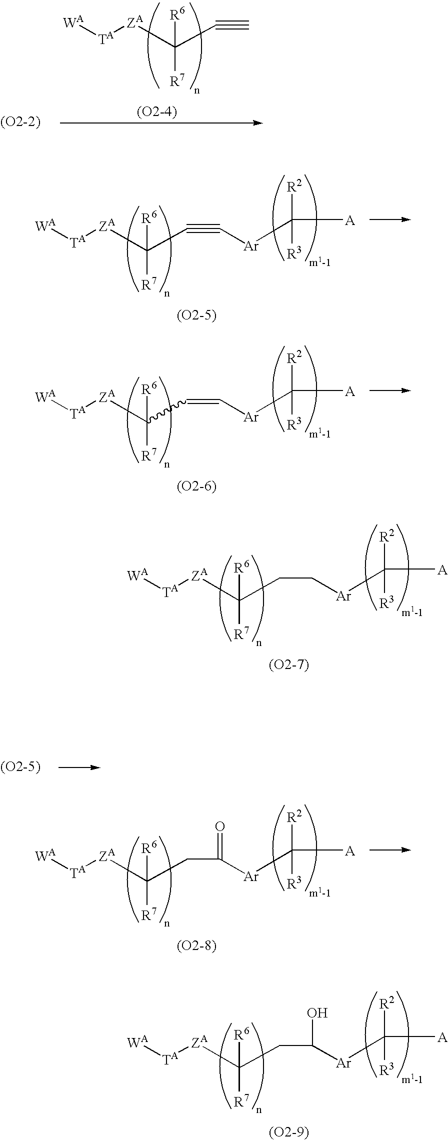

wherein R², R³, R⁶, R⁷, Ar, Z^{A}, T^{A}, W^{A}, m¹, n, L⁵ and A have the same meanings as the defined above.

The compound represented by general formula (O2-5) can be produced by subjecting the compound represented by general formula (O2-2) and the compound represented by general formula (O2-4) to a Sonogashira coupling reaction in an inactive solvent, in a nitrogen atmosphere, in the presence of a base and a palladium catalyst.

The inactive solvent used in the reaction is not particularly limited as long as the solvent is inactive to the reaction. Examples thereof include aliphatic hydrocarbon solvents such as hexane, heptane, ligroin, and petroleum ether; aromatic hydrocarbon solvents such as benzene, toluene and xylene; halogenated hydrocarbon solvents such as dichloromethane, chloroform, carbon tetrachloride, dichloroethane, chlorobenzene, and dichlorobenzene; ester solvents such as ethyl formate, ethyl acetate, propyl acetate, butyl acetate and diethyl carbonate; ether solvents such as diethyl ether, diisopropyl ether, tetrahydrofuran, dioxane, 1,2-dimethoxyethane and diethylene glycol dimethyl ether; ketone solvents such as acetone, methyl ethyl ketone, methyl isobutyl ketone, isophorone, and cyclohexanone; nitrile solvents such as acetonitrile and isobutyronitrile; amide solvents such as formamide, N,N-dimethylformamide, N,N-dimethylacetamide and hexamethyl phosphoric acid triamide; sulfoxide solvents such as dimethylsulfoxide; or sulfone solvents such as sulfolane. Ether solvents, amide solvents, or sulfoxide solvents are preferred. Moreover, ether solvents or amide solvents are most preferred. Also, by adding a small amount of water to the reaction solvent, the progress of the reaction may be accelerated.

The base used in the reaction is not particularly limited as long as it is a base conventionally used in the Sonogashira coupling reaction. Examples thereof include alkali metal carbonates such as lithium carbonate, sodium carbonate, or potassium carbonate; alkali metal bicarbonates such as lithium hydrogen carbonate, sodium hydrogen carbonate, or potassium hydrogencarbonate; alkali metal hydrides such as lithium hydride, sodium hydride, or potassium hydride; alkali metal hydroxides such as lithium hydroxide, sodium hydroxide, or potassium hydroxide; alkali metal alkoxides such as lithium methoxide, sodium methoxide, sodium ethoxide, or potassium t-butoxide; or organic amines such as triethylamine, tributylamine, diisopropylethylamine, N-methylmorpholine, pyridine, 4-(N,N-dimethylamino)pyridine, N,N-dimethylaniline, N,N-diethylaniline, 1,5-diazabicyclo[4.3.0]non-5-ene, 1,4-diazabicyclo[2.2.2]octane (DASCO), or 1,8diazabicyclo[5.4.0]-7-undecene (DBU). Among them, organic amines are preferred, and among the organic amines, triethylamine is preferred.

The palladium catalyst used in the reaction is not particularly limited as long as the catalyst is conventionally used in the Sonogashira coupling reaction. Examples thereof include palladium salts such as palladium acetate, palladium chloride, or palladium carbonate; palladium salt complexes such as dichlorobis(triphenylphosphine)-palladium complex in which palladium forms a complex with a ligand; palladium-carbon, or the like.

Also, by using cuprous iodide or benzyltriethylammonium chloride as an additive, the yield can be improved.

The reaction temperature may vary depending on the raw material compound, base, solvent or the like, but typically, the temperature may be from −20° C. to 200° C., and preferably from 0° C. to 120° C.

The reaction time may vary depending on the raw material compound, base, solvent, reaction temperature or the like, but typically, for example, the time may be from 5 minutes to 48 hours, and is preferably from 15 minutes to 24 hours.

Furthermore, a compound represented by general formula (O2-6) which is a compound of the general formula (2-1A) wherein Vis-CH═CH—, can be produced by reducing the compound represented by general formula (O2-5) (preferably, catalytic reduction in a hydrogen atmosphere).

The inactive solvent used in the catalytic reduction for the reaction is not particularly limited as long as the solvent is inactive to the present reaction. Examples thereof include aliphatic hydrocarbon solvents such as hexane, heptane, ligroin or petroleum ether; aromatic hydrocarbon solvents such as toluene, benzene, or xylene; halogenated hydrocarbon solvents such as dichloromethane, chloroform, carbon tetrachloride, dichloroethane, chlorobenzene, or dichlorobenzene; ester solvents such as methyl acetate, ethyl acetate, propyl acetate, butyl acetate, or diethyl carbonate; ether solvents such as diethyl ether, diisopropyl ether, tetrahydrofuran, dioxane, 1,2-dimethoxyethane, or diethylene glycol dimethyl ether; alcohol solvents such as methanol, ethanol, n-propanol, isopropanol, n-butanol, isobutanol, t-butanol, isoamyl alcohol, diethylene glycol, glycerin, octanol, cyclohexanol, or methyl cello solve; organic acids such as acetic acid or hydrochloric acid; water; or solvent mixtures of the above-described solvents with water. Ether solvents, or alcohol solvent are preferred, and methanol is most preferable.

The catalyst used in the catalytic reduction is not particularly limited as long as the catalyst is used conventionally in a reaction for reducing a triple bond to a double bond. However, palladium catalysts such as palladium-calcium carbonate, palladium-aluminum oxide or palladium-carbon; or rhodium catalysts such as rhodium-aluminum oxide are preferred, and palladium-calcium carbonate is more preferred.

Moreover, in the present reaction scheme, in order to reduce the ethynylene group contained in the side chain of the compound represented by general formula (O2-5) to a vinylene group, and not to reduce the ethynylene group to an ethylene group, a basic aromatic compound such as pyridine or quinoline, ammonia or an amine such as triethylamine (preferably, quinoline) may be added to the reaction solvent to inactivate the catalyst.

The hydrogen pressure is not particularly limited, but typically the reaction is performed at a pressure of 1 to 10 atmospheres, and 1 atmosphere is preferred.

The reaction temperature may vary depending on the type of the raw material compound, catalyst, solvent or the like, but typically, the temperature may be, for example, from −20° C. to 200° C., and is preferably from 0° C. to 100° C.

The reaction time may vary depending on the raw material compound, catalyst, solvent, reaction temperature or the like, but typically, the time may be, for example, from 5 minutes to 96 hours, and is preferably from 15 minutes to 72 hours.

Moreover, a compound represented by general formula (O2-7), which is a compound of the general formula (2-1A) wherein V is —CH₂CH₂—, can be produced by reducing the compound represented by general formula (O2-6) (preferably, catalytic reduction in a hydrogen atmosphere).

The inactive solvent used for the catalytic reduction in the reaction is not particularly limited as long as the solvent is inactive to the present reaction. The solvent is, for example, the same ones as those used in the above-described reduction process. Ester solvents, ether solvents, or alcohol solvents are preferred, and ethyl acetate or methanol is most preferred.

The catalyst used for the catalytic reduction in the reaction is not particularly limited as long as it is conventionally used in a catalytic reduction reaction. For example, palladium catalysts such as palladium-carbon, palladium black, palladium hydroxide, or palladium-barium sulfate; platinum catalysts such as platinum oxide, or platinum black; rhodium catalysts such as rhodium-aluminum oxide, or triphenylphosphine-rhodium chloride; or nickel catalysts such as Raney nickel may be used.

The hydrogen pressure is not particularly limited, but typically, the pressure may be from 1 to 10 atmospheres, and 1 atmosphere is preferred.

The reaction temperature may vary depending on the type of raw material compound, catalyst, solvent or the like, but typically, the temperature may be from −20° C. to 200° C., for example, and is preferably from 0° C. to 100° C.

The reaction time may vary depending on the raw material compound, catalyst, solvent, reaction temperature or the like, but typically, the time may be from 5 minutes to 96 hours, for example, and is preferably from 15 minutes to 72 hours.

Furthermore, a compound represented by general formula (O2-8), which is a compound of the general formula (2-1A) wherein V is —CH₂CO—, can be obtained by adding water to the triple bond of the compound represented by general formula (O2-5) in the presence of an acid catalyst.

The inactive solvent used in the reaction is not particularly limited as long as the solvent is inactive to the present reaction. Examples thereof include aliphatic hydrocarbon solvents such as hexane, heptane, ligroin, or petroleum ether; aromatic hydrocarbon solvents such as benzene, toluene, or xylene; halogenated hydrocarbon solvents such as dichloromethane, chloroform, carbon tetrachloride, dichloroethane, chlorobenzene, or dichlorobenzene; ester solvents such as ethyl formate, ethyl acetate, propyl acetate, butyl acetate, or diethyl carbonate; ether solvents such as diethyl ether, diisopropyl ether, tetrahydrofuran, dioxane, 1,2-dimethoxyethane, or diethylene glycol dimethyl ether; alcohol solvents such as methanol, ethanol, n-propanol, isopropanol, n-butanol, isobutanol, t-butanol, isoamyl alcohol, diethylene glycol, glycerin, octanol, cyclohexanol, or methylcellosolve; ketone solvents such as acetone, methyl ethyl ketone, methyl isobutyl ketone, isophorone, or cyclohexanone; water; or solvent mixtures of the above-listed solvents, and alcohol solvents are preferred.

The acid catalyst used in the reaction is not particularly limited as long as it is used as an acid catalyst in conventional reactions. Examples thereof include Brøonsted acids, such as inorganic acids such as hydrochloric acid, hydrobromic acid, sulfuric acid, perchloric acid, or phosphoric acid; and organic acids such as acetic acid, formic acid, oxalic acid, methanesulfonic acid, p-toluenesulfonic acid, camphor-sulfonic acid, trifluoroacetic acid, or trifluoromethanesulfonic acid; Lewis acids such as zinc chloride, tin tetrachloride, boron trichloride, boron trifluoride, or boron tribromide; or acidic ion exchange resins, and inorganic acids are preferred.

The reaction temperature may vary depending on the type of the raw material compound, catalyst, solvent or the like, but typically, the temperature may be from −20° C. to 200° C., for example, and is preferably from 0° C. to 100° C.

The reaction time may vary depending on the raw material compound, catalyst, solvent, reaction temperature or the like, but typically, the time may be from 5 minutes to 96 hours, for example, and is preferably from 15 minutes to 72 hours.

A compound represented by general formula (O2-9) can also be produced by reducing the carbonyl group of the compound represented by general formula (O2-8).

The inactive solvent used in the reaction is not particularly limited as long as the solvent is inactive to the present reaction. Examples thereof include aliphatic hydrocarbon solvents such as hexane, heptane, ligroin, or petroleum ether; aromatic hydrocarbon solvents such as benzene, toluene, or xylene; halogenated hydrocarbon solvents such as chloroform, dichloromethane, 1,2-dichloroethane, or carbon tetrachloride; ether solvents such as diethyl ether, diisopropyl ether, tetrahydrofuran, dioxane, 1,2-dimethoxyethane, or diethylene glycol dimethyl ether; alcohol solvents such as methanol, ethanol, n-propanol, isopropanol, n-butanol, isobutanol, t-butanol, isoamyl alcohol, diethylene glycol, glycerin, octanol, cyclohexanol, or methylcellosolve; or solvent mixtures of the above-listed solvents. Ether solvents or alcohol solvents are preferred, and methanol or ethanol is most preferred.

The reducing agent used in the reaction is not particularly limited as long as it is a reducing agent which can reduce a —CO— group to a —CH(OH)— group. Examples thereof include alkali metal borohydrides such as sodium borohydride, lithium borohydride, or sodium cyanoborohydride; or aluminum hydride compounds such as diisobutylaluminum hydride, lithium aluminum hydride, or lithium triethoxyaluminum hydride. Alkali metal borohydrides are preferred, and sodium borohydride is most preferred.

The reaction temperature may vary depending on the type of the raw material compound, reducing agent, solvent or the like, but typically, the temperature may be, for example, from −20° C. to 100° C., and is preferably from −10° C. to 20° C.

The reaction time may vary depending on the raw material compound, reducing agent, solvent, reaction temperature or the like, but typically, the time may be, for example, from 10 minutes to 48 hours, and is preferably from 30 minutes to 12 hours.

In the reaction scheme for Production Method C, a compound represented by general formula (O2-12) which is a compound of the general formula (2-1A) wherein V in Y^{A} is —CH(OH)—, and a compound represented by general formula (O2-13) which is a compound of the general formula (2-1A) wherein V is —CO—, can be produced by the methods shown in the reaction scheme for Production Method O3:

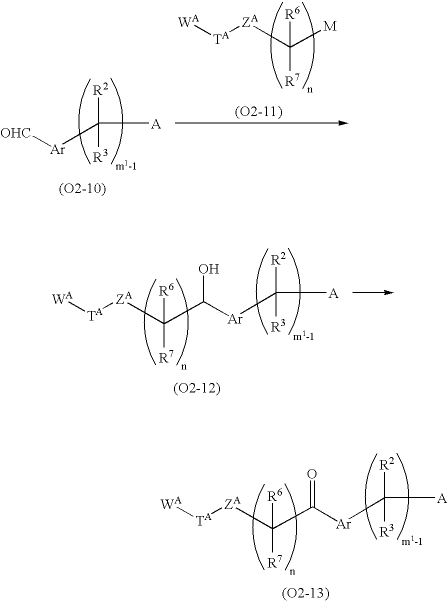

wherein R², R³, R⁶, R⁷, Ar, Z^{A}, T^{A}, W^{A}, M, m¹, n, and A have the same meanings as the defined above.

The compound represented by general formula (O2-12) can be produced through an addition reaction between the compound represented by general formula (O2-10) and the compound represented by general formula (O2-11). The solvent used in the addition reaction is not particularly limited as long as the solvent is inactive to the addition reaction. Examples thereof include aliphatic hydrocarbon solvents such as hexane, heptane, ligroin, or petroleum ether; aromatic hydrocarbon solvents such as benzene, toluene, or xylene; ether solvents such as diethyl ether, diisopropyl ether, tetrahydrofuran, 1,4-dioxane, 1,2-dimethoxyethane, or diethylene glycol dimethyl ether; or solvent mixtures of these solvents at any ratio. Hexane, toluene, diethyl ether or tetrahydrofuran is preferred.

The amount of the compound represented by general formula (O2-11) used in the addition reaction is preferably 0.5-fold or more the molar amount, and more preferably an equimolar or more amount, of the compound represented by general formula (O2-10). Also, the amount is preferably 2-fold or less the molar amount, and more preferably 1.5-fold or less the molar amount, of the compound.

The reaction temperature may vary depending on the type of the raw material compound, solvent or the like, but typically, the temperature may be from −100° C. to room temperature, for example, and is preferably from −80° C. to 0° C.

The reaction time may vary depending on the raw material compound, solvent, reaction temperature or the like, but typically, the time may be from 5 minutes to 12 hours, for example, and is preferably from 10 minutes to 6 hours.

Furthermore, the compound represented by general formula (O2-13), which is a compound of the general formula (2-1A) wherein V is —CO—, can be produced by oxidizing the compound represented by general formula (O2-12).

The oxidation reaction is, for example, a method which is the same as the method for producing the compound represented by general formula (2-1) by oxidizing the compound represented by general formula (2-1A-2) in the reaction scheme for Production Method C.

In the reaction scheme for Production Method C, a compound represented by general formula (O2-17), which is a compound of the general formula (2-1A) wherein V in Y^{A} is —CO—, and a compound represented by general formula (O2-18), which is a compound of the general formula (2-1A) wherein V is —CH₂—, can be produced by the methods shown in the reaction scheme for Production Method O4:

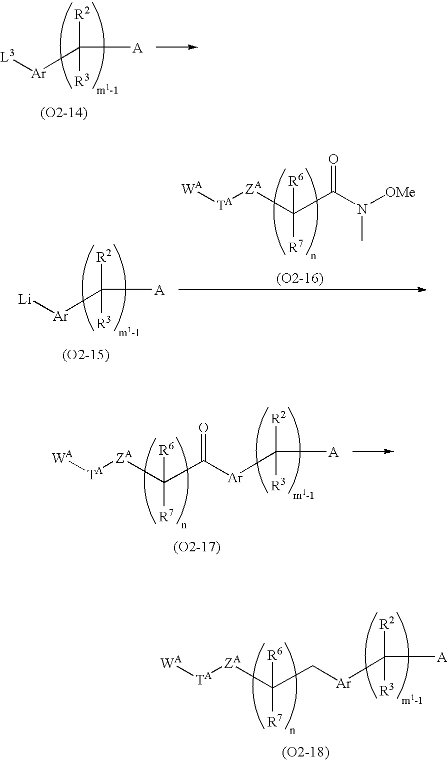

wherein R², R³, R⁶, R⁷, Ar, Z^{A}, T^{A}, W^{A}, L³, m¹, n, and A have the same meanings as the defined above.

A compound represented by general formula (O2-15) can be produced by metallizing a compound represented by general formula (O2-14) with an organometallic reagent or the like. As the group A in the compound represented by general formula (O2-14), the group represented by the general formula (2-1A-3) is preferable. A method of converting a compound represented by general formula (O2-14) wherein the group A is a group of the general formula (2-1A-1) to a compound represented by general formula (O2-14) wherein the group A is a group of the general formula (2-1A-3), can be carried out according to known acetalization methods, for example, the method described in Protective Groups in Organic Synthesis, published by John Wiley and Sons (1999). It necessary, it is preferable to add an additive such as HMPA or N,N,N′,N′-tetra methylethylenediamine to the metallization reaction. The organometallic reagent used in the metallization reaction is preferably an alkyllithium such as n-butyllithium, sec-butyllithium, or tert-butyllithium. The solvent used in the metallization reaction is not particularly limited as long as the solvent is inactive to the metallization reaction. Examples thereof include aliphatic hydrocarbon solvents such as hexane, heptane, ligroin, or petroleum ether; aromatic hydrocarbon solvents such as benzene, toluene, or xylene; ether solvents such as diethyl ether, diisopropyl ether, tetrahydrofuran, 1,4-dioxane, 1,2-dimethoxyethane, or diethylene glycol dimethyl ether; or solvent mixtures of these solvents at any ratio. Hexane, toluene, diethyl ether or tetrahydrofuran is preferred. The reaction temperature may vary depending on the type of the raw material compound, solvent or the like, but typically, the temperature may be, for example, from −100° C. to room temperature, and is preferably from −80° C. to 0° C.

The reaction time may vary depending on the raw material compound, solvent, reaction temperature or the like, but typically, the time may be, for example, from 10 minutes to 12 hours, and is preferably from 30 minutes to 6 hours.

A compound represented by general formula (O2-17) can be produced by treating the compound represented by general formula (O2-15) with a compound represented by general formula (O2-16). The solvent used in the reaction is not particularly limited as long as the solvent is inactive to the reaction. Examples thereof include aliphatic hydrocarbon solvents such as hexane, heptane, ligroin, or petroleum ether; aromatic hydrocarbon solvents such as benzene, toluene, or xylene; ether solvents such as diethyl ether, diisopropyl ether, tetrahydrofuran, 1,4-dioxane, 1,2-dimethoxyethane, or diethylene glycol dimethyl ether; or solvent mixtures of these solvents at any ratio. Hexane, toluene, diethyl ether or tetrahydrofuran is preferred. The reaction temperature may vary depending on the type of the raw material compound, solvent or the like, but typically, the temperature may be, for example, from −20° C. to 120° C., and is preferably from 0° C. to 80° C.

The reaction time may vary depending on the raw material compound, solvent, reaction temperature or the like, but typically, the time may be, for example, from 30 minutes to 24 hours, and is preferably from 1 hour to 12 hours.

A compound represented by general formula (O2-18) can be produced by reducing the compound represented by general formula (O2-17) using hydrogen gas in the presence of a metal catalyst. The metal catalyst used in the reduction reaction may be exemplified by palladium carbon, Raney nickel, platinum oxide, or the like. The solvent used in the reduction reaction is not particularly limited as long as the solvent is inactive to the reduction reaction. Examples thereof include aliphatic hydrocarbon solvents such as hexane, heptane, ligroin, or petroleum ether; aromatic hydrocarbon solvents such as toluene, benzene, or xylene; ester solvents such as methyl acetate, ethyl acetate, propyl acetate, butyl acetate or diethyl carbonate; ether solvents such as diethyl ether, diisopropyl ether, tetrahydrofuran, dioxane, 1,2-dimethoxyethane, or diethylene glycol dimethyl ether; alcohol solvents such as methanol, ethanol, n-propanol, isopropanol, n-butanol, isobutanol, t-butanol, isoamyl alcohol, diethylene glycol, glycerin, octanol, cyclohexanol, or methylcellosolve; organic acids such as acetic acid or hydrochloric acid; water; or solvent mixtures of these solvents with water at any ratio. Methanol, ethanol, ethyl acetate or hexane is preferred.

The reaction temperature may vary depending on the type of the raw material compound, catalyst, solvent or the like, but typically, the temperature may be from −0° C. to 120° C., for example, and is preferably from 0° C. to 80° C.

The reaction time may vary depending on the raw material compound, catalyst, solvent, reaction temperature or the like, but typically, the time may be from 5 minutes to 96 hours, for example, and is preferably from 15 minutes to 72 hours.

In the reaction scheme for Production Method C, a compound represented by general formula (P2-3), which is a compound of the general formula (2-1A) wherein V in Y^{A} is a phenylene group, can be produced by the method shown in the reaction scheme for Production Method P1:

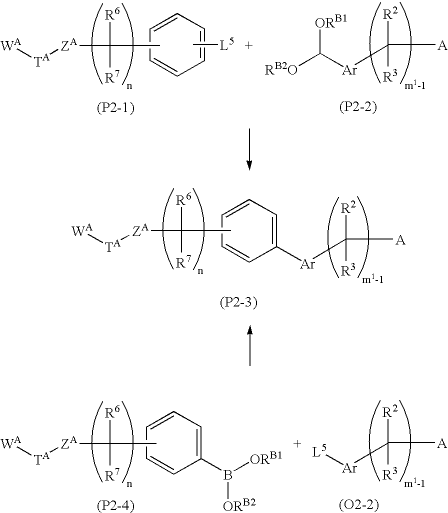

wherein R², R³, R⁶, R⁷, Ar, Z^{A}, W^{A}, m¹, n, L⁵, R^{B1}, R^{B2} and A have the same meanings as the defined above. The compound represented by general formula (P2-3) can be produced through a Suzuki reaction between a compound represented by general formula (P2-1) and a compound represented by general formula (P2-2) in the presence of a palladium catalyst. The palladium catalyst used in the Suzuki reaction may be exemplified by tetrakis(triphenylphosphine)palladium, tetrakis(methyldiphenylphosphine)palladium, dichlorobis(triphenylphosphine)palladium, dichlorobis(tri-o-tolylphosphine)palladium, dichlorobis(tricyclohexylphosphine)palladium, dichlorobis(triethylphosphine)palladium, palladium acetate, palladium chloride, bis(acetonitrile)palladium chloride, tris(dibenzylideneacetone)dipalladium, bis(diphenylphosphinoferrocene)palladium chloride, or the like. Also, a catalyst produced from palladium acetate or tris(dibenzylideneacetone) dipalladium and an arbitrary ligand, can also be used. The valence of palladium may be zero or +2. The ligand for palladium may be exemplified by phosphine ligands such as trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, or 2-(di-t-butylphosphino) biphenyl; or non-phosphine ligands such as imidazol-2-ylidenecarbenes.

The amount of the palladium catalyst used in the Suzuki reaction is preferably from 0.01 to 20% by mole, and more preferably 0.1 to 10% by mole. The base used in the Suzuki reaction may be exemplified by sodium carbonate, potassium carbonate, cesium carbonate, cesium fluoride, potassium fluoride, potassium phosphate, potassium acetate, triethylamine, potassium hydroxide, sodium hydroxide, sodium methoxide, lithium methoxide, or the like.

The solvent used in the Suzuki reaction is not particularly limited as long as the solvent is inactive to the reaction. Examples thereof include hydrocarbon solvents such as toluene, xylene or hexane; halogenated hydrocarbon solvents such as dichloromethane or chloroform; sulfoxide solvents such as dimethylsulfoxide; amide solvents such as dimethylformamide; ether solvents such as tetrahydrofuran, dioxane or diglyme; alcohol solvents such as methanol or ethanol; nitrile solvents such as acetonitrile; ketone solvents such as acetone or cyclohexanone; ester solvents such as ethyl acetate; or heterocyclic solvents such as pyridine. Also, two or more organic solvents may be used as mixtures. Furthermore, the solvent system may be any of a biphasic system of water-organic solvent, a water-containing solvent, and a homogeneous system of an organic solvent or solvents.

The reaction temperature may vary depending on the type of the raw material compound, catalyst, base, solvent or the like, but typically, the temperature may be, for example, from 0° C. to 150° C., and is preferably from room temperature to 120° C. The reaction time may vary depending on the raw material compound, catalyst, base, solvent, reaction temperature or the like, but typically, the time may be, for example, from 30 minutes to 72 hours, and is preferably from 1 hour to 48 hours.

Furthermore, in another method, the compound represented by general formula (P2-3) can be produced through a Suzuki reaction between a compound represented by general formula (P2-4) and the compound represented by general formula (O2-2) in the presence of a palladium catalyst. For the Suzuki reaction, the method as described above may be listed as an example.

In the reaction scheme for Production Method C, a compound represented by general formula (P2-7), which is a compound of the general formula (2-1A) wherein V in Y^{A} is a group obtained by removing two hydrogen atoms bound to the 3-position and 5-position of 1,2,4-oxadiazole, can be produced by the method shown in the reaction scheme for production method P2:

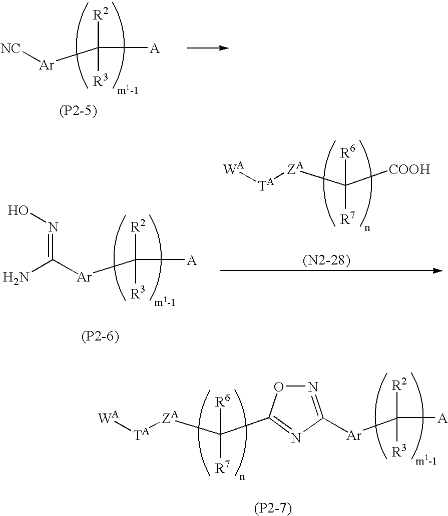

wherein R², R³, R⁶, R⁷, Ar, Z^{A}, T^{A}, W^{A}, m¹, n and A have the same meanings as the defined above.

A compound represented by general formula (P2-6) can be produced by treating a compound represented by general formula (P2-5) with hydroxylamine hydrochloride in the presence of a base.

The base used in the reaction may be exemplified by inorganic bases such as sodium hydrogen carbonate, sodium carbonate or potassium carbonate; or organic bases such as triethylamine, diisopropylethylamine or pyridine.

The organic solvent used in the reaction is not particularly limited as long as the solvent is inactive to the reaction. Examples thereof include alcohol solvents such as methanol or ethanol; ether solvents such as diethyl ether, tetrahydrofuran or 1,4-dioxane; amide solvents such as N,N-dimethylformamide; or solvent mixtures of theses solvents at any ratio.

The reaction temperature may vary depending on the raw material compound, solvent or the like, but typically, the temperature may be, for example, from room temperature to 150° C., and is preferably from room temperature to 120° C. The reaction time may vary depending on the raw material compound, solvent, reaction temperature or the like, but typically, the time may be, for example, from 30 minutes to 72 hours, and is preferably from 1 hour to 48 hours.

The compound represented by general formula (P2-7) can be produced by subjecting the compound represented by general formula (P2-6) to a condensation reaction with the compound represented by general formula (N2-28). The condensation reaction is, for example, a method which is the same as the method for producing the compound represented by general formula (N2-30) by subjecting the compound represented by general formula (N2-28) and the compound represented by general formula (N2-29) to a condensation reaction in the presence of a dehydrating-condensing agent in the reaction scheme for Production Method N7. Furthermore, in another production method, the compound represented by general formula (P2-7) can also be produced by allowing a reactive derivative of the compound represented by general formula (N2-28) to react with the compound represented by general formula (P2-6) in an inactive solvent. The reaction is, for example, a method which is the same as the method for producing the compound represented by general formula (N2-30) by allowing a reactive derivative of the compound represented by general formula (N2-28) to react with the compound represented by general formula (N2-29) in an inactive solvent.

In the reaction scheme for Production Method A, a compound represented by general formula (P2-15), which is a compound of the general formula (2-1) wherein R² is a hydrogen atom, m¹ is 1, and V in Y^{A} is a group obtained by removing two hydrogen atoms that are bound to the 2-position and 5-position of 1,3,4-thiadiazole; and a compound represented by general formula (P2-16), which is a compound of the general formula (2-1) wherein R² is a hydrogen atom, m¹ is 1, and V in Y^{A} is a group obtained by removing two hydrogen atoms that are bound to the 2-position and 5-position of 1,3,4-oxadiazole, can be produced by the methods shown in the reaction scheme for Production Method P3:

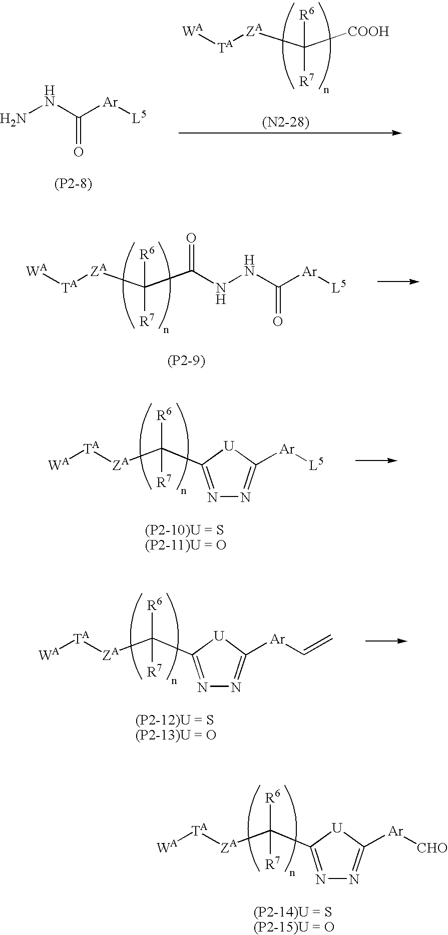

wherein R⁶, R⁷, Ar, Z^{A}, T^{A}, W^{A}, n and L⁵ have the same meanings as the defined above; and U represents a sulfur atom or an oxygen atom.

A compound represented by general formula (P2-9) can be produced by subjecting a compound represented by general formula (P2-8) and the compound represented by general formula (N2-28) to a condensation reaction in the presence of a dehydrating-condensing agent. The condensing reaction is, for example, a method which is the same as the method for producing the compound represented by general formula (N2-30) by subjecting the compound represented by general formula (N2-28) and the compound represented by general formula (N2-29) to a condensation reaction in the presence of a dehydrating-condensing agent in the reaction scheme for Production Method N7. Furthermore, in another production method, the compound represented by general formula (P2-9) can also be produced by allowing a reactive derivative of the compound represented by general formula (N2-28) to react with the compound represented by general formula (P2-8) in an inactive solvent. This reaction is, for example, a method which is the same as the method for producing the compound represented by general formula (N2-30) by allowing a reactive derivative of the compound represented by general formula (N2-28) to react with the compound represented by general formula (N2-29) in an inactive solvent which method is described in the reaction scheme for Production Method N7.

A compound represented by general formula (P2-10) can be produced by treating the compound represented by general formula (P2-9) with a sulfurizing reagent, and cyclizing the product. The sulfurizing reagent is preferably phosphorus pentasulfide, Lawson's reagent, or the like, and the Lawson's reagent is more preferred. The solvent used in the reaction is not particularly limited as long as the solvent is inactive to the reaction. Examples thereof include saturated hydrocarbon solvents such as hexane or heptane; aromatic hydrocarbon solvents such as benzene, toluene or xylene; heterocyclic solvents such as pyridine or picoline; ether solvents such as diethyl ether, tetrahydrofuran or 1,4-dioxane; nitrile solvents such as acetonitrile; or halogenated hydrocarbon solvents such as methylene chloride, chloroform or dichloroethane, and toluene, xylene, pyridine or the like is preferred. The reaction temperature may vary depending on the raw material compound, sulfurizing reagent, solvent or the like, but typically, the temperature is preferably room temperature or above, and is preferably the boiling point of the solvent used, or below. It is more preferable to perform the reaction while the reaction system is being heated to reflux the system at the boiling point of the solvent used. The reaction time may vary depending on the raw material compound, sulfurizing reagent, solvent, reaction temperature or the like, but typically, the time may be from 1 hour to 72 hours, for example.

A compound represented by general formula (P2-11) can be produced by dehydrating and cyclizing the compound represented by general formula (P2-9). The dehydrating reagent may be exemplified by combinations of a phosphine reagent and carbon tetrachloride or carbon tetrabromide, or phosphorus oxychloride, or the like. The solvent used in the cyclization reaction is not particularly limited as long as the solvent is inactive to the cyclization reaction. The solvent is, for example, a nitrile solvent such as acetonitrile. The reaction temperature may vary depending on the raw material compound, solvent or the like, but typically, the temperature may be, for example, from room temperature to the reflux temperature of the solvent, and it is preferable to perform the reaction at the reflux temperature of the solvent. The reaction time may vary depending on the raw material compound, solvent, reaction temperature or the like, but typically, the time may be from 1 hour to 72 hours, for example.

A compound represented by general formula (P2-12) or (P2-13) can be respectively produced through a Stille coupling reaction between the compound represented by general formula (P2-10) or (P2-11), and vinyltributyltin. The valence of the palladium catalyst used in the reaction may be exemplified by zero or 2, and the palladium catalyst may be exemplified by commercially available palladium catalysts, products obtained by binding a commercially available palladium catalyst with a commercially available ligand in the reaction system, or the like. Examples thereof include Pd(PPh₃)₄, BnPdCl(PPh₂), [Pd(dba)₂.PPh₃], Pd(CH₃CN)₂Cl₂, [Pd₂(dba)₃]/P^{t}Bu₃, [Pd₂(dba)₃]CHCl₃, or PdCl₂/P^{t}Bu₃. Furthermore, if necessary, metal halides such as lithium chloride, cuprous iodide or cesium fluoride may be used as an additive. The amount of the Pd catalyst used in the reaction may be, for example, 1% by mole to 20% by mole. The solvent used in the reaction is not particularly limited as long as the solvent is inactive to the reaction. Examples thereof include saturated hydrocarbon solvents such as hexane, heptane or cyclohexane; aromatic hydrocarbon solvents such as benzene, toluene or xylene; ether solvents such as diethyl ether, tetrahydrofuran, 1,4-dioxane or 1,2-dimethoxyethane; halogenated hydrocarbon solvents such as dichloromethane or chloroform; nitrile solvents such as acetonitrile; or amide solvents such as N,N-dimethylformamide. Toluene, tetrahydrofuran, 1,4-dioxane, chloroform, acetonitrile, or N,N-dimethylformamide is preferred. The reaction temperature may vary depending on the raw material compound, catalyst, solvent or the like, but typically, the temperature may be, for example, from room temperature to the reflux temperature of the solvent. The reaction time may vary depending on the raw material compound, catalyst, solvent, reaction temperature or the like, but typically, the time may be, for example, from 1 hour to 72 hours.

A compound represented by general formula (P2-14) or (P2-15) can be produced by respectively subjecting the compound represented by general formula (P2-12) or (P2-13) to Lemieux-Johnson oxidation. The oxidizing agent used in the oxidation reaction may be exemplified by a combination of osmium tetraoxide-sodium periodate, or the like. The amount of osmium tetraoxide used in the oxidation reaction may be a stoichiometric amount or a catalytic amount for the compound represented by general formula (P2-12) or (P2-13), and the catalytic amount may be, for example, 1% by mole to 20% by mole. The amount of sodium periodate used in the oxidation reaction is preferably an equimolar or more amount, and more preferably 2-fold or more the molar amount, of the compound represented by general formula (P2-12) or (P2-13). Also, the amount is preferably 10-fold or less the molar amount, and more preferably 5-fold or less the molar amount. The solvent system used in the oxidation reaction may be exemplified by water, a biphasic system of water-organic solvent, a water-containing organic solvent system, or the like. The organic solvent used in the solvent system is not particularly limited as long as the solvent is inactive to the oxidation reaction. Examples thereof include alcohol solvents such as methanol, ethanol or 2-methyl-2-propanol; ether solvents such as diethyl ether, tetrahydrofuran, or 1,4-dioxane; ketone solvents such as acetone; or solvent mixtures of these solvents at any ratio. The reaction temperature may vary depending on the raw material compound, solvent or the like, but typically, the temperature may be from 0° C. to room temperature, for example. The reaction time may vary depending on the raw material compound, solvent or the like, but typically, the time may be from 1 hour to 72 hours, for example.

In the reaction scheme for Production Method N1, a significant number of the compounds represented by general formula (N2-1) are known, and for example, can be purchased from the companies described in (Table 1) to (Table 16), or can be easily produced according to the literatures described in the footnotes of (Table 1) to (Table 16).

Furthermore, unless particularly stated otherwise, in the respective following tables including Table 1 to Table 16, the term “No.” indicates the compound number; “Suppl./Ref.” indicates the source of acquisition/literature; “HO-posit.” indicates the substitution position of a hydroxyl group; “B(OH)₂-posit.” indicates the substitution position of a —B(OH)₂ group; “NO₂-posit.” indicates the substitution position of a nitro group; and “Struct.” indicates the chemical structural formula.

TABLE 1

| | |
|---|---|
| | (N2-1-1-1) |
| 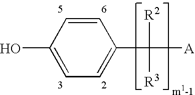 | |

| | | | | | | | Suppl./ |
|---|---|---|---|---|---|---|---|
| No. | X¹ | X¹ | m¹ | R² | R³ | A | Ref. |
| N2-1-1-1-1 | 3-MeO— | — | 1 | — | — | —CHO | TCI |
| N2-1-1-1-2 | — | — | 1 | — | — | —CHO | TCI |
| N2-1-1-1-3 | 2-OH— | — | 1 | — | — | —CHO | TCI |
| N2-1-1-1-4 | 3-OH— | — | 1 | — | — | —CHO | TCI |
| N2-1-1-1-5 | 3-EtO— | — | 1 | — | — | —CHO | TCI |
| N2-1-1-1-6 | 3-MeO— | 5-MeO— | 1 | — | — | —CHO | TCI |
| N2-1-1-1-7 | 3-tBu- | 5-tBu- | 1 | — | — | —CHO | TCI |
| N2-1-1-1-8 | 2-OH— | 3-OH— | 1 | — | — | —CHO | Wako |
| N2-1-1-1-9 | 3-Me- | — | 1 | — | — | —CHO | Aldrich |
| N2-1-1-1-10 | 3-Cl— | — | 1 | — | — | —CHO | Wako |
| N2-1-1-1-11 | 3-OH— | 5-OH— | 1 | — | — | —CHO | Wako |
| N2-1-1-1-12 | 3-Me- | 5-Me- | 1 | — | — | —CHO | TCI |
| N2-1-1-1-13 | 2-Cl— | — | 1 | — | — | —CHO | Aldrich |
| N2-1-1-1-14 | 2-MeO— | 6-MeO— | 1 | — | — | —CHO | Aldrich |
| N2-1-1-1-15 | 3-OH— | 5-MeO— | 1 | — | — | —CHO | Aldrich |
| N2-1-1-1-16 | 2-MeO— | — | 1 | — | — | —CHO | Wako |
| N2-1-1-1-17 | 2-OH— | 5-hexyl | 1 | — | — | —CHO | Salor |
| N2-1-1-1-18 | 2-OH— | 6-OH— | 1 | — | — | —CHO | Pfaltz & Bauer |
| N2-1-1-1-19 | 2-Me- | 6-Me- | 1 | — | — | —CHO | Frinton |
| N2-1-1-1-20 | 3-F— | — | 1 | — | — | —CHO | Wako |
| N2-1-1-1-21 | 3-Cl— | 5-MeO— | 1 | — | — | —CHO | Aldrich |
| N2-1-1-1-22 | 2-OH— | 5-OH— | 1 | — | — | —CHO | TCI |
| N2-1-1-1-23 | 3-CF₃— | — | 1 | — | — | —CHO | Apollo |
| N2-1-1-1-24 | 2-Me- | — | 1 | — | — | —CHO | AstaTech |
| N2-1-1-1-25 | 3-Cl— | 5-Cl— | 1 | — | — | —CHO | Maybridge |
| N2-1-1-1-26 | 3-F— | 5-MeO— | 1 | — | — | —CHO | Aldrich |
| N2-1-1-1-27 | 3-Cl— | 5-EtO— | 1 | — | — | —CHO | ART-Chem |
| N2-1-1-1-28 | 3-Cl— | 5-OH— | 1 | — | — | —CHO | Enamine |
| N2-1-1-1-29 | 2-Cl— | 5-MeO— | 1 | — | — | —CHO | ART-Chem |
| N2-1-1-1-30 | 2-Cl— | 3-OH— | 1 | — | — | —CHO | ART-Chem |
| N2-1-1-1-31 | — | — | 1 | — | — | —COMe | TCI |
| N2-1-1-1-32 | 2-OH— | — | 1 | — | — | —COMe | TCI |
| N2-1-1-1-33 | — | — | 1 | — | — | —COEt | TCI |
| N2-1-1-1-34 | 2-OH— | 5-OH— | 1 | — | — | —CO-n-Pr | Fluka |
| N2-1-1-1-35 | 2-OH— | 6-OH— | 1 | — | — | —COEt | TCI |
| N2-1-1-1-36 | 3-MeO— | — | 1 | — | — | —COMe | TCI |
| N2-1-1-1-37 | 3-MeO— | — | 1 | — | — | —COEt | Pharmeks |
| N2-1-1-1-38 | 3-MeO— | 5-MeO— | 1 | — | — | —COMe | TCI |
| N2-1-1-1-39 | 2-Me- | — | 1 | — | — | —COMe | Wako |
| N2-1-1-1-40 | 3-Me- | — | 1 | — | — | —COMe | TCI |
| N2-1-1-1-41 | 2-OH— | 3-OH— | 1 | — | — | —COMe | TCI |
| N2-1-1-1-42 | 2-OH— | 6-OH— | 1 | — | — | —COMe | TCI |
| N2-1-1-1-43 | 2-F— | — | 1 | — | — | —COMe | TCI |
| N2-1-1-1-44 | — | — | 1 | — | — | —CO-n-Bu | TCI |
| N2-1-1-1-45 | — | — | 1 | — | — | —CO-n-Pr | TCI |
| N2-1-1-1-46 | 3-tBu- | 5-tBu- | 1 | — | — | —COMe | Wako |
| N2-1-1-1-47 | 3-F— | 5-F— | 1 | — | — | —COEt | Wako |
| N2-1-1-1-48 | 2-OH— | 3-Me- | 1 | — | — | —COMe | Wako |
| N2-1-1-1-49 | 2-OH— | 3-Me- | 1 | — | — | —COEt | Wako |
| N2-1-1-1-50 | 2-OH— | — | 1 | — | — | —COEt | TCI |
| N2-1-1-1-51 | 2-OH— | 3-Me- | 1 | — | — | —COEt | Salor |
| N2-1-1-1-52 | 2-OH— | 3-Me- | 1 | — | — | —CO-n-Pr | Salor |
| N2-1-1-1-53 | 2-Me- | 5-i-Pr | 1 | — | — | —COMe | Salor |
| N2-1-1-1-54 | 2-OH— | 6-Me- | 1 | — | — | —COMe | Salor |
| N2-1-1-1-55 | 2-Me- | 5-Me- | 1 | — | — | —COMe | Salor |
| N2-1-1-1-56 | 3-OH— | — | 1 | — | — | —COMe | TCI |
| N2-1-1-1-57 | 3-OH— | — | 1 | — | — | —COEt | Wako |
| N2-1-1-1-58 | 3-OH— | 5-MeO— | 1 | — | — | —COMe | Pfaltz & Bauer |
| N2-1-1-1-59 | 3-F— | — | 1 | — | — | —COMe | Wako |
| N2-1-1-1-60 | 3-OH— | — | 1 | — | — | —CO-i-Pr | ICN |
| N2-1-1-1-61 | 3-tBu- | 5-tBu- | 1 | — | — | —COEt | Salor |
| N2-1-1-1-62 | 2-Me- | 6-Me- | 1 | — | — | —COMe | Salor |
| N2-1-1-1-63 | 2-CF₃— | — | 1 | — | — | —COMe | Apollo |
| N2-1-1-1-64 | 2-OH— | 5-n-Pr | 1 | — | — | —COMe | Maybridge |
| N2-1-1-1-65 | 2-OH— | 3-n-Pr | 1 | — | — | —COMe | Maybridge |
| N2-1-1-1-66 | 3-Cl— | — | 1 | — | — | —COEt | Maybridge |
| N2-1-1-1-67 | 2-OH— | 3-n-Pr | 1 | — | — | —COEt | Maybridge |
| N2-1-1-1-68 | 3-Me- | 5-Me- | 1 | — | — | —COMe | Aldrich |
| N2-1-1-1-69 | 3-CF₃— | — | 1 | — | — | —COMe | Apollo |
| N2-1-1-1-70 | 2-OH— | 6-OH— | 1 | — | — | —COMe | ChemPacific |
| N2-1-1-1-71 | 2-OH— | — | 1 | — | — | —CO-i-Bu | SPECS |
| N2-1-1-1-72 | 3-Cl— | 5-Me- | 1 | — | — | —COMe | TimTec |
| N2-1-1-1-73 | 3-Cl— | — | 1 | — | — | —COMe | Bionet |
| N2-1-1-1-74 | — | — | 1 | — | — | —CO-Cyclopropyl | SPECS |
| N2-1-1-1-75 | — | — | 2 | H— | H— | —CHO | (ref1) |
| N2-1-1-1-76 | 3-OH— | — | 2 | H— | H— | —CHO | (ref2) |
| N2-1-1-1-77 | 3-MeO— | — | 2 | H— | H— | —CHO | (ref3) |
| N2-1-1-1-78 | — | — | 2 | Me- | H— | —CHO | (ref4) |
| N2-1-1-1-79 | 3-MeO— | — | 2 | Et- | H— | —CHO | (ref5) |
| N2-1-1-1-80 | — | — | 2 | H— | H— | —COMe | Wako |
| N2-1-1-1-81 | 3-MeO— | — | 2 | H— | H— | —COMe | Wako |
| N2-1-1-1-82 | 3-MeO— | 5-MeO— | 2 | H— | H— | —COMe | MoleChemical |
| N2-1-1-1-83 | 3-OH— | — | 2 | H— | H— | —COMe | Synchem |
| N2-1-1-1-84 | 3-MeO— | — | 2 | H— | H— | —COOH | TCI |
| N2-1-1-1-85 | — | — | 2 | H— | H— | —COOH | TCI |
| N2-1-1-1-86 | 3-OH— | — | 2 | H— | H— | —COOH | TCI |
| N2-1-1-1-87 | — | — | 2 | H— | H— | —COOMe | TCI |
| N2-1-1-1-88 | 3-Cl— | — | 2 | H— | H— | —COOH | TCI |
| N2-1-1-1-89 | 3-F— | — | 2 | H— | H— | —COOH | Wako |
| N2-1-1-1-90 | 3-t-Bu | 5-tBu- | 2 | H— | H— | —COOH | Wako |
| N2-1-1-1-91 | — | — | 2 | Me- | H— | —COOH | TCI |
| N2-1-1-1-92 | 3-MeO— | — | 2 | H— | H— | —COOEt | Aldrich |
| N2-1-1-1-93 | — | — | 2 | H— | H— | —COOEt | Wako |
| N2-1-1-1-94 | 3-MeO— | 5-MeO— | 2 | H— | H— | —COOH | Matrix |
| N2-1-1-1-95 | 3-EtO— | — | 2 | H— | H— | —COOH | Matrix |
| N2-1-1-1-96 | — | — | 2 | i-Pr | H— | —COOH | Wako |
| N2-1-1-1-97 | 3-OH— | — | 2 | H— | H— | —COOMe | TCI |

(ref1) M. Pedras, et. al., Bioorg. Med. Chem., 11, 3115 (2003)
(ref2) J. Narayanan, et, al., Bioorg. Chem., 31, 191 (2003)
(ref3) H. Fukuda, et. al., Tetrahedron, 52, 157 (1996)
(ref4) H. Kikuchi, et. al., Chem. Lett., 1984, 341
(ref5) DE2219168

TABLE 2

| | |
|---|---|
| | (N2-1-2-1) |
| 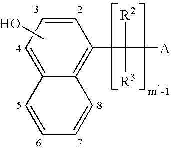 | |

| | | | | | | | | Suppl./ |
|---|---|---|---|---|---|---|---|---|
| No. | HO-posit. | X¹ | X¹ | m¹ | R² | R³ | A | Ref. |
| N2-1-2-1-1 | 4- | — | — | 1 | — | — | —CHO | Aldrich |
| N2-1-2-1-2 | 5- | — | — | 1 | — | — | —COOMe | (ref1) |
| N2-1-2-1-3 | 6- | — | — | 1 | — | — | —COOH | TCI |
| N2-1-2-1-4 | 4- | — | — | 1 | — | — | —COMe | (ref2) |
| N2-1-2-1-5 | 5- | — | — | 1 | — | — | —COMe | (ref3) |
| N2-1-2-1-6 | 4- | — | — | 2 | — | — | —COOH | (ref4) |
| N2-1-2-1-7 | 5- | — | — | 2 | — | — | —COOH | (ref5) |
| N2-1-2-1-8 | 6- | — | — | 2 | — | — | —COOH | (ref6) |

(ref1) T. Anderson, J. Am. Chem. Soc., 65, 234 (1943)
(ref2) K. Pitchumani, et al, Tetrahedron Lett., 37, 6251 (1996)
(ref3) T. Bisanz, et al., Rocz. Chem., 48, 777 (1974)
(ref4) H. Green, J. Chem. Soc., 1954, 4306
(ref5) Ogata et al., J. Org. Chem., 16, 1588 (1961)
(ref6) M. Sues, Justus Liebigs Ann. Chem., 593, 91 (1955)

TABLE 3

| | |
|---|---|
| | (N2-1-2-2) |
| 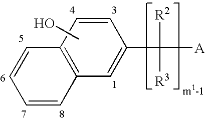 | |

| No. | HO-posit. | X¹ | X¹ | m¹ | R² | R³ | A | Suppl./Ref. |
|---|---|---|---|---|---|---|---|---|
| N2-1-2-2-1 | 4- | — | — | 1 | — | — | —CHO | Aldrich |
| N2-1-2-2-2 | 6- | — | — | 1 | — | — | —CHO | Wako |
| N2-1-2-2-3 | 6- | — | — | 1 | — | — | —COOH | TCI |
| N2-1-2-2-4 | 7- | 3-OH— | — | 1 | — | — | —COOH | TCI |
| N2-1-2-2-5 | 5- | 3-OH— | — | 1 | — | — | —COOH | TCI |
| N2-1-2-2-6 | 6- | — | — | 1 | — | — | —COOMe | TCI |
| N2-1-2-2-7 | 6- | — | — | 1 | — | — | —COMe | (ref1) |
| N2-1-2-2-8 | 6- | — | — | 2 | H— | H— | —COOMe | (ref2) |

(ref1) C. Cui, et al., J. Org. Chem., 61, 1962 (1996)
(ref2) Jones et al., J. Am. Chem. Soc., 70, 2843 (1948)

TABLE 4

| | |
|---|---|
| | (N2-1-3-1) |
| 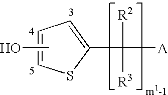 | |

| | HO- | | | | | | | Suppl./ |
|---|---|---|---|---|---|---|---|---|
| No. | posit. | X¹ | X¹ | m¹ | R² | R³ | A | Ref. |
| N2-1-3-1-1 | 4- | — | — | 1 | — | — | —COOMe | (ref1) |
| N2-1-3-1-2 | 5- | — | — | 1 | — | — | —COOMe | (ref2) |
| N2-1-3-1-3 | 4- | — | — | 2 | H— | H— | —COOEt | (ref3) |

(ref1) K. Kojima, et al., Bioorg. Med. Chem. Lett., 6, 1795 (1996)
(ref2) Jakobsen et al., Tetrahedron, 19, 1867 (1963)
(ref3) M. Chakrabarty, J. Chem. Soc., 1940, 1385

TABLE 5

| | |
|---|---|
| | (N2-1-4-2) |
| 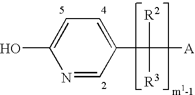 | |

| | | | | | | | Suppl./ |
|---|---|---|---|---|---|---|---|
| No. | X¹ | X¹ | m¹ | R² | R³ | A | Ref. |
| N2-1-4-2-1 | — | — | 1 | — | — | —COOH | TCI |
| N2-1-4-2-2 | 5-Cl— | — | 1 | — | — | —COOH | TCI |
| N2-1-4-2-3 | 4-OH— | — | 1 | — | — | —COOEt | SPECS |
| N2-1-4-2-4 | — | — | 1 | — | — | —COOCH₂Ph | Wako |
| N2-1-4-2-5 | — | — | 1 | — | — | —CHO | Asymchem |
| N2-1-4-2-6 | 5-Cl— | — | 1 | — | — | —CHO | Asymchem |
| N2-1-4-2-7 | 4-CF₃— | — | 1 | — | — | —COOEt | FluoroChem |
| N2-1-4-2-8 | 2-OH— | 4-Me- | 1 | — | — | —COOH | Asinex |
| N2-1-4-2-9 | 4-OH— | — | 1 | — | — | —COOH | SPECS |
| N2-1-4-2-10 | 4-OH— | 5-Cl— | 1 | — | — | —COOEt | SPECS |
| N2-1-4-2-11 | 4-CF₃— | — | 1 | — | — | —COOH | Matrix |

TABLE 6

| | |
|---|---|
| | (N2-1-5-1) |
| 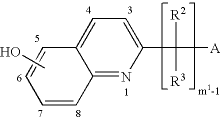 | |

| | HO- | | | | | | | Suppl./ |
|---|---|---|---|---|---|---|---|---|
| No. | posit. | X¹ | X¹ | m¹ | R² | R³ | A | Ref. |
| N2-1-5-1-1 | 5- | — | — | 1 | — | — | —CHO | (ref1) |
| N2-1-5-1-2 | 5- | — | — | 1 | — | — | —COOH | (ref2) |
| N2-1-5-1-3 | 6- | — | — | 1 | — | — | —COOMe | (ref3) |

(ref1) A. Ferranti. et. al., Farmaco, 48, 1547 (1993)
(ref2) A. Ferranti, et. al., Farmaco, 48, 1547 (1993)
(ref3) K. A. Parker, et. al., Org. Lett., 4, 4265 (2002)

TABLE 7

| | |
|---|---|
| | (N2-1-5-2) |
| 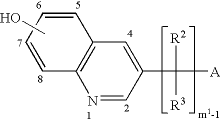 | |

| | | | | | | | | Suppl./ |
|---|---|---|---|---|---|---|---|---|
| No. | HO-posit. | X¹ | X¹ | m¹ | R² | R³ | A | Ref. |
| N2-1-5-2-1 | 6- | 4-Cl— | — | 1 | — | — | —CO-n-Pr | (ref1) |
| N2-1-5-2-2 | 6- | 2-Me- | 7-Me- | 1 | — | — | —COMe | (ref2) |
| N2-1-5-2-3 | 6- | 2-Me- | — | 1 | — | — | —COOEt | (ref3) |
| N2-1-5-2-4 | 7- | 2-Me- | — | 1 | — | — | —COOH | (ref4) |
| N2-1-5-2-5 | 7- | 2-Me- | 8-MeO— | 1 | — | — | —COMe | (ref5) |
| N2-1-5-2-6 | 7- | 4-Cl— | — | 1 | — | — | —CO-n-Pr | (ref6) |
| N2-1-5-2-7 | 7- | — | — | 1 | — | — | —CHO | (ref7) |
| N2-1-5-2-8 | 8- | 4-Cl— | — | 1 | — | — | —COEt | (ref8) |

(ref1) C. A. Leach, J. Med. Chem., 38. 2748 (1995)
(ref2) U. Kuckleander, et al., J, Prakt. Chem., 342, 17 (2000)
(ref3) E. Angeles, et al., Molecules, 6, 683 (2001)
(ref4) Yakugaku Zasshi, 79, 230 (1959)
(ref5) Ried et. al, Chem. Ber., 85, 204 (1952)
(ref6) C. A. Leach, J. Med. Chem., 38, 2748 (1995)
(ref7) l. Sato, et. al., Synthesis, 9, 1419 (2004)
(ref8) C. A. Leach, J. Med. Chem., 38, 2748 (1995)

TABLE 8

| | |
|---|---|
| | (N2-1-5-3) |
| 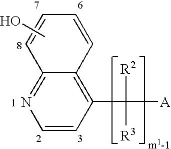 | |

| | HO- | | | | | | | Suppl./ |
|---|---|---|---|---|---|---|---|---|
| No. | posit. | X¹ | X¹ | m¹ | R² | R³ | A | Ref. |
| N2-1-5-3-1 | 7- | 2-n-Pr | — | 1 | — | — | —COOH | (ref1) |
| N2-1-5-3-2 | 8- | — | — | 1 | — | — | —CHO | (ref2) |
| N2-1-5-3-3 | 8- | — | — | 1 | — | — | —COOMe | (ref3) |
| N2-1-5-3-4 | 8- | — | — | 1 | — | — | —COOMe | (ref4) |

(ref1) Wagner-Roemmich Borsche, Justus Liebigs Ann. Chem., 544, 280 (1940)
(ref2) Mon-Yao Chen, et al., J. Chin. Chem. Soc. (Taipei), 51, 735 (2004)
(ref3) Gunatilaha et al., J. Chem. Res. Miniprint, 1979, 779
(ref4) M. Bodanszky, et al., J. Am. Chem. Soc., 86, 2478 (1964)

TABLE 9

| | | | | | | | | (N2-1-5-4) |
|---|---|---|---|---|---|---|---|---|
| 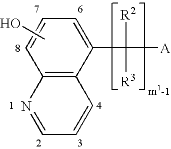 | | | | | | | | |
| | HO- | | | | | | | Suppl./ |
| No. | posit. | X¹ | X¹ | m¹ | R² | R³ | A | Ref. |
| N2-1-5-4-1 | 8- | — | — | 1 | — | — | —CHO | Oakwood |
| N2-1-5-4-2 | 8- | — | — | 1 | — | — | —COMe | Salor |
| N2-1-5-4-3 | 8- | 2-Me- | — | 1 | — | — | —CHO | (ref1) |
| N2-1-5-4-4 | 8- | — | — | 1 | — | — | —COMe | (ref2) |
| N2-1-5-4-5 | 8- | — | — | 1 | — | — | —COEt | (ref3) |
| N2-1-5-4-6 | 8- | 2-Me- | — | 1 | — | — | —COEt | (ref4) |
| N2-1-5-4-7 | 8- | 2-Me- | — | 1 | — | — | —COOH | (ref5) |

(ref1) Phillips et al., Trans. Kentucky Acad., 17, 135 (1956)
(ref2) S. Thakor, J. Indian Chem. Soc. 31, 597 (1954)
(ref3) K. Rosenmund, Arch. Pharm. (Weinheim Ger.), 279, 154 (1941)
(ref4) US2875126
(ref5) F. Zouhiri, et al., Tetrahedron Lett., 46, 2201 (2005)

TABLE 10

| | |
|---|---|
| | (N2-1-5-5) |
| 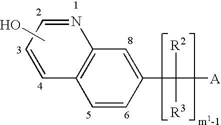 | |

| | | | | | | | | Suppl./ |
|---|---|---|---|---|---|---|---|---|
| No. | HO-posit. | X¹ | X¹ | m¹ | R² | R³ | A | Ref. |
| N2-1-5-5-1 | 4- | — | — | 1 | — | — | —COMe | (ref1) |
| N2-1-5-5-2 | 2- | — | — | 1 | — | — | —COOMe | (ref2) |

(ref1) M. S. Chodnekar et al., J. Med. Chem., 15, 49 (1972)
(ref2) C. W. Holzapfel, et al., Heterocycles, 48, 215 (1998)

TABLE 11

| | |
|---|---|
| | (N2-1-5-6) |
| 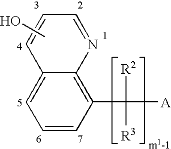 | |

| | | | | | | | | Suppl./ |
|---|---|---|---|---|---|---|---|---|
| No. | HO-posit. | X¹ | X¹ | m¹ | R² | R³ | A | Ref. |
| N2-1-5-6-1 | 5- | — | — | 1 | — | — | —COOH | (ref1) |
| N2-1-5-6-2 | 4- | — | — | 1 | — | — | —COOMe | (ref2) |

(ref1) S. Breckenridge, Can. J. Res. Sect. 8, 25, 49 (1947)
(ref2) B. Grundon, J. Am. Chem. Soc., 74, 2637 (1952)

TABLE 12

| | |
|---|---|
| | (N2-1-6-1) |
| 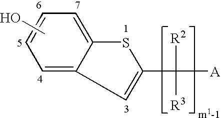 | |

| No. | HO-posit. | X¹ | X¹ | m¹ | R² | R³ | A | Suppl./Ref. |
|---|---|---|---|---|---|---|---|---|
| N2-1-6-1-1 | 4- | 3-Me- | — | 1 | — | — | —COMe | (ref1) |
| N2-1-6-1-2 | 4- | — | — | 1 | — | — | —COMe | (ref2) |
| N2-1-6-1-3 | 5- | — | — | 1 | — | — | —COOH | (ref3) |
| N2-1-6-1-4 | 5- | 3-Me- | 6-Cl— | 1 | — | — | —COOH | (ref4) |
| N2-1-6-1-5 | 5- | 6-Cl— | 7-Cl— | 1 | — | — | —CHO | (ref5) |
| N2-1-6-1-6 | 5- | 6-Cl— | 7-Cl— | 1 | — | — | —COMe | (ref6) |
| N2-1-6-1-7 | 5- | 3-Cl— | — | 1 | — | — | —COOEt | (ref7) |
| N2-1-6-1-8 | 5- | — | — | 1 | — | — | —COOMe | (ref8) |
| N2-1-6-1-9 | 5- | — | — | 1 | — | — | —COMe | (ref9) |
| N2-1-6-1-10 | 5- | — | — | 1 | — | — | —CHO | (ref10) |
| N2-1-6-1-11 | 6- | 3-Me- | — | 1 | — | — | —COOMe | (ref11) |

(ref1) Clark, et al., J. Chem. Soc. Perkin trans. 1, 1973, 1196
(ref2) P. Demerseman, et al., Bull. Soc. Chim. Fr., 1965, 1473
(ref3) Martin-Smith, et al., J. Am. Chem. Soc., 78, 5351 (1956)
(ref4) E. Campaigne, et al., J. Heterocycl. Chem., 20, 55 (1983)
(ref5) H. H. Ong, et al., J. Med. Chem., 30. 2295 (1987)
(ref6) H. H. Ong, et al., J. Med. Chem., 30, 2295 (1987)
(ref7) M. Andrew, et al., Synthesis. 7, 1181 (1999)
(ref8) J. J. Lewis, et al., J. Med. Chem., 6, 711 (1963)
(ref9) Krubsack, et al., J. Org. Chem., 40, 3179 (1975)
(ref10) J. J. Lewis, et al., J. Med. Chem., 6, 711 (1963)
(ref11) E. P. Cross, et al., J. Med. Chem., 29, 1637 (1986)

TABLE 13

| | |
|---|---|
| | (N2-1-6-2) |
| 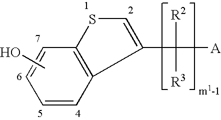 | |

| | | | | | | | | Suppl./ |
|---|---|---|---|---|---|---|---|---|
| No. | HO-posit. | X¹ | X¹ | m¹ | R² | R³ | A | Ref. |
| N2-1-6-2-1 | 5- | — | — | 1 | — | — | —CHO | (ref1) |
| N2-1-6-2-2 | 5- | — | — | 1 | — | — | —COOMe | (ref2) |
| N2-1-6-2-3 | 6- | — | — | 1 | — | — | —COOMe | (ref3) |

(ref1) Karl-Heinz. Buchheit, et al., J. Med. Chem., 38, 2331 (1995)
(ref2) S. Mitsumori, et al., J. Med. Chem., 46. 2446 (2003)
(ref3) S. Mitsumori, et al., J. Med. Chem., 46, 2446 (2003)

TABLE 14

| | | | | | | | (N2-1-6-4) | |
|---|---|---|---|---|---|---|---|---|
| 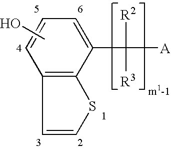 | | | | | | | | |
| No. | HO-posit. | X¹ | X¹ | m¹ | R² | R³ | A | Suppl./Ref. |
| N2-1-6-4-1 | 4- | — | — | 1 | — | — | —COMe | (ref1) |
| N2-1-6-4-2 | 4- | 5-Cl— | 6-EtO— | 1 | — | — | —CN | (ref2) |
| N2-1-6-4-3 | 4- | 3-Me- | — | 1 | — | — | —COMe | (ref3) |
| N2-1-6-4-4 | 3- | 4-Cl— | 6-Me- | 1 | — | — | —COOH | (ref4) |
| N2-1-6-4-5 | 3- | — | — | 1 | — | — | —COOH | (ref5) |

(ref1) P. D. Clark, et al., J. Chem. Soc. Perkin Trans. 1, 1982, 815
(ref2) K. Chow, et al., Tetrahedron Lett., 28, 5013 (1987)
(ref3) P. D. Clark, et al., J. Chem. Soc. Perkin Trans. 1, 1973, 1196
(ref4) DE545714
(ref5) DE539331

TABLE 15

| | |
|---|---|
| | (N2-1-7-1) |
| 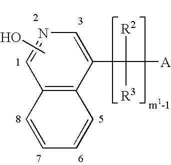 | |

| | HO- | | | | | | | Suppl./ |
|---|---|---|---|---|---|---|---|---|
| No. | posit. | X¹ | X¹ | m¹ | R² | R³ | A | Ref. |
| N2-1-7-1-1 | 1- | — | — | 1 | — | — | —COOH | (ref1) |
| N2-1-7-1-2 | 1- | — | — | 1 | — | — | —COOEt | (ref2) |
| N2-1-7-1-3 | 1- | 7- | — | 1 | — | — | —COOMe | (ref3) |
| | | MeO— | | | | | | |
| N2-1-7-1-4 | 1- | 7- | — | 1 | — | — | —COOH | (ref4) |
| | | MeO— | | | | | | |

(ref1) Dieckmann, et al., Chem. Ber., 41, 3259 (1908)
(ref2) Dieckmann, et al., Chem. Ber., 41, 3259 (1908)
(ref3) Ungnade, et al., J. Org. Chem., 10, 533 (1945)
(ref4) Ungnade, et al., J. Org. Chem., 10, 533 (1945)

TABLE 16

| | | | | | | | | (N2-1-7-2) |
|---|---|---|---|---|---|---|---|---|
| 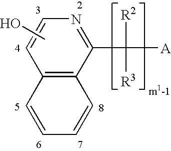 | | | | | | | | |
| | | | | | | | | Suppl./ |
| No. | HO-posit. | X¹ | X¹ | m¹ | R² | R³ | A | Ref. |
| N2-1-7-2-1 | 5- | — | — | 1 | — | — | —COOEt | (ref1) |
| N2-1-7-2-2 | 5- | — | — | 1 | — | — | —COOH | (ref2) |
| N2-1-7-2-3 | 5- | — | — | 1 | — | — | —CHO | (ref3) |

(ref1) K. Walczynski, et al., Acta Pol. Pharm., 51, 479 (1994)
(ref2) K. Walczynski, et al., Acta Pol. Pharm., 51, 479 (1994)
(ref3) Agrawal, et al., J. Med. Chem., 11, 700 (1968)

Furthermore, among the compounds represented by general formula (N2-1), the compound represented by general formula (N2-1-4-1-1) can be purchased from, for example, ABC Blocks Co., Ltd. or the like, and the compounds represented by general formula (N2-1-6-3-1), (N2-1-7-3-1), and (N2-1-8-1-1) can be easily produced according to the methods described in, for example, German Patent DE 540619, S. J. Martinez, et al., J. Chem. Soc. Perkin Transl, 1705 (1988), and Karl-Heinz Buchheit, et al., J. Med. Chem., 38, 2331 (1995), respectively.

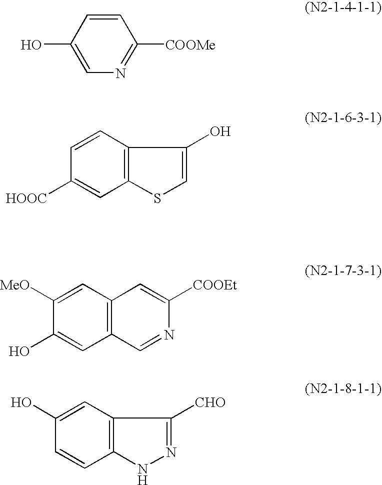

A significant number of the compounds represented by general formula (Q1), including the aforementioned general formulas (N2-4), (N2-6), (O2-2) and (O2-14):

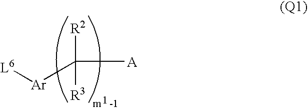

wherein Ar, A, R², R³ and m¹ have the same meanings as the defined above; L⁶ represents L³, L⁴, L⁵ or a fluorine atom; and L³, L 4 and L⁵ have the same meanings as the defined above,

are known, and among these, a compound in which L⁶ is not a trifluoromethanesulfonyloxy group can be purchased from, for example, the companies described in (Table 17) to (Table 30), or can be easily produced according to the literatures described in the footnotes of (Table 17) to (Table 30).

TABLE 17

| | |
|---|---|
| | (Q1-1-1) |
| 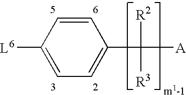 | |

| No. | L⁶ | X¹ | X¹ | m¹ | R² | R³ | A | Suppl./Ref. |
|---|---|---|---|---|---|---|---|---|
| Q1-1-1-1 | Br— | — | — | 1 | — | — | —CHO | TCI |
| Q1-1-1-2 | Cl— | — | — | 1 | — | — | —CHO | TCI |
| Q1-1-1-3 | Cl— | 2-Cl— | — | 1 | — | — | —CHO | TCI |
| Q1-1-1-4 | Cl— | 3-Cl— | — | 1 | — | — | —CHO | TCI |
| Q1-1-1-5 | F— | — | — | 1 | — | — | —CHO | TCI |
| Q1-1-1-6 | F— | 3-F— | — | 1 | — | — | —CHO | TCI |
| Q1-1-1-7 | Br— | 2-F— | — | 1 | — | — | —CHO | TCI |
| Q1-1-1-8 | Cl— | 2-F— | — | 1 | — | — | —CHO | TCI |
| Q1-1-1-9 | Cl— | 3-F— | — | 1 | — | — | —CHO | TCI |
| Q1-1-1-10 | F— | 2-Cl— | — | 1 | — | — | —CHO | TCI |
| Q1-1-1-11 | F— | 2-F— | — | 1 | — | — | —CHO | TCI |
| Q1-1-1-12 | F— | 2-MeO— | — | 1 | — | — | —CHO | Matrix |
| Q1-1-1-13 | F— | 3-Cl— | — | 1 | — | — | —CHO | TCI |
| Q1-1-1-14 | F— | 2-CF₃— | — | 1 | — | — | —CHO | Wako |
| Q1-1-1-15 | F— | 3-CF₃— | — | 1 | — | — | —CHO | Wako |
| Q1-1-1-16 | F— | 3-F— | 5-F— | 1 | — | — | —CHO | Wako |
| Q1-1-1-17 | F— | 3-F— | 5-CF₃— | 1 | — | — | —CHO | Fluorochem |
| Q1-1-1-18 | I— | — | — | 1 | — | — | —CHO | Wako |
| Q1-1-1-19 | Cl— | 2-Cl— | 6-OH— | 1 | — | — | —CHO | Matrix |
| Q1-1-1-20 | F— | 3-MeO— | — | 1 | — | — | —CHO | TCI |
| Q1-1-1-21 | Cl— | 3-CF₃— | — | 1 | — | — | —CHO | Wako |
| Q1-1-1-22 | F— | 2-F— | 5-F— | 1 | — | — | —CHO | Wako |
| Q1-1-1-23 | Cl— | 2-Cl— | 5-F— | 1 | — | — | —CHO | Lancaster |
| Q1-1-1-24 | F— | 3-Me- | — | 1 | — | — | —CHO | Wako |
| Q1-1-1-25 | F— | 2-CF₃— | 5-F— | 1 | — | — | —CHO | Apollo |
| Q1-1-1-26 | Br— | 3-F— | — | 1 | — | — | —CHO | Asymchem |
| Q1-1-1-27 | Cl— | 2-Cl— | 5-Cl— | 1 | — | — | —CHO | Chem-Impex |
| Q1-1-1-28 | I— | 3-OH— | — | 1 | — | — | —CHO | Maybridge |
| Q1-1-1-29 | F— | 2-F— | 6-OH— | 1 | — | — | —CHO | ABCR |
| Q1-1-1-30 | F— | 2-Me- | 6-Me- | 1 | — | — | —CHO | ABCR |
| Q1-1-1-31 | F— | 2-Me- | — | 1 | — | — | —CHO | ABCR |
| Q1-1-1-32 | Cl— | 2-Me- | — | 1 | — | — | —CHO | PlatteVally |
| Q1-1-1-33 | Cl— | 2-CF₃— | — | 1 | — | — | —CHO | Apollo |
| Q1-1-1-34 | Br— | 2-MeO— | 5-MeO— | 1 | — | — | —CHO | Frontier |
| Q1-1-1-35 | I— | 2-F— | — | 1 | — | — | —CHO | Ardrich |
| Q1-1-1-36 | F— | 2-OH— | — | 1 | — | — | —CHO | Apollo |
| Q1-1-1-37 | Br— | 2-F— | 6-F— | 1 | — | — | —CHO | Apollo |
| Q1-1-1-38 | Cl— | 2-F— | 6-F— | 1 | — | — | —CHO | Apollo |
| Q1-1-1-39 | Br— | 6-OH— | — | 1 | — | — | —CHO | Labotest |
| Q1-1-1-40 | Cl— | 5-Me- | — | 1 | — | — | —CHO | PlatteVally |
| Q1-1-1-41 | F— | 2-Cl— | 6-CyPrMeO— | 1 | — | — | —CHO | Matrix |
| Q1-1-1-42 | Cl— | 2-CyPrMeO— | 5-F— | 1 | — | — | —CHO | Matrix |
| Q1-1-1-43 | F— | 2-Cl— | 6-EtO— | 1 | — | — | —CHO | Matrix |
| Q1-1-1-44 | Cl— | 2-EtO— | 5-F— | 1 | — | — | —CHO | Matrix |
| Q1-1-1-45 | F— | 2-Cl— | 6-MeO— | 1 | — | — | —CHO | Matrix |
| Q1-1-1-46 | Cl— | 2-MeO— | 5-F— | 1 | — | — | —CHO | Matrix |
| Q1-1-1-47 | F— | 2-Cl— | 6-PrO— | 1 | — | — | —CHO | Matrix |
| Q1-1-1-48 | Cl— | 2-PrO— | 5-F— | 1 | — | — | —CHO | Matrix |
| Q1-1-1-49 | F— | 2-CyPrMeO— | 5-F— | 1 | — | — | —CHO | Matrix |
| Q1-1-1-50 | F— | 3-CyPrMeO— | 5-F— | 1 | — | — | —CHO | Matrix |
| Q1-1-1-51 | F— | 2-CyPrMeO— | | 1 | — | — | —CHO | Matrix |
| Q1-1-1-52 | Cl— | 2-CyPrMeO— | 5-Cl— | 1 | — | — | —CHO | Matrix |
| Q1-1-1-53 | Cl— | 2-EtO— | 5-Cl— | 1 | — | — | —CHO | Matrix |
| Q1-1-1-54 | Cl— | 2-MeO— | 5-Cl— | 1 | — | — | —CHO | Matrix |
| Q1-1-1-55 | Cl— | 2-PrO— | 5-Cl— | 1 | — | — | —CHO | Matrix |
| Q1-1-1-56 | F— | 3-F— | 5-MeO— | 1 | — | — | —CHO | Matrix |
| Q1-1-1-57 | F— | 2-MeO— | 5-F— | 1 | — | — | —CHO | Matrix |
| Q1-1-1-58 | F— | 3-F— | 5-PrO— | 1 | — | — | —CHO | Matrix |
| Q1-1-1-59 | F— | 2-PrO— | 5-F— | 1 | — | — | —CHO | Matrix |
| Q1-1-1-60 | F— | 2-EtO— | 5-F— | 1 | — | — | —CHO | Matrix |
| Q1-1-1-61 | F— | 3-EtO— | 5-F— | 1 | — | — | —CHO | Matrix |
| Q1-1-1-62 | F— | 2-EtO— | — | 1 | — | — | —CHO | Matrix |
| Q1-1-1-63 | F— | 2-PrO— | — | 1 | — | — | —CHO | Matrix |
| Q1-1-1-64 | Br— | — | — | 1 | — | — | —COMe | TCI |
| Q1-1-1-65 | Cl— | — | — | 1 | — | — | —COMe | TCI |
| Q1-1-1-66 | Cl— | 2-Cl— | 3-Cl— | 1 | — | — | —COMe | Wako |
| Q1-1-1-67 | F— | — | — | 1 | — | — | —COMe | TCI |
| Q1-1-1-68 | Cl— | 2-Cl— | — | 1 | — | — | —COMe | TCI |
| Q1-1-1-69 | Cl— | 3-Cl— | — | 1 | — | — | —COMe | TCI |
| Q1-1-1-70 | F— | 2-F— | — | 1 | — | — | —COMe | TCI |
| Q1-1-1-71 | F— | 3-F— | — | 1 | — | — | —COMe | TCI |
| Q1-1-1-72 | F— | 2-F— | 5-F— | 1 | — | — | —COMe | Wako |
| Q1-1-1-73 | I— | — | — | 1 | — | — | —COMe | TCI |
| Q1-1-1-74 | Cl— | 3-Me- | — | 1 | — | — | —COMe | Lancaster |
| Q1-1-1-75 | Cl— | 2-F— | 5-F— | 1 | — | — | —COMe | Wako |
| Q1-1-1-76 | F— | 3-Cl— | — | 1 | — | — | —COMe | Wako |
| Q1-1-1-77 | F— | 2-OH— | — | 1 | — | — | —COMe | Wako |
| Q1-1-1-78 | F— | 2-MeO— | — | 1 | — | — | —COMe | Lancaster |
| Q1-1-1-79 | F— | 2-Cl— | — | 1 | — | — | —COMe | Apollo |
| Q1-1-1-80 | F— | 2-Cl— | 5-F— | 1 | — | — | —COMe | TCI |
| Q1-1-1-81 | F— | 3-F— | 5-CF₃— | 1 | — | — | —COMe | Indofine |
| Q1-1-1-82 | F— | 2-F— | 3-F— | 1 | — | — | —COMe | Fluorochem |
| Q1-1-1-83 | F— | 3-F— | 5-F— | 1 | — | — | —COMe | Wako |
| Q1-1-1-84 | F— | 2-Cl— | 5-F— | 1 | — | — | —COMe | Matrix |
| Q1-1-1-85 | F— | 2-CF₃— | — | 1 | — | — | —COMe | Wako |
| Q1-1-1-86 | Cl— | 2-F— | 5-Me- | 1 | — | — | —COMe | Wako |
| Q1-1-1-87 | F— | 3-CF₃— | — | 1 | — | — | —COMe | Wako |
| Q1-1-1-88 | F— | 2-F— | 6-F— | 1 | — | — | —COMe | Wako |
| Q1-1-1-89 | Cl— | 2-MeO— | 5-Cl— | 1 | — | — | —COMe | Oakwood |
| Q1-1-1-90 | F— | 2-F— | 3-MeO— | 1 | — | — | —COMe | ChemPacific |
| Q1-1-1-91 | Cl— | 3-CF₃— | — | 1 | — | — | —COMe | Wako |
| Q1-1-1-92 | Br— | 2-F— | — | 1 | — | — | —COMe | Asymchem |
| Q1-1-1-93 | Br— | 3-Me- | — | 1 | — | — | —COMe | ASDI |
| Q1-1-1-94 | Cl— | 2-OH— | — | 1 | — | — | —COMe | Wako |
| Q1-1-1-95 | Br— | — | — | 1 | — | — | —COEt | TCI |
| Q1-1-1-96 | Cl— | — | — | 1 | — | — | —COEt | TCI |
| Q1-1-1-97 | Cl— | 3-Cl— | — | 1 | — | — | —COEt | Wako |
| Q1-1-1-98 | F— | — | — | 1 | — | — | —COEt | Wako |
| Q1-1-1-99 | Cl— | 2-Cl— | — | 1 | — | — | —COEt | Wako |
| Q1-1-1-100 | F— | 2-F— | — | 1 | — | — | —COEt | TCI |
| Q1-1-1-101 | Br— | 2-OH— | — | 1 | — | — | —COEt | Bionet |
| Q1-1-1-102 | F— | 3-F— | — | 1 | — | — | —COEt | Wako |
| Q1-1-1-103 | F— | 3-CF₃— | — | 1 | — | — | —COEt | Wako |
| Q1-1-1-104 | F— | 2-CF₃— | — | 1 | — | — | —COEt | Fluorochem |
| Q1-1-1-105 | F— | 2-F— | 3-F— | 1 | — | — | —COEt | Matrix |
| Q1-1-1-106 | F— | 2-F— | 5-F— | 1 | — | — | —COEt | Wako |
| Q1-1-1-107 | F— | 3-F— | 5-F— | 1 | — | — | —COEt | Wako |
| Q1-1-1-108 | F— | 2-Cl— | | 1 | — | — | —COEt | ICN |
| Q1-1-1-109 | F— | 2-F— | 6-F— | 1 | — | — | —COEt | Fluorochem |
| Q1-1-1-110 | Cl— | 2-OH— | — | 1 | — | — | —COEt | Maybridge |
| Q1-1-1-111 | F— | 3-Cl— | — | 1 | — | — | —COEt | Apollo |
| Q1-1-1-112 | F— | — | — | 1 | — | — | —COOMe | TCI |
| Q1-1-1-113 | Cl— | — | — | 1 | — | — | —COOMe | TCI |
| Q1-1-1-114 | I— | — | — | 1 | — | — | —COOMe | TCI |
| Q1-1-1-115 | Br— | — | — | 1 | — | — | —COOMe | TCI |
| Q1-1-1-116 | F— | 2-Cl— | — | 1 | — | — | —COOMe | Wako |
| Q1-1-1-117 | Br— | 3-Me- | — | 1 | — | — | —COOMe | TCI |
| Q1-1-1-118 | F— | 3-F— | — | 1 | — | — | —COOMe | Wako |
| Q1-1-1-119 | Cl— | 3-Cl— | — | 1 | — | — | —COOMe | Wako |
| Q1-1-1-120 | Cl— | 2-Cl— | — | 1 | — | — | —COOMe | TCI |
| Q1-1-1-121 | Cl— | 2-Cl— | 5-F— | 1 | — | — | —COOMe | Aldrich |
| Q1-1-1-122 | I— | 2-Me- | 6-Me- | 1 | — | — | —COOMe | Salor |
| Q1-1-1-123 | Cl— | 3-Me- | — | 1 | — | — | —COOMe | Lancaster |
| Q1-1-1-124 | F— | 2-OH— | — | 1 | — | — | —COOMe | Lancaster |
| Q1-1-1-125 | F— | 2-F— | — | 1 | — | — | —COOMe | Wako |
| Q1-1-1-126 | Cl— | 3-F— | — | 1 | — | — | —COOMe | TECH |
| Q1-1-1-127 | F— | 3-F— | 5-F— | 1 | — | — | —COOMe | Bionet |
| Q1-1-1-128 | Cl— | 2-F— | — | 1 | — | — | —COOMe | Wako |
| Q1-1-1-129 | Cl— | 2-OH— | — | 1 | — | — | —COOMe | Buttpark |
| Q1-1-1-130 | I— | 3-Me- | — | 1 | — | — | —COOMe | TCI |
| Q1-1-1-131 | I— | 3-Cl— | — | 1 | — | — | —COOEt | Buttpark |
| Q1-1-1-132 | Cl— | — | — | 1 | — | — | —CN | TCI |
| Q1-1-1-133 | F— | — | — | 1 | — | — | —CN | TCI |
| Q1-1-1-134 | F— | 3-F— | — | 1 | — | — | —CN | TCI |
| Q1-1-1-135 | Br— | 2-F— | — | 1 | — | — | —CN | Wako |
| Q1-1-1-136 | Cl— | 2-F— | — | 1 | — | — | —CN | Wako |
| Q1-1-1-137 | Cl— | 3-Cl— | — | 1 | — | — | —CN | Wako |
| Q1-1-1-138 | F— | 2-F— | 5-F— | 1 | — | — | —CN | Wako |
| Q1-1-1-139 | Cl— | 2-Cl— | 6-Me- | 1 | — | — | —CN | Lancaster |
| Q1-1-1-140 | F— | 2-F— | — | 1 | — | — | —CN | TCI |
| Q1-1-1-141 | Cl— | 2-Cl— | 6-Cl | 1 | — | — | —CN | TCI |
| Q1-1-1-142 | F— | 2-Cl— | — | 1 | — | — | —CN | Wako |
| Q1-1-1-143 | F— | 3-Cl— | — | 1 | — | — | —CN | Wako |
| Q1-1-1-144 | Cl— | 3-CF₃— | — | 1 | — | — | —CN | Wako |
| Q1-1-1-145 | F— | 3-CF₃— | — | 1 | — | — | —CN | Wako |
| Q1-1-1-146 | F— | 2-CF₃— | — | 1 | — | — | —CN | TCI |
| Q1-1-1-147 | Br— | 2-Cl— | — | 1 | — | — | —CN | Lancaster |
| Q1-1-1-148 | Br— | 3-Me- | — | 1 | — | — | —CN | Wako |
| Q1-1-1-149 | F— | 2-F— | 5-CF₃— | 1 | — | — | —CN | Indofine |
| Q1-1-1-150 | F— | 2-CF₃— | 5-F— | 1 | — | — | —CN | Indofine |
| Q1-1-1-151 | Br— | 3-CF₃— | — | 1 | — | — | —CN | JWPharmlab |
| Q1-1-1-152 | Br— | 3-EtO— | — | 1 | — | — | —CN | Asymchem |
| Q1-1-1-153 | Cl— | 3-Me- | — | 1 | — | — | —CN | Matrix |
| Q1-1-1-154 | Cl— | 2-CF₃— | — | 1 | — | — | —CN | Apollo |
| Q1-1-1-155 | F— | 2-Me- | — | 1 | — | — | —CN | Asychem |
| Q1-1-1-156 | F— | 3-Me- | — | 1 | — | — | —CN | Apollo |
| Q1-1-1-157 | I— | 2-F— | — | 1 | — | — | —CN | Apollo |
| Q1-1-1-158 | F— | 2-MeO— | — | 1 | — | — | —CN | Buttpark |
| Q1-1-1-159 | Cl— | 2-Cl— | 5-F— | 1 | — | — | —CN | Wako |
| Q1-1-1-160 | F— | — | — | 2 | H— | H— | —COMe | Wako |
| Q1-1-1-161 | Br— | — | — | 2 | H— | H— | —COMe | Wako |
| Q1-1-1-162 | Cl— | 3-Cl— | — | 2 | H— | H— | —COMe | TCI |
| Q1-1-1-163 | Cl— | 2-Cl— | — | 2 | H— | H— | —COMe | TCI |
| Q1-1-1-164 | Cl— | — | — | 2 | H— | H— | —COMe | Wako |
| Q1-1-1-165 | F— | 2-Cl— | — | 2 | H— | H— | —COMe | PlatteVally |
| Q1-1-1-166 | Cl— | — | — | 2 | H— | H— | —COOH | TCI |
| Q1-1-1-167 | F— | — | — | 2 | H— | H— | —COOH | TCI |
| Q1-1-1-168 | I— | — | — | 2 | H— | H— | —COOH | Lancaster |
| Q1-1-1-169 | Br— | — | — | 2 | H— | H— | —COOH | TCI |
| Q1-1-1-170 | Cl— | — | — | 2 | Me- | H— | —COOH | Aldrich |
| Q1-1-1-171 | Cl— | 2-Cl— | — | 2 | H— | H— | —COOH | TCI |
| Q1-1-1-172 | F— | — | — | 2 | Me- | H— | —COOH | Salor |
| Q1-1-1-173 | Cl— | — | — | 2 | Me- | Me- | —COOH | Lancaster |
| Q1-1-1-174 | Cl— | 3-Cl— | — | 2 | H— | H— | —COOH | TCI |
| Q1-1-1-175 | F— | 2-Cl— | — | 2 | H— | H— | —COOH | Wako |
| Q1-1-1-176 | F— | 3-F— | — | 2 | H— | H— | —COOH | Wako |
| Q1-1-1-177 | F— | 2-F— | 6-F— | 2 | H— | H— | —COOH | Wako |
| Q1-1-1-178 | F— | 2-F— | — | 2 | H— | H— | —COOH | Wako |
| Q1-1-1-179 | F— | 2-CF₃— | — | 2 | H— | H— | —COOH | Wako |
| Q1-1-1-180 | F— | 2-F— | 3-F— | 2 | H— | H— | —COOH | Wako |
| Q1-1-1-181 | F— | 2-F— | 5-F— | 2 | H— | H— | —COOH | Wako |
| Q1-1-1-182 | F— | 3-F— | 5-F— | 2 | H— | H— | —COOH | Wako |
| Q1-1-1-183 | F— | — | — | 2 | H— | H— | —COOMe | Acros |
| Q1-1-1-184 | Cl— | — | — | 2 | H— | H— | —COOEt | Salor |
| Q1-1-1-185 | Cl— | — | — | 2 | i-Pr | H— | —COOH | Aldrich |
| Q1-1-1-186 | Br— | — | — | 2 | H— | H— | —COOEt | Wako |
| Q1-1-1-187 | Cl— | 2-Cl— | — | 2 | H— | H— | —COOMe | Wako |
| Q1-1-1-188 | F— | 3-Cl— | — | 2 | H— | H— | —COOH | Wako |
| Q1-1-1-189 | Cl— | 2-Cl— | 5-F— | 2 | H— | H— | —COOH | Matrix |
| Q1-1-1-190 | Cl— | 3-Cl— | — | 2 | H— | H— | —COOMe | Wako |
| Q1-1-1-191 | Cl— | 3-Cl— | — | 2 | H— | H— | —COOEt | Wako |
| Q1-1-1-192 | F— | 2-Cl— | — | 2 | H— | H — | —COOMe | Acros |
| Q1-1-1-193 | Cl— | 2-F— | — | 2 | H— | H— | —COOH | Wako |
| Q1-1-1-194 | Cl— | 2-Cl— | — | 2 | H— | H— | —COOEt | FluoroChem |
| Q1-1-1-195 | Br— | — | — | 2 | H— | H— | —COOMe | Toronto |
| Q1-1-1-196 | Br— | — | — | 2 | Me- | Me- | —COOMe | Toronto |
| Q1-1-1-197 | Cl— | 2-CF₃— | — | 2 | H— | H— | —COOH | Matrix |
| Q1-1-1-198 | Cl— | 3-F— | — | 2 | H— | H— | —COOH | Matrix |
| Q1-1-1-199 | Br— | 2-EtO— | 5-EtO— | 2 | H— | H— | —COOH | ChemT&I |
| Q1-1-1-200 | Br— | 2-MeO— | 5-MeO— | 2 | H— | H— | —COOH | ChemT&I |
| Q1-1-1-201 | Cl— | 3-Me- | — | 2 | H— | H— | —COOH | ChemT&I |
| Q1-1-1-202 | Br— | 3-Me- | — | 2 | H— | H— | —COOH | ChemT&I |
| Q1-1-1-203 | Cl— | 2-Cl— | 3-Cl— | 2 | H— | H— | —COOH | ChemT&I |
| Q1-1-1-204 | F— | 3-CF₃— | — | 2 | H— | H— | —COOH | Matrix |
| Q1-1-1-205 | Br— | 2-F— | 6-F— | 2 | H— | H— | —COOH | Apollo |
| Q1-1-1-206 | Cl— | 2-F— | 6-F— | 2 | H— | H— | —COOH | Apollo |
| Q1-1-1-207 | F— | 2-Cl— | 5-F— | 2 | H— | H— | —COOH | Matrix |
| Q1-1-1-208 | Br— | — | — | 2 | Me- | Me- | —COOH | Ubichem |
| Q1-1-1-209 | Br— | — | — | 2 | Me- | Me- | —COO-t-Bu | Ubichem |

CyPrMeO—: Cyclopropylmethoxy-

TABLE 18

| | |
|---|---|
| | (Q1-2-1) |
| 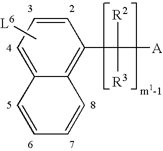 | |

| No. | L⁶ | X¹ | X¹ | m¹ | R² | R³ | A | Suppl./Ref. |
|---|---|---|---|---|---|---|---|---|
| Q1-2-1-1 | 4-F— | — | — | 1 | — | — | —CHO | Rieke |
| Q1-2-1-2 | 4-Cl— | — | — | 1 | — | — | —CHO | (ref1) |
| Q1-2-1-3 | 4-Br— | — | — | 1 | — | — | —CHO | (ref2) |
| Q1-2-1-4 | 6-Br— | 2-OH— | — | 1 | — | — | —CHO | OTAVA |
| Q1-2-1-5 | 4-F— | — | — | 1 | — | — | —COMe | Wako |
| Q1-2-1-6 | 4-Cl— | — | — | 1 | — | — | —COMe | (ref3) |
| Q1-2-1-7 | 4-Br— | — | — | 1 | — | — | —COMe | (ref4) |
| Q1-2-1-8 | 4-I— | — | — | 1 | — | — | —COMe | (ref5) |
| Q1-2-1-9 | 4-F— | — | — | 1 | — | — | —COOH | Wako |
| Q1-2-1-10 | 4-F— | — | — | 1 | — | — | —COOMe | (ref6) |
| Q1-2-1-11 | 4-Br— | — | — | 1 | — | — | —COOMe | (ref7) |
| Q1-2-1-12 | 4-I— | — | — | 1 | — | — | —COOMe | (ref8) |
| Q1-2-1-13 | 5-Br— | — | — | 1 | — | — | —COOH | TimTec |
| Q1-2-1-14 | 5-Cl— | — | — | 1 | — | — | —COOH | IBS |

(ref1) Jacobs, et al., J. Org. Chem., 11, 223 (1946)
(ref2) F. Mayer, et al., Chem. Ber., 65, 1854 (1922)
(ref3) Jacobs, et al., J. Org. Chem., 11, 27 (1946)
(ref4) J. M. Kim., et al., Can. J. Chem., 73, 885 (1995) Fujita, et al., Agric. Biol. Chem., 25,
(ref5) 719 (1961)
(ref6) W. Adcock, et al., J. Am. Chem. Soc., 89, 386 (1967)
(ref7) P. R. Bernstein, et al., Bioorg. Med. Chem. Lett., 11, 2769 (2001)
(ref8) T. Miura, et al., J. Am. Chem. Soc., 124, 518 (2002)

TABLE 19

| | |
|---|---|
| | (Q1-2-2) |
| 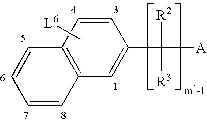 | |

| | | | | | | | | Suppl./ |
|---|---|---|---|---|---|---|---|---|
| No. | L⁶ | X¹ | X¹ | m¹ | R² | R³ | A | Ref. |
| Q1-2-2-1 | 6-Br— | — | — | 1 | — | — | —CHO | (ref1) |
| Q1-2-2-2 | 6-F— | — | — | 1 | — | — | —COMe | (ref2) |
| Q1-2-2-3 | 6-Cl— | — | — | 1 | — | — | —COMe | (ref3) |
| Q1-2-2-4 | 6-Br— | — | — | 1 | — | — | —COMe | (ref4) |
| Q1-2-2-5 | 5-Br— | 6- | — | 1 | — | — | —COMe | TCI |
| | | MeO— | | | | | | |
| Q1-2-2-6 | 6-F— | — | — | 1 | — | — | —COOMe | TCI |
| Q1-2-2-7 | 6-Cl— | — | — | 1 | — | — | —COOMe | (ref5) |
| Q1-2-2-8 | 6-Br— | — | — | 1 | — | — | —COOH | Wako |
| Q1-2-2-9 | 6-Br— | — | — | 1 | — | — | —COOMe | TCI |
| Q1-2-2-10 | 6-I— | — | — | 1 | — | — | —COOMe | (ref6) |

(ref1) S. Oida, et al., Chem. Pharm. Bull., 48, 694 (2000)
(ref2) W. Adcock, et al., J. Am. Chem. Soc., 97, 2198 (1975)
(ref3) Jacobs, et al., J. Org. Chem., 11, 27 (1946)
(ref4) Y. Tsuno, et al., Bull. Chem. Soc. Jpn., 48, 3356 (1975)
(ref5) W. Adcock, et al., Aust. J. Chem., 18, 1351 (1965)
(ref6) W. Adcock, et al., Aust. J. Chem., 18, 1351 (1965)

TABLE 20

| | |
|---|---|
| | (Q1-3-1) |
| 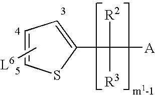 | |

| No. | L⁶ | X¹ | X¹ | m¹ | R² | R³ | A | Suppl./Ref. |
|---|---|---|---|---|---|---|---|---|
| Q1-3-1-1 | 5-Cl— | — | — | 1 | — | — | —CHO | TCI |
| Q1-3-1-2 | 5-Br— | 4-Me- | — | 1 | — | — | —CHO | Frontier |
| Q1-3-1-3 | 5-Br— | — | — | 1 | — | — | —CHO | TCI |
| Q1-3-1-4 | 5-Br— | — | — | 1 | — | — | —COMe | TCI |
| Q1-3-1-5 | 5-Cl— | — | — | 1 | — | — | —COMe | TCI |
| Q1-3-1-6 | 5-I— | — | — | 1 | — | — | —COMe | Lancaster |
| Q1-3-1-7 | 5-Cl— | — | — | 1 | — | — | —COOH | TCI |
| Q1-3-1-8 | 5-Br— | — | — | 1 | — | — | —COOH | TCI |
| Q1-3-1-9 | 5-Br— | — | — | 1 | — | — | —COOEt | Wako |
| Q1-3-1-10 | 5-Cl— | — | — | 1 | — | — | —COOEt | Lancaster |
| Q1-3-1-11 | 5-F— | — | — | 1 | — | — | —COOH | ART-Chem |
| Q1-3-1-12 | 4-Br— | — | — | 1 | — | — | —CHO | Wako |
| Q1-3-1-13 | 4-Br— | — | — | 1 | — | — | —COMe | Maybridge |
| Q1-3-1-14 | 4-Br— | — | — | 1 | — | — | —COOH | Maybridge |
| Q1-3-1-15 | 4-Br— | 5-Et- | — | 1 | — | — | —COOH | ART-Chem |
| Q1-3-1-16 | 4-Br— | 5-n-Pr- | — | 1 | — | — | —COOH | ART-Chem |
| Q1-3-1-17 | 4-Br— | 5-t-Bu- | — | 1 | — | — | —COOH | ChemT&I |
| Q1-3-1-18 | 4-Br— | 5-n-Pr- | — | 1 | — | — | —COMe | ART-Chem |
| Q1-3-1-19 | 4-Br— | 5-Me- | — | 1 | — | — | —COOH | ART-Chem |
| Q1-3-1-20 | 4-Br— | 5-Me- | — | 1 | — | — | —COEt | SPECS |
| Q1-3-1-21 | 4-Br— | 5-t-Bu- | — | 1 | — | — | —CHO | SPECS |
| Q1-3-1-22 | 4-Br— | 5-Et- | — | 1 | — | — | —CHO | ART-Chem |
| Q1-3-1-23 | 4-Br— | 5-Me- | — | 1 | — | — | —CHO | Princeton |
| Q1-3-1-24 | 5-F— | — | — | 1 | — | — | —CHO | (ref1) |
| Q1-3-1-25 | 5-I— | — | — | 1 | — | — | —CHO | (ref2) |
| Q1-3-1-26 | 4-F— | — | — | 1 | — | — | —CHO | (ref3) |
| Q1-3-1-27 | 4-Cl— | — | — | 1 | — | — | —CHO | (ref4) |

(ref1) Schuetz, et al., J. Org. Chem. 36, 2188 (1971)
(ref2) N. D'Auria, et al., J. Org. Chem., 52, 5243 (1987)
(ref3) Gronowitz, et al., Chem. Scr., 1, 33 (1971)
(ref4) O. Renault, et al,. Org. Prep. Proced. Int., 29, 488 (1997)

TABLE 21

| | | | | | | | | (Q1-3-2) |
|---|---|---|---|---|---|---|---|---|
| 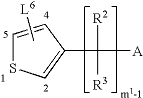 | | | | | | | | |
| | | | | | | | | Suppl./ |
| No. | L⁶ | X¹ | X¹ | m¹ | R² | R³ | A | Ref. |
| Q1-3-2-1 | 5-Cl— | — | — | 1 | — | — | —COMe | Pfaltz & |
| | | | | | | | | Bauer |
| Q1-3-2-2 | 4- | 5-Cl— | — | 1 | — | — | —COOH | Maybridge |
| | MeO— | | | | | | | |
| Q1-3-2-3 | 5-F— | — | — | 1 | — | — | —CHO | (ref1) |
| Q1-3-2-4 | 5-Br— | — | — | 1 | — | — | —CHO | (ref2) |
| Q1-3-2-5 | 5-I— | — | — | 1 | — | — | —CHO | (ref3) |

(ref1) Gronowitz, et al., Chem. Scr., 1, 33 (1971)
(ref2) N. Amishiro, et al., Chem. Pharm. Bull., 47, 1393 (1999)
(ref3) W. R. Ewing, et al., J. Med. Chem., 42, 3557 (1999)

TABLE 22

| | |
|---|---|
| | (Q1-4-1) |
| 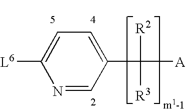 | |

| No. | L⁶ | X¹ | X¹ | m¹ | R² | R³ | A | Suppl./Ref. |
|---|---|---|---|---|---|---|---|---|
| Q1-4-1-1 | Cl— | — | — | 1 | — | — | —COOMe | Wako |
| Q1-4-1-2 | Cl— | 4-CF₃— | — | 1 | — | — | —COOMe | Matrix |
| Q1-4-1-3 | Cl— | 4-Cl— | — | 1 | — | — | —COOEt | SPECS |
| Q1-4-1-4 | Cl— | 5-Cl— | — | 1 | — | — | —COOEt | TCI |
| Q1-4-1-5 | Cl— | — | — | 1 | — | — | —CHO | Asymchem |
| Q1-4-1-6 | F— | — | — | 1 | — | — | —COOMe | Asymchem |
| Q1-4-1-7 | Cl— | 4-Cl— | — | 1 | — | — | —COMe | ChemPacific |
| Q1-4-1-8 | Cl— | — | — | 1 | — | — | —COMe | Maybridge |
| Q1-4-1-9 | Br— | — | — | 1 | — | — | —CHO | Asymchem |
| Q1-4-1-10 | Br— | — | — | 1 | — | — | —COOMe | Asymchem |
| Q1-4-1-11 | Cl— | 2-CF₃— | — | 1 | — | — | —COOH | FluoroChem |
| Q1-4-1-12 | Cl— | 4-EtO— | — | 1 | — | — | —COOH | SPECS |
| Q1-4-1-13 | Br— | 5-Cl— | — | 1 | — | — | —COOH | SPECS |
| Q1-4-1-14 | I— | — | — | 1 | — | — | —COOH | SPECS |

TABLE 23

| | |
|---|---|
| | (Q1-4-2) |
| 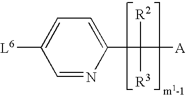 | |

| No. | L⁶ | X¹ | X¹ | m¹ | R² | R³ | A | Suppl./Ref. |
|---|---|---|---|---|---|---|---|---|
| Q1-4-2-1 | Br— | — | — | 1 | — | — | —CHO | Frontier |
| Q1-4-2-2 | Br— | — | — | 1 | — | — | —COOMe | Asymchem |
| Q1-4-2-3 | Cl— | — | — | 1 | — | — | —COOH | Matrix |
| Q1-4-2-4 | F— | — | — | 1 | — | — | —COOH | SynChem |
| Q1-4-2-5 | F— | — | — | 1 | — | — | —CHO | (ref1) |
| Q1-4-2-6 | Cl— | — | — | 1 | — | — | —CHO | (ref1) |
| Q1-4-2-7 | I— | — | — | 1 | — | — | —CHO | (ref1) |

(ref1) Blanz, et al., J. Med. Chem., 13, 1124 (1970)

TABLE 24

| | | | | | | | | (Q1-5-1) |
|---|---|---|---|---|---|---|---|---|
| 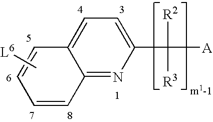 | | | | | | | | |
| | | | | | | | | Suppl./ |
| No. | L⁶ | X¹ | X¹ | m¹ | R² | R³ | A | Ref. |
| Q1-5-1-1 | 7-Cl— | 4-OH— | — | 1 | — | — | —COOH | Wako |
| Q1-5-1-2 | 5-Cl— | 8- | — | 1 | — | — | —CHO | IBS |
| | | MeO— | | | | | | |
| Q1-5-1-3 | 7-Cl— | — | — | 1 | — | — | —CHO | IBS |
| Q1-5-1-4 | 6-F— | — | — | 1 | — | — | —CHO | Asinex |
| Q1-5-1-5 | 6-Cl— | — | — | 1 | — | — | —CHO | (ref1) |
| Q1-5-1-6 | 6-Br— | — | — | 1 | — | — | —CHO | (ref2) |

(ref1) Tadros, et al., Indian J. Chem., 13, 1366 (1975)
(ref2) Mathes, et al., Chem., Ber., 90, 758 (1957)

TABLE 25

| | | | | | | | | (Q1-5-2) |
|---|---|---|---|---|---|---|---|---|
| 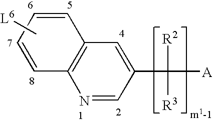 | | | | | | | | |
| | | | | | | | | Suppl./ |
| No. | L⁶ | X¹ | X¹ | m¹ | R² | R³ | A | Ref. |
| Q1-5-2-1 | 6-F— | 2-Me- | — | 1 | — | — | —COOEt | Life- |
| | | | | | | | | Chemicals |
| Q1-5-2-2 | 6-F— | 2-Me- | — | 1 | — | — | —COOH | Life- |
| | | | | | | | | Chemicals |
| Q1-5-2-3 | 7-F— | 2-Me- | — | 1 | — | — | —COOH | Enamine |
| Q1-5-2-4 | 7-Cl— | 2-Me- | — | 1 | — | — | —COOH | Enamine |
| Q1-5-2-5 | 6-Cl— | 2-Me- | — | 1 | — | — | —COOH | Enamine |
| Q1-5-2-6 | 7-Cl— | 2-Me- | — | 1 | — | — | —COOEt | BioBlocks |
| Q1-5-2-7 | 7-Br— | — | — | 1 | — | — | —CHO | (ref1) |
| Q1-5-2-8 | 7-F— | — | — | 1 | — | — | —CHO | (ref1) |

(ref1) I., Sato, et al., Synthesis, 9, 1419 (2004)

TABLE 26

| | |
|---|---|
| | (Q1-5-3) |
| 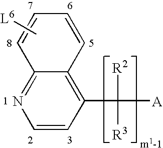 | |

| No. | L⁶ | X¹ | X¹ | m¹ | R² | R³ | A | Suppl./Ref. |
|---|---|---|---|---|---|---|---|---|
| Q1-5-3-1 | 7-Br— | 2-Me- | — | 1 | — | — | —COOEt | Salor |
| Q1-5-3-2 | 7-Cl— | 2-Me- | 8-Me- | 1 | — | — | —COOH | ChemT&I |
| Q1-5-3-3 | 7-Cl— | 2-Et- | 8-Me- | 1 | — | — | —COOH | ChemT&I |
| Q1-5-3-4 | 7-Cl— | 2-CyPr— | 8-Me- | 1 | — | — | —COOH | ChemT&I |
| Q1-5-3-5 | 8-Br— | 2-Me- | — | 1 | — | — | —COOH | ChemT&I |
| Q1-5-3-6 | 8-Br— | 2-Et- | — | 1 | — | — | —COOH | ChemT&I |
| Q1-5-3-7 | 8-Cl— | 2-Me- | — | 1 | — | — | —COOH | ChemT&I |
| Q1-5-3-8 | 8-Cl— | 2-Et- | — | 1 | — | — | —COOH | ChemT&I |
| Q1-5-3-9 | 8-F— | 2-Me- | — | 1 | — | — | —COOH | ChemT&I |
| Q1-5-3-10 | 8-F— | 2-Et- | — | 1 | — | — | —COOH | ChemT&I |
| Q1-5-3-11 | 8-Cl— | — | — | 1 | — | — | —COOEt | (ref1) |
| Q1-5-3-12 | 8-Br— | — | — | 1 | — | — | —COOH | (ref2) |
| Q1-5-3-13 | 8-Cl— | — | — | 1 | — | — | —COMe | (ref3) |

CyPr-: Cyclopropyl-
(ref1) Seibert, et al., J. Am. Chem. Soc., 68, 2121 (1946)
(ref2) Lellmann, et al., Justus Liebigs Ann. Chem., 237, 323 (1887)
(ref3) Popoff, et al., J. Med. Chem., 13, 1002 (1970)

TABLE 27

| | | | | | | | | (Q1-6-1) |
|---|---|---|---|---|---|---|---|---|
| 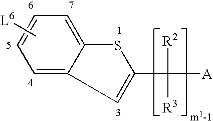 | | | | | | | | |
| | | | | | | | | Suppl./ |
| No. | L⁶ | X¹ | X¹ | m¹ | R² | R³ | A | Ref. |
| Q1-6-1-1 | 5-Cl— | 3- | — | 1 | — | — | —COMe | Wako |
| | | Me- | | | | | | |
| Q1-6-1-2 | 5-Cl— | 3- | — | 1 | — | — | —COOH | Maybridge |
| | | Me- | | | | | | |
| Q1-6-1-3 | 5-Br— | — | — | 1 | — | — | —COOMe | Bionet |
| Q1-6-1-4 | 5-F— | — | — | 1 | — | — | —COOH | Enamine |
| Q1-6-1-5 | 5-Cl— | — | — | 1 | — | — | —COOH | SPECS |
| Q1-6-1-6 | 6-F— | — | — | 1 | — | — | —COOH | Enamine |
| Q1-6-1-7 | 4-Cl— | — | — | 1 | — | — | —COOH | Enamine |
| Q1-6-1-8 | 4-F— | — | — | 1 | — | — | —COOMe | Bionet |
| Q1-6-1-9 | 4-F— | 3- | — | 1 | — | — | —COOH | Enamine |
| | | Me- | | | | | | |
| Q1-6-1-10 | 5-Cl— | — | — | 1 | — | — | —CHO | (ref1) |
| Q1-6-1-11 | 5-F— | — | — | 1 | — | — | —CHO | (ref1) |

(ref1) N. Matsunaga, et al., Bioorg. Med. Chem. 12, 2251 (2004)

TABLE 28

| | |
|---|---|
| | (Q1-6-2) |
| 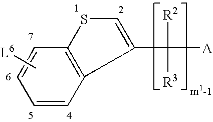 | |

| | | | | | | | | Suppl./ |
|---|---|---|---|---|---|---|---|---|
| No. | L⁶ | X¹ | X¹ | m¹ | R² | R³ | A | Ref. |
| Q1-6-2-1 | 5-Cl— | — | — | 1 | — | — | —CHO | Maybridge |
| Q1-6-2-2 | 6-Br— | — | — | 1 | — | — | —CHO | (ref1) |
| Q1-6-2-3 | 6-Br— | — | — | 1 | — | — | —CHO | (ref1) |

(ref1) S. Mitsumori, et al., J. Med. Chem., 46, 2446 (2003)

TABLE 29

| | | | | | | | | (Q1-7-1) |
|---|---|---|---|---|---|---|---|---|
| 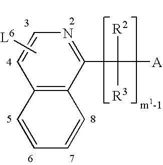 | | | | | | | | |
| | | | | | | | | Suppl./ |
| No. | L⁶ | X¹ | X¹ | m¹ | R² | R³ | A | Ref. |
| Q1-7-1-1 | 5-F— | — | — | 1 | — | — | —CHO | (ref1) |
| Q1-7-1-2 | 5-Cl— | — | — | 1 | — | — | —CHO | (ref1) |

(ref1) French, et al., J. Med. Chem., 13, 1117 (1970)

TABLE 30

| | |
|---|---|
| | (Q1-8-1) |
| 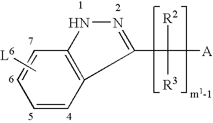 | |

| No. | L⁶ | X¹ | X¹ | m¹ | R² | R³ | A | Suppl./Ref. |
|---|---|---|---|---|---|---|---|---|
| Q1-8-1-1 | 5-F— | — | — | 1 | — | — | —COOH | ChemPacific |
| Q1-8-1-2 | 5-Cl— | — | — | 1 | — | — | —COOH | ChemPacific |
| Q1-8-1-3 | 6-F— | — | — | 1 | — | — | —COOH | ChemPacific |
| Q1-8-1-4 | 6-Br— | — | — | 1 | — | — | —COOH | JWPharmlab |
| Q1-8-1-5 | 5-Br— | — | — | 1 | — | — | —COOEt | JWPharmlab |
| Q1-8-1-6 | 5-I— | — | — | 1 | — | — | —COOH | JWPharmlab |
| Q1-8-1-7 | 5-Cl— | 6- | — | 1 | — | — | —COOH | JWPharmlab |
| | | Cl— | | | | | | |
| Q1-8-1-8 | 7-Cl— | — | — | 1 | — | — | —COOH | JWPharmlab |
| Q1-8-1-9 | 5-F— | 6- | — | 1 | — | — | —COOH | JWPharmlab |
| | | F— | | | | | | |
| Q1-8-1-10 | 6-Cl— | — | — | 1 | — | — | —COOH | JWPharmlab |
| Q1-8-1-11 | 6-Br— | — | — | 1 | — | — | —COOEt | JWPharmlab |
| Q1-8-1-12 | 5-Cl— | — | — | 1 | — | — | —CHO | (ref1) |

(ref1) G. Buechi, et al., J. Am. Chem. Soc., 108, 4115 (1988)

Furthermore, among the compounds represented by general formula (Q1), the compounds represented by general formulas (Q1-5-4-1), (Q1-7-2-1), and (Q1-7-3-1) can be easily produced according to the methods described in, for example, S. R. Inglis, J. Med. Chem., 47, 5405 (2004), M. D. Nair, et al., Indian J. Chem., 10, 341 (1972), and Sing-Yuen Sit, et al., Bioorg. Med. Chem. 12, 715 (2004), respectively.

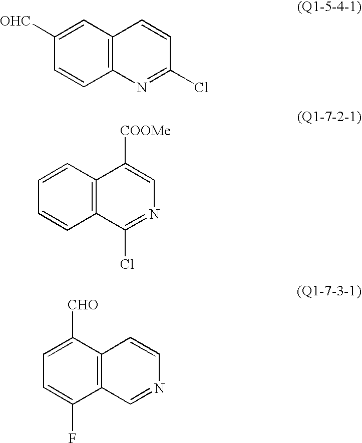

A compound represented by general formula (Q1-9) which is a compound of general formula (Q1) wherein L⁶ is a trifluoromethanesulfonyloxy group can be produced, for example, by the method as shown in the reaction scheme for Production method Q1:

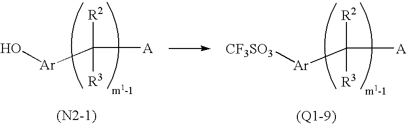

wherein Ar, A, R², R³, and m¹ have the same meanings as the defined above. A compound represented by general formula (Q1-9) can be produced by subjecting the compound represented by general formula (N2-1) to trifluoromethanesulfonylation by means of a trifluoromethanesulfonating agent in an inactive solvent, and if necessary, in the presence of a base. The trifluoromethanesulfonating agent may be exemplified by trifluoromethanesulfonic anhydride, or the like. The base used in the trifluoromethanesulfonation reaction may be exemplified by triethylamine, diisopropylethylamine, pyridine, or the like. The type of the solvent used in the trifluoromethanesulfonation reaction is not particularly limited as long as the solvent is inactive to the reaction. Examples thereof include saturated hydrocarbon solvents, halogenated hydrocarbon solvents, ether solvents, and aromatic hydrocarbon solvents. These solvents may be used individually or as solvent mixtures at any ratio. The saturated hydrocarbon solvent may be exemplified by pentane, hexane, heptane, cyclohexane or the like; and the halogenated hydrocarbon solvent may be exemplified by dichloromethane, chloroform, 1,2-dichloroethane, or the like. The ether solvent may be exemplified by tetrahydrofuran, diethyl ether, 1,4-dioxane, or the like; and the aromatic hydrocarbon solvent may be exemplified by toluene, xylene, or the like. Preferred examples include dichloromethane, chloroform, diethyl ether, tetrahydrofuran, toluene, and the like.

The amount of the trifluoromethanesulfonating agent used in the trifluoromethanesulfonation reaction is preferably 0.5-fold or more the molar amount, and more preferably an equimolar or more amount, of the compound represented by general formula (N2-1). Also, the amount is preferably 10-fold or less the molar amount, and more preferably 2-fold or less the molar amount, of the compound. The amount of the base used in the trifluoromethanesulfonation reaction is preferably an equimolar or more amount, and also preferably 2-fold or less the molar amount, of the trifluoromethanesulfonating agent. The reaction temperature may vary depending on the raw material compound, solvent or the like, but typically, it is preferable to perform the reaction at a temperature within the range of −100° C. to room temperature. The reaction time may vary depending on the raw material compound, solvent, reaction temperature or the like, but typically, the time may be from 1 minute to 12 hours, for example.

Furthermore, in the reaction scheme for Production Method P1, a significant number of the compounds represented by general formula (P2-2) are known, and can be purchased from, for example, the companies described in (Table 31) to (Table 33).

TABLE 31

| | |
|---|---|
| | (P2-2-1-1) |
| 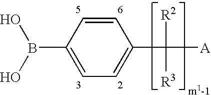 | |

| No. | X¹ | X¹ | m¹ | R² | R³ | A | Suppl./Ref. |
|---|---|---|---|---|---|---|---|
| P2-2-1-1-1 | — | — | 1 | — | — | —COMe | Wako |
| P2-2-1-1-2 | — | — | 1 | — | — | —COOH | TCI |
| P2-2-1-1-3 | — | — | 1 | — | — | —CHO | TCI |
| P2-2-1-1-4 | — | — | 1 | — | — | —COOMe | Wako |
| P2-2-1-1-5 | — | — | 1 | — | — | —COO-t-Bu | Combi-Blocks |
| P2-2-1-1-6 | 2-F— | — | 1 | — | — | —COOH | Combi-Blocks |
| P2-2-1-1-7 | 2-F— | — | 1 | — | — | —CHO | Apollo |
| P2-2-1-1-8 | 2-F— | 3-F— | 1 | — | — | —CHO | Aldrich |
| P2-2-1-1-9 | 2-F— | — | 1 | — | — | —COOMe | Combi-Blocks |
| P2-2-1-1-10 | 2-Cl— | — | 1 | — | — | —COOH | Combi-Blocks |
| P2-2-1-1-11 | 2-Cl— | — | 1 | — | — | —COOMe | Combi-Blocks |
| P2-2-1-1-12 | 2-Cl— | — | 1 | — | — | —COOEt | Combi-Blocks |
| P2-2-1-1-13 | 3-MeO— | 5-MeO— | 1 | — | — | —COOH | RareChemicals |

TABLE 32

| | |
|---|---|
| | (P2-2-2-1) |
| 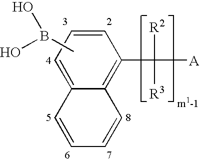 | |

| No. | B(OH)₂-posit. | X¹ | X¹ | m¹ | R² | R³ | A | Suppl./Ref. |
|---|---|---|---|---|---|---|---|---|
| P2-2-2-1-1 | 4- | — | — | 1 | — | — | —CHO | Combi-Blocks |
| P2-2-2-1-2 | 4- | — | — | 1 | — | — | —COOH | RareChemicals |

TABLE 33

| | | | | | | | | (P2-2-3-1) |
|---|---|---|---|---|---|---|---|---|
| 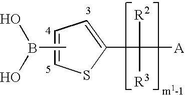 | | | | | | | | |
| | B(OH)₂- | | | | | | | Suppl./ |
| No. | posit. | X¹ | X¹ | m¹ | R² | R³ | A | Ref. |
| P2-2-3-1-1 | 5- | — | — | 1 | — | — | —COMe | Aldrich |
| P2-2-3-1-2 | 5- | — | — | 1 | — | — | —CHO | Aldrich |
| P2-2-3-1-3 | 5- | — | — | 1 | — | — | —COOH | Maybridge |
| P2-2-3-1-4 | 5- | 3- | — | 1 | — | — | —CHO | Frontier |
| | | Me- | | | | | | |
| P2-2-3-1-5 | 4- | — | — | 1 | — | — | —CHO | Frontier |

Furthermore, a compound represented by general formula (P2-2) can be produced by, for example, an established known production method as shown in the reaction scheme for Production Method Q2:

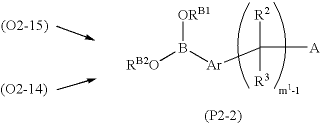

wherein Ar, A, R², R³ and m¹ have the same meanings as the defined above, for example, the method described in [Chemical Review, Vol. 95, 2457 (1995)] or Y. Satoh et al., SYNTHESIS, (1994), 1146, or methods on the basis of the reference literatures described in these literatures. For example, the compound represented by general formula (P2-2) wherein R^{B1} and R^{B2} are each a C1-4 alkyl group can be produced by treating the compound represented by general formula (O2-15) with trialkyl borate. The alkyl group in the trialkyl borate used in the reaction may be exemplified by a C1-C4 alkyl group or the like, and examples thereof include a methyl group, an ethyl group and an isopropyl group. The type of the solvent used in the reaction is not particularly limited as long as the solvent is inactive to the reaction. Examples thereof include saturated hydrocarbon solvents, aromatic hydrocarbon solvents, and ether solvents. The saturated hydrocarbon solvent may be exemplified by pentane, hexane, heptane, cyclohexane, or the like; the aromatic hydrocarbon solvent may be exemplified by toluene, xylene, or the like; and the ether solvent may be exemplified by tetrahydrofuran, diethyl ether, 1,2-dimethoxyethane, 1,4-dioxane, or the like. Pentane, hexane, cyclohexane, toluene, tetrahydrofuran, 1,2-dimethoxyethane, diethyl ether, and solvent mixtures of these solvents at any ratio are preferred. The amount of trialkyl borate used in the reaction is preferably 0.5-fold or more the molar amount, and preferably 1.5-fold or less the molar amount, of the compound represented by general formula (O2-15). The reaction temperature may vary depending on the raw material compound, solvent or the like, but typically, it is preferable to perform the reaction at a temperature within the range of −100° C. to room temperature. The reaction time may vary depending on the raw material compound, solvent, reaction temperature or the like, but typically, the time may be from 10 minutes to 48 hours. As the group A in the compound represented by general formula (O2-15) used in the reaction, the group represented by general formula (2-1A-3) and the like is preferred.

The compound represented by general formula (P2-2) wherein R^{B1} and R^{B2} are each a hydrogen atom can be produced by hydrolyzing a compound which is the compound represented by the general formula (P2-2) in which R^{B1} and R^{B2} are each a C1-4 alkyl group, with a mineral acid. The mineral acid used in the hydrolysis reaction may be exemplified by hydrochloric acid, sulfuric acid, phosphoric acid, or the like. The solvent used in the hydrolysis reaction may be exemplified by the solvents which are the same as the solvents used in the method for producing a compound represented by the general formula (P2-2) in which R^{B1} and R^{B2} are each a C1-4 alkyl group. The reaction temperature may vary depending on the raw material compound, solvent or the like, but typically, it is preferable to perform the reaction at a temperature within the range of −20° C. to room temperature. The reaction time may vary depending on the raw material compound, solvent, reaction temperature or the like, but typically, the time may be, for example, from 10 minutes to 24 hours.

Many of the compounds represented by general formula (P2-2) in which R^{B1} and R^{B2} are joined together to form a 1,1,2,2-tetra methylethylene group are commercially available and can be easily purchased. Also, the compound can be produced by allowing the compound represented by general formula (O2-14) to react with a boron compound in the presence of a palladium catalyst. The palladium catalyst used in the reaction may be exemplified by tetrakis(triphenylphosphine)palladium, tetrakis(methyldiphenylphosphine)palladium, dichlorobis(triphenylphosphine)palladium, dichlorobis(tri-o-tolylphosphine)palladium, dichlorobis(tricyclohexylphosphine)palladium, dichlorobis(triethylphosphine)palladium, palladium acetate, palladium chloride, bis(acetonitrile)palladium chloride, tris(dibenzylideneacetone)dipalladium, bis(diphenylphosphinoferrocene)palladium chloride, or the like. Furthermore, a catalyst prepared from palladium acetate or tris(dibenzylideneacetone) dipalladium and an arbitrary ligand, can also be used. The valence of palladium may be zero or +2. The ligand for palladium may be exemplified by phosphine ligands such as trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, or 2-(di-t-butylphosphino) biphenyl; or non-phosphine ligands such as imidazol-2-ylidenecarbenes.

The amount of the palladium catalyst used in the reaction is preferably 0.01 to 20% by mole, and more preferably 0.1 to 10% by mole.

The boron compound used in the reaction may be exemplified by 4,4,5,5-tetra methyl-1,3,2-dioxaborolane, bis(pinacolato)diboron, or the like.

The base used in the reaction may be exemplified by sodium carbonate, potassium carbonate, cesium carbonate, cesium fluoride, potassium fluoride, potassium phosphate, potassium acetate, triethylamine, potassium hydroxide, sodium hydroxide, sodium methoxide, lithium methoxide, or the like.

The solvent used in the reaction is not particularly limited as long as the solvent is inactive to the reaction. Examples thereof include hydrocarbon solvents such as toluene, xylene, or hexane; halogen hydrocarbon solvents such as dichloromethane or chloroform; sulfoxide solvents such as dimethylsulfoxide; amide solvents such as dimethylformamide; ether solvents such as tetrahydrofuran, dioxane or diglyme; alcohol solvents such as methanol or ethanol; nitrile solvents such as acetonitrile; ketone solvents such as acetone or cyclohexanone; ester solvents such as ethyl acetate; or heterocyclic solvents such as pyridine. Also, two or more organic solvents may be used as mixtures. Furthermore, the solvent system may be any of a biphasic system of water-organic solvent, a water-containing organic solvent, and a homogeneous system of an organic solvent or solvents.

The reaction temperature may vary depending on the type of the raw material compound, catalyst, base, solvent or the like, but typically, the temperature may be, for example, from 0° C. to 150° C., and is preferably from room temperature to 120° C. The reaction time may vary depending on the raw material compound, catalyst, base, solvent, reaction temperature or the like, but typically, the time may be, for example, from 30 minutes to 72 hours, and is preferably from 1 hour to 48 hours.

In the reaction schemes for Production Methods N6 and N7, a significant number of the compounds represented by general formula (N2-25) and general formula (N2-29) are known, and the compounds may be commercially available or can be easily produced by an established known method, for example, the process as shown in the reaction scheme for Production Method Q3:

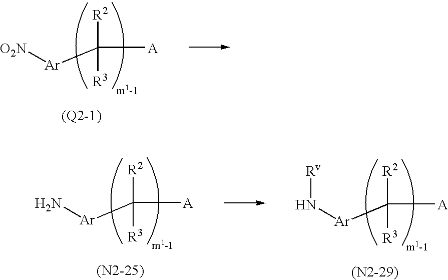

wherein Ar, A, R², R³, R^{V} and m¹ have the same meanings as the defined above.

The compound represented by general formula (N2-25) can be produced by reducing a compound represented by general formula (Q2-1) which is commercially available, or can be produced according to a known method or a method equivalent thereto. The reduction reaction used for the reduction reaction may be exemplified by a method for catalytic reduction in a hydrogen atmosphere, or the like. For the method for catalytic reduction reaction, there may be listed a method similar to the method for producing the compound represented by general formula (O2-7) in which V is —CH₂CH₂—, by reducing (preferably, catalytic reduction in a hydrogen atmosphere) the compound represented by general formula (O2-6). As the group A in the compound represented by general formula (Q2-1) used in the catalytic reduction reaction, the group represented by general formula (2-1A-4), (2-1A-5) or (2-1A-6) is preferred.

Furthermore, in another method, the compound represented by general formula (N2-25) can be produced by reducing a compound represented by general formula (Q2-1) using sodium borohydride in the presence of a transition metal catalyst. The solvent used in the reduction reaction may be exemplified by alcohol solvents, or the like, and if necessary, ether solvents and the like may be favorably added thereto. The alcohol solvent may be exemplified by methanol, ethanol or the like, and the ether solvent which may be added as necessary, may be exemplified by tetrahydrofuran, 1,4-dioxane, or the like. The transition metal catalyst used in the reduction reaction may be exemplified by nickel chloride, cobalt chloride, or hydrates thereof. The amount of sodium borohydride used in the reduction reaction may be from 0.5-fold to 5-fold the molar amount of the compound represented by general formula (Q2-1). The amount of the transition metal catalyst used in the reduction reaction may be from 1% by mole to 20% by mole relative to the compound represented by general formula (Q2-1). The reaction temperature may vary depending on the raw material compound, solvent, catalyst or the like, but typically, the temperature may be from −2° C. to ambient temperature, for example. The reaction time may vary depending on the raw material compound, solvent, catalyst, reaction time or the like, but typically, the time may be, for example, from 1 minute to 12 hours. As the group A in the compound represented by general formula (Q2-1) used in the reduction reaction, the group represented by general formula (2-1A-3), (2-1A-4), (2-1A-5) or (2-1A-6) is preferred. If the group A is a group represented by general formula (2-1A-1), it is preferable to convert the group A to the group represented by general formula (2-1A-3) by acetalizing the group according to a known method, for example, the method described in Protective Groups in Organic Synthesis, published by John Wiley and Sons (1999), and then supplying the compound to the reduction reaction.

In the reaction scheme for Production Method Q3, the compound represented by general formula (Q2-1) can be, for example, purchased from the companies described in (Table 34) to (Table 45), or easily produced according to the literatures described in the footnotes of (Table 34) to (Table 45).

TABLE 34

| | |
|---|---|
| | (Q2-1-1-1) |
| 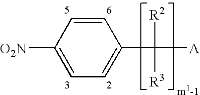 | |

| No. | X¹ | X¹ | m¹ | R² | R³ | A | Suppl./Ref. |
|---|---|---|---|---|---|---|---|
| Q2-1-1-1-1 | — | — | 1 | — | — | —CHO | TCI |
| Q2-1-1-1-2 | 2-MeO— | — | 1 | — | — | —CHO | AstaTech |
| Q2-1-1-1-3 | 3-OH— | — | 1 | — | — | —CHO | Aldrich |
| Q2-1-1-1-4 | — | — | 1 | — | — | —COMe | TCI |
| Q2-1-1-1-5 | 3-MeO— | — | 1 | — | — | —COMe | G&J |
| Q2-1-1-1-6 | — | — | 1 | — | — | —COOEt | TCI |
| Q2-1-1-1-7 | 3-Me- | — | 1 | — | — | —COOEt | Wako |
| Q2-1-1-1-8 | 2-Cl— | — | 1 | — | — | —COOMe | Wako |
| Q2-1-1-1-9 | 3-MeO— | — | 1 | — | — | —COOMe | Wako |
| Q2-1-1-1-10 | 2-F— | — | 1 | — | — | —COOt-Bu | Asymchem |
| Q2-1-1-1-11 | — | — | 1 | — | — | —COOt-Bu | Buttpark |
| Q2-1-1-1-12 | — | — | 2 | H— | H— | —COOH | TCI |
| Q2-1-1-1-13 | — | — | 2 | H— | H— | —COOMe | SALOR |
| Q2-1-1-1-14 | — | — | 2 | H— | H— | —COOEt | TCI |
| Q2-1-1-1-15 | — | — | 2 | H— | H— | —COMe | Wako |
| Q2-1-1-1-16 | — | — | 2 | Me- | H— | —COOH | Aldrich |
| Q2-1-1-1-17 | 3-Cl— | — | 2 | H— | H— | —COOH | Tyger |
| Q2-1-1-1-18 | 3-F— | — | 2 | H— | H— | —COOH | Apollo |

TABLE 35

| | | | | | | | | (Q2-1-2-1) |
|---|---|---|---|---|---|---|---|---|
| 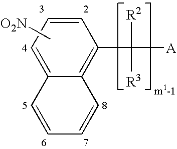 | | | | | | | | |
| | NO₂- | | | | | | | Suppl./ |
| No. | posit. | X¹ | X¹ | m¹ | R² | R³ | A | Ref. |
| Q2-1-2-1-1 | 4- | — | — | 1 | — | — | —COMe | (ref1) |
| Q2-1-2-1-2 | 4- | — | — | 1 | — | — | —CHO | (ref2) |

(ref1) E.. Paredes, et al., Tetrahedron Lett., 43, 4601 (2002)
(ref2) A. R. Katritzky, et al., Tetrahedron Lett., 37, 347 (1996)

TABLE 36

| | |
|---|---|
| | (Q2-1-2-2) |
| 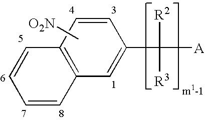 | |

| | NO₂- | | | | | | | Suppl./ |
|---|---|---|---|---|---|---|---|---|
| No. | posit. | X¹ | X¹ | m¹ | R² | R³ | A | Ref. |
| Q2-1-2-2-1 | 6- | — | — | 1 | — | — | —COOH | (ref1) |
| Q2-1-2-2-2 | 6- | — | — | 1 | — | — | —COOMe | (ref1) |
| Q2-1-2-2-3 | 6- | 4-OH— | — | 1 | — | — | —COOEt | (ref2) |
| Q2-1-2-2-4 | 6- | 4-OH— | — | 1 | — | — | —COMe | (ref3) |

(ref1) W. Adcock, et al., Aust. J. Chem., 18, 1351 (1965)
(ref2) G. Jia. et al., Bioorg. Med. Chem., 8, 1607 (2000)
(ref3) R. Sen., J. Ind. Chem. Soc., 7, 401 (1930)

TABLE 37

| | | | | | | | | (Q2-1-3-1) |
|---|---|---|---|---|---|---|---|---|
| 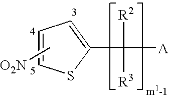 | | | | | | | | |
| | NO₂- | | | | | | | Suppl./ |
| No. | posit. | X¹ | X¹ | m¹ | R² | R³ | A | Ref. |
| Q2-1-3-1-1 | 5- | — | — | 1 | — | — | —CHO | Wako |
| Q2-1-3-1-2 | 5- | — | — | 1 | — | — | —COOH | Wako |
| Q2-1-3-1-3 | 5- | — | — | 1 | — | — | —COMe | Lancaster |
| Q2-1-3-1-4 | 4- | 5-Cl— | — | 1 | — | — | —COMe | Maybridge |
| Q2-1-3-1-5 | 4- | 5-Et- | — | 1 | — | — | —CHO | Princeton |
| Q2-1-3-1-6 | 4- | 5-Me- | — | 1 | — | — | —CHO | Princeton |

TABLE 38

| | | | | | | | | (Q2-1-3-2) |
|---|---|---|---|---|---|---|---|---|
| 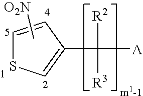 | | | | | | | | |
| | | | | | | | | Suppl./ |
| No. | NO₂-posit. | X¹ | X¹ | m¹ | R² | R³ | A | Ref. |
| Q2-1-3-2-1 | 5- | — | — | 1 | — | — | —COOH | Alfa Aesar |
| Q2-1-3-2-2 | 5- | — | — | 1 | — | — | —CHO | Wako |
| Q2-1-3-2-3 | 5- | — | — | 1 | — | — | —COMe | Maybridge |

TABLE 39

| | | | | | | | | (Q2-1-4-1) |
|---|---|---|---|---|---|---|---|---|
| 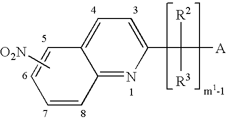 | | | | | | | | |
| | NO₂- | | | | | | | Suppl./ |
| No. | posit. | X¹ | X¹ | m¹ | R² | R³ | A | Ref. |
| Q2-1-4-1-1 | 6- | 4- | — | 1 | — | — | —COOMe | Maybridge |
| | | Cl— | | | | | | |
| Q2-1-4-1-2 | 5- | — | — | 1 | — | — | —COOH | SALOR |
| Q2-1-4-1-3 | 5- | — | — | 1 | — | — | —CHO | OTAVA |

TABLE 40

| | | | | | | | | (Q2-1-4-2) |
|---|---|---|---|---|---|---|---|---|
| 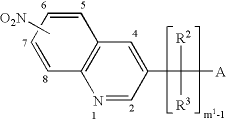 | | | | | | | | |
| | NO₂- | | | | | | | Suppl./ |
| No. | posit. | X¹ | X¹ | m¹ | R² | R³ | A | Ref. |
| Q2-1-4-2-1 | 6- | 2-OH— | — | 1 | — | — | —COOH | AstaTech |
| Q2-1-4-2-2 | 7- | 2-Me- | — | 1 | — | — | —COOEt | (ref1) |
| Q2-1-4-2-3 | 7- | 4-Cl— | — | 1 | — | — | —COOEt | (ref2) |
| Q2-1-4-2-4 | 7- | 2-Cl— | — | 1 | — | — | —CHO | (ref3) |
| Q2-1-4-2-5 | 7- | 4-OH— | — | 1 | — | — | —COOEt | (ref4) |

(ref1) D. R. Adams, Tetrahedron Lett., 24, 517 (1983)
(ref2) US3362954
(ref3) M. M. Ali, et al., Synth. Commun., 32, 1351 (2002)
(ref4) S. R. Vippagunta, et al., J. Med. Chem., 42, 4630 (1999)

TABLE 41

| | |
|---|---|
| | (Q2-1-4-3) |
| 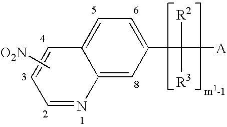 | |

| | NO₂- | | | | | | | Suppl./ |
|---|---|---|---|---|---|---|---|---|
| No. | posit. | X¹ | X¹ | m¹ | R² | R³ | A | Ref. |
| Q2-1-4-3-1 | 3- | 4-Me- | — | 1 | — | — | —COMe | (ref1) |
| Q2-1-4-3-2 | 3- | 4-OH— | — | 1 | — | — | —COOMe | (ref2) |

(ref1) Schofield, et al., J. Chem. Soc., 1950, 395
(ref2) Sues, et al., Justus Liebigs Ann. Chem., 593, 91 (1955)

TABLE 42

| | |
|---|---|
| | (Q2-1-4-4) |
| 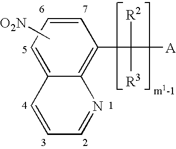 | |

| | NO₂- | | | | | | | |
|---|---|---|---|---|---|---|---|---|
| No. | posit. | X¹ | X¹ | m¹ | R² | R³ | A | Suppl./Ref. |
| Q2-1-4-4-1 | 5- | — | — | 1 | — | — | —CHO | (ref1) |
| Q2-1-4-4-2 | 5- | — | — | 1 | — | — | —COOH | (ref2) |
| Q2-1-4-4-3 | 5- | 2-CF₃— | 3-Et- | 1 | — | — | —COOH | (ref3) |
| Q2-1-4-4-4 | 5- | 6-Cl— | — | 1 | — | — | —COOMe | (ref4) |

(ref1) Howitz, et al., Chem. Ber., 39, 2708 (1906)
(ref2) Bradford, et al., J. Chem. Soc., 1947, 437
(ref3) Steglich, et al., Angew. Chem. 85, 505 (1973)
(ref4) Bailey, et al., J. Med. Chem. 13, 598 (1970)

TABLE 43

| | |
|---|---|
| | (Q2-1-5-1) |
| 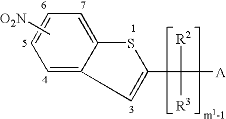 | |

| No. | NO₂-posit. | X¹ | X¹ | m¹ | R² | R³ | A | Suppl./Ref. |
|---|---|---|---|---|---|---|---|---|
| Q2-1-5-1-1 | 5- | 3-Me- | — | 1 | — | — | —COOEt | FluoroChem |
| Q2-1-5-1-2 | 5- | — | — | 1 | — | — | —COOH | Maybridge |
| Q2-1-5-1-3 | 5- | — | — | 1 | — | — | —COOEt | Bionet |
| Q2-1-5-1-4 | 5- | — | — | 1 | — | — | —COOMe | Buttpark |
| Q2-1-5-1-5 | 5- | 3-Me- | — | 1 | — | — | —COOH | SPECS |
| Q2-1-5-1-6 | 6- | 3-Cl— | — | 1 | — | — | —COOH | Oakwood |
| Q2-1-5-1-7 | 4- | 3-Cl— | — | 1 | — | — | —COOH | ART-Chem |

TABLE 44

| | | | | | | | | (Q2-1-5-2) |
|---|---|---|---|---|---|---|---|---|
| 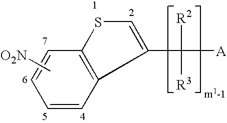 | | | | | | | | |
| | | | | | | | | Suppl./ |
| No. | NO₂-posit. | X¹ | X¹ | m¹ | R² | R³ | A | Ref. |
| Q2-1-5-2-1 | 6- | — | — | 1 | — | — | —COMe | (ref1) |
| Q2-1-5-2-2 | 6- | — | — | 1 | — | — | —COMe | (ref1) |

(ref1) Brophy, et al., J. Chem. Soc. C, 1970, 933

TABLE 45

| | |
|---|---|
| | (Q2-1-6-1) |
| 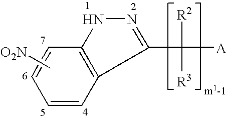 | |

| No. | NO₂-posit. | X¹ | X¹ | m¹ | R² | R³ | A | Suppl./Ref. |
|---|---|---|---|---|---|---|---|---|
| Q2-1-6-1-1 | 5- | — | — | 1 | — | — | —COOH | JWPharmlab |
| Q2-1-6-1-2 | 6- | — | — | 1 | — | — | —CHO | (ref1) |
| Q2-1-6-1-3 | 6- | — | — | 1 | — | — | —COOEt | (ref2) |
| Q2-1-6-1-4 | 6- | 1-Me- | — | 1 | — | — | —COOEt | (ref2) |
| Q2-1-6-1-5 | 6- | 1-Ph- | — | 1 | — | — | —COOEt | (ref3) |
| Q2-1-6-1-6 | 6- | 1-Ph- | — | 1 | — | — | —COMe | (ref4) |

(ref1) Han-Cheng Zhang et al., J. Med Chem., 44, 1021 (2001)
(ref2) Hahn, et al., Chem. Ber., 65, 717 (1932)
(ref3) Strassmann, Chem. Ber., 23, 715 (1890)
(ref4) Borsche, Chem. Ber., 42, 608 (1909)

Furthermore, among the compounds represented by general formula (Q2-1), the compounds represented by general formulas (Q21-7-1-1) and (Q2-1-7-2-l) can be purchased from, for example, SynChem, Inc. or the like, and the compounds represented by general formulas (Q2-1-4-5-1) and (Q2-1-8-1-1) can be easily produced according to the methods described in, for example, R. Kaslow, J. Am. Chem. Soc., 70, 3912 (1948), and M. Sugiura, et al., Chem. Pharm. Bull., 40, 2262 (1992), respectively.

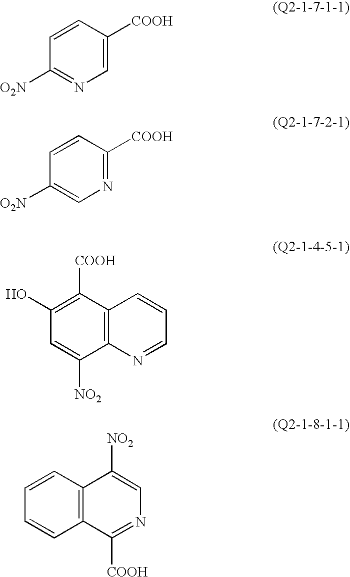

Meanwhile, the compound represented by general formula (N2-29) can be produced through a reductive amination reaction of the compound represented by general formula (N2-25), or the like. For the reductive amination reaction, there may be listed a method which is the same as the method for producing a compound represented by general formula (2A) of the reaction scheme for the Production Method A in which R¹ is a C1-4 alkyl group, through a reductive amination reaction between a compound represented by general formula (2A) in which R¹ is a hydrogen atom and a known C1-4 saturated aldehyde or C1-4 saturated ketone.

Furthermore, in another method, the compound represented by general formula (N2-29) can be produced through an alkylation reaction of the compound represented by general formula (N2-25), or the like. For the alkylation reaction, there may be listed a method which is the same as the method for producing a compound represented by the general formula (2A) in which R¹ is a C1-4 alkyl group, through an alkylation reaction between a compound represented by general formula (2A) in which R¹ is a hydrogen atom, and a known compound represented by R¹L¹ (wherein R¹ represents a C1-4 alkyl group; and L¹ represents a leaving group)

Furthermore, a significant number of the compounds represented by general formulas (N2-31) and (P2-5) in the respective reaction schemes for Production Methods N7 and P2 are known, and the compound may be commercially available, or can be easily produced by established known methods, for example, the reaction scheme for Production Method Q4:

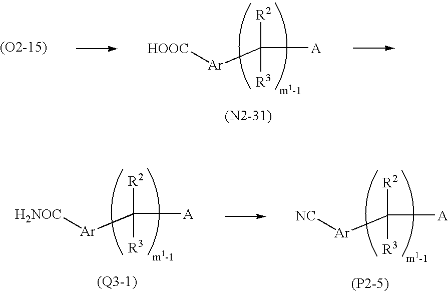

wherein Ar, A, R², R³ and m¹ have the same meanings as the defined above.

A compound represented by general formula (N2-31) can be produced by treating the compound represented by general formula (O2-15) with carbon dioxide. The type of the solvent used in the carboxylation reaction is not particularly limited as long as the solvent is inactive to the reaction. Examples thereof include the solvents which are the same as the solvents exemplified for the reaction of formylating a lithio product of the compound represented by general formula (2-1-2). The state of the carbon dioxide used in the carboxylation reaction may be solid, gas or the like. The amount of carbon dioxide used in the carboxylation reaction may be 0.5-fold or more the molar amount, and preferably an equimolar amount, of the compound represented by general formula (O2-15). The reaction temperature may vary depending on the raw material compound, solvent or the like, but typically, a temperature from −100° C. to ambient temperature may be used. The reaction time may vary depending on the raw material compound, solvent, reaction temperature or the like, but typically, the time may be from 10 minutes to 6 hours. As the group A in the compound represented by general formula (O2-15), which is used in the carboxylation reaction, the group represented by general formula (2-1A-3) or the like is preferred.

The compound represented by general formula (Q3-l) can be produced by subjecting the compound represented by general formula (N2-31) and ammonia to a condensation reaction in the presence of a dehydrating-condensing agent. The state of ammonia used in the condensation reaction may be exemplified by solid or solution. The type of the solvent used in the solution is not particularly limited as long as the solvent is inactive to the reaction. Examples thereof include water, or the solvents the same as the solvents used in the reaction. For the condensation reaction, there may be listed a method which is the same as the method for producing the compound represented by general formula (N2-30) by subjecting the compound represented by general formula (N2-28) and the compound represented by general formula (N2-29) to a condensation reaction in the presence of a dehydrating-condensing agent.

Furthermore, in another production method, the compound represented by general formula (Q3-1) can also be produced by reacting a reactive derivative of the compound represented by general formula (N2-31) with ammonia in an inactive solvent. For the reaction, there may be listed a method which is the same as the method for producing the compound represented by general formula (N2-30) by allowing a reactive derivative of the compound represented by general formula (N²-28) to react with the compound represented by general formula (N2-29) in an inactive solvent. The compound represented by general formula (P2-5) can be produced by dehydrating the compound represented by general formula (Q3-1) in the presence of a dehydrating agent, and if necessary, in the presence of a base. The dehydration reaction can be performed in the absence of solvent or in the presence of solvent. The type of the solvent used in the dehydration reaction is not particularly limited as long as the solvent is inactive to the reaction. Examples thereof include saturated hydrocarbon solvents, halogenated hydrocarbon solvents, ether solvents, nitrile solvents, amide solvents, or the like. The solvent is, for example, hexane, dichloromethane, chloroform, diethyl ether, tetrahydrofuran, 1,4-dioxane, acetonitrile, N,N-dimethylformamide. The dehydrating agent used in the dehydration reaction may be exemplified by phosphorus pentaoxide, phosphorus pentachloride, thionyl chloride, para-toluenesulfonyl chloride, methanesulfonyl chloride, trifluoromethanesulfonic anhydride, pivaloyl chloride, N,N′-dicyclohexylcarbodiimide, triphenylphosphine/carbon tetrachloride, triphenylphosphine/triphosgene, triphenylphosphine/N-chlorosuccinimide, cyanuric acid chloride, or the like. The amount of the dehydrating agent used in the dehydration reaction is preferably 0.1-fold or more the molar amount, and preferably 10-fold or less the molar amount, of the compound represented by general formula (Q3-1). The base used in the reaction as necessary may be exemplified by triethylamine, diisopropylethylamine, pyridine, or the like. The amount of the base used in the reaction as necessary is preferably 0.5-fold or more the molar amount, and preferably 10-fold or less the molar amount, of the dehydrating agent. The reaction temperature may vary depending on the raw material compound, dehydrating agent, solvent or the like, but the temperature may be, for example, from −100° C. to the reflux temperature of the solvent. The reaction time may vary depending on the raw material compound, dehydrating agent, solvent, reaction temperature or the like, but the time may be from 5 minutes to 48 hours, for example.

In the reaction schemes for Production Methods O3 and N4, a significant number of the compounds represented by general formulas (O2-10) and (N2-19) are known, and the compounds may be commercially available or can be easily produced by established known methods, for example, the method shown in the reaction scheme for Production Method Q5:

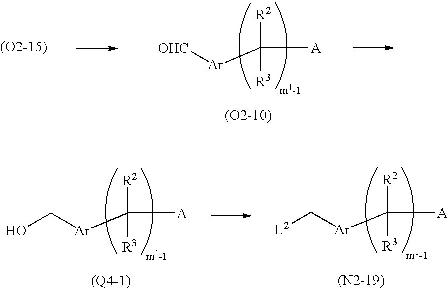

wherein Ar, A, R², R³, L² and m¹ have the same meanings as the defined above.

The compound represented by general formula (O2-10) can be produced by treating a compound represented by general formula (O2-15) with N,N-dimethylformamide. For the formylation reaction, a method which is the same as the reaction for formylating a lithio product of the compound represented by the general formula (2-1-2), may be listed. As the group A in the compound represented by general formula (O2-15) used in the formylation reaction, the group represented by general formula (2-1A-3), and the like are preferred.

The compound represented by general formula (Q4-1) can be produced by reducing the compound represented by general formula (O2-10) with sodium borohydride. The solvent used in the reduction reaction may be exemplified by alcohol solvents, or the like, and if necessary, ether solvents and the like may be added thereto. The alcohol solvent may be exemplified by methanol, ethanol or the like; and the ether solvent which may be added as necessary may be exemplified by tetrahydrofuran, 1,4-dioxane or the like. The amount of sodium borohydride used in the reduction reaction may be, for example, 0.5-fold to 5-fold the molar amount of the compound represented by general formula (O2-10). The reaction temperature may vary depending on the raw material compound, solvent or the like, but typically, the temperature may be from −20° C. to ambient temperature. The reaction time may vary depending on the raw material compound, solvent, reaction time or the like, but typically, the time may be from 1 minute to 6 hours.

With regard to the compound represented by general formula (N2-19), when L² is an acyloxy group, the compound represented by general formula (N2-19) can be produced, for example, by treating a compound represented by general formula (Q4-1) with an acylating agent such as an acyl halide, an acid anhydride or the like, in the presence of a base as necessary. The acyl halide used in the acylation reaction may be exemplified by methanesulfonyl chloride, benzenesulfonyl chloride, para-toluenesulfonyl chloride or the like. The acid anhydride used in the acylation reaction may be exemplified by trifluoromethanesulfonic anhydride or the like. The base used in the acylation reaction may be exemplified by triethylamine, diisopropylethylamine, pyridine or the like. The type of the solvent used in the acylation reaction is not particularly limited as long as the solvent is inactive to the acylation reaction. Examples thereof include saturated hydrocarbon solvents, halogenated hydrocarbon solvents, ether solvents, or aromatic hydrocarbon solvents, and these solvents may be used individually or as solvent mixtures at any ratio. The saturated hydrocarbon solvent may be exemplified by pentane, hexane, heptane, cyclohexane, or the like; and the halogenated hydrocarbon solvent may be exemplified by dichloromethane, chloroform, 1,2-dichloroethane, or the like. The ether solvent may be exemplified by tetrahydrofuran, diethyl ether, 1,4-dioxane, or the like; and the aromatic hydrocarbon solvent may be exemplified by toluene, xylene, or the like. Preferred examples include dichloromethane, chloroform, diethyl ether, tetrahydrofuran, or toluene.

The amount of the acylating agent used in the acylation reaction is preferably 0.5-fold or more the molar amount, and more preferably an equimolar or more amount, of the compound represented by general formula (Q4-1). Also, the amount is preferably 10-fold or less the molar amount, and more preferably 2-fold or less the molar amount, of the compound. The amount of the base used in the acylation reaction is preferably an equimolar or more amount, and preferably 2-fold or less the molar amount, of the acylating agent. The reaction temperature may vary depending on the raw material compound, solvent or the like, but typically, it is preferable to perform the reaction at a temperature within the range of −100° C. to room temperature. The reaction time may vary depending on the raw material compound, solvent, reaction temperature or the like, but typically, the time may be from 1 minute to 12 hours.

With regard to the compound represented by general formula (N2-19), when L is a halogen atom, for example, the compound represented by general formula (N2-19) can be produced by treating a compound represented by general formula (N2-19) in which L² is an acyloxy group with a metal halide or the like. The type of the solvent used in the reaction is not particularly limited as long as the solvent is inactive to the reaction. Examples thereof include alcohol solvents, ether solvents, ketone solvents, and amide solvents, and sulfoxide solvents. The alcohol solvent may be exemplified by methanol, ethanol, n-propanol, 2-propanol or the like. The ether solvent may be exemplified by diethyl ether, tetrahydrofuran, 1,4-dioxane, 1,2-dimethoxyethane or the like. The ketone solvent may be exemplified by acetone or the like. The amide solvent may be exemplified by N,N-dimethylformamide, N,N-dimethylacetamide, N-methylpyrrolidone, or the like. The sulfoxide solvent may be exemplified by dimethylsulfoxide or the like. The metal halide used in the reaction may be exemplified by an alkali metal halide or an alkaline earth metal halide, and lithium chloride, sodium chloride, potassium chloride, magnesium chloride, calcium chloride, lithium bromide, sodium bromide, potassium bromide, magnesium bromide, calcium bromide, lithium iodide, sodium iodide, potassium iodide, magnesium iodide, calcium iodide and the like are preferred. The amount of the metal halide used in the reaction is preferably an equimolar or more amount, and preferably 100-fold or less the molar amount, of the compound represented by general formula (N2-19) in which L² is an acyloxy group. The reaction temperature may vary depending on the raw material compound, solvent or the like, but typically, the temperature may be, for example, from ambient temperature to the reflux temperature of the solvent. The reaction time may vary depending on the raw material compound, solvent, reaction temperature or the like, but typically, the time may be from 1 hour to 48 hours.

In the reaction scheme for Production Method N3, a significant number of the compounds represented by general formula (N2-12) are known, and the compounds may be commercially available or can be easily produced by established known methods, for example, the method shown in the reaction scheme for Production Method Q6:

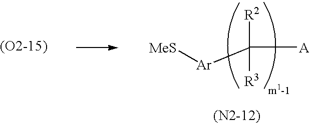

wherein Ar, A, R², R³ and m¹ have the same meanings as the defined above.

The compound represented by general formula (N2-12) can be produced by methylthiolating the compound represented by general formula (O2-15) by means of a methylthiolating agent. The type of the solvent used in the methylthiolation reaction is not particularly limited as long as the solvent is inactive to the reaction. The solvent is, for example, the solvent which is the same as those exemplified for the formylation reaction of a lithio product of the compound represented by the general formula (2-1-2). The methylthiolating agent used in the methylthiolation reaction may be exemplified by S-methylmethanesulfonothioate or the like. The amount of the methylthiolating agent used in the methylthiolation reaction is preferably 0.5-fold or more the molar amount, and preferably 5-fold or less the molar amount, of the compound represented by general formula (O2-15). The reaction temperature may vary depending on the raw material compound, solvent or the like, but typically, the temperature may be from −100° C. to ambient temperature. The reaction time may vary depending on the raw material compound, solvent, reaction temperature or the like, but typically, the time may be from 5 minutes to 12 hours. As the group A in the compound represented by general formula (O2-15) which is used in the methylthiolation reaction, the group represented by general formula (2-1A-3) and the like are preferred.

Also, a significant number of the compounds represented by general formula (P2-8) in the reaction scheme for Production Method P3 are known, and the compounds may be commercially available or can be easily produced by established known methods, for example, the method shown in the reaction scheme for Production Method Q7:

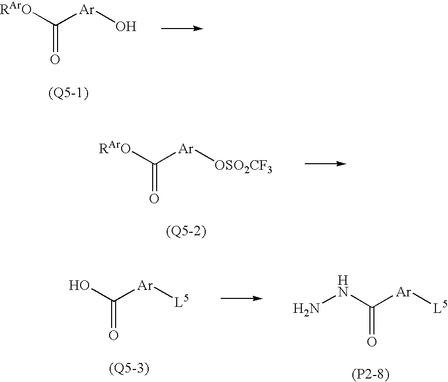

wherein Ar and L⁵ have the same meanings as the defined above; and R^{Ar} represents a C1-4 alkyl group.

A compound represented by general formula (P2-8) in which L⁵ is a chlorine atom, a bromine atom or an iodine atom, can be produced, for example, by subjecting a compound which is the compound represented by general formula (Q5-3) in which L⁵ is a chlorine atom, a bromine atom or an iodine atom, which is a commercially available product, or which can be produced by an established known method or a method equivalent to a known method, and hydrazine to a condensation reaction in the presence of a dehydrating-condensing agent. For the condensation reaction, there may be listed a method which is the same as the method for producing the compound represented by general formula (N2-30) by subjecting the compound represented by general formula (N2-28) and the compound represented by general formula (N2-29) to a condensation reaction in the presence of a dehydrating-condensing agent.

Furthermore, in another production method, the compound represented by general formula (P2-8) in which L⁵ is a chlorine atom, a bromine atom or an iodine atom, can be produced by allowing a reactive derivative of a compound represented by general formula (Q5-3) in which L⁵ is a chlorine atom, a bromine atom or an iodine atom, and hydrazine to react with each other in an inactive solvent. For the reaction, there may be listed a method which is the same as the method for producing the compound represented by general formula (N2-30) by allowing a reactive derivative of the compound represented by general formula (N2-28), and the compound represented by general formula (N2-29) to react with each other in an inactive solvent.

The compound represented by general formula (Q5-2) can be produced by trifluoromethanesulfonylating a compound represented by general formula (Q5-l). For the trifluoromethanesulfonylation reaction, there may be listed a method which is the same as the method for producing the compound represented by general formula (Q1-9) by trifluoromethanesulfonylating the compound represented by general formula (N2-1) in an inactive solvent, in the presence of a base as necessary, using a trifluoromethanesulfonylating agent.

A compound represented by general formula (Q5-3) in which L⁵ is a trifluoromethanesulfonyloxy group, can be produced by converting R^{Ar} of the compound represented by general formula (Q5-2) to a hydrogen atom by a known method, for example, the method described in Protective Groups in Organic Synthesis, published by John Wiley and Sons (1999), or the like. R^{Ar} may be exemplified by a methyl group, ethyl group, a tert-butyl group or the like.

A compound represented by general formula (P2-8) in which L⁵ is a trifluoromethanesulfonyloxy group, can be produced by subjecting a compound represented by general formula (Q5-3) in which L⁵ is a trifluoromethanesulfonyloxy group, and hydrazine to a condensation reaction in the presence of a dehydrating-condensing agent. For the condensation reaction, there may be listed a method which is the same as the method for producing the compound represented by general formula (N2-30) by subjecting the compound represented by general formula (N2-28) and the compound represented by general formula (N2-29) to a condensation reaction in the presence of a dehydrating-condensing agent.

Furthermore, in another production method, a compound represented by general formula (P2-8) in which L⁵ is a trifluoromethanesulfonyloxy group, can also be produced by allowing a reactive derivative of the compound represented by general formula (Q5-3) in which L⁵ is a trifluoromethanesulfonyloxy group to react with hydrazine in an inactive solvent. For the reaction, there may be listed a method which is the same as the method for producing the compound represented by general formula (N2-30) by allowing a reactive derivative of the compound represented by general formula (N2-28) and the compound represented by general formula (N2-29) to react with each other in an inactive solvent.

The compound represented by general formula (Q5-1) can be purchased from, for example, the companies described in (Table 46) to (Table 53), or can be easily produced according to the literatures described in the footnotes of (Table 46) to (Table 53).

TABLE 46

| | |
|---|---|
| | (Q5-1-1-1) |
| 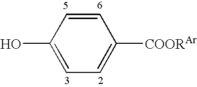 | |

| No. | X¹ | X¹ | R^{hr} | Suppl./Ref. |
|---|---|---|---|---|
| Q5-1-1-1-1 | — | — | -Me | TCI |
| Q5-1-1-1-2 | 3-MeO— | — | -Me | Wako |
| Q5-1-1-1-3 | 3-Cl— | — | -Me | Wako |
| Q5-1-1-1-4 | 3-Cl— | 5-Cl— | -Me | Wako |
| Q5-1-1-1-5 | 3-F— | — | -Me | ChemPacific |

TABLE 47

| | |
|---|---|
| | (Q5-1-2-1) |
| 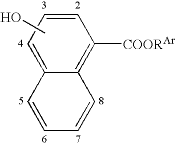 | |

| No. | HO-posit. | X¹ | X¹ | R^{Ar} | Suppl./Ref. |
|---|---|---|---|---|---|
| Q5-1-2-1-1 | 4- | — | — | -Me | (ref1) |
| Q5-1-2-1-2 | 5- | — | — | -Me | (ref1) |
| Q5-1-2-1-3 | 6- | — | — | -Me | (ref2) |

(ref1) J. A. O'Meara, et al., J. Med. Chem., 48, 5580 (2005)
(ref2) P. R. Bernstein, et al., Bioorg. Med. Chem. Lett., 11, 2769 (2001)

TABLE 48

| | |
|---|---|
| | (Q5-1-2-2) |
| 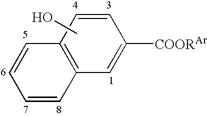 | |

| No. | HO-posit. | X¹ | X¹ | R^{Ar} | Suppl./Ref. |
|---|---|---|---|---|---|
| Q5-1-2-2-1 | 6- | — | — | -Me | TCI |
| Q5-1-2-2-2 | 6- | 5-Cl— | — | -Et | (ref1) |
| Q5-1-2-2-3 | 5-OH— | — | — | -Me | (ref2) |
| Q5-1-2-2-4 | 7-OH— | — | — | -Me | (ref3) |

(ref1) M. I. Dawson, et al., J. Med. Chem., 47, 3518 (2004)
(ref2) Kuo-Long Yu, et al., Bioorg. Med. Chem. Lett., 6, 2865 (1996)
(ref3) R. Gueller, et al., Bioorg. Med. Chem. Lett., 9, 1403 (1999)

TABLE 49

| | |
|---|---|
| | (Q5-1-3-1) |
| 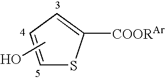 | |

| No. | HO-posit. | X¹ | X¹ | R^{Ar} | Suppl./Ref. |
|---|---|---|---|---|---|
| Q5-1-3-1-1 | 5- | — | — | -Et | (ref1) |
| Q5-1-3-1-2 | 4- | — | — | -Me | (ref2) |

(ref1) Jakobsen, et al., Tetrahedron, 19, 1867 ((1963)
(ref2) K. Kojima, et al., Bioorg. Med. Chem. Lett., 6, 1795 (1996)

TABLE 50

| | |
|---|---|
| | (Q5-1-4-1) |
| 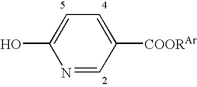 | |

| No. | X¹ | X¹ | R^{Ar} | Suppl./Ref. |
|---|---|---|---|---|
| Q-5-1-4-1-1 | — | — | -Me | (ref1) |
| Q-5-1-4-1-2 | 5-Cl— | — | -Me | (ref2) |
| Q-5-1-4-1-3 | 2-Me- | — | -Et | (ref3) |
| Q-5-1-4-1-4 | 5-Br— | — | -Me | (ref4) |
| Q-5-1-4-1-5 | 2-EtO— | — | -Et | (ref5) |
| Q-5-1-4-1-6 | 5-Me | — | -Me | (ref6) |
| Q-5-1-4-1-7 | 4-CF₃— | — | -Et | FluoroChem |

(ref1) A. Ling, et al., J. Med. Chem., 44, 3141 (2001)
(ref2) Graf, et al., J. Prakt. Chem., <2> 13, 244 (1933)
(ref3) Ramirez, et al., J. 0rg. Chem., 19, 183 (1954)
(ref4) v. Pechmann, et al., Chem. Ber., 37, 3837 (1904)
(ref5) Guthzeit, et al., J. Prakt. Chem, <2> 58, 425 (1898)
(ref6) Weglinski, et al., Rocz. Chem., 51 2401(1977)

TABLE 51

| | |
|---|---|
| | (Q5-1-4-2) |
| 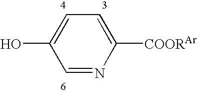 | |

| No. | X¹ | X¹ | R^{Ar} | Suppl./Ref. |
|---|---|---|---|---|
| Q5-1-4-2-1 | — | — | -Me | (ref1) |
| Q5-1-4-2-2 | 6-Br— | — | -Me | (ref2) |
| Q5-1-4-2-3 | 4-MeO— | — | -Me | (ref3) |

(ref1) Heyns, et al., Chem. Ber., 87, 13 (1954)
(ref2) T. R. Kelly, et al., J. Org. Chem., 61, 4623 (1996)
(ref3) W. C. Patt, et al., Tetrahedron Lett., 38, 1297 (1997)

TABLE 52

| | |
|---|---|
| | (Q5-1-6-1) |
| 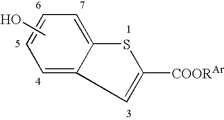 | |

| No. | HO-posit. | X¹ | X¹ | R^{Ar} | Suppl./Ref. |
|---|---|---|---|---|---|
| Q5-1-6-1-1 | 5- | — | — | -Me | (ref1) |
| Q5-1-6-1-2 | 5- | 3-Cl— | — | -Et | (ref2) |
| Q5-1-6-1-3 | 6- | 3-Me- | — | -Me | (ref3) |

(ref1) J. J. Lewis, et al., J. Med. Chem., 6, 711 (1963)
(ref2) M. Andrew, et al., Synthesis, 7, 1181 (1999)
(ref3) P. E.. Crass, et al., J. Med. Chem., 29, 1637 (1986)

TABLE 53

| | |
|---|---|
| | (Q5-1-6-2) |
| 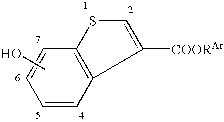 | |

| No. | HO-posit. | X¹ | X¹ | R^{Ar} | Suppl./Ref. |
|---|---|---|---|---|---|
| Q5-1-6-2-1 | 6- | — | — | -Me | (ref1) |
| Q5-1-6-2-2 | 7- | 6-Br— | — | -Et | (ref2) |
| Q5-1-6-2-3 | 5- | — | — | -Me | (ref1) |

(ref1) S. Mitsumori, et al, J. Med. Chem., 46, 2446 (2003)
(ref2) I. A. Kharizomenova, et al., Khim. Geterotsikl. Soedin., 12, 1626 (1984)

Furthermore, among the compounds represented by general formula (Q5-1), the compounds represented by general formulas (Q5-1-5-1-1), (Q5-1-5-2-1), (Q5-1-5-3-1), (Q5-1-7-1-1) and (Q5-1-7-2-1) can be easily produced according to the methods described in, for example, K. A. Parker, et al., Org. Lett., 4, 4265 (2002); E. Angeles, Molecules, 6, 683 (2001); V. Niementowski, et al., Chem. Ber., 49, 16 (1916); V. Georgian, et al., J. Org. Chem., 27, 4571 (1962); and Dieckmann, et al., Chem. Ber., 41, 3259 (1908), respectively.

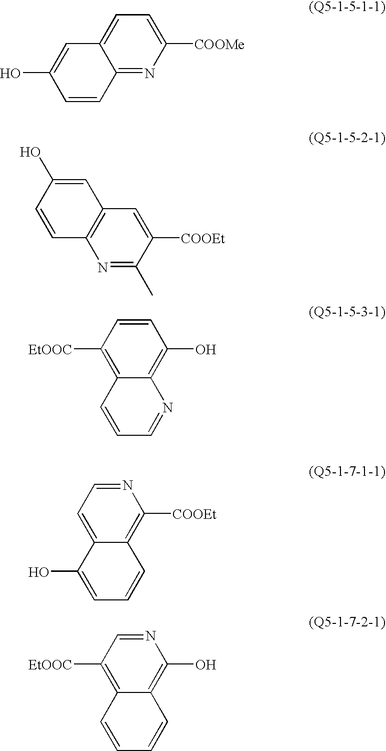

Also, a significant number of the compounds represented by general formulas (N2-3) and (N2-2) in the reaction scheme for Production method N1 are known, and the compounds may be commercially available or can be easily produced by an established known method, for example, the method shown in the reaction scheme for Production Method R1:

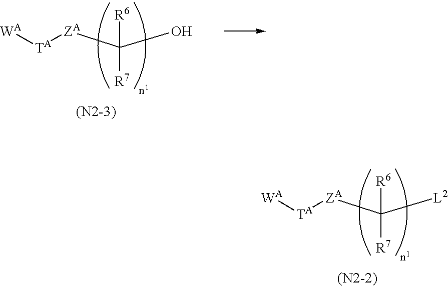

wherein WA, TA, Z^{A}, R⁶, R⁷ and L² have the same meanings as the defined above; and n¹ is 1 or 2.

The compound represented by general formula (N2-3) can be purchased from, for example, the companies described in (Table 54), or can be easily produced according to the literatures described in the footnotes of (Table 54).

TABLE 54

| | |
|---|---|
| | (N2-3) |
| 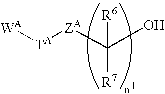 | |

| | | | | | | | Suppl./ |
|---|---|---|---|---|---|---|---|
| No. | W^{A} | T^{A} | Z^{A} | n¹ | R⁶ | R⁷ | Ref. |
| N2-3-1 | n-pentyl | —O— | -Ph- | 1 | — | — | Aldrich |
| N2-3-2 | F—(CH₂)₃— | —O— | -Ph- | 1 | — | — | Oakwood |
| N2-3-3 | n-butyl | — | -Ph- | 1 | — | — | Aldrich |
| N2-3-4 | i-pro- | — | -Ph- | 1 | — | — | TCI |
| N2-3-5 | t-butyl- | — | -Ph- | 2 | — | — | Lancaster |
| N2-3-6 | 2-Pyridyl- | — | thiophene | 1 | — | — | Maybridge |
| N2-3-7 | H— | — | thiophene | 2 | — | — | TCI |
| N2-3-8 | PhCH₂— | —O— | -(3-MeO)Ph- | 2 | — | — | (ref1) |

(ref1) Fujii, et al., Chem. Pharm. Bull., 35, 3628 (1987)

The compound represented by general formula (N2-2) can be purchased from, for example, the companies described in (Table 55).

TABLE 55

| | |
|---|---|
| | (N2-2) |
| 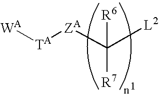 | |

| No. | W^{A} | T^{A} | Z^{A} | n¹ | R⁶ | R⁷ | L² | Suppl./Ref. |
|---|---|---|---|---|---|---|---|---|
| N2-2-1 | Ph | — | -Ph- | 1 | — | — | —Br | TCI |
| N2-2-2 | 4-F-Ph- | —O— | -Ph- | 1 | — | — | —Br | TCI |

Furthermore, the compound represented by general formula (N2-2) in which L² is an acyloxy group or a bromine atom can be produced from the compound represented by general formula (N2-3). For example, there may be listed a method which is the same method for producing the compound represented by general formula (2-7) from the compound represented by general formula (2-9).

The compound represented by the general formula (O2-11) used in the reaction scheme for Production Method O3 and the compound represented by the general formula (N2-32) used in the reaction scheme for Production Method N7 can be produced from the compound represented by general formula (N2-38), (N2-39) or (N2-40) shown below, and most of these compounds of general formula (N2-38), (N2-39) and (N2-40) are known, as in the compounds of general formulas (O2-11) and (N2-32). Thus, the compounds may be commercially available or can be produced by established known methods, for example, the methods shown in the reaction scheme for Production method R²:

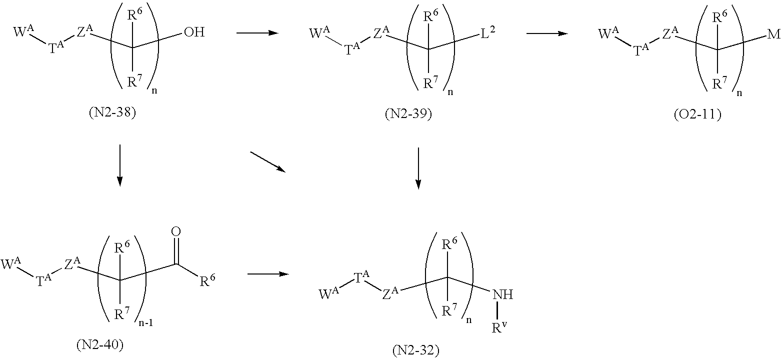

wherein WA, TA, Z^{A}, R⁶, R⁷, R^{V}, L², M and n have the same meanings as the defined above.

For example, a compound represented by general formula (N2-38) can be purchased from the companies described in (Table 56), or can be easily produced according to the literatures described in the footnotes of (Table 56).

TABLE 56

| | | | | | | | (N2-38) |
|---|---|---|---|---|---|---|---|
| 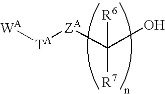 | | | | | | | |
| | | | | | | | Suppl./ |
| No. | W^{A} | T^{A} | Z^{A} | n¹ | R⁶ | R⁷ | Ref. |
| N2-38-1 | n-pentyl- | —O— | -Ph- | 1 | — | — | Aldrich |
| N2-38-2 | F—(CH₂)₃— | —O— | -Ph- | 1 | — | — | Oak- |
| | | | | | | | wood |
| N2-38-3 | n-butyl- | — | -Ph- | 1 | — | — | Aldrich |
| N2-38-4 | i-pro- | — | -Ph- | 1 | — | — | TCI |
| N2-38-5 | t-butyl- | — | -Ph- | 2 | — | — | Lan- |
| | | | | | | | caster |
| N2-38-6 | 2-Pyridyl- | — | thiophene | 1 | — | — | May- |
| | | | | | | | bridge |
| N2-38-7 | H— | — | thiophene | 2 | — | — | TCI |
| N2-38-8 | PhCH₂— | —NH— | -Ph- | 0 | — | — | TCI |
| N2-38-9 | PhCH₂— | —O— | -(3-MeO)Ph- | 2 | — | — | (ref1) |

(ref1) Fujii, et al., Chem. Pharm. Bull., 35, 3828 (1987)

A compound represented by general formula (N2-39) can be purchased from, for example, the companies described in (Table 57).

TABLE 57

| | |
|---|---|
| | (N2-39) |
| 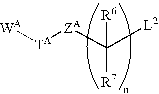 | |

| No. | W^{A} | T^{A} | Z^{A} | n¹ | R⁶ | R⁷ | L² | Suppl./Ref. |
|---|---|---|---|---|---|---|---|---|
| N2-39-1 | Ph | — | -Ph- | 1 | — | — | —Br | TCI |
| N2-39-2 | 4-F-Ph- | —O— | -Ph- | 1 | — | — | —Br | TCI |

Furthermore, a compound represented by general formula (N2-39) in which L² is an acyloxy group or a bromine atom, can be produced from the compound represented by general formula (N2-38). For example, there may be listed a method which is the same as the case of producing the compound represented by general formula (2-7) from the compound represented by general formula (2-9) as aforementioned with regard to the reaction scheme for Production Method D. The compound represented by general formula (O2-11) can be produced from the compound represented by general formula (N2-39). In this reaction, the metal atom or halogenated metal atom, M, may be exemplified by an alkali metal atom, or a halogenated alkaline earth metal atom. Li is preferable as the alkali metal atom, while MgCl, MgBr or MgI is preferable as the halogenated alkaline earth metal atom.

The metal reagent used in the reaction may be exemplified by alkyllithium such as tert-butyllithium, or magnesium.

For the solvent, amount of metal reagent, reaction temperature and reaction time used herein, for example, the conditions which are the same as those in the case of producing a compound represented by general formula (2-1) in which the terminal R² is a C1-4 alkyl group through a reaction with a metal reagent.

Furthermore, a compound represented by general formula (N2-32) can be purchased from, for example, the companies described in (Table 58), or can be produced, for example, by converting the compound represented by general formula (N2-38) to a compound represented by general formula (N2-40) through an oxidation reaction, and then subjecting the product to a reductive amination reaction. These two reactions can be performed on the basis of the method for obtaining the compound represented by general formula (2A) from the compound represented by general formula (2-1A) in which A is the group (2-1A-2), via an intermediate compound represented by general formula (2-1). Furthermore, there may also be listed another method as shown for the process for obtaining the compound represented by general formula (2-4) through a Mitsunobu reaction using the compound represented by general formula (2-9) and an imide compound and a deprotection reaction.

TABLE 58

| | |
|---|---|
| | (N2-32) |
| 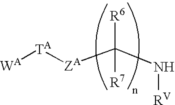 | |

| | | | | | | | | Suppl./ |
|---|---|---|---|---|---|---|---|---|
| No. | W^{A} | T^{A} | Z^{A} | n | R⁶ | R⁷ | R^{Y} | Ref. |
| N2-32-1 | CF₃— | — | -Ph- | 1 | — | — | —H | Aldrich |
| N2-32-2 | Ph- | — | -Ph- | 1 | — | — | —H | Aldrich |
| N2-32-3 | thiophene- | — | -thiophene- | 1 | — | — | —H | Maybridge |

The compound represented by general formula (N2-32) can also be produced from the compound represented by general formula (N2-39). For example, there may be listed a method which is the same as the case of producing the compound represented by general formula (2-4) from the compound represented by general formula (2-7).

Also, the compound represented by general formula (N2-40) can also be purchased from, for example, the companies described in (Table 59).

TABLE 59

| | | | | | | | | (N2-40) |
|---|---|---|---|---|---|---|---|---|
| 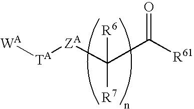 | | | | | | | | |
| | | | | | | | | Suppl./ |
| No. | W^{A} | T^{A} | Z^{A} | n¹ | R⁶ | R⁷ | R⁵¹ | Ref. |
| N2-40-1 | Me- | —O— | -Ph- | 1 | — | — | -Me | TCI |
| N2-40-2 | n-octyl- | —O— | -Ph- | 1 | — | — | -Me | Acros |
| N2-40-3 | Me- | — | -thiophene- | 1 | — | — | -Me | TCI |
| N2-40-4 | thio- | — | -thiophene- | 1 | — | — | —H | TCI |
| | phene- | | | | | | | |

A significant number of the compounds represented by general formulas (N2-28) and (O2-16) shown in the reaction schemes for Production methods N7 and O4, respectively, are known, and thus, the compounds may be commercially available or can be produced by established known methods, for example, the method shown in the reaction scheme for Production Method R3:

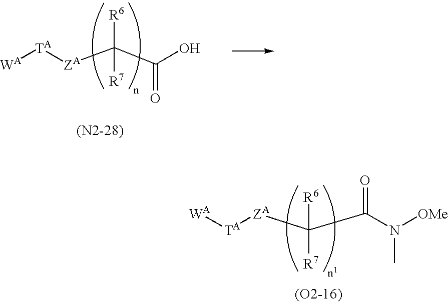

wherein W^{A}, T^{A}, Z^{A}, R⁶, R⁷ and n have the same meanings as the defined above.

The compound represented by general formula (N2-28) can be purchased from, for example, the companies described in (Table 60).

TABLE 60

| | |
|---|---|
| | (N2-28) |
| 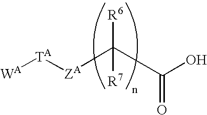 | |

| No. | W^{A} | T^{A} | Z^{A} | n | R⁶ | R⁷ | Suppl/Ref. |
|---|---|---|---|---|---|---|---|
| N2-28-1 | c-hexyl- | — | -Ph- | 0 | — | — | TCI |
| N2-28-2 | n-pentyl- | — | -Ph- | 0 | — | — | TCI |
| N2-28-3 | i-pro- | — | -Ph- | 0 | — | — | TCI |
| N2-28-4 | i-Pro- | — | -Ph- | 1 | — | — | Lancaster |

Also, for example, a significant number of the compounds represented by general formula (N2-28) can be produced by established known methods, for example, the method shown in the reaction scheme for Production Method R6.

For example, a compound represented by general formula (N2-28) in which TA is a single bond or a C1-C6 alkylene group, can be produced through a Suzuki reaction with the compounds shown in the above. For the Suzuki reaction, the method as described above may be listed.

Furthermore, a compound represented by general formula (N2-28) in which TA is —O—, can be produced by, for example, a method which is the same as the case of producing the compound represented by general formula (N2-5) through an alkylation reaction between the compound represented by general formula (N2-1) and the compound represented by general formula (N2-2). Moreover, a compound represented by general formula (N2-28) in which T is —S—, can be produced by, for example, a method which is the same as the case of producing the compound represented by general formula (N2-11) through a reaction of the compound represented by general formula (N2-4) and the compound represented by general formula (N2-10).

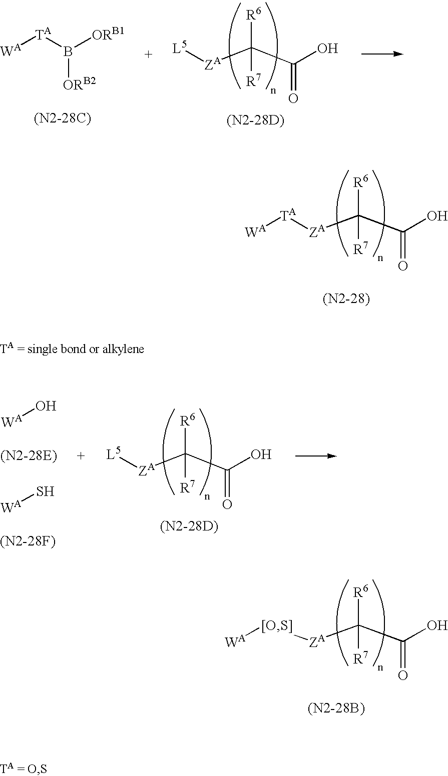

wherein W^{A}, T^{A}, R^{B1}, R^{B2}, L⁵, Z^{A}, R⁶, R⁷ and n have the same meanings as the defined above.

The compound represented by general formula (O2-16) can be produced through a condensation reaction between the compound represented by general formula (N2-28) and N,O-dimethylhydroxylamine hydrochloride. The reaction can be performed in the same manner as in the case of producing the compound represented by general formula (N2-30) from the compound represented by general formula (N2-28). The amount of N,O-dimethylhydroxylamine hydrochloride is preferably 0.5-fold or more the molar amount, and more preferably 1-fold the molar amount, of the compound represented by general formula (N2-28). Also, the amount is preferably 20-fold or less the molar amount, and more preferably 10-fold or less the molar amount, of the compound.

Furthermore, a significant number of the compounds represented by general formulas (N2-7), (N2-8), (N2-15), (N2-17) and (N2-26) respectively used in the reaction schemes for Production Methods N2, N4 and N6 are known, and the compounds may be commercially available, or can be produced by established known methods, for example, the methods shown in the reaction scheme for Production Method R⁴:

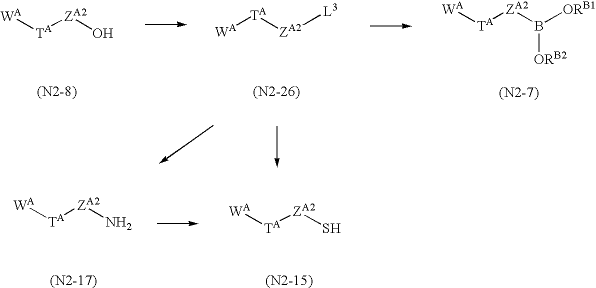

wherein W^{A}, T^{A}, Z^{A2}, L³, R^{B1} and R^{B2} have the same meanings as the defined above. The compound represented by general formula (N2-8) can be purchased from, for example, the companies described in (Table 61).

TABLE 61

| | |
|---|---|
| | (N2-8) |
| 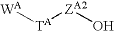 | |

| No. | W^{A} | T^{A} | Z^{A} | Suppl./Ref. |
|---|---|---|---|---|
| N2-8-1 | n-propyl- | —O— | -Ph- | TCI |
| N2-8-2 | n-pentyl- | —O— | -Ph- | TCI |
| N2-8-3 | tetrafluoroethyl- | —O— | -Ph- | WakO |
| N2-8-4 | n-butyl- | — | -Ph- | TCI |
| N2-8-5 | Ph- | —NH— | -Ph- | TCI |
| N2-8-6 | H— | — | -5-isoquinoline- | Aldrich |

The compound represented by general formula (N226) may be purchased from, for example, the companies described in (Table 62).

TABLE 62

| | |
|---|---|
| | (N2-26) |
| 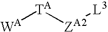 | |

| No. | W^{A} | T^{A} | Z^{A} | L³ | Suppl./Ref. |
|---|---|---|---|---|---|
| N2-26-1 | Me- | —O— | -Ph- | -OTf | Aldrich |
| N2-26-2 | Ph- | — | -thiophene- | -Br | Maybridge |
| N2-26-3 | H— | — | -5-isoquinoline- | -Br | Wako |

Furthermore, a compound represented by general formula (N2-26) in which L³ is an acyloxy group, can be produced from the compound represented by general formula (N2-8). For example, a method which is the same as the case of producing the compound represented by general formula (2-7) from the compound represented by general formula (2-9), may be listed.

In the case of the compound represented by general formula (N2-7), for example, the commercially available reagents described in (Table 63) may be used, or the compound can be produced through a reaction between the compound represented by general formula (N2-26) and a boron compound. For the reaction, there may be listed a method which is the same as the method for producing the compound represented by general formula (P2-2) by allowing the compound represented by general formula (O2-15) or the compound represented by general formula (O2-14) to react with a boron compound.

TABLE 63

| | |
|---|---|
| | (N2-7) |
| 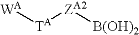 | |

| No. | W^{A} | T^{A} | Z^{A} | Suppl./Ref. |
|---|---|---|---|---|
| N2-7-1 | 2-Me- | — | -2-thiophene- | Aldrich |
| N2-7-2 | H— | — | -2-benzothiophene- | Aldrich |
| N2-7-3 | 2-thiophene- | — | -2-thiophene- | Maybridge |

The compound represented by general formula (N2-17) can be purchased from, for example, the companies described in (Table 64), or can be easily produced according to the literatures described in the footnotes of (Table 64).

TABLE 64

| | |
|---|---|
| | (N2-17) |
| 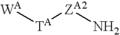 | |

| No. | W^{A} | T^{A} | Z^{A} | Suppl./Ref. |
|---|---|---|---|---|
| N2-17-1 | n-butyl- | — | -Ph- | TCI |
| N2-17-2 | Ph- | -4-SO₂— | -3-thiophene- | Maybridge |
| N2-17-3 | i-propyl- | — | -Ph- | TCI |
| N2-17-4 | Me- | — | -Ph- | (ref1) |

(ref1) Lee, et al., Org. Lett., 6, 1169(2005)

Furthermore, the compound represented by general formula (N2-17) can be produced by treating a solution of an organometallic compound obtained by metal-halogen exchange between the compound represented by general formula (N2-26) and an organolithium compound, with trimethylsilylmethylazide or the like. The organolithium compound used in the metal-halogen exchange reaction may be exemplified by n-butyllithium, sec-butyllithium, tert-butyllithium, or the like.

The type of the solvent used in the reaction is not particularly limited as long as the solvent is inactive to the reaction. Examples thereof include saturated hydrocarbon solvents, aromatic hydrocarbon solvents, or ether solvents. The saturated hydrocarbon solvent may be exemplified by pentane, hexane, heptane, cyclohexane or the like; the aromatic hydrocarbon solvent may be exemplified by toluene, xylene or the like; and the ether solvent may be exemplified by tetrahydrofuran, diethyl ether, 1,2-dimethoxyethane, 1,4-dioxane or the like. Pentane, hexane, cyclohexane, toluene, tetrahydrofuran, 1,2-dimethoxyethane, diethyl ether, or solvent mixtures of these solvents at any ratio are preferred.

The amount of the organometallic reagent used in the exchange reaction is preferably 0.1-fold or more the molar amount, and more preferably 0.5-fold or more the molar amount, of the compound represented by general formula (2-26). Also, the amount is preferably 10-fold or less the molar amount, and more preferably 5-fold or less the molar amount, of the compound.

The amount of trimethylsilylmethylazide or the like used in the reaction is preferably 0.5-fold or more the molar amount, and more preferably 1-fold or more the molar amount, of the compound represented by general formula (N2-26). Also, the amount is preferably 10-fold or less the molar amount, and more preferably 5-fold or less the molar amount, of the compound.

For the exchange reaction, the reaction temperature may vary depending on the raw material compound, solvent or the like, but typically, it is preferable to perform the reaction at −100° C. or higher, and it is preferable to perform the reaction at 0° C. or lower. The reaction time may vary depending on the raw material compound, solvent, reaction temperature or the like, but typically, the time may be, for example, from 5 minutes to 12 hours.

The compound represented by general formula (N2-15) can be purchased from, for example, the companies described in (Table 65).

TABLE 65

| | |
|---|---|
| | (N2-15) |
| 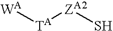 | |

| No. | W^{A} | T^{A} | Z^{A} | Suppl./Ref. |
|---|---|---|---|---|
| N2-15-1 | i-propyl- | — | -Ph- | TCI |
| N2-15-2 | H— | — | -thiophene- | TCI |

Furthermore, the compound represented by general formula (N2-15) can be produced, for example, by treating with sulfur, a solution of an organometallic compound obtained from the compound represented by general formula (N2-26) through metal-halogen exchange or magnesium addition. The organolithium compound used in the metal-halogen exchange reaction may be exemplified by n-butyllithium, sec-butyllithium, tert-butyllithium or the like. The type of the solvent used for the reaction is not particularly limited as long as the solvent is inactive to the reaction. Examples thereof include saturated hydrocarbon solvents, aromatic hydrocarbon solvents, or ether solvents. The saturated hydrocarbon solvent may be exemplified by pentane, hexane, heptane, cyclohexane or the like; the aromatic hydrocarbon solvent may be exemplified by toluene, xylene or the like; and the ether solvent may be exemplified by tetrahydrofuran, diethyl ether, 1,2-dimethoxyethane, 1,4-dioxane or the like. Pentane, hexane, cyclohexane, toluene, tetrahydrofuran, 1,2-dimethoxyethane, diethyl ether, or solvent mixtures of these solvents at any ratio are preferred.

The amount of the organometallic reagent used in the reaction is preferably 0.1-fold or more the molar amount, and more preferably 0.5-fold or more the molar amount, of the compound represented by general formula (N2-26). Also, the amount is preferably 10-fold or less the molar amount, and more preferably 5-fold or less the molar amount, of the compound.

For the reaction, the reaction temperature may vary depending on the raw material compound, solvent or the like, but typically, it is preferable to perform the reaction at −100° C. or higher, and it is preferable to perform the reaction at 0° C. or lower. The reaction time may vary depending on the raw material compound, solvent, reaction temperature or the like, but typically, the time may be, for example, from 5 minutes to 12 hours.

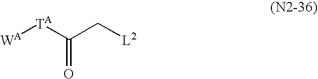

The compound represented by general formula (N2-36) in the reaction scheme for Production Method N8 in which TA is a single bond can be produced, for example, by halogenation of a compound having an acetyl group, such as acetophenone or 4-acetylpyridine (all manufactured by TCI, Inc.). Also, the same applies to the case where T^{A} is an alkylene group in the compound represented by general formula (N2-36). The halogenating agent used in the reaction may be exemplified by bromine, hydrobromic acid, or tetraalkylammonium tribromide such as tetrabutylammonium tribromide.

The solvent used in the reaction may be exemplified by aromatic hydrocarbon solvents, halogenated hydrocarbon solvents, ether solvents, acetic acid, or the like. The aromatic hydrocarbon solvent may be exemplified by toluene, xylene or the like; the halogenated hydrocarbon solvent may be exemplified by dichloromethane, chloroform or the like; and the ether solvent may be exemplified by tetrahydrofuran, diethyl ether, 1,4-dioxane or the like. Toluene, dichloromethane, chloroform, tetrahydrofuran, diethyl ether, acetic acid, or solvent mixtures of these solvents at any ratio are preferred.

The amount of the halogenating reagent used in the reaction is preferably an equimolar or more amount. For the reaction, the reaction temperature may vary depending on the raw material compound, solvent or the like, but typically, it is preferable to perform the reaction at 0° C. or higher, and more preferably at room temperature or above. The reaction time may vary depending on the raw material compound, solvent, reaction temperature or the like, but typically, the time may be from 5 minutes to 12 hours.

Meanwhile, the compound represented by general formula (N2-36) in which TA is —NR^{W}— can be produced, for example, through a condensation reaction between an amine compound such as 4-aminopyridine (manufactured by TCI, Inc.) and a halogenated acetic acid halide. The halogenated acetic acid halide used in the reaction may be exemplified by bromoacetic acid chloride, bromoacetic acid bromide, chloroacetic acid chloride, or the like. If necessary, the reaction may be performed in the co-presence of 1 to 10 equivalents, preferably 1 to 3 equivalents, of a base. The inactive solvent used in the reaction is not particularly limited as long as the solvent is inactive to the reaction. Examples thereof include ether solvents, halogenated hydrocarbon solvents, aromatic solvents, nitrile solvents, amide solvents, ketone solvents, sulfoxide solvents, or water. These may be used as mixtures of two or more kinds of them at appropriate ratios. Among them, acetonitrile, tetrahydrofuran, dichloromethane, chloroform and the like are preferred. For the base, there may be used a base which is the same as that used in performing the condensation reaction between the compound represented by general formula (N2-28) and the compound represented by general formula (N2-29) in the presence of a dehydrating-condensing agent. The base is preferably sodium hydride, potassium carbonate, sodium carbonate, sodium hydroxide, potassium hydroxide, sodium hydrogen carbonate, potassium hydrogen carbonate, triethylamine, pyridine or the like. The reaction temperature is usually preferably −20° C. or higher, and preferably 50° C. or lower. The temperature is more preferably ambient temperature. The reaction time may vary depending on the raw material compound, base, solvent, reaction temperature or the like, but typically, the time is preferably 5 minutes or more, and more preferably 1 hour or more. Also, the time is preferably 40 hours or less, and more preferably 18 hours or less.

Moreover, a significant number of the compounds represented by general formula (P2-1) as described in the reaction scheme for Production Method P1 are known, and the compounds can be purchased from, for example, the companies described in (Table 66), or can be easily produced according to the literatures described in the footnotes of (Table 66).

TABLE 66

| | |
|---|---|
| | (P2-1) |
| 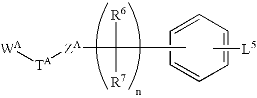 | |

| No. | W^{A} | T^{A} | Z^{A} | n | R⁶ | R⁷ | L⁵ | Suppl./Ref. |
|---|---|---|---|---|---|---|---|---|
| P2-1-1 | Ph- | —O— | — | 0 | — | — | -4-Br | TCI |
| P2-1-2 | 2-thiophene- | — | — | 0 | — | — | -4-Br | (ref1) |
| P2-1-3 | 4-Me-Ph- | —O— | — | 1 | — | — | -4-Br | (ref2) |

(ref1) Beadle, et al., J. Org. Chem, 49, 1594(1984)
(ref2) Huston, J. Am. Chem. Soc., 72, 4171(1950)

Likewise, with regard to the compound represented by general formula (P2-4) in the reaction scheme for Production Method PI, for example, the commercially available reagents described in (Table 67) can be used, or the compound can be easily produced according to the literatures described in the footnotes of (Table 67).

TABLE 67

| | |
|---|---|
| | (P2-4) |
| 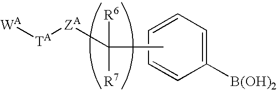 | |

| No. | W^{A} | T^{A} | Z^{A} | n | R⁶ | R⁷ | Suppl./Ref. |
|---|---|---|---|---|---|---|---|
| P2-4-1 | Ph- | —O— | — | 0 | — | — | Aldrich |
| P2-4-2 | 2-thiophene- | — | — | 0 | — | — | (ref1) |
| P2-4-3 | n-hexyl | — | — | 1 | — | — | (ref2) |

(ref1) Hotta, J. Heterocycl. Chem., 38, 923(2001)
(ref2) Marson, Tetrahedron., 59, 4377(2003)

Also, the compound represented by general formula (P2-4) can be produced, for example, as shown in the reaction scheme for Production Method R5:

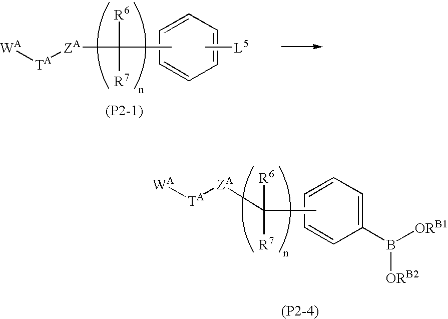

wherein W^{A}, T^{A}, Z^{A}, R⁶, R⁷, L⁵, R^{B1}, R^{B2} and n have the same meanings as the defined above,

by reacting the compound represented by general formula (P2-1) with a boron compound.

For the reaction, there may be listed a method which is the same as the method for producing the compound represented by general formula (P2-2) by allowing the compound represented by general formula (O2-15) or the compound represented by general formula (O2-14), to react with a boron compound.

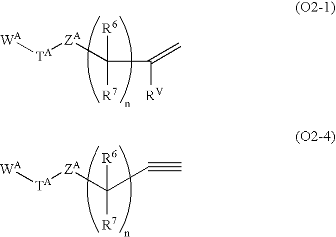

The compound represented by general formula (O2-1) is used in the reaction scheme for Production Method O1, and can be purchased from, for example, the companies described in (Table 68).

TABLE 68

| | |
|---|---|
| | (O2-1) |
| 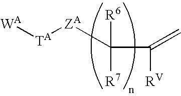 | |

| No. | W^{A} | T^{A} | Z^{A} | n | R⁶ | R⁷ | R⁹ | Suppl./Ref. |
|---|---|---|---|---|---|---|---|---|
| O2-1-1 | Ph- | — | -Ph- | 0 | — | — | —H | Aldrich |
| O2-1-2 | 4-CF₃— | — | -Ph- | 0 | — | — | —H | Aldrich |

Furthermore, the compound represented by general formula (O2-1) can be produced, for example, through a Wittig reaction or a Horner-Emmons reaction, between the compound represented by general formula (N2-40) and a phosphorus compound. The phosphorus compound may be exemplified by dimethyl methylphosphonate, methyltriphenylphosphonium bromide, or the like. For the conditions used in the reactions, the reactions can be performed on the basis of the known methods described in the aforementioned textbook (The Fourth Series of Lectures on Experimental Chemistry, Vol. 19, Maruzen) or the like. There are many other known methods described in the same book, and the compound can be produced on the basis of these methods.

The compound represented by general formula (O2-4) is used in the reaction scheme for Production Method O2, and the compound can be purchased from, for example, the companies described in (Table 69), or can be produced on the basis of the known methods described in the above-described textbook (The Fourth Series of Lectures on Experimental Chemistry, Vol. 19, Maruzen) or the like.

TABLE 69

| | |
|---|---|
| | (O2-4) |
| 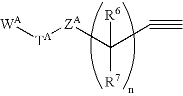 | |

| No. | W^{A} | T^{A} | Z^{A} | n | R⁶ | R⁷ | Suppl./Ref. |
|---|---|---|---|---|---|---|---|
| O2-4-1 | 4-Me- | — | -Ph- | 0 | — | — | Wako |
| O2-4-2 | 4-Ph- | — | -Ph- | 0 | — | — | Wako |
| O2-4-3 | 4-n-butyl- | — | -Ph- | 0 | — | — | Wako |
| O2-4-4 | 4-CF₃— | — | -Ph- | 0 | — | — | Aldrich |

In the reaction scheme for Production Method B, a significant number of the compounds represented by general formula (2-2) are known, and can be purchased from, for example, the companies described in (Table 70).

TABLE 70

| No. | Struct. | Suppl. |
|---|---|---|
| 2-2-1 | 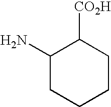 | Acros |
| 2-2-2 | 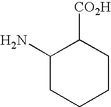 | Acros |
| 2-2-3 | 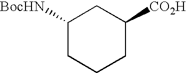 | Acros |
| 2-2-4 | 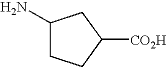 | Acros |
| 2-2-5 | 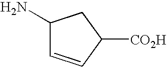 | Acros |
| 2-2-6 | 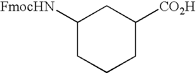 | Pep tech |
| 2-2-7 | 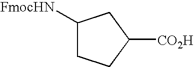 | Chem Impex |
| 2-2-8 | 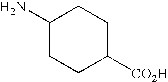 | TCI |
| 2-2-9 | 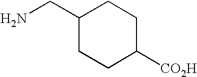 | TCI |
| 2-2-10 | 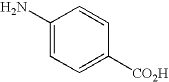 | TCI |
| 2-2-11 | 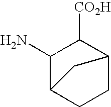 | Acros |
| 2-2-12 | 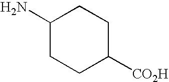 | TCI |
| 2-2-13 | 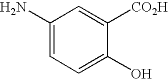 | KANTO |
| 2-2-14 | 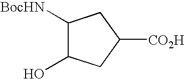 | Acros |
| 2-2-15 | 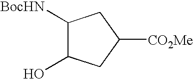 | Acros |
| 2-2-16 | 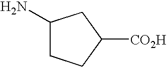 | Acros |
| 2-2-17 | 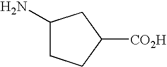 | Acros |
| 2-2-18 | 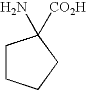 | TCI |
| 2-2-19 | 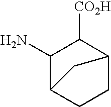 | Acros |
| 2-2-20 | 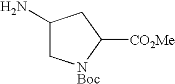 | Aldrich |
| 2-2-21 | 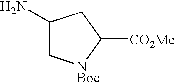 | Aldrich |
| 2-2-22 | 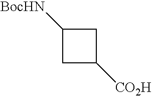 | AMRI |
| 2-2-23 | 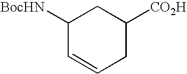 | AMRI |
| 2-2-24 | 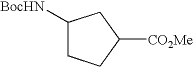 | Acros |
| 2-2-25 | 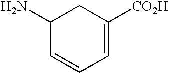 | Aldrich |
| 2-2-26 | 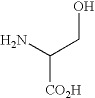 | WAKO |
| 2-2-27 | 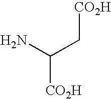 | SIGMA |
| 2-2-28 | 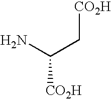 | SIGMA |
| 2-2-29 | 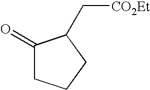 | Acros |
| 2-2-30 | 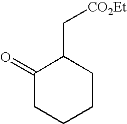 | Aldrich |
| 2-2-31 | 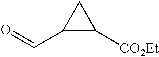 | Aldrich |

The methods for production of the compounds of the present invention are not limited to the methods described herein. For example, the compounds of the present invention can be produced by modifying or converting the substituents of compounds which serve as the precursors of the compounds of the invention, by means of one, or a combination of a plurality of the reactions described in conventional chemistry literatures and the like.

As an example of the production method for a compound containing asymmetric carbon among the compounds of the present invention, there may be used, in addition to the production method involving asymmetric reduction offered in the aforementioned, a method using a commercially available (or producible by a known method or a method equivalent to the known method) raw material compound, in which the moiety corresponding to asymmetric carbon has been rendered optically active in advance. Also, there is a method of separating optically active isomers from the compounds of the present invention or precursors thereof by conventional methods. For this method, there may be used, for example, a method of performing high performance liquid chromatography (HPLC) using an optically active column; a classical optical fractionation crystallization method including using an optically active reagent to form a salt, separating the salt using fractional crystallization, and then resolving the formation of salt; a method of separating and purifying diastereomers generated from the condensation of the compound with an optically active reagent, and then resolving the diastereomers again; or the like. When an optically active isomer is obtained by separating a precursor, the compound of the present invention which is optically active can be produced by performing the production methods described above thereafter.

Among the compounds of the present invention, if a compound contains an acidic functional group such as a carboxyl group, a phenolic hydroxyl group, or a tetrazole ring, the compound can also be formed into a pharmacologically acceptable salt (for example, an inorganic salt with sodium, ammonia or the like, or an organic salt with triethylamine or the like), by a known means. For example, when an inorganic salt is obtained, it is preferable to dissolve the compound of the present invention in water containing at least one equivalent of hydroxide, carbonate, bicarbonate or the like, which corresponds to the desired inorganic salt. For this reaction, a water-miscible inactive organic solvent, such as methanol, ethanol, acetone or dioxane, may be incorporated. For example, by using sodium hydroxide, sodium carbonate or sodium bicarbonate, a solution of sodium salt can be obtained.

Furthermore, among the compounds of the present invention, when a compound contains an amino group or contains other basic functional group in addition to the amino group, or when a compound contains an aromatic ring (for example, a pyridine ring, etc.) which itself has a basic nature, it is possible to convert the compound to a pharmacologically acceptable salt (for example, a salt with an inorganic acid such as hydrochloric acid or sulfuric acid, or a salt with an organic acid such as acetic acid or citric acid) by known means. For example, when an inorganic salt is obtained, it is preferable to dissolve the compound of the present invention in an aqueous solution containing at least one equivalent of the desired inorganic acid. For the reaction, a water-miscible inactive organic solvent, such as methanol, ethanol, acetone or dioxane, may be incorporated. For example, by using hydrochloric acid, a solution of a hydrochloride salt can be obtained.

Prodrugs of the compounds according to the present invention may include, but are not particularly limited to, compounds wherein a group constituting the prodrug is incorporated into at least one of any group selected from hydroxy group, amino group, and carboxyl group of the compound according to the present invention. In case of hydroxy group and amino group, a group constituting the prodrug may include an acyl group, and an alkoxycarbonyl group. Preferably, a group constituting the prodrug may include an acetyl group, a propionyl group, a methoxycarbonyl group, or an ethoxycarbonyl group, and an ethoxycarbonyl group is particularly preferable. Alternatively, an acetyl group is preferred in some embodiments. Alternatively, a propionyl group is preferred in some embodiments. Alternatively, a methoxycarbonyl group is preferred in some embodiments. In addition, in case of carboxyl group, a group constituting the prodrug may include a methyl group, an ethyl group, a n-propyl group, an isopropyl group, a n-butyl group, an isobutyl group, a s-butyl group, a t-butyl group, an amino group, a methylamino group, an ethylamino group, a dimethylamino group, or an diethyl amino group. Preferably the group may include an ethyl group, a n-propyl group, an isopropyl group, and an ethyl group is particularly preferred. Alternatively, a n-propyl group is particularly preferred in some embodiments. In some embodiments, an isopropyl group is preferred.

The compound according to the present invention may act as an immune-regulating drug useful in the treatment or prevention of autoimmune disease or chronic inflammatory disease since the compound has S1P1 agonist activity. The compound according to the present invention is useful, in the case that immunosuppression is at a normal condition, for example, in rejection of bone marrow, organ or graft and also in suppressing the immune system in autoimmune disease or chronic inflammatory disease, including systemic erythematodes, chronic rheumatic arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves' ophthalmopathy, or asthma

More particularly, the compound according to the present invention is useful in treating or preventing a disease or condition selected from the group consisting of transplantation of organ or tissue, graft versus host disease caused by transplantation, autoimmune syndrome (including rheumatic arthritis), systemic erythematodes, hashimotos thyroiditis, multiple sclerosis, myasthenia gravis, type I diabetes mellitus, uveitis, post uveitis, allergic encephalomyelitis, glomerulonephritis, postinfectious autoimmune disease (including rheumatic fever and postinfectious glomerulonephritis), inflammatory and hyperplastic skin disease, psoriasis, atopic dermatitis, contact dermatitis, eczematous dermatitis, seborrheic dermatitis, lichen planus, pemphigus, bullous pemphigus, epidermolysis bullosa, urticaria, angioedema, angiitis, erythema, skin eosinophilia, erythematodes, acne, alopecia areata, keratoconjunctivitis, spring conjunctivitis, uveitis involved in Behcet's disease, keratitis, herpetic kerato purulent inflammation, conical cornea, corneal epithelium degeneration, corneal leukoma, ocular pemphigus, Mooren's ulcer, scleratitis, Graves' Opthalmopathy, Vogt-Koyanagi-Harada syndrome, sarcoidosis, pollen allergy, reversible obstructive airway disease, bronchial asthma, allergic asthma, endogenous asthma, exogenous asthma, dust asthma, chronic or habitual asthma, late onset asthma and airway hyperreactive bronchiolitis, gastric ulcer, blood vessel injury caused by ischemic disease and thrombosis, ischemic bowel disease, inflammatory bowel disease, necrotizing enteritis, bowel disorder involved in burn, coeliac disease, rectitis, eosinophilic enterogastritis, adipocytichyperplasia, Crohn's disease, ulcerative colitis, migraine, rhinitis, eczema, interstitial nephritis, Good Pasture syndrome, hemolyticuremic syndrome, diabetic renal failure, multiple myositis, Gillian Bare syndrome, Meniere's syndrome, polyneuritis, multiple neuritis, mononeuritis, neuromuscular disorder, hyperthyroidism, Graves' disease, pure red cell aplasia, aplastic anemia, hypoplastic anemia, idiopathic thrombocytopenic purpura, autoimmune hemolytic anemia, agranulocytosis, pernicious anemia, megaloblastic anemia, erythropoiesis absence, osteoporosis, sarcoidosis, pulmonary fibrosis, idiopathic interstitial pneumonia, dermatomyositis, vitiligo vulgaris, ichthyosis vulgaris, photoallergic sensitivity, skin type T cell lymphoma, arteriosclerosis, atherosclerosis, aortitis syndrome, periarteritis nodosa, myocardiosis, pachyderma, Wegener's granulomatosis, Sjogren syndrome, adiposis, eosinophilic fasciitis, gingival disorder, periodontal disorder, alveolar bone disorder, cementum disorder, glomerulonephritis, andorogenetic alopecia or senile alopecia cause by preventing depilation or hair growth offer and/or the promotion of hair growth, muscular dystrophy, pustular dermatosis and Sezary syndrome, Addison disease, ischemic-reperfusion injury of organ caused by preservation or transplantation or ischemic disease, endotoxic shock, pseudomembranous colitis, colitis caused by drug or radiation, ischemic acute renal failure, chronic renal failure, toxicosis caused by lung-oxygen or drug, lung cancer, emphysema, cataract, siderosis, pigmentary retinitis, senile macular degeneration, hyaloid scarring, cornea alkali burn, multiforme exudativum erythema dermatitis, linear IgA bullous (ballous) dermatitis and cementum dermatitis, gingivitis, periodontitis, sepsis, pancreatitis, a disease caused by environmental pollution, a disease caused by aging, a disease caused by carcinogen, a disease caused by carcinoma metastasis, a disease caused by altitude sickness, a disease caused by release of histamine or leukotriene C4, Behcet's disease, autoimmune hepatitis, primary biliary cirrhosis, sclerosing cholangitis, partial hepatic excision, acute hepatic necrosis, toxic or viral hepatitis, or a necrosis caused by shock or anoxia, type B virus hepatitis, non-A/non-B type hepatitis, hepatic cirrhosis, alcoholic hepatic cirrhosis, hepatic failure, fulminant hepatic failure, late onset hepatic failure, “acute type chronic” hepatic failure, an enhancement of chemotherapy effect, cytomegalovirus infection, HCMV infection, AIDS, cancer, senile dementia, trauma, and chronic bacterial infection.

In addition, the present invention relates to a method of inhibiting or treating resistance for transplantation of organ or tissue or transplantation rejection in a mammal patient in need thereof, and also includes a method comprising administrating a therapeutically effective amount of the compound according to the present invention.

According to a still another example, a method of suppressing immune system in a mammal patient in need thereof, comprises administrating an immune system suppressing amount of the compound according to the present invention to the above patient.

Most particularly, as described in the present specification, a method of the treatment or prevention of bone marrow graft rejection or organ graft rejection may include a method comprising administering the compound according to the present invention or a pharmaceutically acceptable salt or hydrate thereof in an amount effective for the treatment or prevention of bone marrow graft rejection or organ graft rejection to a mammal patient in need thereof.

The compound according to the present invention is also useful in treating a respiratory disease or condition including asthma, chronic bronchitis, chronic obstructive pulmonary disease, adult respiratory distress syndrome, infant respiratory distress syndrome, cough, eosinophilic granuloma, respiratory syncytial virus bronchiolitis, bronchiectasis, idiopathic pulmonary fibrosis, acute pulmonary injury and obstructive bronchiolitis, organizing pneumonia

The compound according to the present invention including a salt and a hydrate thereof is useful in the treatment of autoimmune disease including the prevention of rejection of bone marrow graft, extrinsic organ graft and/or, pain, disease and disorder involved therein.

In addition, the compound according to the present invention is an S1P1 receptor selective agonist having selectivity for S1P1 receptor than S1P3 receptor. The S1P1 receptor selective agonist has a number of advantages than the current therapies, and thus, extends a therapeutic range of lymphocyte sequestering agent, so that the tolerance becomes higher as for higher compliance. Thus, S1P1 receptor selective agonist improves the efficacy as a single therapy.

The compound according to the present invention, a salt or a prodrug thereof has been researched to investigate selectivity for S1P1 receptor and S1P3 receptor, so that it is possible to represent a separation of an effect with undesired adverse reaction, bradycardia (reduction of heart rate) (The Journal of Pharmacology and Experimental Therapeutics, 309, 758-768, 2004).

In addition, the compound according to the present invention has a number of advantages than the current therapies by improving bradycardia, extends a therapeutic range of lymphocyte sequestering agent, so that the tolerance becomes higher as for higher compliance, and thus, improves the efficacy as a single therapy.

Furthermore, a pharmaceutical product comprising the compound according to the present invention as an active ingredient may be used in combination with at least one of another preventive or therapeutic agent for the aforementioned disease or disorder in a mammal, preferably a human, a pet or a companion animal such as a dog and a cat, or a domestic animal.

The agent capable of being used in combination with the compound according to the present invention includes: an immunosuppressant such as azathioprine, brequinar sodium, deoxyspergualin, mizaribine, mycophenolic acid morpholino ester, tacrolimus, cyclosporine, rapamycin and FTY720, and a formulation thereof; an immunomodulating antirheumatic agent used as a therapeutic agent for chronic rheumatic arthritis or antimetabolic agent, particularly a gold agent, such as bucilamine, lobenzarit, salazosulfapyridine, methotrexate, azathioprine, mizoribin, leflunomide, tacrolimus, cyclosporine, and a formulation thereof; a biological agent such as anticytokine antibody agents for cytokines such as interleukin (IL)-1, IL-6 or tumor necrosis factor (TNF)-α, or soluble receptor agents for the cytokines, particularly infliximab or ethanelsept, and a formulation thereof; a steroid agent such as dexamethasone, betamethasone, prednisolone, fluticasone or beclomethasone, and a formulation thereof; a bronchodilator used as a therapeutic agent for chronic bronchial asthma, particularly adrenaline β2 stimulant such as salmeterol or salbutamol, anticholinergic agent such as ipratropium, and a formulation thereof; a therapeutic agent for allergic disease, for example, xanthine analogues such as theophylline, antiallergic agents such as fexofenadine, epinastatin, cetirizine, ketotifen, sodium cromoglycate, pemirolast etc., or antihistaminic drugs such as fexoquinadine or cetirizine, and a formulation thereof; an anti-tumor agent such as irinotecan, 5-fluorouracil, and a formulation thereof. It is also illustrated to use a pharmaceutical product comprising the compound according to the present invention as active ingredient in combination with radiation therapy.

Each compound according to the present invention or a salt thereof, or a derivative thereof useful as a prodrug is excellent in safety (several toxicity or safety pharmacology) and pharmacokinetic property, and thus, an effectiveness of the compound as an active ingredient in a pharmaceutical product may be identified.

A test involved in safety includes, but is not limited to, cytotoxicity test (a test using HL60 cell or hepatocyte), genetic toxicity test (Ames test, mouse lymphoma TK test, chromosome abnormality test, micronucleus test), skin sensitization test (Buehler test, GPMT test, APT test, LLNA test), skin photosensitization test (Adjuvant and Strip method), eye stimulation test (once eye drop, short term continuous eye drop, repetitive eye drop), safety pharmacology test involved in cardiovascular system (telemetry method, APD method, hERG inhibition assay), safety pharmacology test involved in central nervous system (FOB method, Irwin's modified method), safety pharmacology test involved in respiratory system (a measuring method by the apparatus for measuring respiratory function, a measuring method by the apparatus for analyzing blood gas), general toxicity test.

Furthermore, a test for pharmacokinetic property includes, but is not limited to, a test for inhibiting or inducing cytochrome P450 enzyme, cell permeability test (a test using CaCO-2 cell or MDCK cell), drug transporter ATPase assay, oral absorption test, a test for measuring a concentration in blood, metabolic stability test (stability test, metabolism profiling test, reactive metabolism test), solubility test (solubility test according to turbidity)

It may be identified, for example, by a cell toxicity test that the compound according to the present invention or a salt thereof, or a derivative thereof useful as a prodrug is useful as an active ingredient in a pharmaceutical product. The cell toxicity test includes a method using several cultured cells, for example, a human pre-leukemia cell such as a HL-60 cell, a primarily cultured hepatocytes or a neutrophil fraction prepared from human peripheral blood. The present test may be practiced by, but not limited to, a method, in which cells are prepared as a cell suspension in a concentration of 10⁵ cell/ml to 10⁷ cell/ml, and 0.01 mL to 1 mL of the suspension is dispensed into micro tube or micro plate. A solution comprising the dissolved compound is added thereinto in an amount of 1/100 to 1 time of that of the cell suspension, and culture is conducted at 37° C. under 5% CO₂ for a period from 30 minutes to several days. After the completion of the culture, viability of the cells is evaluated by using MTT method or WST-1 method (Ishiyama, M., et al., In Vitro Toxicology, 8, p. 187, 1995). The effectiveness of the present compound as an active ingredient in a pharmaceutical product may be identified by measuring the cell toxicity of the present compound for the cells.

It may be identified, for example, by a genetic toxicity test that the compound according to the present invention or a salt thereof, or a derivative thereof useful as a prodrug is useful as an active ingredient in a pharmaceutical product. The genetic toxicity test includes Ames test, mouse lymphoma TK test, chromosome abnormality test, and micronucleus test. Ames test is a method of deciding mutation recovery by culturing bacterium on the culture dish incorporating the compound by using Salmonella or E. coli of the indicated bacteria species (Notification No. 1604 of the Evaluation and Licensing Division, PMSB, of 1999, “Genetic Toxicity Test Guideliner” II-1. Genetic Toxicity Test). In addition, mouse lymphoma TK test is a test for detecting a genetic mutagenesis using thymidine kinase gene of mouse lymphoma L5178Y cell as a target (Notification No. 1604 of the Evaluation and Licensing Division, PMSB, of 1999, “Genetic Toxicity Test Guideline” II-3. Mouse Lymphoma TK test; Clive, D. et al., Mutat. Res., 31, pp. 17-29, 1975; Cole, J., et al., Mutat. Res., 111, pp. 371-386, 1983). Furthermore, chromosome abnormality test is a method of detecting an activity causing a chromosome abnormality, comprising: culturing mammalian cells together with the compound, fixing the cells, staining the chromosomes, and observing the chromosomes (Notification No. 1604 of the Evaluation and Licensing Division, PMSB, of 1999, “Genetic Toxicity Test Guideline” II-2. Chromosome Abnormality Test using mammalian cultured cells). Still further, the micronucleus test may include a method of evaluating micronucleus formation ability derived from chromosome abnormality using rodents (in vivo test) (Notification No. 1604 of the Evaluation and Licensing Division, PMSB, of 1999, “Genetic Toxicity Test Guideline” II-4. Micronucleus Test using rodents; Hayashi, M. et al., Mutat. Res., 312, pp. 293-304, 1994; Hayashi, M. et al. Environ. Mol. Mutagen., 35, pp. 234-252, 2000) and using cultured cells (in vitro test) (Fenech, M. et al., Mutat. Res., 147, pp. 29-36, 1985; Miller, B., et al., Mutat. Res., 392, pp. 45-59, 1997). The effectiveness of the present compound as an active ingredient in a pharmaceutical product may be identified by defining genetic toxicity of the compound by using one or at least two of any of the aforementioned methods.

It may be identified, for example, by a skin sensitization test that the compound according to the present invention or a salt thereof, or a derivative thereof useful as a prodrug is useful as an active ingredient in a pharmaceutical product. The skin sensitization test includes Buehler method using a guinea pig as a skin sensitization test (Buehler, E. V. Arch. Dermatol., 91, pp. 171-177, 1965), GPMT method (maximization method (Magnusson, B. et al., J. Invest. Dermatol., 52, pp. 268-276, 1969)) or APT method (adjuvant & patch method (Sato, Y. et al., Contact Dermatitis, 7, pp. 225-237, 1981)). Still further, the skin sensitization test includes LLNA (Local Lymph node assay) method using a mouse as a skin sensitization test (OECD Guideline for the testing of chemicals 429, skin sensitization 2002; Takeyoshi, M. et al., Toxicol. Lett., 119 (3), pp. 203-8, 2001; Takeyoshi, M. et al., J. Appl. Toxicol., 25 (2), pp. 129-34, 2005). The effectiveness of the present compound as an active ingredient in a pharmaceutical product may be identified by defining skin sensitization of the compound by using one or at least two of any of the aforementioned methods.

It may be identified, for example, by a skin photosensitization test that the compound according to the present invention or a salt thereof, or a derivative thereof useful as a prodrug is useful as an active ingredient in a pharmaceutical product. The skin photosensitization test includes the skin photosensitization test using a guinea pig (“Drug Non-Clinical Test Guideline Explanation 2002” Yakuji Nippou Limited 2002, 1-9: Skin Photosensitization Test). The skin sensitization test includes Adjuvant and Strip method (Ichikawa, H. et al., J. Invest. Dermatol., 76, pp. 498-501, 1981), Harber method (Harber, L. C., Arch. Dermatol., 96, pp. 646-653, 1967), Horio method (Horio. T., J. Invest. Dermatol., 67, pp. 591-593, 1976), Jordan method (Jordan, W. P., Contact Dermatitis, 8, pp. 109-116. 1982), Kochever method (Kochever, I. E. et al., J. Invest. Dermatol., 73, pp. 144-146,1979), Maurer method (Maurer, T. et al, Br. J. Dermatol., 63, pp. 593-605, 1980), Morikawa method (Morikawa, F. et al., “Sunlight and man,” Tokyo Univ. Press, Tokyo, pp. 529-557, 1974), Vinson method (Vinson, L. J., J. Soc. Cosm. Chem., 17, pp. 123-130, 1966). The effectiveness of the present compound as an active ingredient in a pharmaceutical product may be identified by defining skin photosensitization of the compound by using one or at least two of any of the aforementioned methods.

It may be identified, for example, by an eye stimulation test that the compound according to the present invention or a salt thereof, or a derivative thereof useful as a prodrug is useful as an active ingredient in a pharmaceutical product. The eye stimulation test includes once eye drop test (dropping only once), short term continuous eye drop test (dropping for short term at several times at certain intervals) or repetitive eye drop test (dropping repetitively discontinuously for several days to several tens days), using a rabbit eye or a monkey eye. The eye stimulation test may include a method of evaluating symptoms of eye stimulation for a certain term after eye dropping according to an improved Draize score (Fukui, N. et al., Gendai no Rinsho, 4 (7), pp. 277-289, 1970). The effectiveness of the present compound as an active ingredient in a pharmaceutical product may be identified by defining eye stimulation of the compound by using one or at least two of any of the aforementioned methods.

It may be identified, for example, by a safety pharmacology test involved in cardiovascular system that the compound according to the present invention or a salt thereof, or a derivative thereof useful as a prodrug is useful as an active ingredient in a pharmaceutical product. The safety pharmacology test involved in cardiovascular system includes a telemetry method (a method of measuring the effect for electrocardiogram, heart rate, blood pressure, blood flow rates, under non-anesthesia by administering the compound (Sugano S., Tsubone H., Nakada Y., ed., electrocardiogram, echocardiography, blood pressure, pathological examination of animals for basis and clinic, 15, Maruzen, Co.)), an APD method (a method of measuring prolonged time of action potential of cardiomyocyte (Muraki, K. et al., AM. J. Physiol., 269, H524-532, 1995; Ducic, I. et al., J. Cardiovasc. Pharmacol., 30(1), pp. 42-54, 1997)), hERG inhibition assay (patch clamp method (Chachin, M. et al., Nippon Yakurigaku Zasshi, 119, pp. 345-351, 2002), Binding assay (Gilbert, J. D. et al., J. Pharm. Tox. Methods, 50, pp. 187-199, 2004), Rb+ efflex assay (Cheng, C. S. et al., Drug Develop. Indust. Pharm., 28, pp. 177-191, 2002), Membrane potential assay (Dorn, A. et al., J. Biomol. Screen, 10, pp. 339-347, 2005)). The effectiveness of the present compound as an active ingredient in a pharmaceutical product may be identified by defining the effect for cardiovascular system of the compound by using one or at least two of any of the aforementioned methods.

It may be identified, for example, by a safety pharmacology test involved in central nervous system that the compound according to the present invention or a salt thereof, or a derivative thereof useful as a prodrug is useful as an active ingredient in a pharmaceutical product. The safety pharmacology test involved in central nervous system includes FOB method (general evaluation method by observing functions (Mattson, J. L. et al., J. American College of Technology, 15 (3), pp. 239-254, 1996)), Irwin's modified method (a method of evaluating general symptoms and action observations (Irwin, S. Comprehensive Observational Assessment (Berl.) 13, pp. 222-257, 1968). The effectiveness of the present compound as an active ingredient in a pharmaceutical product may be identified by defining the effect for central nervous system of the compound by using one or at least two of any of the aforementioned methods.

It may be identified, for example, by a safety pharmacology test involved in respiratory system that the compound according to the present invention or a salt thereof, or a derivative thereof useful as a prodrug is useful as an active ingredient in a pharmaceutical product. The safety pharmacology test involved in respiratory system may include a method of measuring by measurement apparatus of respiratory functions (measuring respiration rate, tidal volume, minute ventilation) (Drorbaugh, J. E. et al., Pediatrics, 16, pp. 81-87, 1955; Epstein, M. A. et al., Respir. Physiol., 32, pp. 105-120, 1978) and a method of measuring by analysis apparatus of blood gas (measuring blood gas, or hemoglobin oxygen saturation degree) (Matsuo, S. Medicina, 40, pp. 188-, 2003). The effectiveness of the present compound as an active ingredient in a pharmaceutical product may be identified by defining the effect for respiratory system of the compound by using one or at least two of any of the aforementioned methods.

It may be identified, for example, by a general toxicity test that the compound according to the present invention or a salt thereof, or a derivative thereof useful as a prodrug is useful as an active ingredient in a pharmaceutical product. The general toxicity test may include a method of observations of general conditions, clinical chemical changes or pathological tissue changes of the administered animals by administering orally or intravenously, the compound dissolved or suspended in an appropriate solvent once or repetitively (over several days) using any rodents such as rats or mice, or non-rodents such as monkey or dog. The effectiveness of the present compound as an active ingredient in a pharmaceutical product may be identified by defining the general toxicity of the compound by using one or at least two of any of the aforementioned methods.

It may be identified, for example, by a test for inhibiting or inducing cytochrome P450 enzyme (Gomez-Lechon., M. J. et al., Curr. Drug Metab. 5(5), pp. 443-462, 2004) that the compound according to the present invention or a salt thereof, or a derivative thereof useful as a prodrug is useful as an active ingredient in a pharmaceutical product. The test may include a method of measuring whether the compound inhibits the activity of the enzyme in-vitro using cytochrome P450 enzyme of each molecular species or human P450 expression microsome purified from cells or prepared by using recombinant gene expression system (Miller, V, P. et al., Ann. N.Y. Acad. Scil., 919, pp, 26-32, 2000), a method of measuring the changes of the expression of cytochrome P450 enzyme of each molecular species or the activity of the enzyme using human hepatic microsome and cellular fragment solution (Hengstler, J. G. et al., Drug Metab. Rev., 32, pp. 81-118, 2000), or a method of investigating an enzyme induction ability of the compound by extracting RNA from human hepatic cells exposed to the compound, and comparing mRNA expression mass to the control (Kato, M. et al., Drug Metab. Pharmacokinet., 20(4), pp. 236-243, 2005). The effectiveness of the present compound as an active ingredient in a pharmaceutical product may be identified by defining the effect for enzyme inhibition or enzyme induction of cytochrome P450 enzyme of the compound by using one or at least two of any of the aforementioned methods.

It may be identified, for example, by a cell permeability test that the compound according to the present invention or a salt thereof, or a derivative thereof useful as a prodrug is useful as an active ingredient in a pharmaceutical product. The cell permeability test includes a method of measuring a cell membrane permeability of the compound in cell culture medium in vitro using CaCO-2 cell (Delie, F. et al., Crit. Rev. Ther. Drug Carrier Syst., 14, pp. 221-286, 1997; Yamashita, S. et al., Eur. J. P ham. Sci., 101 pp. 195-204, 2000; Ingels, F. M. et al., J. Pham. Sci., 92, pp. 1545-1558, 2003), or a method of measuring a cell membrane permeability in cell culture medium in vitro using MDCK cell (Irvine, J. D. et al., J. Pham. Sci., 88, pp. 28-33, 1999). The effectiveness of the present compound as an active ingredient in a pharmaceutical product may be identified by defining the cell permeability of the compound by using one or at least two of any of the aforementioned methods.

It may be identified by performing, for example, a drug transporter ATPase assay that a compound according to the present invention, a salt thereof, or a derivative thereof useful as a prodrug is useful as an active ingredient of a drug. As the drug transporter ATPase assay, there is a method of determining whether or not the compound is a substrate of P-glycoprotein (P-gp) by using a P-gp baculovirus expression system (Germann, U. A., Methods Enzymol., 292, pp. 427-41, 1998). The effectiveness as an active ingredient for a pharmaceutical product may be identified by defining an effect of the compound to the P-gp using the method.

It may be identified by performing, for example, an oral absorbability test that a compound according to the present invention, a salt thereof, or a derivative thereof useful as a prodrug is useful as an active ingredient of a drug. As an example of the oral absorbability test, there is a method of dissolving or suspending a predetermined amount of a compound in a suitable solvent, measuring time-varying blood drug concentration of the compound that is oral-administered into a rodent, a monkey, or a dog, and evaluating an blood drug transportability associated with the oral administration of the compound by using an LC-MS/MS method (Newest Mass Spectrometry for Bioscience, edited by HARADA Kenichi et al, Kodansha Scientific, 2002). The effectiveness as an active ingredient for a pharmaceutical product may be identified by defining oral absorbability of the compound using the method.

It may be identified by performing, for example, a blood concentration transition measuring test that a compound according to the present invention, a salt thereof, or a derivative thereof useful as a prodrug is useful as an active ingredient of a drug. As an example of the blood concentration transition measuring test, there is a method of measuring a transition of blood concentration of a compound that is administered into a rodent, a monkey, or a dog by using an LC-MS/MS method (Newest Mass Spectrometry for Bioscience, edited by HARADA Kenichi et al, Kodansha Scientific, 2002). The effectiveness as an active ingredient for a pharmaceutical product may be identified by defining a blood concentration transition of the compound using the method.

It may be identified by performing, for example, a metabolism test that a compound according to the present invention, a salt thereof, or a derivative thereof useful as a prodrug is useful as an active ingredient of a drug. As an example of the metabolism test, there are aplasmastability test (a method of estimating in-vivo metabolism clearance from a metabolism speed of a compound in a liver microsome of a human or other animals (Shou, W. Z. et al., J, Mass Spectrom., 40(10), pp. 1347-1356, 2005; Li, C. et al., Drug Meta b. Dispos., 34 (6), 901-905, 2006)), a metabolism profiling test, and a reactive metabolism test. The effectiveness as an active ingredient for a pharmaceutical product may be identified by defining a metabolism profile of the compound using one or two or more of the aforementioned methods.

It may be identified by performing, for example, a solubility test that a compound according to the present invention, a salt thereof, or a derivative thereof useful as a prodrug is useful as an active ingredient of a drug. As an example of the solubility test, there is a solubility test using a turbidity method (Lipinski, C. A. et al., Adv. Drug Deliv. Rev., 23, pp. 3-26, 1997; Bevan, C. D. et a 1., Anal. Chem., 72, pp. 1781-1787, 2000). The effectiveness as an active ingredient for a pharmaceutical product may be identified by defining solubility of the compound using the method.

As a pharmaceutical product according to the present invention, a compound according to the present invention, one of pharmacologically acceptable salts thereof, or a mixture of two or more thereof may be used. Preferably, a pharmaceutical product composition which is obtained by adding one or more of pharmacologically acceptable carriers to one or more of compounds according to the present invention or pharmacologically acceptable salts thereof may be administered. The pharmacologically acceptable carrier is not limited to a specific one. Examples thereof include an excipient, a binding agent, a destructive agent, a lubricant, and an additive. The excipient is, for example, D-mannitol. The binding agent is, for example, carboxtymethyl cellulose. The destructive agent is, for example, cornstarch. The lubricant is, for example, glycerin. The additive is, for example, para-oxybenzoic acid ester. In addition, the additive is, for example, a surfactant such as polyoxyethylenesorbitan monooleate (Tween80) or HC-60.

In administration of the pharmaceutical product according to the present invention to a human body, the pharmaceutical product may be orally administered in a type of a tablet, a powder, a granule, a capsule, a sugar-coated pill, a solution, or a syrup. In addition, the pharmaceutical product may be non-orally administered in a type of an injection product, a drip, a suppository, a transepidermal agent, or an absorptive agent. In addition, the pharmaceutical product may be absorbed in an air spray such as aerosol or dry powder.

A dosing period of the pharmaceutical product according to the present invention is not specifically limited. In case of therapy, the dosing period may be basically selected as a period that clinical symptom of each disease is determined to appear. Generally, the dosing period may be several weeks or one year. However, according to the pathological condition, the dosing period may be prolonged. In addition, although the clinical symptom disappears, the administration may be continued. Furthermore, although the clinical symptom does not appear, the administration may be performed in terms of disease prevention according to the discretion of doctor. A dosage of pharmaceutical product according to the present invention is not specifically limited. In general, for an adult, one-day suitable amount, that is, 0.01 to 2000 mg of an active ingredient may be administered one time, or its divided amount may be administered several times. As a dosing frequency, everyday or monthly administration may be available. Preferably, one time/week to three times/week, or five time/week, or everyday administration may be performed. The one-day dosing amount, the dosing period, and the dosing frequency need to be suitably increased or decreased according to age, weight, physical healthiness, and medical history or a disease severity of a patient.

The pharmaceutical product according to the present invention may be used in combination with a preventive pharmaceutical product or a treatment pharmaceutical product for other disorder and disease.

### EXAMPLE

Hereinafter, examples of the present invention are described in detail, but the scope of the present invention is not limited to the following examples.

In the following examples, various analyses are performed as follows

### (1) Fast Atom Bombardment Mass Spectrometry (FAB-MS)

JMS-AX500 mass spectrometer or JMS-SXI02 mass spectrometer manufactured by JEOL Ltd was used for measurement. 3-nitro benzyl alcohol was used as a matrix.

### (2) Liquid Chromatograph Mass Spectrometry (LC-MS)

As a mass spectrometer, Platform-LC mass spectrometer (using an electro-spray ionization (ESI) method) manufactured by Micromass Ltd in England was used. As a liquid chromatography apparatus, an apparatus manufactured by GILSON Ltd. in France was used. As a separation column, Mightysil RP-18 GP 50-4.6 RP -18 GP 50-4.6 (Product No. 25468-96) manufactured by Kanto Chemical Co., Inc (Japan) was used. The elution condition is listed below.

Flow Rate: 2 mL/min

### Solvent:

Solution A=water, 0.1% (v/v) acetic acid contained
Solution B=acetnitril, 0.1% (v/v) acetic acid contained

For the time interval of 0 min to 5 min, Solution B is represented with 5 to 100% (v/v) straight line gradient. The elution time was shown in minute.

### (3) Nuclear Magnetic Resonance Spectrometry (NMR)

The measurement is performed by using Gemini-300 (FT-NMR, manufactured by Varian). As a solvent, dichloroform (CDCl₃), dimethanol (CD₃OD), or dimethyl sulfoxide (DMSO-d⁶) was used, and if not particularly limited, CDCl₃ was used for the measurement. Chemical shift was represented by δ (ppm) using tetra methylsilane (TMS) as an internal standard, and a binding constant was represented by J (Hz). In addition, as splitting pattern symbols, a singlet was represented by s; a doublet was represented by d; a triplet was represented by t; a quartet was represented by q; a quintet was represented by qu; a doublet doublet was represented by dd; a triplet doublet was represented by td; a multiplet was represented by m; and a broad was represented by b.

### (4) Thin Layer Chromatography (TLC)

TLC plate (silica gel 60F₂₅₄, Product No. 1,05715) manufactured by Merck in Germany was used. After development, the TLC plate was illuminated with UV beams having a wavelength of 254 nm, and a compound detection was performed by using a general detection method.

### (5) Refining Chromatography

Basically, any one of the following methods was used. (Refining Method 1) Flash Column System (manufactured by biotage) was used. One or several cartridge columns of KP-Sil-12M, 40S or 40M manufactured by Biotage were used according to an amount of sample

(Refining Method 2) A general column chromatography was conducted, and silica gel 60N (spherical shape, neutral, 40 to 100 μm, manufactured by Kanto Chemical Co., Inc) was used according to an amount of sample

Hereinafter, in the examples, “LCMS” denotes a liquid chromatograph mass Spectrometry, and “RTime” denotes a sustaining time of a liquid chromatograph. Mass Spectrometry data of the LCMS is indicated by “Mass” in Tables and “ESI-MS” except for the Tables. “FAB-MS” denotes a mass Spectrometry data measured by using a fast atom bombardment mass spectrometry.

Meanings of symbols in Table are as follows. “Exp.” denotes an example compound number; “Ref.” denotes a reference example number; “Syn.” denotes a manufacturing method; “SM” denotes a raw-material compound; “Reagent” denotes a site where the SM is acquired; and “Structure” denotes the structure of an example compound. In addition, symbols written in “Reagent” of Table denote a manufacturer of samples. “TCI” denotes a product manufactured by Tokyo Chemical Industry Co., Ltd; “KAN” denotes a product manufactured by Kanto Chemical Co., Inc; “Ald” denotes a product manufactured by Aldrich Chemical Company; “KOK” denotes a product manufactured by manufactured by Kokusan Kagaku Company; “WAKO” denotes a product manufactured by Wako Pure Chemical Industries, Ltd.; “LAN” denotes a product manufactured by Landcaster Company; “ACR” denotes a product manufactured by Acros Chemical Company; “SIG” denotes a product manufactured by Sigma Chemical Company; “AMRI” denotes a product manufactured by AMRI; “Pep” denotes a product manufactured by Peptech; and “Che” denotes a product manufactured by Chemlmpex. In addition, “Syn” denotes a synthetic product.

### Reference Example 1

### (1R,4s)-4-aminocyclopentane-2-encarboxylic acid hydrochloric acid salt

To (1R,4s)-4-(tert-butoxycarbonyl)aminocyclopentane-2-encarboxylic acid (50 mg, manufactured by Fluka), 2 mL of 4N HCl dioxane solution was added. The resultant mixture was stirred for 2 hours at room temperature, and a solvent was distilled away under a reduced pressure. As a result, the titled compound was obtained.

### Example 1

### (1R,4s)-4-(4-((4-phenyl-5-(trifluoromethyl) thiophene-2-yl)methoxy)benzylamino)cyclopentan-2-encarboxylic acid

To a dichloromethane solution (4 mL) of (1R,4s)-4-aminocyclopentane-2-encarboxylic acid hydrochloric acid salt (50 mg) obtained in Reference Example 1 and 4-((4-phenyl-5-(trifluoromethyl)thiophen-2-yl)methoxy)benzaldehyde (72 mg, Synthetic Product 1), a triacetoxy sodium borohydride (83.9 mg, manufactured by Aldrich) was added. The resultant mixture was stirred for 6 hours at room temperature. After the stirring was ended, a reaction mixture was concentrated under a reduced pressure.

Next, a chromatography using a Biotage 12M cartridge (as an elution solution, 10:1:0.1 (v/v) of dichloromethane/methanol/25% ammonia aqueous solution was used) was performed, so that 30.5 mg of the titled compound was obtained. ESI-MS: 474 (M+H), RTime 4.09 min.

The aforementioned “Synthetic Product 1” compound was synthesized based on a method disclosed in the document of J. Med. Chem. vol. 47, p. 6222 (2004). Hereinafter, the same is applied.

### Examples 2 to 29

According to the procedures of Example 1, the same method was performed except that any one of raw compounds as listed in Table 71 was used instead of (1R,4s)-4-aminocyclopentan-2-encarboxylic acid hydrochloride. As a result, the compounds of Examples 2 to 29 listed in Table 71 were obtained.

TABLE 71

| | | | | | LCMS | |
|---|---|---|---|---|---|---|
| Exp | Syn. | SM | Suppl. | Struct. | Rtime | Mass |
| 2 | A | 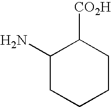 | ACR | 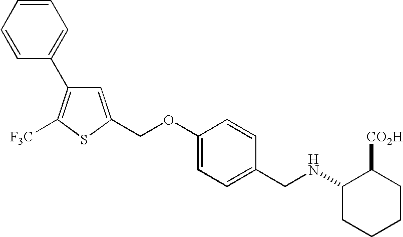 | 4.23 | 490 |
| 3 | A | 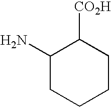 | ACR | 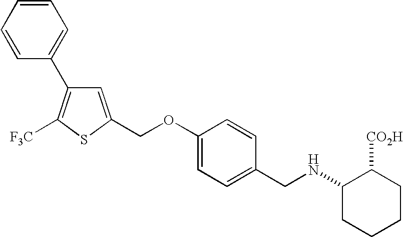 | 4.22 | 490 |
| 4 | A | 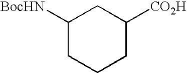 | ACR | 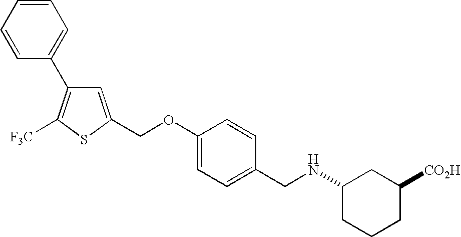 | 3.89 | 490 |
| 5 | A | 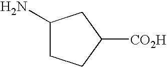 | ACR | 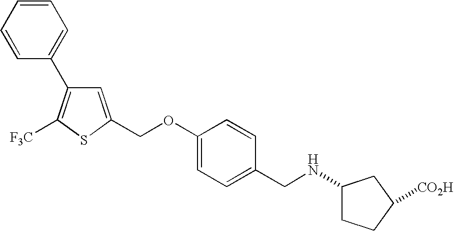 | 3.87 | 476 |
| 6 | A | 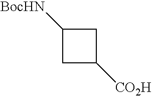 | AMRI | 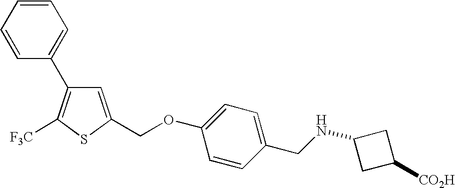 | 3.82 | 462 |
| 7 | A | 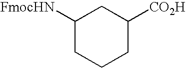 | Pep | 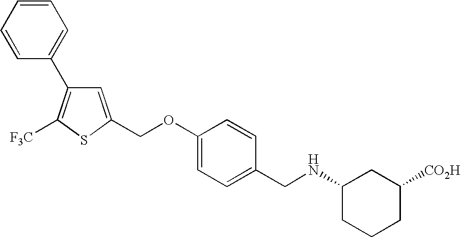 | 3.89 | 490 |
| 8 | A | 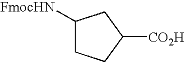 | Che | 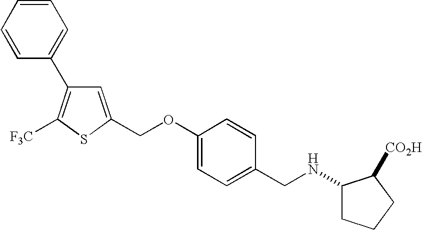 | 4.09 | 476 |
| 9 | A | 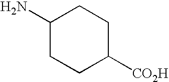 | TCI | 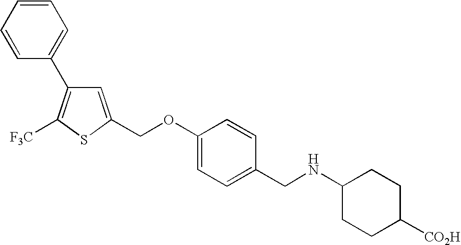 | 3.90 | 490 |
| 10 | A | 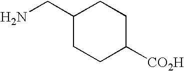 | TCI | 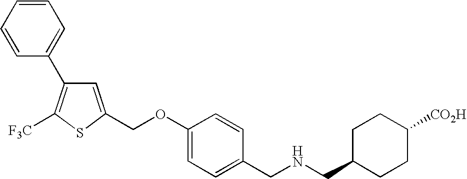 | 3.91 | 504 |
| 11 | A | 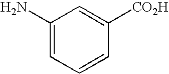 | TCI | 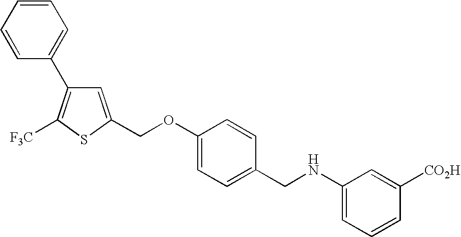 | 5.57 | 484 |
| 12 | A | 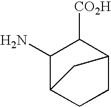 | ACR | 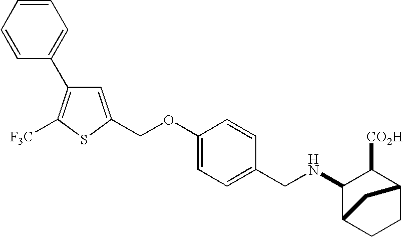 | 4.34 | 502 |
| 13 | A | 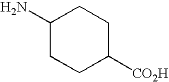 | TCI | 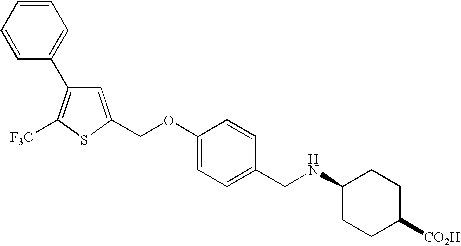 | 3.88 | 490 |
| 14 | A | 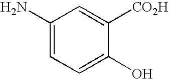 | KAN | 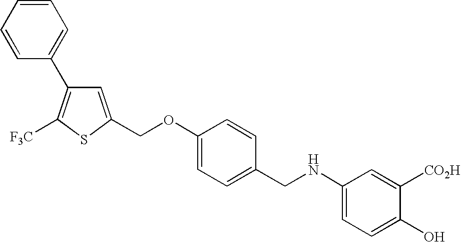 | 5.31 | 500 |
| 15 | A | 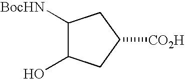 | ACR | 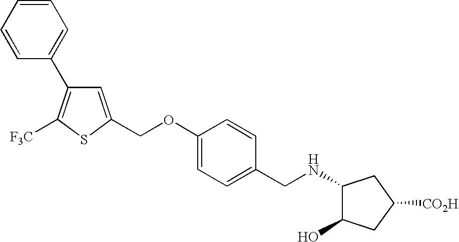 | 3.82 | 492 |
| 16 | A | 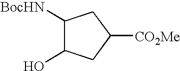 | ACR | 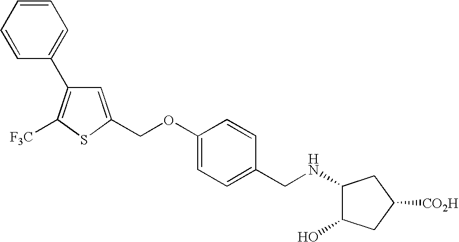 | 3.90 | 492 |
| 17 | A | 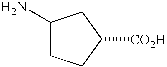 | ACR | 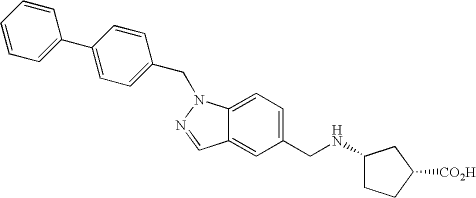 | 3.59 | 426 |
| 18 | A | 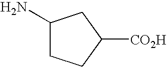 | ACR | 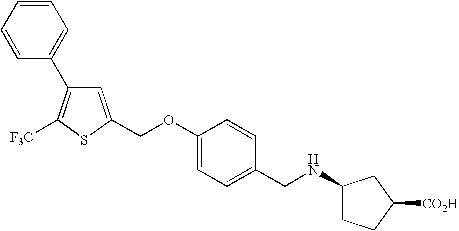 | 3.91 | 476 |
| 19 | A | 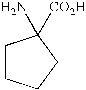 | TCI | 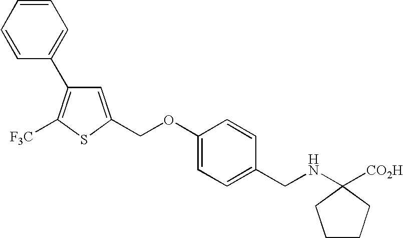 | 4.20 | 476 |
| 20 | A | 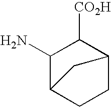 | ACR | 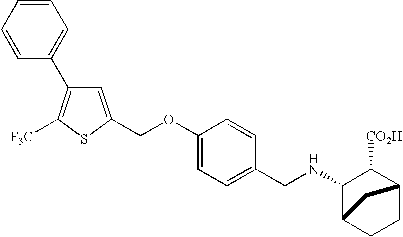 | 4.34 | 502 |
| 21 | A | 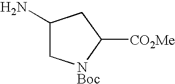 | Ald | 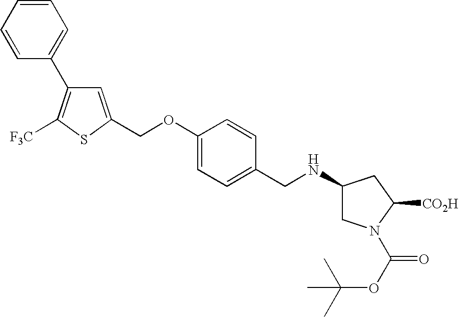 | 4.37 | 577 |
| 22 | A | 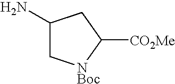 | Ald | 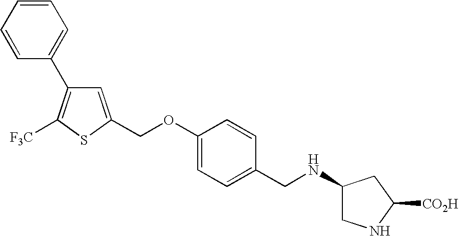 | 3.58 | 477 |
| 23 | A | 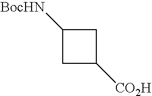 | AMRI | 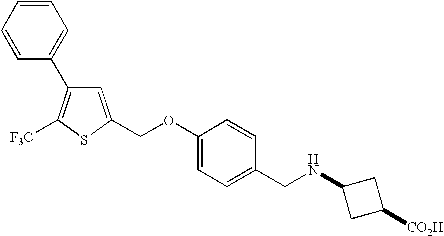 | 3.79 | 432 |
| 24 | A | 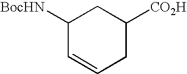 | AMRI | 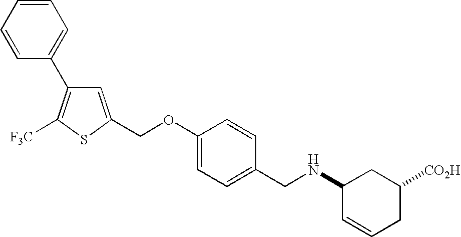 | 3.82 | 488 |
| 25 | A | 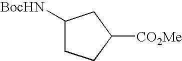 | ACR | 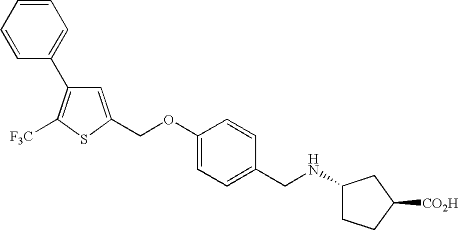 | 3.78 | 476 |
| 26 | A | 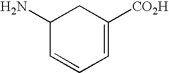 | Ald | 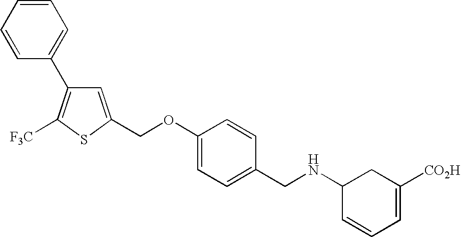 | 4.31 | 486 |
| 27 | A | 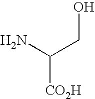 | WAKO | 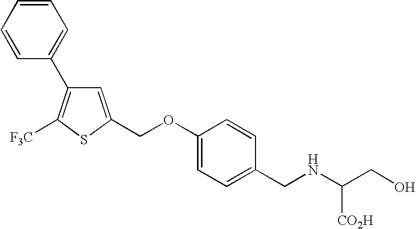 | 4.92 | 452 |
| 28 | A | 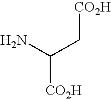 | SIG | 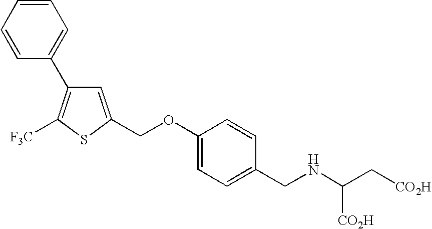 | 4.17 | 480 |
| 29 | A | 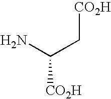 | SIG | 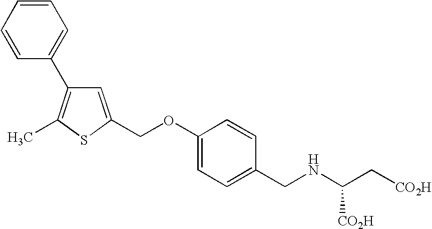 | 4.20 | 480 |

### Reference Example 2

### (4-((4-phenyl-5-(trifluoromethyl) thiophen-2-yl)methoxy)phenyl)methanol

To a methanol solution (5 mL) of 4-((4-phenyl-5-(trifluoromethyl)thiophen-2-yl)methoxy)benzaldehyde (198.6 mg, Synthetic Product 1), a sodium borohydride (20.7 mg) was added. The resultant mixture was stirred for 20 minutes at 0° C. After the stirring was ended, a saturated sodium bicarbonate aqueous solution was added to the reaction solution, and extraction was performed by using ethyl acetate. An organic layer was washed with a saturated saline solution, dried by using magnesium sulfate, and concentrated under a reduced pressure. As a result, 224.3 mg of the titled compound was obtained. The obtained residue was directly used for the next reaction.

¹H-NMR (CDCl₃): 7.89-7.69 (9H, m), 7.42 (1H, s) 5.19 (1H, s), 4.80 (1H, s), 3.01 (2H, s).

### Reference Example 3

### 2-(4-((4-phenyl-5-(trifluoromethyl) thiophen-2-yl)methoxy)benzyl)isoindoline-1,3-dione

To a dehydrated tetrahydrofuran solution (20 mL) of (4-((4-phenyl-5-(trifluoromethyl)thiophen-2-yl)methoxy)phenyl)methanol (608.5 mg) obtained in Reference Example 2, futalimide (737.1 mg, manufactured by Kanto Chemical Co., Inc), tri-normal butylphosphine (625.6 μL, manufactured by Kanto Chemical Co., Inc), and 1,1′-azobis(N,N N,N′-dimethyl formamide) (432.2 mg, manufactured by Midori Kagaku Co., Ltd.) were added under a nice-cooled condition. The resultant mixture was stirred for 2 hours at room temperature under a nitrogen ambience. After the stirring was ended, the filtrate was concentrated under a reduced pressure. A saturated sodium bicarbonate aqueous solution was added to the residue, extraction was preformed by using ethylacetate. An organic layer was washed with a saturated saline solution, dried by using magnesium sulfate, and a solvent was distilled away under a reduced pressure. Subsequently, achromatography (as an elution solution, 3:1 (v/v) of hexane/ethyl acetate was used) using Biotage 40s cartridge was performed, so that 1.02 g of the titled compound was obtained. ESI-MS: 494 (M+H).

### Reference Example 4

### (4-((4-phenyl-5-(trifluoromethyl) thiophen-2-yl)methoxy)phenyl)methane amine

2-(4-((4-Phenyl-5-(trifluoromethyl)thiophen-2-yl)methoxy)benzyl)isoindoline-1,3-dione (1.02 g) obtained in Reference Example 3 was dissolved in ethanol (20 mL) and hydradin monohydrate (160 μL, manufactured by Tokyo Chemical Industry Co., Ltd.) was added to the solution, and the resultant mixture was stirred for 72 hours at room temperature. After the stirring was ended, a generated product was removed by using a filter, and the filtered solution was concentrated under a reduced pressure. A saturated sodium bicarbonate solution was added to the residue, and extraction was performed by using chloroform. An organic layer was dried by using anhydrous sodium sulfate, and a solvent was distilled away under a reduced pressure. Subsequently, achromatography (as an elution solution, 9:1 (v/v) of chloroform/methanol was used) using Biotage 40s cartridge was performed, so that 629.2 mg of the titled compound was obtained. ESI-MS: 364 (M+H), RTime 3.72 min.

### Example 30

### (E)-4-(4-((phenyl-5-(trifluoromethyl)thiophen-2-yl)methoxy)benzylamino)-2-butenoic acid methyl ester

To a dichloromethane solution (3 mL) of (4-((4-phenyl-5-(trifluoromethyl)thiophen-2-yl)methoxy)phenyl)methane amine (51.3 mg) obtained in Reference Example 4 and fumaric aldehydemethylester (15.9 mg, manufactured by Tokyo Chemical Industry Co., Ltd.), acetic acid (10 μL), and sodium triacetoxyborohydride (59.3 mg, manufactured by Aldrich) were added. The resultant mixture was stirred for 72 hours at room temperature. After the stirring was ended, a reaction mixture was concentrated under a reduced pressure. Subsequently, a chromatography (as an elution solution, 9:1 (v/v) of chloroform/methanol was used) using Biotage 12M cartridge was performed, so that 25.9 mg of the titled compound was obtained. ESI-MS: 462 (M+H).

### Example 31

### (E)-4-(4-((phenyl-5-(trifluoromethyl)thiophen-2-yl)methoxy)benzylamino)-2-butenoic acid

To a tetrahydrofuran solution (10 mL) of (E)-4-(4-((phenyl-5-(trifluoromethyl)thiophen-2-yl)methoxy)benzylamino)-2 butenoic acid methyl (87.6 mg) obtained in Example 30, a lithium hydroxide aqueous solution (10 wt %, 1 mL) was added. The resultant mixture was stirred for 72 hours at 45° C. After the stirring was ended, the reaction mixture was concentrated at a reduced pressure. Subsequently, a chromatography (as an elution solution, 40:10:1 (v/v/v) of dichloromethane/methanol/25% ammonia aqueous solution was used) using Biotage 12M cartridge was performed, so that 57.6 mg of the titled compound was obtained. ESI-MS: 448 (M+H), RTime 3.83 min.

### Example 32 to 34

In the flowing examples listed in Table 72, according to the procedures of Example 30, the same method was performed except that any one of raw compounds listed in Table 72 was used instead of fumaric aldehyde methyl ester. As a result, the compounds of the following examples were obtained.

TABLE 72

LCMS

| Exp | Syn. | SM | Suppl. | Struct. | Rtime | Mass |
|---|---|---|---|---|---|---|
| 32 | A | 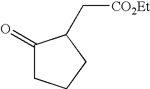 | ACR | 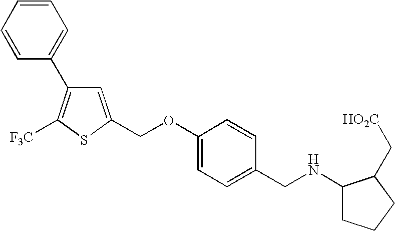 | 4.03 | 490 |
| 33 | A | 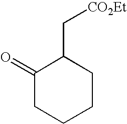 | Ald | 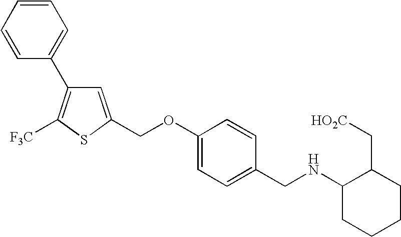 | 4.23 | 504 |
| 34 | A | 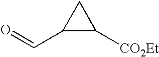 | Ald | 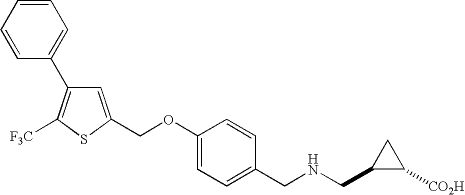 | 3.81 | 462 |

### Reference Example 5

### (1H-indazol-6-yl)methanol

To a tetrahydrofuran solution (1.14 L) of methyl indazole-6-carboxylate (20.0 g) that was synthesized based on a method disclosed in the document of J, Med. Chem. vol. 43, p. 41 (2000), lithium aluminum hydride (8.62 g, manufactured by Wako Pure Chemical Industries, Ltd.) was added at 0° C. The resultant mixture was stirred for 2.5 hours at room temperature. After the stirring was ended, 2N sodium hydroxide aqueous solution (114 mL, manufactured by Wako Pure Chemical Industries, Ltd.) was added to the mixture at 0° C., and the resultant blend was filtered by using Celite. The filtered solution was dried, and a solvent was distilled away under a reduced pressure. Subsequently, chloroform was added to the residue and the resultant mixture was filtered. Drying was performed, so that 12.8 g of the titled compound was obtained. ESI-MS: 149 (M+H), RTime 3.01 min.

### Reference Example 6

### 1H-indazole-6-carboaldehyde

To an ethyl acetate solution (861 mL) of (1H-indazol-6-yl)methanol (8.61 g) obtained in Reference Example 5, IBX (17.1 g, Synthetic Product 2) was added. The resultant mixture was stirred for 17 hours at 90° C. After the stirring was ended, filtering was performed, and the filtered solution was distilled away under a reduced pressure. Subsequently, a column chromatography (as an elution solution, 100:0 to 96:4 (v/v) of chloroform/methanol was used) using silica gel 60 N was performed, so that 7.69 g of the titled compound was obtained. ESI-MS: 147 (M+H), RTime 3.34 min.

“Synthetic Product 2” was synthesized based on a method disclosed in the document of J. Org. Chem. vol. 64, p. 4537 (1999).

### Reference Example 7

### 1-(4-phenylbenzyl)-1H-indazole-6-carboaldehyde

To an N,N-dimethyl formamide solution (3.3 mL) of 1H-indazole-6-carboaldehyde (48.4 mg) obtained in Reference Example 6, potassium carbonate (91.2 mg) and 4-bromomethylbiphenyl (106 mg, manufactured by Tokyo Chemical Industry Co., Ltd.) were added. The resultant mixture was stirred for 22 hours at 50° C. After the stirring was ended, a saturated ammonium chloride aqueous solution (10 mL) was poured into the reaction mixture solution, and extraction was performed by using ethyl acetate (20 mL×2). An organic layer was washed with a saturated saline solution and dried, and a solvent was distilled away under a reduced pressure.

Subsequently, a chromatography (as an elution solution, 3:1 (v/v) of hexane/ethyl acetate was used) using Biotage 12M cartridge was performed, so that 49.2 mg of the titled compound was obtained.

¹H-NMR (CDCl₃) 5.71 (2H, s), 7.28-7.45 (5H, in), 7.49-7.56 (4H, m), 7.68 (1H, d, J=8.4), 7.87 (1H, d, J=8.4), 7.93 (1H, s), 8, 15 (1H, s), 10.13 (1H, s).

### Example 35

### (1R,3s)-3-N-({1-(4-phenylbenzyl)-1H-indazol-6-yl)methyl}aminocyclopentane carboxylic acid

To a 1,2-dichloroethane solution (1.6 mL) of 1-(4-phenylbenzyl-)-1H-indazole-6-carboaldehyde (49.2 mg) obtained in Reference Example 7, acetic acid (37.9 L) and sodium triacetoxyborohydride (67.0 mg, manufactured by Aldrich) were added. The resultant mixture was stirred for 14 hours at room temperature. After the stirring was ended, a saline solution (3 mL) was added to the reaction mixture solution, and extraction was performed by using dichloromethane (10 mL×2). An organic layer was washed with a saturated saline solution and dried, and a solvent was distilled away under a reduced pressure. Subsequently, achromatography (as an elution solution, 100:10:1 (v/v/v) of dichloromethane/methanol/25% ammonia aqueous solution was used) using Biotage 12M cartridge was performed, so that 35.3 mg of the titled compound was obtained. ESI-MS: 426 (M+H), RTime 3.62 main.

### Example 36

In the following examples listed in Table 73, according to the procedures of Example 35, the same method was performed except that 2H-indazole-6-carboaldehyde was used instead of 1-(4-phenylbenzyl)-1H-indazole-6-carboaldehyde. As a result, the compound of Example 36 was obtained.

TABLE 73

| | | | | | LCMS | |
|---|---|---|---|---|---|---|
| Exp | Syn. | SM | Suppl. | Structure | Rtime | Mass |
| 36 | A | 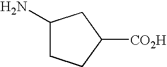 | ACR | 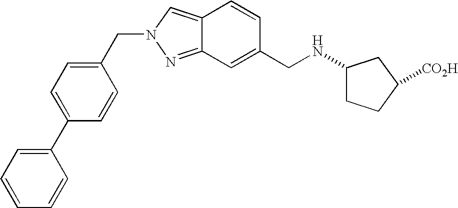 | 3.59 | 426 |

### Reference Example 8

### (1H-indazol-5-yl)methanol

To a tetrahydrofuran solution (1.00 L) of methyl indazole-5-carboxylate methyl ester (6.91 g) that was synthesized based on the method disclosed in the document of J. Med. Chem. vol. 43, p. 41 (2000), lithium aluminum hydride (2.98 g, manufactured by Wako Pure Chemical Industries, Ltd.) was added at 0° C. The resultant mixture was stirred for 2.5 hours at room temperature. After the stirring was ended, 2N sodium hydroxide aqueous solution (39.22 mL, manufactured by Wako Pure Chemical Industries, Ltd.) was added to the mixture at 0° C., and the resultant blend was filtered by using Celite. The filtrate was dried, and the solvent was distilled away under a reduced pressure. Subsequently, chloroform was added to the residue and the resultant mixture was filtered. Drying was performed, so that 4.12 g of the titled compound was obtained.

### Reference Example 9

### 1H-indazole-5-carboaldehyde

To an ethyl acetate solution (600 mL) of (1H-indazol-5-yl)methanol (6.00 g) obtained in Reference Example 8, IBX (11.9 g, Synthetic Product 2) was added. The resultant mixture was stirred for 17 hours at 90° C. After the stirring was ended, the mixture was filtered, and the filtered solution was distilled away under a reduced pressure. Subsequently, a column chromatography (as an elution solution, 100:0 to 94:6 (v/v) of chloroform/methanol was used) using silica gel 60N was performed, so that 5.74 g of the titled compound was obtained.

“Synthetic Product 2” was synthesized based on a method disclosed in the document of J. Org. Chem. vol. 64, p. 4537 (1999).

### Reference Example 10

### 1-benzyl-1H-indazole-5-carboaldehyde

To an N,N-dimethyl formamide solution (3.0 mL) of 1H-indazole-5-carboaldehyde (200 mg) obtained in Reference Example 9, potassium carbonate (379.0 mg) and benzylbromide (325 μL, manufactured by Wako Pure Chemical Industries, Ltd.) were added. The resultant mixture was stirred for 5 hours at room temperature. After the stirring was ended, a saturated ammonium chloride aqueous solution (10 mL) was poured into the reaction mixture solution, and extraction was performed by using ethyl acetate (20 mL×2). An organic layer was washed with a saline solution and dried, and the solvent was distilled away under a reduced pressure. Subsequently, chromatography (as an elution solution, 3:1 (v/v) of hexane/ethyl acetate was used) using Biotage 12M cartridge was performed, so that 204.9 mg of the titled compound was obtained. ESI-MS: 237 (M+H), RTime 4.48 min.

### Reference Example 11

### (1-benzyl-1H-indazol-5-yl)methanol

To a methanol solution (2 mL) of 1-benzyl-1H-indazole-5-carboaldehyde (204.9 mg) obtained in Reference Example 10, polystyrene supporting-borohydride (200 mg, manufactured by NovabioChem) was added, and the resultant mixture was stirred for 2 hours at room temperature. After the stirring was ended, the reaction solution was filtered. The solvent was distilled away, so that 191.1 mg of the titled compound was obtained. ESI-MS: 239 (M+H), RTime 4.00 min.

### Reference Example 12

### 3-(4-acetoxybenzylamino) propanoic acid t-butyl ester

To a dichloromethane solution (30 mL) of 4-acetoxy benzaldehyde (667 μL, manufactured by Tokyo Chemical Industry Co., Ltd.) and β-alanine t-butyl ester hydrochloride (905.4 mg, manufactured by SIGMA), sodium triacetoxyborohydride (1.21 g, manufactured by Aldrich) was added. The resultant mixture was stirred for 12 hours at room temperature. After the stirring was ended, the reaction solution was poured into a saturated saline solution, extraction was performed by using dichloromethane, drying was performed, and a solvent was distilled away under a reduced pressure.

¹H-NMR (CDCl₃): 7.34-7.24 (2H, m), 7.05 (2H, d, J=19.5), 4.16 (2H, s), 3.55-3.83 (2H, m), 2.51-2.24 (2H, m), 2.10 (3H, s), 1.43 (9H, s).

### Reference Example 13

### 3-(4-acetoxybenzyl-t-butoxycarbonylamino)propanoic acid t-butyl ester

To a dichloromethane solution (50 mL) of 3-(4-acetoxybenzylamino)propanoic acid t-butyl ester obtained in Reference Example 12, triethylamine (759 μL, manufactured by Wako Pure Chemical Industries, Ltd.) and di-t-butylcarbonate (1.14 g, manufactured by Tokyo Chemical Industry Co., Ltd.) were added. The resultant mixture was stirred for 2 hours at room temperature. After the stirring was ended, the reaction solution was poured into water, and extraction was performed by using ethyl acetate. An organic layer was washed with a saturated saline solution and dried, and the solvent was distilled away under a reduced pressure. Subsequently, chromatography (as an elution solution, 7:1 (v/v) of hexane/ethyl acetate was used) using Biotage 40s cartridge was performed, so that 1.23 g of the titled compound was obtained.

¹H-NMR (CDCl₃): 7.35-7.23 (2H, m), 7.05 (2H, d, J=19.5), 4.16 (2H, s), 3.55-3.83 (2H, m), 2.51-2.24 (2H, m), 2.10 (3H, s), 1.43 (18H, s).

### Reference Example 14

### 3-(4-hydroxybenzyl-t-butoxycarbonylamyl)propanoic acid t-butyl ester

To a methanol solution (30 mL) of 3-(4-acetoxybenzyl-t-butoxycarbonylamino)propanoic acid t-butyl ester (1.22 g) obtained in Reference Example 13, potassium carbonate (471 mg, manufactured by Kokusan Kagaku Company) was added. The resultant mixture was stirred for 30 minutes at room temperature. After the stirring was ended, the reaction solution was filtered. The filtered solution was concentrated under a reduced pressure, so that 1.08 g of the titled compound was obtained.

¹H-NMR (CDCl₃): 7.26 (2H, m), 6.77 (2H, d, J=8.7), 5.07 (1H, s), 4.36 (2H, s), 3.34 (2H, m), 2.42 (2H, m), 1.43 (18H, s)

### Reference Example 15

### 3-(4-(1-benzyl-1H-indazole-5-yl)methoxy)benzyl-t-butoxy carbonylaminopropanoic acid t-butyl ester

To a tetrahydrofuran solution (2 mL) of 3-(4-hydroxy benzyl-t-butoxycarbonylamino)propanoic acid t-butyl ester (35.0 mg) obtained in Reference Example 14 and (1-benzyl-1H-indazol-5-yl)methanol (47.7 mg) obtained in Reference Example 11, tri-normal butylphosphine (50.0 μL, manufactured by Kanto Chemical Co., Inc) and 1,1′-azobis (N,N′-dimethyl formamide) (34.4 mg, manufactured by Midori Kagaku Co., Ltd.) were added. The resultant mixture was stirred for 12 hours at room temperature under a nitrogen ambience. After the stirring was ended, the product was removed by using a filter, and the filtered solution was concentrated under a reduced pressure Subsequently, chromatography (as an elution solution, 5:1 (v/v) of hexane/ethyl acetate was used) using Biotage 12M cartridge was performed, so that 50.4 mg of the titled compound was obtained.

¹H-NMR (CDCl₃): 8.04 (1H, s), 7.92 (1H, s), 7.79-7.18 (10H, m), 5.60 (2H, s), 5.12 (2H, s), 4.37 (2H, s), 3.49-3.44 (2H, m), 2.44 (2H, m), 1.55 (18H, s).

### Example 37

### 3-(4-(1-benzyl-1H-indazol-5-yl)methoxy)benzylaminopropanoic acid

To 3-(4-(1-benzyl-1H-indazol-5-yl)methoxy)benzyl-t-butoxycarbonylaminopropanoic acid t-butyl ester (50.4 mg) obtained in Reference Example 15, 4N HCl dioxane solution (2 mL) was added. The resultant mixture was stirred for 24 hours at room temperature. After the stirring was ended, the solvent was distilled away. As a result, 50.0 mg of the titled compound was obtained.

¹H-NMR (DMSO-d⁶): 9.18 (1H, s)), 8.12 (1H, s), 7.85 (1H, s), 7.72 (1H, d, J=8.7), 7.45 (3H, d, J=8.7), 7.33-7.22 (5H, m), 7.07 (2H, d, J=8.7), 5.64 (2H, s), 5.18 (2H, s), 4.05 (2H, s), 3.04 (2H, t, J=5.7), 2.71 (2H, t, J=5.7)

### Reference Example 16

### 3-(4-cyanobenzylamino)propanoic acid t-butyl ester

To a dichloromethane solution (200 mL) of 4-cyanobenzaldehyde (9.10 g, manufactured by TCI) and β-alanine t-butyl ester hydrochloride (13.25 q, manufactured by Kokusan Kagaku Company), sodium triacetoxyborohydride (17.6 g, manufactured by Aldrich) was added. The resultant mixture was stirred for 4 hours at room temperature. After the stirring was ended, a saturated sodium bicarbonate aqueous solution was added to the reaction solution, and extraction was performed by using dichloromethane. An organic layer was washed with a saturated saline solution, dried by using magnesium sulfate, and concentrated under a reduced pressure, so that the titled compound was obtained. The obtained residue was directly used for the next reaction.

### Reference Example 17

### 3-(4-cyanobenzyl-t-butoxycarbonylamino)propanoic acid t-butyl ester

To a dichloromethane solution (250 mL) of 3-(4-cyanobenzylamino)propanoic acid t-butyl ester obtained in Reference Example 16, triethylamine (14.2 mL, manufactured by Wako Pure Chemical Industries, Ltd.) and di-t-butyl carbonate (18.2 g, manufactured by Wako Pure Chemical Industries, Ltd.) were added. The resultant mixture was stirred for 4 hours at room temperature. After the stirring was ended, water was added to the reaction solution, and extraction was performed by using dichloromethane. An organic layer was washed with a saturated saline solution, dried by using magnesium sulfate, and concentrated under a reduced pressure. Next, Flash chromatography (as an elution solution, 9/1 (v/v) of hexane/ethyl acetate solution was used) was performed, so that 14.5 g of the titled compound was obtained.

¹H-NMR (CDCl₃): 7.62 (2H, d, J=8.4), 7.32 (2H, m) 4.51 (2H, s), 3.44 (2H, m), 2.48 (2H, m), 1.43 (18H, s)

### Reference Example 18

### 3-(4-N-hydroxyamidinobenzyl-t-butoxycarbonylamino)propanoic acid t-butyl ester

To a methanol solution (200 mL) of 3-(4-cyanobenzyl-t-butoxycarbonylamino)propanoic acid t-butyl ester obtained in Reference Example 17, sodium bicarbonate (13.5 g, manufactured by Wako Pure Chemical Industries, Ltd.) and hydroxylamine hydrochloride (14.5 g, manufactured by Kanto Chemical Co., Inc) were added. The resultant mixture was heated and refluxed for 2 hours. After the stirring was ended, water was added to the reaction solution, and extraction was performed by using ethyl acetate. An organic layer was washed with a saturated saline solution, dried by using magnesium sulfate, and concentrated under a reduced pressure, so that the titled product was obtained. The obtained residue was directly used for the next reaction.

¹H-NMR (CDCl₃): 7.58 (2H, d, J=8.1), 7.27 (2H, m) 4.89 (2H, s), 4.70 (2H, m), 3.43 (2H, m), 2.47 (2H, s), 1.43 (18H, s)

### Reference Example 19

### 3-(4-(5-(4-cyclohexylphenyl)-1,2,4-oxadiazol-3-yl)benzyl-t-butoxycarbonylamino)propanoic acid t-butyl ester

To an N,N-dimethylformamide solution (1 mL) of 3-(4-N-hydroxyamidinobenzyl-t-butoxycarbonylamino) propanoic acid t-butyl ester (104.8 mg) obtained in Reference Example 18 and 4-cyclohexylbenzoic acid (51.1 mg, manufactured by Tokyo Chemical Industry Co., Ltd.), WSC hydrochloride (47.9 mg, manufactured by Watanabe Chemical Industries, Ltd.) and HOBt (33.8 mg, manufactured by Watanabe Chemical Industries, Ltd.) were added. The resultant mixture was stirred overnight at 90° C. After the stirring was ended, the reaction solution was poured into water, and extraction was performed by using dichloromethane. An organic layer was washed with a saturated saline solution, dried by using magnesium sulfate, and concentrated under a reduced pressure. Subsequently, chromatography (as an elution solution, 7:1 (v/v) of hexane/ethyl acetate was used) using Biotage 12M cartridge was performed, so that 52.6 mg of the titled compound was obtained.

¹H-NMR (CDCl₃): 8.13 (4H, d, J=9.0), 7.38 (4H, d, J=9), 4.52 (2H, s), 3.34-3.42 (2H, m), 2.41-2.60 (3H, m) 1.76-1.90 (4H, m), 1.25-1.51 (24H, m)

### Example 38

### 3-(4-(5-(cyclohexylphenyl)-1,2,4-oxadiazol-3-yl)benzylamino)propanoic acid

To 3-(4-(5-(Cyclohexylphenyl)-1,2,4-oxadiazol-3-yl)benzyl-t-butoxycarbonylamino)propanoic acid t-butyl ester (52.6 mg) obtained in Reference Example 19, 4N HCl dioxane solution (2 mL, manufactured by Kokusan Kagaku Company) was added. The resultant mixture was stirred overnight at room temperature. After the stirring was ended, the product was taken out by filtration, and dried under a reduced pressure, so that 23 mg of the titled compound was obtained. ESI-MS: 406 (M+H), RTime 4.21 min.

According to the procedures of Reference Example 19 and Example 38, the same method was performed except that any one of raw compounds listed in Table 74 was used instead of 4-cyclohexylbenzoic acid. As a result, the compounds of Examples 39 to 108 listed in Table 74 were obtained.

TABLE 74

LCMS(ESI+)

| Exp. | Syn. | SM. | Reagent | Structure | MASS | RT |
|---|---|---|---|---|---|---|
| 39 | P2 | 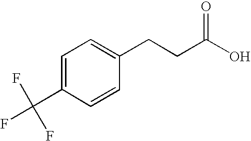 | Ald | 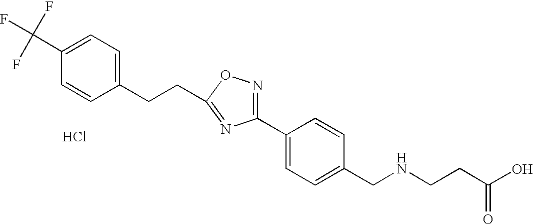 | 420.0 | 3.94 |
| 40 | P2 | 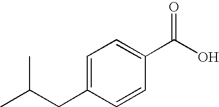 | TCI | 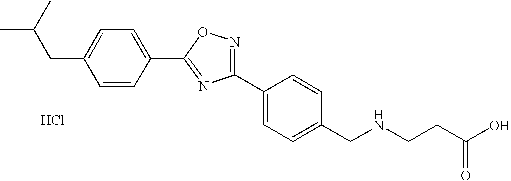 | 380.0 | 4.18 |
| 41 | P2 | 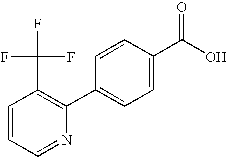 | Syn | 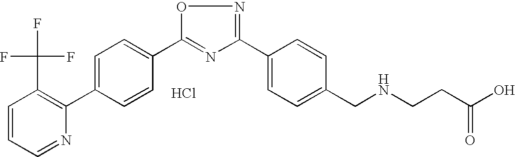 | 469.3 | 3.79 |
| 42 | P2 | 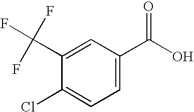 | TCI | 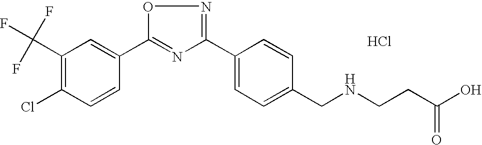 | 426.2 | 3.97 |
| 43 | P2 | 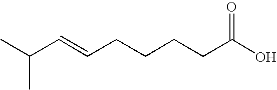 | May | 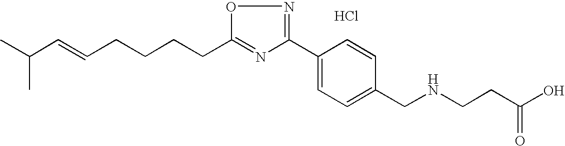 | 372.4 | 4.09 |
| 44 | P2 | 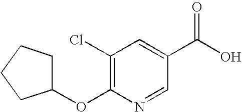 | Syn | 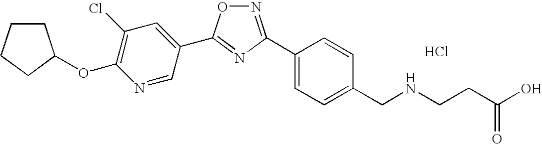 | 443.3 | 4.12 |
| 45 | P2 | 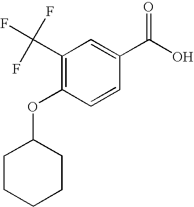 | Syn | 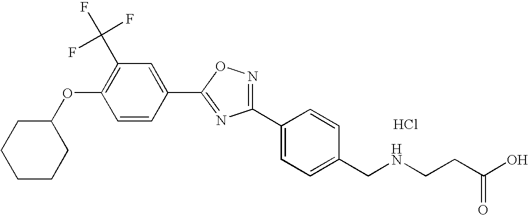 | 490.2 | 4.36 |
| 46 | P2 | 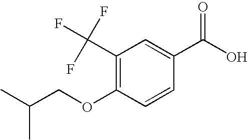 | Syn | 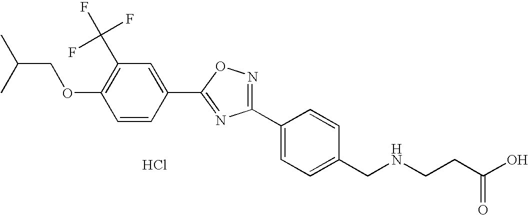 | 464.2 | 4.21 |
| 47 | P2 | 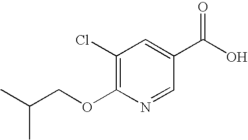 | Syn | 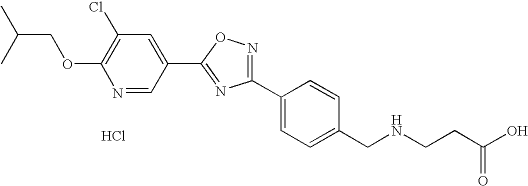 | 431.3 | 4.12 |
| 48 | P2 | 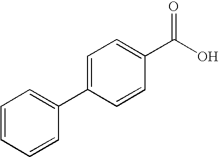 | TCI | 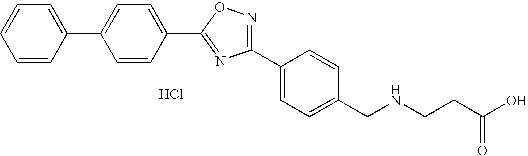 | 400.3 | 4.02 |
| 49 | P2 | 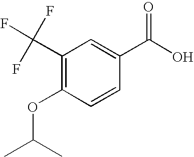 | Syn | 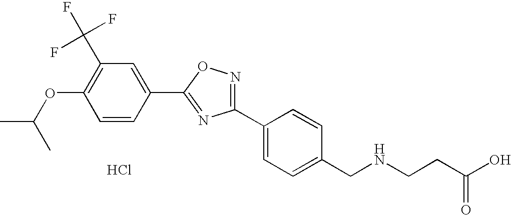 | 450.2 | 4.00 |
| 50 | P2 | 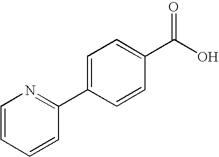 | WAKO | 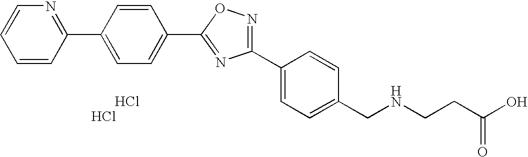 | 401.3 | 3.61 |
| 51 | P2 | 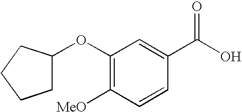 | MAY | 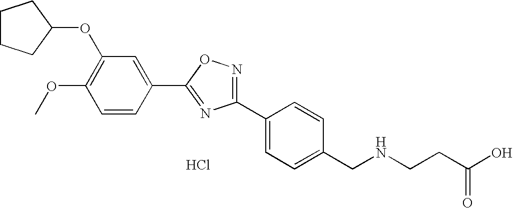 | 438.3 | 3.79 |
| 52 | P2 | 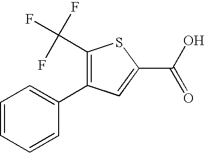 | May | 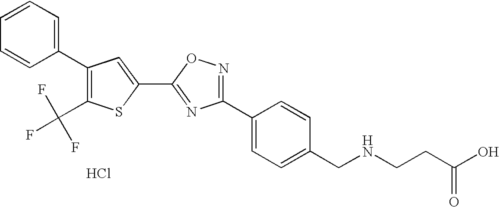 | 474.2 | 4.12 |
| 53 | P2 | 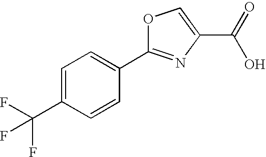 | Syn | 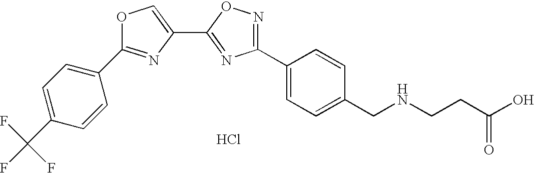 | 459.2 | 3.97 |
| 54 | P2 | 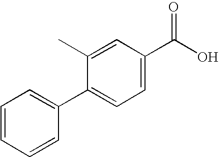 | Syn | 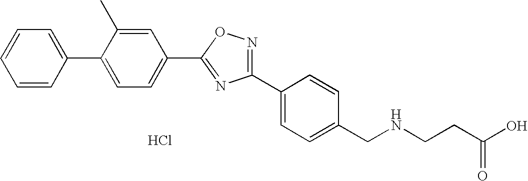 | 414.2 | 3.71 |
| 55 | P2 | 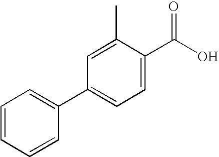 | Syn | 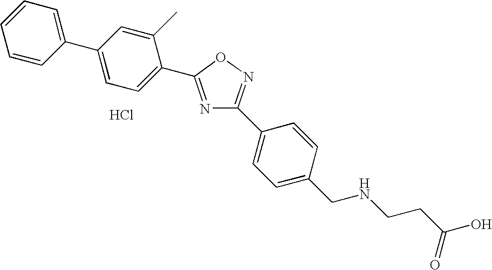 | 414.3 | 3.83 |
| 56 | P2 | 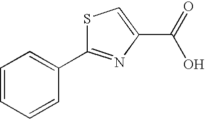 | Syn | 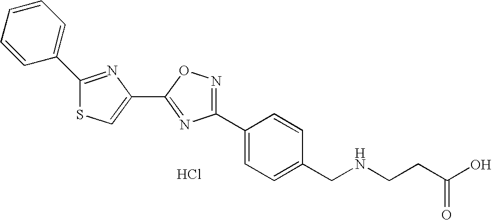 | 379.1 | 3.10 |
| 57 | P2 | 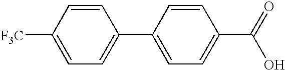 | Syn | 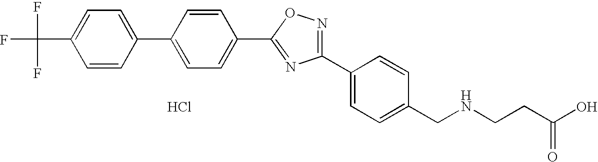 | 468.0 | 4.30 |
| 58 | P2 | 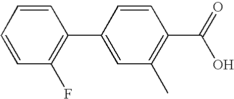 | Syn | 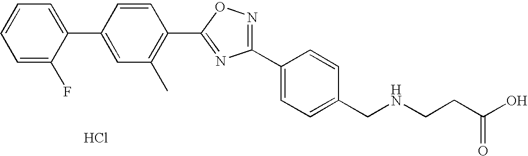 | 432.0 | 4.12 |
| 59 | P2 | 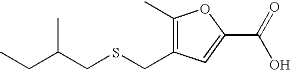 | Matrix | 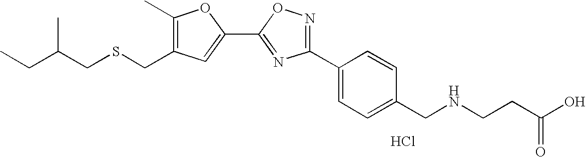 | 430.0 | 3.86 |
| 60 | P2 | 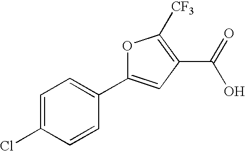 | May | 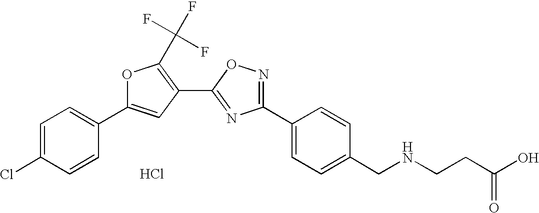 | 491.9 | 4.34 |
| 61 | P2 | 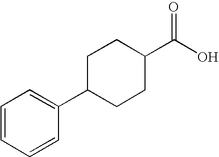 | Matrix | 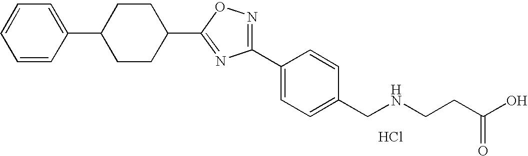 | 406.1 | 3.86 |
| 62 | P2 | 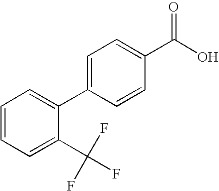 | Oak | 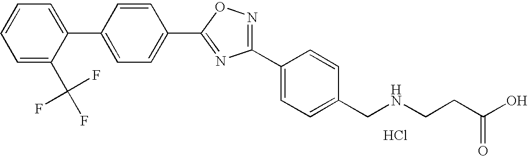 | 468.0 | 3.92 |
| 63 | P2 | 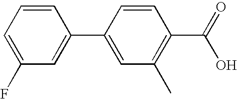 | Syn | 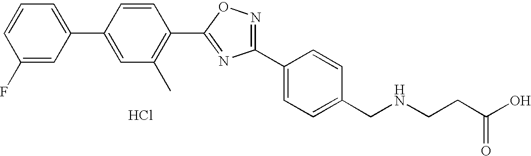 | 432.0 | 4.13 |
| 64 | P2 | 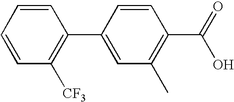 | Syn | 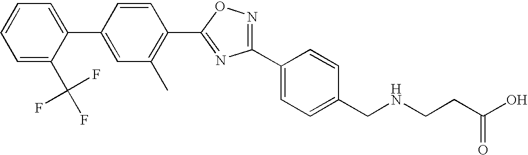 | 482.0 | 4.04 |
| 65 | P2 | 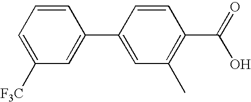 | Syn | 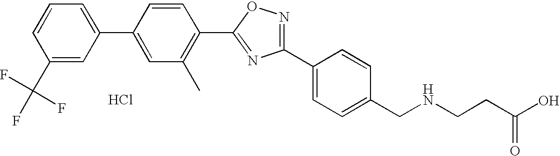 | 482.0 | 4.24 |
| 66 | P2 | 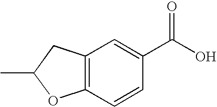 | Matrix | 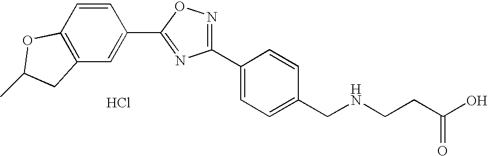 | 380.0 | 3.56 |
| 67 | P2 | 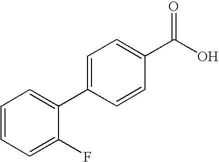 | May | 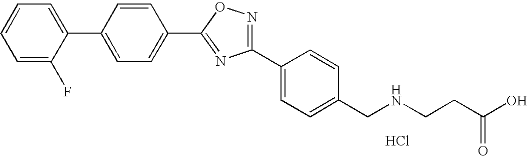 | 418.0 | 3.89 |
| 68 | P2 | 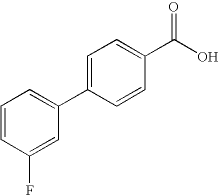 | May | 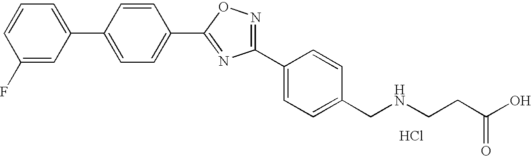 | 418.0 | 3.99 |
| 69 | P2 | 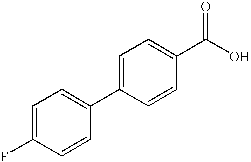 | May | 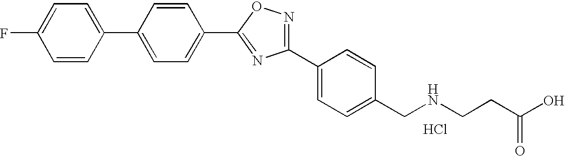 | 418.0 | 4.00 |
| 70 | P2 | 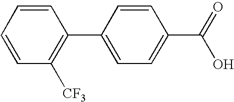 | Syn | 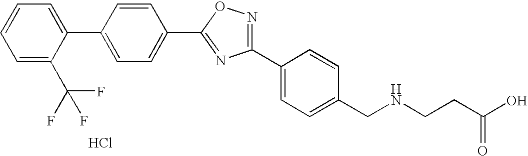 | 468.0 | 4.11 |
| 71 | P2 | 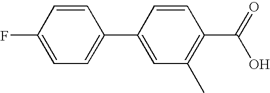 | Syn | 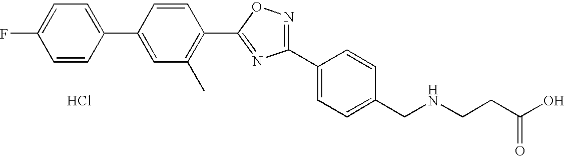 | 432.0 | 4.13 |
| 72 | P2 | 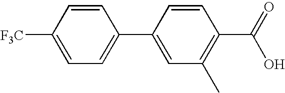 | Syn | 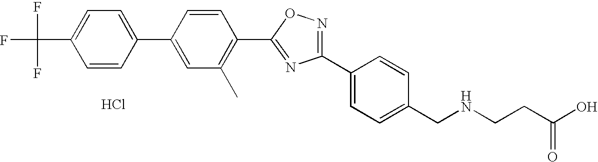 | 482.0 | 4.37 |
| 73 | P2 | 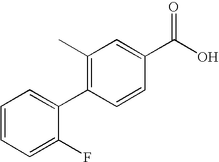 | Syn | 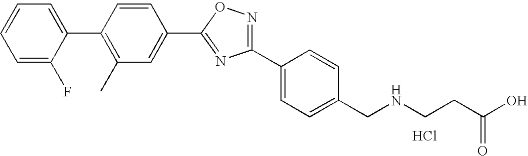 | 468.0 | 4.11 |
| 74 | P2 | 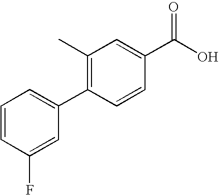 | Syn | 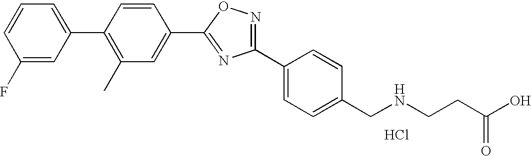 | 432.0 | 3.99 |
| 75 | P2 | 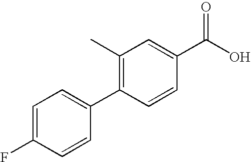 | Syn | 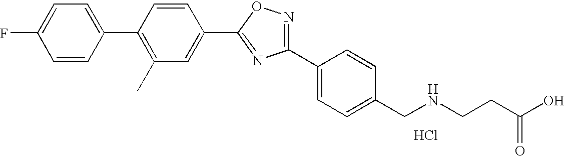 | 432.0 | 4.02 |
| 76 | P2 | 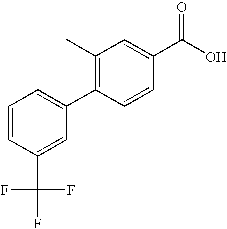 | Syn | 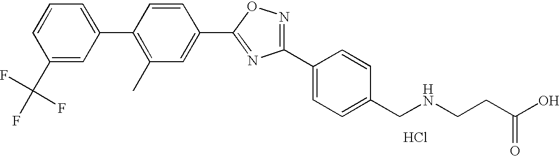 | 482.0 | 4.14 |
| 77 | P2 | 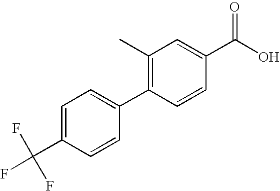 | Syn | 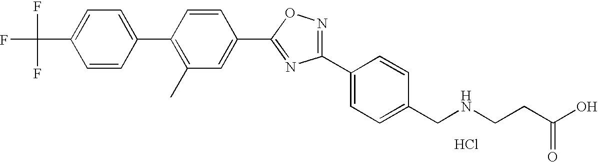 | 482.0 | 4.23 |
| 78 | P2 | 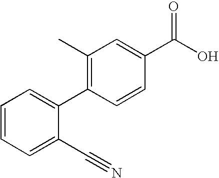 | Syn | 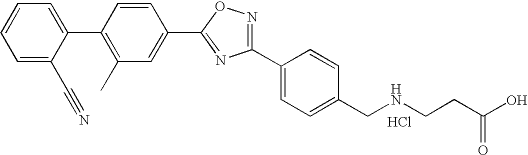 | 439.2 | 3.27 |
| 79 | P2 | 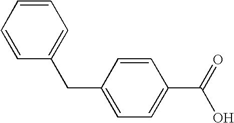 | ABCR | 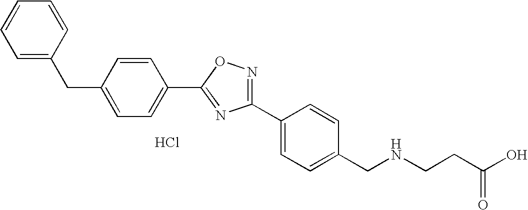 | 414.0 | 3.87 |
| 80 | P2 | 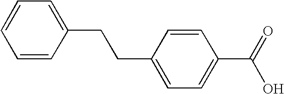 | Syn | 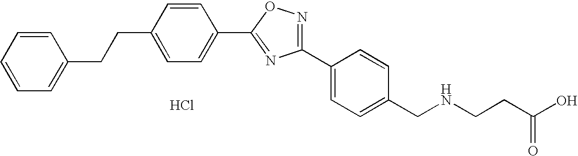 | 428.1 | 4.08 |
| 81 | P2 | 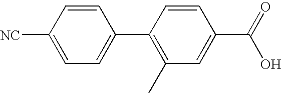 | Syn | 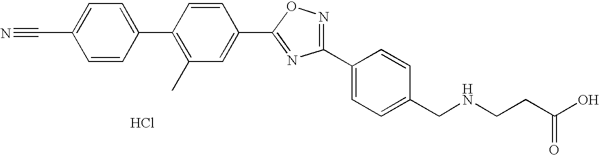 | 439.1 | 3.91 |
| 82 | P2 | 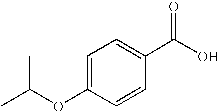 | Syn | 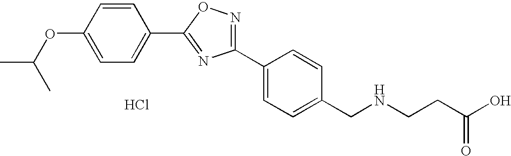 | 406.0 | 4.15 |
| 83 | P2 | 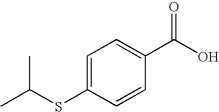 | Syn | 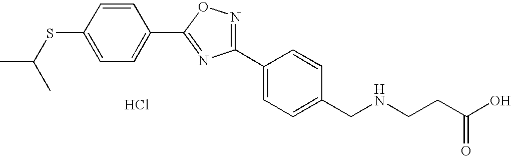 | 419.9 | 4.33 |
| 84 | P2 | 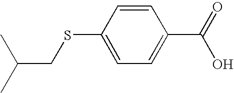 | Syn | 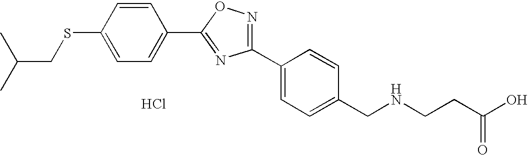 | 412.1 | 3.69 |
| 85 | P2 | 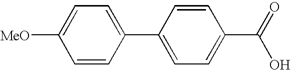 | BioNet | 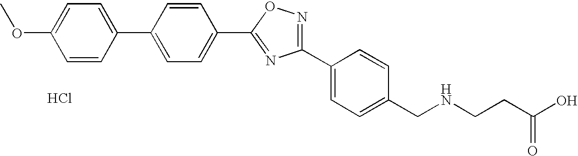 | 430.2 | 3.60 |
| 86 | P2 | 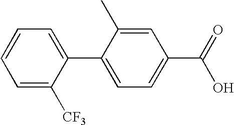 | Syn | 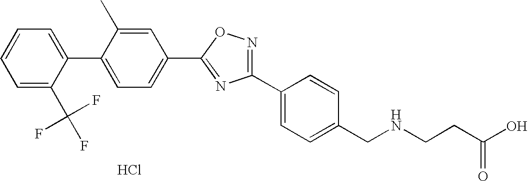 | 482.0 | 4.14 |
| 87 | P2 | 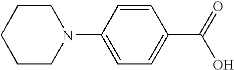 | May | 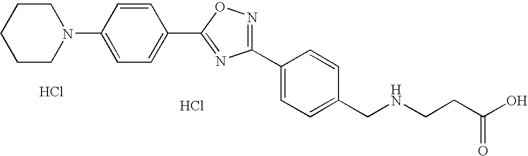 | 407.0 | 3.91 |
| 88 | P2 | 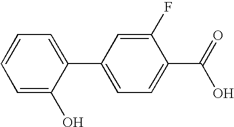 | Syn | 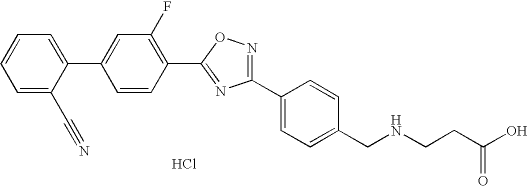 | 443.2 | 3.41 |
| 89 | P2 | 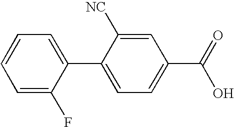 | Syn | 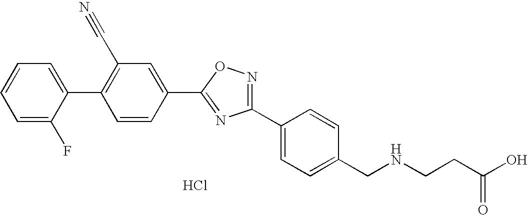 | 442.9 | 3.79 |
| 90 | P2 | 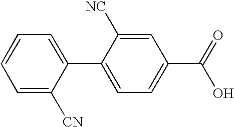 | Syn | 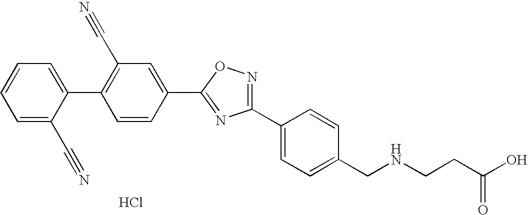 | 449.9 | 3.63 |
| 91 | P2 | 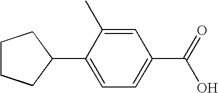 | Syn | 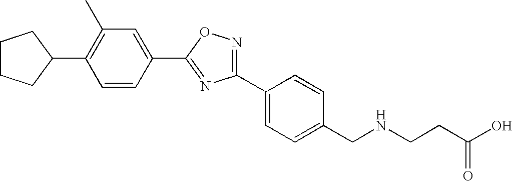 | 406.0 | 4.15 |
| 92 | P2 | 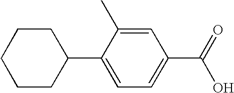 | Syn | 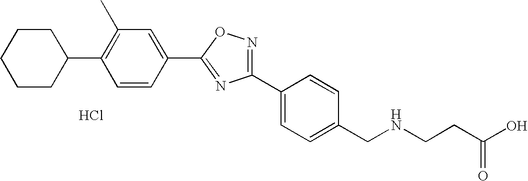 | 419.9 | 4.33 |
| 93 | P2 | 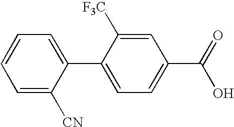 | Syn | 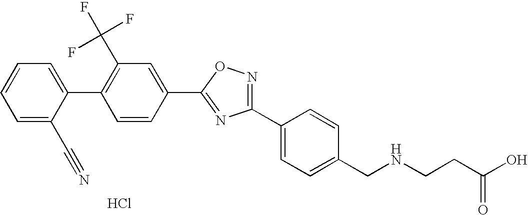 | 493.1 | 3.36 |
| 94 | P2 | 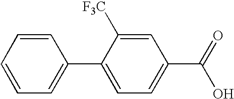 | Syn | 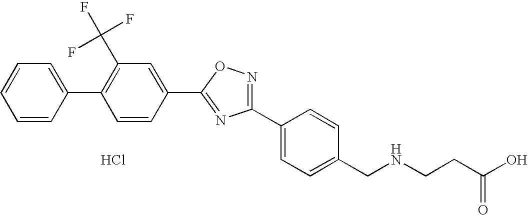 | 468.1 | 3.74 |
| 95 | P2 | 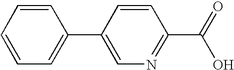 | SynChem. | 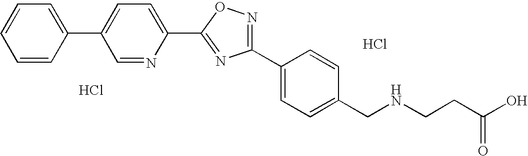 | 401.3 | 3.61 |
| 96 | P2 | 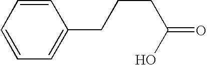 | TCI | 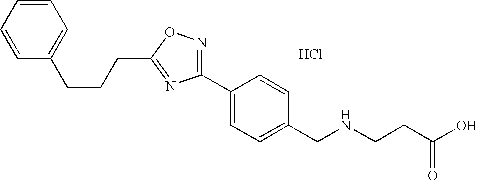 | 366.2 | 3.13 |
| 97 | P2 | 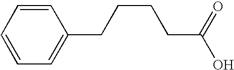 | TCI | 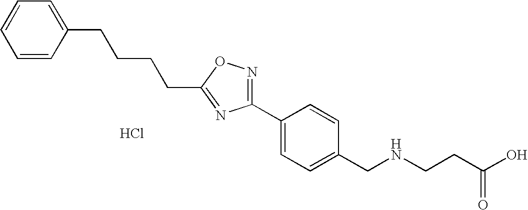 | 380.2 | 3.32 |
| 98 | P2 | 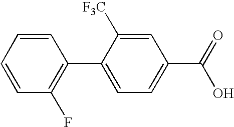 | Syn | 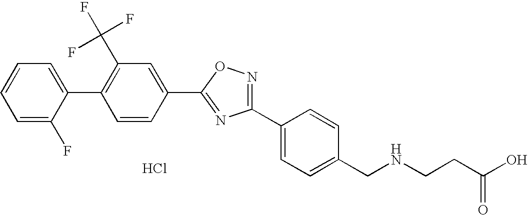 | 486.1 | 3.78 |
| 99 | P2 | 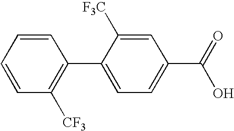 | Syn | 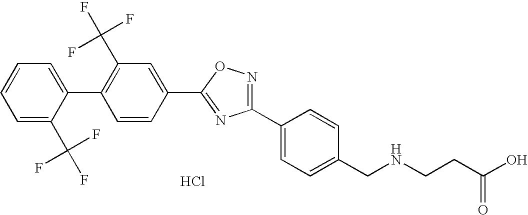 | 534.1 | 3.92 |
| 100 | P2 | 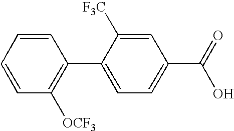 | Syn | 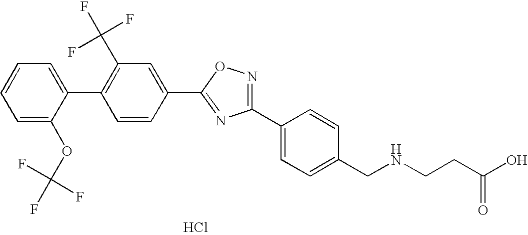 | 552.1 | 3.97 |
| 101 | P2 | 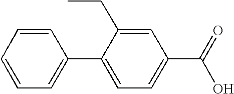 | Syn | 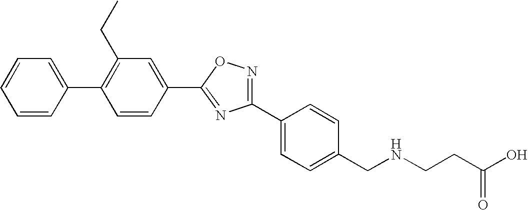 | 428.3 | 1.46 |
| 102 | P2 | 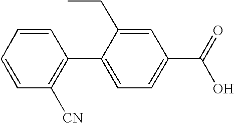 | Syn | 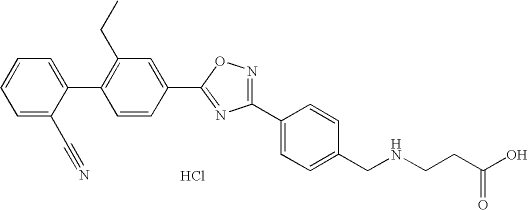 | 453.3 | 1.30 |
| 103 | P2 | 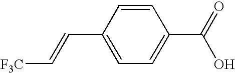 | Syn | 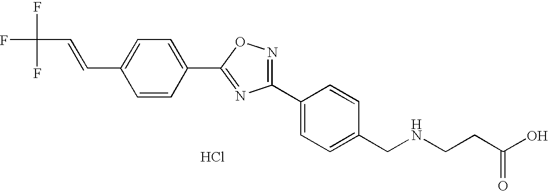 | 418.3 | 1.32 |
| 104 | P2 | 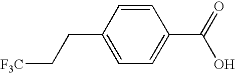 | Syn | 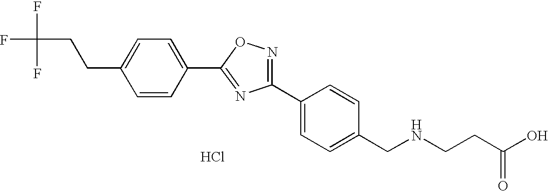 | 420.3 | 1.24 |
| 105 | P2 | 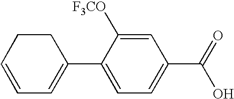 | Syn | 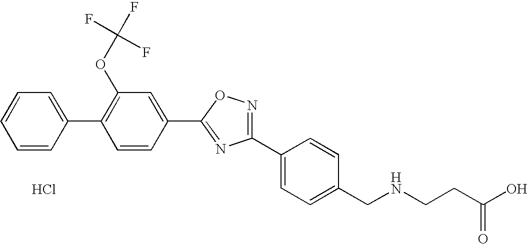 | 484.3 | 1.45 |
| 106 | P2 | 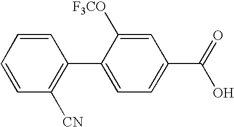 | Syn | 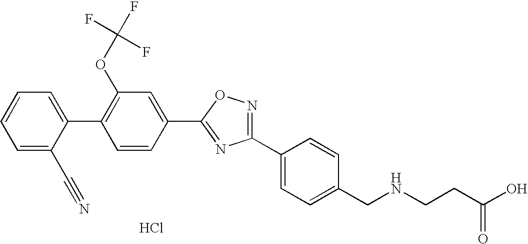 | 509.3 | 1.33 |
| 107 | P2 | 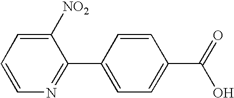 | Syn | 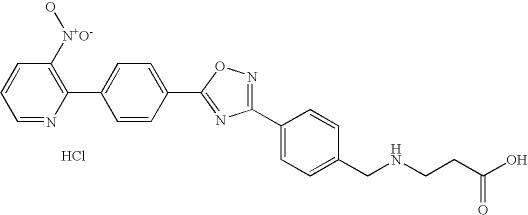 | 446.3 | 1.10 |
| 108 | P2 | 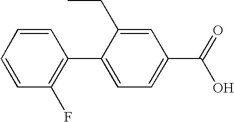 | Syn | 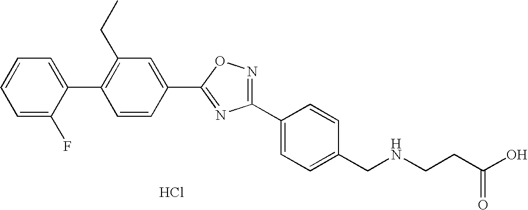 | 446.3 | 1.42 |

Among the “Reagents” listed in Table 74, the compounds denoted by “Syn” may be obtained by using the following synthesizing methods.

### Reference Example 20

### 4-isobutoxy-3-(trifluoromethyl)benzoic acid

To an N,N-dimethylformamide solution (2 mL) of 4-fluoro-3-(trifluoromethyl)benzoic acid (100 mg, manufactured by Fluorochem) and isobutanol (118 mg, manufactured by Tokyo Chemical Industry Co., Ltd.), sodium hydride (G2 mg, manufactured by Wako Pure Chemical Industries, Ltd.) was added. The resultant mixture was stirred for 3 hours at 80° C. After the stirring was ended, 1N HCl aqueous solution (2 mL, manufactured by Wako Pure Chemical Industries, Ltd.) was added to the reaction solution, and extraction was performed by using dichloromethane. An organic layer was dried by using magnesium sulfate, and concentrated under a reduced pressure, so that the titled compound (155 mg) was obtained. ESI-MS: 261.2 (M−H), RTime 4.80 min.

### Reference Example 21

### 4-(cyclohexyloxy)-3-(trifluoromethyl)benzoic acid

To an N,N-dimethylformamide solution (2 mL) of 4-fluoro-3-(trifluoromethyl)benzoic acid (100 mg, manufactured by Fluorochem) and cyclohexanol (159 mg, manufactured by Tokyo Chemical Industry Co., Ltd.), sodium hydride (62 mg, manufactured by WakoPure Chemical Industries, Ltd.) was added. The resultant mixture was stirred for 3 hours at 80° C. After the stirring was ended, 1N HCl aqueous solution (2 mL, manufactured by Wako Pure Chemical Industries, Ltd.) was added to the reaction solution, and extraction was performed by using dichloromethane. An organic layer was dried by using magnesium sulfate, and concentrated under a reduced pressure, so that the titled compound (155 mg) was obtained. ESI-MS: 287.2 (M−H), RTime 5.00 min.

### Reference Example 22

### 4-isopropoxy-3-(trifluoromethyl)benzoic acid

To an N,N-dimethylformamide solution (3 mL) of 4-fluoro-3-(trifluoromethyl)benzoic acid (112 mg, manufactured by Fluorochem) and isopropanol (63.6 mg, manufactured by Tokyo Chemical Industry Co., Ltd.), sodium hydride (63.6 mg, manufactured by Wako Pure Chemical Industries, Ltd.) was added. The resultant mixture was stirred for 3 hours at 100° C. After the stirring was ended, 1N HCl aqueous solution (2 mL, manufactured by Wako Pure Chemical Industries, Ltd.) was added to the reaction solution, and extraction was performed by using dichloromethane. An organic layer was dried by using magnesium sulfate, and concentrated under a reduced pressure, so that the titled compound (159 mg) was obtained. ESI-MS: 247.2 (M−H), RTime 4.47 min.

### Reference Example 23

### 5-chloro-6-isobutoxynicotinic acid

To an N,N-dimethylformamide solution (10 mL) of 5,6-dichloronicotinic acid (500 mg, manufactured by Tokyo Chemical Industry Co., Ltd) and isobutanol (716 μL, manufactured by Tokyo Chemical Industry Co., Ltd), sodium hydride (312 mg, manufactured by Wako Pure Chemical Industries, Ltd.) was added. The resultant mixture was stirred for 40 minutes at room temperature, and stirred for 3 hours at 120° C. After the stirring was ended, the reaction solution was poured into water (35 mL), and acetic acid was added thereto to adjust the pH of the solution at 5. A product was taken by filtration and rinsed with water and hexane, and dried under a reduced pressure, so that the titled compound (372 mg) was obtained. ¹H-NMR (CDCl₃): 8.75 (1H, d, J=2.1), 8.25 (1H, d, J=2.1) 4.24 (2H, d, J=6.9), 2.10-2.24 (1H, m), 1.06 (6H, d, J=6.9),

### Reference Example 24

### 5-chloro-6-(cyclohexyloxy)nicotinic acid

To an N,N-dimethylformamide solution (10 mL) of 5,6-dichloronicotinic acid (500 mg, manufactured by Tokyo Chemical Industry Co., Ltd) and cyclohexanol (784 μL, manufactured by Tokyo Chemical Industry Co., Ltd), sodium hydride (312 mg, manufactured by Wako Pure Chemical Industries, Ltd.) was added. The resultant mixture was stirred for 40 minutes at room temperature, and stirred for 3 hours at 120° C. After the stirring was ended, the reaction solution was poured into water (35 mL), acetic acid was added thereto to adjust the pH of the solution at 5. A product was taken by filtration and rinsed with water and hexane, and dried under a reduced pressure, so that the titled compound (553 mg) was obtained. 1H-NM R (CDCl₃): 8.73 (1H, s), 8.21 (1H, s), 5.19-5.27 (1H, m), 1.41-1.98 (10H, m)

### Reference Example 25

### 4-(isopropylthio)benzoic acid

### Process 1

### 4-(isopropylthio)Benzoic Acid Methyl Ester

To an N,N-dimethylformamide solution (3 mL) of 4-mercaptobenzoic acid methyl ester (200 mg, manufactured by Tokyo Chemical Industry Co., Ltd.) and potassium carbonate (328.9 mg, manufactured by Kokusan Kagaku Company), 1-iodo-2-methylpropane (327.6 mg, manufactured by Tokyo Chemical Industry Co., Ltd.) was added. The resultant mixture was stirred overnight at room temperature. After the stirring was ended, the reaction solution was poured into water, and extraction was performed by using diethyl ether. An organic layer was washed with a saturated saline solution, dried by using magnesium sulfate, and concentrated under a reduced pressure, so that the titled compound (122.2 mg) was obtained. ¹H-NMR (CDCl₃): 7.92 (2H, d, J=1.8), 7.35 (2H, d, J=1.8), 3.90 (3H, s), 3.49-3.59 (1H, m), 1.34-1.37 (6H, brs)

### Process 2

### 4-(isopropylthio)Benzoic Acid

To an ethanol solution (5 mL) of 4-(isopropylthio)benzoic acid methyl ester (122.2 mg), 5N sodium hydroxide aqueous solution (1 mL, manufactured by Wako Pure Chemical Industries, Ltd.) was added. The resultant mixture was stirred for 5 hours at room temperature. After the stirring was ended, 5N HCl aqueous solution (2 mL, manufactured by Wako Pure Chemical Industries, Ltd.) was added thereto, and extraction was performed by using chloroform. An organic layer was dried by using magnesium sulfate, and concentrated under a reduced pressure, so that the titled compound (105 mg) was obtained.

### Reference Example 26

### 4-cyclopentyl-3-methyl benzoic acid

### Process 1

### 4-cyclopentenyl-3-methyl benzoic acid methyl ester

To a dioxane solution (5 mL) of cyclopenten-1-ylboronic acid (134.2 mg, manufactured by CombiBlock) and 4-bromo-3-methylbenzoic acid methyl ester, trisbenzylidene acetone dipalladium (45.8 mg, manufactured by Aldrich), tri-t-butylphosphine tetraphenylboronate (62.7 mg, manufactured by Kanto Chemical Co., Inc), and cesium carbonate (651.6 mg, manufactured by Kanto Chemical Co., Inc) were added. The resultant mixture was stirred for 16 hours at 90° C. After the stirring was ended, the reaction solution was filtered by using Celite and the filtrate was concentrated under a reduced pressure. For the obtained residue, flash chromatography (as an elution solution, 20:1 (v/v) of hexane/ethyl acetate was used) was performed, so that 291.7 mg of the titled compound was obtained.

¹H-NMR (CDCl₃): 7.80 (1H, s), 7.09-7.26 (2H, m), 5.85-5.88 (1H, brs), 3.93 (3H, s), 2.65-2.72 (3H, m) 2.52-2.59 (3H, m)/2.40 (3H, s).

### Process 2

### 4-cyclopentyl-3-methylbenzoic Acid Methyl Ester

To a methanol solution (5 mL) of 4-cyclopentenyl-3-methylbenzoic acid methyl ester obtained in Process 1, 10% palladium carbon (30 mg, manufactured by NE Chemcat Corp.) was added. The resultant mixture was stirred for 5 hours at room temperature under a hydrogen ambience. After the stirring was ended, the reaction solution was filtered by using Celite, and the filtered solution was concentrated, so that the titled compound was obtained.

¹H-NMR (CDCl₃): 7.80 (1H, s), 7.09-7.31 (2H, m), 3.91 (3H, s), 3.22 (1H, m), 2.38 (3H, s), 2.00-2.07 (2H, m), 1.55-1.86 (6H, m)

### Process 3

### 4-cyclopentyl-3-methylbenzoic Acid

To an ethanol solution (5 mL) of 4-cyclopentyl-3-methylbenzoic acid methyl ester obtained in Process 2, a 5N sodium hydroxide aqueous solution (1 mL, manufactured by Wako Pure Chemical Industries, Ltd.) was added. The resultant mixture was stirred overnight at room temperature. After the stirring was ended, a 5N HCl aqueous solution (2 mL, manufactured by Wako Pure Chemical Industries, Ltd.) was added to there action solution. A precipitate was taken by filtration and rinsed with hexane and dried under a reduced pressure, so that 123 mg of the titled compound was obtained. ESI-NS: 203.2 (M−H), RTime 4.49 min.

### Reference Example 27

### 4-cyclohexyl-3-methylbenzoic acid

According to the procedures of Reference Example 26, the same method was performed except that cyclohexen-1-ylboronic acid was used instead of cyclopenten-1-ylboronic acid, so that the titled compound was obtained. ESI-MS: 217.2 (M−H), RTime 4.76 min.

### Reference Example 28 (Syn. X)

### 4-phenyl-3-methylbenzoic acid

To an N,N-dimethylformamide solution (4.0 mL) of phenylboronic acid (36.6 mg, manufactured by Tokyo Chemical Industry Co., Ltd.) and 4-bromo-3-methylbenzoic acid (43.0 mg, manufactured by Wako Pure Chemical Industries, Ltd.), [1,1′-bis(diphenylphosphino)ferrocene]dichloro palladium, dichloromethane complex (1:1) (49.0 mg, manufactured by Aldrich), cesium carbonate (163 mg, manufactured by Wako Pure Chemical Industries, Ltd.) were added. The resultant mixture was stirred for 16 hours at 80° C. After the stirring was ended, the reaction solution was concentrated, 1NHCl aqueous solution (manufactured by Wako Pure Chemical Industries, Ltd.) was added thereto, extraction was performed by using ethyl acetate, and an organic layer was dried by using magnesium sulfate. For the obtained residue, flash chromatography (as an elution solution, 2:1 (v/v) of hexane/ethyl acetate was used) was performed, so that the titled compound (14.9 mg) was obtained. ESI-MS: 211.2 (M−H), RTime 4.53 min.

### Reference Example 29 (Syn. Y)

### 4-benzylbenzoic acid

To a THF/water mixture solution (5 mL/0.5 mL) of potassium benzyltrifluoroborate (120 mg, manufactured by Aldrich) and 4-bromobenzoic acid (100 mg, manufactured by Wako Pure Chemical Industries, Ltd.), [1,1′-bis(diphenylphosphino)ferrocene] dichloro palladium, dichloromethane complex (1:1) (41.2 mg, manufactured by Aldrich) and cesium carbonate (492 mg, manufactured by Wako Pure Chemical Industries, Ltd.) were added. The resultant mixture was refluxed for 19 hours. After the stirring was ended, the reaction solution was concentrated, 1N HCl aqueous solution (manufactured by Wako Pure Chemical Industries, Ltd.) was added thereto, extraction was performed by using ethyl acetate, and an organic layer was dried by using magnesium sulfate. For the obtained residue, flash chromatography (as an elution solution, 6:1 (v/v) of hexane/ethyl acetate was used) was performed, so that the titled compound (80.6 mg) was obtained. ESI-MS: 211.2 (M−H, RTime 4.53 min. ESI-NS: 211.2 (M−H), RTime 4.62 min

### Reference Example 30 (Syn. Z)

### Process 1

### 2′-cyano-2-(trifluoromethyl)-biphenyl-4-carboxylic acid methyl ester

To a 1,4-dioxane solution (5.0 mL) of cyanophenylboronic acid (220 mg, manufactured by Aldrich) and 4-chloro-3-(trifluoromethyl)benzoic acid methyl (238 mg), trisbenzylideneacetone dipalladium (91.4 mg, manufactured by Aldrich), tri-t-butylphosphine tetraphenylboronate (130 mg, manufactured by Kanto Chemical Co., Inc), and cesium carbonate (489 mg, manufactured by Kanto Chemical Co., Inc) were added. The resultant mixture was stirred for 18 hours at 90° C. After the stirring was ended, the reaction solution was filtered by using Celite, and the filtered solution concentrated under a reduced pressure. For the obtained residue, flash chromatography (as an elution solution, 20:1 (v/v) of hexane/ethyl acetate was used) was performed, so that the titled compound was obtained. ¹H-NMR (CDCl₃): 8.48 (1H, dd, J=0.5, J=1.1), 8.30 (1H, ddd, J=0.5, J=1.3, J=7.8), 7.77 (1H, ddd, J=0.5, J=1.3, J=7.8), 7.65 (1H, ddd, J=1.3, J=7.8, J=7.8), 7.54 (1H, ddd, J=1.3, J=7.8, J=7.8), 7.47 (1H, brd, J=7.8), 7.41 (1H, brd, J=7.8), 3.99 (3H, s)

### Process 2

### 2′-cyano-2-(trifluoromethyl)-biphenyl-4-carboxylic acid

To a methanol solution (5.0 mL) of 2-cyano-2-(trifluoromethyl)-biphenyl-4-carbonic acid methyl ester obtained in Process 1, 5N sodium hydroxide aqueous solution (581 μL, manufactured by Wako Pure Chemical Industries, Ltd.) was added. The resultant mixture was stirred for 16 hours. After the stirring was ended, 1N HCl aqueous solution was added to the reaction solution, a product was taken by filtration, and dried under a reduced pressure, so that 265 mg of the titled compound was obtained. ESI-MS: 406 (M−H), RTime 3.95 min. According to the procedures of Syn. X to Syn. Z, the same method was performed except that any one of raw compounds are listed in Table 75 was used. As a result, the carboxylic acids listed in Table 75 were synthesized.

TABLE 75

| | | | | | | | LCMS (ESI−) | |
|---|---|---|---|---|---|---|---|---|
| Ref. | Syn. | SM | Reagent | SM | Reagent | Structure | MASS | RT |
| 31 | X | 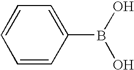 | TCI | 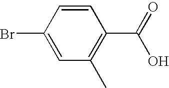 | LAN | 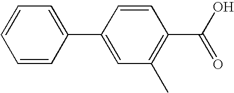 | 211.2 | 4.56 |
| 32 | X | 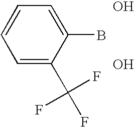 | TCI | 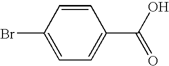 | WAKO | 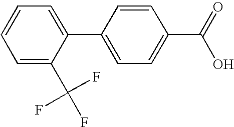 | 265.2 | 4.68 |
| 33 | X | 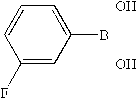 | WAKO | 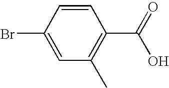 | LANr | 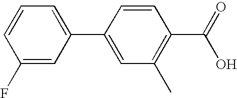 | 229.2 | 4.69 |
| 34 | X | 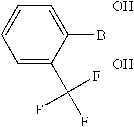 | TCI | 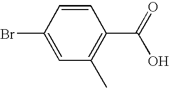 | LAN | 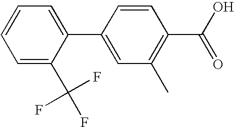 | 279.2 | 4.83 |
| 35 | X | 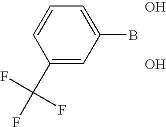 | Fluka | 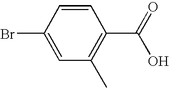 | LAN | 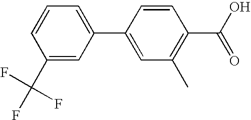 | 279.3 | 4.98 |
| 36 | X | 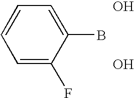 | Ald | 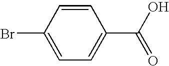 | WAKO | 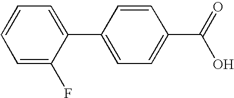 | 215.2 | 4.47 |
| 37 | X | 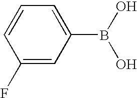 | WAKO | 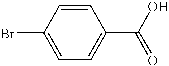 | WAKO | 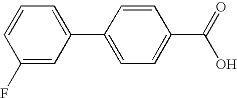 | 215.2 | 4.56 |
| 38 | X | 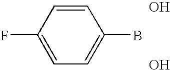 | TCI | 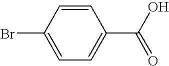 | WAKO | 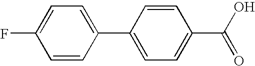 | 215.2 | 4.57 |
| 39 | X | 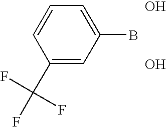 | Fluka | 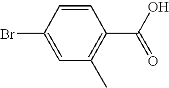 | LAN | 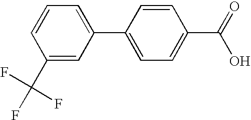 | 265.2 | 4.86 |
| 40 | X | 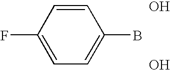 | TCI | 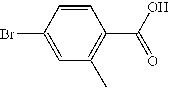 | LAN | 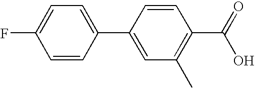 | 229.2 | 4.74 |
| 41 | X | 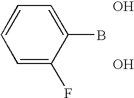 | Ald | 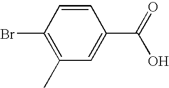 | WAKO | 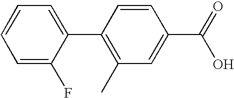 | 229.2 | 4.56 |
| 42 | X | 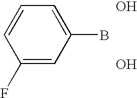 | WAKO | 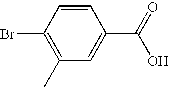 | WAKO | 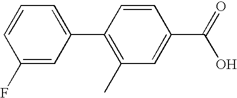 | 229.2 | 4.64 |
| 43 | X | 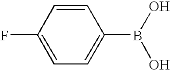 | TCI | 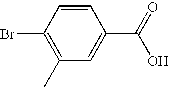 | WAKO | 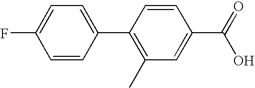 | 229.2 | 4.64 |
| 44 | X | 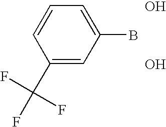 | Fluka | 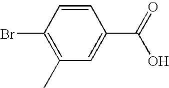 | WAKO | 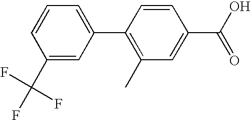 | 279.2 | 4.92 |
| 45 | X | 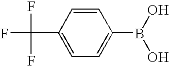 | WAKO | 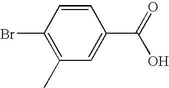 | WAKO | 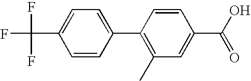 | 279.2 | 4.98 |
| 46 | X | 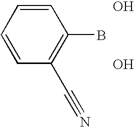 | Combi-BlocksInc. | 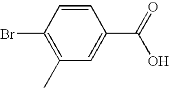 | WAKO | 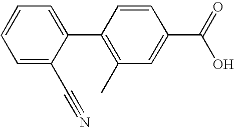 | 236.2 | 4.26 |
| 47 | Y | 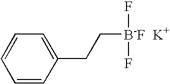 | Ald | 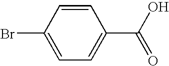 | WAKO | 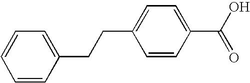 | 225.2 | 4.74 |
| 48 | X | 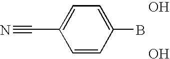 | Combi-BlocksInc. | 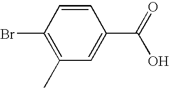 | WAKO | 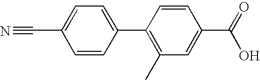 | 236.2 | 4.38 |
| 49 | X | 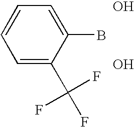 | Fluka | 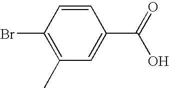 | WAKO | 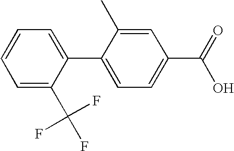 | 279.1 | 4.42 |
| 50 | Z | 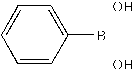 | TCI | 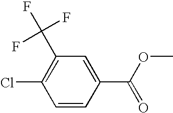 | Syn | 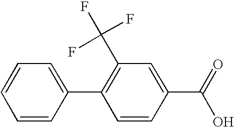 | 265.2 | 4.37 |
| 51 | Z | 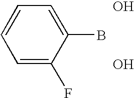 | WAKO | 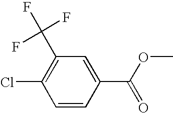 | Syn | 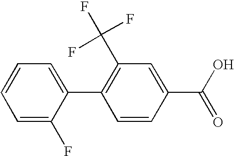 | 283.2 | 4.28 |
| 52 | Z | 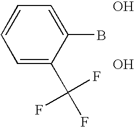 | Fluka | 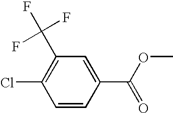 | Syn | 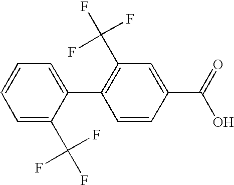 | 333.1 | 4.65 |
| 53 | Z | 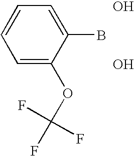 | WAKO | 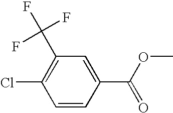 | Syn | 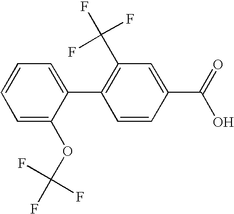 | 349.1 | 4.84 |
| 54 | Z | 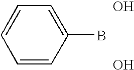 | TCI | 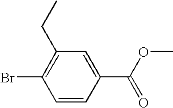 | Syn | 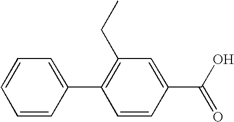 | 225.1 | 4.70 |
| 55 | Z | 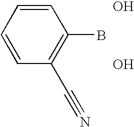 | Ald | 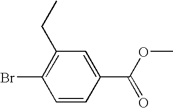 | Syn | 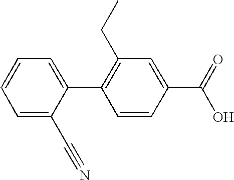 | 250.1 | 4.23 |
| 56 | Z | 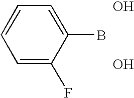 | WAKO | 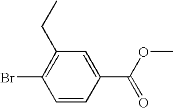 | Syn | 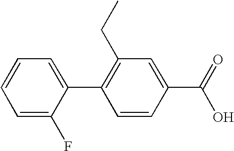 | 243.1 | 4.65 |
| 57 | Z | 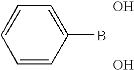 | TCI | 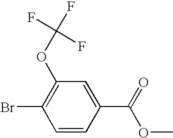 | Syn | 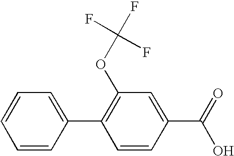 | 281.1 | 5.16 |
| 58 | Z | 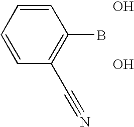 | Ald | 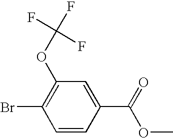 | Syn | 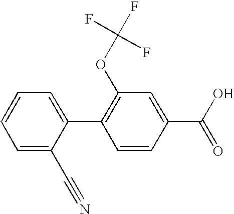 | 306.0 | 4.65 |
| 59 | Z | 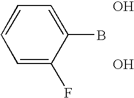 | WAKO | 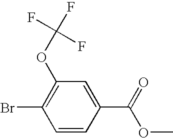 | Syn | 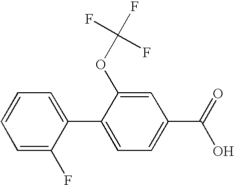 | 299.1 | 5.03 |
| 60 | Z | 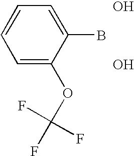 | WAKO | 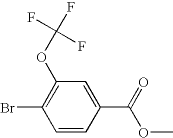 | Syn | 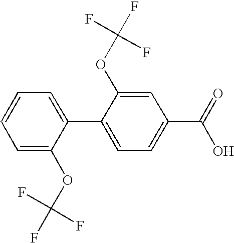 | 365.1 | 5.35 |
| 61 | Z | 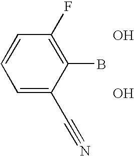 | Syn | 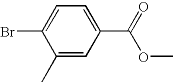 | TCI | 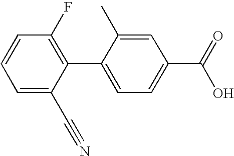 | 254.1 | 3.67 |
| 62 | Z | 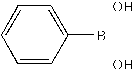 | TCI | 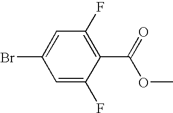 | Syn | 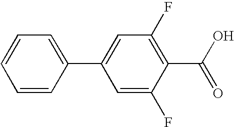 | 233.0 | 3.85 |
| 63 | Z | 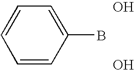 | TCI | 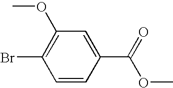 | Syn | 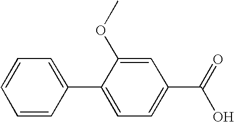 | 227.1 | 1.50 |
| 64 | Z | 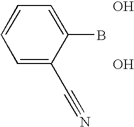 | Ald | 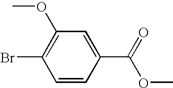 | Syn | 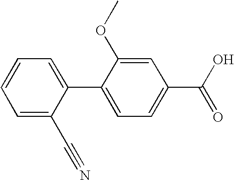 | 252.1 | 1.33 |

The boronic acid used in synthesis of Reference Example 61 was synthesized based on a method disclosed in the document of J. Org. Chem., Vol. 70, NO. 15, p. 5938 (2005). Among the “Reagent” listed in Table 75, the compounds denoted by “Syn” may be obtained by using the following synthesizing methods.

### Reference Example 65

### Process 1 4-chloro-3-(trifluoromethyl)benzoic acid

To a 2-methoxy ethanol solution (20 mL) of 2-chloro-5-cyanobenzotrifluoride (1.0 g, manufactured by Fluorochem), 2.5N sodium hydroxide aqueous solution (20 mL, manufactured by Wako Pure Chemical Industries, Ltd.) was added. The resultant mixture was stirred for 15 hours at 90° C. After the stirring was ended, 1N HCl aqueous solution (manufactured by Wako Pure Chemical Industries, Ltd.) was added to the reaction solution, extraction was performed by using ethyl acetate, and an organic layer was dried by using sodium sulfate. Subsequently, the organic layer was concentrated under a reduced pressure, so that the titled compound was obtained. The obtained residue was directly used for the next reaction. ESI-MS: 222.9 (M−H), RTime 3.95 min

### Process 2 4-chloro-3-(trifluoromethyl)benzoic acid methyl ester

To a methanol solution (49 mL) of 4-chloro-3-(trifluoromethyl)benzoic acid obtained in Process 1, a concentrated hydrochloric acid (11.0 mL) was added. The resultant mixture was stirred and refluxed for 4 hours. After the stirring was ended, the reaction solution was concentrated. The product was poured into a saturated sodium bicarbonate solution, and extraction was performed by using ethyl acetate. An organic layer was washed with a saturated saline solution, dried by using sodium sulfate, and further concentrated under a reduced pressure. Subsequently, chromatography (as an elution solution, 20:1 (v/v) of hexane/ethyl acetate was used) using Biotage 12s cartridge was performed, so that 983 mg of the titled compound was obtained. ¹H-NMR (CDCl₃): 8.36 (1H, d, J=2.0), 8.13 (1H, dd, J=2.0, J=8.4), 7.60 (1H, d, J=8.4), 3.96 (3H, s)

### Reference Example 66

### Process 1 4-bromo-3-(trifluoromethoxy)benzoic acid

To an acetnitrile solution (25 mL) of 4-amino-3-(trifluoromethoxy)benzonitrile (500 mg, manufactured by Fluorochem), copper (II) bromide (825 mg, manufactured by Kanto Chemical Co., Inc) and t-butyl nitrite (445 μL, manufactured by Acros Chemical Company) were added at −20° C. The resultant mixture was stirred for 1 hour at −20° C., and further stirred for 1 hour at room temperature. After the stirring was ended, the reaction solution was poured into water, and extraction was performed by using ethylacetate. An organic layer was washed with a saturated saline solution, and dried by using sodium sulfate. For the obtained residue, flash chromatography (as an elution solution, 1:0 (v/v) of hexane/ethyl acetate was used) was performed, so that 4-bromo-3-(trifluoromethoxy)benzonitrile (573 mg) was obtained. Next, hydrolysis thereof was performed by using the same method as that of Process 1 of Reference Example 65, so that the titled compound was obtained. The obtained residue was directly used for the next reaction. ESI-MS: 282.9 (M−H), RTime 4.23 min

### Process 2 4-bromo-3-(trifluoromethoxy)benzoic acid methyl ester

4-Bromo-3-(trifluoromethoxy)benzoic acid obtained in Process 1 was methyl-esterified by using the same method as that of Process 2 of Reference Example 65, so that 516 mg of the titled compound was obtained. ¹H-NMR (CDCl₃): 7.95 (1H, m), 7.84 (1H, dd, J=2.0, J=8.4), 7.73 (1H, d, J=8.4), 3.94 (3H, s)

### Reference Example 67

### 4-bromo-3-ethyl-benzoic acid methyl ester

4-Amino-3-ethylbenzonitrile (250 mg, manufactured by Alfa Aesar) was used for the same method as that of Reference Example 66, so that 232 mg of the titled compound was obtained. ¹H-NMR (CDCl₃): 7.90 (1H, d, J=2.1), 7.70 (11, dd, J=2.1, J=8.3), 7.59 (1H, d, J=8.3), 3.91 (3H, s), 2.80 (2H, q, J=7.5), 1.26 (3H, t, J=7.5)

### Reference Example 68

### 4-bromo-2,6-difluoro-benzoic acid methyl ester

4-Bromo-2,6-difluoro-benzoic acid (1.0 g, manufactured by Apollo Company) was methyl-esterified by using the same method as that of Process 2 of Reference Example 65, so that 594 mg of the titled compound was obtained. ¹H-NMR (CDCl₃): 7.16 (2H, dd, J=1.5, J=8.6), 3.94 (3H, s)

### Reference Example 69

### 4-bromo-3-methoxy-benzoic acid methyl ester

4-Amino-3-methoxy-benzoic acid (500 mg, manufactured by Aldrich) was brominated by using the same method as that of Process 1 of Reference Example 66, and methyl-esterification was performed by using the same method as that of Process 2 of Reference Example 65, so that 112 mg of the titled compound was obtained. 1H-NMR (CDCl₃): 7.60 (1H, d, J=8.1), 7.53 (1H, dd, J=1.8, J=8.1), 7.49 (1H, d, J=1.8), 3.95 (3H, s), 3.92 (3H, s)

### Reference Example 70

### 3-(4-(5-(4-bromo-3-methylphenyl)-1,2,4-oxadiazol-3-yl)benzyl-t-butoxycarbonylamino)propanoic acid t-butyl ester

The same method as that of Reference Example 19 was performed by using 3-(4-N-hydroxyamidinobenzyl-t-butoxycarbonylamino)propanoic acid t-butyl ester (500.0 mg) obtained in Reference Example 18 and 4-bromo-3-methyl benzoic acid (206.0 mg, manufactured by Wako Pure Chemical Industries, Ltd.), so that 2s80 mg of the titled compound was obtained.

¹H-NMR (CDCl₃): 8.08-8.13 (3H, m), 7.88 (1H, dd, J=3.0, 9.0), 7.72 (1H, d, J=6.0), 7.37 (2H, brs), 4.53 (2H, brs), 3.42-3.53 (2H, m), 2.52 (3H, s), 2.46-2.52 (2H, m), 1.48-1.56 (2H, m), 1.43 (18H, s)

### Example 109

### 3-(4-(5-(4-(2-acetylphenyl)-3-methylphenyl) 1,2,4-oxadiazol-3-yl)benzylamino)propanoic acid

### Process 1

### 3-(4-(5-(4-(2-acetylphenyl)-3-methylphenyl-1,2,4-oxadiazol-3-yl)benzyl-t-butoxycarbonylamino)propanoic acid t-butyl ester

To a 1,4-dioxane solution (2 mL) of the 3-(4-(5-(4-bromo-3-methylphenyl)-1,2,4-oxadiazol-3-yl)benzyl-t-butoxycarbonylamino)propanoic acid t-butyl ester (50 mg) obtained in Reference Example 70 and 2-acetylphenylboronic acid (27.0 mg, manufactured by Aldrich), trisbenzylideneacetone dipalladium (4.4 mg, manufactured by Aldrich), tri-t-butylphosphine tetraphenylborate (6.3 mg, manufactured by Kanto Chemical Co., Inc), and cesium carbonate (45.9 mg, manufactured by Kanto Chemical Co., Inc) were added. The resultant mixture was stirred for 12 hours at 90° C. After the stirring was ended, the reaction solution was filtered by using Celite, and the filtered solution was concentrated under a reduced pressure. For the obtained residue, flash chromatography (as an elution solution, 9:1 (v/v) of hexane/ethyl acetate was used) was performed, so that the titled compound (34.0 mg) was obtained. ¹H-NMR (CDCl₃): 8.12 (21, d, J=9), 8.13 (1H, s), 8.08 (1H, d, J=9), 7.78 (1H, dd, J=3.9), 7.57 (1H, dt, J=3.9), 7.49 (1H, dt, J=3.9), 7.30-7.45 (2H, m), 7.18-7.31 (2H, m), 4.53 (2H, brs), 3.44-3.54 (2H, m), 2.45-2.55 (2H, m), 2.21 (3H, s), 2.18 (3H, s), 1.53-1.56 (2H, m), 1.44 (18H, s)

### Process 2

### 3-(4-(5-(4-(2-acetylphenyl)-3-methylphenyl)-1,2,4-oxadiazol-3-yl)benzylamino)propanoic acid

To 3-(4-(5-(4-(2-methylphenyl)-3-methylphenyl)-1,2,4-oxadiazol-3-yl)benzyl-t-butoxycarbonylamino)propanoic acid t-butyl ester (54.5 mg) obtained in Process 1, 4N HCl dioxane solution (2 mL, manufactured by Kokusan Kagaku Company) was added. The resultant mixture was stirred overnight at room temperature. After the stirring was ended, a product was taken by filtration and rinsed with diethyl ether, so that the titled compound was obtained. ESI-MS: 456.1 (M+H), RTime 3.27 min.

According to the procedures of Example 109, the same method was performed except that any one of raw compounds listed in Table 76 was used instead of 2-acetylphenylboronic acid. As a result, the compounds listed in Table 76 were obtained.

TABLE 76

| | | | | LCMS (ESI+) | |
|---|---|---|---|---|---|
| Exp. | SM. | Reagent | Structure | MASS | RT |
| 110 | 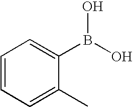 | Ald | 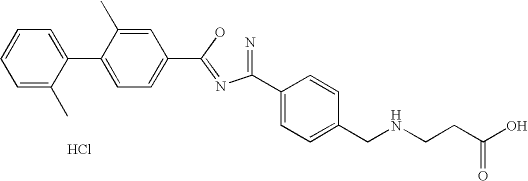 | 428.2 | 3.69 |
| 111 | 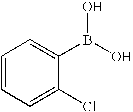 | Ald | 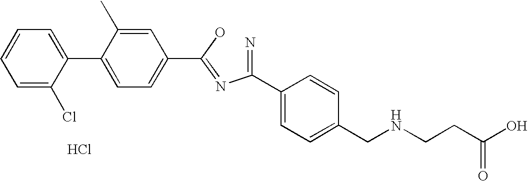 | 448.1 | 3.55 |
| 112 | 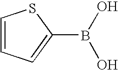 | LAN | 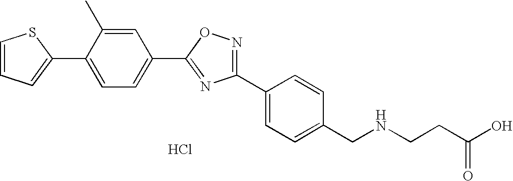 | 419.9 | 4.01 |
| 113 | 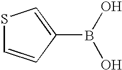 | WAKO | 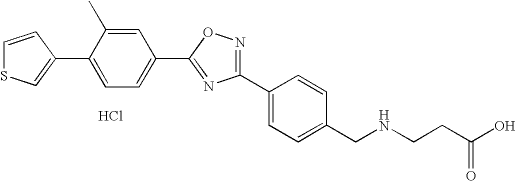 | 419.9 | 3.98 |
| 114 | 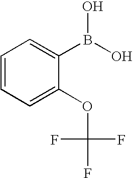 | WAKO | 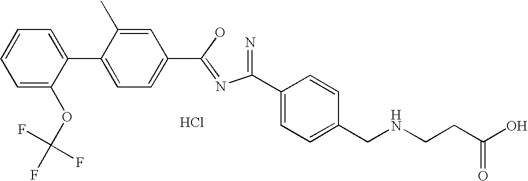 | 497.9 | 4.06 |
| 115 | 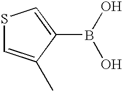 | Ald | 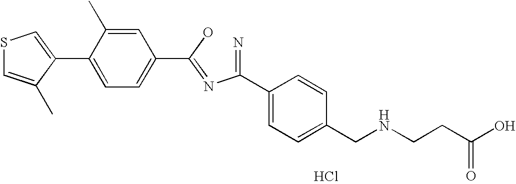 | 433.9 | 4.01 |
| 116 | 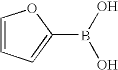 | Ald | 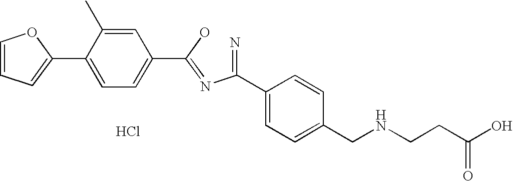 | 404.0 | 3.89 |
| 117 | 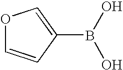 | Ald | 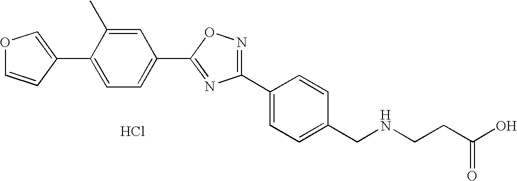 | 404.0 | 3.82 |
| 118 | 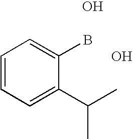 | Lan | 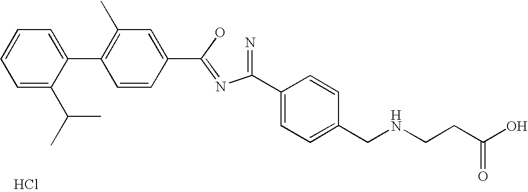 | 456.0 | 4.30 |

### Reference Example 71

### 3-(4-(5-(5-bromopyridin-2-yl)-1,2,4-oxadiazol-3-yl)benzyl-t-butoxycarbonylamino)propanoic acid t-butyl ester

According to the procedures of Example 70, the same method was performed except that 5-bromo picolinic acid (manufactured by Tokyo Chemical Industry Co., Ltd.) was used instead of 4-bromo-3-methylbenzoic acid. As a result, the titled compound was obtained.

¹H-NMR (CD₃OD): 8.95 (1H, s), 8.30 (2H, s), 8.14 (2H, d, J=8.1), 7.43 (2H, d, J=8.1), 4.56 (2H, s), 3.53 (2H, brs), 2.50 (2H, t, J=6.9), 1.44 (18H, s)

### Reference Example 72

### 3-(4-(5-(5-bromothiophen-2-yl)-1,2,4-oxadiazol-3-yl)benzyl-t-butoxycarbonylamino)propanoic acid t-butyl ester

According to the procedures of Reference Example 70, the same method was performed except that 5-bromo-2-thiophenecarboxylic acid (manufactured by Tokyo Chemical Industry Co., Ltd.) was used instead of 4-bromo-3-methylbenzoic acid. As a result, the titled compound was obtained. ¹H-NMR (CDCl₃): 8.08 (2H, d, J=8.1), 7.70 (1H, d, J=4.2), 7.60-7.63 (2H, m), 7.18 (1H, d, J=4.1), 4.52 (2H, brs), 3.42-3.54 (2H, m), 2.48-2.59 (2H, m), 1.43 (18H, s)

### Reference Example 73

### 3-(4-(5-(5-bromofuran-2-yl)-1,2,4-oxadiazol-3-yl)benzyl-t-butoxycarbonylamino)propanolc acid t-butyl ester

According to the procedures of Reference Example 70, the same method was performed except that 5-bromo-2-furancarboxylic acid (manufactured by Tokyo Chemical Industry Co., Ltd.) was used instead of 4-bromo-3-methylbenzoic acid. As a result, the titled compound was obtained. ¹H-NMR (CDCl₃): 8.10 (2H, d, J=8.4), 7.32-7.35 (2H, m), 7.32 (1H, d, J=3.6), 6.41 (1H, d, J=3.6), 4.51 (2H, brs), 3.42-3.65 (2H, m), 2.49 (2H, brs), 1.43 (18H, s)

Hereinafter, the compounds listed in Table 77 were synthesized in the same manner as in Example 109 except that the bromo compounds of Reference Examples 71 to 73 were used.

TABLE 77

| | | | | LCMS (ESI+) | |
|---|---|---|---|---|---|
| Exp. | SM. | Reagent | Structure | MASS | RT |
| 119 | 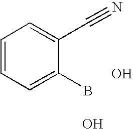 | Ald | 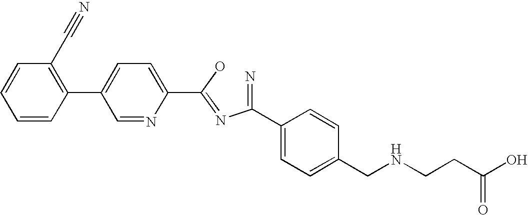 | 426.1 | 2.94 |
| 120 | 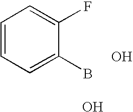 | Ald | 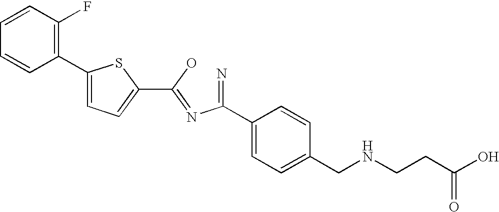 | 424.2 | 3.60 |
| 121 | 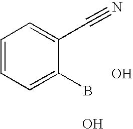 | Ald | 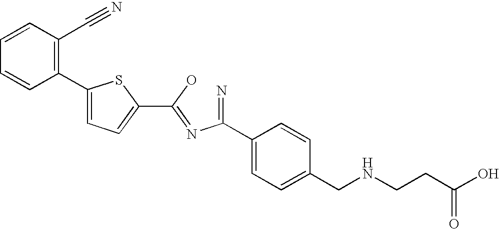 | 431.2 | 3.27 |
| 122 | 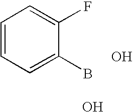 | Ald | 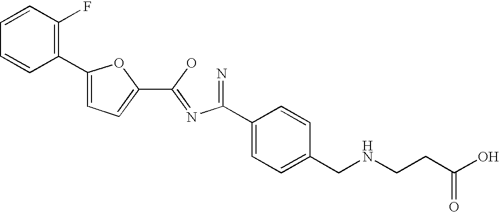 | 408.2 | 3.41 |

### Example 123

### 3-((5-(5-(4-isobutylphenyl)1,2,4-oxadiazol-3-yl)thiophen-2-yl)methylamino)propanoic acid

### Process 1

### 5-formylthiophene-2-carbonitrile

A tetrahydrofuran solution (250 mL) of lithium diisopropyl amide (1.09N hexane solution, 92.8 mL, manufactured by Kanto Chemical Co., Inc) was cooled down at −78° C., and a tetrahydrofuran solution (50 mL) of 2-cyanothiophene (8.55 mL, manufactured by Aldrich) was dropped thereto. After that, the resultant mixture was stirred for 45 minutes at the same temperature, N,N-dimethyl formamide (30 mL, manufactured by Kanto Chemical Co., Inc) was added to the mixture, and the resultant blend was stirred for 1 hour. After the stirring was ended, citric acid (20 g, manufactured by Wako Pure Chemical Industries, Ltd.) was added to the reaction solution and the resultant was poured into water. After tetrahydrofuran was distilled away, extraction was performed by using diethyl ether. An organic layer was washed with a saturated saline solution, and dried by using magnesium sulfate. After concentrating at a reduced pressure, chromatography (as an elution solution, 4:1 (v/v) of hexane/ethyl acetate was used) using Biotage 40M cartridge was performed, so that the titled compound (7.45 g) was obtained.

¹H-NMR (CDCl₃): 10.0 (1H, s), 7.77 (1H, d, J=3.3), 7.74 (1H, d, J-3.3)

### Process 2

### 3-((5-cyanothiophene-2-yl)methylamino)propanoic acid t-butyl ester

To a dichloromethane solution (150 mL) of 5-formylthiophen-2-carbonitrile (2.16 g) obtained in Process 1, β alanine-t-butyl ester hydrochloride (3.15 g, manufactured by Kokusan Kagaku Company) and sodium triacetoxyborohydride (6.7 g, manufactured by Aldrich) by using the same method as that of Reference Example 16 were added. The resultant mixture was stirred for 3 hours at room temperature. After the stirring was ended, the reaction solution was poured into a saturated sodium bicarbonate aqueous solution, and extraction was performed by using dichloromethane. An organic layer was washed with a saturated saline solution, dried by using magnesium sulfate, and further concentrated under a reduced pressure. For the residue, chromatography (as an elution solution, 4:1 (v/v) of hexane/ethyl acetate was used) using Biotage 40M cartridge was performed, so that the titled compound (2.94 g) was obtained.

### Process 3

### 3-((5-cyanothiophen-2-yl)methyl-t-butoxycarbonylamino) propanoic acid t-butyl ester

To a dichloromethane solution (100 mL) of 3-((5-cyanothiophen-2-yl)methylamino)propanoic acid t-butyl ester (2.94 g), obtained in Process 2, di-t-butylcarbonate (2.88 g, manufactured by Wako Pure Chemical Industries, Ltd.) and triethylamine (3.10 mL, manufactured by Wako Pure Chemical Industries, Ltd.) were added. The resultant mixture was stirred for 3 hours at room temperature. After the stirring was ended, the reaction solution was poured into water, and extraction was performed by using dichloromethane. An organic layer was washed with a saturated saline solution, dried by using magnesium sulfate, and concentrated under a reduced pressure. For the residue, chromatography (as an elution solution, 7:1 (v/v) of hexane/ethyl acetate was used) using Biotage 40M cartridge was performed, so that the titled compound (2.32 g) was obtained.

### Process 4

### N′-hydroxyamidino(5-(N′-hydroxycarbamiimide-yl) thiophen-2-yl)methyl-t-butoxycarbonylamino)propanoic acid t-butyl ester

According to the procedures of Reference Examples 18, to 3-((5-cyanothiophen-2-yl)methyl-t-butoxycarbonylamino) propanoic acid t-butyl ester (2.32 g) obtained in Process 3, a methanol solution (60 mL) of the compound, sodium bicarbonate (2.14 g, manufactured by Wako Pure Chemical Industries, Ltd.) and hydroxylamine hydrochloride (882.5 mg, manufactured by Kanto Chemical Co., Inc) were added. The resultant mixture was heated and refluxed for 4 hours.

After the stirring was ended, water was added to the reaction solution, and extraction was performed by using ethyl acetate. An organic layer was washed with a saturated saline solution, dried by using magnesium sulfate, and concentrated under a reduced pressure, so that the titled compound was obtained. The obtained residue was directly used for the next reaction. ¹H-NMR (CDCl₃): 7.10 (1H, d, J=9), 6.85 (1H, brs), 5.16 (2H, brs), 4.51 (2H, brs), 3.43 (2H, brs), 2.45 (2H, brs), 1.47 (9H, s), 1.42 (9H, s)

### Process 5

### 3-((5-(5-(4-isobutylphenyl)1,2,4-oxadiazol-3-yl) thiophene-2-yl)methyl-t-butoxycarbonylamino)propanoic acid t-butyl ester

49.1 mg of the titled compound was obtained in the same manner as in Reference Example 19 except that (5-(N′-hydroxycarbamiimide-yl)thiophen-2-yl)methyl-t-butoxy carbonylamino propanoic acid t-butyl ester (62.8 mg) obtained in Process 4 and 4-isobutylbenzoic acid (51.4 mg, manufactured by Tokyo Chemical Industry Co., Ltd.) were used.

¹H-NMR (CDCl₃); 7.89 (1H, s), 7.70 (1H, d, J=3.6), 7.46-7.48 (3H, m), 4.63 (2H, brs), 3.50 (2H, brs), 2.51 (2H, brs), 1.56 (3H, s), 1.51 (12H, s), 1.45 (12H, s)

### Process 6

### 3-((5-(5-(4-isobutylphenyl)1,2,4-oxadiazol-3-yl)thiophen-2-yl)methylamino)propanoic acid

To 3-((5-(5-(4-isobutylphenyl)1,2,4-oxadiazol-3-yl) thiophen-2-yl)methyl-t-butoxycarbonylamino)propanoic acid t-butyl ester (49.1 mg) obtained in Process 5, 4N HCl dioxane solution (2 mL, manufactured by Kokusan Kagaku Company) was added. The resultant mixture was stirred overnight at room temperature. After the stirring was ended, a product was taken by filtration, and dried under a reduced pressure, so that 30 mg of the titled compound was obtained. ESI-MS: 386.3 (M+H), RTime 4.00 min.

According to the procedures of Example 123, the same method was performed except that any one of raw compounds listed in Table 78 was used instead of 4-isobutylbenzoic acid. As a result, the compounds of Examples 124 to 128 listed in Table 78 were obtained.

TABLE 78

| | | | | | LCMS (ESI+) | |
|---|---|---|---|---|---|---|
| Exp. | Syn. | SM. | Reagent | Structure | MASS | RT |
| 124 | P2 | 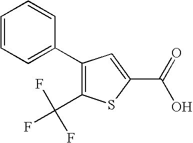 | MAY | 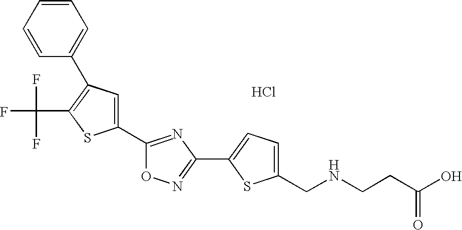 | 480.2 | 4.06 |
| 126 | P2 | 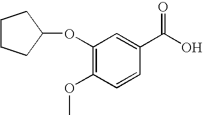 | MAY | 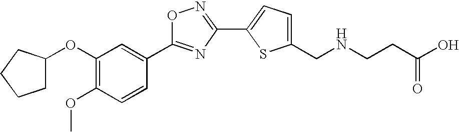 | 444.3 | 3.76 |
| 127 | P2 | 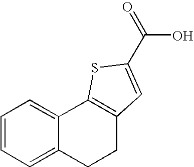 | Bionet | 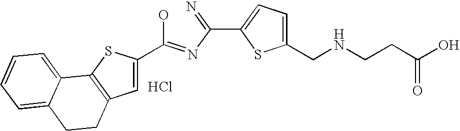 | 438.2 | 4.12 |
| 128 | P2 | 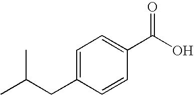 | TCI | 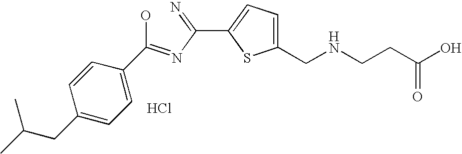 | 386.3 | 4.00 |

### Example 129

### 1,3-trans-3-(4-(5-(4-cyclohexylphenyl)-1,2,4-oxadiazol-3-yl)benzylamino)cyclobutanecarboxylic acid

### Process 1

### 4-(1,3-dioxolan-2-yl)benzonitrile

To a dichloromethane solution (100 mL) of 4-cyanobenzaldehyde (2.05 g, manufactured by Tokyo Chemical Industry Co., Ltd.), trimethyl orthoformate (2.05 mL, manufactured by Tokyo Chemical Industry Co., Ltd.), ethylene glycol (1.30 g, manufactured by Tokyo Chemical Industry Co., Ltd.), and p-toluenesulfonic acid monohydrate (296 mg, manufactured by Wako Pure Chemical Industries, Ltd.) were added. The resultant mixture was stirred for 3 hours at room temperature. After the stirring was ended, the reaction solution was poured into a saturated sodium bicarbonate aqueous solution, and extraction was performed by using dichloromethane. An organic layer was washed with a saturated saline solution, and dried by using magnesium sulfate. Subsequently, the organic layer was concentrated under a reduced pressure, so that the titled compound (2.50 g) was obtained.

### Process 2

### 4-(1,3-dioxolan)-N′-hydroxybenzamidine

The titled compound (1.75 g) was obtained in the same manner as in Reference Example 18 except that 4-(1,3-dioxolan-2-yl)benzonitrile (3.11 g) obtained in Process 1 was used.

### Process 3

### 5-(4-cyclohexylphenyl)-3-(4-(1,3-dioxolan-2-yl)phenyl)-1,2,4-oxadiazol

The same method as that of Reference Example 19 was performed except that 4-(1,3-dioxolan-2-yl)-N′-hydroxybenzamidine (200 mg) obtained in Process 2 and 4-cyclohexylbenzoic acid (234.9 mg, manufactured by Tokyo Chemical Industry Co., Ltd.) were used. As a result, the titled compound (191.6 mg) was obtained. ¹H-NMR (CDCl₃): 8.19 (2H, d, J-6.6), 8.13 (2H, d, J=6.6), 7.62 (2H, d, J=8.1), 7.38 (2H, d, J=8.1), 5.89 (1H, s), 4.07-4.18 (4H, m), 2.56-2.63 (1H, m), 1.56-1.90 (6H, m), 1.26-1.49 (6H, m)

### Process 4

### 4-(5-(4-cyclohexylphenyl)-1,2,4-oxadiazol-3-yl)benzaldehyde

To a tetrahydrofuran solution (9 mL) of 5-(4-cyclohexylphenyl)-3-(4-(1,3-dioxolan-2-yl)phenyl)-1,2,4-oxadiazole (191.6 mg) obtained in Process 3, a 5N HCl aqueous solution (1 mL, manufactured by Wako Pure Chemical Industries, Ltd.) was added. The resultant mixture was stirred for 5 hours at room temperature. After the stirring was ended, an organic layer was poured into a saturated saline solution, and extraction was performed by using ethylacetate. An organic layer was dried by using magnesium sulfate, and concentrated under a reduced pressure, so that the titled compound (162.7 mg) was obtained. ¹H-NMR (CDCl₃): 10.1 (1H, s), 8.36 (1H, d, J=8.1), 8.14 (1H, d, J=8.4), 8.03 (1H, d, J=8.4), 2.61-2.62 (1H, m), 1.56-1.90 (6H, m), 1.26-1.49 (6H, m)

The same method as that of Example 1 was performed except that

### 4-(5-(4-cyclohexylphenyl)-1,2,4-oxadiazol-3-yl)benzaldehyde (30 mg) obtained in Process 4 and

1,3-trans-aminocyclobutanecarboxylic acid hydrochloride were used. As a result, 3.9 mg of the titled compound was obtained. ESI-MS: 432.4 (M+H), RTime 4.12 min.

The following aldehydes of Examples 130 to 136 and Example 138 to 143 were obtained in the same manner as the procedures of Processes 1 to 4 of Example 129, except that the corresponding carboxylic acid was used instead of 4-cyclohexylbenzoic acid.

aldehyde 2

### 4-(5-(4-phenyl-5-(trifluoromethyl)thiophen-2-yl)-1,2,4-oxadiazol-3-yl)benzaldehyde

¹H-NMR (CDCl₃): 10.1 (1H, s), 8.33 (2H, d, J=8.4), 8.04 (2H, d, J=8.4), 7.94 (1H, s), 7.48-7.50 (5H, m)

aldehyde 3

### 4-(5-(4-isobutylphenyl-1,2,4-oxadiazol-3-yl)benzaldehyde

¹H-NMR (CDCl₃): 10.1 (1H, s), 8.36 (2H, d, J=8.1), 8.14 (2H, d, J=6.6), 8.03 (2H, d, J=6.6), 7.34 (2H, d, J=8.1), 2.59 (2H, d, J=7.2), 1.87-1.99 (1H, m), 0.94 (6H, d, J=7.2)

aldehyde 4

### 4-(5-(biphenyl-4-yl)-1,2,4-oxadiazol-3-yl)benzaldehyde ¹H-NMR (CDCl₃): 10.1 (1H, s), 8.38 (2H, d, J=8.1), 8.30 (2H, d, J=8.4), 8.04 (2H, d, J=8.4), 7.80 (2H, d, 8.1), 7.67 (2H, d, J=6.6), 7.40-7.53 (3H, m).

aldehyde 5

### 4-(5-(5-chloro-6-isobutoxypyridin-3-yl)-1,2,4-oxadiazol-3-yl)benzaldehyde

¹H-NMR (CDCl₃): 10.1 (1H, s), 8.88 (1H, d, J=1.8), 8.41 (1H, d, J=1.8), 8.34 (2H, d, J=8.4), 8.03 (2H, d, J=8.4), 4.27 (2H, d, J=6.6), 2.19 (1H, q, 6.6), 1.07 (6H, d, J=6.6) aldehyde 6

### 4-(5-(2-methylbiphenyl-4-yl)-1,2,4-oxadiazol-3-yl)benzaldehyde

¹H-NMR (CDCl₃): 10.1 (1H, s), 8.38 (2H, d, J=6.6), 8.14 (1H, s), 7.98-8.10 (3H, m), 7.31-7.50 (6H, m), 2.40 (3H, s)

aldehyde 7

### 4-(5-(4-isopropoxy-3-(trifluoromethyl)phenyl)-1,2,4-oxadiazol-3-yl)benzaldehyde

¹H-NMR (CDCl₃) δ 10.1 (1H, s), 8.44 (1H, s), 8.35 (3H, d, J=8.4), 8.03 (2H, d, J=8.4), 7.16 (2H, d, J=8.4), 4.97 (1H, dq, J=6.0), 1.45 (6H, d, J=6.0)

aldehyde 8

### 4-(5-(4-isobutoxy-3-(trifluoromethyl)phenyl)-1,2,4-oxadiazol-3-yl)benzaldehyde

¹H-NMR (CDCl₃); 10.1 (1H, s) 8.45 (1H, s), 8.33 (3H, d, J=8.7), 8.03 (2H, d, J=8.7), 7.14 (1H, d, J=8.7), 3.93 (2H, d, J=6.3), 2.20 (1H, q, J=6.3), 1.09 (6H, d, J=6.9)

aldehyde 9

### 4-(5-(4-pyridin-2-yl)phenyl)-1,2,4-oxadiazol-3-yl)benzaldehyde

¹H-NMR (CDCl₃): 10.1 (1H, s), 8.76 (1H, d, J=3.0), 8.38 (2H, d, J=8.1), 8.34 (2H, d, J=8.4), 8.22 (2H, d, J=8.4), 8.04 (2H, J=8.1), 7.82-7.84 (2H, m), 7.30-7.35 (1H, m).

According to the procedures of Example 129, the same method was performed except that any one of raw compounds listed in Table 79 was used instead of 4-(5-(4-cyclohexylphenyl)-1,2,4-oxadiazol-3-yl)benzaldehyde. As a result, the compounds of Examples 130 to 136 listed in Table 79 were obtained.

TABLE 79

| | | | | LCMS (ESI+) | |
|---|---|---|---|---|---|
| Exp. | Syn | Aldehyde | Structure | MASS | RT |
| 130 | A | 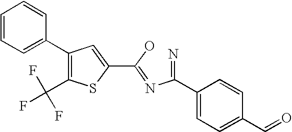 | 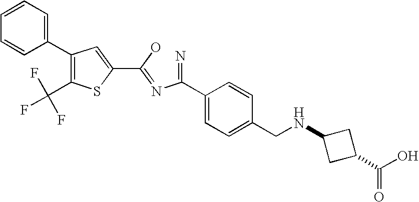 | 499.9 | 4.00 |
| 131 | A | 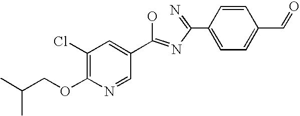 | 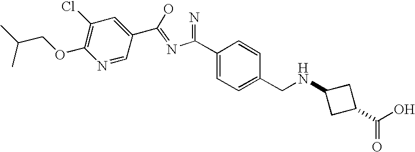 | 456.9 | 3.94 |
| 132 | A | 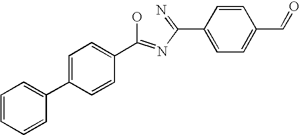 | 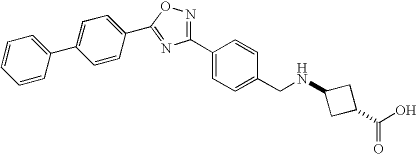 | 425.9 | 3.88 |
| 133 | A | 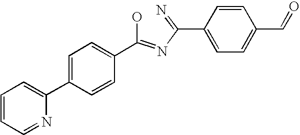 | 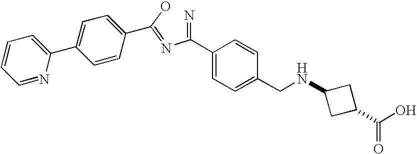 | 427.4 | 2.99 |
| 134 | A | 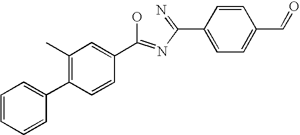 | 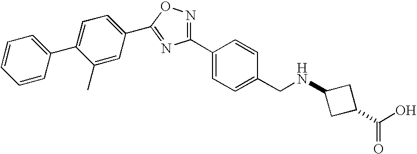 | 440.5 | 3.43 |
| 135 | A | 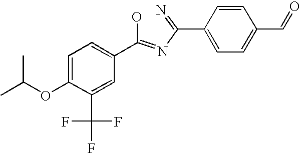 | 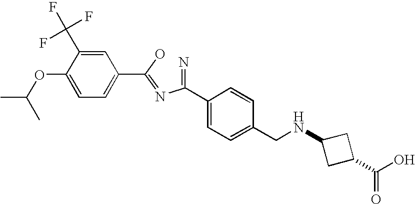 | 476.2 | 3.85 |
| 136 | A | 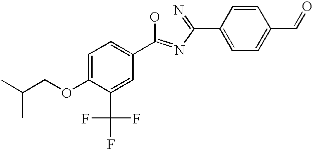 | 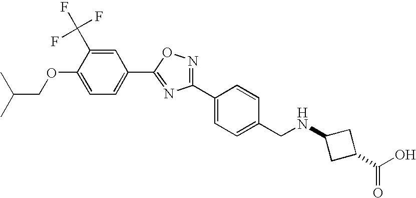 | 489.9 | 4.03 |

### Example 137

### (1s, 3R)-3-(4-(5-(4-cyclohexylphenyl)-1,2,4-oxadiazol-3-yl)benzylamino)cyclopentanecarboxylic acid

### Process 1

### (1s, 3s)-3-aminocyclopentanecarboxylic acid methyl ester hydrochloride

To (1s, 3s)—N-Boc-1-aminocyclopentane-3-carboxylic acid methyl ester (47.5 mg, manufactured by Acros Chemical Company), 4N HCl dioxane solution (2 mL, manufactured by Kokusan Kagaku Company) was added. The resultant mixture was stirred for 2 hours at room temperature. After the stirring was ended, the solvent was distilled away under a reduced pressure, so that the titled compound was obtained.

### Process 2

### (1s, 3R)-3-(4-(5-(4-cyclohexylphenyl)-1,2,4-oxadiazol-3-yl)benzylamino)cyclopentanecarboxylic acid methyl ester

To a dichloromethane solution (2 mL) of 4-(5-(4-cyclohexylphenyl)-1,2,4-oxadiazol-3-yl)benzaldehyde (43.2 mg) and (1s, 3s)-3-aminocyclopentanecarboxylic acid methyl ester hydrochloride, sodium triacetoxyborohydride (41.3 mg, manufactured by Aldrich) was added. The resultant mixture was stirred overnight at room temperature. After the stirring was ended, the reaction solution was concentrated. Subsequently, chromatography (as an elution solution 18:1 (v/v) of chloroform/methanol was used) using Biotage 12M cartridge was performed, so that 48.7 mg of the titled compound was obtained. ¹H-NMR (CDCl₃): 8.12 (2H, d, J=8.4), 8.12 (2H, d, J=8.1), 7.45 (2H, d, J=8.1), 7.38 (2H, d, J=8.4), 3.83 (2H, s), 3.67 (3H, s), 3.27-3.35 (1H, m), 2.94-3.05 (1H, m), 2.56-2.63 (1H, m), 1.73-2.13 (16H, m)

### Process 3

### (1s, 3R)-3-(4-(5-(4-cyclohexylphenyl)-1,2,4-oxadiazol-3-yl)benzylamino)cyclopentanecarboxylic acid

To a mixture solution of tetrahydrofuran and water at a ratio of 5:1 (6 mL) of the ester compound (48.7 mg) obtained in Process 2, lithium hydroxide monohydrate (4.5 mg, manufactured by Wako Pure Chemical Industries, Ltd.) was added. The resultant mixture was stirred overnight at 40° C. After the stirring was ended, the solvent was distilled away. Subsequently, chromatography (as an elution solution, 3:1:0.1 (v/v) of chloroform/methanol/ammonia aqueous solution was used) using Biotage 12M cartridge was performed, so that 39.7 mg of the titled compound was obtained. ESI-MS: 446.0 (M+H), RTime 4.09 min.

According to the procedures of Example 137, the same method was performed except that any one of raw compounds listed in Table 80 was used instead of 4-(5-(4-cyclohexylphenyl)-1,2,4-oxadiazol-3-yl)benzaldehyde. As a result, the compounds of Examples 138 to 143 listed in Table 80 were obtained.

TABLE 80

| | | | | LCMS (ESI+) | |
|---|---|---|---|---|---|
| Exp | Syn | Aldehyde | Structure | MASS | RT |
| 138 | A | 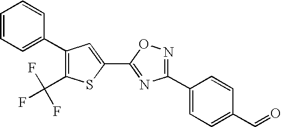 | 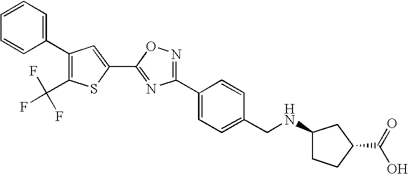 | 514.0 | 4.03 |
| 139 | A | 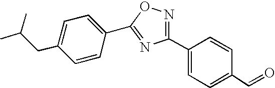 | 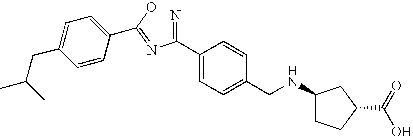 | 420.1 | 3.91 |
| 140 | A | 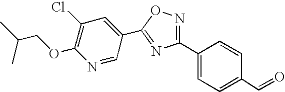 | 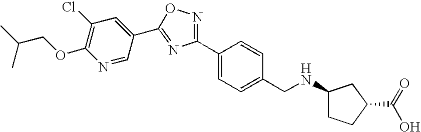 | 470.9 | 3.97 |
| 141 | A | 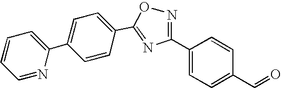 | 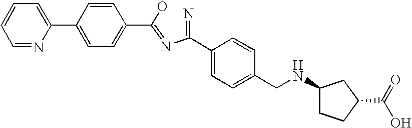 | 441.0 | 3.72 |
| 142 | A | 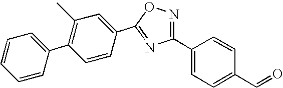 | 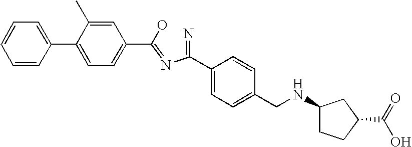 | 454.0 | 4.04 |
| 143 | A | 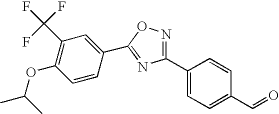 | 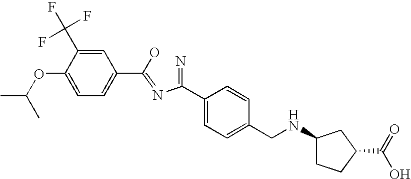 | 504.0 | 4.12 |

### Reference Example 74

### 4-formyl-2-methylbenzonitrile

n-Butyl lithium hexane solution (33.6 mL, manufactured by Kanto Chemical Co., Inc) was dripped to a tetrahydrofuran/hexane solution (246 mL/66 mL) of 4-bromo-2-methylbenzonitrile (9.45 g, manufactured by Lancaster) over 20 minutes at -95° C. or less at a nitrogen ambience. After the dripping was ended, the resultant mixture was stirred for 10 minutes at −95° C., and a tetrahydrofuran solution (52 mL) of N,N-dimethylformamide (4.48 mL, manufactured by Kanto Chemical Co., Inc) was dripped there into over 20 minutes at -95° C. or less. The resultant blend was further stirred for 10 minutes, and the temperature of the blend was increased to room temperature. A saturated ammonium chloride aqueous solution was added to the reaction solution, and extraction was performed by using ethyl acetate. An organic layer was washed with a saturated sodium bicarbonate solution and a saturated saline solution, dried by using sodium sulfate, and concentrated under a reduced pressure, so that 7.11 g of the titled compound was obtained. The obtained residue was directly used for the next reaction. ¹H-NMR (CDCl₃) 10.1 (1H, s), 7.83 (1H, s), 7.79 (2H, s), 2.65 (3H, s)

### Reference Example 75

### Process 1

### (1R,3R)-3-(t-butoxycarbonylamide)cyclobutanecarboxylic acid benzyl ester

To a dichloromethane solution (3.3 mL) of (1R,3R)-3-(t-butoxycarbonylamide)cyclobutanecarboxylic acid (215 mg, manufactured by AMRI), triethylamine (133 μL, manufactured by Wako Pure Chemical Industries, Ltd.) was added. The resultant mixture was cooled down to 0° C. After that, carbobenzoxy chloride (157 μL, manufactured by Wako Pure Chemical Industries, Ltd.) and N,N-dimethylaminopyridine (12.2 mg, manufactured by Wako Pure Chemical Industries, Ltd.) was added thereto, and the resultant blend was stirred for 1.5 hours. After the reaction was ended, 1N HCl aqueous solution was added to the reaction solution, and extraction was performed by using ethyl acetate. An organic layer was washed with a saturated sodium bicarbonate solution and a saturated saline solution, dried by using sodium sulfate, and concentrated under a reduced pressure. For the obtained residue, flash chromatography (as an elution solution, 5:1 (v/v) of hexane/ethyl acetate was used) was performed, so that 225 mg of the titled compound was obtained.

¹H-NMR (CDCl₃): 7.38-7.31 (5H, m), 5.14 (2H, s), 4.80-4.62 (1H, m)), 4.40-4.20 (1H, m), 3.04 (1H, ddd, J=1.1, J=4.0, J=9.7, J=13.2), 2.70-2.57 (2H, m), 2.26-2.12 (2H, m) 1.43 (9H, s)

### Process 2

### (1R,3R)-aminocyclobutanecarbonylbenzyl esterhydrochloride

To (1R,3R)-3-(t-Butoxycarbonylamide)cyclobutane carboxylic acid benzyl ester (225 mg) obtained in Process 1, a 4N1,4-dioxane solution (3.0 mL, manufactured by Kokusan Kagaku Company) was added. The resultant mixture was stirred for 1.5 hours. After the stirring was ended, diethyl ether was added thereto. A product was taken by filtration, and dried under a reduced pressure, so that 185 mg of the titled compound was obtained. ESI-MS: 206.1 (M+H), RTime 1.68 min.

The same methods as those of Reference Examples 16, 17, and 18 were used except that the raw material listed in Table 81 were used, so that the following group of compounds was synthesized.

TABLE 81

| Ref. | Syn. | SM. | Reagent | SM. | Reagent |
|---|---|---|---|---|---|
| 76 | A | 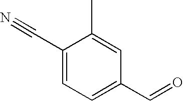 | Syn | 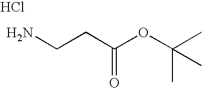 | KOK |
| 77 | A | 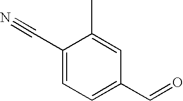 | Syn | 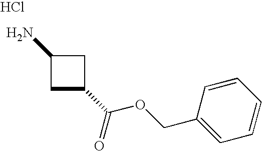 | Syn |

| | | LCMS (ESI+) | |
|---|---|---|---|
| Ref. | Structure | MASS | RT |
| 76 | 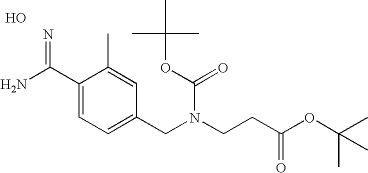 | 408.4 | 3.11 |
| 77 | 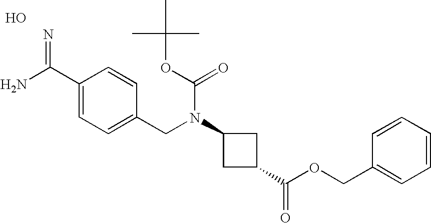 | 468.0 | 3.85 |

The compounds listed in Table 82 were synthesized. Here, as the raw materials, products listed in Table 81 were used. Moreover, in Examples 144 to 145, the same methods as those of Reference Example 19 and Example 38 were used. In Example 147, the same methods as those of Reference Example 19 and Example 146 were used.

### Example 146

### (1R,3R)-3-(4-(5-(4-isobutylphenyl)-1,2,4,-oxadiazol-3-yl)-3-methylbenzylamino)cyclobutanecarboxylic acid

To a methanol solution (1.0 mL) of (1R,3R)-3-(tert-butoxycarbonyl(4-(5-(4-isobutylphenyl)-1,2,4,-oxadiazol-3-yl)-3-methylbenzyl)amino)cyclobutane carboxylic acid benzyl ester (18.0 mg), a 5N sodium hydroxide aqueous solution (17.7 μL, manufactured by Wako Pure Chemical Industries, Ltd.) was added. After the resultant mixture was stirred for 16 hours, a 5N sodium hydroxide aqueous solution (20.0 μL, manufactured by Wako Pure Chemical Industries, Ltd.) was added thereto, and the resultant blend was stirred for 9 hours. After the stirring was ended, a 1N HCl aqueous solution (manufactured by Wako Pure Chemical Industries, Ltd.) was added thereto, and extraction was performed by using ethyl acetate. An organic layer was dried by using magnesium sulfate, and concentrated under a reduced pressure. A4NHCl dioxane solution (2 mL, manufactured by Kokusan Kagaku Company) was added to the obtained residue, and the resultant mixture was stirred overnight at room temperature. After the stirring was ended, a product was taken by filtration, and dried under a reduced pressure, so that 3.2 mg of the titled compound was obtained. ESI-MS: 420.6 (M+H), RTime 3.65 min.

TABLE 82

| | | | | | LCMS (ESI+) | |
|---|---|---|---|---|---|---|
| Exp. | Syn. | SM. | Reagent | Structure | MASS | RT |
| 144 | P2 | 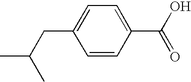 | TCI | 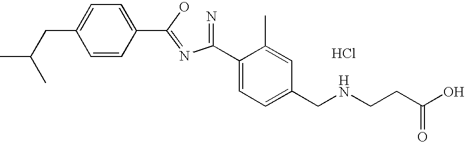 | 394.0 | 4.10 |
| 145 | P2 | 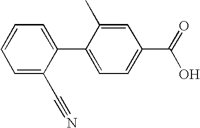 | Syn | 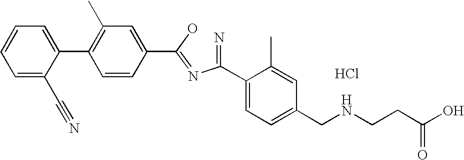 | 453.2 | 3.32 |
| 147 | P2 | 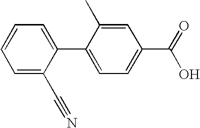 | Syn | 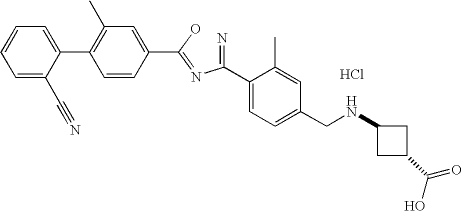 | 479.6 | 3.36 |

### Example 148

### 3-(3-methyl-4-(4-phenyl-5-(trifluoromethyl) thiophene-2-yl)methoxybenzylamino)propanoic acid

### Process 1

### 4-formyl-2-methylphenyl acetate

To a pyridine solution (10 mL) of 4-hydroxy-3-methylbenzaldehyde (1.026 g, manufactured by Aldrich), anhydrous acetic acid (10 mL, manufactured by Wako Pure Chemical Industries, Ltd.) was added. The resultant mixture was stirred for 4.5 hours at room temperature. After the stirring was ended, the reaction solution was concentrated, and dried under a reduced pressure, so that 1.405 g of the titled compound was obtained. The obtained residue was directly used for the next reaction. ESI-MS: 179.2 (M+H), RTime 4.15 min.

### Process 2

### 3-(3-methyl-4-acetoxybenzylamino)propanoic acid t-butyl ester

To a dichloromethane solution (20 mL) of 4-formyl-2-methylphenyl acetate (1.405 g) obtained in Process 1 and β-alanine t-butyl ester hydrochloride (1.504 g, manufactured by Kokusan Kagaku Company), sodium triacetoxyborohydride (3.341 g, manufactured by Aldrich) was added. The resultant mixture was stirred overnight at room temperature. After the stirring was ended, a saturated sodium bicarbonate aqueous solution was added to the reaction solution, and purification was performed by using ChemElute CE2050 (manufactured by Varian) and dichloromethane. Subsequently, an organic layer was concentrated under a reduced pressure, so that 2.62 g of the titled compound was obtained. The obtained residue was directly used for the next reaction. ESI-MS: 308.3 (M+H), RTime 3.37 min.

### Process 3

### 3-(3-methyl-4-acetoxybenzyl-t-butoxycarbonylamino)propanoic acid t-butyl ester

To a dichloromethane solution (20 mL) of 3-(3-methyl-4acetoxybenzylamino) propanoic acid t-butyl ester (2.62 g) obtained in Process 2, triethylamine (2.27 mL, manufactured by Wako Pure Chemical Industries, Ltd.) and di-t-butyl carbonate (2.791 g, manufactured by Wako Pure Chemical Industries, Ltd.) were added. The resultant mixture was stirred overnight at room temperature. After the stirring was ended, the reaction solution was concentrated under a reduced pressure. Subsequently, flash chromatography (as an elution solution, 24/1 (v/v) of hexane/ethyl acetate solution was used) was performed, so that 2.03 g of the titled compound was obtained. ESI-MS: 408.4 (M+H), RTime 5.47 min.

### Process 4

### 3-(3-methyl-4-hydroxybenzyl-t-butoxycarbonylamino)propanoic acid t-butyl ester

To a methanol solution (30 mL) of 3-(3-methyl-4-acetoxybenzyl-t-butoxycarbonylamino)propanoic acid t-butyl ester (1.905 g) obtained in Process 3, potassium carbonate (1.938 g, manufactured by Wako Pure Chemical Industries, Ltd.) was added. The resultant mixture was stirred for 3 hours at room temperature. After the stirring was ended, a saturated ammonium chloride aqueous solution was added to the reaction solution at 0° C., and purification was performed by using ChemElute CE2010 (manufactured by Varian) and dichloromethane. Subsequently, an organic layer was concentrated under a reduced pressure, so that 1.703 g of the titled compound was obtained. ESI-MS: 364.4 (M−H), RTime 5.07 min.

### Process 5

### 3-(3-methyl-4-(4-phenyl-5-(trifluoromethyl)thiophen-2-yl)methoxy benzyl-t-butoxycarbonylamino)propanoic acid t-butyl ester

To a tetrahydrofuran solution (3 mL) of 3-(3-methyl-4-hydroxybenzyl-t-butoxycarbonylamino)propanoic acid t-butyl ester (50 mg) obtained in Process 4, 2-hydroxymethyl-4-phenyl-5-trifluoromethyl-thiophene (106 mg), N,N,N′,N′-tetra methylazodicarboxamide (70.7 mg, manufactured by Midori Kagaku Co., Ltd.) and tributylphosphine (96 μL, manufactured by Kanto Chemical Co., Inc) were added. The resultant mixture was stirred for 16 hours at room temperature. After the stirring was ended, the reaction solution was filtered, and the filtered solution was concentrated under a reduced pressure. For the obtained residue, flash chromatography (as an elution solution, 15:1 (v/v) of hexane/ethylacetate was used) was performed, so that 61.3 mg of the titled compound was obtained.

¹H-NMR (CDCl₃): 7.43-7.39 (5H, m), 7.06 (3H, s), 6.83 (1H, d, J=6.0), 5.22 (2H, s), 4.37 (2H, s), 3.55-3.30 (2H, m), 2.45 (2H, s), 2.27 (3H, s), 1.48 (9H, s), 1.43 (9H, s)

The aforementioned 2-hydroxymethyl-4-phenyl-5-trifluoromethyl-thiophene was synthesized based on a method disclosed in the document of J, Med. Chem. vol. 47, p. 6662 (2004).

### Process 6

### 3-(3-methyl-4-(4-phenyl-5-(trifluoromethyl)thiophene-2-yl)methoxybenzylamino)propanoic acid

To 3-(3-methyl-4-(4-phenyl-5-(trifluoromethyl)thiophene-2-yl)methoxybenzyl-t-butoxycarbonylamino)propanoic acid t-butyl ester (58.7 mg) obtained in Process 5, a 4N HCl dioxane solution (2 mL, manufactured by Kokusan Kagaku Company) was added. The resultant mixture was stirred overnight at room temperature. After the stirring was ended, a product was taken by filtration and rinsed with diethyl ether, so that the titled compound was obtained. ESI-MS: 450.1 (M+H), RTime 4.03 min

The same method as that of Example 148 was performed except that the raw materials listed in Table were used instead of 4-hydroxy-3-methylbenzaldehyde. As a result, the compounds of Examples 149 to 152 listed in Table 83 were obtained.

TABLE 83

| | | | | | LCMS (ESI+) | |
|---|---|---|---|---|---|---|
| Exp. | Syn. | SM. | Reagent | Structure | MASS | RT |
| 149 | A | 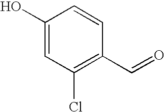 | Ald | 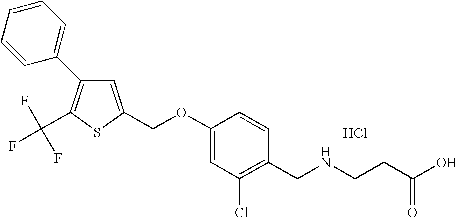 | 470.1 | 4.00 |
| 150 | A | 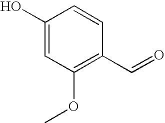 | Fluka | 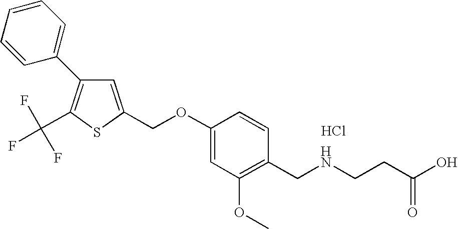 | 466.2 | 3.94 |
| 151 | A | 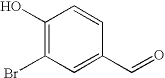 | Ald | 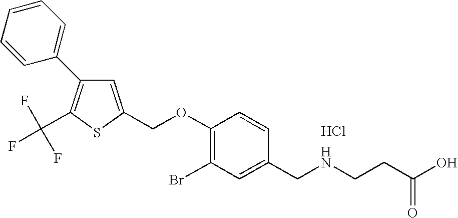 | 514.1 | 4.00 |
| 152 | A | 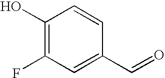 | Matrix | 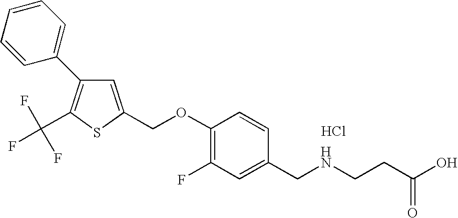 | 454.2 | 3.97 |

### Reference Example 78

### 3-methyl-4-(4,4,5,5-tetra methyl-1,3,2-dioxaborolan-2-yl)benzoic acid methyl ester

To a dioxane solution (8 mL) of 4-bromo-3-methylbenzoic acid methyl ester (1.0 g, manufactured by Tokyo Chemical Industry Co., Ltd.) and bis(pinacolato)diboran (1.22 g, manufactured by Aldrich), potassium acetate (857.7 mg, manufactured by Kanto Chemical Co., Inc), [1,11-bis(diphenylphosphino)ferrocene] dichloropalladium, dichloromethane complex (1:1) (178.4 mg, manufactured by Aldrich) were added. The resultant mixture was stirred for 3 hours at 100° C. After the stirring was ended, the reaction solution was filtered by using Celite, and concentrated under a reduced pressure. For the obtained residue, flash chromatography (as an elution solution, 15:1 (v/v) of hexane/ethyl acetate was used) was performed, so that 1.16 g of the titled compound was obtained.

¹H-NMR (CDCl₃): 7.85 (1H, s), 7.82 (2H, s), 4.15 (3H, s), 2.57 (3H, s), 1.35 (12H, s).

### Reference Example 79

### 3-methyl-4-(3-trifluoromethylpyridin-2-yl)benzoic acid methyl ester

A 1,2-dimethoxyethane solution (5 mL) of 3-methyl-4-(4,4,5,5-tetra methyl-1,3,2-dioxaborolan-2-yl)benzoic acid methyl ester (158 mg) obtained in Reference Example 78 and 2-bromo-3-trifluoromethylpyridine (122.0 mg, manufactured by Matrix), tetrakis triphenyl phosphine palladium (31.2 mg, manufactured by Kanto Chemical Co., Inc) and a 1M potassium carbonate aqueous solution (1 mL) were added. The resultant mixture was heated and refluxed for 4 hours. After the reaction was ended, the reaction solution was poured into saturated sodium bicarbonate, and extraction was performed by using ethyl acetate. An organic layer was dried by using sodium sulfate, and concentrated under a reduced pressure. For the obtained residue, flash chromatography (as an elution solution, 4:1 (v/v) of hexane/ethyl acetate was used) was performed, so that 119.4 g of the titled compound was obtained.

¹H-NMR (CDCl₃): 8.86 (1H, d, J=6.0), 8.11 (1H, d, J=6.0), 7.98 (1H, s), 7.98 (1H, d, J=6.0), 7.46-7.50 (1H, m), 7.25-7.28 (1H, m), 3.94 (3H, s), 2.01 (3H, s)

### Reference Example 80

### 3-methyl-4-(3-trifluoromethylpyridin-2-yl)benzoic acid

To an ethanol solution (5 mL) of 3-methyl-4-(3-trifluoromethylpyridin-2-yl)benzoic acid methylester (119 mg) obtained in Reference Example 79, a 5N sodium hydroxide aqueous solution (1 mL, manufactured by Wako Pure Chemical Industries, Ltd.) was added. The resultant mixture was stirred for 3 hours at room temperature. After the stirring was ended, a 5N HCl aqueous solution (1 mL, manufactured by Wako Pure Chemical Industries, Ltd.) was added to the reaction solution, and the resultant blend was poured into a saturated saline solution. The mixture solution was extracted by using ethyl acetate. An organic layer was dried by using sodium sulfate, and concentrated under a reduced pressure, so that the titled compound (110 mg) was obtained. ESI-MS: 282.2 (M+H), RTime 1.57 min.

### Example 153

### 3-(4-(5-(3-methyl-4-(3-(trifluoromethyl)pyridin-2-yl)phenyl)-1,2,4-oxadiazol-3-yl)benzylamino)propanoic acid

The same methods as those of Reference Example 19 and Example 38 were performed except that 3-methyl-4-(3-trifluoromethylpyridin-2-yl)benzoic acid obtained in Reference Example 80 was used. As a result, the titled compound was obtained.

### ESI-MS: 483.1 (M+H), RTime 1.24 min.

Carboxylic acids listed in Table 84 were synthesized in the same manner as the procedures of Reference Example 28, except that any one of raw compounds listed in Table 84 was used.

TABLE 84

| | | | | | | | LCMS (ESI−) | |
|---|---|---|---|---|---|---|---|---|
| Exp. | Syn. | SM. | Reagent | SM. | Reagent | Structure | MASS | RT |
| 81 | X | 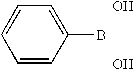 | TCI | 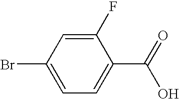 | TCI | 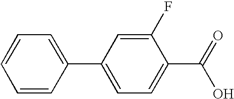 | 215.1 | 3.90 |
| 82 | X | 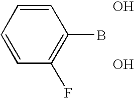 | WAKO | 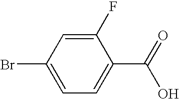 | TCI | 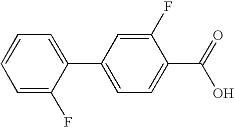 | 233.1 | 3.90 |

According to the procedures of Reference Example 19 and Example 38, the same method was performed except that any one of raw compounds listed in Table 85 was used instead of 4-cyclohexylbenzoic acid. As a result, the compounds of Examples 154 to 160 listed in Table 85 were obtained.

TABLE 85

| | | | | | LCMS (ESI+) | |
|---|---|---|---|---|---|---|
| Exp. | Syn. | SM. | Reagent | Structure | MASS | RT |
| 154 | P2 | 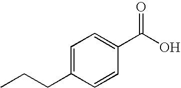 | TCI | 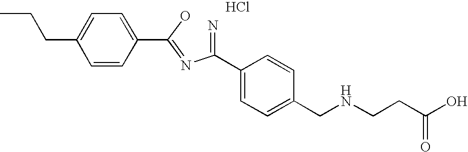 | 366.3 | 1.37 |
| 155 | P2 | 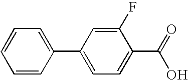 | Syn | 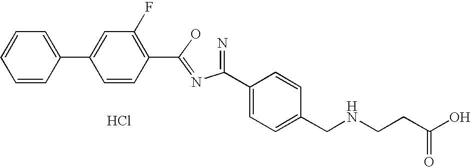 | 418.3 | 1.35 |
| 156 | P2 | 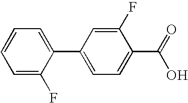 | Syn | 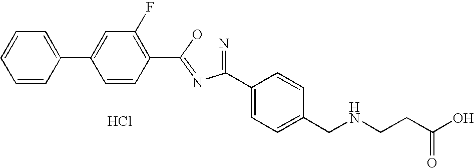 | 436.1 | 1.37 |
| 157 | P2 | 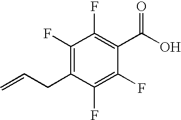 | ALD | 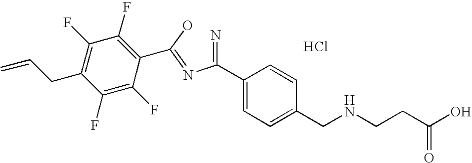 | 436.3 | 1.37 |
| 158 | P2 | 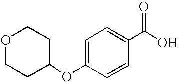 | MAY | 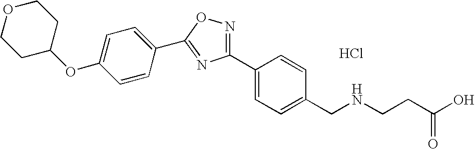 | 424.3 | 1.07 |
| 159 | P2 | 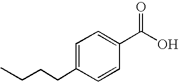 | TCI | 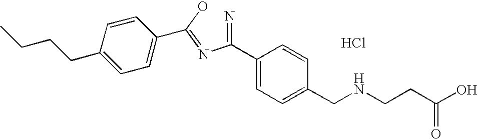 | 380.3 | 1.45 |
| 160 | P2 | 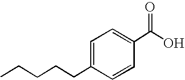 | TCI | 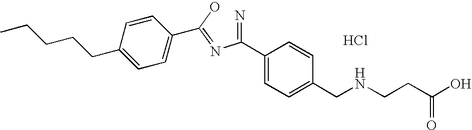 | 394.3 | 1.56 |

### Test Example 11

### ³⁵s-GTPγS binding assay using membrane preparation of CHO cell stably expressing S1P1 receptor

A G protein-GTPγS binding test associated with a ligand was performed by using a membrane preparation which was obtained from a CHO cell which stably overexpressed S1P1 receptor. In a 96 well-micro-titer dish, the produced membrane protein and a compound which was diluted with a solvent such as DMSO or sphingosine-1-phosphate in various concentrations was incubated in a solution containing 20 mM of Tris-Cl (pH7.5), 100 mM of NaCl, 10 mM of MgCl₂, 5μM of GDP, 0.1% of BSA, and 125 μM of ³⁵s-GTPγS (specific radioactivity, 1250 Ci mmol). The binding was performed for 1 or more hours at room temperature, and the membrane was harvested on a GF/B filter plate by using a Millipore's MultiScreen Separation System to end the binding. After the filter plate was dried for 30 or more minutes, 25 μL of MicroScint-20 was added to each well, and the binding was measured by using the TopCount.

For the agonist activity of compound, the value of a well which a solvent was added to was set to a control value and compared with an increment of an evaluation compound-added well, so that the increment of each concentration of the compound was obtained. EC50 value was calculated under the definition that the value is an agonist concentration required to provide 50% of the maximum increment thereof.

The results are listed in Table 86.

In addition, a ratio (S1P1/S1P3) to the EC50 value for the S1P3 receptor described in Test Example 2 can also be calculated. In addition, similarly, a ratio (S1P1/S1P2) to the EC50 value for the S1P2 receptor described in Test Example 3, a ratio (S1P1/S1P4) to the EC50 value for the S1P4 receptor described in Test Example 4, and a ratio (S1P1/S1P5) to the EC50 value for the S1P5 receptor described in Test Example 5 may be calculated. An effectiveness of an active ingredient of a pharmaceutical product can be verified.

TABLE 86

| ex. No. | EC₅₀ (nM) |
|---|---|
| 4 | 34 |
| 9 | 161 |
| 13 | 234 |
| 15 | 121 |
| 16 | 94 |
| 18 | 251 |
| 22 | 268 |
| 24 | 87 |
| 25 | 4 |
| 27 | 370 |
| 35 | 160 |
| 37 | 64 |
| 40 | 0.5 |
| 41 | 25 |
| 43 | 46 |
| 44 | 13 |
| 45 | 1.6 |
| 46 | 0.5 |
| 48 | 3 |
| 49 | 0.6 |
| 50 | 42 |
| 54 | 0.34 |
| 55 | 34 |
| 57 | 69 |
| 58 | 56 |
| 59 | 11 |
| 61 | 1.5 |
| 62 | 0.4 |
| 67 | 0.8 |
| 68 | 5.5 |
| 69 | 14 |
| 73 | 0.07 |
| 74 | 0.12 |
| 75 | 0.34 |
| 76 | 0.35 |
| 77 | 13 |
| 78 | 0.26 |
| 82 | 1.8 |
| 83 | 11 |
| 86 | 0.31 |
| 88 | 10 |
| 89 | 0.17 |
| 90 | 1.4 |
| 91 | 0.25 |
| 92 | 0.2 |
| 93 | 0.48 |
| 94 | 0.25 |
| 95 | 25 |
| 98 | 0.37 |
| 99 | 1.3 |
| 100 | 1.7 |
| 101 | 0.41 |
| 102 | 0.41 |
| 103 | 3.9 |
| 104 | 2.1 |
| 105 | 0.49 |
| 106 | 1.2 |
| 108 | 0.29 |
| 110 | 0.35 |
| 111 | 0.17 |
| 112 | 0.34 |
| 113 | 0.07 |
| 114 | 0.19 |
| 115 | 0.04 |
| 116 | 1.5 |
| 117 | 0.07 |
| 118 | 0.45 |
| 120 | 3.6 |
| 124 | 0.2 |
| 128 | 16 |
| 130 | 4.5 |
| 134 | 1.2 |
| 135 | 0.72 |
| 136 | 4 |
| 138 | 21 |
| 139 | 23 |
| 142 | 6.7 |
| 144 | 0.27 |
| 145 | 0.2 |
| 146 | 4.5 |
| 147 | 2.4 |
| 149 | 4.5 |
| 150 | 37 |
| 151 | 27 |
| 152 | 6.4 |

### Test Example 2

### ³⁵s-GTPγS binding assay using membrane preparation of CHO cell of temporarily expressing S1P3 receptor

A GTPγS binding test associated with S1P3 receptor was performed by using the same method as that of the GTPγS binding test associated with S1P1 receptor. In the test, a membrane protein which was produced from a CHO cell in which S1P3 gene was temporarily transfected was used.

For the agonist activity of compound, the value of a well which a solvent was added to was set to a control value and compared with an increment of an evaluation compound-added well, so that the increment of each concentration of the compound was obtained. EC50 value was calculated under the definition that the value is an agonist concentration required to provide 50% of the highest increment thereof. The results are listed in Table 87.

TABLE 87

| ex. No. | EC₅₀ (nM) |
|---|---|
| 4 | 870 |
| 25 | 117 |

### Test Example 3

### ³⁵s-GTPγS binding assay using membrane preparation of cell (S1P2 expressing cell) overexpressing S1P2 receptor

A GTPγS binding test associated with S1P2 receptor may be performed by using the same method as that of the GTPγS binding test associated with S1P1 receptor. In the test, a membrane protein which is produced from a CHO cell which temporarily or stably overexpresses S1P2 receptor is used.

For the agonist activity of compound, the value of a well which a solvent is added to is set to a control value and compared with an increment of an evaluation compound-added well, so that the increment of each concentration of the compound is obtained. EC50 value is calculated under the definition that the value is an agonist concentration required to provide 50% of the highest increment thereof.

### Test Example 4

### ³⁵s-GTPγS Binding Assay Using Membrane Preparation of Cell (S1P4 Expressing Cell) Overexpressing S1P4 Receptor

A GTPγS binding test associated with S1P4 receptor may be performed by using the same method as that of the GTPγS binding test associated with S1P1 receptor. In the test, a membrane protein which is produced from a CHO cell which temporarily or stably overexpresses S1P4 receptor is used.

For the agonist activity of compound, the value of a well which a solvent is added to is set to a control value and compared with an increment of an evaluation compound-added well, so that the increment of each concentration of the compound is obtained. EC50 value is calculated under the definition that the value is an agonist concentration required to provide 50% of the highest increment thereof.

### Test Example 5

### ³⁵s-GTPγS Binding Assay Using Membrane Preparation of Cell (SIPS Expressing Cell) Overexpressing S1P5 Receptor

A GTPγS binding test associated with S1P5 receptor may be performed by using the same method as that of the GTPγS binding test associated with S1P1 receptor. In the test, a membrane protein which is produced from a CHO cell which temporarily or stably overexpresses S1P1 receptor is used.

For the agonist activity of compound, the value of a well which a solvent is added to is set to a control value and compared with an increment of an evaluation compound-added well, so that the increment of each concentration of the compound is obtained. EC50 value is calculated under the definition that the value is an agonist concentration required to provide 50% of the highest increment thereof.

### Test Example 6

### S1P1 Receptor-Ligand Binding Assay

The activity of compound to S1P1 can be evaluated by using the following method instead of the method of Test Example 1. Similar to Test Example 1, a membrane protein which is produced from a CHO cell in which S1P1 gene is temporarily transfected or a CHO cell which stably overexpresses the receptor is used.

In a 96 well-micro-titer dish, the produced membrane protein and a compound which is diluted with a solvent such as DMSO or ³³P sphingosine-1-phosphate in various concentrations is incubated in a solution containing 20 mM of Tris-Cl (pH7.5), 100 mM of NaCl, 15 mM of NaF, 2 mM of Deoxypyridoxine, and 4 mg/mL of BSA. The binding is performed for 1 hour at 30° C., and the membrane is harvested on a GF/C filter plate by using a Millipore's MultiScreen Separation System. After the filter plate is dried for 30 or more minutes, 25 μL of MicroScint-20 is added to each well, and radioactivity is measured by using a top count method. Non-specific binding is defined with an amount of radioactivity which remains under the presence of 1 μm or more of non-radioactive sphingosine-1-phosphate. For the antagonist activity of compound, the value of a well which a solvent is added to is set to the maximum binding value and compared with a non-specific binding value, so that the binding inhibition rate of each concentration of the compound is obtained. IC₅₀ value is calculated under the definition that the value is an antagonist concentration required to inhibit 50% of the binding.

In addition, a ratio (S1P1/S1P3) to the IC₅₀ value for the S1P3 receptor described in Test Example 7 can also be calculated. In addition, similarly, a ratio (S1P1/S1P2) to the IC₅₀ value for the S1P2 receptor described in Test Example 8, a ratio (S1P1/S1P4) to the EC50 value for the S1P4 receptor described in Test Example 9, and a ratio (S1P1/S1P5) to the IC₅₀ value for the S1P5 receptor described in Test Example 10 may be calculated. In addition, the agonist and antagonist effects to the S1P1 receptor can be evaluated by comparing with the results of the ³⁵s-GTPγS binding assay described in Test Example 1.

### Test Example 7

### S1P3 Receptor-Ligand Binding Assay

The activity of compound to S1P3 receptor can be evaluated by using a ligand binding assay.

The S1P3 receptor-ligand binding assay can be performed by using the same method as that of the S1P1 receptor-ligand binding assay. Similarly to Test Example 2, a membrane protein which is produced from a CHO cell in which S1P3 gene is temporarily transfected or a CHO cell which stably overexpresses the receptor is used.

In addition, the agonist and antagonist effects to the S1P3 receptor can be evaluated by comparing with the results of the ³⁵s-GTPγS binding assay described in Test Example 2.

### Test Example 8

### S1P2 Receptor-Ligand Binding Assay

The activity of compound to S1P2 receptor can be evaluated by using a ligand binding assay. The S1P2 receptor-ligand binding assay can be performed by using the same method as that of the S1P1 receptor-ligand binding assay. Similarly to Test Example 3, a membrane protein which is produced from an S1P2-expressing cell is used.

In addition, the agonist and antagonist effects to the S1P2 receptor can be evaluated by comparing with the results of the ³⁵s-GTPγS binding assay described in Test Example 3.

### Test Example 9

### S1P4 Receptor-Ligand Binding Assay

The activity of compound to S1P4 receptor can be evaluated by using a ligand binding assay. The S1P4 receptor-ligand binding assay may be performed by using the same method as that of the S1P1 receptor-ligand binding assay. Similarly to Test Example ⁴, a membrane protein which is produced from an S1P4-expressing cell is used.

In addition, the agonist and antagonist effects to the S1P4 receptor can be evaluated by comparing with the results of the ³⁵s-GTPγS binding assay described in Test Example 4.

### Test Example 10

### S1P5 Receptor-Ligand Binding Assay

The activity of compound to S1P5 receptor can be evaluated by using a ligand binding assay. The S1P5 receptor-ligand binding assay may be performed by using the same method as that of the S1P1 receptor-ligand binding assay. Similarly to Test Example 5, a membrane protein which is produced from an S1P5-expressing cell is used.

In addition, the agonist and antagonist effects to the S1P5 receptor can be evaluated by comparing with the results of the ³⁵s-GTPγS binding assay described in Test Example 5.

### Test Example 11

Evaluation of Reduction in Peripheral Blood Lymphocyte count

A compound or a solvent was orally administered to a rat. When 3, 6, 24, 48, and 72 hours had elapsed since the administration of compound, blood was withdrawn from its tail vein. All the blood samples were analyzed hematologically. The total count of peripheral lymphocytes was measured by using an auto-analyzer (Sysmex 2000Xi). The effect of each compound to the total count of peripheral lymphocytes was evaluated by using three or more rats in one group. The reduction in lymphocyte count caused by the administration of compound was compared with that caused by the administration of solvent to a rat group. Namely, the average lymphocyte count of a solvent-administered group was set to 100%, and a control value (%) was calculated from the average lymphocyte count of a compound-administered group. An ED50 value, that is, a dosed amount of the compound required to reduce the lymphocyte count by 50% in three hours from the administration was calculated from the dosed amount of the compound and the control value (%). The results for the typical compounds according to the present invention are listed in Table 88.

TABLE 88

| ex. No. | ED₅₀ (mg/kg) |
|---|---|
| 6 | 5.1 |
| 40 | 0.71 |
| 46 | 2.1 |
| 54 | 0.44 |
| 61 | 0.53 |
| 73 | 0.065 |
| 78 | 0.37 |
| 90 | 4.1 |
| 93 | 0.64 |
| 94 | 0.087 |
| 104 | 0.70 |
| 105 | 0.084 |
| 106 | 0.50 |
| 117 | 0.88 |
| 135 | 0.16 |
| 145 | 0.46 |
| 147 | 0.72 |

### Test Example 12

### Evaluation of Effect to Heart

The effect of compound to heart function is monitored by using an electrocardiogram measuring apparatus (Power Lab 4/25T). The electrocardiogram before and after administration of compound to an anesthetized rat, mouse, or guinea pig is recorded to measure the heart rate.

After a compound solution is injected into a vein, a change in heart rate is measured over a period of 30 minutes or more from the injection. The effect of compound to heat rate is evaluated by using three or more tested objects in one group. The change in heart rate due to the administration of compound is obtained by comparing the heart rate after the compound administration with the heart rate of a solvent-administered group or the heart rate before the administration.

### Test Example 13

### Rat DTH Model

Hair on the abdomen of a Lewis female rat is removed with electric shaver. Sensitization is conducted by continuously applying 1% of dinitrofluorobenzene (DNFB) solution (100 μl) onto the abdomen for two days. After 5 days has elapsed from a starting date of the sensitization, induction is conducted by applying 0.5% of DNFB solution (20 μl) onto rat's auricle (right rear portion). A compound which is suspended in a 1% methylcellulose solution is forcibly oral-administered to the stomach by using an oral sonde one-time everyday for 6 days from the starting date of the sensitization. When 24 and 48 hours have elapsed since the DNFB application, a thickness of auricle of rat is measured by using thickness gauge (manufactured by Mitutoyo Corporation). The edema of the auricle is evaluated based on the thickness of auricle.

### Test Example 14

### Adjuvant-Induced Arthritis Model

7-weeks old Lewis female rats are used for evaluation. The volume of a hind paw of a rat was measured. As an adjuvant, 500 μg/100 μL of M. tuberclulosis H37 RA (manufactured by Difco) is subcutaneously injected into the plantar of the left hind paw, so that an adjuvant arthritis rat is produced. A compound which is suspended in a 1% methylcellulose solution is forcibly oral-administered to the stomach by using an oral sonde one-time everyday for 21 days from the injection date of the adjuvant. The evaluation of arthritis is performed by measuring the volume of paw of each tested subject by using PLETHYSMOMETER (manufactured by UGO BASILE) and comparing the measured deta of a compound-administered group with those of a solvent-administered group. Swelling of the plantar portion of hind leg of the solvent-administered group was set to 100%, and a control value (%) was calculated from the swelling of the compound-administered group. An ED50 value, that is, a dosed amount of the compound required to suppress the swelling of the plantar portion of hind paw on the 21-th day from the administration date of the adjuvant by 50% was calculated from the dosed amount of the compound and the control value (%). The results for the representative compounds according to the present invention are listed in Table 89.

TABLE 89

| ex. No. | ED₅₀ (mg/kg) |
|---|---|
| 54 | 0.33 |
| 135 | 0.21 |

### Test Example 151

### Collagen-Induced Arthritis Model

7-weeks old female DBA1J mice are used. A chicken cartilage II-type collagen solution (1% solution, Nippon Meat Packers Inc., 300-31601) and a Freund's complete adjuvant (231131, manufactured by DIFCO) are mixed to produce an emulsion. 100 μL of the emulsion (including 100 μg of collagen) is injected into skin of a tail-root portion of the mouse. In addition, as an additional sensitization, after three weeks, 100 μL of emulsion produced according to the aforementioned method is injected again into the skin of the tail-root portion, so that arthritis is induced. A compound which is suspended in a 1% methylcellulose solution is forcibly oral-administered to the stomach by using an oral sonde one-time or more times everyday after the date of firstly injecting the collagen or the date of the additional sensitization. Until the date of finally evaluating the arthritis, the administration is repeated.

The evaluation of the arthritis is performed by scoring a degree of arthritis with the maximum value of 5 for each leg and comparing the score of a compound-administered group with that of a solvent-administered group. Thus, the effect of the compound is measured.

### INDUSTRIAL APPLICABILITY

A compound of the present invention, a possible stereoisomer or racemic body thereof, or a pharmacologically acceptable salt, hydrate, or solvate thereof, or a prodrug thereof has an effect as an S1P1/Edg1 receptor agonist, so that it can be a useful active ingredient of a pharmaceutical product representing immune suppression activity and used in the associated pharmaceutical product industries.

1. A compound represented by the following general formula (1): 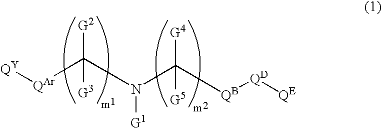
wherein
G¹ represents a hydrogen atom, or a C1-C4 alkyl group, with the proviso that the alkyl group may be substituted with one to three X^{G1}s, and when the alkyl group is substituted with two or more X^{G1}s, X^{G1}s may be the same or different;
X^{G1} represents a group selected from the group consisting of —OH, —CO₂H, —SO₃H, —PO₃H₂, and —OPO(OR^{G1})₂;
R^{G1} independently represents a hydrogen atom, or a C1-C4 alkyl group;
G² represents a hydrogen atom, a fluorine atom, a chlorine atom, or a C1-C4 alkyl group, with the proviso that the alkyl group may be substituted with one to three X^{G2}s, and when the alkyl group is substituted with two or more X^{G2}s, X^{G2}s may be the same or different;
X^{G2} represents a group selected from the group consisting of —OH, —CO₂H, —SO₃H, —PO₃H₂, and —OPO(OR^{G2})₂;
R^{G2} independently represents a hydrogen atom, or a C1-C4 alkyl group;
G³ represents a hydrogen atom, a fluorine atom, a chlorine atom, or a C1-C4 alkyl group, with the proviso that the alkyl group may be substituted with one to three X^{G3}s, and when the alkyl group is substituted with two or more X^{G3}s, X^{G3}s may be the same or different;
X^{G3} represents a group selected from the group consisting of —OH, —CO₂H, —SO₃H, —PO₃H₂, and —OPO(OR^{G3})₂;
R^{G3} independently represents a hydrogen atom, or a C1-C4 alkyl group;
G⁴ and G⁵, which may be the same or different, each independently represent a hydrogen atom, a fluorine atom, or a chlorine atom;
Q^{Ar} represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X^{QAr}s, and when these groups are substituted with two X^{QAr}s, X^{QAr}s may be the same or different;
X^{QAr} represents a fluorine atom, a chlorine atom, a bromine atom, a trifluoromethyl group, —OR^{XQAr}, —SR^{XQAr}, or —R^{XQAr};
R^{XQAr} represents a hydrogen atom, a C1-C6 alkyl group, a C3-C6 cycloalkyl group, or a phenyl group;
Q^{B} represents B^{Q1}, B^{Q2}, B³ or B⁴;
B^{Q1} represents a divalent group obtained by removing two hydrogen atoms from a compound selected from the group consisting of a 3- to 8-membered saturated ring compound composed of carbon atoms, a 3- to 8-membered partially unsaturated ring compound composed of carbon atoms, and a 3- to 8-membered unsaturated ring compound composed of carbon atoms, while these groups may be substituted to a possible extent with one to four X^{BQ1}s, and when these groups are substituted with two or more X^{BQ1}s, X^{BQ1}s may be the same or different;
X^{BQ1} represents a group selected from the group consisting of —OH, —CO₂H, —SO₃H, —PO₃H₂, —OPO(OR^{XBQ1})₂, a 1H-tetrazol-5-yl group, a fluorine atom, a chlorine atom, an amino group, -G^{XB1}, —OG^{XB1} and —NG_{XBG}G^{XB1′}, or represents a group selected from the group consisting of the following formulas (B3-1) to (B3-5): 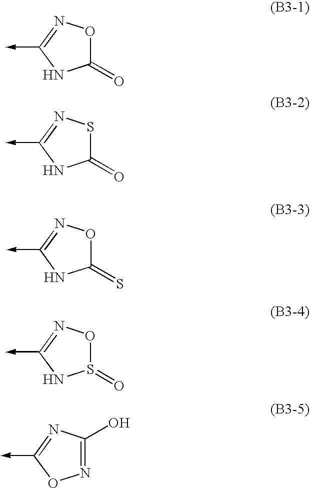
wherein an arrow represents a bonding position;
G^{XB1} and G^{XB1′}, which may be the same or different, each independently represent a C1-C4 alkyl group which may be substituted with 1 to 5 substituents selected from the group consisting of a halogen atom, a hydroxyl group, and an amino group;
R^{XBQ1} independently represents a hydrogen atom, or a C1-C4 alkyl group;
B^{Q2} represents a C1-C4 alkylene group, a C2-C4 alkenylene group, or a C2-C4 alkynylene group, while such group may be substituted to a possible extent with one to four X^{B2}s, and when these groups are substituted with two or more X^{B2}s, X^{B2}s may be the same or different;
X^{B2} represents a group selected from the group consisting of —OH, —CO₂H, —SO₃H, —PO₃H², —OPO(OR^{XBQ2})₂, a 1H tetrazol-5-yl group, a fluorine atom, a chlorine atom, an amino group, -G^{XB2}, —OG^{XB2} and NG^{XB2}G^{XB2};
G^{XB2} and G^{XB2′}, which may be the same or different, each independently represent a C1-C4 alkyl group which may be substituted with 1 to 5 substituents selected from a halogen atom, a hydroxyl group, and an amino group;
R^{XBQ2} independently represents a hydrogen atom, or a C1-C4 alkyl group;
B³ represents a divalent group obtained by removing two hydrogen atoms from a compound that has a number of ring constituting atoms of 3 to 8, and that is obtained by substituting one to two carbon atoms in the ring of a compound selected from the group consisting of a saturated monocyclic hydrocarbon ring compound, a partially saturated monocyclic hydrocarbon ring compound, and a monocyclic aromatic hydrocarbon ring compound by an oxygen atom, a sulfur atom or a nitrogen atom, while these groups may be substituted with one to four X^{B3}s, and when these groups are substituted with two or more X^{B3}s, X^{B3}s may be the same or different;
X^{B3} represents a group selected from the group consisting of —OH, —CO₂H, —SO₃H, —PO₃H₂, —OPO(OR^{XBQ3})₂, a 1H-tetrazol-5-yl group, a fluorine atom, a chlorine atom, an amino group, -G^{XB3}, OG^{XB3} and —NG^{XB3}G^{XB3′};
R^{XBQ3} independently represents a hydrogen atom, or a C1-C4 alkyl group;
G^{XB3} and G^{XB3′}, which may be the same or different, each independently represent a C1-C4 alkyl group which may be substituted with 1 to 5 substituents selected from a halogen atom, a hydroxyl group, and an amino group;
B⁴ represents a divalent group obtained by removing two hydrogen atoms from a compound that has a number of ring constituting atoms of 7 to 11, and that is obtained by substituting 1 to 5 carbon atoms in the ring of a compound selected from the group consisting of a saturated bicyclic hydrocarbon ring compound, a partially saturated bicyclic hydrocarbon ring compound, and a bicyclic aromatic hydrocarbon ring compound by an oxygen atom, a sulfur atom or a nitrogen atom, while these groups may be substituted to a possible extent with one to four X^{B4}s, and when these groups are substituted with two or more X^{B4}s, X^{B4}s may be the same or different;
X^{B4} represents a group selected from the group consisting of —OH, —CO₂H, —CH₂CO₂H, —SO₃H, —PO₃H₂, —PO₂H₂, —OPO(OR^{XBQ4})₂, a 1H-tetrazol-5-yl group, a fluorine atom, a chlorine atom, an amino group, -G^{XB4}, —OG^{XB4} and —NG^{XB4}G^{XB4′}.
R^{XBQ4} independently represents a hydrogen atom, or a C1-C4 alkyl group;
G^{XB4} and G^{XB4′}, which may be the same or different, each independently represent a C1-C4 alkyl group which may be substituted with 1 to 5 substituents selected from the group consisting of a halogen atom, a hydroxyl group, and an amino group;
Q^{D} represents a single bond, or a C1-C3 alkylene group which may be substituted with one to six fluorine atoms or chlorine atoms;
Q^{E} represents a group selected from the group consisting of —OH, —CO₂R^{QE}, —CH₂CO₂R^{QE}, —SO₃H, —PO₃H₂, —PO₂H₂, —OPO(OR^{QE})₂ and a 1H-tetrazol-5-yl group, or represents a group selected from the group consisting of the following formulas (B3-1) to (133-5): 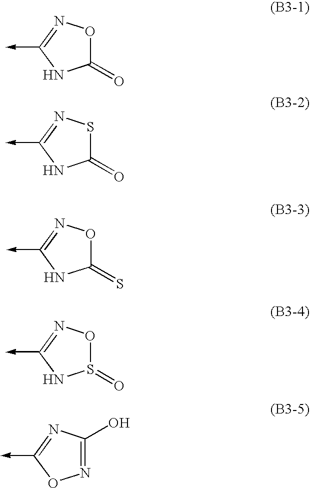
wherein an arrow represents a bonding position;
R^{QE} independently represents a hydrogen atom, a C1-C4 alkyl group, —(CH₂)_{mQ}N(R^{QE1})(R^{QE2}), or —C(R^{QE3})₂OC(O)A^{QE}R^{QE4};
m^{Q} denotes an integer of 2 or 3;
R^{QE1} and R^{QE2}, which may be the same or different, each independently represent a methyl group, an ethyl group, or a propyl group, or R^{QE1} and R^{QE2} are joined to form a 3- to 6-membered ring together with a nitrogen atom, thus representing a saturated nitrogen-containing cycloalkyl group, or to form a morpholino group together with the nitrogen atom;
R^{QE3} independently represents a hydrogen atom, a methyl group, an ethyl group, or a propyl group;
R^{QE4} represents a C1-C4 alkyl group, a C3-C6 cycloalkyl group, or a phenyl group;
A^{QE} represents a single bond or an oxygen atom;
Q^{Y} represents Q^{W}-Q^{T}-Q^{Z}-(CG⁶G⁷)_{nQ}-Q^{V}-;
Q^{W} represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{QW}R^{QW2}, a C3-C7 cycloalkoxy group which may be substituted with NR^{QW}R^{QW2}, a C1-C6 alkoxy group which may be substituted with a C3-C7 cycloalkyl group in which one to two carbon atoms in the ring may be substituted with a nitrogen atom or atoms, or a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X^{QW}s;
X^{QW} may be the same or different, when the monovalent group is substituted with two X^{QW}s;
X^{QW} represents a fluorine atom, a chlorine atom, a trifluoromethyl group, a cyano group, —OR^{XQW}, —SR^{XQW}, or —R^{XQW};
R^{XQW} represents a hydrogen atom, a C1-C6 alkyl group, a C3-C6 cycloalkyl group, or a phenyl group;
R^{QW} and R^{QW2}, which may be the same or different, each independently represent a hydrogen atom, or a C1-C4 alkyl group;
Q^{T} represents a single bond, a C1-C6 alkylene group, —O—, —OCH₂—, —S—, —SO—, —SO₂—, —NR^{QT}—, —NR^{QT}NHCO—, or —CONR^{QT}—;
R^{QT} represents a hydrogen atom, or a C1-C4 alkyl group;
QZ is a single bond, or represents a C1-C6 alkylene group or a C3-C6 cycloalkylene group, or a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the Q^{Z} may be further substituted with one to four X^{QZ}s, and when Q^{Z} is substituted with two or more X^{QZ}s, X^{QZ}s may be the same or different;
X^{QZ} represents a fluorine atom, a chlorine atom, a trifluoromethyl group, a cyano group, —OR^{XQZ}, —SR^{XQZ}, or R^{XQZ};
R^{XQZ} represents a hydrogen atom, a C1-C6 alkyl group, a C3-C6 cycloalkyl group, or a phenyl group, with the proviso that the C1-C6 alkyl group, the C3-C6 cycloalkyl group, and the phenyl group may be respectively substituted with fluorine atoms;
G⁶ and G⁷, which may be the same or different, each independently represent a hydrogen atom, or a C1-C4 alkyl group which may be substituted with 1 to 5 halogen atoms;
Q^{V} represents a single bond, —CO—, —COCR^{QV}-, —CR^{QV}R^{QV2}—, —S—, —SO—, —SO₂—, —SCH₂—, —SOCH₂—, —SO₂CH₂—, —O—, —CR^{QV}OR^{QV2}—, —CR^{QV}(OR^{QV2})CR^{QV3}R^{QV4}—, —CR^{QV}R^{QV2}CR^{QV3}(OR^{QV4})—, —C≡C—, —CR^{QV}═CR^{QV2}—, —NR^{QV}—, —NR^{QV}NHCO—, —CONR^{QV}—, or phenylene, or a divalent group obtained by removing two hydrogen atoms from oxadiazole or thiadiazole;
R^{QV}, R^{QV2}, R^{QV3}, and R^{QV4}, which may be the same or different, each independently represent a hydrogen atom, or a C1-C4 alkyl group;
n^{Q} denotes an integer of 0 to 2, with the proviso that when n^{Q} denotes 0, n^{Q} means a single bond;
m¹ denotes an integer of 1 to 3; and
m² denotes an integer of 0 to 3, with the proviso that when m² denotes 0, m² means a single bond,
or a possible stereoisomer or racemic body thereof, or a pharmacologically acceptable salt, hydrate or solvate of the compound, the stereoisomer or the racemic body, or a prodrug thereof.

2. A compound represented by the following formula (2): 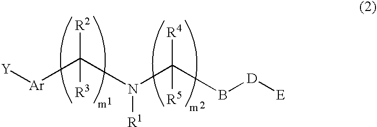
wherein
R¹, R², and R³, which may be the same or different, each independently represent a hydrogen atom, or a C1-C4 alkyl group;
R⁴ and R⁵, which may be the same or different, each independently represents a hydrogen atom, a fluorine atom, or a chlorine atom;
Ar represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, while these groups may be substituted with one to two X¹s, and when these groups are substituted with two X¹s, X¹s may be the same or different;
X¹ represents a fluorine atom, a chlorine atom, a bromine atom, a trifluoromethyl group, —OR^{X1}, —SR^{X1} or —R^{X1};
R^{X1} represents a hydrogen atom, a C1-C6 alkyl group, a C3-C6 cycloalkyl group, or a phenyl group;
B represents B¹ or B²;
B¹ represents a divalent group obtained by removing two hydrogen atoms from a compound selected from the group consisting of a 3- to 7-membered saturated ring compound composed of carbon atoms, a 3- to 7-membered partially unsaturated ring compound composed of carbon atoms, and a 3- to 7-membered unsaturated ring compound composed of carbon atoms, while these groups may be substituted to a possible extent with one to two X²s, and when these groups are substituted with two X²s, X²s may be the same or different;
X² represents a hydroxyl group, or a carboxyl group;
B represents a C1-C4 alkylene group, a C2-C4 alkenylene group, or a C2-C4 alkynylene group, while such group may be substituted with one to two X³s, and when the group is substituted with two X³s, X³s may be the same or different;
X³ represents a fluorine atom, a carboxyl group, or a C1-C4 alkyl group which may be substituted with a hydroxyl group or a carboxyl group;
D represents a single bond, a methylene group or an ethylene group;
E represents a hydroxyl group, —CO₂R^{E}, or a 1H-tetrazol-5-yl group;
R^{E} represents a hydrogen atom, a C1-C4 alkyl group, —(CH₂)ₘN(R^{E1})(R^{E2}), or —C(R^{E3})₂OC(O)A^{E}R^{E4};
m denotes an integer of 2 or 3;
R^{E1} and R^{E2}, which may be the same or different, each independently represents a methyl group, an ethyl group or a propyl group, or R^{E1} and R^{E2} are joined to form a 3- to 6-membered ring together with a nitrogen atom, thus representing a saturated nitrogen-containing cycloalkyl group, or to form a morpholino group together with the nitrogen atom;
R^{E3} represents a hydrogen atom, a methyl group, an ethyl group, or a propyl group;
R^{E4} represents a C1-C4 alkyl group, a C3-C6 cycloalkyl group, or a phenyl group;
A^{E} represents a single bond or an oxygen atom;
Y represents W-T-Z-(CR⁶R⁷)ₙ—V—;
W represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{W}R^{W2}, a C3-C7 cycloalkoxy group which may be substituted with NR^{W}R^{W2}, or a C1-C6 alkoxy group which may be substituted with a C3-C7 cycloalkyl group in which one to two carbon atoms in the ring may be substituted with a nitrogen atom or atoms, or a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, may be substituted with one to two X⁴s, and when the monovalent group is substituted with two X⁴s, X⁴s may be the same or different;
X⁴ represents a fluorine atom, a chlorine atom, a trifluoromethyl group, a trifluoromethoxy group, a cyano group, —OR^{X4}, —SR^{X4}, or —R^{X4};
R^{X4} represents a hydrogen atom, a C1-C6 alkyl group, a C3-C6 cycloalkyl group, or a phenyl group;
R^{W} and R^{W2}, which may be the same or different, each independently represent a hydrogen atom, or a C1-C6 alkyl group;
T represents a single bond, a C1-C6 alkylene group, —O—, —OCH₂—, —S—, —SO—, —SO₂—, —NR^{T}—, —NR^{T}NHCO—, or —CONR^{T}—;
R^{T} represents a hydrogen atom, or a C1-C6 alkyl group;
Z represents a C3-C6 cycloalkylene group, or represents a divalent group obtained by removing two hydrogen atoms from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that Z may be further substituted with one to four X⁵s, and when Z is substituted with two or more X⁵s, X⁵s may be the same or different;
X⁵ represents a fluorine atom, a chlorine atom, a trifluoromethyl group, a cyano group, —OR^{X5}, —SR^{X5} or —R^{X5};
R^{X5} represents a hydrogen atom, a C1-C6 alkyl group, a C3-C6 cycloalkyl group, or a phenyl group, with the proviso that the C1-C6 alkyl group, the C3-C6 cycloalkyl group and the phenyl group may be respectively substituted with a fluorine atom(s);
R⁶ and R⁷, which may be the same or different, each independently represent a hydrogen atom, or a C1-C4 alkyl group;
V represents a single bond, —CO—, —COCR^{V}—, —CR^{V}R^{V2}—, —S—, —SO—, —SO₂—, —SCH₂—, —SOCH₂—, —SO₂CH₂—, —O—, —CR^{V}OR^{V2}—, —CR^{V}(OR^{V2})CR^{V3}R^{V4}—, —CR^{V}R^{V2}CR^{V3}(OR^{V4})—, —C≡C—, —CR^{V}═CR^{V2}—, —NR^{V}—, —NR^{V}NHCO—, or —CONR^{V}—, or represents a divalent group obtained by removing two hydrogen atoms from oxadiazole or thiadiazole;
R^{V}, R^{V2}, R^{V3}, and R^{V4}, which may be the same or different, each independently represent a hydrogen atom, or a C1-C4 alkyl group;
n denotes an integer of 0 to 2, with the proviso that when n denotes 0, n means a single bond;
ml denotes an integer of 1 to 3; and
m² denotes an integer of 0 to 3, with the proviso that when m² denotes 0, m² means a single bond,
or a possible stereoisomer or racemic body thereof, or a pharmacologically acceptable salt, hydrate or solvate of the compound, the stereoisomer or the racemic body, or a prodrug thereof.

3. A compound represented by the following general formula (3): 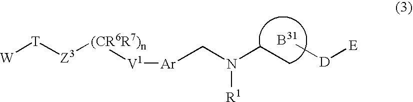
wherein W, T, R⁶, R⁷, n, Ar, R¹, D, and E have the same meaning as the defined above; B³¹ represents a divalent group obtained by removing two hydrogen atoms from a 3- to 7-membered saturated ring compound composed of carbon atoms; Z³ represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine, and imidazo[1,2-a]pyridine;
with the proviso that Z³ may be further substituted with one to four X^{Z3}s, and when Z³ is substituted with two or more X^{Z3}s, X^{Z3}s may be the same or different;
X^{Z3} represents a fluorine atom, a chlorine atom, a trifluoromethyl group, a cyano group, —OR^{XZ3}, —SR^{XZ3}, or —R^{XZ3};
R^{XZ3} represents a hydrogen atom, a C1-C6 alkyl group, a C3-C6 cycloalkyl group, or a phenyl group, with the proviso that the C1-C6 alkyl group, the C3-C6 cycloalkyl group, the phenyl group may be respectively substituted with a fluorine atom(s);
V¹ represents a single bond, —CO—, —COCR^{V1}R^{V12}—, —CR^{V1}R^{V12}—O—, —CR^{V1}OR^{V12}—, —CR^{V1}(OR^{V12})CR^{V13}R^{V14}, —CR^{V1}R^{V12}CR^{V13}(OR^{V14})—, C≡C—, —CR^{V1}═CR^{V12}, —NR^{V1}—, —NR^{V1}NHCO—, or —CONR^{V1}—, or represents a divalent group obtained by removing two hydrogen atoms from oxadiazole or thiadiazole; and
R^{V1}, R^{V12}, R^{V13}, and R^{V14}, which may be the same or different, each independently represent a hydrogen atom, or a C1-C4 alkyl group,
or a possible stercoisomer or racemic body thereof, or a pharmacologically acceptable salt, hydrate or solvate of the compound, the stereoisomer or the racemic body, or a prodrug thereof.

4. The compound according to claim 3, wherein B³¹ represents a divalent group obtained by removing two hydrogen atoms from a 4-membered saturated ring compound composed of carbon atoms,
or a possible stereoisomer or racemic body thereof, or a pharmacologically acceptable salt, hydrate or solvate of the compound, the stereoisomer or the racemic body, or a prodrug thereof.

5. The compound according to claim 3 or claim 4, wherein D represents a single bond, E represents CO₂R^{E} (provided that R^{E} has the same meaning as the defined above),
or a possible stereoisomer or racemic body thereof, or a pharmacologically acceptable salt, hydrate or solvate of the compound, the stereoisomer or the racemic body, or a prodrug thereof.

6. The compound according to claim 3,
wherein Ar represents a divalent group obtained by removing two hydrogen atoms from a compound selected from the group consisting of benzene and thiophene,
or a possible stereoisomer or racemic body thereof, or a pharmacologically acceptable salt, hydrate or solvate of the compound, the stereoisomer or the racemic body, or a prodrug thereof.

7. The compound according to claim 3, wherein V¹ represents a divalent group obtained by removing two hydrogen atoms from oxadiazole or thiadiazole,
or a possible stereoisomer or racemic body thereof, or a pharmacologically acceptable salt, hydrate or solvate of the compound, the stereoisomer or the racemic body, or a prodrug thereof.

8. The compound according to claim 3, wherein W represents a monovalent group obtained by removing one hydrogen atom from a compound selected from the group consisting of benzene, furan and thiophene, each of which may be substituted with one or two groups each independently selected from the group consisting of a fluorine atom, a chlorine atom, a cyano group and a trifluoromethyl group,
or a possible stereoisomer or racemic body thereof or a pharmacologically acceptable salt, hydrate or solvate of the compound, the stereoisomer or the racemic body, or a prodrug thereof.

9. The compound according to claim 3, wherein W represents a hydrogen atom, a C1-C6 alkyl group which may be substituted with 1 to 7 fluorine atoms, or a C3-C7 cycloalkyl group which may be substituted with 1 to 7 fluorine atoms,
or a possible stereoisomer or racemic body thereof, or a pharmacologically acceptable salt, hydrate or solvate of the compound, the stereoisomer or the racemic body, or a prodrug thereof.

10. The compound according to claim 3, wherein Z³ represents a divalent group obtained by removing two hydrogen atoms from a compound selected from benzene and thiophene, each of which may be substituted with one or two groups each independently selected from the group consisting of a fluorine atom, a chlorine atom, a cyano group, and a trifluoromethyl group,
or a possible stereoisomer or racemic body thereof, or a pharmacologically acceptable salt, hydrate or solvate of the compound, the stereoisomer or the racemic body, or a prodrug thereof.

11. The compound according to claim 3, wherein
R¹ represents a hydrogen atom;
Ar represents a divalent group obtained by removing two hydrogen atoms from benzene, while the group may be substituted with one to two X¹s, and when the group is substituted with two X¹s, X¹s may be the same or different, with X¹ representing a fluorine atom, a chlorine atom, a bromine atom, —OR^{X1}, or —R^{X1};
R^{X1} represents a hydrogen atom, a methyl group, an ethyl group, a propyl group, or an isopropyl group;
B³¹ represents a divalent group obtained by removing two hydrogen atoms from a 4- to 5-membered saturated ring compound composed of carbon atoms;
E represents CO₂R^{E}; R^{E} represents a hydrogen atom, a methyl group, an ethyl group, —(CH₂)ₘN(R^{E1})(R^{E2}), or —C(R^{E3})₂OC(O)A^{E}R^{E4}; m denotes an integer of 2 or 3; R^{E1} and R^{E2} may be the same or different, and each independently represent a methyl group, an ethyl group or a propyl group, or R^{E1} and R^{E2} are joined to form a 3- to 6-membered ring together with a nitrogen atom, thus representing a saturated nitrogen-containing cycloalkyl group, or to form a morpholino group together with the nitrogen atom; R^{E3} represents a hydrogen atom, a methyl group, an ethyl group, or a propyl group; R^{E4} represents a C1-C4 alkyl group, a C3-C6 cycloalkyl group, or a phenyl group; A^{E} represents a single bond or an oxygen atom;
W represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene and pyridine, with the proviso that the monovalent group obtained by removing one hydrogen atom from benzene, naphthalene, furan, thiophene and pyridine, may be substituted with one to two X⁴s, and when the group is substituted with two X⁴s, X⁴s may be the same or different, with X⁴ representing a fluorine atom, a trifluoromethyl group, a trifluoromethoxy group, a cyano group, —OR^{X4} or —R^{X4}, and R^{X4} representing a hydrogen atom, a methyl group, an ethyl group, a propyl group, an isopropyl group, a cyclopentyl group, a cyclohexyl group or a phenyl group;
T represents a single bond, a methylene group, an ethylene group, or —O—;
Z³ represents a C5-C6 cycloalkylene group, or a divalent group obtained by removing two hydrogen atoms from a compound selected from the group consisting of benzene, thiophene, and pyridine, while Z³ may be substituted with one to four X^{Z3}s, and when Z³ is substituted with two or more X^{Z3}s, X^{Z3} may be the same or different, with X^{Z3} representing a fluorine atom, a trifluoromethyl group, a cyano group, —OR^{XZ3} or —R^{XZ3}, and R^{XZ3} representing a methyl group, an ethyl group, or a propyl group;
V¹ represents a divalent group obtained by removing two hydrogen atoms from oxadiazole or thiadiazole; and
n denotes 0 or 1,
or a possible stereoisomer or racemic body thereof, or a pharmacologically acceptable salt, hydrate or solvate of the compound, the stereoisomer or the racemic body, or a prodrug thereof.

12. A compound represented by the following general formula (6): 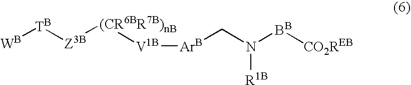
wherein
R^{1B} represents a hydrogen atom, or a C1-C4 alkyl group;
Ar^{B} represents a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine;
these groups may be substituted with one to two X^{1B}s, and when these groups are substituted with two X^{1B}s, X^{1B}s may be the same or different;
X^{1B} represents a fluorine atom, a chlorine atom, a bromine atom, a trifluoromethyl group, —OR^{X1B}, —SR^{X1B}, or R^{X1B};
R^{X1B} represents a hydrogen atom, a C1-C6 alkyl group, a C3-C6 cycloalkyl group, or a phenyl group;
B represents a C2 alkylene group, while the group may be substituted to a possible extent with one to four X^{BB}s, and when the group is substituted with two or more X^{BB}s, X⁸⁸s may be the same or different;
XBB represents a fluorine atom, or a C1-C4 alkyl group which may be substituted with one to five G^{XBB}s, and when the alkyl group is substituted with two or more G^{XBB}s, G^{XBB}s may be the same or different;
G^{XBB} represents a halogen atom, a hydroxyl group or an amino group;
R^{EB} represents a hydrogen atom, a C1-C4 alkyl group, —(CH₂)_{mB}N(R^{EB1})(R^{EB2}), or —C(R^{EB3})₂OC(O)A^{EB}R^{EB4};
m^{B} denotes an integer of 2 or 3;
R^{EB1} and REB², which may be the same or different, each independently represent a methyl group, an ethyl group or a propyl group, or R^{EB1} and REB² are joined to form a 3- to 6-membered ring together with a nitrogen atom, thus representing a saturated nitrogen-containing cycloalkyl group, or to form a morpholino group together with the nitrogen atom;
R^{EB3} represents a hydrogen atom, a methyl group, an ethyl group or a propyl group;
R^{EB4} represents a C1-C4 alkyl group, a C3-C6 cycloalkyl group or a phenyl group;
A^{EB} represents a single bond or an oxygen atom;
V^{1B} represents a single bond, or a divalent group obtained by removing two hydrogen atoms from a compound selected from the group consisting of oxadiazole and thiadiazole;
W^{B} represents a hydrogen atom, a C1-C6 alkyl group which may be substituted with 1 to 7 fluorine atoms, a C3-C7 cycloalkyl group which may be substituted with 1 to 7 fluorine atoms, a C1-C6 alkoxy group which may be substituted with NR^{WB}R^{WB2}, a C3-C7 cycloalkoxy group which may be substituted with NR^{WB}R^{WB2}, or a C1-C6 alkoxy group which may be substituted with a C3-C7 cycloalkyl group in which one to two carbon atoms in the ring may be substituted with a nitrogen atom or atoms, or represents a monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring, with the proviso that the monovalent group obtained by removing one hydrogen atom from a compound selected from the group consisting of a monocyclic aromatic hydrocarbon ring compound, a monocyclic aromatic heterocyclic compound, a bicyclic aromatic hydrocarbon ring compound, a bicyclic aromatic heterocyclic compound, a bicyclic compound obtained by fusing a saturated hydrocarbon ring with a monocyclic aromatic hydrocarbon ring, a bicyclic compound obtained by fusing a saturated heterocyclic ring with a monocyclic aromatic hydrocarbon ring, a bicyclic compound obtained by fusing a saturated hydrocarbon ring with a monocyclic aromatic heterocyclic ring, and a bicyclic compound obtained by fusing a saturated heterocyclic ring with a monocyclic aromatic heterocyclic ring may be substituted with one to two X^{4B}s, and when the group is substituted with two X^{4B}s, X^{B}s may be the same or different;
X^{4B} represents a fluorine atom, a chlorine atom, a trifluoromethyl group, a trifluoromethoxy group, a cyano group, —OR^{X4B}, —SR^{X4B}, or —R^{X4B},
R^{X4B} represents a hydrogen atom, a C1-C6 alkyl group, a C3-C6 cycloalkyl group, or a phenyl group;
R^{WB} and R^{WB2}, which may be the same or different, each independently represent a hydrogen atom, or a C1-C6 alkyl group;
T^{B} represents a single bond, a C1-C6 alkylene group, —O—, —OCH₂—, —NR^{TB}—, —NR^{TB}NHCO—, or —CONR^{TB}—;
R^{TB} represents a hydrogen atom, or a C1-C6 alkyl group;
Z^{3B} represents a C3-C6 cycloalkylene group, or a divalent group obtained by removing two hydrogen atoms from a compound selected from the group consisting of benzene, furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine, thiazine, pentalene, azulene, naphthalene, benzofuran, benzo[b]thiophene, indole, isoindole, indolizine, 1H-indazole, 2H-indazole, 1H-benzimidazole, benzoxazole, benzo[d]isoxazole, benzo[c]isoxazole, benzothiazole, benzo[d]isothiazole, benzo[c]isothiazole, 1H-benzotriazole, benzo[1,2,5]thiadiazole, quinoline, isoquinoline, cinnoline, quinazoline, quinoxaline, phthalazine and imidazo[1,2-a]pyridine;
with the proviso that Z^{3B} may be further substituted with one to four X^{Z3B}s, and when Z^{3B} is substituted with two or more X^{Z3B}s, X^{Z3B}s may be the same or different;
X^{Z3B} represents a fluorine atom, a chlorine atom, a trifluoromethyl group, a cyano group, —OR^{XZ3B}, —SR^{XZ3B}, or —R^{XZ3B};
R^{XZ3B} represents a hydrogen atom, a C1-C6 alkyl group, a C3-C6 cycloalkyl group or a phenyl group, provided that the C1-C6 alkyl group, the C3-C6 cycloalkyl group and the phenyl group may be respectively substituted with a fluorine atom(s);
when V^{1B} is a single bond, n^{B} denotes 0, and Z^{3B} is a single bond, or a divalent group obtained by removing two hydrogen atoms from a compound selected from the group consisting of furan, thiophene, pyrrole, oxazole, isoxazole, oxadiazole, thiazole, isothiazole, thiadiazole, imidazole, pyrazole, pyran, pyridine, pyridazine, pyrimidine, pyrazine, oxazine and thiazine;
when V^{1B} is a single bond, and Z^{3B} is a single bond, T^{B} represents a single bond, and W^{B} represents a divalent group obtained by removing one hydrogen atom from a monocyclic aromatic heterocyclic compound;
R^{6B} and R^{7B}, which may be the same or different, each independently represent a hydrogen atom or a C1-C4 alkyl group; and
n^{B} denotes an integer of 0 to 2, with the proviso that when n^{B} denotes 0, it means a single bond,
or a possible stereoisomer or racemic body thereof, or a pharmacologically acceptable salt, hydrate or solvate of the compound, the stereoisomer or the racemic body, or a prodrug thereof.

13. The compound according to claim 12, wherein B^{B} represents a C2 alkylene group, while the group may be substituted with one to four C1-C4 alkyl groups, and when the group is substituted with two or more C1-C4 alkyl groups, the C1-C4 alkyl groups may be the same or different,
or a possible stereoisomer or racemic body thereof, or a pharmacologically acceptable salt, hydrate or solvate of the compound, the stereoisomer or the racemic body, or a prodrug thereof.

14. The compound according to claim 12 or claim 13, wherein Ar^{B} represents a divalent group obtained by removing two hydrogen atoms from a compound selected from the group consisting of benzene and thiophene,
or a possible stereoisomer or racemic body thereof, or a pharmacologically acceptable salt, hydrate or solvate of the compound, the stereoisomer or the racemic body, or a prodrug thereof.

15. The compound according to claim 12, wherein V^{1B} represents a divalent group obtained by removing two hydrogen atoms from a compound selected from the group consisting of [1,2,4]-oxadiazole, [1,3,4]-oxadiazole, [1,2,4]-thiadiazole and [1,3,4]-thiadiazole,
or a possible stereoisomer or racemic body thereof or a pharmacologically acceptable salt, hydrate or solvate of the compound, the stereoisomer or the racemic body, or a prodrug thereof.

16. The compound according to claim 12, wherein V^{1B} represents a single bond; n^{B} denotes 0; and Z^{3B} represents a divalent group obtained by removing two hydrogen atoms from a compound selected from the group consisting of furan, pyrrole, oxazole, thiazole, isothiazole, pyran, pyridine, pyridazine, pyrimidine, pyrazine, oxazine and thiazine,
or a possible stereoisomer or racemic body thereof, or a pharmacologically acceptable salt, hydrate or solvate of the compound, the stereoisomer or the racemic body, or a prodrug thereof.

17. The compound according to claim 12, wherein B^{B} represents a C2 alkylene group; and Ar^{B} represents a divalent group obtained by removing two hydrogen atoms from benzene,
or a possible stereoisomer or racemic body thereof, or a pharmacologically acceptable salt, hydrate or solvate of the compound, the stereoisomer or the racemic body, or a prodrug thereof.

18. The compound according to claim 12, wherein Z^{3B} represents a C3-C6 cycloalkylene group, or a divalent group obtained by removing two hydrogen atoms from a compound selected from the group consisting of benzene, thiophene, oxadiazole, thiazole, isothiazole, thiadiazole, pyran, pyridine, pyridazine, pyrimidine and pyrazine,
or a possible stereoisomer or racemic body thereof, or a pharmacologically acceptable salt, hydrate or solvate of the compound, the stereoisomer or the racemic body, or a prodrug thereof.

19. The compound according to claim 12, wherein Z^{3B} represents a C3-C6 cycloalkylene group, or a divalent group obtained by removing two hydrogen atoms from a compound selected from the group consisting of benzene, thiophene and pyridine,
or a possible stercoisomer or racemic body thereof, or a pharmacologically acceptable salt, hydrate or solvate of the compound, the stercoisomer or the racemic body, or a prodrug thereof.

20. The compound according to claim 12, wherein
Ar^{B} represents a divalent group obtained by removing two hydrogen atoms from benzene, while the group may be substituted with one to two X^{1B}s, and when the group is substituted with two X^{1B}s, X^{1B}s may be the same or different, with X^{1B} representing a fluorine atom, a chlorine atom, a bromine atom, —OR^{X1B} or —R^{X1B}, and R^{X1B} representing a hydrogen atom, a methyl group, an ethyl group, a propyl group or an isopropyl group;
B^{B} represents a C2 alkylene group;
R^{EB} represents a hydrogen atom, a methyl group, an ethyl group, —(CH₂)_{mB}N(R^{EB1})(R^{EB2}) or —C(R^{EB3})₂OC(O)A^{EB}R^{EB4};
W^{B} represents a hydrogen atom, a C1-C6 alkyl group which may be substituted with 1 to 7 fluorine atoms, a C3-C7 cycloalkyl group which may be substituted with 1 to 7 fluorine atoms, or a monovalent group obtained by removing one hydrogen atom from a compound selected from the group consisting of benzene, naphthalene, furan, thiophene, and pyridine, with the proviso that the monovalent group obtained by removing one hydrogen atom from a compound selected from the group consisting of benzene, naphthalene, furan, thiophene, and pyridine may be substituted with one to two X^{4B}s, and when the group is substituted with two X^{4B}s, X^{4B}s may be the same or different, with X^{4B} representing a fluorine atom, a trifluoromethyl group, a trifluoromethoxy group, a cyano group, OR^{X4B} or —R^{X4B}, and R^{X4B} representing a hydrogen atom, a methyl group, an ethyl group, a propyl group, an isopropyl group, a cyclopentyl group, a cyclohexyl group or a phenyl group;
T^{B} represents a single bond, a methylene group, an ethylene group or —O—;
Z^{3B} represents a divalent group obtained by removing two hydrogen atoms from a compound selected from the group consisting of benzene and pyridine,
with the proviso that Z^{3B} may be further substituted with one to four X^{Z3B}s, and when Z^{3B} is substituted with two or more X^{Z3B}s, X^{Z3B}s may be the same or different;
X^{Z3B} represents a fluorine atom, a chlorine atom, a trifluoromethyl group, a cyano group, OR^{XZ3B}, —SR^{XZ3B} or R^{XZ3B};
R^{XZ3B} represents a hydrogen atom, a C1-C6 alkyl group or a C3-C6 cycloalkyl group, provided that the C1-C6 alkyl group and the C3-C6 cycloalkyl group may be respectively substituted with a fluorine atom(s); and
V^{1B} represents a divalent group obtained by removing two hydrogen atoms from a compound selected from the group consisting of [1,2,4]-oxadiazole, [1,3,4]-oxadiazole, [1,2,4]-thiadiazole and [1,3,4]-thiadiazole,
or a possible stereoisomer or racemic body thereof, or a pharmacologically acceptable salt, hydrate or solvate of the compound, the stereoisomer or the racemic body, or a prodrug thereof.

21. A pharmaceutical product comprising, as an active ingredient, the compound according to claim 2, 3, 12, or 20, or a possible stereoisomer or racemic body thereof, or a pharmacologically acceptable salt, hydrate or solvate of the compound, the stereoisomer or the racemic body, or a prodrug thereof.

22. An S1P1/Edg1 receptor activating agent comprising, as an active ingredient, the compound according claim 2, or a pharmacologically acceptable salt thereof.

23. A pharmaceutical product according to claim 2, which is a prophylactic and/or therapeutic agent for autoimmune diseases in a mammal.

24. A method for preventing and/or treating autoimmune diseases in a mammal, the method comprising administering to the mammal including human an effective amount of the compound according to claim 2, 3, 12, or 20, or a possible stereoisomer or racemic body thereof, or a pharmacologically acceptable salt, hydrate or solvate of the compound, the stereoisomer or the racemic body, or a prodrug thereof.
